(19) United States
(12) Patent Application Publication
(10) Pub. No.: US 2025/0215014 A1
(43) Pub. Date: Jul. 3, 2025

(54) KINASE INHIBITORS, PREPARATION METHODS AND USES THEREOF

(71) Applicants: InventisBio Co., Ltd., Shanghai (CN); InventisBio LLC, Short Hills, NJ (US)

(72) Inventors: Younong YU, Short Hills, NJ (US); Xianhai HUANG, Warren, NJ (US); Yong LUO, Shanghai (CN); Haotao NIU, Shanghai (CN); Hong YANG, Shanghai (CN); Jifang WENG, Shanghai (CN); Yaolin WANG, Shanghai (CN); Xing DAI, Shanghai (CN)

(21) Appl. No.: 18/852,245

(22) PCT Filed: Mar. 31, 2022
(86) PCT No.: PCT/CN2022/084377

§ 371 Date: Sep. 27, 2024

(51) Int. Cl. C07D 498/22; A61K 31/4375; A61K 31/438; A61K 31/4545; A61K 31/517; A61K 31/519; A61K 31/5383; C07D 413/14; C07D 471/04; C07D 471/14; C07D 498/16; C07D 519/00

(52) U.S. Cl. CPC C07D 498/22; A61K 31/4375; A61K 31/438; A61K 31/4545; A61K 31/517; A61K 31/519; A61K 31/5383; C07D 413/14; C07D 471/04; C07D 471/14; C07D 498/16; C07D 519/00

(57) ABSTRACT

Provided herein are novel compounds, for example, compounds having a Formula I, Formula II-1 to II-7, Formula III-1 to III-9, IV, or V, or a pharmaceutically acceptable salt thereof. Also provided herein are methods of preparing the compounds and methods of using the compounds, for example, in inhibiting DGKa and/or DGKz in a cell, and/or in treating various diseases such as cancer or viral infections.

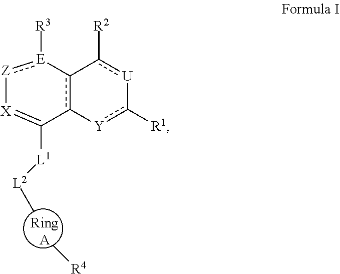

### BACKGROUND

### Field of the Disclosure

In various embodiments, the present disclosure generally relates to novel compounds, compositions comprising the same, methods of preparing and methods of using the same, e.g., for inhibiting DGKs and/or for treating a number of diseases or disorders, such as cancers or infections.

### Background

Diacylglycerol kinases (DGKs) have been indicated as serving as intracellular checkpoints. Inhibition of DGKs are expected to enhance T cell signaling pathways and T cell activation. See e.g., Riese M. J. et al., Journal of Biological Chemistry, (2011) 7: 5254-5265; Zha Y et al., Nature Immunology, (2006) 12:1343; Olenchock B. A. et al., (2006) 11: 1174-81). DGKa (DGK alpha) and DGKz (DGK zeta) have been viewed as targets for cancer immunotherapy. There remains a need for compounds useful as inhibitors of one or both of DGKa and DGKz.

### BRIEF SUMMARY

The present disclosure is based in part on Applicant's discovery of compounds that have activity as inhibitors of one or both of DGKa and DGKz. In various embodiments, the present disclosure provides novel compounds, pharmaceutical compositions, methods of preparing and using the same. Typically, the compounds herein are DGK inhibitors, such as DGKa and/or DGKz inhibitors. The compounds and compositions herein are useful for treating various diseases or disorders, such as cancer or viral infections.

In some embodiments, the present disclosure provides a compound of Formula I, II-1, I-2, II-3, II-4, II-5, II-6, II-7, III-1, III-2, III-3, III-4, III-5, III-6, III-7, III-8, III-9, IV, or V, or a pharmaceutically acceptable salt thereof, as defined herein. In some embodiments, the compound of Formula I can also be characterized as having a structure according to a subformula of Formula I-1, I-2, I-3, I-4, I-5, I-6, I-A, I-B, I-C, I-A-1, I-B-1, I-C-1, I-D, I-D-1, I-D-2, I-E, I-L-1, I-L-2, I-L-3, I-L-4, I-L-5, I-L-6, I-L-7, I-L-8, I-L-9, I-L-10, or I-L-11. In some embodiments, the compound of Formula II-3 can also be characterized as having a structure according to a subformula of Formula II-3-A, II-3-B, or II-3-C. In some embodiments, the compound of Formula III-2 can also be characterized as having a structure according to a subformula of Formula III-2-A, III-2-B, III-2-C, III-2-C-1, III-2-C-2, III-2-C-3, III-2-C-4, III-2-D, III-2-D-1, III-2-D-2, III-2-D-3, III-2-D-4, III-2-E-1, III-2-E-2, III-2-E-3, III-2-E-1a, III-2-E-2a, III-2-E-3a, III-2-E-1b, III-2-E-2b, or III-2-E-3b. In some embodiments, the compound of Formula III-3 can also be characterized as having a structure according to a subformula of Formula III-A. In some embodiments, the compound of Formula III-4 can also be characterized as having a structure according to a subformula of Formula III-4-A, III-4-B, III-4-C, III-4-D, III-4-C-1, III-4-C-2, III-4-C-3, III-4-C-4, III-4-D-1, or III-4-D-2. In some embodiments, the compound of Formula III-8 can also be characterized as having a structure according to a subformula of Formula III-8-A. In some embodiments, the compound of Formula V can also be characterized as having a structure according to a subformula of Formula V-1, V-2, or V-3. In some embodiments, the present disclosure also provides a compound selected from the compounds shown in Table A herein, or a pharmaceutically acceptable salt thereof. In some embodiments, the present disclosure also provides a compound selected from Compound Nos. 1-134 herein, or a pharmaceutically acceptable salt thereof.

Certain embodiments of the present disclosure are directed to a pharmaceutical composition comprising one or more of the compounds of the present disclosure (e.g., a compound of Formula I (e.g., Formula I-1, I-2, I-3, I-4, I-5, I-6, I-A, I-B, I-C, I-A-1, I-B-1, I-C-1, I-D, I-D-1, I-D-2, I-E, I-L-1, I-L-2, I-L-3, I-L-4, I-L-5, I-L-6, I-L-7, I-L-8, I-L-9, I-L-10, or I-L-11), II-1, I-2, II-3 (e.g., II-3-A, II-3-B, or II-3-C), II-4, II-5, II-6, II-7, III-1, III-2 (e.g., III-2-A, III-2-B, III-2-C, III-2-C-1, III-2-C-2, III-2-C-3, III-2-C-4, III-2-D, III-2-D-1, III-2-D-2, III-2-D-3, III-2-D-4, III-2-E-1, III-2-E-2, III-2-E-3, III-2-E-1a, III-2-E-2a, III-2-E-3a, III-2-E-1b, III-2-E-2b, or III-2-E-3b), III-3 (e.g., III-3-A), III-4 (e.g., III-4-A, III-4-B, III-4-C, III-4-D, III-4-C-1, III-4-C-2, III-4-C-3, III-4-C-4, III-4-D-1, or III-4-D-2), III-5, III-6, III-7, III-8 (e.g., III-8-A), III-9, IV, or V (e.g., V-1, V-2, or V-3), any of Compound Nos. 1-134, any compound selected from the compounds shown in Table A herein, or a pharmaceutically acceptable salt thereof) and optionally a pharmaceutically acceptable excipient. The pharmaceutical composition described herein can be formulated for various routes of administration, such as oral administration, parenteral administration, or inhalation etc.

Certain embodiments are directed to a method of treating a disease or disorder associated with the activity of DGKa, DGKz, or both DGKa and DGKz. In some embodiments, the method comprises administering to a subject in need thereof a therapeutically effective amount of a compound of the present disclosure (e.g., a compound of Formula I (e.g., Formula I-1, I-2, I-3, I-4, I-5, I-6, I-A, I-B, I-C, I-A-1, I-B-1, I-C-1, I-D, I-D-1, I-D-2, I-E, I-L-1, I-L-2, I-L-3, I-L-4, I-L-5, I-L-6, I-L-7, I-L-8, I-L-9, I-L-10, or I-L-11), II-1, I-2, II-3 (e.g., II-3-A, II-3-B, or II-3-C), II-4, II-5, II-6, II-7, III-1, III-2 (e.g., III-2-A, III-2-B, III-2-C, III-2-C-1, III-2-C-2, III-2-C-3, III-2-C-4, III-2-D, III-2-D-1, III-2-D-2, III-2-D-3, III-2-D-4, III-2-E-1, III-2-E-2, III-2-E-3, III-2-E-1a, III-2-E-2a, III-2-E-3a, III-2-E-1b, III-2-E-2b, or III-2-E-3b), III-3 (e.g., III-3-A), III-4 (e.g., III-4-A, III-4-B, III-4-C, III-4-D, III-4-C-1, III-4-C-2, III-4-C-3, III-4-C-4, III-4-D-1, or III-4-D-2), III-5, III-6, III-7, III-8 (e.g., III-8-A), III-9, IV, or V (e.g., V-1, V-2, or V-3), any of Compound Nos. 1-134, any compound selected from the compounds shown in Table A herein, or a pharmaceutically acceptable salt thereof) or a therapeutically effective amount of a pharmaceutical composition described herein. Diseases or disorders associated with the activity of DGKa, DGKz, or both DGKa and DGKz suitable to be treated with the method include any of those described herein.

In some embodiments, a method of treating cancer is provided. In some embodiments, the method comprises administering to a subject in need thereof a therapeutically effective amount of a compound of the present disclosure (e.g., a compound of Formula I (e.g., Formula I-1, I-2, I-3, I-4, I-5, I-6, I-A, I-B, I-C, I-A-1, I-B-1, I-C-1, I-D, I-D-1, I-D-2, I-E, I-L-1, I-L-2, I-L-3, I-L-4, I-L-5, I-L-6, I-L-7, I-L-8, I-L-9, I-L-10, or I-L-11), II-1, I-2, II-3 (e.g., II-3-A, II-3-B, or II-3-C), II-4, II-5, II-6, II-7, III-1, III-2 (e.g., III-2-A, III-2-B, III-2-C, III-2-C-1, III-2-C-2, III-2-C-3, III-2-C-4, III-2-D, III-2-D-1, III-2-D-2, III-2-D-3, III-2-D-4, III-2-E-1, III-2-E-2, III-2-E-3, III-2-E-1a, III-2-E-2a, III-2-E-3a, III-2-E-1b, III-2-E-2b, or III-2-E-3b), III-3 (e.g., III-3-A), III-4 (e.g., III-4-A, III-4-B, III-4-C, III-4-D, III-4-C-1, III-4-C-2, III-4-C-3, III-4-C-4, III-4-D-1, or III-4-D-2), III-5, III-6, III-7, III-8 (e.g., III-8-A), III-9, IV, or V (e.g., V-1, V-2, or V-3), any of Compound Nos. 1-134, any compound selected from the compounds shown in Table A herein, or a pharmaceutically acceptable salt thereof) or a therapeutically effective amount of a pharmaceutical composition described herein. In various embodiments, the cancer can be cancer of the colon, pancreatic cancer, breast cancer, prostate cancer, lung cancer, ovarian cancer, cervical cancer, renal cancer, cancer of the head and neck, lymphoma, leukemia, and/or melanoma.

In some embodiments, a method of treating viral infection is provided. In some embodiments, the method comprises administering to a subject in need thereof a therapeutically effective amount of a compound of the present disclosure (e.g., a compound of Formula I (e.g., Formula I-1, I-2, I-3, I-4, I-5, I-6, I-A, I-B, I-C, I-A-1, I-B-1, I-C-1, I-D, I-D-1, I-D-2, I-E, I-L-1, I-L-2, I-L-3, I-L-4, I-L-5, I-L-6, I-L-7, I-L-8, I-L-9, I-L-10, or I-L-11), II-1, I-2, II-3 (e.g., II-3-A, II-3-B, or II-3-C), II-4, II-5, II-6, II-7, III-1, III-2 (e.g., III-2-A, III-2-B, III-2-C, III-2-C-1, III-2-C-2, III-2-C-3, III-2-C-4, III-2-D, III-2-D-1, III-2-D-2, III-2-D-3, III-2-D-4, III-2-E-1, III-2-E-2, III-2-E-3, III-2-E-1a, III-2-E-2a, III-2-E-3a, III-2-E-1b, III-2-E-2b, or III-2-E-3b), III-3 (e.g., III-3-A), III-4 (e.g., III-4-A, III-4-B, III-4-C, III-4-D, III-4-C-1, III-4-C-2, III-4-C-3, III-4-C-4, III-4-D-1, or III-4-D-2), III-5, III-6, III-7, III-8 (e.g., III-8-A), III-9, IV, or V (e.g., V-1, V-2, or V-3), any of Compound Nos. 1-134, any compound selected from the compounds shown in Table A herein, or a pharmaceutically acceptable salt thereof) or a therapeutically effective amount of a pharmaceutical composition described herein.

The administering in the methods herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally.

The compounds of the present disclosure can be used as a monotherapy or in a combination therapy. In some embodiments, the combination therapy includes treating the subject with a targeted therapeutic agent, chemotherapeutic agent, therapeutic antibody, radiation, cell therapy, and/or immunotherapy. In some embodiments, the combination therapy includes treating the subject with an immune-oncology agent herein. In some embodiments, the combination therapy includes treating the subject with one or more additional antiviral agents.

It is to be understood that both the foregoing summary and the following detailed description are exemplary and explanatory only, and are not restrictive of the invention herein.

### DETAILED DESCRIPTION

In a broad aspect, the present disclosure provides compounds and compositions that are useful for inhibiting DGKs (diacylglycerol kinases), such as DGKa and/or DGKz, and/or treating or preventing various diseases or disorders described herein, e.g., cancer or infectious diseases such as viral infections.

### Compounds

Some embodiments of the present disclosure are directed to novel compounds. The compounds herein typically can be a DGK inhibitor, and useful for treating various diseases or disorders, such as those described herein, e.g., cancer or viral infections.

### Formula I

In some embodiments, the present disclosure provides a compound of Formula I, or a pharmaceutically acceptable salt thereof:

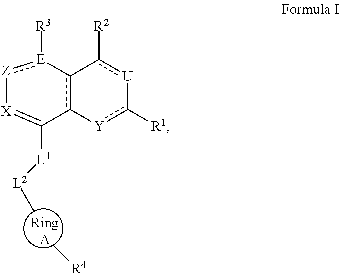

wherein:
   R¹ is hydrogen, halogen (e.g., F, Cl, or Br), CN, OH, COOH, CONH₂, NH₂, R^{A}, OR^{A}, COOR^{A}, NH(R^{A}), N(R^{A})₂, CONH(R^{A}), CON(R^{A})₂, SR^{A}, SOR^{A}, SO₂R^{A}, or P(O)(R^{A})₂, wherein R^{A} at each occurrence is independently optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
   R² is hydrogen, halogen (e.g., F, Cl, or Br), CN, OH, NH₂, R^{B}, OR^{B}, NH(R^{B}), or N(R^{B})₂, wherein R^{B} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, or optionally substituted 4-7 membered heterocyclyl;
   R³ is hydrogen, optionally substituted C₁₋₄ alkyl, optionally substituted C₃₋₆ cycloalkyl, or optionally substituted 4-7 membered heterocyclyl (such as a saturated 4-7 membered heterocyclyl);
   E is N;
   X is N or CR⁵, wherein R⁵ is hydrogen, halogen, CN, CONH₂, COOH, CONH(R^{C}), CON(R^{C})₂, OR^{C}, NO₂, or COOR^{C}, wherein R^{C} at each occurrence is independently an optionally substituted C₁₋₄ alkyl;
   U is N or CR⁶, wherein R⁶ is hydrogen, halogen (e.g., F, Cl, or Br), CN, OH, NH₂, R^{D}, OR^{D}, NH(R^{D}), N(R^{D})₂, COOH, CONH₂, COOR^{D}, CONH(R^{D}), CON(R^{D})₂, SR^{D}, SOR^{D}, SO₂R^{D}, or P(O)(R^{D})₂, wherein R^{D} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
   Y is N or CR⁷, wherein R⁷ is hydrogen, F, Cl, CN, optionally substituted C₁₋₄ alkyl, or optionally substituted C₁₋₄ heteroalkyl;
   Z is C(O), C(═N—OH), C(═N—O—(C₁₋₄ alkyl)), C(═N—NH₂), C(═N—NH—(C₁₋₄ alkyl)), C(═N—N(C₁₋₄ alkyl)(C₁₋₄ alkyl)), SO, SO₂, S(O)(═NH), or S(O)(═N—(C₁₋₄ alkyl);
   or Y and R¹ together are joined to form an optionally substituted ring selected from a 5 or 6 membered heteroaryl having 1-3 ring heteroatoms independently selected from O, S, and N, or a 4-7 membered carbocyclic or heterocyclic;
   or U and R¹ together are joined to form an optionally substituted ring selected from a 5 or 6 membered heteroaryl having 1-3 ring heteroatoms independently selected from O, S, and N, or a 4-7 membered carbocyclic or heterocyclic;
   or Z, E, and R³ together are joined to form an optionally substituted ring selected from a 5 or 6 membered heteroaryl having 1-3 ring heteroatoms independently selected from O, S, and N, or a 4-7 membered heterocycle, in this case, the ring atom at the Z or E position is not particularly limited, which for example may be N or C, so long as the ring formed among Z, E, and R³ is an optionally substituted 5 or 6 membered heteroaryl or 4-7 membered heterocycle as defined;
   or R² and R³ together are joined to form an optionally substituted 5-7 membered heterocycle;
   or X and L¹ together are joined to form an optionally substituted fused ring;
   L¹ is an optionally substituted 4-12 membered heterocyclylene having one or more rings and 1-4 ring heteroatoms each independently O, N, or S, or —N(R^{E})—, wherein R^{E} is optionally substituted C₁₋₄ alkyl or optionally substituted 3-7 membered ring;
   L² is absent, O, NH, —N(R^{E2})—, C(0), SO₂, an optionally substituted C₁₋₄ alkylene, an optionally substituted C₂₋₄ alkenylene, an optionally substituted C₁₋₄ alkynylene, optionally substituted C₁₋₄ heteroalkylene, or an optionally substituted 3-7 membered ring, wherein R^{E2} is optionally substituted C₁₋₄ alkyl or optionally substituted 3-7 membered ring; and
   Ring A and R⁴ together represent optionally substituted phenyl, optionally substituted 5 or 6 membered heteroaryl, or optionally substituted 8-12 membered ring having two or more rings;
   provided that when X is N, Z is C(O), Y is N, U is CH, and L¹ is optionally substituted piperazine, then,
   Ring A is an optionally substituted phenylene or heteroarylene; and R⁴ is SR^{F} SF₅, or optionally substituted 5-8 membered carbocyclic having two or more rings, wherein R^{F} is optionally substituted C₁₋₄ alkyl, such as CF₃, or optionally substituted C₃₋₆ cycloalkyl; or
   Ring A and R⁴ together represent an optionally substituted 8-12 membered ring having two or more rings. As customarily used in the art, the “ [symbol] ” as used herein, such as in Formula I, indicates that the bond can be an aromatic bond, a double bond or a single bond as valance permits.

In some embodiments, the compound of Formula I (including any of the applicable sub-formulae as described herein) can have stereoisomer(s). In such embodiments, the compound of Formula I (including any of the applicable sub-formulae as described herein) can exist in the form of an individual enantiomer, diastereomer, atropisomer, and/or geometric isomer, as applicable, or a mixture of stereoisomers, including racemic mixtures and mixtures enriched in one or more stereoisomers. In some embodiments, when applicable, the compound of Formula I (including any of the applicable sub-formulae as described herein) can exist as a mixture of a pair of enantiomers in any ratio, including a racemic mixture with a ratio of 1:1. In some embodiments, when applicable, the compound of Formula I (including any of the applicable sub-formulae as described herein) can exist as an isolated or enriched individual enantiomer substantially free (e.g., with less than 20%, less than 10%, less than 5%, less than 1%, by weight, by HPLC or SFC area, or both, or with a non-detectable amount) of the other enantiomer.

Typically, in Formula I and any of the applicable sub-formulae herein, no more than two (e.g., 0 or 1) of the following pairs (together with the intervening atom(s)) can be joined to form a ring as described above: (1) Y and R¹; (2) U and R¹; (3) Z and R³; (4) R² and R³; or (5) X and L¹. In some embodiments, only one of the foregoing pairs (1)-(5) are joined to form a ring as defined herein. In some embodiments, the following two pairs, (3) Z and R³, and (5) X and L¹, are each joined to form a ring as defined herein.

In some embodiments, the compound of Formula I is characterized as having Ring A being an optionally substituted phenylene or heteroarylene; and R⁴ being SR^{F}, SF₅, or optionally substituted 5-8 membered carbocyclic having two or more rings, wherein R^{F} is optionally substituted C₁₋₄ alkyl, such as CF₃, or optionally substituted C₃₋₆ cycloalkyl. For example, in some embodiments, Ring A can be an optionally substituted phenylene, such as an unsubstituted phenylene, such as or

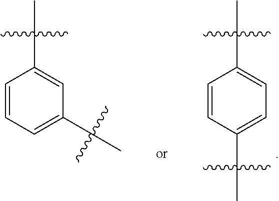 [SMILES: *c1cccc(*)c1 ; *c1ccc(*)cc1]

To be clear, the term “substituted” or “unsubstituted” used in connection with a variable described herein should be understood as referring to whether the variable is substituted, without considering the required bonding of the variable with the remainder of the molecule. For example, an unsubstituted phenylene should be understood such that other than the two ring atoms of the phenylene that are attached to the remainder of the molecule, the other ring atoms of the phenylene are not substituted, whereas in a substituted phenylene, at least one ring atom, other than the two ring atoms of the phenylene that are attached to the remainder of the molecule, is substituted with a substituent described herein. In some embodiments, Ring A can be a substituted phenylene, which for example, can be substituted with one or more (e.g., 1-3) substituents as described herein, for example, each substituent of the phenylene can be independently selected from halogen (e.g., F), OH, CN, C₁₋₄ alkyl or C₁₋₄ alkoxy. In some embodiments, Ring A can be an optionally substituted heteroarylene, such as an optionally substituted 5 or 6-membered heteroarylene having 1-3 ring heteroatoms independently selected from O, N, and S. For example, in some embodiments, Ring A can be an optionally substituted pyridylene or pyrimidinylene, such as unsubstituted pyridylene or pyrimidinylene,

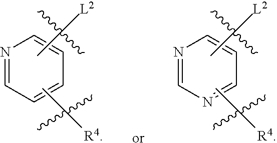

In some embodiments, Ring A can be a pyridylene selected from the following (R⁴ is shown to show direction of attachment of the pyridylene).

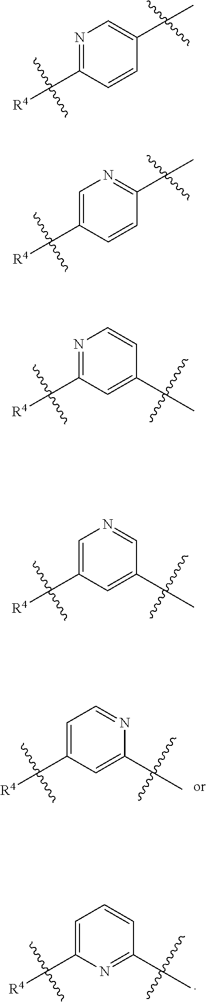

In some embodiments, when Ring A is an optionally substituted phenylene or heteroarylene as described herein, R⁴ can be SR^{F1}, SF₅, or optionally substituted 5-8 membered carbocyclic having two or more rings, such as a bridged bicyclic carbocyclic, wherein R^{F1} is optionally substituted C₁₋₄ alkyl, such as CF₃. For example, in some preferred embodiments, R⁴ can be SR^{F1}, wherein R^{F1} is optionally substituted C₁₋₄ alkyl, such as CF₃. In some preferred embodiments, R⁴ can be SF₅. Based on experimental data, the inventors believe that using SF₅ or SCF₃ group as R⁴ instead of using CF₃ can provide a more balanced DGKalpha and DGKzeta potency profile, better activity in PBMC IL-2 cell assay, and better mouse whole blood 1L2 and IFNgamma activity. Compounds having SF₅ or SCF₃ group as R⁴ instead of CF₃ can also provide overall more advantageous PK profiles.

In some embodiments, R⁴ can be optionally substituted 5-8 membered carbocyclic having two or more rings, such as a bicyclic carbocyclic ring. For example, in some embodiments, R⁴ can be a 5-8 membered bicyclic carbocyclic, such as a bridged bicyclic carbocyclic, such as

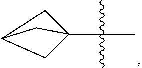 [SMILES: *C12CC(C1)C2]

which is optionally substituted, wherein, when substituted, the bicyclic carbocyclic can be substituted with one or more (e.g., 1-3) substituents, for example, each substituent can be independently selected from halogen (e.g., F), OH, CN, C₁₋₄ alkyl or C₁₋₄ alkoxy. In some embodiments, R⁴ can be

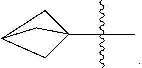 [SMILES: *C12CC(C1)C2]

In some embodiments, the compound of Formula I is characterized in that Ring A and R⁴ together represent an optionally substituted 8-12 membered ring having two or more rings. As used herein, unless specified or otherwise obviously contrary from context, a “x-y membered ring” should be understood as encompassing any ring structure having the designated number of ring members, for example, such ring can be carbocyclic, heterocyclic, aryl or heteroaryl, which can be monocyclic, bicyclic, or having more than two rings, and each of the ring(s) can be saturated, partially unsaturated, or aromatic, and can optionally contain one or more ring heteroatoms. Further, when applicable, such “x-y membered ring” should be understood as attaching to the remainder of the molecule through one or more ring atoms.

In some embodiments, the compound of Formula I is characterized in that Ring A and R⁴ together represent a structure of formula S-1, S-2, S-3, or S-4 below:

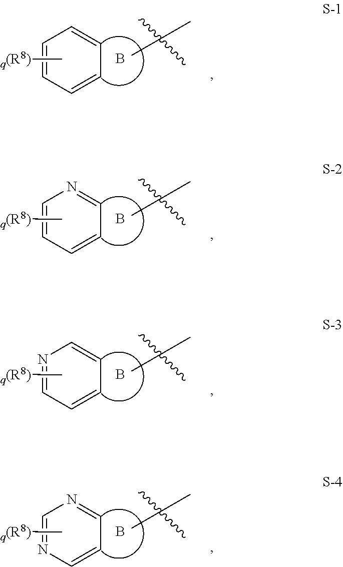

wherein:
   Ring B is attached to L²;
   wherein Ring B is a 5-7 membered ring containing 0, 1, or 2 ring heteroatoms, and is optionally substituted with 1-3 R^{G}, wherein R^{G} at each occurrence is independently halogen, OH, oxo, NH₂, NH(C₁₋₄ alkyl), N(C₁₋₄ alkyl)(C₁₋₄ alkyl), an optionally substituted C₁₋₄ alkyl, or optionally substituted C₁₋₄ alkoxy;
   wherein the phenyl, pyridyl, or pyrimidyl in S-1, S-2, S-3, or S-4 is optionally substituted with q instance(s) of R⁸; wherein:
   q is an integer ranging from 0-3 as valency permits, preferably, 1 or 2,
   and R⁸ at each occurrence is independently halogen (e.g., F, Cl, or Br), CN, OH, NH₂, R^{H}, OR^{H}, NH(R^{H}), N(R^{H})₂, SR^{H}, SF₅, or optionally substituted 5-8 membered carbocyclic having two or more rings, wherein R^{H} at each occurrence is independently optionally substituted C₁₋₄ alkyl, optionally substituted C₂₋₄ alkenyl, optionally substituted C₂₋₄ alkynyl, optionally substituted C₃₋₆ cycloalkyl, or optionally substituted 4-7 membered heterocyclyl;
   wherein, when substituted, the optionally substituted C₁₋₄ alkyl, C₁₋₄ alkoxy, 5-8 membered carbocyclic, C₂₋₄ alkenyl, C₂₋₄ alkynyl, C₃₋₆ cycloalkyl, or 4-7 membered heterocyclyl can be substituted with one or more (e.g., 1-3) substituents, for example, each substituent can be independently selected from halogen (e.g., F), OH, CN, C₁₋₄ alkyl or C₁₋₄ alkoxy. To be clear, the drawing shown in S-1, S-2, S-3, or S-4 should be understood such that R⁸, if present, is to be attached on the phenyl, pyridyl, or pyrimidyl portion of the structure, i.e., not on Ring B. Ring B, if substituted, should be understood as being substituted with 1-3 R^{G} as defined herein.

In some embodiments, Ring B in S-1, S-2, S-3, or S-4 contains no ring heteroatoms. In some embodiments, Ring B in S-1, S-2, S-3, or S-4 contains one ring heteroatom, such as O, N, or S. In some embodiments, Ring B in S-1, S-2, S-3, or S-4 contains no ring heteroatoms and is not substituted. In some embodiments, Ring B in S-1, S-2, S-3, or S-4 contains no ring heteroatoms and is optionally substituted with one or more substituents described herein, for example, in some embodiments, Ring B is optionally substituted with 1-3 R^{G1}, wherein R^{G1} at each occurrence is independently F, OH, C₁₋₄ alkyl optionally substituted with 1-3 F, or C₁₋₄ alkoxy optionally substituted with 1-3 F.

The phenyl, pyridyl, or pyrimidyl portion of S-1, S-2, S-3, or S-4 is typically substituted with 1 R⁸, i.e., q is 1, wherein R⁸ is defined herein. In some embodiments, the phenyl, pyridyl, or pyrimidyl portion of S-1, S-2, S-3, or S-4 can be substituted with 2 R⁸, wherein R⁸ is defined herein. In some embodiments, R⁸ at each occurrence is independently R^{H1}, OR^{H1}, SR^{H1}, SF₅, or optionally substituted 5-8 membered carbocyclic having two or more rings, wherein R^{H1} is C₁₋₄ alkyl optionally substituted with 1-3 F, C₁₋₄ alkoxy optionally substituted with 1-3 F, or C₃₋₆ cycloalkyl optionally substituted with 1-3 substituents independently selected from F, OH, methyl, and methoxy, and wherein when substituted, the optionally substituted 5-8 membered carbocyclic is substituted with 1-3 substituents each independently selected from halogen (e.g., F), OH, CN, C₁₋₄ alkyl and C₁₋₄ alkoxy. In some embodiments, R⁸ can be optionally substituted 5-8 membered carbocyclic having two or more rings, such as a bicyclic carbocyclic ring. For example, in some embodiments, R⁸ can be a 5-8 membered bicyclic carbocyclic, such as a bridged bicyclic carbocyclic, such as

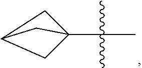 [SMILES: *C12CC(C1)C2]

which is optionally substituted, wherein, when substituted, the bicyclic carbocyclic can be substituted with one or more (e.g., 1-3) substituents, for example, each substituent can be independently selected from halogen (e.g., F), OH, CN, C₁₋₄ alkyl or C₁₋₄ alkoxy. In some embodiments, R⁸ can be

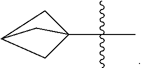 [SMILES: *C12CC(C1)C2]

In some embodiments, R⁸ at each occurrence can be independently a C₁₋₄ alkyl optionally substituted with 1-3 F (such as CH₃, CF₃, etc.), C₁₋₄ alkoxy optionally substituted with 1-3 F (such as OCH₃, OCF₃, etc.), SCF₃, SF₅, cyclopropyl, cyclobutyl, or

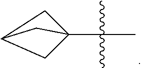 [SMILES: *C12CC(C1)C2]

In some embodiments, the compound of Formula I can be characterized in that Ring A and R⁴ together represent a structure according to formula S-1-A, S-1-B, or S-1-C:

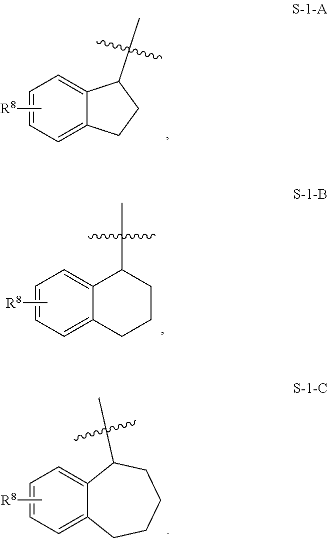

wherein R⁸ can be any of these defined herein. For example, in some embodiments, R⁸ can be independently a C₁₋₄ alkyl optionally substituted with 1-3 F (such as CH₃, CF₃, etc.), C₁₋₄ alkoxy optionally substituted with 1-3 F (such as OCH₃, OCF₃, etc.), SCF₃, SF₅, cyclopropyl, cyclobutyl, or

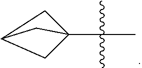 [SMILES: *C12CC(C1)C2]

In some preferred embodiments, Ring A and R⁴ can be such that the compound of Formula I can be characterized in having a structure according to Formula I-1, I-2, I-3, I-4, I-5, or I-6:

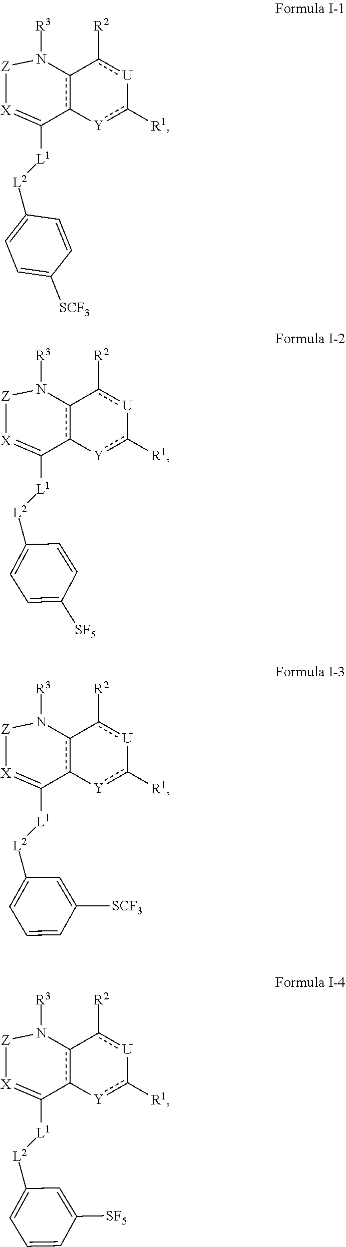

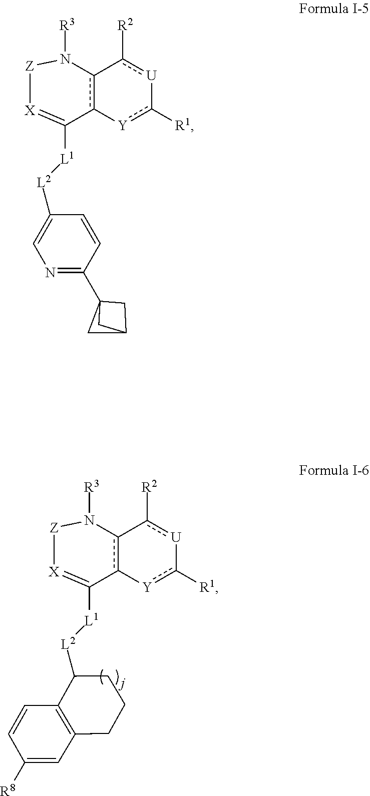

wherein:
R¹, R², R³, U, X, Y, Z, L¹, and L² include any of those respective definitions described and preferred herein in any combinations;
j is 0, 1, or 2, and
R⁸ is halogen (e.g., F, Cl, or Br), CN, OH, NH₂, R^{H}, OR^{H}, NH(R^{H}), N(R^{H})₂, SR^{H}, SF₅, or optionally substituted 5-8 membered carbocyclic having two or more rings, wherein R^{H} at each occurrence is independently optionally substituted C₁₋₄ alkyl, optionally substituted C₂₋₄ alkenyl, optionally substituted C₂₋₄ alkynyl, optionally substituted C₃₋₆ cycloalkyl, or optionally substituted 4-7 membered heterocyclyl;
wherein, when substituted, the optionally substituted 5-8 membered carbocyclic, C₁₋₄ alkyl, C₂₋₄ alkenyl, C₂₋₄ alkynyl, C₃₋₆ cycloalkyl, or 4-7 membered heterocyclyl can be substituted with one or more (e.g., 1-3) substituents, for example, each substituent can be independently selected from halogen (e.g., F), OH, CN, C₁₋₄ alkyl or C₁₋₄ alkoxy. Typically, j in Formula I-6 is 2. In some embodiments, j can also be 0 or 1. In some embodiments, R⁸ is R^{H1}, OR^{H1}, SR^{H1}, SF₅, or optionally substituted 5-8 membered carbocyclic having two or more rings, wherein R^{H1} is C₁₋₄ alkyl optionally substituted with 1-3 F, C₁₋₄ alkoxy optionally substituted with 1-3 F, or C₃₋₆ cycloalkyl optionally substituted with 1-3 substituents independently selected from F, OH, methyl, and methoxy, and wherein when substituted, the optionally substituted 5-8 membered carbocyclic is substituted with 1-3 substituents each independently selected from halogen (e.g., F), OH, CN, C₁₋₄ alkyl and C₁₋₄ alkoxy. In some embodiments, R⁸ can be optionally substituted 5-8 membered carbocyclic having two or more rings, such as a bicyclic carbocyclic ring. For example, in some embodiments, R⁸ can be a 5-8 membered bicyclic carbocyclic, such as a bridged bicyclic carbocyclic, such as

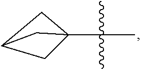 [SMILES: *C12CC(C1)C2]

which is optionally substituted, wherein, when substituted, the bicyclic carbocyclic can be substituted with one or more (e.g., 1-3) substituents, for example, each substituent can be independently selected from halogen (e.g., F), OH, CN, C₁₋₄ alkyl or C₁₋₄ alkoxy. In some embodiments, R⁸ can be

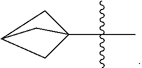 [SMILES: *C12CC(C1)C2]

In some embodiments, R⁸ can be independently a C₁₋₄ alkyl optionally substituted with 1-3 F (such as CH₃, CF₃, etc.), C₁₋₄ alkoxy optionally substituted with 1-3 F (such as OCH₃, OCF₃, etc.), SCF₃, SF₅, cyclopropyl, cyclobutyl, or

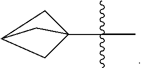 [SMILES: *C12CC(C1)C2]

In some embodiments, in Formula I, subject to the proviso discussed hereinabove, Ring A and R⁴ together can be an optionally substituted phenyl, such as a phenyl which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl. For example, in some embodiments, Ring A and R⁴ together is a phenyl which is substituted with 1-3 (e.g., 1 or 2) R²², wherein R²² at each occurrence is independently halogen (e.g., F, Cl, or Br), CN, OH, R^{M1}, OR^{M1}, SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. More preferably, Ring A and R⁴ together is a phenyl which is substituted with 1-3 (e.g., 1 or 2) R²², wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

In some embodiments, in Formula I, subject to the proviso discussed hereinabove, Ring A and R⁴ together can be optionally substituted 5 or 6-membered heteroaryl, such as a pyridyl

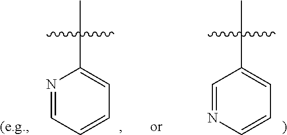 [SMILES: *c1ccccn1 ; *c1cccnc1]

which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl. For example, in some embodiments, Ring A and R⁴ together is a 5-membered heteroaryl

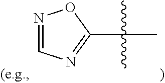 [SMILES: *c1ncno1]

or pyridyl

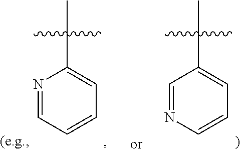 [SMILES: *c1ccccn1 ; *c1cccnc1]

which is substituted with 1-3 (e.g., 1 or 2) R²², as valency permits, wherein R²² at each occurrence is independently halogen (e.g., F, Cl, or Br), CN, OH, R^{M1}, OR^{M1}, SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. More preferably, Ring A and R⁴ together is a 5-membered heteroaryl

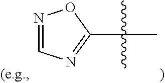 [SMILES: *c1ncno1]

or pyridyl

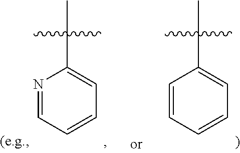 [SMILES: *c1ccccn1 ; *c1ccccc1]

which is substituted with 1-3 (e.g., 1 or 2) R²², as valency permits, wherein R² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

In some embodiments, in Formula I, subject to the proviso discussed hereinabove, Ring A and R⁴ together can be selected from the following:

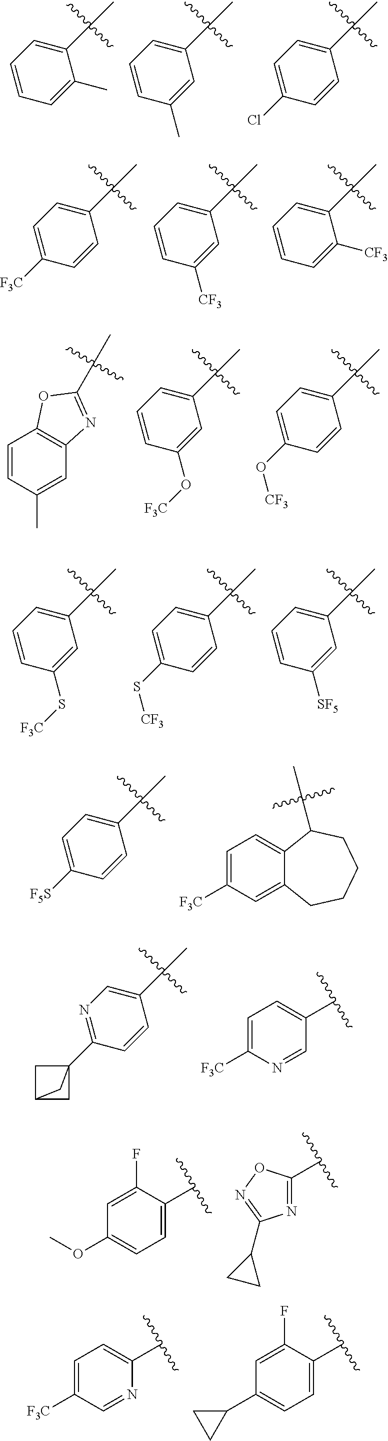

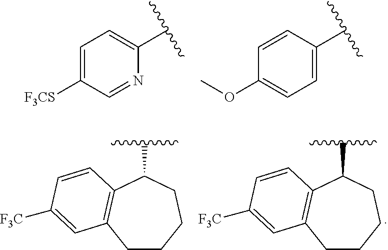

Typically, Y in Formula I (e.g., any of the applicable subformulae, such as I-1, I-2, I-3, I-4, I-5, or I-6) is N.

In some embodiments, Y in Formula I (e.g., any of the applicable subformulae, such as I-1, I-2, I-3, I-4, I-5, or I-6) is CR⁷, wherein R⁷ is hydrogen, F, Cl, CN, optionally substituted C₁₋₄ alkyl, or optionally substituted C₁₋₄ heteroalkyl. For example, in some embodiments, Y is CH.

In some embodiments, Y and R¹ in Formula I (e.g., any of the applicable subformulae, such as I-1, I-2, I-3, I-4, I-5, or I-6) together can be joined to form an optionally substituted ring selected from a 5 or 6 membered heteroaryl ring having 1-3 ring heteroatoms independently selected from O, S, and N, or a 4-7 membered carbocyclic or heterocyclic. For example, in some embodiments, Y and R¹ together can form a 5 or 6 membered heteroaryl having 1-3 ring heteroatoms independently selected from O, S, and N, more preferably, a 5-membered heteroaryl having 1-3 ring nitrogen atoms.

In some preferred embodiments, Y and R¹ together can form a triazole and the compound of Formula I (e.g., any of the applicable subformulae, such as I-1, I-2, I-3, I-4, I-5, or I-6) can be characterized as having Formula I-A:

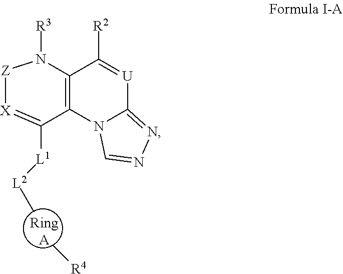

wherein:
R², R³, U, X, Z, L¹, L², Ring A, and R⁴ include any of those respective definitions described and preferred herein in any combinations.

Typically, U in Formula I (e.g., any of the applicable subformulae, such as I-1, I-2, I-3, I-4, I-5, I-6, or I-A) is CR⁶, wherein R⁶ is hydrogen, halogen (e.g., F, Cl, or Br), CN, OH, NH₂, R^{D}, OR^{D}, NH(R^{D}), N(R^{D})₂, COOH, CONH₂, COOR^{D}, CONH(R^{D}), CON(R^{D})₂, SR^{D}, SOR^{D}, SO₂R^{D}, or P(O)(R^{D})₂, wherein R^{D} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl. In some preferred embodiments, R⁶ is hydrogen, i.e., U is CH. In some embodiments, R⁶ can also be halogen, such as F or Cl. In some embodiments, R⁶ can also be OR^{D}, wherein R^{D} is defined herein. In some embodiments, R⁶ can be OR^{D1}, wherein R^{D1} is optionally substituted C₁₋₄ alkyl, optionally substituted C₃₋₆ cycloalkyl, or optionally substituted 4-7 membered heterocyclyl having 1 or 2 ring heteroatoms independently selected from O, N, and S; wherein, when substituted, the optionally substituted C₁₋₄ alkyl, C₃₋₆ cycloalkyl, or 4-7 membered heterocyclyl can be substituted with one or more (e.g., 1-3) substituents, for example, each substituent can be independently selected from halogen (e.g., F), OH, CN, C₁₋₄ alkyl or C₁₋₄ alkoxy. For example, in some embodiments, R⁶ can be C₁₋₄ alkoxy. In some specific embodiments, R⁶ can be

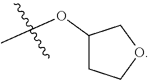 [SMILES: *OC1CCOC1]

In some specific embodiments, R⁶ can be

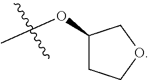 [SMILES: *O[C@@H]1CCOC1]

In some specific embodiments, R⁶ can be

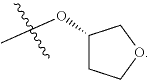 [SMILES: *O[C@H]1CCOC1]

In some embodiments, U in Formula I (e.g., any of the applicable subformulae, such as I-1, I-2, I-3, I-4, I-5, I-6, or I-A) is N.

In some embodiments, U and R¹ in Formula I can also be joined together to form a 5 or 6 membered heteroaryl having 1-3 ring heteroatoms independently selected from O, S, and N, or a 5-7 membered heterocyclyl having 1 or 2 ring heteroatoms independently selected from O, S, and N.

Various groups are suitable as R¹ for Formula I. For example, in some embodiments, R¹ can be OH, COOH, CONH₂, NH₂, R^{A}, OR^{A}, COOR^{A}, NH(R^{A}), N(R^{A})₂, CONH(R^{A}), CON(R^{A})₂, SR^{A}, SOR^{A}, SO₂R^{A}, or P(O)(R^{A})₂, wherein R^{A} is defined herein. In some embodiments, R^{A} at each occurrence can be optionally substituted C₁₋₄ alkyl.

In some preferred embodiments, R¹ in Formula I (e.g., any of the applicable subformulae, such as I-1, I-2, I-3, I-4, I-5, or I-6) is CN.

In some preferred embodiments, R¹ in Formula I (e.g., any of the applicable subformulae, such as I-1, I-2, I-3, I-4, I-5, or I-6) is halogen, preferably F or Cl.

In some embodiments, R¹ in Formula I (e.g., any of the applicable subformulae, such as I-1, I-2, I-3, I-4, I-5, or I-6) is a C₂₋₃ alkynyl, such as

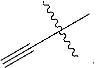 [SMILES: *C#C]

In some embodiments, R¹ in Formula I (e.g., any of the applicable subformulae, such as I-1, I-2, I-3, I-4, I-5, or I-6) is optionally substituted 5 or 6 membered heteroaryl having 1-4 ring heteroatoms independently selected from O, S, and N. For example, in some embodiments, R¹ can be an optionally substituted pyrimidinyl or an optionally substituted thiazolyl, for example, R¹ is

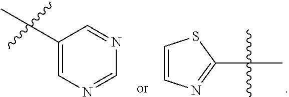 [SMILES: *c1cncnc1 ; *c1nccs1]

In some embodiments, R¹ in Formula I (e.g., any of the applicable subformulae, such as I-1, I-2, I-3, I-4, I-5, or I-6) is SR^{A}, wherein R^{A} is defined herein. For example, in some embodiments, R¹ can be SR^{A1}, wherein R^{A1} is independently optionally substituted C₁₋₄ alkyl, for example, R¹ is SCH₃. Other suitable R¹ for Formula I are described herein.

Typically, R² in Formula I (e.g., any of the applicable subformulae, such as I-1, I-2, I-3, I-4, I-5, I-6, or I-A) is hydrogen. In some embodiments, R² can be halogen (e.g., F, Cl, or Br), CN, OH, NH₂, R^{B}, OR^{B}, NH(R^{B}), or N(R^{B})₂, wherein R^{B} is defined herein. In some embodiments, R^{B} can be an optionally substituted C₁₋₄ alkyl or optionally substituted 3-4 membered cycloalkyl.

Typically, R³ in Formula I (e.g., any of the applicable subformulae, such as I-1, I-2, I-3, I-4, I-5, I-6, or I-A) is an optionally substituted C₁₋₄ alkyl, such as CH₃, CD₃ etc. In preferred embodiments, R³ is CH₃. In some embodiments, R³ can also be an optionally substituted C₃₋₆ cycloalkyl.

In some embodiments, R² and R³ in Formula I (e.g., any of the applicable subformulae, such as I-1, I-2, I-3, I-4, I-5, or I-6) can be joined together to form a 5-7 membered heterocycle having 0, 1, or 2 ring heteroatoms selected from O, N, and S, in addition to the nitrogen atom to which R³ is attached. For example, in some embodiments, the compound of Formula I (e.g., any of the applicable subformulae, such as I-1, I-2, I-3, I-4, I-5, or I-6) can be characterized as having a structure according to Formula I-B:

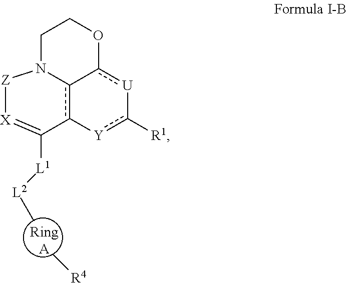

wherein:
R¹, U, X, Y, Z, L¹, L², Ring A, and R⁴ include any of those respective definitions described and preferred herein in any combinations.

Typically, Z in Formula I (e.g., any of the applicable subformulae, such as I-1, I-2, 1-3, 1-4, 1-5, 1-6, I-A, or I-B) is C(O).

In some embodiments, Z in Formula I (e.g., any of the applicable subformulae, such as I-1, I-2, I-3, I-4, I-5, I-6, I-A, or I-B) can also be S(O)₂.

In some preferred embodiments, Z and R³ in Formula I (e.g., any of the applicable subformulae, such as I-1, I-2, I-3, I-4, I-5, or I-6) can also be joined together to form a 5 or 6 membered heteroaryl having 1-3 ring heteroatoms independently selected from O, S, and N. For example, in some embodiments, Z and R³ together form a 5-membered heteroaryl having 1-3 (preferably 2 or 3) ring nitrogen atoms. In some embodiments, the compound of Formula I (e.g., any of the applicable subformulae, such as I-1, I-2, I-3, I-4, I-5, or I-6) can be characterized as having a structure according to Formula I-C:

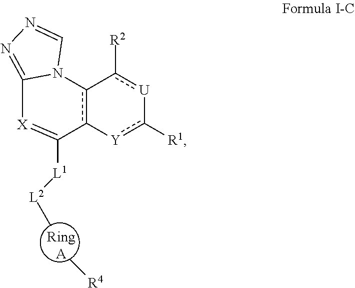

wherein:
R¹, R², U, X, Y, L¹, L², Ring A, and R⁴ include any of those respective definitions described and preferred herein in any combinations.

In some embodiments, X in Formula I (e.g., any of the applicable subformulae, such as I-1, I-2, I-3, I-4, I-5, I-6, I-A, I-B, or I-C) is N.

In some embodiments, X in Formula I (e.g., any of the applicable subformulae, such as I-1, I-2, I-3, I-4, I-5, I-6, I-A, I-B, or I-C) is CR⁵, wherein R⁵ is defined herein. In some embodiments, X in Formula I (e.g., any of the applicable subformulae, such as I-1, I-2, I-3, I-4, I-5, I-6, I-A, I-B, or I-C) is CR⁵, wherein R⁵ is CN. In some embodiments, X in Formula I (e.g., any of the applicable subformulae, such as I-1, I-2, I-3, I-4, I-5, I-6, I-A, I-B, or I-C) is CR⁵, wherein R⁵ is CONH₂, COOH, CONH(R^{C1}), CON(R^{C1})₂, or COOR^{C1}, wherein R¹ at each occurrence is independently a C₁₋₄ alkyl, such as methyl.

In some embodiments, X in Formula I (e.g., any of the applicable subformulae, such as I-1, I-2, I-3, I-4, I-5, or I-6) is CR⁵, wherein R⁵ is joined with L¹ to form an optionally substituted fused ring.

In some preferred embodiments, the compound of Formula I (e.g., any of the applicable subformulae, such as I-1, I-2, I-3, I-4, I-5, I-6, I-A, I-B, or I-C) can be characterized as having a structure according to Formula I-A-1, I-B-1, I-C-1, I-D, or I-E:

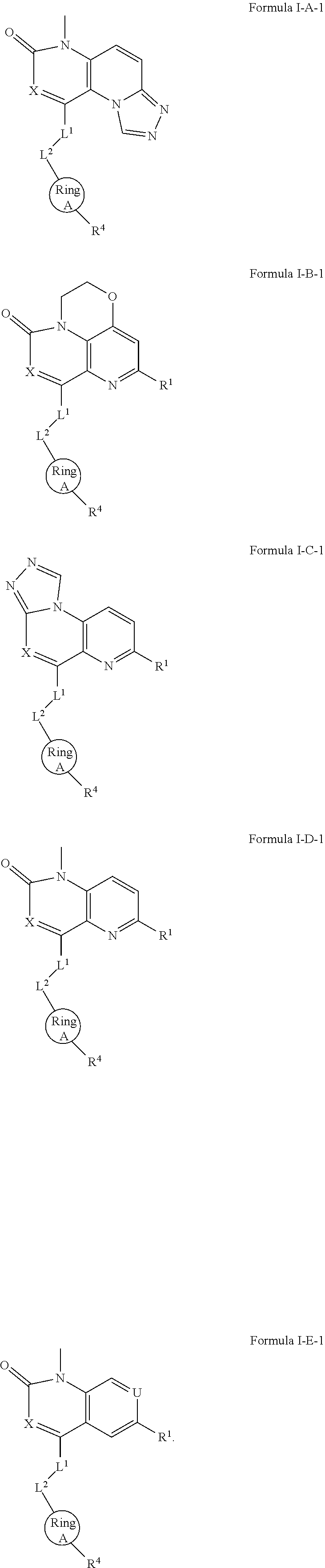

wherein:
R¹, U, X, L¹, L², Ring A, and R⁴ include any of those respective definitions described and preferred herein in any combinations.

In some embodiments, L¹ in Formula I is an optionally substituted 4-12 membered heterocyclylene having one or more rings and 1-4 ring heteroatoms each independently O, N, or S. In some embodiments, L¹ in Formula I can be —N(R^{E})—, wherein R^{E} is optionally substituted C₁₋₄ alkyl or optionally substituted 3-7 membered ring. For example, in some embodiments, L¹ is —N(C₁₋₄ alkyl)-, such as —N(CH₃)—.

In some embodiments, L¹ in Formula I (e.g., any of the applicable subformulae, such as I-1, I-2, I-3, I-4, I-5, I-6, I-A, I-B, I-C, I-A-1, I-B-1, I-C-1, I-D, or I-E) is an optionally substituted 4-7 membered monocyclic heterocyclylene having 1 or 2 ring heteroatoms each independently O, N, or S. In some embodiments, the 4-7 membered monocyclic heterocyclylene is a saturated heterocyclylene having 1 or 2 ring heteroatoms, such as 1 or 2 ring nitrogen atoms, such as a piperazine or piperidine ring. The heterocyclylene is typically attached to the remainder of the molecule through two ring nitrogen atoms or one ring nitrogen and one ring carbon atom. For example, in some embodiments, L¹ can be

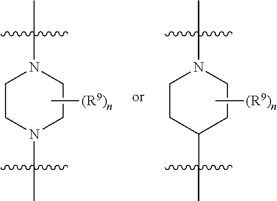

(either of the two attaching points of the piperidine, N or C, can be attached to L²),
wherein:
n is 0, 1, 2, 3, or 4, and
   (1) R⁹ at each occurrence is independently R^{N}, COOR^{N}, COR^{N}, CONH(R^{N}), or CON(R^{N})₂, wherein R^{N} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, or optionally substituted 5 or 6 membered heteroaryl;
   (2) two instances of R⁹ can be joined to form a double bond or a 3-5 membered ring, and any remaining instance(s) of R⁹ are as defined in (1); or
   (3) when X is CR⁵, one instance of R⁹ and R⁵ can be joined to form a 5-7 membered ring and any remaining instance(s) of R⁹ are as defined in (1).

Typically, n is 0, 1, or 2. For example, L¹ can be selected from:

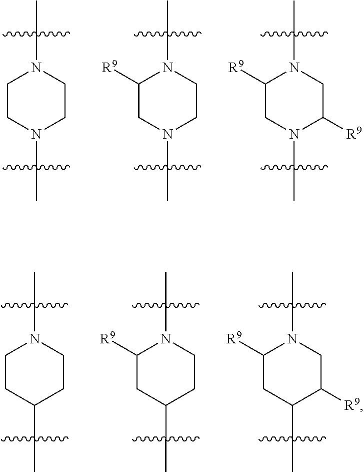

wherein either of the two attaching points can be attached to L². In some embodiments, R⁹ at each occurrence can be independently CH₃, CH₂CH₃, CH₂CH₂CH₃, fluorine substituted C₁₋₃ alkyl (e.g., CF₂H), cyclopropyl, cyclobutyl, CH₂OH, CH₂OCH₃, CH₂OCH₂CH₃, CH₂NH₂, CH₂N₃, or CH₂NHC(O)OCH₃. In some embodiments, wherein X is CR⁵, one instance of R⁹ is joined with R⁵, together with the intervening atoms, to form a 5-7 membered ring, and the other R⁹ group(s), if present, are as defined herein. For example, in some embodiments, the compound of Formula I can be characterized as having a structure according to Formula I-D-1 or I-D-2:

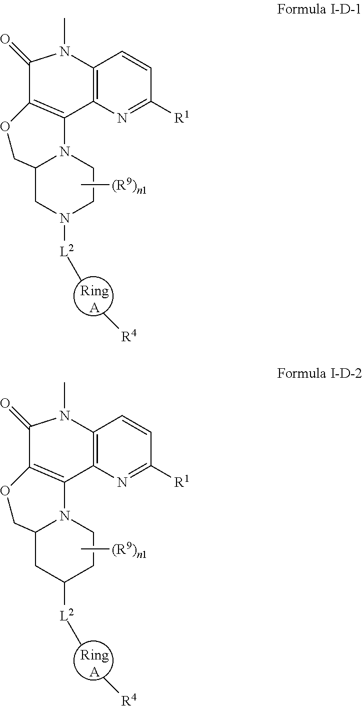

wherein:
R¹, R⁹, L², Ring A, and R⁴ include any of those respective definitions described and preferred herein in any combinations, and
n1 is 0, 1, 2, or 3. Typically, n1 is 0 or 1.

In some preferred embodiments, L¹ in Formula I (e.g., any of the applicable subformulae, such as I-1, I-2, I-3, I-4, I-5, I-6, I-A, I-B, I-C, I-A-1, I-B-1, I-C-1, I-D, or I-E) can be selected from the following (the bottom attaching point, N or C, is attached to L²)

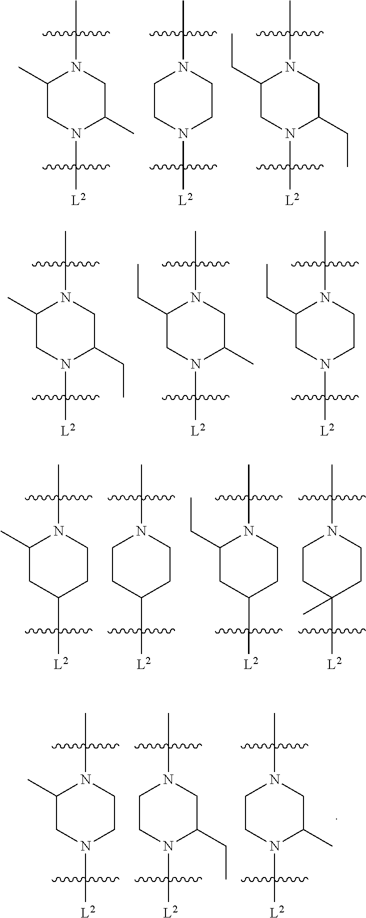

In some preferred embodiments, L¹ in Formula I (e.g., any of the applicable subformulae, such as I-1, I-2, I-3, I-4, I-5, I-6, I-A, I-B, I-C, I-A-1, I-B-1, I-C-1, I-D, or I-E) can be selected from the following (the bottom attaching point, N or C, is attached to L²):

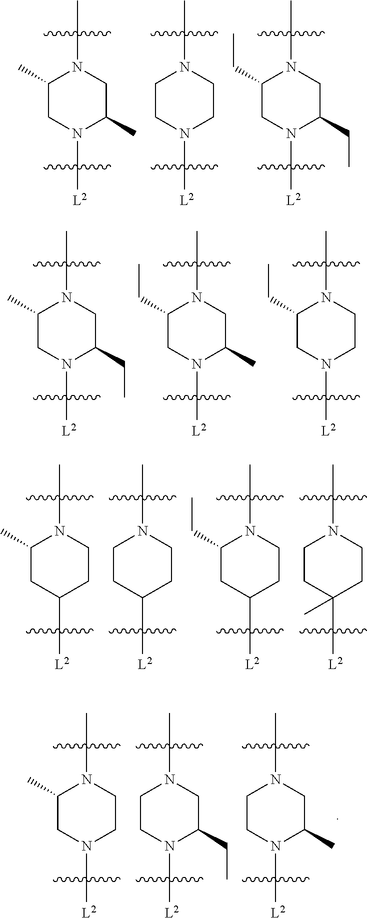

In some embodiments, L in Formula I (e.g., any of the applicable subformulae, such as I-1. I-2,I-3,I-4,I-5,I-6, In e-B. I-C. I-A-1 I-B-1. I-C-1 I-D. or I-E) can also be an optionally substituted 7-12 membered heterocyclylene having two or more rings and 1-4 ring heteroatoms each independently O, N, or S, when substituted, the substituent(s) can be attached to any one or more of the two or more rings. For example, in some embodiments, L¹ can be an optionally substituted 8-12 membered fused, spiro, or bridged bicyclic heterocyclylene having 1-4 ring heteroatoms each independent O, N, or S, wherein each ring of the bicyclic heterocyclylene can be saturated, partially unsaturated, or aromatic, and each ring of the bicyclic heterocyclylene can have 0, 1, 2, or 3 ring heteroatoms, provided that the bicyclic heterocyclylene as a whole is not fully aromatic and the total number of heteroatoms in the bicyclic heterocyclylene does not exceed 4. In some embodiments, L¹ can be an optionally substituted 8-11 membered fused bicyclic heterocyclylene having 1-3 ring heteroatoms each independent O, N, or S, wherein (1) one of the two fused rings is phenyl or 5 or 6 membered heteroaryl, and (2) the other of the two fused rings is a 5-7 membered heterocycle having one or two ring heteroatoms each independently O, N, or S, preferably, the 5-7 membered heterocycle has at least one ring nitrogen atom. In some embodiments, L¹ can be an optionally substituted 8-11 (e.g., 8, 9, or 10) membered spiro bicyclic heterocyclylene having 1-4 ring heteroatoms each independent O, N, or S, wherein (1) one of the two spiro rings is a 5-7 membered heterocycle having one or two ring heteroatoms each independently O, N, or S, preferably, the 5-7 membered heterocycle has one ring nitrogen atom, and (2) the other of the two spiro rings is a 4-6 membered heterocycle having 1-3 ring heteroatoms each independently O, N, or S, for example, one of the two spiro rings is a pyrrolidine, piperidine, azepane ring and the other of the two spiro rings is azetidine, pyrrolidine, pyrrolidinone, piperidinone, oxazoline, isoxazoline, thiazoline, isothiazoline, etc.

For example, in some preferred embodiments, L¹ in Formula I (e.g., any of the applicable subformulae, such as I-1, I-2, I-3, I-4, I-5, I-6, I-A, I-B, I-C, I-A-1, I-B-1, I-C-1, I-D, or I-E) can be selected from the following (the bottom attaching point, N or C, is attached to L²):

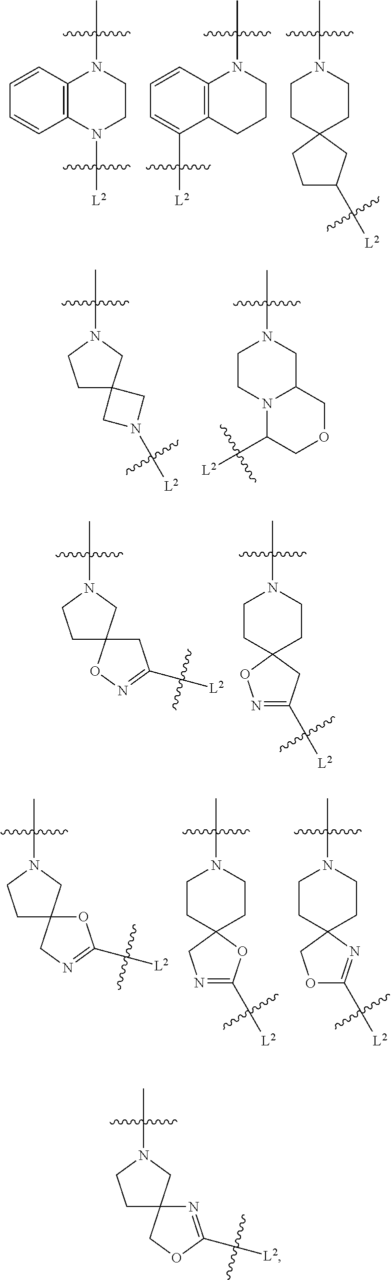

wherein in each of the bicyclic heterocyclylene, each of the two rings can be optionally substituted with 1-3 R¹⁰, wherein R¹⁰ at each occurrence is independently halogen, OH, NH₂, oxo (as applicable), R^{J}, OR^{J}, CN, NH(R^{J}), or N(R^{J})₂, wherein R^{J} at each occurrence is independently an optionally substituted C₁₋₄ alkyl, optionally substituted C₂₋₄ alkenyl, optionally substituted C₂₋₄ alkynyl, or optionally substituted 3-4 membered ring.

In some preferred embodiments, L¹ in Formula I (e.g., any of the applicable subformulae, such as I-1, I-2, I-3, I-4, I-5, I-6, I-A, I-B, I-C, I-A-1, I-B-1, I-C-1, I-D, or I-E) can be selected from the following (the bottom attaching point, N or C, is attached to L²):

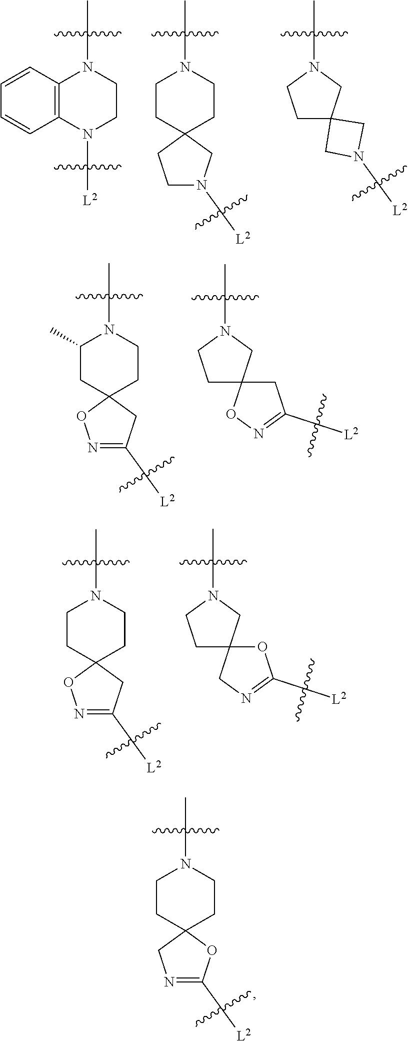

or L¹ is selected from

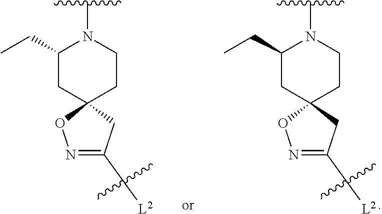

In some embodiments, L in Formula I (e.g., any of the applicable subformulae, such as I-1, I-2, I-3, I-4, I-5, I-6, I-A, I-B, I-C, I-A-1, I-B-1, I-C-1, I-D, or I-E) can be absent.

In some embodiments, L² in Formula I (e.g., any of the applicable subformulae, such as I-1, I-2, I-3, I-4, I-5, I-6, I-A, I-B, I-C, I-A-1, I-B-1, I-C-1, I-D, or I-E) can be O or C(O).

In some embodiments, L² in Formula I (e.g., any of the applicable subformulae, such as I-1, I-2, I-3, I-4, I-5, I-6, I-A, I-B, I-C, I-A-1, I-B-1, I-C-1, I-D, or I-E) can be an optionally substituted C₁₋₄ alkylene, wherein, when substituted, the C₁₋₄ alkylene is substituted with 1-3 R¹¹, wherein R¹¹ at each occurrence is independently selected from halogen (e.g., F, Cl, or Br), CN, OH, NH₂, COOH, CONH₂, SO₂NH₂, COR^{K}, COOR^{K}, CONH(R^{K}), CON(R^{K})₂, SO₂R^{K}, SO₂NH(R^{K}), SO₂N(R^{K})₂, R^{K}, OR^{K}, NH(R^{K}), N(R^{K})₂, SR^{K}, SOR^{K}, SO₂R^{K}, or P(O)(R^{K})₂, wherein R^{K} at each occurrence is independently optionally substituted C₁₋₄ alkyl, optionally substituted C₂₋₄ alkenyl, optionally substituted C₂₋₄ alkynyl, optionally substituted C₁₋₄ heteroalkyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl.

In some embodiments, L² in Formula I (e.g., any of the applicable subformulae, such as I-1, I-2, I-3, I-4, I-5, I-6, I-A, I-B, I-C, I-A-1, I-B-1, I-C-1, I-D, or I-E) can be an unsubstituted C₁₋₄ alkylene such as CH2, CH(CH3), CH(C2H5), CH(C3H7), etc.

In some embodiments, L² in Formula I (e.g., any of the applicable subformulae, such as I-1, I-2, I-3, I-4, I-5, I-6, I-A, I-B, I-C, I-A-1, I-B-1, I-C-1, I-D, or I-E) can be an unsubstituted C₁₋₄ alkylene selected from: CH₂, or

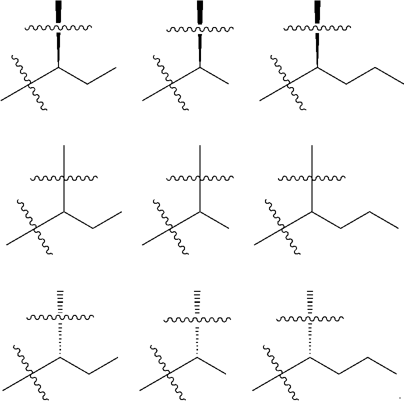

In some embodiments, L² in Formula I (e.g., any of the applicable subformulae, such as I-1, I-2, I-3, I-4, I-5, I-6, I-A, I-B, I-C, I-A-1, I-B-1, I-C-1, I-D, or I-E) can be a C₁₋₄ alkylene substituted with R¹², wherein R¹² is defined herein. For example, in some embodiments, L² can be an R¹²-substituted C₁₋₄ alkylene selected from the following:

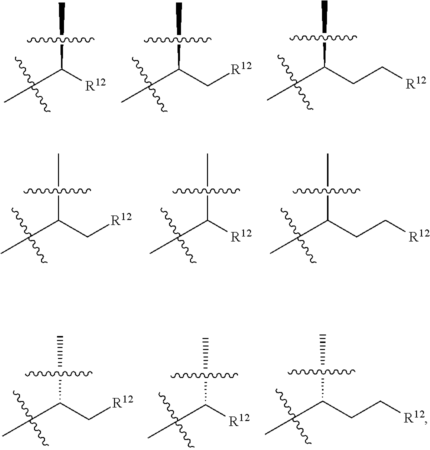

wherein:
R¹² is selected from halogen (e.g., F, Cl, or Br), CN, OH, NH₂, COOH, CONH₂, SO₂NH₂, COR^{K}, COOR^{K}, CONH(R^{K}), CON(R^{K})₂, SO₂R^{K}, SO₂NH(R^{K}), SO₂N(R^{K})₂, R^{K}, OR^{K}, NH(R^{K}), N(R^{K})₂, SR^{K}, SOR^{K}, SO₂R^{K}, or P(O)(R^{K})₂, wherein R^{K} at each occurrence is independently optionally substituted C₁₋₄ alkyl, optionally substituted C₂₋₄ alkenyl, optionally substituted C₂₋₄ alkynyl, optionally substituted C₁₋₄ heteroalkyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl,
wherein, when substituted, the optionally substituted C₁₋₄ alkyl, C₂₋₄ alkenyl, C₂₋₄ alkynyl, C₁₋₄ heteroalkyl, C₃₋₆ cycloalkyl, phenyl, 5 or 6 membered heteroaryl, or 4-7 membered heterocyclyl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. In some embodiments, R¹² can be CN, OH, COOH, CONH₂, methoxy, ethoxy, cyclopropyl, cyclobutyl, optionally substituted phenyl, or optionally substituted 5 or 6 membered heteroaryl having 1-3 ring heteroatoms independently selected from N, O, and S, wherein, when substituted, the optionally substituted phenyl or 5 or 6 membered heteroaryl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. In some embodiments, R¹² is a 3-4 membered ring, such as cyclopropyl, cyclobutyl, oxetanyl, azetidinyl, etc., which is optionally substituted, such as with F and/or methyl. For example, in some embodiments, R¹² is cyclopropyl. In some embodiments, R¹² is an optionally substituted phenyl or optionally substituted 5 or 6 membered heteroaryl having 1-3 ring heteroatoms independently selected from N, O, and S, such as an optionally substituted phenyl or thiazole, which is optionally substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. For example, in some embodiments, R¹² is

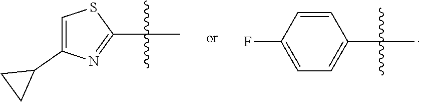 [SMILES: *c1nc(C2CC2)cs1 ; *c1ccc(F)cc1]

In some embodiments, L² in Formula I (e.g., any of the applicable subformulae, such as I-1, I-2, I-3, I-4, I-5, I-6, I-A, I-B, I-C, I-A-1, I-B-1, I-C-1, I-D, or I-E) can be an optionally substituted 5 or 6 membered heteroarylene, for example,

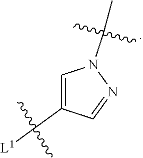

In some embodiments, L² in Formula I (e.g., any of the applicable subformulae, such as I-1, I-2, I-3, I-4, I-5, I-6, I-A, I-B, I-C, I-A-1, I-B-1, I-C-1, I-D, or I-E) can be an optionally substituted phenylene, e.g.,

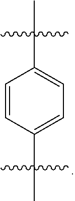 [SMILES: *c1ccc(*)cc1]

In some embodiments, L¹ and L² are selected such that the compound of Formula I (e.g., any of the applicable subformulae, such as I-1, I-2, I-3, I-4, I-5, I-6, I-A, I-B, I-C, I-A-1, I-B-1, I-C-1, I-D, or I-E) can be characterized as having a structure according to Formula I-L-1, I-L-2, I-L-3, I-L-4, I-L-5, I-L-6, I-L-7, I-L-8, I-L-9, I-L-10, or I-L-11:

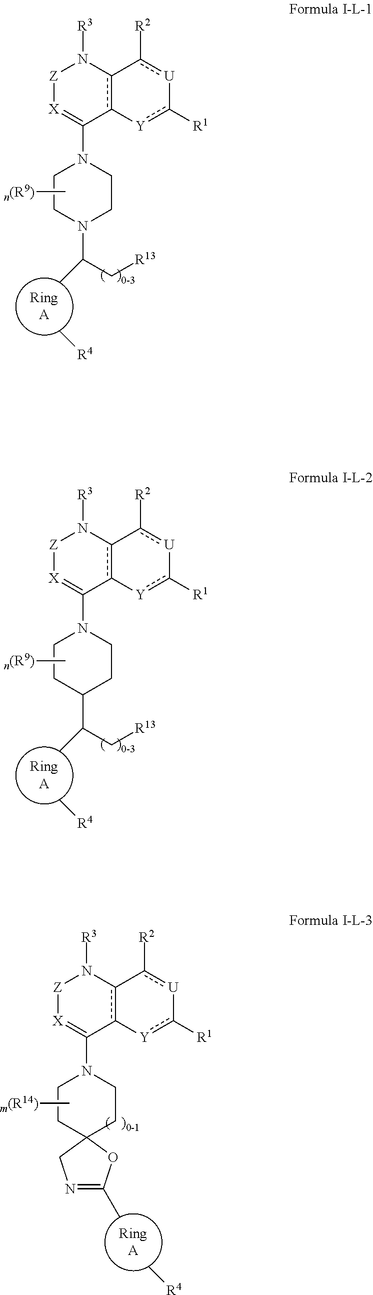

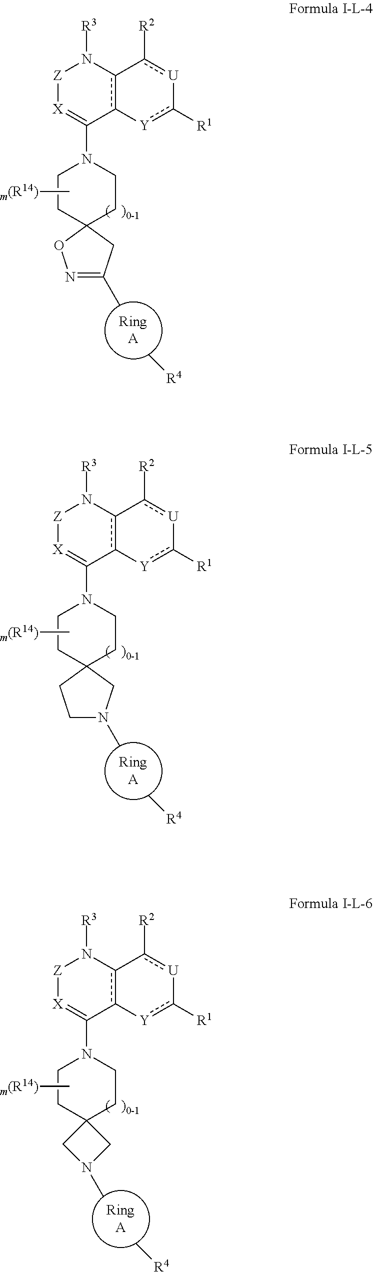

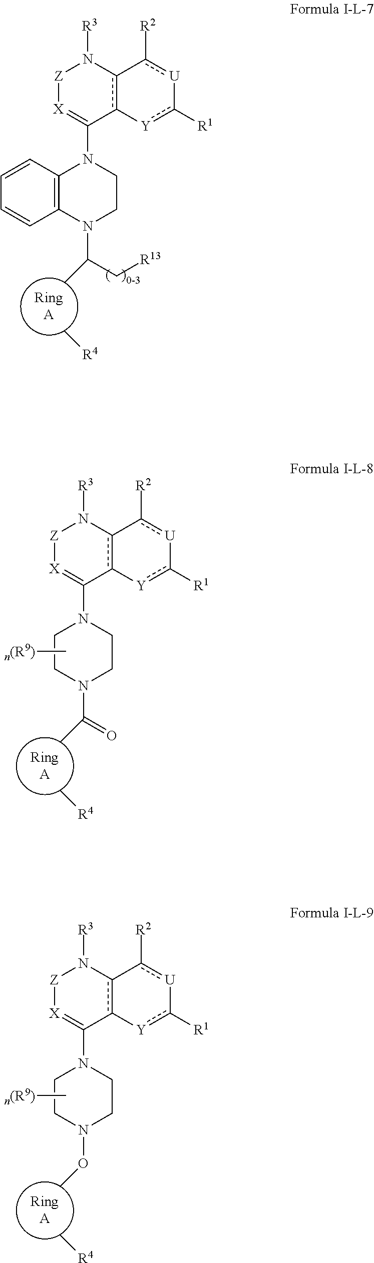

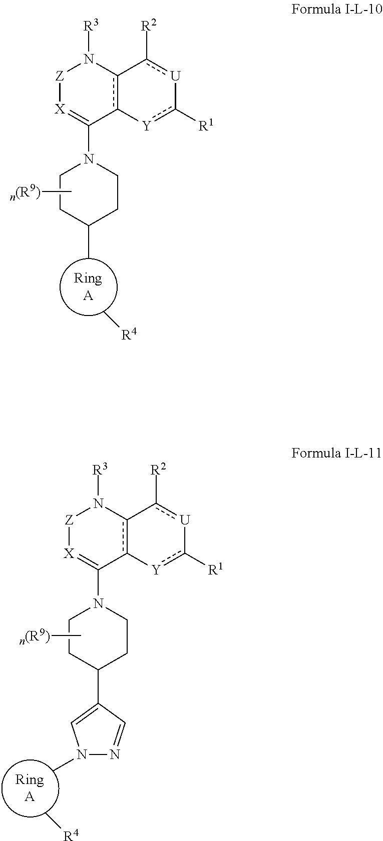

wherein:
   X, Y, U, Z, R¹, R², R³, R⁴, and Ring A include any of those respective definitions described and preferred herein in any combinations,
   R⁹ at each occurrence is independently CH₃, CH₂CH₃, CH₂CH₂CH₃, fluorine substituted C₁₋₃ alkyl (e.g., CF₂H), cyclopropyl, cyclobutyl, CH₂OH, CH₂OCH₃, CH₂OCH₂CH₃, CH₂NH₂, CH₂N₃, or CH₂NHC(O)OCH₃;
   n is 0, 1, 2, or 3;
   R¹³ is selected from hydrogen, halogen (e.g., F, Cl, or Br), CN, OH, NH₂, COOH, CONH₂, SO₂NH₂, COR^{K}, COOR^{K}, CONH(R^{K}), CON(R^{K})₂, SO₂R^{K}, SO₂NH(R^{K}), SO₂N(R^{K})₂, R^{K}, OR^{K}, NH(R^{K}), N(R^{K})₂, SR^{K}, SOR^{K}, SO₂R^{K}, or P(O)(R^{K})₂, wherein R^{K} at each occurrence is independently optionally substituted C₁₋₄ alkyl, optionally substituted C₂₋₄ alkenyl, optionally substituted C₂₋₄ alkynyl, optionally substituted C₁₋₄ heteroalkyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl;
   or one instance of R⁹ and R¹³ together with the intervening atom(s) form an optionally substituted 3-7 membered ring, and the other instance(s) of R⁹ at each occurrence is independently CH₃, CH₂CH₃, CH₂CH₂CH₃, fluorine substituted C₁₋₃ alkyl (e.g., CF₂H), cyclopropyl, cyclobutyl, CH₂OH, CH₂OCH₃, CH₂OCH₂CH₃, CH₂NH₂, CH₂N₃, or CH₂NHC(O)OCH₃;
   R¹⁴ at each occurrence is independently an optionally substituted C₁₋₄ alkyl; and
   m is 0, 1, or 2;
wherein, when substituted, the optionally substituted C₁₋₄ alkyl, C₂₋₄ alkenyl, C₂₋₄ alkynyl, C₁₋₄ heteroalkyl, C₃₋₆ cycloalkyl, phenyl, 5 or 6 membered heteroaryl, 4-7 membered heterocyclyl, or 3-7 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. In some embodiments, R¹³ is hydrogen. In some embodiments, R¹³ is hydrogen, CN, OH, COOH, CONH₂, methoxy, ethoxy, cyclopropyl, cyclobutyl, optionally substituted phenyl, or optionally substituted 5 or 6 membered heteroaryl having 1-3 ring heteroatoms independently selected from N, O, and S, wherein, when substituted, the optionally substituted phenyl or 5 or 6 membered heteroaryl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. In some embodiments, m is 0. In some embodiments, m is 1 and R¹⁴ is C₁₋₄ alkyl such as methyl. In some embodiments, n is 0. In some embodiments, n is 1 or 2 and R⁹ at each occurrence is independently CH₃, CH₂CH₃, CH₂CH₂CH₃, or fluorine substituted C₁₋₃ alkyl (e.g., CF₂H). In some embodiments, R¹³ is a 3-4 membered ring, such as cyclopropyl, cyclobutyl, oxetanyl, azetidinyl, etc., which is optionally substituted, such as with F and/or methyl. For example, in some embodiments, R¹³ is cyclopropyl. In some embodiments, R¹³ is an optionally substituted phenyl or optionally substituted 5 or 6 membered heteroaryl having 1-3 ring heteroatoms independently selected from N, O, and S, such as an optionally substituted phenyl or thiazole, which is optionally substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. For example, in some embodiments, R¹³ is

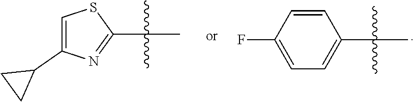 [SMILES: *c1nc(C2CC2)cs1 ; *c1ccc(F)cc1]

### Formula II

In some embodiments, the present disclosure provides a compound of Formula II-1, II-2, II-3, II-4, II-5, II-6, or II-7, or a pharmaceutically acceptable salt thereof:

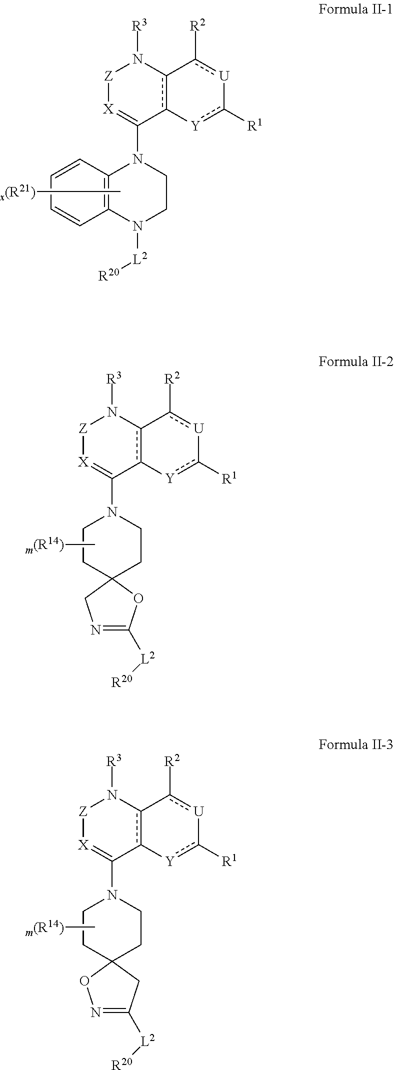

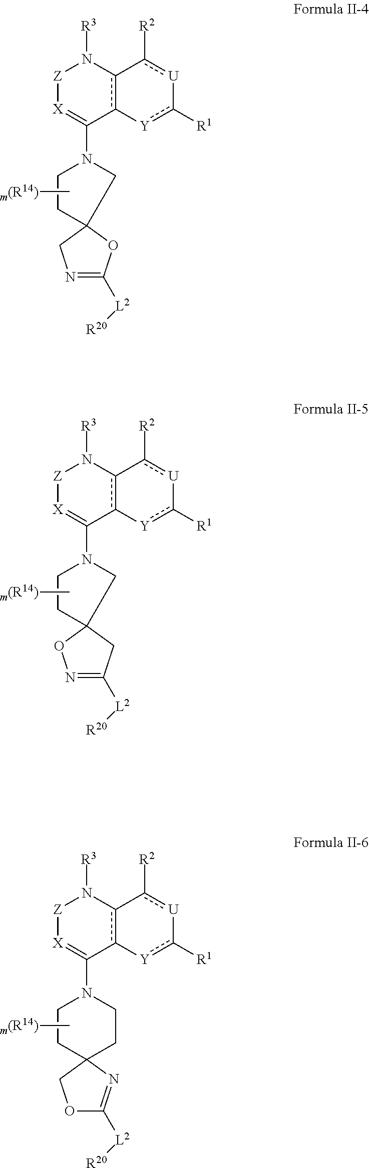

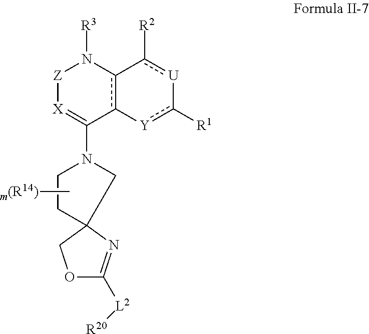

wherein:
   R¹ is hydrogen, halogen (e.g., F, Cl, or Br), CN, OH, COOH, CONH₂, NH₂, R^{A}, OR^{A}, NH(R^{A}), N(R^{A})₂, COOR^{A}, CONH(R^{A}), CON(R^{A})₂, SR^{A}, SOR^{A}, SO₂R^{A}, or P(O)(R^{A})₂, wherein R^{A} at each occurrence is independently optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
   R² is hydrogen, halogen (e.g., F, Cl, or Br), CN, OH, NH₂, R^{B}, OR^{B}, NH(R^{B}), or N(R^{B})₂, wherein R^{B} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, or optionally substituted 4-7 membered heterocyclyl;
   R³ is hydrogen, optionally substituted C₁₋₄ alkyl, optionally substituted C₃₋₆ cycloalkyl, or optionally substituted 4-7 membered heterocyclyl (such as a saturated 4-7 membered heterocyclyl);
   X is N or CR⁵, wherein R⁵ is hydrogen, halogen, CN, CONH₂, COOH, CONH(R^{C}), CON(R^{C})₂, OR^{C}, NO₂, or COOR^{C}, wherein R^{C} at each occurrence is independently an optionally substituted C₁₋₄ alkyl;
   U is N or CR⁶, wherein R⁶ is hydrogen, halogen (e.g., F, Cl, or Br), CN, OH, NH₂, R^{D}, OR^{D}, NH(R^{D}), N(R^{D})₂, COOH, CONH₂, COOR^{D}, CONH(R^{D}), CON(R^{D})₂, SR^{D}, SOR^{D}, SO₂R^{D}, or P(O)(R^{D})₂, wherein R^{D} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
   Y is N or CR⁷, wherein R⁷ is hydrogen, F, Cl, CN, optionally substituted C₁₋₄ alkyl, or optionally substituted C₁₋₄ heteroalkyl;
   Z is C(O), C(═N—OH), C(═N—O—(C₁₋₄ alkyl)), C(═N—NH₂), C(═N—NH—(C₁₋₄ alkyl)), C(═N—N(C₁₋₄ alkyl)(C₁₋₄ alkyl)), SO, SO₂, S(O)(═NH), or S(O)(═N—(C₁₋₄ alkyl);
   or Y and R¹ together are joined to form an optionally substituted ring selected from a 5 or 6 membered heteroaryl having 1-3 ring heteroatoms independently selected from O, S, and N, or a 4-7 membered carbocyclic or heterocyclic;
   or U and R¹ together are joined to form an optionally substituted ring selected from a 5 or 6 membered heteroaryl having 1-3 ring heteroatoms independently selected from O, S, and N, or a 4-7 membered carbocyclic or heterocyclic;
   or Z and R³ together are joined to form an optionally substituted ring selected from a 5 or 6 membered heteroaryl having 1-3 ring heteroatoms independently selected from O, S, and N, or a 4-7 membered heterocycle;
   or R² and R³ together are joined to form an optionally substituted ring selected from a 5-7 membered heterocycle;
   L² is absent, O, NH, —N(R^{E2})—, C(0), SO₂, an optionally substituted C₁₋₄ alkylene, an optionally substituted C₂₋₄ alkenylene, an optionally substituted C₁₋₄ alkynylene, optionally substituted C₁₋₄ heteroalkylene, or an optionally substituted 3-7 membered ring, wherein R^{E2} is optionally substituted C₁₋₄ alkyl or optionally substituted 3-7 membered ring;
   R²⁰ is an optionally substituted phenyl, optionally substituted 5 or 6 membered heteroaryl, or optionally substituted 8-12 membered ring having two or more rings;
   R¹⁴ at each occurrence is independently an optionally substituted C₁₋₄ alkyl;
   m is 0, 1, or 2;
   R²¹ at each occurrence is independently halogen (e.g., F, Cl, or Br), CN, OH, R^{L} OR^{L}, wherein R^{L} at each occurrence is independently an optionally substituted C₁₋₄ alkyl, optionally substituted C₂₋₄ alkenyl, optionally substituted C₂₋₄ alkynyl, or optionally substituted C₃₋₆ cycloalkyl; and
   x is 0, 1, 2, or 3.

In some embodiments, the compound of Formula II-1 to II-7 can have stereoisomer(s). In such embodiments, the compound of Formula II-1 to II-7 can exist in the form of an individual enantiomer, diastereomer, atropisomer, and/or geometric isomer, as applicable, or a mixture of stereoisomers, including racemic mixtures and mixtures enriched in one or more stereoisomers. In some embodiments, when applicable, the compound of Formula II-1 to II-7 can exist as a mixture of a pair of enantiomers in any ratio, including a racemic mixture with a ratio of 1:1. In some embodiments, when applicable, the compound of Formula II-1 to II-7 can exist as an isolated or enriched individual enantiomer substantially free (e.g., with less than 20%, less than 10%, less than 5%, less than 1%, by weight, by HPLC or SFC area, or both, or with a non-detectable amount) of the other enantiomer.

Typically, in Formula II-1 to II-7 and any of the applicable sub-formulae herein, no more than two (e.g., 0 or 1) of the following pairs can be joined to form a ring as described above: (1) Y and R¹; (2) U and R¹; (3) Z and R³; or (4) R² and R³.

In some embodiments, the present disclosure provides a compound having a structure according to Formula II-1, or a pharmaceutically acceptable salt thereof, with the variables defined and preferred herein. In some embodiments, the present disclosure provides a compound having a structure according to Formula II-2, or a pharmaceutically acceptable salt thereof, with the variables defined and preferred herein. In some embodiments, the present disclosure provides a compound having a structure according to Formula II-3, or a pharmaceutically acceptable salt thereof, with the variables defined and preferred herein. In some embodiments, the present disclosure provides a compound having a structure according to Formula II-4, or a pharmaceutically acceptable salt thereof, with the variables defined and preferred herein. In some embodiments, the present disclosure provides a compound having a structure according to Formula II-5, or a pharmaceutically acceptable salt thereof, with the variables defined and preferred herein. In some embodiments, the present disclosure provides a compound having a structure according to Formula II-6, or a pharmaceutically acceptable salt thereof, with the variables defined and preferred herein. In some embodiments, the present disclosure provides a compound having a structure according to Formula II-7, or a pharmaceutically acceptable salt thereof, with the variables defined and preferred herein.

Unless otherwise specified or contrary from context, the applicable variables R¹, R², R³, U, X, Y, Z, and L² suitable for Formula II-1. II-2, II-3, II-4, II-5, II-6, or II-7 include any of those respective definitions in connection with Formula I (e.g., Formula I-1, I-2, I-3, I-4, I-5, I-6, I-A, I-B, I-C, I-A-1, I-B-1, I-C-1, I-D, I-E, I-L-1, I-L-2, I-L-3, I-L-4, I-L-5, I-L-6, I-L-7, I-L-8, I-L-9, I-L-10, or I-L-11) described and preferred herein in any combinations. For example, in some embodiments, Y is N. In some embodiments, Y is CH. In some embodiments, Y and R¹ together form a 5 or 6 membered heteroaryl having 1-3 ring heteroatoms independently selected from O, S, and N, such as a 5-membered heteroaryl having 1-3 ring nitrogen atoms, preferably,

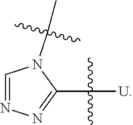

In some embodiments, U is N. In some embodiments, U is CR⁶, wherein R⁶ is defined herein, for example, R⁶ is hydrogen; R⁶ can be C₁₋₄ alkoxy; or R⁶ can be

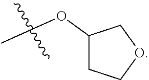 [SMILES: *OC1CCOC1]

In some specific embodiments, R⁶ can be

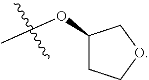 [SMILES: *O[C@@H]1CCOC1]

In some specific embodiments, R⁶ can be

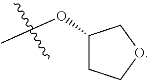 [SMILES: *O[C@H]1CCOC1]

In some embodiments, R¹ is CN. In some embodiments, R¹ can be characterized in that (i) R¹ is halogen, e.g., F or Cl; (ii) R¹ is

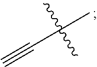 [SMILES: *C#C]

(iii) R¹ is an optionally substituted 5 or 6 membered heteroaryl having 1-4 ring heteroatoms independently selected from O, S, and N, such as an optionally substituted pyrimidinyl or an optionally substituted thiazolyl, for example, R¹ is

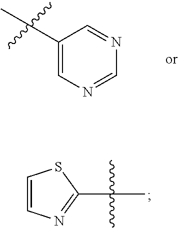 [SMILES: *c1cncnc1 ; *c1nccs1]

or (iv) R¹ is SR^{A1}, wherein R^{A1} is independently optionally substituted C₁₋₄ alkyl, for example, R¹ is SCH₃. In some embodiments, R² can be hydrogen. In some embodiments, R³ is an optionally substituted C₁₋₄ alkyl, such as CH₃, CD₃ etc. In some embodiments, R² and R³ together are joined to form a 5-7 membered heterocycle having 0, 1, or 2 ring heteroatoms selected from O, N, and S, in addition to the nitrogen atom to which R³ is attached, such as

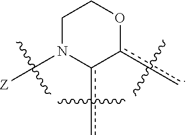

In some embodiments, Z is C(0). In some embodiments, Z is S(O)₂. In some embodiments, Z and R³ together form a 5 or 6 membered heteroaryl having 1-3 ring heteroatoms independently selected from O, S, and N such as a 5-membered heteroaryl having 1-3 (preferably 2 or 3) ring nitrogen atoms, preferably,

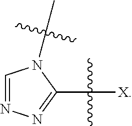

In some embodiments, X is N. In some embodiments, X is CR⁵, wherein R⁵ is CN. In some embodiments, X is CR⁵, wherein R⁵ is CONH₂, COOH, CONH(R^{C1}), CON(R^{C1})₂, or COOR^{C1}, wherein R^{C1} at each occurrence is independently a C₁₋₄ alkyl, such as methyl.

The integer x in Formula II-1 is typically 0.

In some embodiments, the integer m in Formula II-2, II-3, II-4, II-5, II-6, or II-7 can be 0.

In some embodiments, the integer m in Formula II-2, II-3, II-4, II-5, II-6, or II-7 can be 1, and R¹⁴ can be an optionally substituted C₁₋₄ alkyl, such as methyl, ethyl, etc. For example, in some embodiments, the compound of Formula II-3 can be characterized as having a Formula II-3-A, II-3-B, or II-3-C:

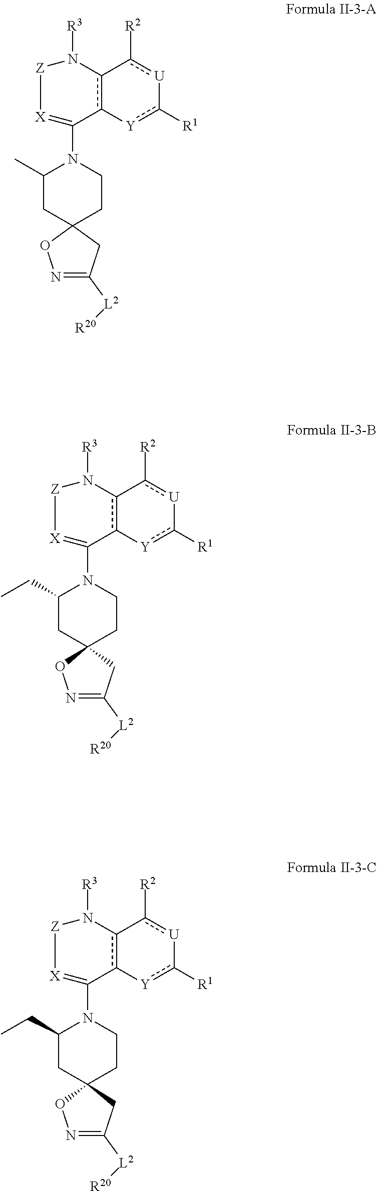

wherein the varies R¹, R², R³, R²⁰, U, X, Y, Z, an L include any of those respective definitions described herein.

Typically, in Formula II-2, II-3, II-4, II-5, II-6, or II-7, L² can be absent.

In some embodiments, L² in Formula II-1 is an optionally substituted C₁₋₄ alkylene selected from:

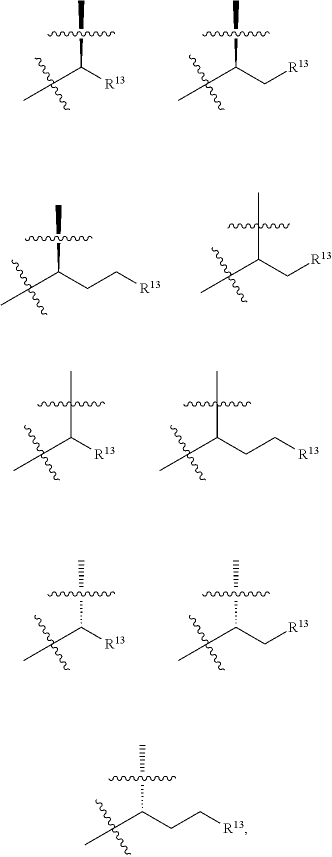

wherein:
R¹³ is selected from hydrogen, halogen (e.g., F, Cl, or Br), CN, OH, NH₂, COOH, CONH₂, SO₂NH₂, COR^{K}, COOR^{K}, CONH(R^{K}), CON(R^{K})₂, SO₂R^{K}, SO₂NH(R^{K}), SO₂N(R^{K})₂, R^{K}, OR^{K}, NH(R^{K}), N(R^{K})₂, SR^{K}, SOR^{K}, SO₂R^{K}, or P(O)(R^{K})₂, wherein R^{K} at each occurrence is independently optionally substituted C₁₋₄ alkyl, optionally substituted C₂₋₄ alkenyl, optionally substituted C₂₋₄ alkynyl, optionally substituted C₁₋₄ heteroalkyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl, wherein, when substituted, the optionally substituted C₁₋₄ alkyl, C₂₋₄ alkenyl, C₂₋₄ alkynyl, C₁₋₄ heteroalkyl, C₃₋₆ cycloalkyl, phenyl, 5 or 6 membered heteroaryl, or 4-7 membered heterocyclyl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. For example, in some embodiments, R¹³ is hydrogen. In some embodiments, R¹³ is CN, OH, COOH, CONH₂, methoxy, ethoxy, cyclopropyl, cyclobutyl, optionally substituted phenyl, or optionally substituted 5 or 6 membered heteroaryl having 1-3 ring heteroatoms independently selected from N, O, and S, wherein, when substituted, the optionally substituted phenyl or 5 or 6 membered heteroaryl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. In some embodiments, R¹³ is a 3-4 membered ring, such as cyclopropyl, cyclobutyl, oxetanyl, azetidinyl, etc., which is optionally substituted, such as with F and/or methyl. For example, in some embodiments, R¹³ is cyclopropyl. In some embodiments, R¹³ is an optionally substituted phenyl or optionally substituted 5 or 6 membered heteroaryl having 1-3 ring heteroatoms independently selected from N, O, and S, such as an optionally substituted phenyl or thiazole, which is optionally substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. For example, in some embodiments, R¹³ is

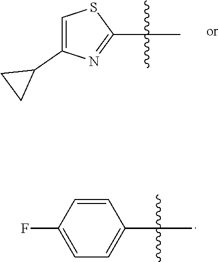 [SMILES: *c1nc(C2CC2)cs1 ; *c1ccc(F)cc1]

In some embodiments, in Formula II-1, II-2, II-3, II-4, II-5, II-6, or II-7, L² can be C(O). In some embodiments, in Formula II-1, I-2, II-3, II-4, II-5, II-6, or II-7, L² can be 0, preferably, in Formula II-1, L² is not 0.

In some embodiments, in Formula II-1, I-2, II-3, II-4, II-5, II-6, or II-7, R²⁰ is optionally substituted phenyl, such as a phenyl which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl. For example, in some embodiments, R²⁰ is a phenyl which is substituted with 1-3 (e.g., 1 or 2) R²², wherein R²² at each occurrence is independently halogen (e.g., F, Cl, or Br), CN, OH, R^{M1}, OR^{M1}, SO₂R^{M1} P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. More preferably, R²⁰ is a phenyl which is substituted with 1-3 (e.g., 1 or 2) R²², wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

In some embodiments, in Formula II-1, I-2, II-3, II-4, II-5, II-6, or II-7, R²⁰ is optionally substituted 5 or 6-membered heteroaryl, such as a pyridyl

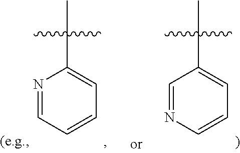 [SMILES: *c1ccccn1 ; *c1cccnc1]

which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl. For example, in some embodiments, R²⁰ is a 5-membered heteroaryl

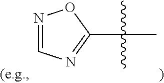 [SMILES: *c1ncno1]

or pyridyl

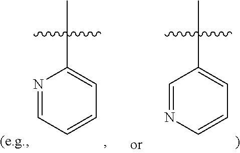 [SMILES: *c1ccccn1 ; *c1cccnc1]

which is substituted with 1-3 (e.g., 1 or 2) R²², as valency permits, wherein R²² at each occurrence is independently halogen (e.g., F, Cl, or Br), CN, OH, R^{M1}, OR^{M1}, SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. More preferably, R²⁰ is a 5-membered heteroaryl

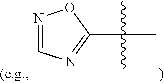 [SMILES: *c1ncno1]

or pyridyl

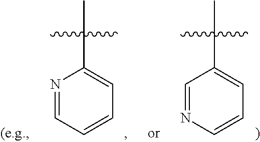 [SMILES: *c1ccccn1 ; *c1cccnc1]

which is substituted with 1-3 (e.g., 1 or 2) R²², as valency permits, wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

In some embodiments, R²⁰ in Formula II-1 to II-7 can have any of the definitions described for Ring A and R⁴ in connection with Formula I and the subformulae.

For example, in some embodiments, R²⁰ can have a structure according to S-1-A, S-1-B, or S-1-C:

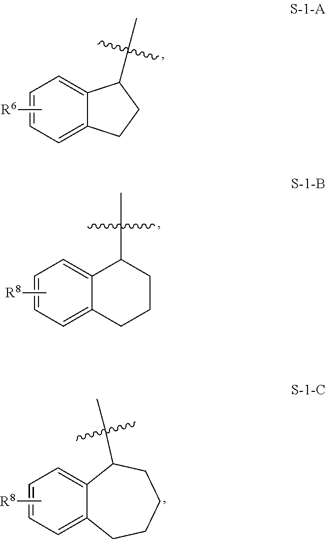

wherein R⁸ is C₁₋₄ alkyl optionally substituted with 1-3 F (such as CH₃, CF₃, etc.), C₁₋₄ alkoxy optionally substituted with 1-3 F (such as OCH₃, OCF₃, etc.), SCF₃, SF₅, cyclopropyl, cyclobutyl, or

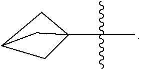 [SMILES: *C12CC(C1)C2]

In some preferred embodiments, R²⁰ in Formula II-1 to II-7 can be selected from the following:

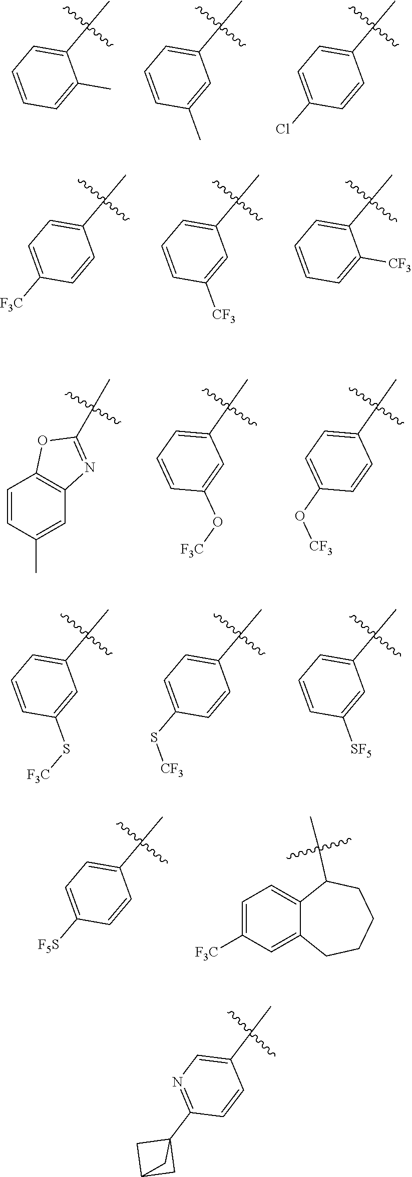

or R²⁰ is selected from:

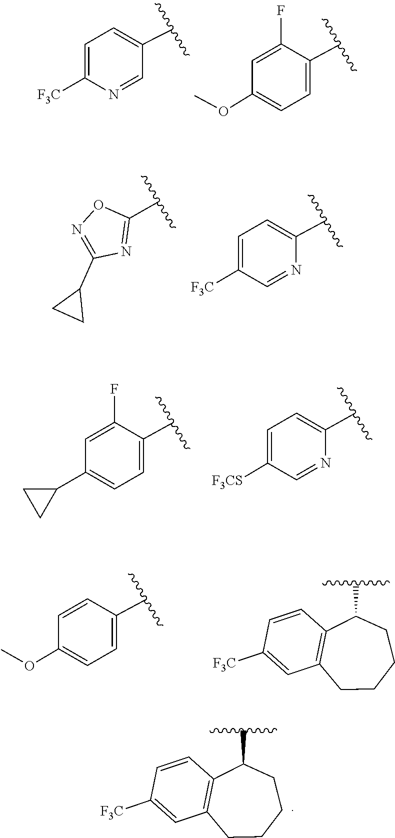

### Formula III

In some embodiments, the present disclosure provides a compound of Formula III-1, III-2, III-3, III-4, III-5, III-6, III-7, III-8, or III-9, or a pharmaceutically acceptable salt thereof:

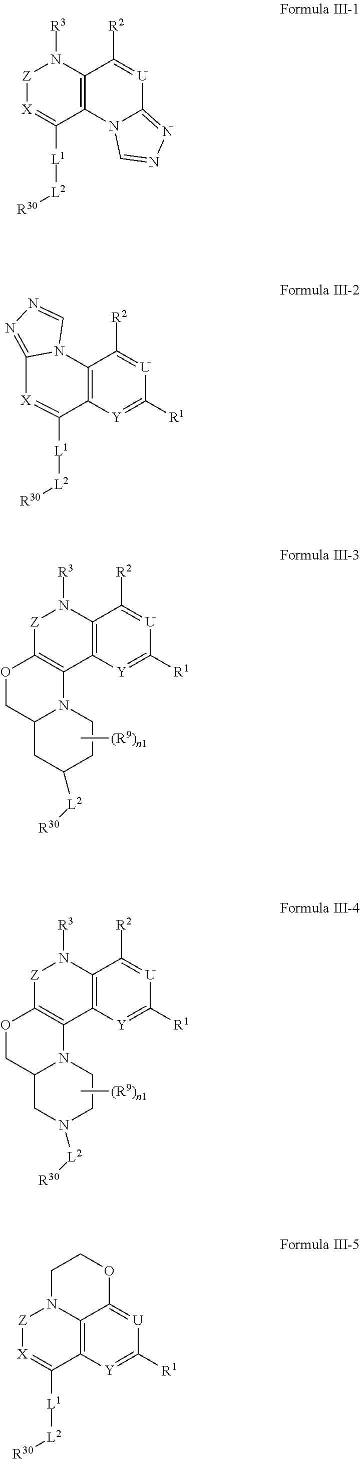

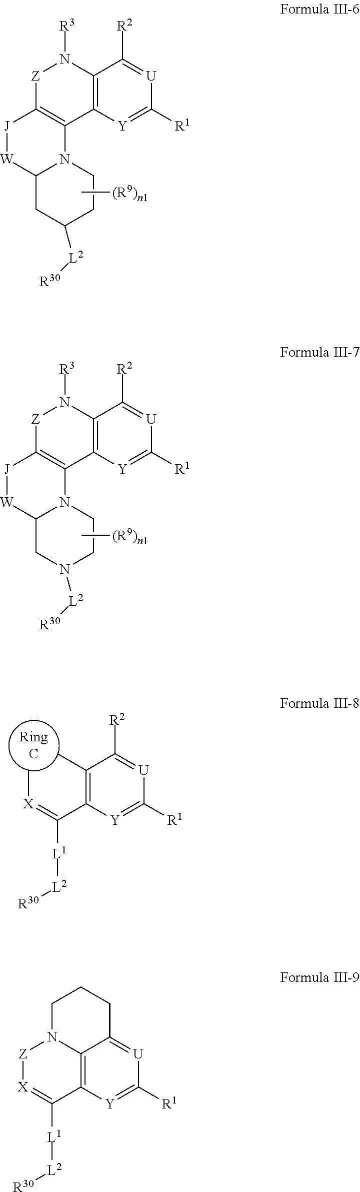

wherein:
   R¹ is hydrogen, halogen (e.g., F, Cl, or Br), CN, OH, NH₂, COOH, CONH₂, R^{A}, OR^{A}, NH(R^{A}), N(R^{A})₂, COOR^{A}, CONH(R^{A}), CON(R^{A})₂, SR^{A}, SOR^{A}, SO₂R^{A}, or P(O)(R^{A})₂, wherein R^{A} at each occurrence is independently optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
   R² is hydrogen, halogen (e.g., F, Cl, or Br), CN, OH, NH₂, R^{B}, OR^{B}, NH(R^{B}), or N(R^{B})₂, wherein R^{B} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, or optionally substituted 4-7 membered heterocyclyl;
   R³ is hydrogen, optionally substituted C₁₋₄ alkyl, optionally substituted C₃₋₆ cycloalkyl, or optionally substituted 4-7 membered heterocyclyl (such as a saturated 4-7 membered heterocyclyl);
   J is O, CO, SO₂, NR¹⁵ or optionally substituted methylene;
   W is absent or -(W¹)ₚ-, wherein p is 1, 2, or 3, wherein W¹ at each occurrence is independently, C(O), NR¹¹, SO₂, O, or optionally substituted methylene, provided that at most one instance of W¹ is C(O), NR¹¹, SO₂, or O; to be clear, when W is absent, the ring containing J is a 5-membered ring; when p is 1, the ring containing J and W is a 6-membered ring; when p is 2 or 3, the ring containing J and W is a 7-membered ring or 8-membered ring, respectively;
   wherein R¹⁵ at each occurrence is independently hydrogen, optionally substituted C₁₋₆ alkyl, optionally substituted C₂₋₆ alkenyl, optionally substituted C₂₋₆ alkynyl, optionally substituted 3-6 membered ring, or nitrogen protecting group;
   X is N or CR⁵, wherein R⁵ is hydrogen, halogen, CN, CONH₂, COOH, CONH(R^{C}), CON(R^{C})₂, OR^{C}, NO₂, or COOR^{C}, wherein R^{C} at each occurrence is independently an optionally substituted C₁₋₄ alkyl;
   U is N or CR⁶, wherein R⁶ is hydrogen, halogen (e.g., F, Cl, or Br), CN, OH, NH₂, R^{D}, OR^{D}, NH(R^{D}), N(R^{D})₂, COOH, CONH₂, COOR^{D}, CONH(R^{D}), CON(R^{D})₂, SR^{D} SOR^{D}, SO₂R^{D}, or P(O)(R^{D})₂, wherein R^{D} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
   Y is N or CR⁷, wherein R⁷ is hydrogen, F, Cl, CN, optionally substituted C₁₋₄ alkyl, or optionally substituted C₁₋₄ heteroalkyl;
   Z is C(O), C(═N—OH), C(═N—O—(C₁₋₄ alkyl)), C(═N—NH₂), C(═N—NH—(C₁₋₄ alkyl)), C(═N—N(C₁₋₄ alkyl)(C₁₋₄ alkyl)), SO, SO₂, S(O)(═NH), or S(O)(═N—(C₁₋₄ alkyl);
   Ring C is a 5 or 6 membered ring, preferably, a 5 or 6 membered heteroaryl having 2-3 ring heteroatoms, such as a 5 membered heteroaryl having 2-3 ring nitrogen atoms, for example, imidazole or triazole ring, wherein Ring C shares two ring atoms with the adjacent ring in Formula III-8;
   L¹ is an optionally substituted 4-12 membered heterocyclylene having one or more rings and 1-4 ring heteroatoms each independently O, N, or S, or —N(R^{E})—, wherein R^{E} is optionally substituted C₁₋₄ alkyl or optionally substituted 3-7 membered ring;
   L² is absent, O, NH, —N(R^{E2})—, C(O), SO₂, an optionally substituted C₁₋₄ alkylene, an optionally substituted C₂₋₄ alkenylene, an optionally substituted C₁₋₄ alkynylene, optionally substituted C₁₋₄ heteroalkylene, or an optionally substituted 3-7 membered ring, wherein R^{E2} is optionally substituted C₁₋₄ alkyl or optionally substituted 3-7 membered ring;
   R³⁰ is hydrogen, an optionally substituted phenyl, optionally substituted 5 or 6 membered heteroaryl, or optionally substituted 8-12 membered ring having two or more rings;
   R⁹ at each occurrence is independently R^{N}, COOR^{N}, COR^{N}, CONH(R^{N}), or CON(R^{N})₂, wherein R^{N} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, or optionally substituted 5 or 6 membered heteroaryl; and
   n1 is 0, 1, 2, or 3.

In some embodiments, the compound of Formula III-1 to III-9 can have stereoisomer(s). In such embodiments, the compound of Formula III-1 to III-9 can exist in the form of an individual enantiomer, diastereomer, atropisomer, and/or geometric isomer, as applicable, or a mixture of stereoisomers, including racemic mixtures and mixtures enriched in one or more stereoisomers. In some embodiments, when applicable, the compound of Formula III-1 to III-9 can exist as a mixture of a pair of enantiomers in any ratio, including a racemic mixture with a ratio of 1:1. In some embodiments, when applicable, the compound of Formula III-1 to III-9 can exist as an isolated or enriched individual enantiomer substantially free (e.g., with less than 20%, less than 10%, less than 5%, less than 1%, by weight, by HPLC or SFC area, or both, or with a non-detectable amount) of the other enantiomer.

In some embodiments, the present disclosure provides a compound having a structure according to Formula III-1, or a pharmaceutically acceptable salt thereof, with the variables defined and preferred herein. In some embodiments, the present disclosure provides a compound having a structure according to Formula III-2, or a pharmaceutically acceptable salt thereof, with the variables defined and preferred herein. In some embodiments, the present disclosure provides a compound having a structure according to Formula III-3, or a pharmaceutically acceptable salt thereof, with the variables defined and preferred herein. In some embodiments, the present disclosure provides a compound having a structure according to Formula III-4, or a pharmaceutically acceptable salt thereof, with the variables defined and preferred herein. In some embodiments, the present disclosure provides a compound having a structure according to Formula III-5, or a pharmaceutically acceptable salt thereof, with the variables defined and preferred herein. In some embodiments, the present disclosure provides a compound having a structure according to Formula III-6, or a pharmaceutically acceptable salt thereof, with the variables defined and preferred herein. In some embodiments, the present disclosure provides a compound having a structure according to Formula III-7, or a pharmaceutically acceptable salt thereof, with the variables defined and preferred herein. In some embodiments, the present disclosure provides a compound having a structure according to Formula III-8, or a pharmaceutically acceptable salt thereof, with the variables defined and preferred herein. In some embodiments, the compound of Formula III-8 can be characterized as having a structure according to Formula III-8-A:

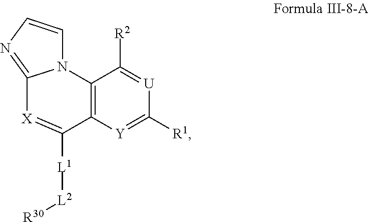

wherein the variables R¹, R², U, X, Y, L¹, L², and R³⁰ are defined herein, such as those described herein below in connection with Formula III-2 in enumerated embodiments A2-A25. In some embodiments, the present disclosure provides a compound having a structure according to Formula III-9, or a pharmaceutically acceptable salt thereof, with the variables defined and preferred herein.

Unless otherwise specified or contrary from context, the applicable variables R¹, R², R³, U, X, Y, Z, L¹, L², n1, and R⁹ suitable for Formula III-1, III-2, III-3, III-4, III-5, III-6, III-7, III-8, or III-9, include any of those respective definitions in connection with Formula I (e.g., Formula I-1, I-2, I-3, I-4, I-5, I-6, I-A, I-B, I-C, I-A-1, I-B-1, I-C-1, I-D, I-E, I-L-1, I-L-2, I-L-3, I-L-4, I-L-5, I-L-6, I-L-7, I-L-8, I-L-9, I-L-10, or I-L-11), Formula II-1, II-2, II-3, II-4, II-5, II-6, or II-7 described and preferred herein in any combinations. For example, in some embodiments, Y is N. In some embodiments, Y is CH. In some embodiments, U is N. In some embodiments, U is CR⁶, wherein R⁶ is defined herein, for example, R⁶ is hydrogen; R⁶ can be C₁₋₄ alkoxy; or R⁶ can be

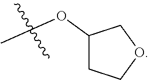 [SMILES: *OC1CCOC1]

In some specific embodiments, R⁶ can be

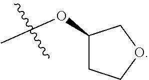 [SMILES: *O[C@@H]1CCOC1]

In some specific embodiments, R⁶ can be

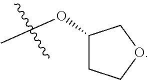 [SMILES: *O[C@H]1CCOC1]

In some embodiments, R¹ is CN. In some embodiments, R¹ can be characterized in that (i) R¹ is halogen, e.g., F or Cl; (ii) R¹ is

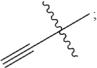 [SMILES: *C#C]

(iii) R¹ is an optionally substituted 5 or 6 membered heteroaryl having 1-4 ring heteroatoms independently selected from O, S, and N, such as an optionally substituted pyrimidinyl or an optionally substituted thiazolyl, for example, R¹ is N or

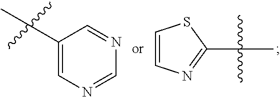

or (iv) R¹ is SR^{A1}, wherein R^{A1} is independently optionally substituted C₁₋₄ alkyl, for example, R¹ is SCH₃. In some embodiments, R² can be hydrogen. In some embodiments, R³ is an optionally substituted C₁₋₄ alkyl, such as CH₃, CD₃ etc. In some embodiments, Z is C(0). In some embodiments, Z is S(O)₂. In some embodiments, X is N. In some embodiments, X is CR⁵, wherein R⁵ is CN. In some embodiments, X is CR⁵, wherein R⁵ is CONH₂, COOH, CONH(R^{C1}), CON(R^{C1})₂, or COOR^{C1}, wherein Rei at each occurrence is independently a C₁₋₄ alkyl, such as methyl. In some embodiments, n1 is 0. In some embodiments, n1 is 1. In some embodiments, R⁹ at each occurrence is independently CH₃, CH₂CH₃, CH₂CH₂CH₃, fluorine-substituted C₁₋₃ alkyl (e.g., CF₂H), cyclopropyl, or cyclobutyl, CH₂OH, CH₂OCH₃, CH₂OCH₂CH₃, CH₂NH₂, CH₂N₃, or CH₂NHC(O)OCH₃.

In some embodiments, in Formula III-6 or III-7, J is NR¹, wherein R¹⁵ is defined herein. For example, in some embodiments, J is N(C₁₋₄ alkyl), such as NCH₃.

In some embodiments, in Formula III-6 or III-7, J is an optionally substituted methylene. For example, in some embodiments, J is CH₂. In some embodiments, J is CF₂, CHCH₃, C(CH₃)₂, CHOH, etc.

In some embodiments, in Formula III-6 or III-7, J is O.

In some embodiments, in Formula III-6 or III-7, W is absent. Thus, the ring containing J is a 5 membered ring.

In some embodiments, in Formula III-6 or III-7, p is 1 and W is W¹, wherein W¹ is defined herein. For example, in some embodiments, W¹ is C(O), SO₂, CH₂, CF₂, CHCH₃, C(CH₃)₂, CHOH, etc. In such embodiments, the ring containing J and W is a 6-membered ring.

In some embodiments, in Formula III-6 or III-7, p is 2 and W is —W¹—W¹—, wherein each W¹ is defined herein. For example, in some embodiments, each W¹ is independently C(O), SO₂, CH₂, CF₂, CHCH₃, C(CH₃)₂, CHOH, etc. In some embodiments, both W¹ can be CH₂. In such embodiments, the ring containing J and W is a 7-membered ring.

Typically, in Formula III-1, III-2, III-5, III-8, or III-9, L¹ is an optionally substituted 4-12 membered heterocyclylene herein.

In some embodiments, in Formula III-1, III-2, III-5, III-8, or III-9, L¹ can be

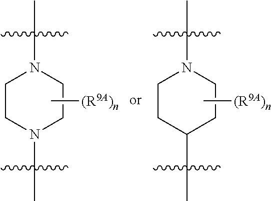

(either of the two attaching points of the piperidine, N or C, can be attached to L²),
wherein:
n is 0, 1, 2, 3, or 4, and
   (1) R^{9A} at each occurrence is independently R^{N}, COOR^{N}, COR^{N}, CONH(R^{N}), or CON(R^{N})₂, wherein R^{N} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, or optionally substituted 5 or 6 membered heteroaryl; or
   (2) two instances of R^{9A} can be joined to form a double bond or a 3-5 membered ring and any remaining instance(s) of R^{9A} are defined as in (1).
      For example, in some embodiments, R^{9A} at each occurrence is independently CH₃, CH₂CH₃, CH₂CH₂CH₃, fluorine-substituted C₁₋₃ alkyl (e.g., CF₂H), cyclopropyl, or cyclobutyl, CH₂OH, CH₂OCH₃, CH₂OCH₂CH₃, CH₂NH₂, CH₂N₃, or CH₂NHC(O)OCH₃. Typically, in Formula III-1. III-2, III-5, III-8, or III-9, n is 0, 1, or 2. For example, L¹ can be selected from

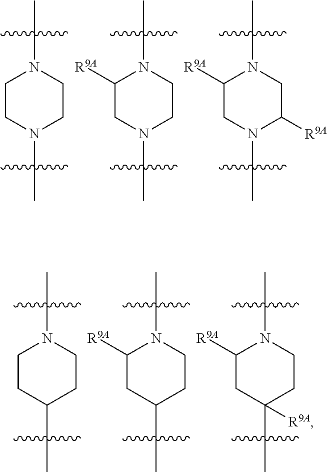

wherein either of the two attaching points can be attached to L².

In some preferred embodiments, in Formula III-1, III-2, III-5, III-8, or III-9, L¹ can be selected from the following (the bottom attaching point, N or C, is attached to L²):

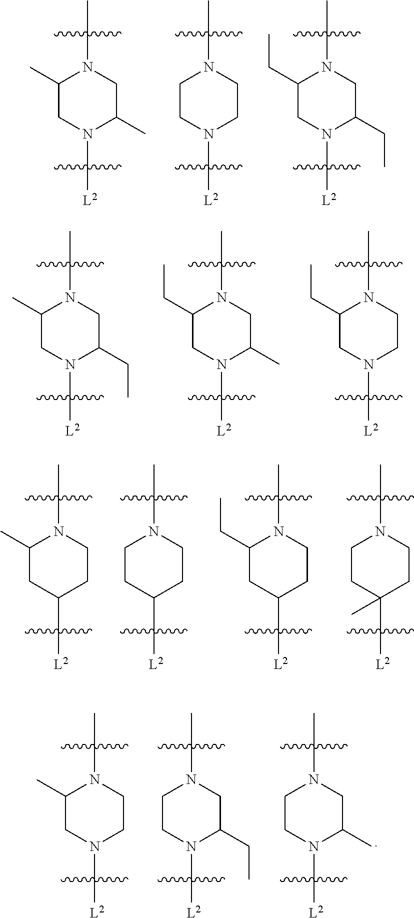

In some preferred embodiments, in Formula III-1, III-2, III-5, III-8, or III-9, L¹ can be selected from the following (the bottom attaching point, N or C, is attached to L²):

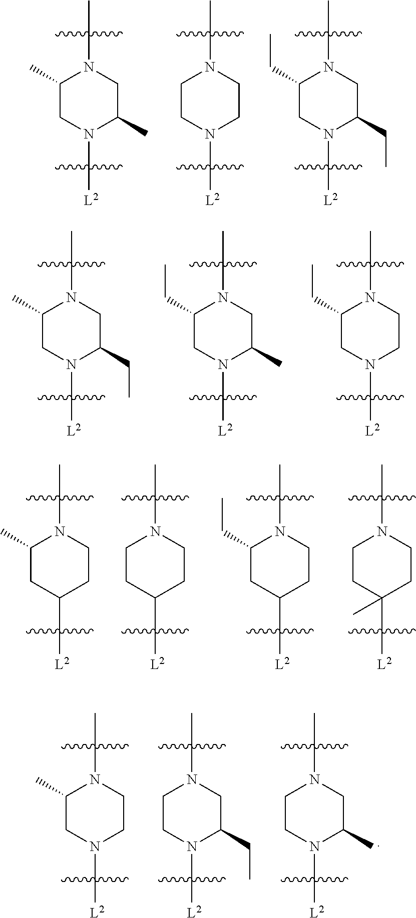

In some embodiments, L¹ in Formula III-1, III-2, III-5, III-8, or III-9 can also be an optionally substituted 7-12 membered heterocyclylene having two or more rings and 1-4 ring heteroatoms each independently O, N, or S, when substituted, the substituent(s) can be attached to any one or more of the two or more rings. For example, in some embodiments, L¹ can be an optionally substituted 8-12 membered fused, spiro, or bridged bicyclic heterocyclylene having 1-4 ring heteroatoms each independent O, N, or S, wherein each ring of the bicyclic heterocyclylene can be saturated, partially unsaturated, or aromatic, and each ring of the bicyclic heterocyclylene can have 0, 1, 2, or 3 ring heteroatoms, provided that the bicyclic heterocyclylene as a whole is not fully aromatic and the total number of heteroatoms in the bicyclic heterocyclylene does not exceed 4. In some embodiments, L¹ can be an optionally substituted 8-11 membered fused bicyclic heterocyclylene having 1-3 ring heteroatoms each independent O, N, or S, wherein (1) one of the two fused rings is phenyl or 5 or 6 membered heteroaryl, and (2) the other of the two fused rings is a 5-7 membered heterocycle having one or two ring heteroatoms each independently O, N, or S, preferably, the 5-7 membered heterocycle has at least one ring nitrogen atom. In some embodiments, L¹ can be an optionally substituted 8-11 (e.g., 8, 9, or 10) membered spiro bicyclic heterocyclylene having 1-4 ring heteroatoms each independent O, N, or S, wherein (1) one of the two spiro rings is a 5-7 membered heterocycle having one or two ring heteroatoms each independently O, N, or S, preferably, the 5-7 membered heterocycle has one ring nitrogen atom, and (2) the other of the two spiro rings is a 4-6 membered heterocycle having 1-3 ring heteroatoms each independently O, N, or S, for example, one of the two spiro rings is a pyrrolidine, piperidine, azepane ring and the other of the two spiro rings is azetidine, pyrrolidine, pyrrolidinone, piperidinone, oxazoline, isoxazoline, thiazoline, isothiazoline, etc.

For example, in some preferred embodiments, L¹ in Formula III-1, III-2, III-5, III-8, or III-9 can be selected from the following (the bottom attaching point, N or C, is attached to L²):

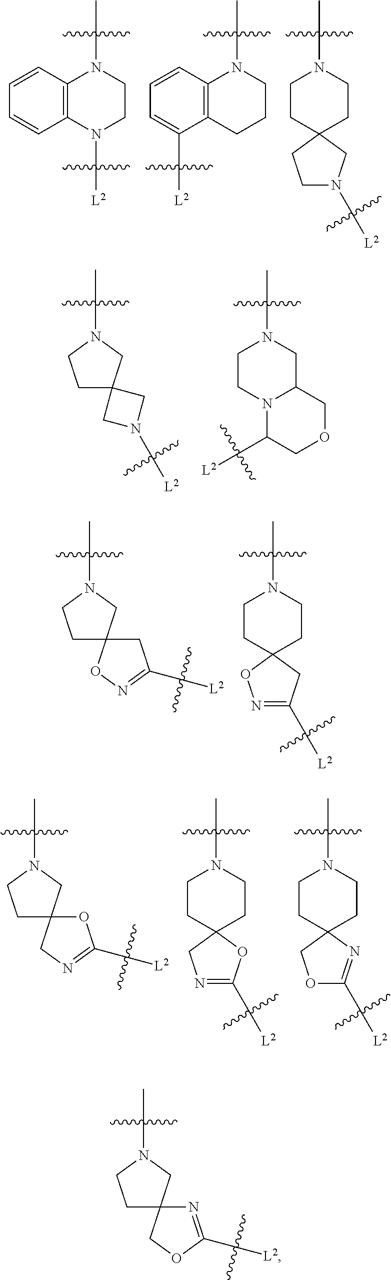

wherein in each of the bicyclic heterocyclylene, each of the two rings can be optionally substituted with 1-3 R¹⁰, wherein R¹⁰ at each occurrence is independently halogen, OH, NH₂, oxo (as applicable), R^{J}, OR^{J}, CN, NH(R^{J}), or N(R^{J})₂, wherein R^{J} at each occurrence is independently an optionally substituted C₁₋₄ alkyl, optionally substituted C₂₋₄ alkenyl, optionally substituted C₂₋₄ alkynyl, or optionally substituted 3-4 membered ring.

In some preferred embodiments, L¹ in Formula III-1, III-2, III-5, III-8, or III-9 can be selected from the following (the bottom attaching point, N or C, is attached to L²):

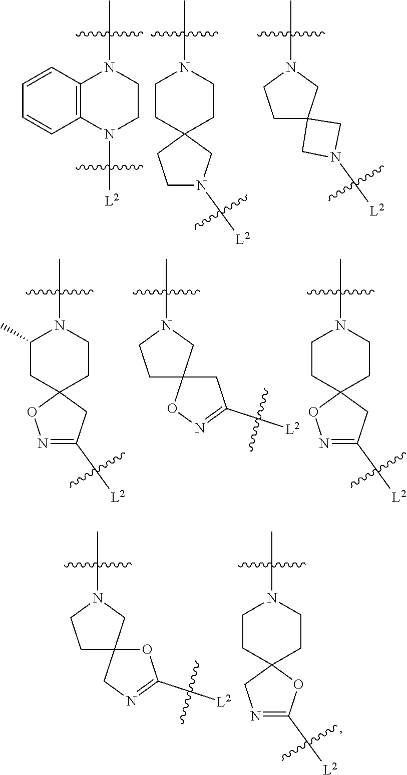

or L¹ is selected from

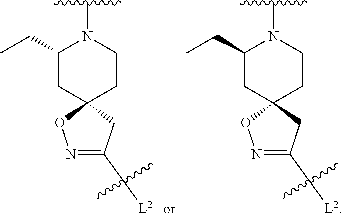

In some embodiments, L¹ in Formula III-1, III-2, III-5, III-8, or III-9 can also be —N(C₁₋₄ alkyl)-, such as —N(CH₃)—.

In some embodiments, in Formula III-1, III-2, III-3, III-4, III-5, III-6, III-7, III-8, or III-9, L² can be absent.

In some embodiments, L² in Formula III-1, III-2, III-3, III-4, III-5, III-6, III-7, III-8, or III-9 can be an optionally substituted C₁₋₄ alkylene selected from:

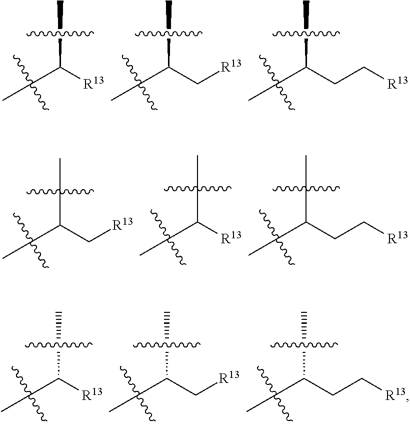

wherein:
R¹³ is selected from hydrogen, halogen (e.g., F, Cl, or Br), CN, OH, NH₂, COOH, CONH₂, SO₂NH₂, COR^{K}, COOR^{K}, CONH(R^{K}), CON(R^{K})₂, SO₂R^{K}, SO₂NH(R^{K}), SO₂N(R^{K})₂, R^{K}, OR^{K}, NH(R^{K}), N(R^{K})₂, SR^{K}, SOR^{K}, SO₂R^{K}, or P(O)(R^{K})₂, wherein R^{K} at each occurrence is independently optionally substituted C₁₋₄ alkyl, optionally substituted C₂₋₄ alkenyl, optionally substituted C₂₋₄ alkynyl, optionally substituted C₁₋₄ heteroalkyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl, wherein, when substituted, the optionally substituted C₁₋₄ alkyl, C₂₋₄ alkenyl, C₂₋₄ alkynyl, C₁₋₄ heteroalkyl, C₃₋₆ cycloalkyl, phenyl, 5 or 6 membered heteroaryl, or 4-7 membered heterocyclyl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. For example, in some embodiments, R¹³ is hydrogen. In some embodiments, R¹³ is CN, OH, COOH, CONH₂, methoxy, ethoxy, cyclopropyl, cyclobutyl, optionally substituted phenyl, or optionally substituted 5 or 6 membered heteroaryl having 1-3 ring heteroatoms independently selected from N, O, and S, wherein, when substituted, the optionally substituted phenyl or 5 or 6 membered heteroaryl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. In some embodiments, R¹³ is a 3-4 membered ring, such as cyclopropyl, cyclobutyl, oxetanyl, azetidinyl, etc., which is optionally substituted, such as with F and/or methyl. For example, in some embodiments, R¹³ is cyclopropyl. In some embodiments, R¹³ is an optionally substituted phenyl or optionally substituted 5 or 6 membered heteroaryl having 1-3 ring heteroatoms independently selected from N, O, and S, such as an optionally substituted phenyl or thiazole, which is optionally substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. For example, in some embodiments, R¹³ is

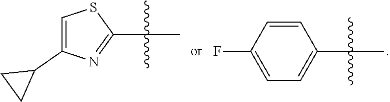 [SMILES: *c1nc(C2CC2)cs1 ; *c1ccc(F)cc1]

In some embodiments, in Formula III-1, III-2, III-3, III-4, III-5, III-6, III-7, III-8, or III-9, L² can be C(O). In some embodiments, in Formula III-1, III-2, III-3, III-4, III-5, III-6, III-7, III-8, or III-9, L² can be O, preferably, in Formula III-4, L² is not O.

In some embodiments, in Formula III-1, III-2, III-3, III-4, III-5, III-6, III-7, III-8, or III-9, L² can be an optionally substituted 5 or 6 membered heteroarylene, for example,

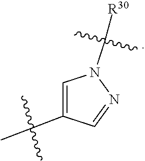

In some embodiments, in Formula III-1, III-2, III-5, III-8, or III-9, L¹ can be —N(R^{E})—, L² is an optionally substituted phenylene, e.g.,

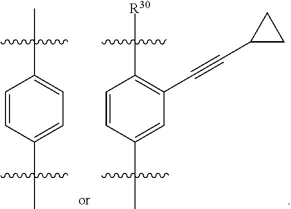

and R³⁰ is hydrogen. For example, in some embodiments, L¹, L², and R³⁰ together is:

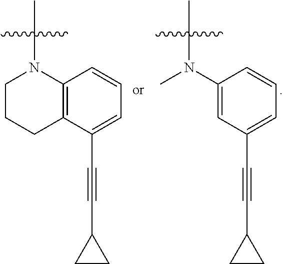

In some embodiments, in Formula III-1, III-2, III-3, III-4, III-5, III-6, III-7, III-8, or III-9, R³⁰ is optionally substituted phenyl, such as a phenyl which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from 0, S, and N, or optionally substituted 4-7 membered heterocyclyl. For example, in some embodiments, R³⁰ is a phenyl which is substituted with 1-3 (e.g., 1 or 2) R²², wherein R²² at each occurrence is independently halogen (e.g., F, Cl, or Br), CN, OH, R^{M1}, OR^{M1} SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. More preferably, R³⁰ is a phenyl which is substituted with 1-3 (e.g., 1 or 2) R²², wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

In some embodiments, in Formula III-1, III-2, III-3, III-4, III-5, III-6, III-7, III-8, or III-9, R³⁰ is optionally substituted 5 or 6-membered heteroaryl, such as a pyridyl

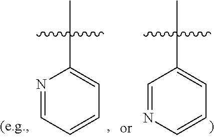 [SMILES: *c1ccccn1 ; *c1cccnc1]

which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl. For example, in some embodiments, R³⁰ is a 5-membered heteroaryl

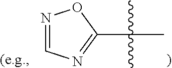 [SMILES: *c1ncno1]

or pyridyl

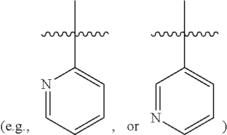 [SMILES: *c1ccccn1 ; *c1cccnc1]

which is substituted with 1-3 (e.g., 1 or 2) R²², as valency permits, wherein R²² at each occurrence is independently halogen (e.g., F, Cl, or Br), CN, OH, R^{M1}, OR^{M1}, SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. More preferably, R³⁰ is a 5-membered heteroaryl

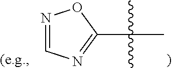 [SMILES: *c1ncno1]

or pyridyl

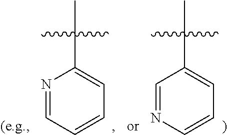 [SMILES: *c1ccccn1 ; *c1cccnc1]

Which is substituted with 1-3 (e.g., 1 or 2) R²², as valency permits, wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

In some embodiments, R³⁰ in Formula III-1 to III-9 can have any of the definitions described for Ring A and R⁴ in connection with Formula I and the subformulae.

For example, in some embodiments, R³⁰ can have a structure according to S-1-A, S-1-B, or S-1-C:

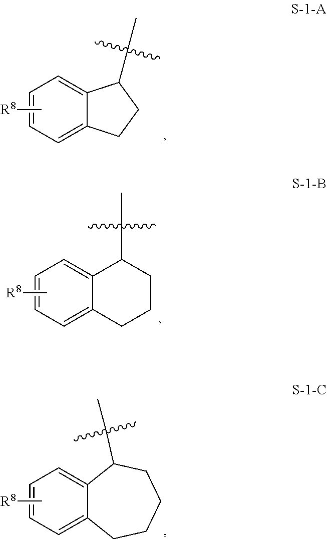

wherein R⁸ is C₁₋₄ alkyl optionally substituted with 1-3 F (such as CH₃, CF₃, etc.), C₁₋₄ alkoxy optionally substituted with 1-3 F (such as OCH₃, OCF₃, etc.), SCF₃, SF₅, cyclopropyl, cyclobutyl, or

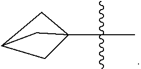 [SMILES: *C12CC(C1)C2]

In some preferred embodiments, R³⁰ in Formula III-1 to III-9 can be selected from the following:

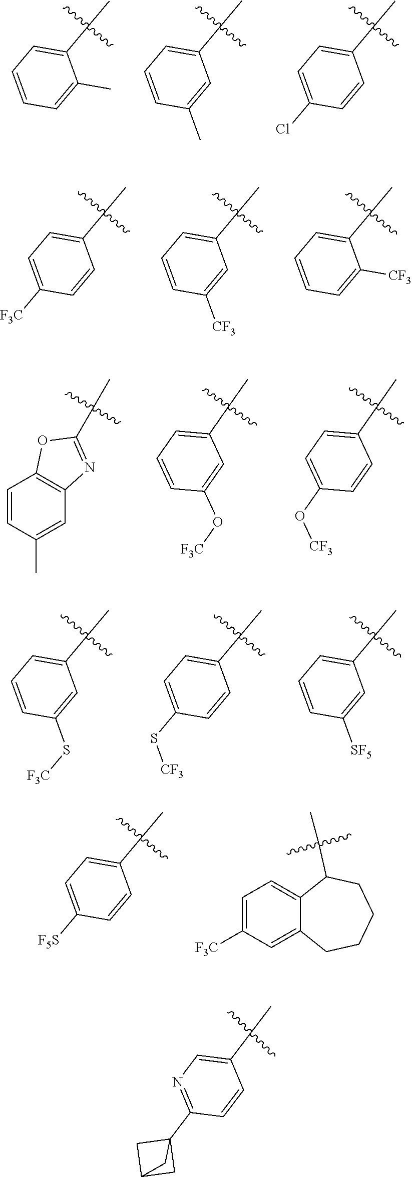

or R³⁰ is selected from:

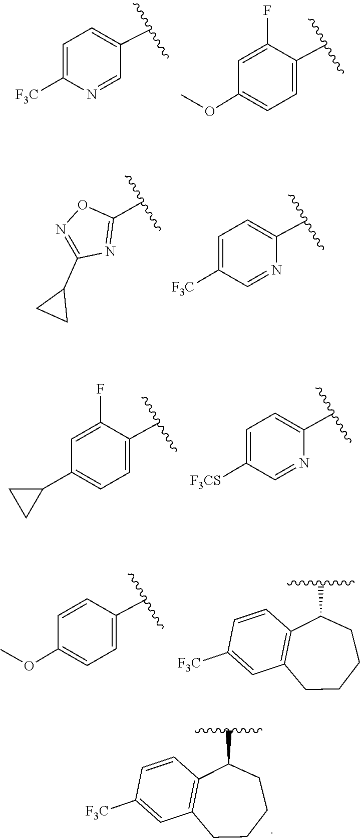

In some embodiments, in Formula III-1, III-2, III-5, III-8, or III-9, L¹, L² and R³⁰ (i.e., L¹-L²-R³⁰) together can be selected from:

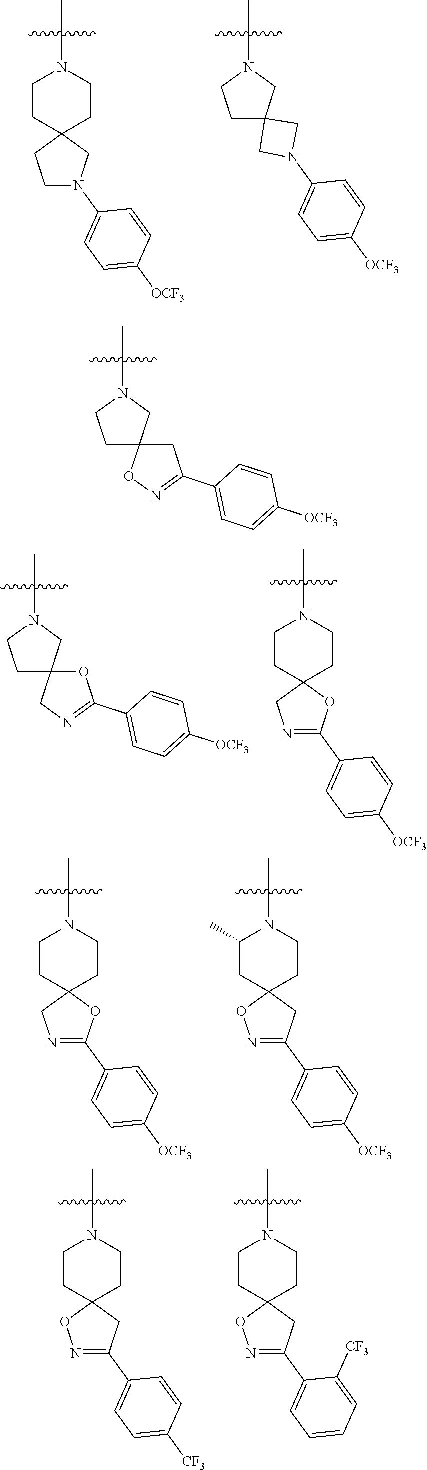

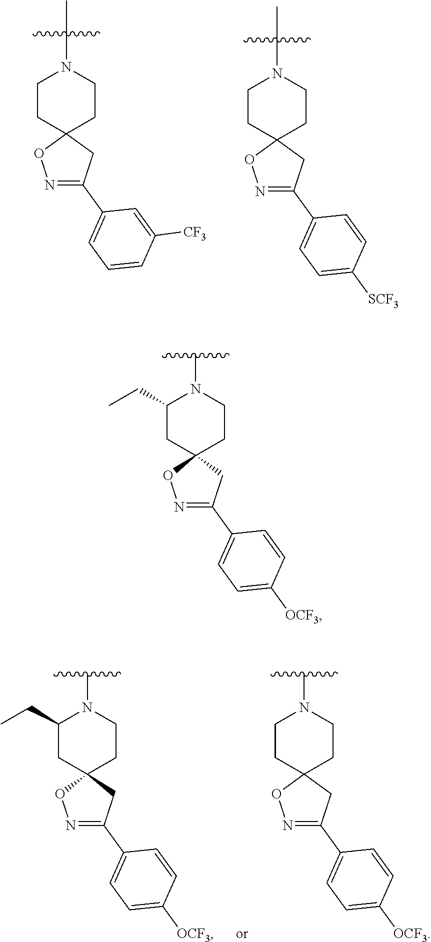

In some embodiments, in Formula III-1, III-2, III-5, III-8, or III-9, L¹, L², and R³⁰ together can be selected from:

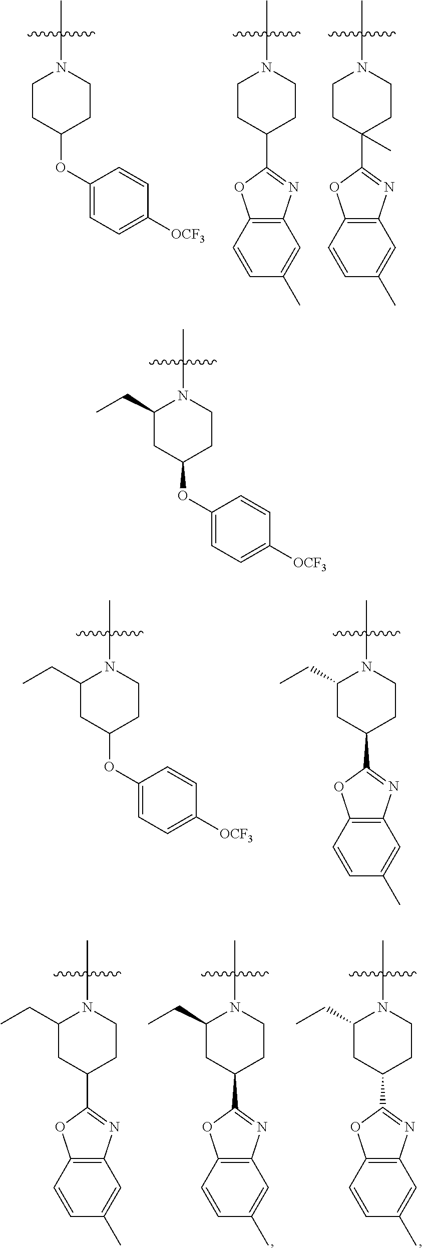

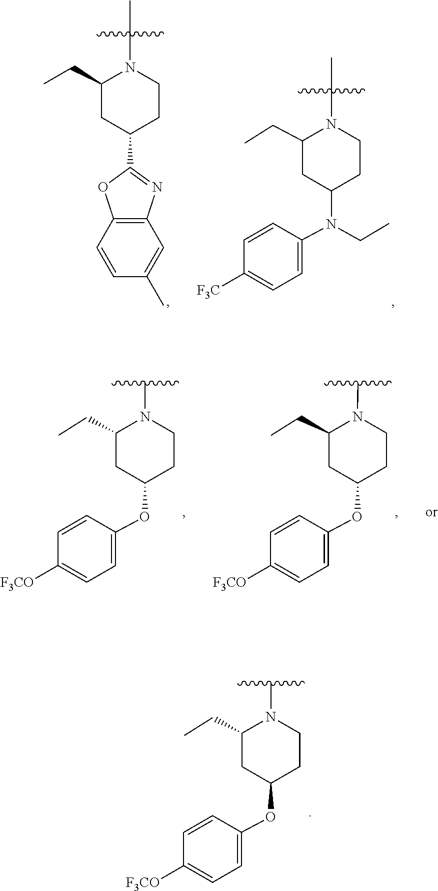 [SMILES: *N1CC[C@H](c2nc3cc(C)ccc3o2)C[C@H]1CC ; *N1CCC(N(CC)c2ccc(C(F)(F)F)cc2)CC1CC ; *N1CC[C@H](Oc2ccc(OC(F)(F)F)cc2)C[C@@H]1CC ; *N1CC[C@H](Oc2ccc(OC(F)(F)F)cc2)C[C@H]1CC ; *N1CC[C@@H](Oc2ccc(OC(F)(F)F)cc2)C[C@@H]1CC]

In some embodiments, in Formula III-1, III-2, III-5, III-8, or III-9, L¹, L², and R³⁰ together can be selected from:

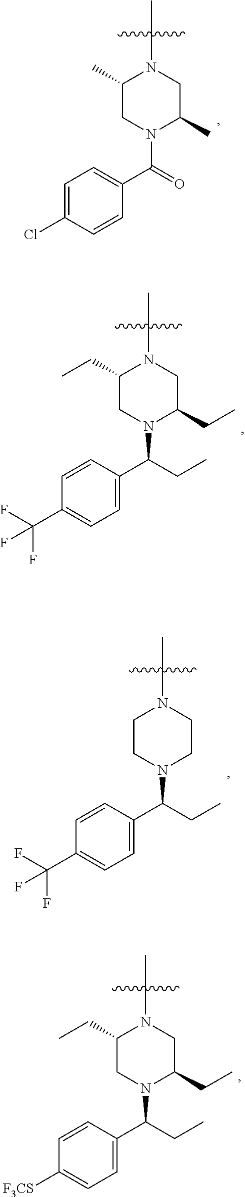

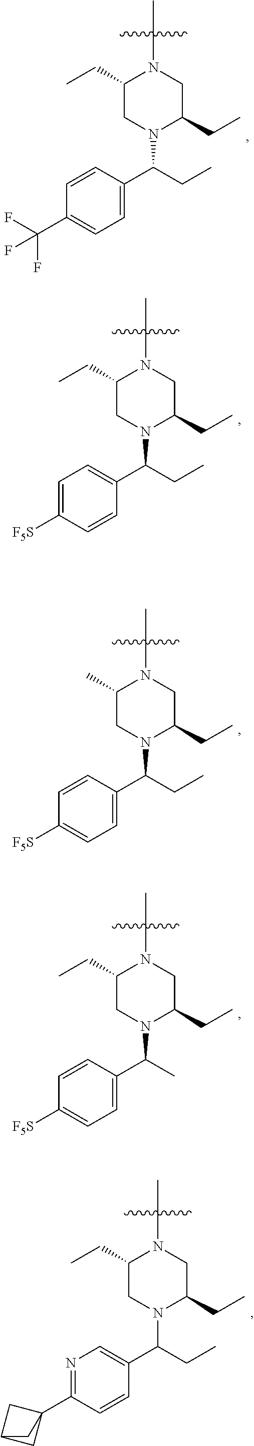

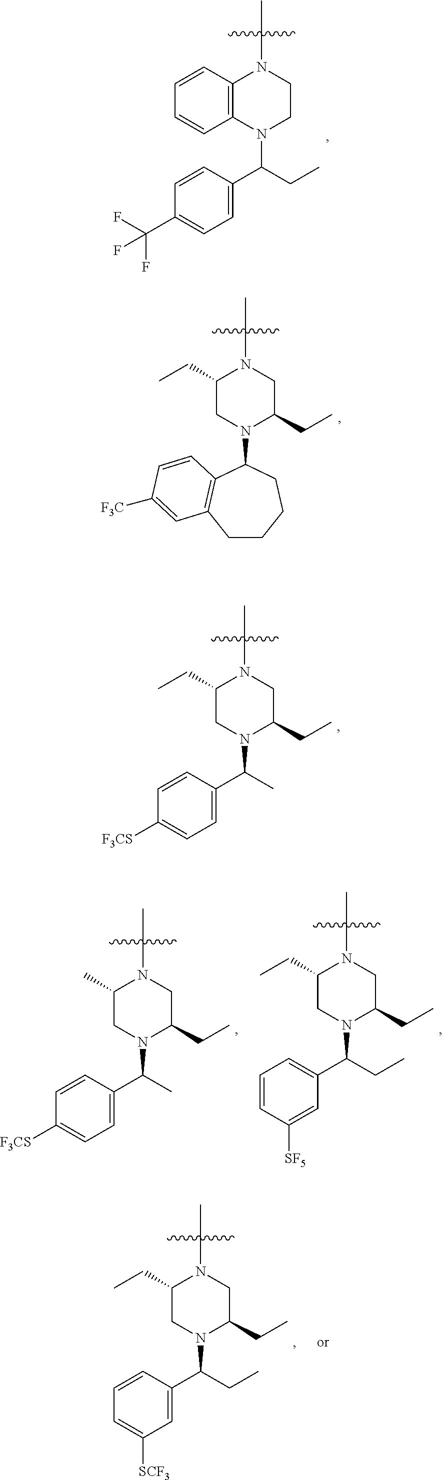

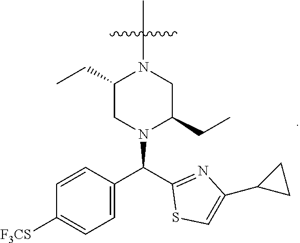

In some embodiments, in Formula III-1, III-2, III-5, III-8, or III-9, L¹-L²-R³⁰ is selected from the following:

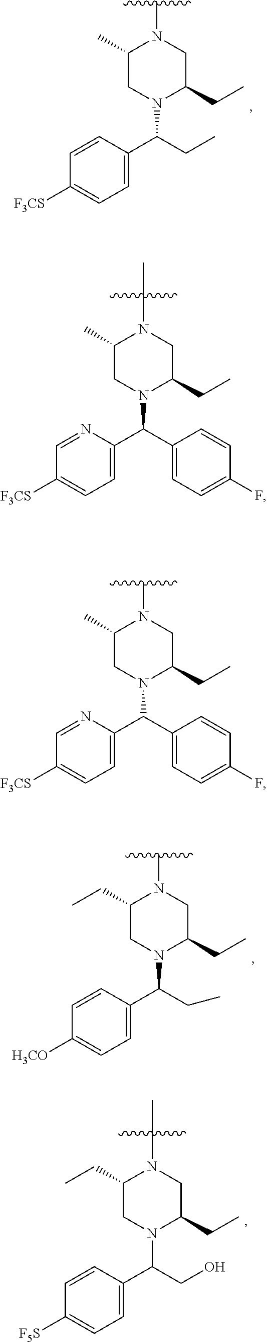

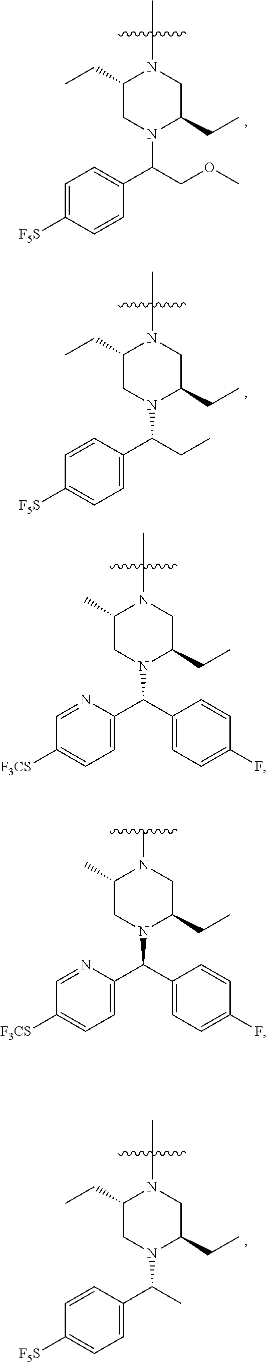

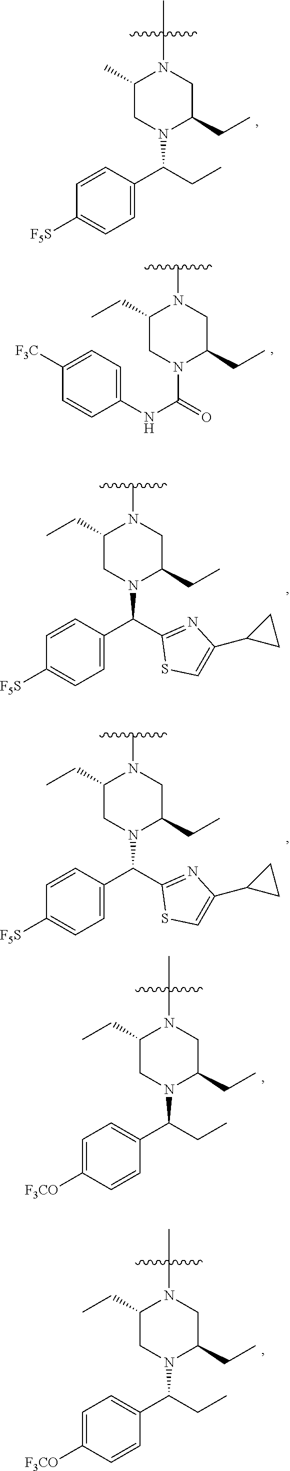

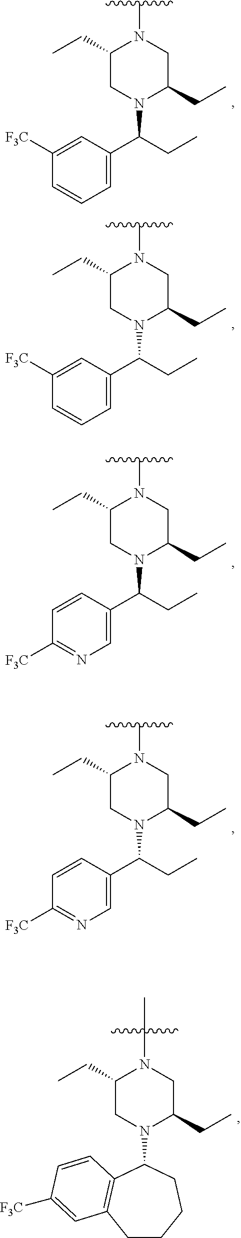 [SMILES: *N1C[C@@H](CC)N([C@@H](CC)c2cccc(C(F)(F)F)c2)C[C@@H]1CC ; *N1C[C@@H](CC)N([C@H](CC)c2cccc(C(F)(F)F)c2)C[C@@H]1CC ; *N1C[C@@H](CC)N([C@@H](CC)c2ccc(C(F)(F)F)nc2)C[C@@H]1CC ; *N1C[C@@H](CC)N([C@H](CC)c2ccc(C(F)(F)F)nc2)C[C@@H]1CC ; *N1C[C@@H](CC)N([C@@H]2CCCCc3cc(C(F)(F)F)ccc32)C[C@@H]1CC]

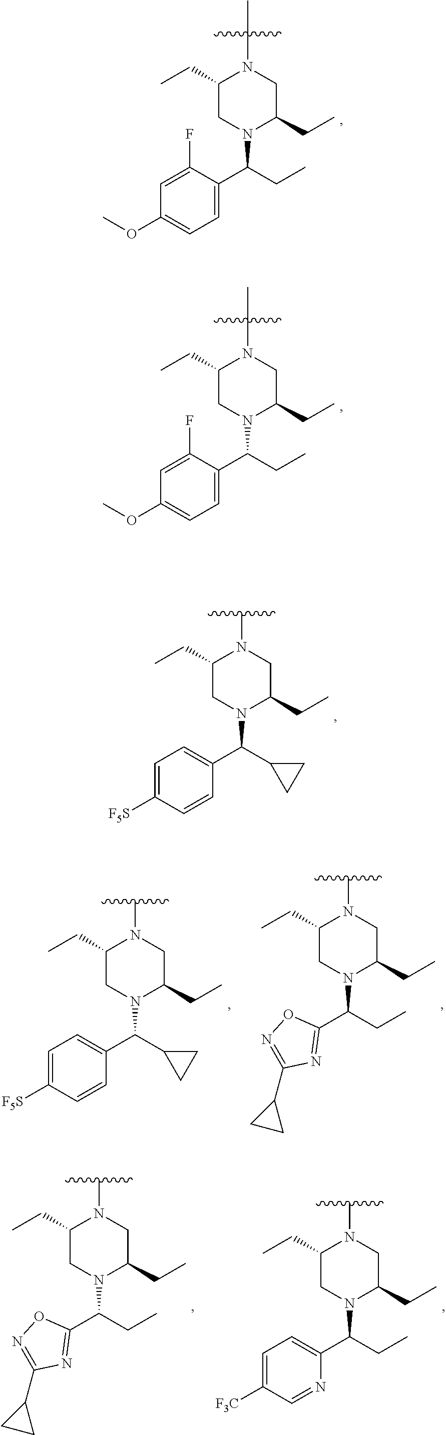

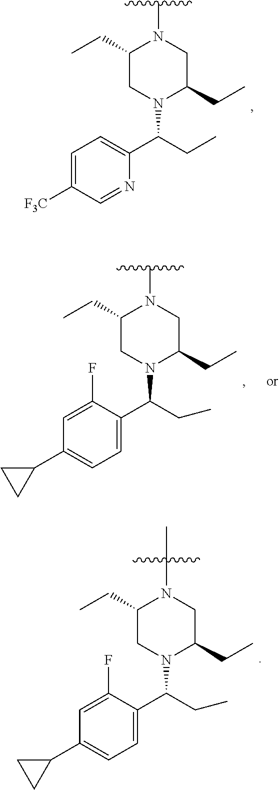 [SMILES: *N1C[C@@H](CC)N([C@H](CC)c2ccc(C(F)(F)F)cn2)C[C@@H]1CC ; *N1C[C@@H](CC)N([C@@H](CC)c2ccc(C3CC3)cc2F)C[C@@H]1CC ; *N1C[C@@H](CC)N([C@H](CC)c2ccc(C3CC3)cc2F)C[C@@H]1CC]

In some embodiments, in Formula III-1, III-2, III-5, III-8, or III-9, L¹, L², and R³⁰ together can be selected from:

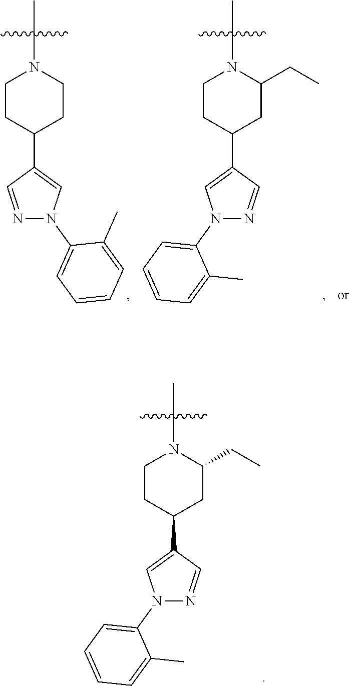

In some embodiments, the present disclosure provides the following enumerated exemplified Embodiments A1-A26:
Embodiment A1. A compound of Formula III-2, or a pharmaceutically acceptable salt thereof,

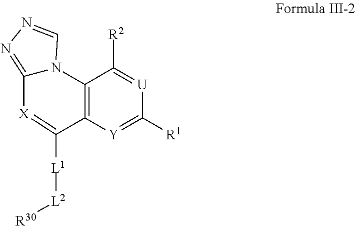

wherein the variables X, Y, U, R¹, R², L¹, L², and R³⁰ are defined herein.
Embodiment A2. The compound of Embodiment A1, or a pharmaceutically acceptable salt thereof, characterized as having a structure according to Formula III-2-A:

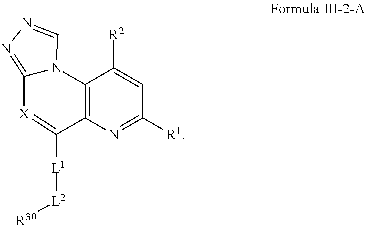

Embodiment A3. The compound of Embodiment A1, or a pharmaceutically acceptable salt thereof, characterized as having a structure according to Formula III-2-B:

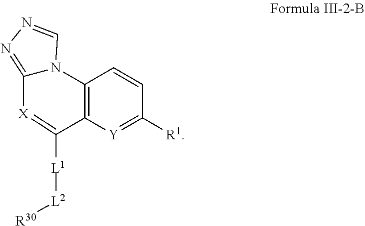

Embodiment A4. The compound of any of Embodiments A1-A3, or a pharmaceutically acceptable salt thereof, wherein (i) R¹ is CN; (ii) R¹ is halogen, e.g., F or Cl; (iii) R¹ is

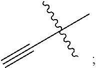 [SMILES: *C#C]

(iv) R¹ is an optionally substituted 5 or 6 membered heteroaryl having 1-4 ring heteroatoms independently selected from O, S, and N, such as an optionally substituted pyrimidinyl or an optionally substituted thiazolyl, for example, R¹ is

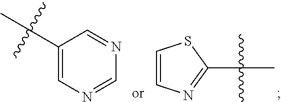

or (v) R¹ is SR^{A1}, wherein R^{A1} is independently optionally substituted C₁₋₄ alkyl, for example, R¹ is SCH₃.
Embodiment A5. The compound of any of Embodiments A1-A4, or a pharmaceutically acceptable salt thereof, wherein L¹ is selected from the following (the bottom attaching point, N or C, is attached to L²):

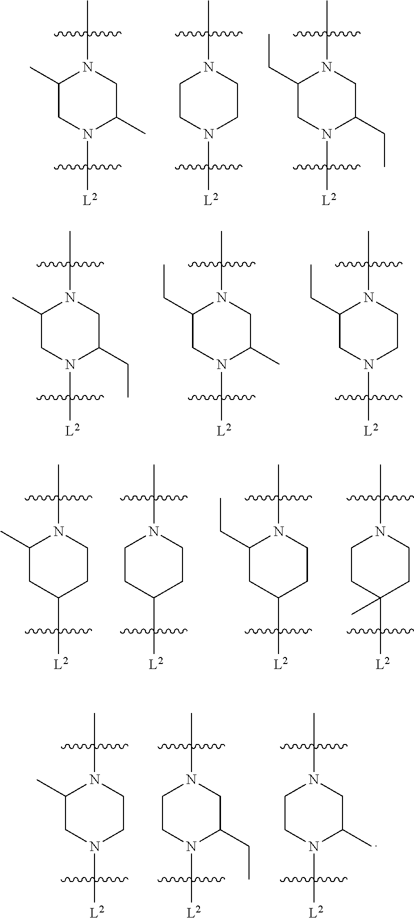

Embodiment A6. The compound of any of Embodiments A1-A4, or a pharmaceutically acceptable salt thereof, wherein L¹ is selected from the following (the bottom attaching point, N or C, is attached to L²):

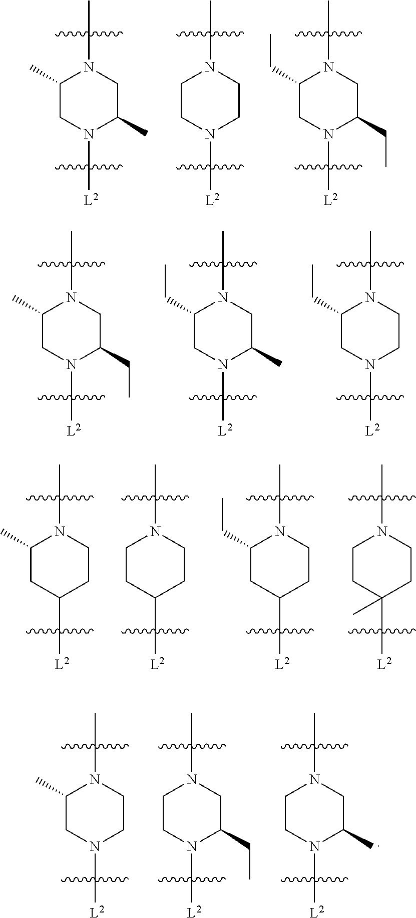

Embodiment A7. The compound of any of Embodiments A1-A4, or a pharmaceutically acceptable salt thereof, characterized as having a structure according to Formula III-2-C:

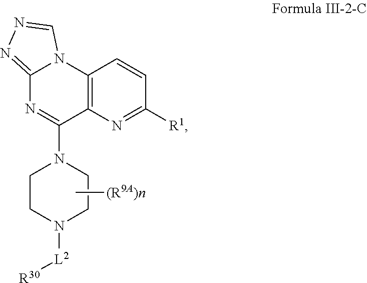

wherein n is 0, 1, 2, or 3, and R^{IA} is defined herein.
Embodiment A8. The compound of Embodiment A7, or a pharmaceutically acceptable salt thereof, characterized as having a structure according to Formula III-2-C-1, III-2-C-2, III-2-C-3, or III-2-C-4:

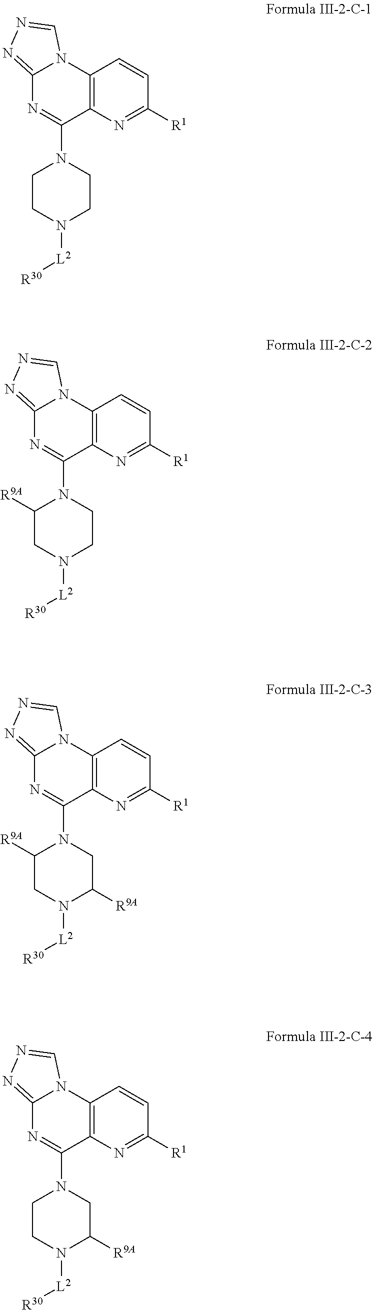

wherein the two R^{9A} in Formula III-2-C-3 are independently selected.
Embodiment A9. The compound of Embodiment A7 or A8, wherein R^{9A} at each occurrence is independently CH₃, CH₂CH₃, CH₂CH₂CH₃, fluorine-substituted C₁₋₃ alkyl (e.g., CF₂H), cyclopropyl, cyclobutyl, CH₂OH, CH₂OCH₃, CH₂OCH₂CH₃, CH₂NH₂, CH₂N₃, or CH₂NHC(O)OCH₃.
Embodiment A10. The compound of any of Embodiments A1-A4, or a pharmaceutically acceptable salt thereof, characterized as having a structure according to Formula III-2-D:

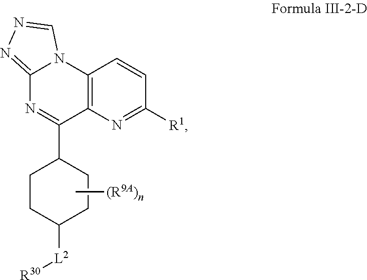

wherein n is 0, 1, 2, or 3, and R^{9A} is defined herein.
Embodiment A11. The compound of Embodiment A10, or a pharmaceutically acceptable salt thereof, characterized as having a structure according to Formula III-2-D-1, III-2-D-2, III-2-D-3, or III-2-D-4:

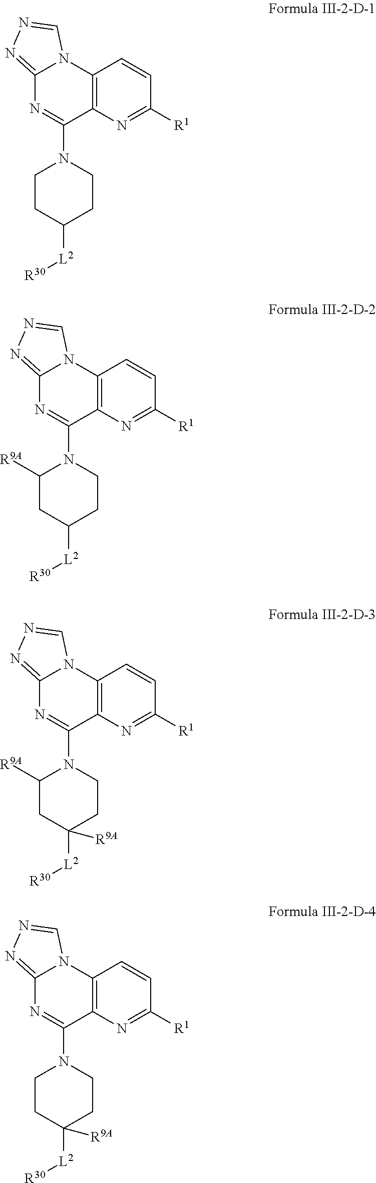

wherein the two R^{9A} in Formula III-2-D-3 are independently selected.
Embodiment A12. The compound of Embodiment A10 or A11, or a pharmaceutically acceptable salt thereof, wherein R^{9A} at each occurrence is independently CH₃, CH₂CH₃, CH₂CH₂CH₃, fluorine-substituted C₁₋₃ alkyl (e.g., CF₂H), cyclopropyl, cyclobutyl, CH₂OH, CH₂OCH₃, CH₂OCH₂CH₃, CH₂NH₂, CH₂N₃, or CH₂NHC(O)OCH₃.
Embodiment A13. The compound of any of Embodiments A1-A12, or a pharmaceutically acceptable salt thereof, characterized as having a structure according to Formula III-2-E-1, III-2-E-2, or III-2-E-3:

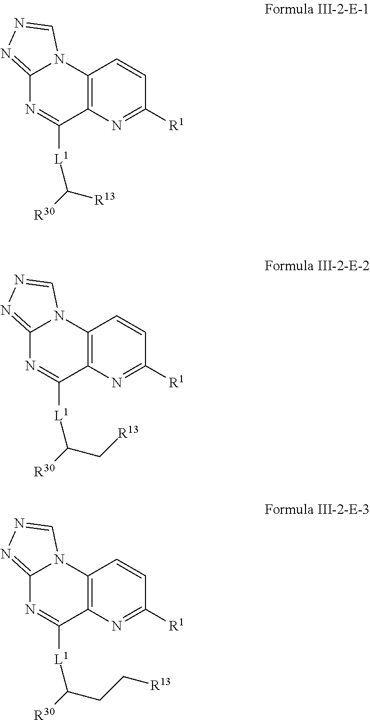

wherein R¹³ is defined herein.
Embodiment A14. The compound of any of Embodiments A1-A12, or a pharmaceutically acceptable salt thereof, characterized as having a structure Formula III-2-E-1a, Formula III-2-E-2a, Formula III-2-E-3a, Formula III-2-E-1b, Formula III-2-E-2b, or Formula III-2-E-3b:

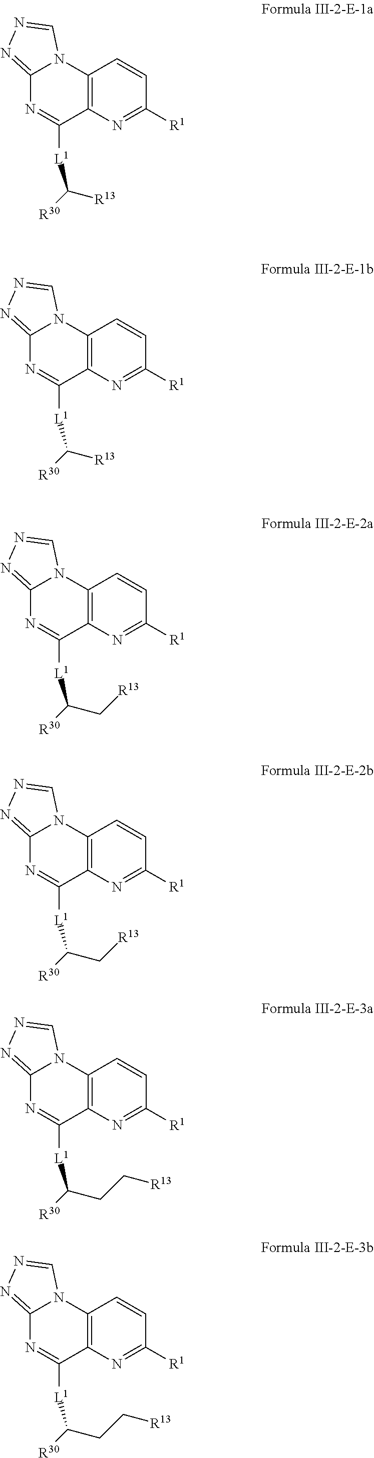

wherein R¹³ is defined herein.
Embodiment A15. The compound of any of Embodiment A13 or A14, or a pharmaceutically acceptable salt thereof, wherein R¹³ is hydrogen, CN, OH, COOH, CONH₂, methoxy, ethoxy, cyclopropyl, cyclobutyl, optionally substituted phenyl, or optionally substituted 5 or 6 membered heteroaryl having 1-3 ring heteroatoms independently selected from N, O, and S, wherein, when substituted, the optionally substituted phenyl or 5 or 6 membered heteroaryl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl, more preferably, R¹³ is hydrogen.
Embodiment A16. The compound of any of Embodiments A1-A12, or a pharmaceutically acceptable salt thereof, wherein L² is absent, O, or C(O).
Embodiment A17. The compound of any of Embodiments A1-A16, or a pharmaceutically acceptable salt thereof, wherein R³⁰ is a phenyl which is substituted with 1-3 (e.g., 1 or 2) R²², wherein R²² at each occurrence is independently halogen (e.g., F, Cl, or Br), CN, OH, R^{M1}, OR^{M1}, SO₂R^{M1} P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.
Embodiment A18. The compound of any of Embodiments A1-A16, or a pharmaceutically acceptable salt thereof, wherein R³⁰ is a phenyl which is substituted with 1-3 (e.g., 1 or 2) R²², wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.
Embodiment A19. The compound of any of Embodiments A1-A16, or a pharmaceutically acceptable salt thereof, wherein R³⁰ is a 5-membered heteroaryl

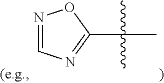 [SMILES: *c1ncno1]

or pyridyl

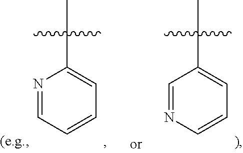 [SMILES: *c1ccccn1 ; *c1cccnc1]

which is substituted with 1-3 (e.g., 1 or 2) R²², as valency permits, wherein R²² at each occurrence is independently halogen (e.g., F, Cl, or Br), CN, OH, R^{M1}, OR^{M1}, SO₂R^{M1} P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.
Embodiment A20. The compound of any of Embodiments A1-A16, or a pharmaceutically acceptable salt thereof, wherein R³⁰ is a 5-membered heteroaryl

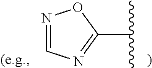 [SMILES: *c1ncno1]

or pyridyl

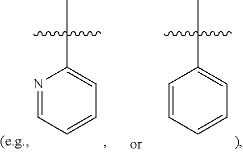 [SMILES: *c1ccccn1 ; *c1ccccc1]

which is substituted with 1-3 (e.g., 1 or 2) R²², as valency permits, wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.
Embodiment A21. The compound of any of Embodiments A1-A16, or a pharmaceutically acceptable salt thereof, wherein R³⁰ has a structure according to S-1-A, S-1-B, or S-1-C:

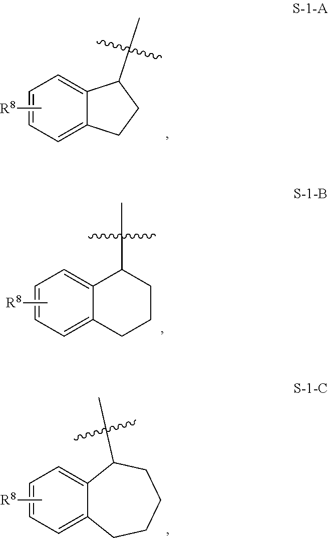

wherein R⁸ is C₁₋₄ alkyl optionally substituted with 1-3 F (such as CH₃, CF₃, etc.), C₁₋₄ alkoxy optionally substituted with 1-3 F (such as OCH₃, OCF₃, etc.), SCF₃, SF₅, cyclopropyl, cyclobutyl, or

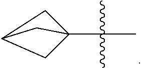 [SMILES: *C12CC(C1)C2]

Embodiment A22. The compound of any of Embodiments A1-A16, or a pharmaceutically acceptable salt thereof, wherein R³⁰ is selected from:

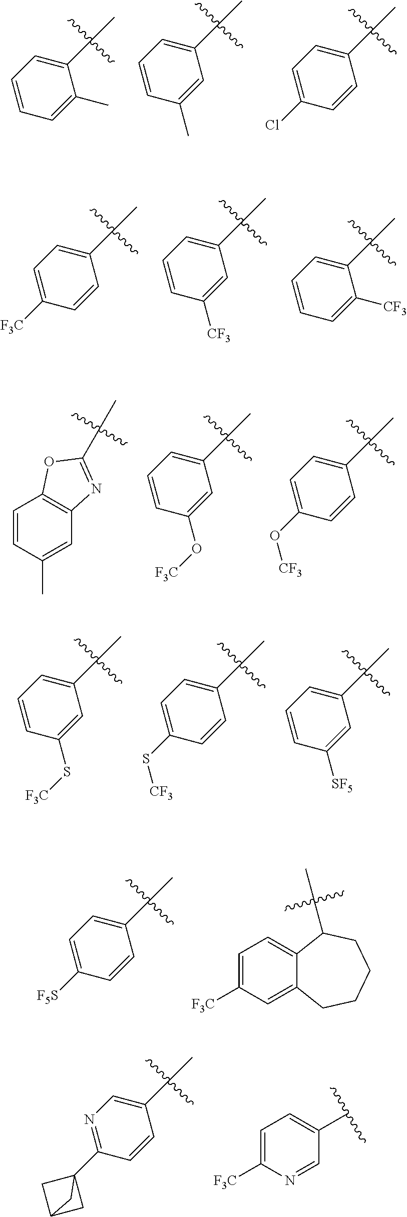

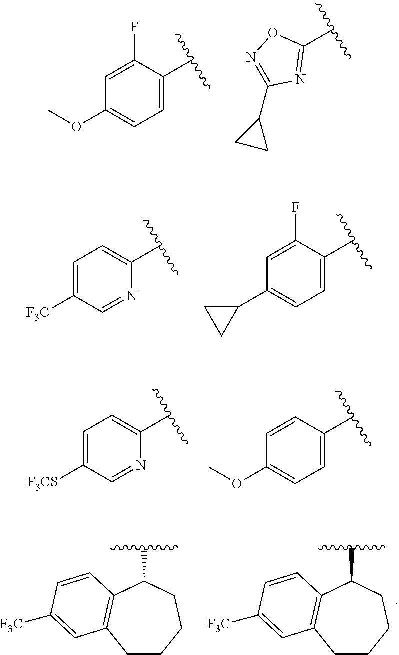

Embodiment A23. The compound of any of Embodiments A1-A4, or a pharmaceutically acceptable salt thereof, wherein L¹-L²-R³⁰ is selected from the following:

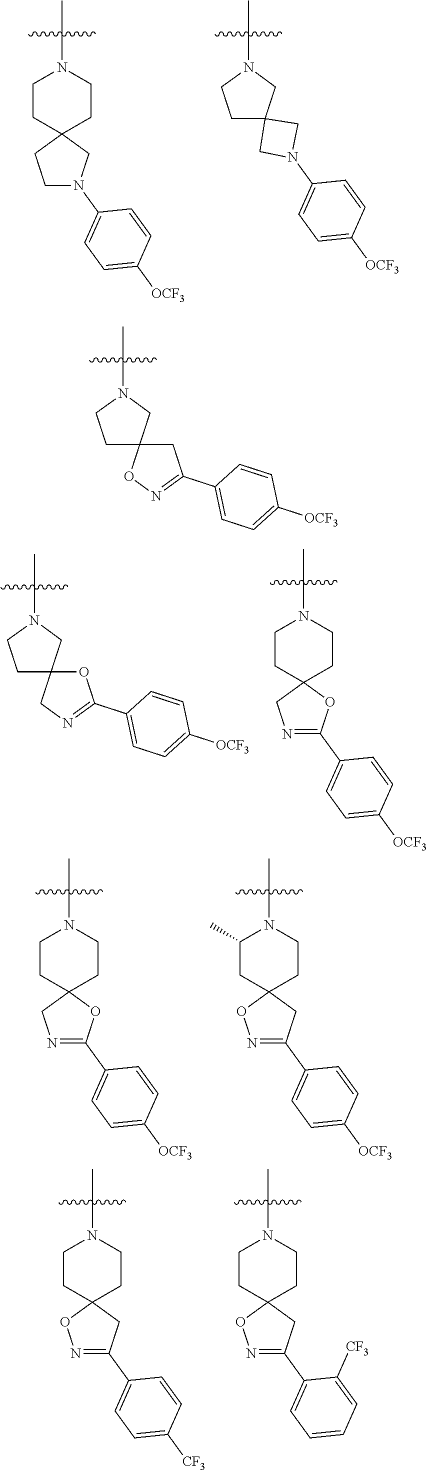

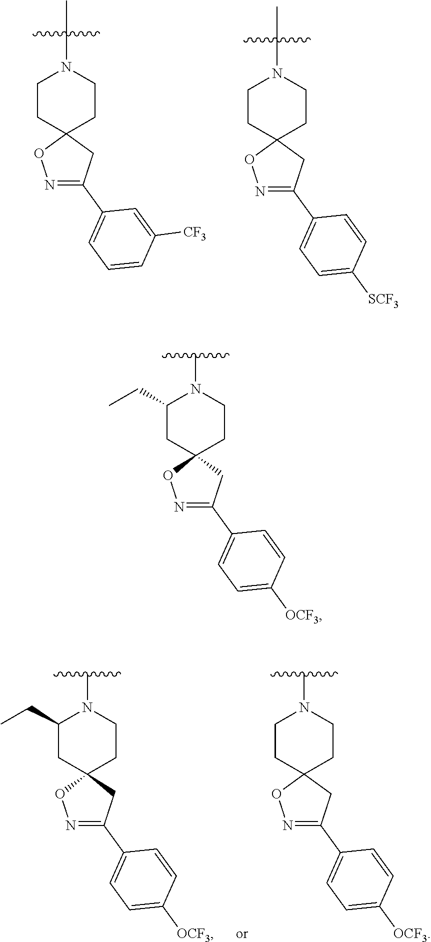

Embodiment A24. The compound of any of Embodiments A1-A4, or a pharmaceutically acceptable salt thereof, wherein L¹-L²-R³⁰ is selected from the following:

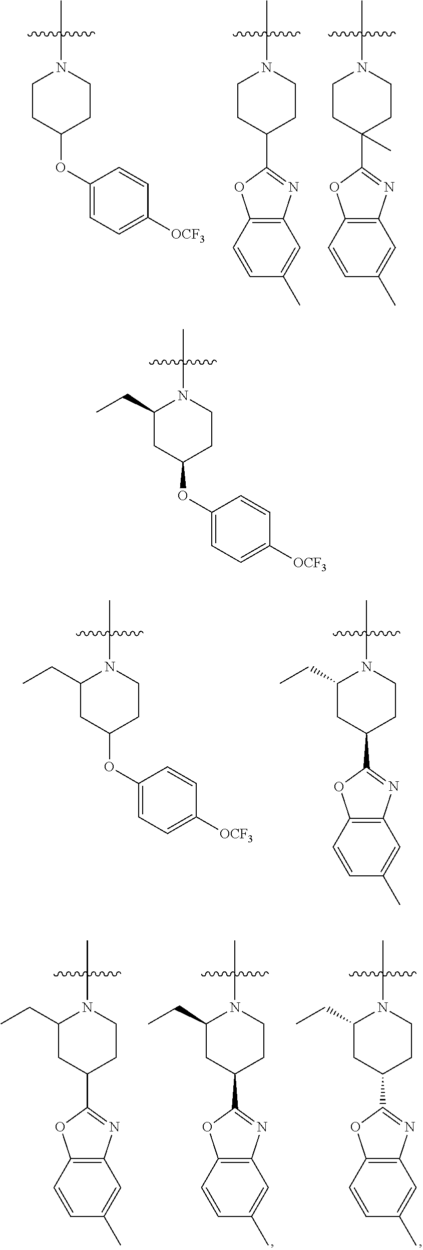

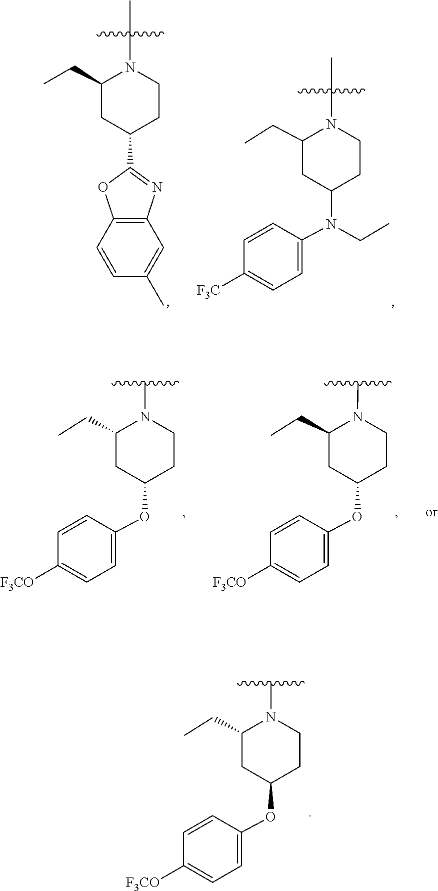 [SMILES: *N1CC[C@H](c2nc3cc(C)ccc3o2)C[C@H]1CC ; *N1CCC(N(CC)c2ccc(C(F)(F)F)cc2)CC1CC ; *N1CC[C@H](Oc2ccc(OC(F)(F)F)cc2)C[C@@H]1CC ; *N1CC[C@H](Oc2ccc(OC(F)(F)F)cc2)C[C@H]1CC ; *N1CC[C@@H](Oc2ccc(OC(F)(F)F)cc2)C[C@@H]1CC]

Embodiment A25. The compound of any of Embodiments A1-A4, or a pharmaceutically acceptable salt thereof, wherein L¹-L²-R³⁰ is selected from the following:

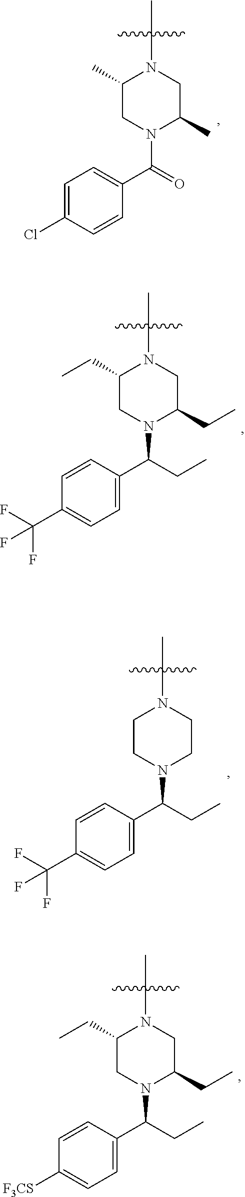

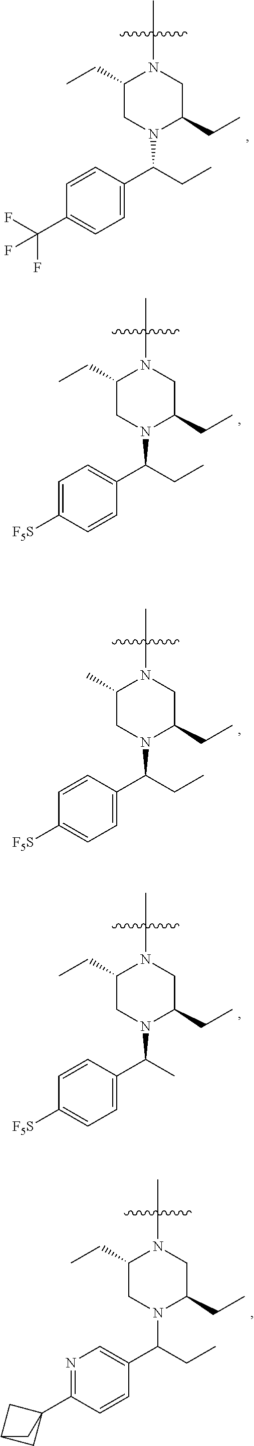

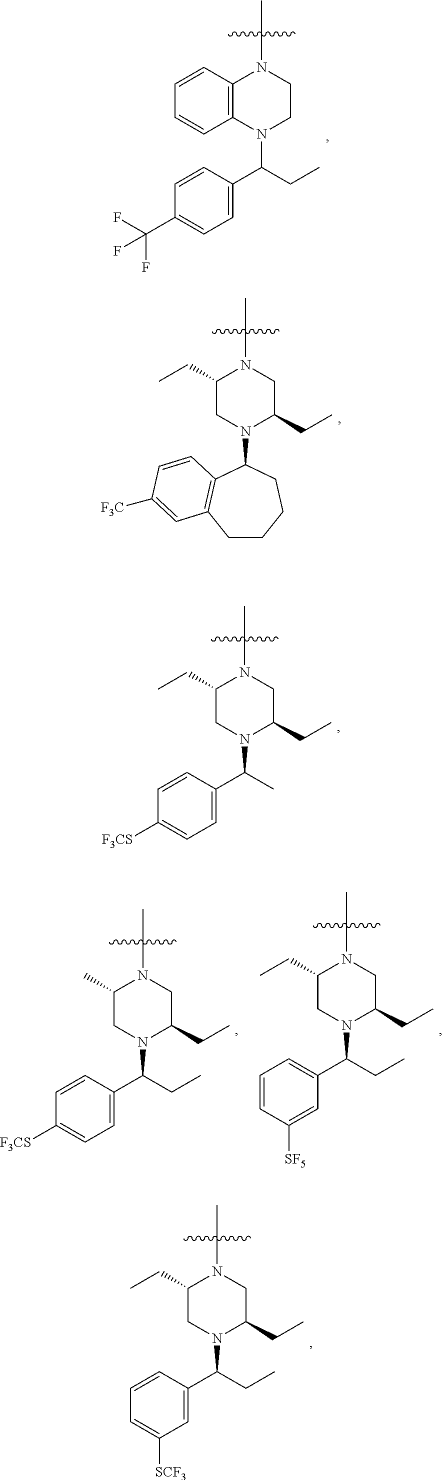

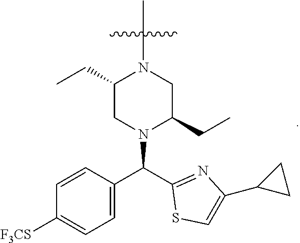

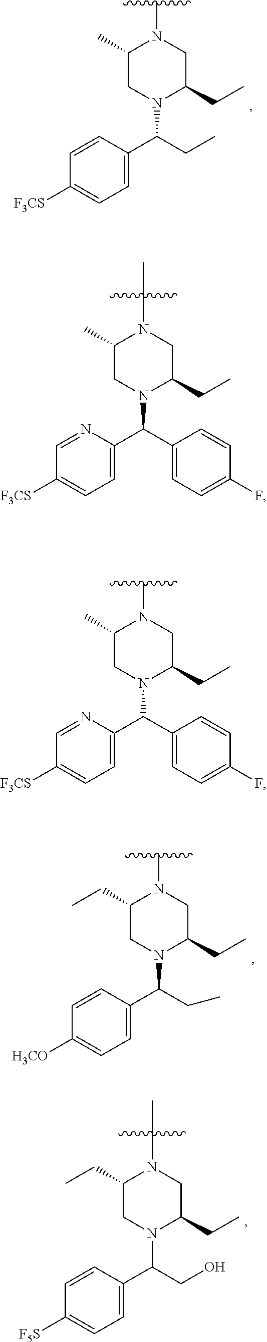

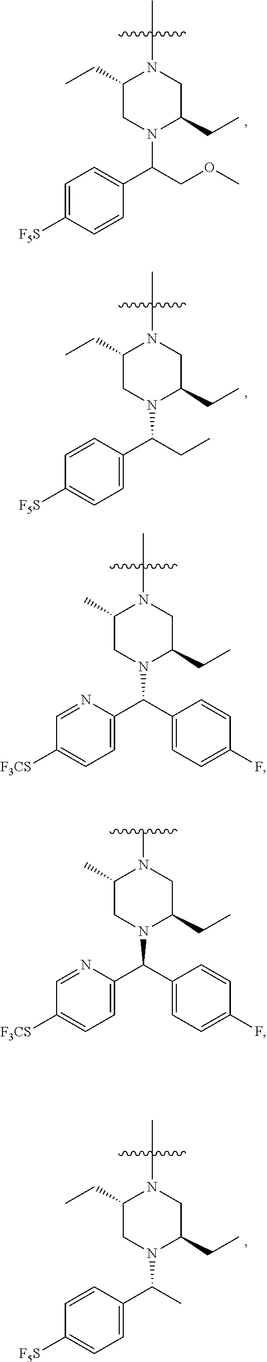

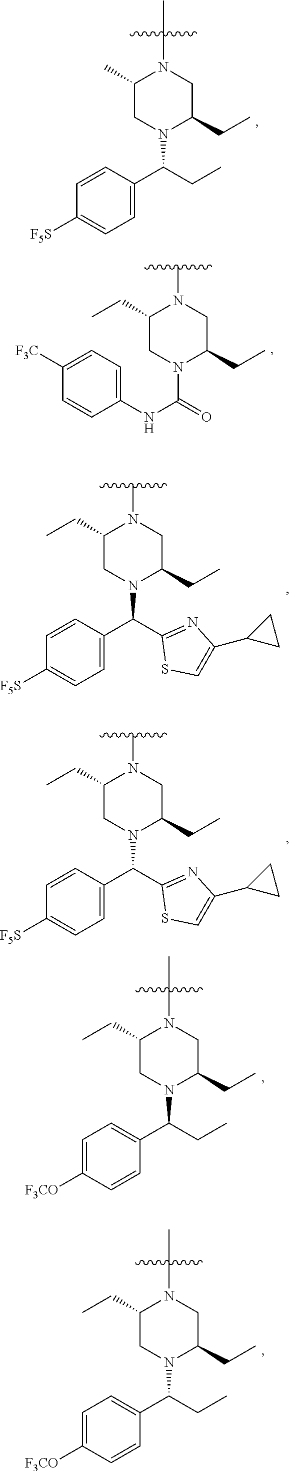

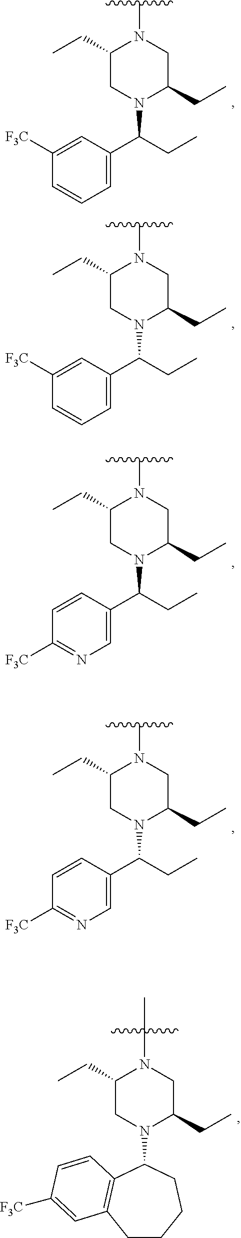 [SMILES: *N1C[C@@H](CC)N([C@@H](CC)c2cccc(C(F)(F)F)c2)C[C@@H]1CC ; *N1C[C@@H](CC)N([C@H](CC)c2cccc(C(F)(F)F)c2)C[C@@H]1CC ; *N1C[C@@H](CC)N([C@@H](CC)c2ccc(C(F)(F)F)nc2)C[C@@H]1CC ; *N1C[C@@H](CC)N([C@H](CC)c2ccc(C(F)(F)F)nc2)C[C@@H]1CC ; *N1C[C@@H](CC)N([C@@H]2CCCCc3cc(C(F)(F)F)ccc32)C[C@@H]1CC]

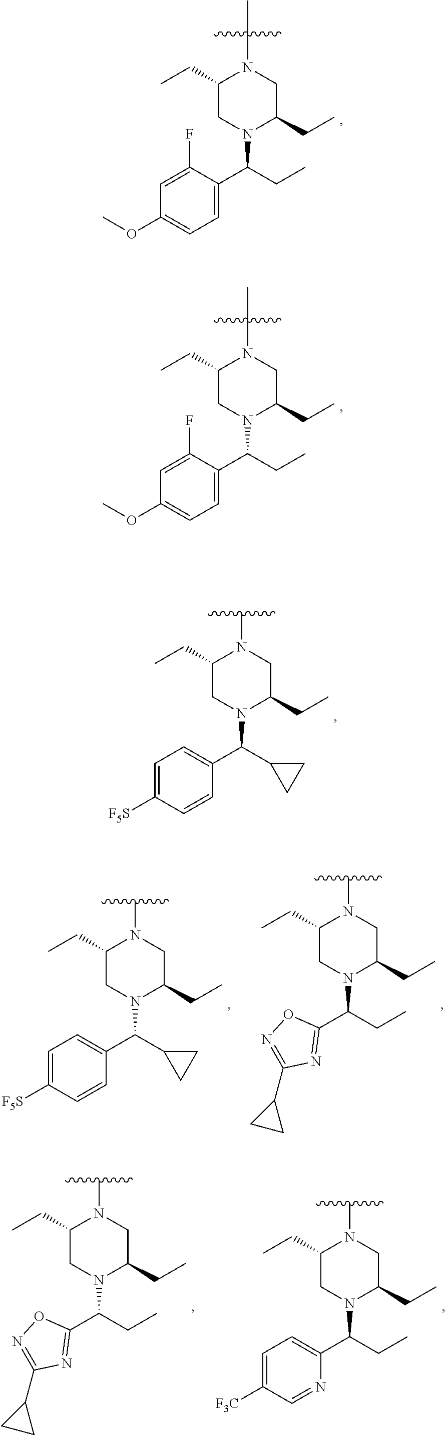

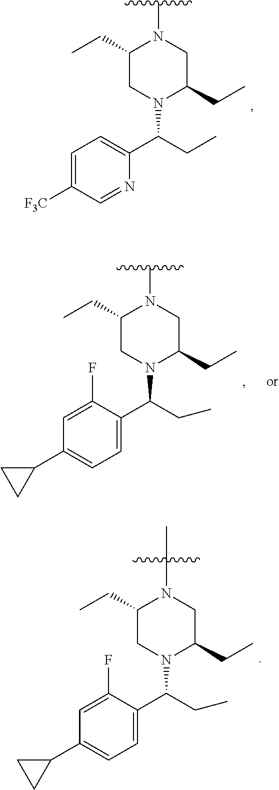 [SMILES: *N1C[C@@H](CC)N([C@H](CC)c2ccc(C(F)(F)F)cn2)C[C@@H]1CC ; *N1C[C@@H](CC)N([C@@H](CC)c2ccc(C3CC3)cc2F)C[C@@H]1CC ; *N1C[C@@H](CC)N([C@H](CC)c2ccc(C3CC3)cc2F)C[C@@H]1CC]

Embodiment A26. The compound of any of Embodiments A1-A4, or a pharmaceutically acceptable salt thereof, wherein L¹-L²-R³⁰ is selected from the following:

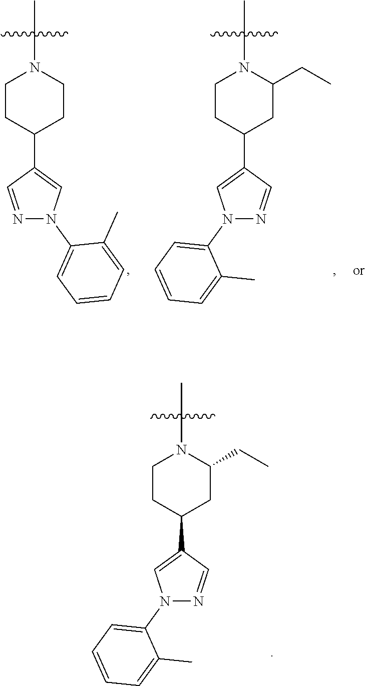

In some embodiments, the present disclosure provides the following enumerated exemplified Embodiments B1-B21:
Embodiment B1. A compound of Formula III-3, or a pharmaceutically acceptable salt thereof,

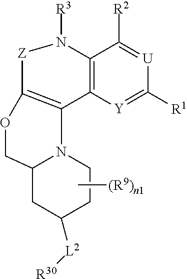

wherein the variables Y, U, Z, R¹, R², R³, R⁹, n1, L², and R³⁰ are defined herein.
Embodiment B2. The compound of Embodiment B1, or a pharmaceutically acceptable salt thereof, characterized as having a structure according to Formula III-3-A:

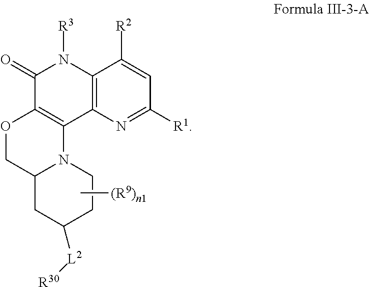

Embodiment B3. A compound of Formula III-4, or a pharmaceutically acceptable salt thereof,

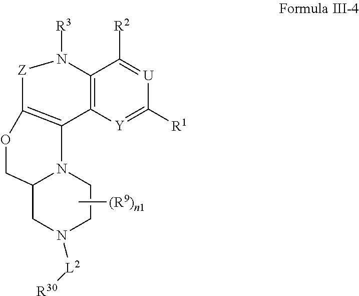

wherein the variables Y, U, Z, R¹, R², R³, R⁹, n1, L², and R³⁰ are defined herein.
Embodiment B4. The compound of Embodiment B3, or a pharmaceutically acceptable salt thereof, characterized as having a structure according to Formula III-4-A:

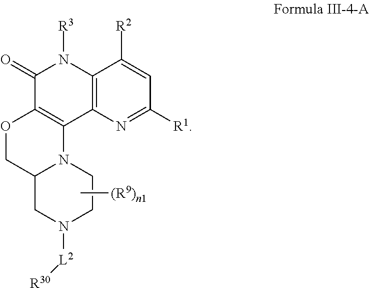

Embodiment B5. The compound of Embodiment B3, or a pharmaceutically acceptable salt thereof, characterized as having a structure according to Formula III-4-B:

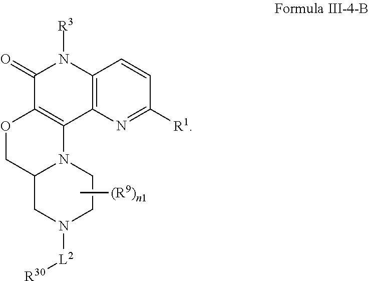

Embodiment B6. The compound of Embodiment B5, or a pharmaceutically acceptable salt thereof, characterized as having a structure according to Formula III-4-C or III-4-D:

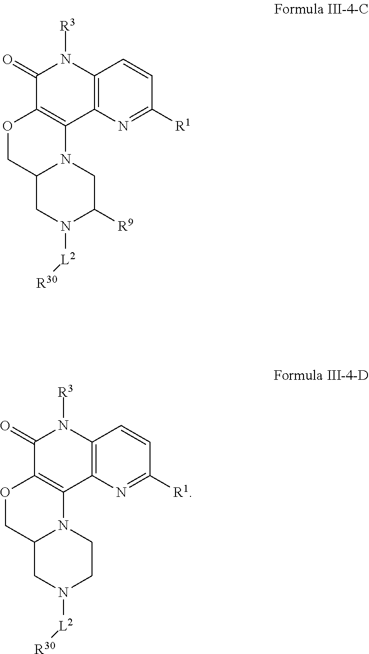

Embodiment B7. The compound of Embodiment B6, or a pharmaceutically acceptable salt thereof, characterized as having a structure according to Formula III-4-C-1, III-4-C-2, III-4-C-3, III-4-C-4, III-4-D-1, or III-4-D-2:

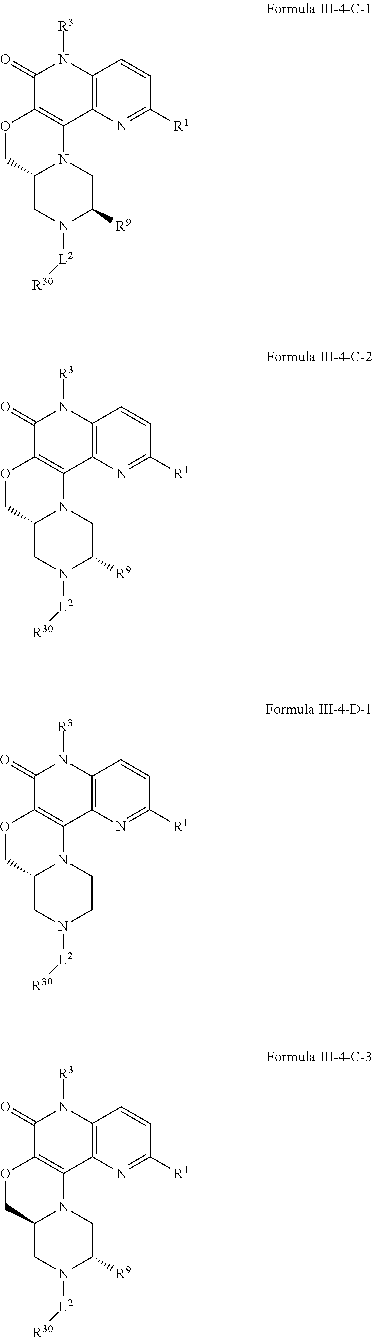

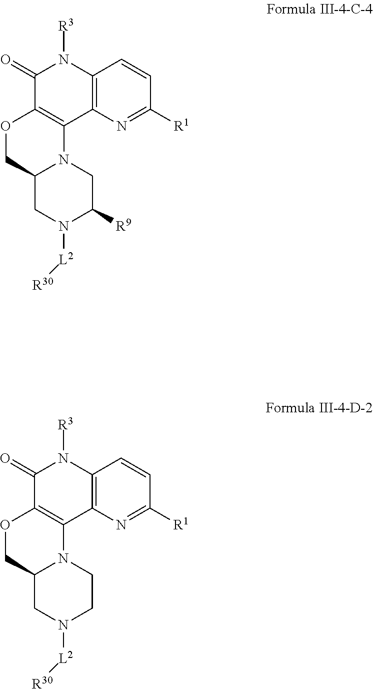

Embodiment B8. The compound of any of Embodiments B1-B7, or a pharmaceutically acceptable salt thereof, wherein R³ is an optionally substituted C₁₋₄ alkyl, such as CH₃, CD₃ etc.
Embodiment B9. The compound of any of Embodiments B1-B8, or a pharmaceutically acceptable salt thereof, wherein (i) R¹ is CN; (ii) R¹ is halogen, e.g., F or Cl; (iii) R¹ is

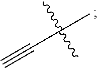 [SMILES: *C#C]

(iv) R¹ is an optionally substituted 5 or 6 membered heteroaryl having 1-4 ring heteroatoms independently selected from O, S, and N, such as an optionally substituted pyrimidinyl or an optionally substituted thiazolyl, for example, R¹ is

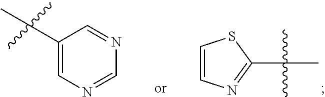 [SMILES: *c1cncnc1 ; *c1nccs1]

or (v) R¹ is SR^{A1}, wherein R^{A1} is independently optionally substituted C₁₋₄ alkyl, for example, R¹ is SCH₃.
Embodiment B10. The compound of any of Embodiments B1-B9, or a pharmaceutically acceptable salt thereof, wherein R⁹ at each occurrence is independently CH₃, CH₂CH₃, CH₂CH₂CH₃, fluorine-substituted C₁₋₃ alkyl (e.g., CF₂H), cyclopropyl, cyclobutyl, CH₂OH, CH₂OCH₃, CH₂OCH₂CH₃, CH₂NH₂, CH₂N₃, or CH₂NHC(O)OCH₃
Embodiment B11. The compound of any of Embodiments B1-B10, or a pharmaceutically acceptable salt thereof, wherein L² is an optionally substituted C₁₋₄ alkylene.
Embodiment B12. The compound of any of Embodiments B1-B10, or a pharmaceutically acceptable salt thereof, wherein L² is selected from the following:

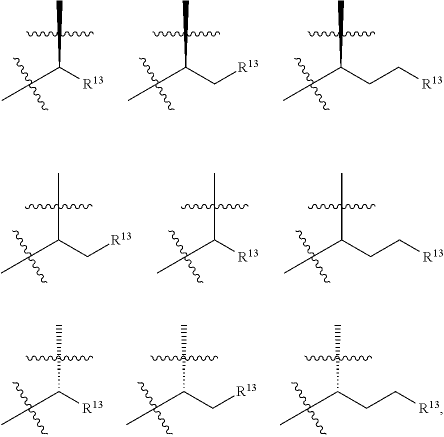

wherein R¹³ is defined herein.
Embodiment B13. The compound of Embodiment B12, or a pharmaceutically acceptable salt thereof, wherein R¹³ is hydrogen, CN, OH, COOH, CONH₂, methoxy, ethoxy, cyclopropyl, cyclobutyl, optionally substituted phenyl, or optionally substituted 5 or 6 membered heteroaryl having 1-3 ring heteroatoms independently selected from N, O, and S, wherein, when substituted, the optionally substituted phenyl or 5 or 6 membered heteroaryl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl, more preferably, R¹³ is hydrogen.
Embodiment B14. The compound of any of Embodiments B1-B10, or a pharmaceutically acceptable salt thereof, wherein L² is absent, O, or C(O).
Embodiment B15. The compound of any of Embodiments B1-B14, or a pharmaceutically acceptable salt thereof, wherein R³⁰ is a phenyl which is substituted with 1-3 (e.g., 1 or 2) R²², wherein R²² at each occurrence is independently halogen (e.g., F, Cl, or Br), CN, OH, R^{M1}, OR^{M1}, SO₂R^{M1} P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.
Embodiment B16. The compound of any of Embodiments B1-B14, or a pharmaceutically acceptable salt thereof, wherein R³⁰ is a phenyl which is substituted with 1-3 (e.g., 1 or 2) R²², wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.
Embodiment B17. The compound of any of Embodiments B1-B14, or a pharmaceutically acceptable salt thereof, wherein R³⁰ is a 5-membered heteroaryl

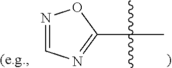 [SMILES: *c1ncno1]

or pyridyl

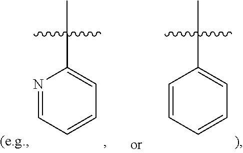 [SMILES: *c1ccccn1 ; *c1ccccc1]

which is substituted with 1-3 (e.g., 1 or 2) R²², as valency permits, wherein R²² at each occurrence is independently halogen (e.g., F, Cl, or Br), CN, OH, R^{M1}, OR^{M1}, SO₂R^{M1} P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.
Embodiment B18. The compound of any of Embodiments B1-B14, or a pharmaceutically acceptable salt thereof, wherein R³⁰ is a 5-membered heteroaryl

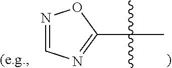 [SMILES: *c1ncno1]

or pyridyl

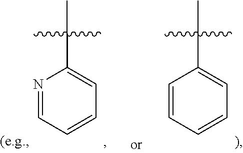 [SMILES: *c1ccccn1 ; *c1ccccc1]

which is substituted with 1-3 (e.g., 1 or 2) R²², as valency permits, wherein R²² at each occurrence is independently F, Cl, CN, R^{M2}, OR^{M2}. SR^{M2}, or SF₅, wherein R^{M2} at each occurrence is independently a C₁₋₄ alkyl optionally substituted with 1-3 F, such as CF₃.
Embodiment B19. The compound of any of Embodiments B1-B14, or a pharmaceutically acceptable salt thereof, wherein R³⁰ has a structure according to S-1-A, S-1-B, or S-1-C:

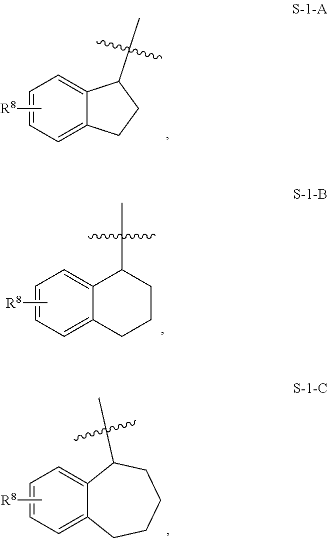

wherein R⁸ is C₁₋₄ alkyl optionally substituted with 1-3 F (such as CH₃, CF₃, etc.), C₁₋₄ alkoxy optionally substituted with 1-3 F (such as OCH₃, OCF₃, etc.), SCF₃, SF₅, cyclopropyl, cyclobutyl, or.

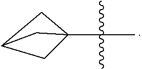 [SMILES: *C12CC(C1)C2]

Embodiment B20. The compound of any of Embodiments B1-B14, or a pharmaceutically acceptable salt thereof, wherein R³⁰ is selected from:

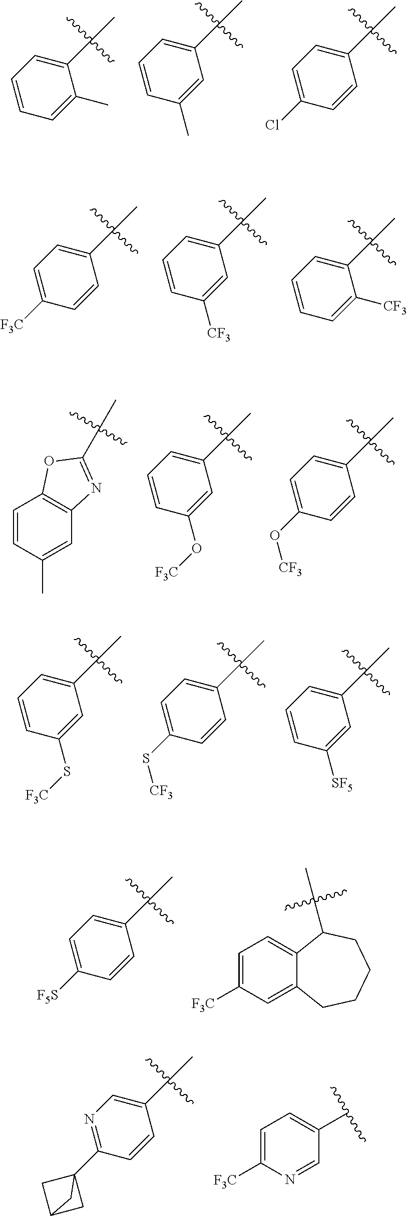

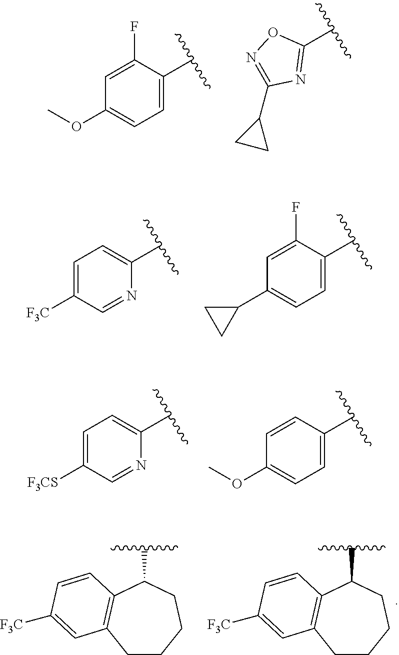

Embodiment B21. The compound of any of Embodiments B1-B10, or a pharmaceutically acceptable salt thereof, wherein L²-R³⁰ is selected from:

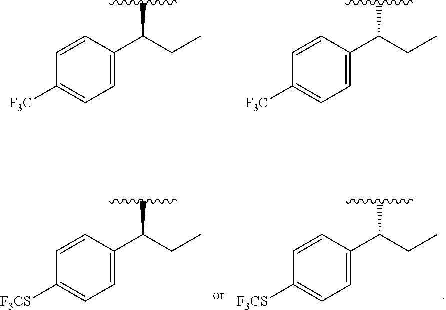

### Formula IV

In some embodiments, the present disclosure provides a compound of Formula IV, or a pharmaceutically acceptable salt thereof:

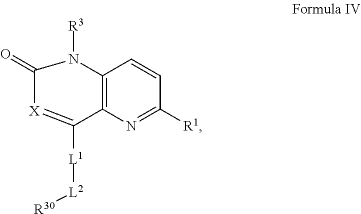

wherein:
   R¹ is hydrogen, halogen (e.g., F, Cl, or Br), CN, OH, NH₂, COOH, CONH₂, R^{A}, OR^{A}, NH(R^{A}), N(R^{A})₂, COOR^{A}, CONH(R^{A}), CON(R^{A})₂, SR^{A}, SOR^{A}, SO₂R^{A}, or P(O)(R^{A})₂, wherein R^{A} at each occurrence is independently optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
   R³ is hydrogen, optionally substituted C₁₋₄ alkyl, optionally substituted C₃₋₆ cycloalkyl, or optionally substituted 4-7 membered heterocyclyl (such as a saturated 4-7 membered heterocyclyl);
   X is N or CR⁵, wherein R⁵ is hydrogen, halogen, CN, CONH₂, COOH, CONH(R^{C}), CON(R^{C})₂, OR^{C}, NO₂, or COOR^{C}, wherein R^{C} at each occurrence is independently an optionally substituted C₁₋₄ alkyl;
   L¹ and L² are characterized in that:
   i) L¹ is a 6-12 membered heterocyclylene having two or more rings and 1-4 ring heteroatoms each independently O, N, or S, wherein the heterocyclylene is optionally substituted; and L² is absent, O, C(O), an optionally substituted C₁₋₄ alkylene, an optionally substituted C₂₋₄ alkenylene, an optionally substituted C₁₋₄ alkynylene, optionally substituted C₁₋₄ heteroalkylene, or an optionally substituted 3-7 membered ring;
   ii) L¹ is a 4-7 membered monocyclic heterocyclylene having 1-2 ring heteroatoms each independently O, N, or S, wherein the monocyclic heterocyclylene is optionally substituted; and L² is absent, O, C(O), or an optionally substituted 3-7 membered ring; or
   iii) L¹ is —N(R^{E})—, wherein R^{E} is optionally substituted C₁₋₄ alkyl or optionally substituted 3-7 membered ring; and L² is absent or an optionally substituted 3-7 membered ring; and
   R³⁰ is hydrogen, an optionally substituted phenyl, optionally substituted 5 or 6 membered heteroaryl, or optionally substituted 8-12 membered ring having two or more rings.

In some embodiments, the compound of Formula IV can have stereoisomer(s). In such embodiments, the compound of Formula IV can exist in the form of an individual enantiomer, diastereomer, atropisomer, and/or geometric isomer, as applicable, or a mixture of stereoisomers, including racemic mixtures and mixtures enriched in one or more stereoisomers. In some embodiments, when applicable, the compound of Formula IV can exist as a mixture of a pair of enantiomers in any ratio, including a racemic mixture with a ratio of 1:1. In some embodiments, when applicable, the compound of Formula IV can exist as an isolated or enriched individual enantiomer substantially free (e.g., with less than 20%, less than 10%, less than 5%, less than 1%, by weight, by HPLC or SFC area, or both, or with a non-detectable amount) of the other enantiomer.

Unless otherwise specified or contrary from context, the applicable variables R¹, R³, L¹, L², R³⁰, and X suitable for Formula IV, include any of those respective definitions in connection with Formula I (e.g., Formula I-1, I-2, I-3, I-4, I-5, I-6, I-A, I-B, I-C, I-A-1, I-B-1, I-C-1, I-D, I-E, I-L-1, I-L-2, I-L-3, I-L-4, I-L-5, I-L-6, I-L-7, I-L-8, I-L-9, I-L-10, or I-L-11), Formula II-1 to II-7, and Formula III-1 to III-9, as described and preferred herein in any combinations. For example, in some embodiments, R¹ is CN. In some embodiments, R¹ can be characterized in that (i) R¹ is halogen, e.g., F or Cl; (ii) R¹ is

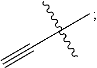 [SMILES: *C#C]

(iii) R¹ is an optionally substituted 5 or 6 membered heteroaryl having 1-4 ring heteroatoms independently selected from O, S, and N, such as an optionally substituted pyrimidinyl or an optionally substituted thiazolyl, for example, R¹ is

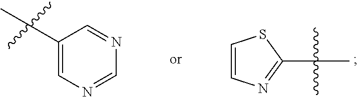 [SMILES: *c1cncnc1 ; *c1nccs1]

or (iv) R¹ is SR^{A1}, wherein R^{A1} is independently optionally substituted C₁₋₄ alkyl, for example, R¹ is SCH₃. In some embodiments, R³ is an optionally substituted C₁₋₄ alkyl, such as CH₃, CD₃ etc. In some embodiments, X is N. In some embodiments, X is CR⁵, wherein R⁵ is CN. In some embodiments, X is CR⁵, wherein R⁵ is CONH₂, COOH, CONH(R^{C1}), CON(R^{C1})₂, or COOR^{C1}, wherein Rei at each occurrence is independently a C₁₋₄ alkyl, such as methyl.

In some preferred embodiments, L¹ in Formula IV can be a bicyclic heterocyclylene selected from the following (the bottom attaching point, N or C, is attached to L²):

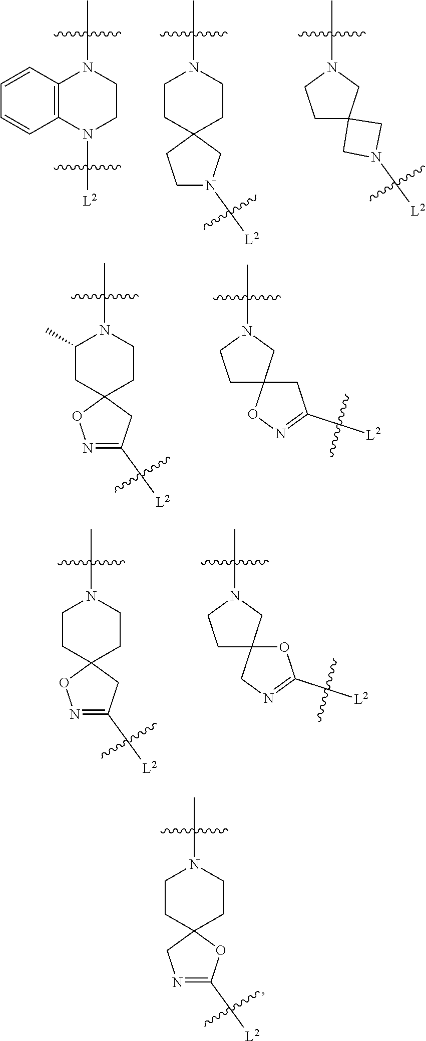

or L¹ is selected from

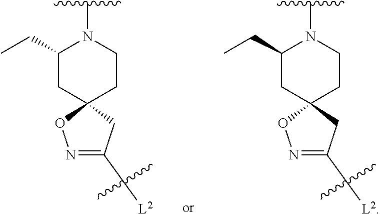

In some embodiments, L² is absent. In some embodiments, L² can be O or C(O).

In some embodiments, in Formula IV, R³⁰ is optionally substituted phenyl, such as a phenyl which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl. For example, in some embodiments, R³⁰ is a phenyl which is substituted with 1-3 (e.g., 1 or 2) R²², wherein R²² at each occurrence is independently halogen (e.g., F, Cl, or Br), CN, OH, R^{M1}, OR^{M1}, SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. More preferably, R³⁰ is a phenyl which is substituted with 1-3 (e.g., 1 or 2) R²², wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

In some embodiments, in Formula IV, R³⁰ is optionally substituted 5 or 6-membered heteroaryl, such as a pyridyl

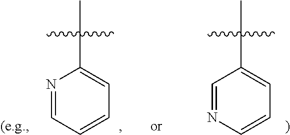 [SMILES: *c1ccccn1 ; *c1cccnc1]

which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from 0, S, and N, or optionally substituted 4-7 membered heterocyclyl. For example, in some embodiments, R³⁰ is a 5-membered heteroaryl

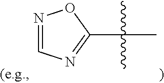 [SMILES: *c1ncno1]

or pyridyl

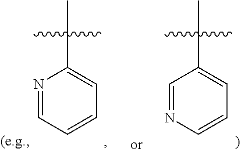 [SMILES: *c1ccccn1 ; *c1cccnc1]

which is substituted with 1-3 (e.g., 1 or 2) R²², as valency permits, wherein R²² at each occurrence is independently halogen (e.g., F, Cl, or Br), CN, OH, R^{M1}, OR^{M1} SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. More preferably, R³⁰ is a 5-membered heteroaryl

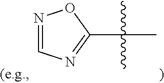 [SMILES: *c1ncno1]

or pyridyl

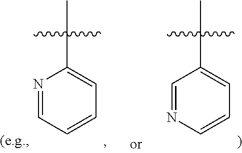 [SMILES: *c1ccccn1 ; *c1cccnc1]

which is substituted with 1-3 (e.g., 1 or 2) R²², as valency permits, wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

In some embodiments, R³⁰ in Formula IV can have any of the definitions described for Ring A and R⁴ in connection with Formula I and the subformulae.

For example, in some embodiments, R³⁰ can have a structure according to S-1-A, S-1-B, or S-1-C:

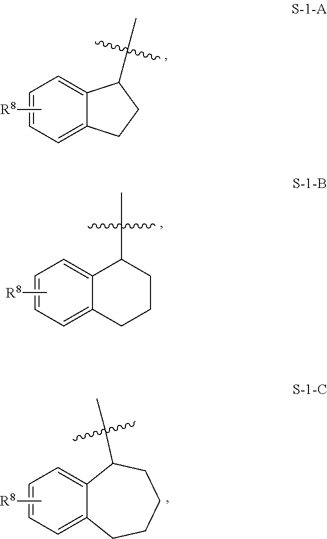

wherein R⁸ is C₁₋₄ alkyl optionally substituted with 1-3 F (such as CH₃, CF₃, etc.), C₁₋₄ alkoxy optionally substituted with 1-3 F (such as OCH₃, OCF₃, etc.), SCF₃, SF₅, cyclopropyl, cyclobutyl, or

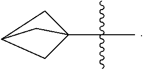 [SMILES: *C12CC(C1)C2]

In some preferred embodiments, R³⁰ in Formula IV can be selected from the following:

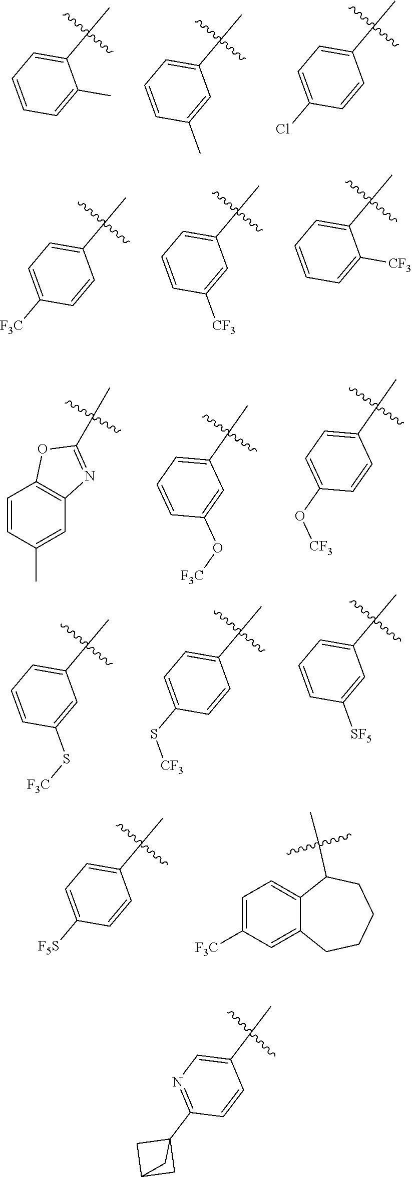

or R³⁰ is selected from:

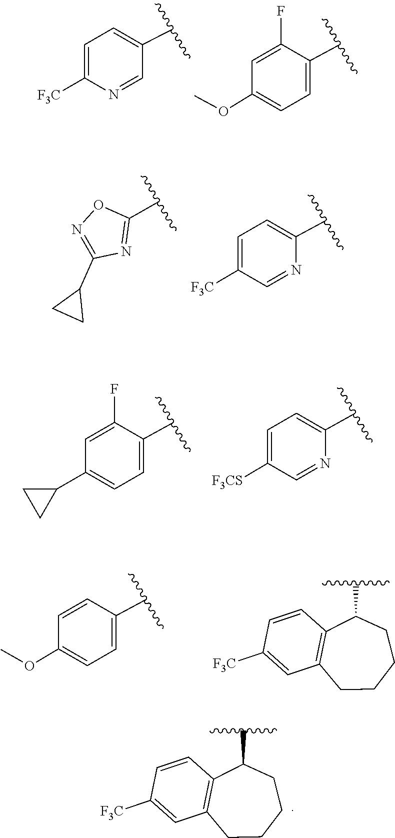

In some embodiments, in Formula IV, L¹, L² and R³⁰ together can be selected from:

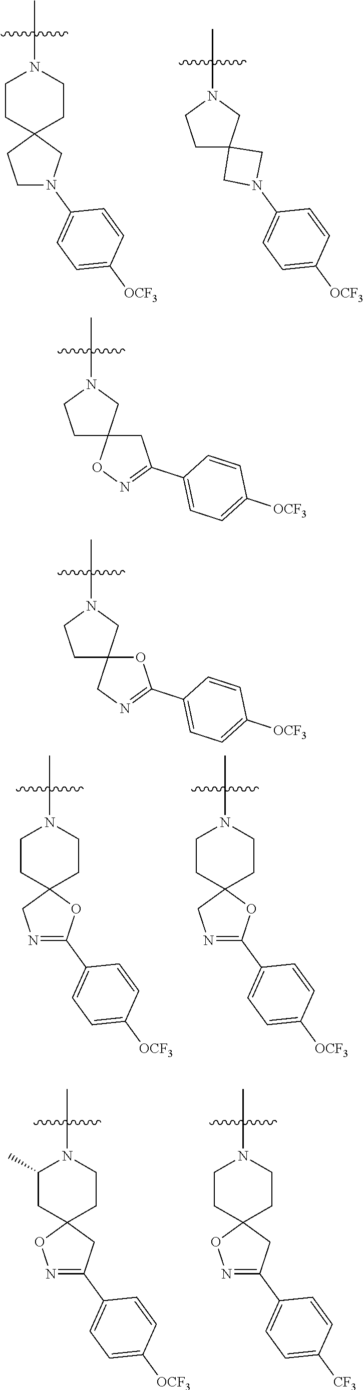

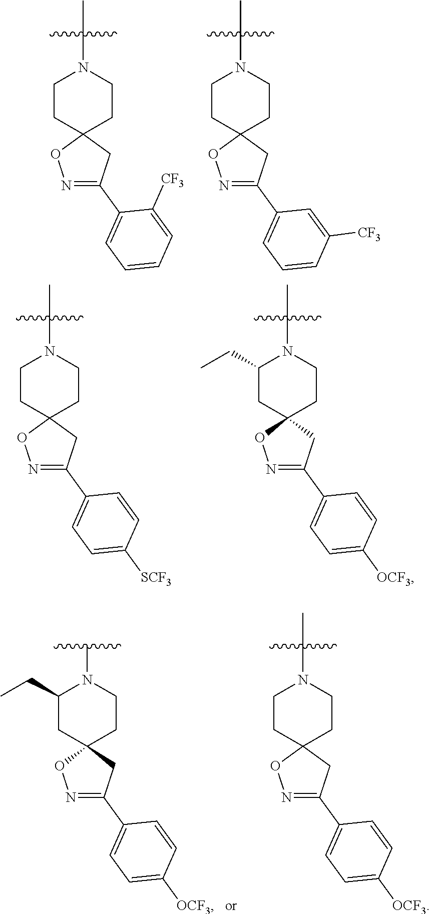

In some embodiments, in Formula IV, L¹, L², and R³⁰ together can be selected from:

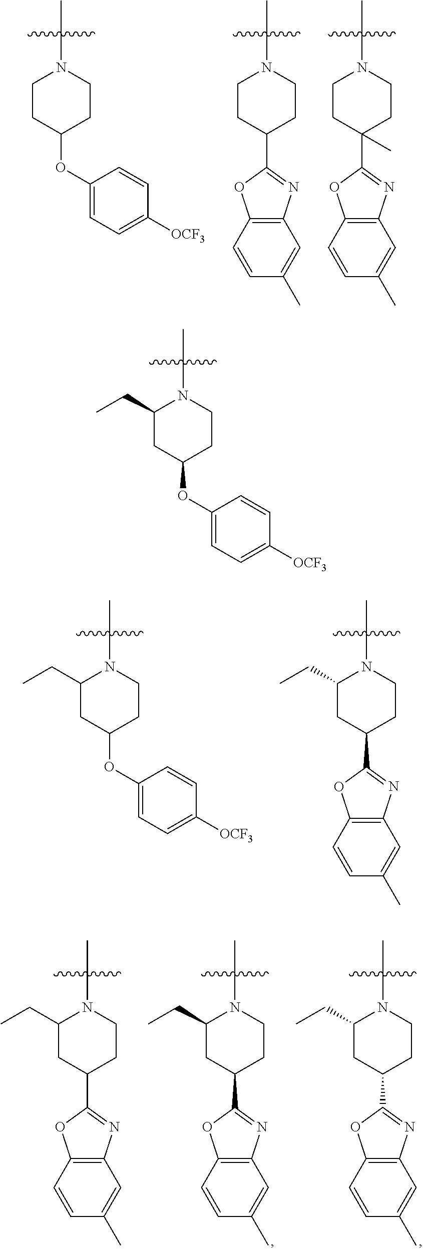

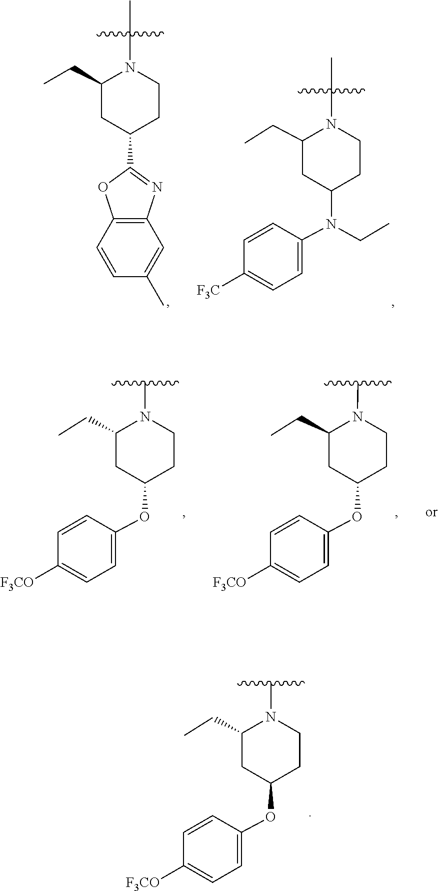 [SMILES: *N1CC[C@H](c2nc3cc(C)ccc3o2)C[C@H]1CC ; *N1CCC(N(CC)c2ccc(C(F)(F)F)cc2)CC1CC ; *N1CC[C@H](Oc2ccc(OC(F)(F)F)cc2)C[C@@H]1CC ; *N1CC[C@H](Oc2ccc(OC(F)(F)F)cc2)C[C@H]1CC ; *N1CC[C@@H](Oc2ccc(OC(F)(F)F)cc2)C[C@@H]1CC]

In some embodiments, in Formula IV, L¹, L², and R³⁰ together can be selected from:

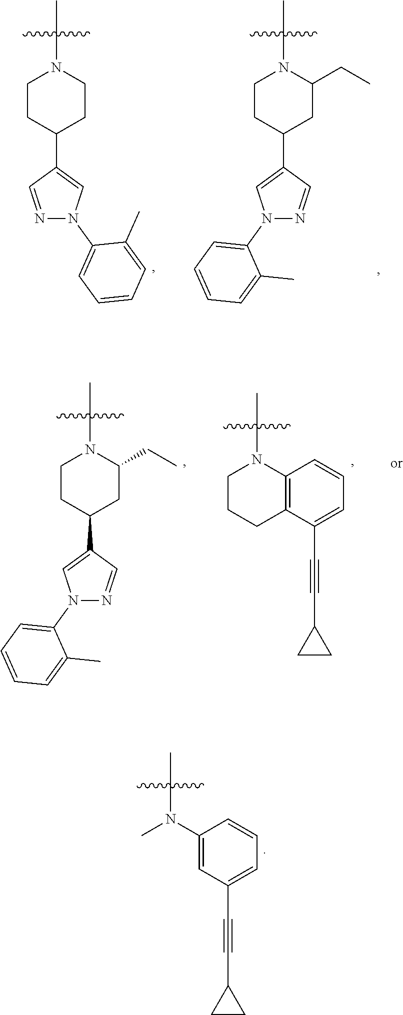 [SMILES: *N1CCC(c2cnn(-c3ccccc3C)c2)CC1 ; *N1CCC(c2cnn(-c3ccccc3C)c2)CC1CC ; *N1CC[C@H](c2cnn(-c3ccccc3C)c2)C[C@H]1CC ; *N1CCCc2c(C#CC3CC3)cccc21 ; *N(C)c1cccc(C#CC2CC2)c1]

### Formula V

In some embodiments, the present disclosure provides a compound of Formula V, or a pharmaceutically acceptable salt thereof:

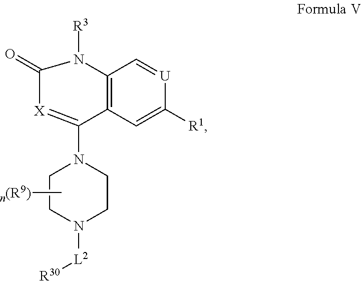

wherein:
   R¹ is hydrogen, halogen (e.g., F, Cl, or Br), CN, OH, NH₂, COOH, CONH₂, R^{A}, OR^{A}, NH(R^{A}), N(R^{A})₂, COOR^{A}, CONH(R^{A}), CON(R^{A})₂, SR^{A}, SOR^{A}, SO₂R^{A}, or P(O)(R^{A})₂, wherein R^{A} at each occurrence is independently optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
   R³ is hydrogen, optionally substituted C₁₋₄ alkyl, optionally substituted C₃₋₆ cycloalkyl, or optionally substituted 4-7 membered heterocyclyl (such as a saturated 4-7 membered heterocyclyl);
   X is N or CR⁵, wherein R⁵ is hydrogen, halogen, CN, CONH₂, COOH, CONH(R^{C}), CON(R^{C})₂, OR^{C}, NO₂, or COOR^{C}, wherein R^{C} at each occurrence is independently an optionally substituted C₁₋₄ alkyl;
   U is N or CR⁶, wherein R⁶ is hydrogen, halogen (e.g., F, Cl, or Br), CN, OH, NH₂, R^{D}, OR^{D}, NH(R^{D}), N(R^{D})₂, COOH, CONH₂, COOR^{D}, CONH(R^{D}), CON(R^{D})₂, SR^{D}, SOR^{D}, SO₂R^{D}, or P(O)(R^{D})₂, wherein R^{D} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
   R⁹ at each occurrence is independently R^{N}, COOR^{N}, COR^{N}, CONH(R^{N}), or CON(R^{N})₂, wherein R^{N} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, or optionally substituted 5 or 6 membered heteroaryl;
   n is 0, 1, 2, 3, or 4;
   L² is absent, C(O), SO₂, an optionally substituted C₁₋₄ alkylene, an optionally substituted C₂₋₄ alkenylene, an optionally substituted C₁₋₄ alkynylene, optionally substituted C₁₋₄ heteroalkylene, or an optionally substituted 3-7 membered ring; and
   R³⁰ is hydrogen, an optionally substituted phenyl, optionally substituted 5 or 6 membered heteroaryl, or optionally substituted 8-12 membered ring having two or more rings.

In some embodiments, the compound of Formula V can have stereoisomer(s). In such embodiments, the compound of Formula V can exist in the form of an individual enantiomer, diastereomer, atropisomer, and/or geometric isomer, as applicable, or a mixture of stereoisomers, including racemic mixtures and mixtures enriched in one or more stereoisomers. In some embodiments, when applicable, the compound of Formula V can exist as a mixture of a pair of enantiomers in any ratio, including a racemic mixture with a ratio of 1:1. In some embodiments, when applicable, the compound of Formula V can exist as an isolated or enriched individual enantiomer substantially free (e.g., with less than 20%, less than 10%, less than 5%, less than 1%, by weight, by HPLC or SFC area, or both, or with a non-detectable amount) of the other enantiomer.

Unless otherwise specified or contrary from context, the applicable variables R¹, R³, U, L², R³⁰, R⁹, n, and X suitable for Formula V, include any of those respective definitions in connection with Formula I (e.g., Formula I-1, I-2, I-3, I-4, I-5, I-6, I-A, I-B, I-C, I-A-1, I-B-1, I-C-1, I-D, I-E, I-L-1, I-L-2, I-L-3, I-L-4, I-L-5, I-L-6, I-L-7, I-L-8, I-L-9, I-L-10, or I-L-11), Formula II-1 to II-7, and Formula III-1 to III-9, as described and preferred herein in any combinations. For example, in some embodiments, U is N. In some embodiments, U is CR⁶, wherein R⁶ is defined herein. In some embodiments, R⁶ is hydrogen. In some embodiments, R⁶ is halogen or OR^{D}1, wherein R^{D}1 is optionally substituted C₁₋₄ alkyl, optionally substituted C₃₋₆ cycloalkyl, or optionally substituted 4-7 membered heterocyclyl having 1 or 2 ring heteroatoms independently selected from O, N, and S; wherein, when substituted, the optionally substituted C₁₋₄ alkyl, C₃₋₆ cycloalkyl, or 4-7 membered heterocyclyl can be substituted with one or more (e.g., 1-3) substituents, for example, each substituent can be independently selected from halogen (e.g., F), OH, CN, C₁₋₄ alkyl or C₁₋₄ alkoxy. In some embodiments, R⁶ is C₁₋₄ alkoxy. In some embodiments, R⁶ is

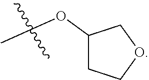 [SMILES: *OC1CCOC1]

In some specific embodiments, R⁶ can be

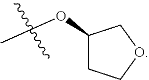 [SMILES: *O[C@@H]1CCOC1]

In some specific embodiments, R⁶ can be

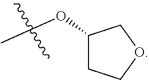 [SMILES: *O[C@H]1CCOC1]

In some embodiments, R¹ is CN. In some embodiments, R¹ can be characterized in that (i) R¹ is halogen, e.g., F or Cl; (ii) R¹ is

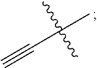 [SMILES: *C#C]

(iii) R¹ is an optionally substituted 5 or 6 membered heteroaryl having 1-4 ring heteroatoms independently selected from O, S, and N, such as an optionally substituted pyrimidinyl or an optionally substituted thiazolyl, for example, R¹ is

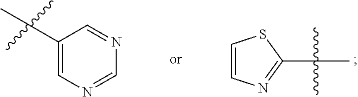 [SMILES: *c1cncnc1 ; *c1nccs1]

or (iv) R¹ is SR^{A1}, wherein R^{A}1 is independently optionally substituted C₁₋₄ alkyl, for example, R¹ is SCH₃. In some embodiments, R³ is an optionally substituted C₁₋₄ alkyl, such as CH₃, CD₃ etc. In some embodiments, X is N. In some embodiments, X is CR⁵, wherein R⁵ is CN. In some embodiments, X is CR⁵, wherein R⁵ is CONH₂, COOH, CONH(R^{C1}), CON(R^{C1})₂, or COOR^{C1}, wherein Rei at each occurrence is independently a C₁₋₄ alkyl, such as methyl. In some embodiments, n is 0, 1, or 2. In some embodiments, R⁹ at each occurrence is independently CH₃, CH₂CH₃, CH₂CH₂CH₃, fluorine-substituted C₁₋₃ alkyl (e.g., CF₂H), cyclopropyl, cyclobutyl, CH₂OH, CH₂OCH₃, CH₂OCH₂CH₃, CH₂NH₂, CH₂N₃, or CH₂NHC(O)OCH₃.

For example, in some embodiments, n is 0. In some embodiments, n is 1 or 2, and the compound of Formula V can be characterized as having a structure according to Formula V-1, V-2, or V-3:

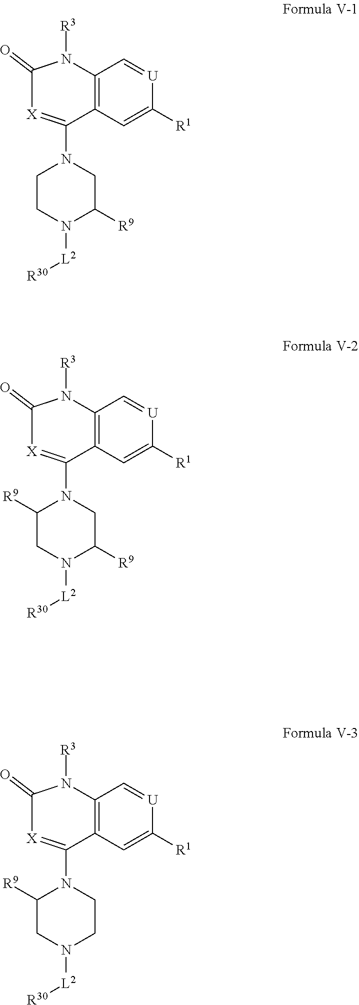

wherein the variables R¹, R³, U, L², R³⁰, R⁹, and X include any of those respective definitions herein.

In some embodiments, in Formula V, L² can be absent.

In some embodiments, L² in Formula V can be an optionally substituted C₁₋₄ alkylene selected from:

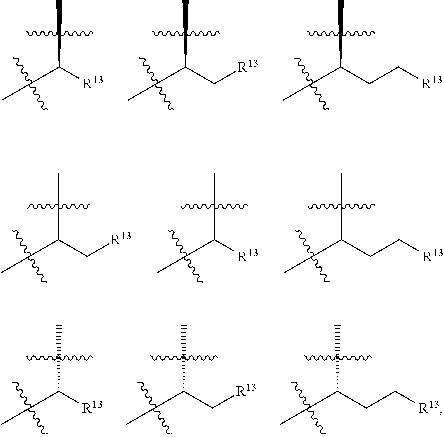

wherein:
R¹³ is selected from hydrogen, halogen (e.g., F, Cl, or Br), CN, OH, NH₂, COOH, CONH₂, SO₂NH₂, COR^{K}, COOR^{K}, CONH(R^{K}), CON(R^{K})₂, SO₂R^{K}, SO₂NH(R^{K}), SO₂N(R^{K})₂, R^{K}, OR^{K}, NH(R^{K}), N(R^{K})₂, SR^{K}, SOR^{K}, SO₂R^{K}, or P(O)(R^{K})₂, wherein R^{K} at each occurrence is independently optionally substituted C₁₋₄ alkyl, optionally substituted C₂₋₄ alkenyl, optionally substituted C₂₋₄ alkynyl, optionally substituted C₁₋₄ heteroalkyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl, wherein, when substituted, the optionally substituted C₁₋₄ alkyl, C₂₋₄ alkenyl, C₂₋₄ alkynyl, C₁₋₄ heteroalkyl, C₃₋₆ cycloalkyl, phenyl, 5 or 6 membered heteroaryl, or 4-7 membered heterocyclyl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. For example, in some embodiments, R¹³ is hydrogen. In some embodiments, R¹³ is CN, OH, COOH, CONH₂, methoxy, ethoxy, cyclopropyl, cyclobutyl, optionally substituted phenyl, or optionally substituted 5 or 6 membered heteroaryl having 1-3 ring heteroatoms independently selected from N, O, and S, wherein, when substituted, the optionally substituted phenyl or 5 or 6 membered heteroaryl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

In some embodiments, in Formula V, L² can be C(O).

In some embodiments, in Formula V, L² can be an optionally substituted 5 or 6 membered heteroarylene, for example,

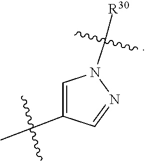

In some embodiments, in Formula V, R³⁰ is optionally substituted phenyl, such as a phenyl which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl. For example, in some embodiments, R³⁰ is a phenyl which is substituted with 1-3 (e.g., 1 or 2) R²², wherein R²² at each occurrence is independently halogen (e.g., F, Cl, or Br), CN, OH, R^{M1}, OR^{M1}, SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. More preferably, R³⁰ is a phenyl which is substituted with 1-3 (e.g., 1 or 2) R²², wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

In some embodiments, in Formula V, R³⁰ is optionally substituted 5 or 6-membered heteroaryl, such as a pyridyl

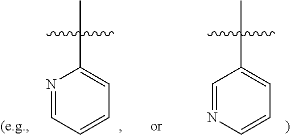 [SMILES: *c1ccccn1 ; *c1cccnc1]

which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from 0, S, and N, or optionally substituted 4-7 membered heterocyclyl. For example, in some embodiments, R³⁰ is a 5-membered heteroaryl

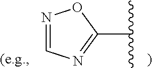 [SMILES: *c1ncno1]

or pyridyl

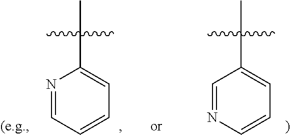 [SMILES: *c1ccccn1 ; *c1cccnc1]

which is substituted with 1-3 (e.g., 1 or 2) R²², as valency permits, wherein R²² at each occurrence is independently halogen (e.g., F, Cl, or Br), CN, OH, R^{M1}, OR^{M1} SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl. More preferably, R³⁰ is a 5-membered heteroaryl

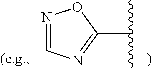 [SMILES: *c1ncno1]

or pyridyl

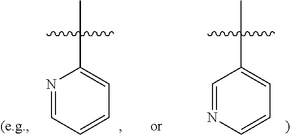 [SMILES: *c1ccccn1 ; *c1cccnc1]

which is substituted with 1-3 (e.g., 1 or 2) R²², as valency permits, wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

In some embodiments, R³⁰ in Formula V can have any of the definitions described for Ring A and R⁴ in connection with Formula I and the subformulae.

For example, in some embodiments, R³⁰ can have a structure according to S-1-A, S-1-B, or S-1-C:

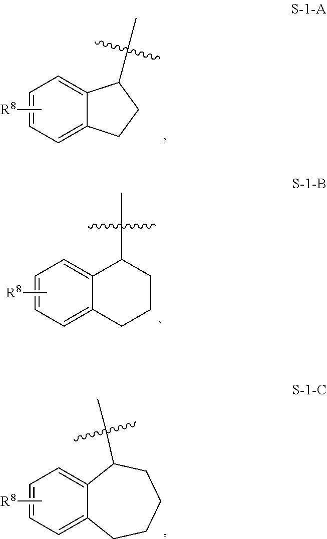

wherein R⁸ is C₁₋₄ alkyl optionally substituted with 1-3 F (such as CH₃, CF₃, etc.), C₁₋₄ alkoxy optionally substituted with 1-3 F (such as OCH₃, OCF₃, etc.), SCF₃, SF₅, cyclopropyl, cyclobutyl, or

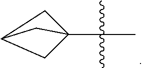 [SMILES: *C12CC(C1)C2]

In some preferred embodiments, R³⁰ in Formula V can be selected from the following:

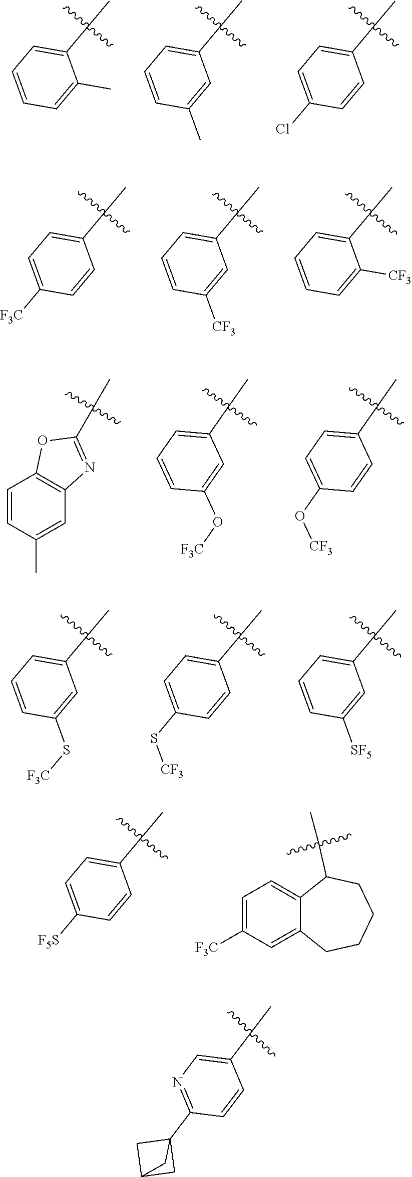

or R³⁰ is selected from:

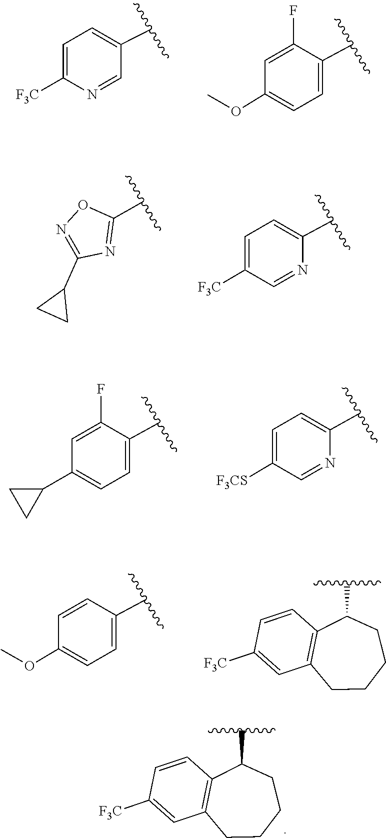

In some embodiments, in Formula V, the piperazine moiety in combination of L² and R³⁰ can together have the L¹-L²-R³⁰ definition as shown for Formula III, such as those shown in enumerated Embodiment A25.

In some embodiments, the present disclosure also provides a compound selected from Compound Nos. 1-134, or a pharmaceutically acceptable salt thereof.

In some embodiments, the present disclosure also provides a compound selected from the compounds shown in Table A below, or a pharmaceutically acceptable salt thereof:

TABLE 1
List of Compounds

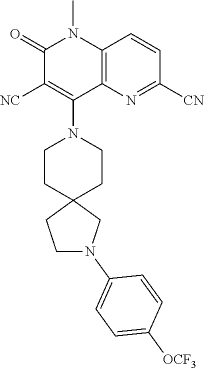 [SMILES: Cn1c(=O)c(C#N)c(N2CCC3(CCN(c4ccc(OC(F)(F)F)cc4)C3)CC2)c2nc(C#N)ccc21]

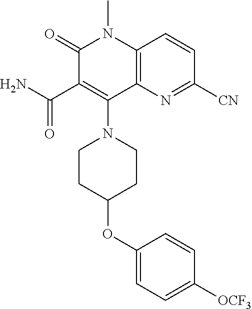 [SMILES: Cn1c(=O)c(C(N)=O)c(N2CCC(Oc3ccc(OC(F)(F)F)cc3)CC2)c2nc(C#N)ccc21]

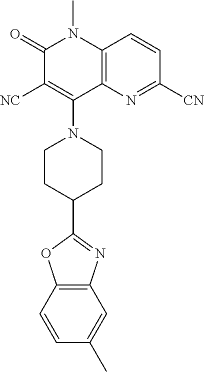 [SMILES: Cc1ccc2oc(C3CCN(c4c(C#N)c(=O)n(C)c5ccc(C#N)nc45)CC3)nc2c1]

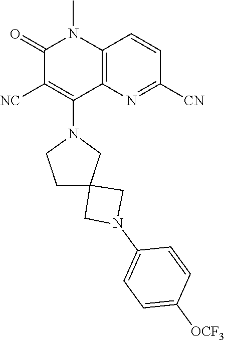 [SMILES: Cn1c(=O)c(C#N)c(N2CCC3(CN(c4ccc(OC(F)(F)F)cc4)C3)C2)c2nc(C#N)ccc21]

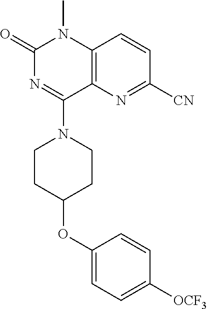 [SMILES: Cn1c(=O)nc(N2CCC(Oc3ccc(OC(F)(F)F)cc3)CC2)c2nc(C#N)ccc21]

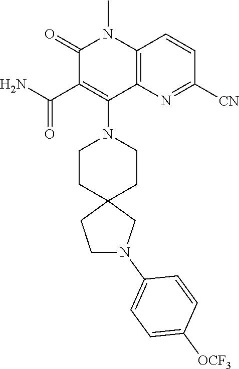 [SMILES: Cn1c(=O)c(C(N)=O)c(N2CCC3(CCN(c4ccc(OC(F)(F)F)cc4)C3)CC2)c2nc(C#N)ccc21]

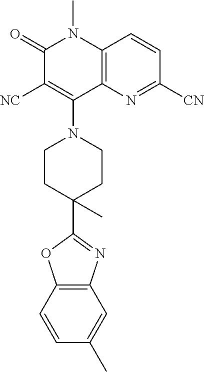 [SMILES: Cc1ccc2oc(C3(C)CCN(c4c(C#N)c(=O)n(C)c5ccc(C#N)nc45)CC3)nc2c1]

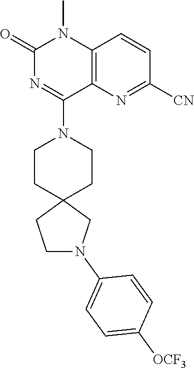 [SMILES: Cn1c(=O)nc(N2CCC3(CCN(c4ccc(OC(F)(F)F)cc4)C3)CC2)c2nc(C#N)ccc21]

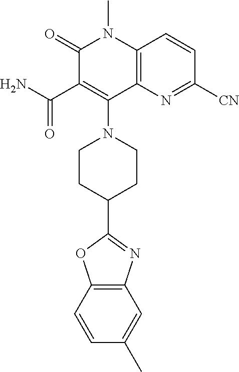 [SMILES: Cc1ccc2oc(C3CCN(c4c(C(N)=O)c(=O)n(C)c5ccc(C#N)nc45)CC3)nc2c1]

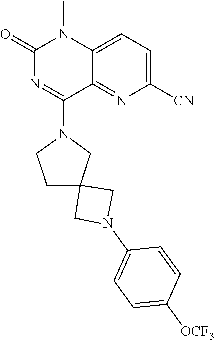 [SMILES: Cn1c(=O)nc(N2CCC3(CN(c4ccc(OC(F)(F)F)cc4)C3)C2)c2nc(C#N)ccc21]

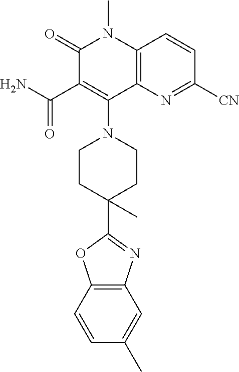 [SMILES: Cc1ccc2oc(C3(C)CCN(c4c(C(N)=O)c(=O)n(C)c5ccc(C#N)nc45)CC3)nc2c1]

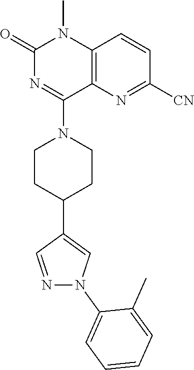 [SMILES: Cc1ccccc1-n1cc(C2CCN(c3nc(=O)n(C)c4ccc(C#N)nc34)CC2)cn1]

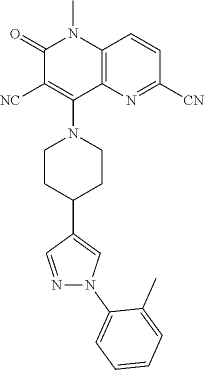 [SMILES: Cc1ccccc1-n1cc(C2CCN(c3c(C#N)c(=O)n(C)c4ccc(C#N)nc34)CC2)cn1]

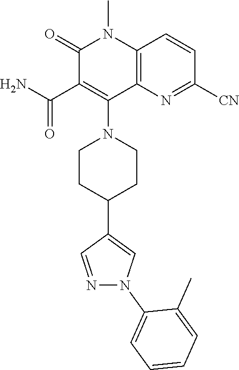 [SMILES: Cc1ccccc1-n1cc(C2CCN(c3c(C(N)=O)c(=O)n(C)c4ccc(C#N)nc34)CC2)cn1]

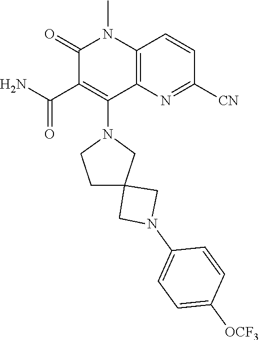 [SMILES: Cn1c(=O)c(C(N)=O)c(N2CCC3(CN(c4ccc(OC(F)(F)F)cc4)C3)C2)c2nc(C#N)ccc21]

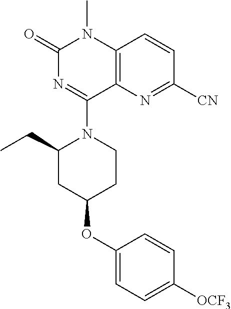 [SMILES: CC[C@@H]1C[C@H](Oc2ccc(OC(F)(F)F)cc2)CCN1c1nc(=O)n(C)c2ccc(C#N)nc12]

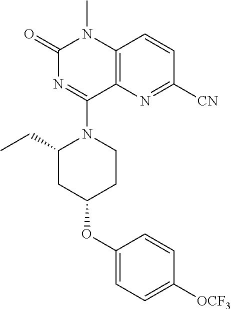 [SMILES: CC[C@H]1C[C@@H](Oc2ccc(OC(F)(F)F)cc2)CCN1c1nc(=O)n(C)c2ccc(C#N)nc12]

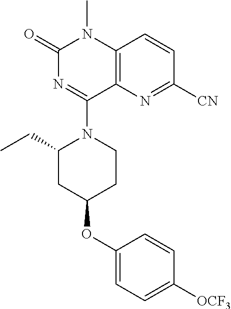 [SMILES: CC[C@H]1C[C@H](Oc2ccc(OC(F)(F)F)cc2)CCN1c1nc(=O)n(C)c2ccc(C#N)nc12]

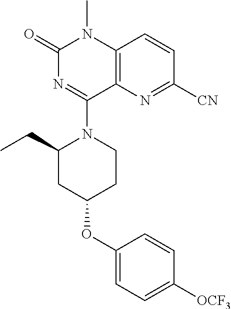 [SMILES: CC[C@@H]1C[C@@H](Oc2ccc(OC(F)(F)F)cc2)CCN1c1nc(=O)n(C)c2ccc(C#N)nc12]

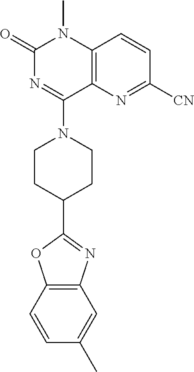 [SMILES: Cc1ccc2oc(C3CCN(c4nc(=O)n(C)c5ccc(C#N)nc45)CC3)nc2c1]

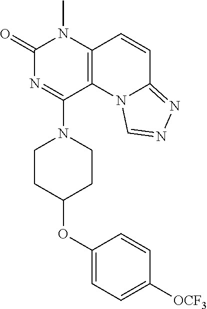 [SMILES: Cn1c(=O)nc(N2CCC(Oc3ccc(OC(F)(F)F)cc3)CC2)c2c1ccc1nncn12]

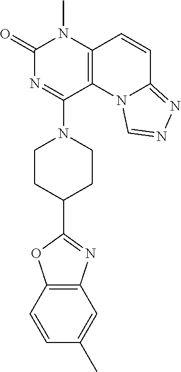 [SMILES: Cc1ccc2oc(C3CCN(c4nc(=O)n(C)c5ccc6nncn6c45)CC3)nc2c1]

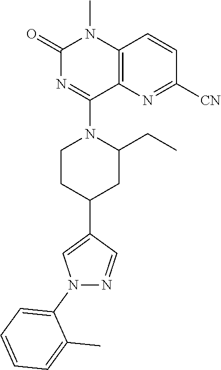 [SMILES: CCC1CC(c2cnn(-c3ccccc3C)c2)CCN1c1nc(=O)n(C)c2ccc(C#N)nc12]

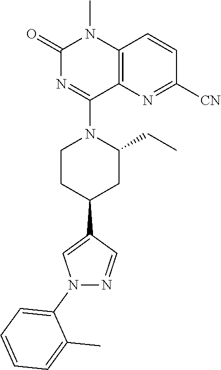 [SMILES: CC[C@@H]1C[C@@H](c2cnn(-c3ccccc3C)c2)CCN1c1nc(=O)n(C)c2ccc(C#N)nc12]

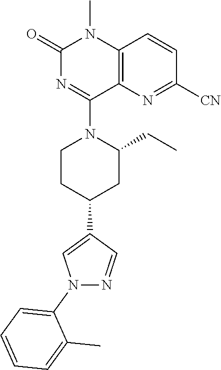 [SMILES: CC[C@@H]1C[C@H](c2cnn(-c3ccccc3C)c2)CCN1c1nc(=O)n(C)c2ccc(C#N)nc12]

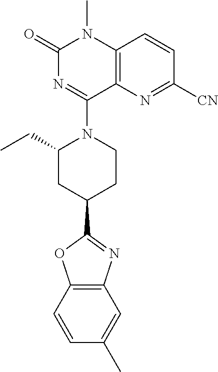 [SMILES: CC[C@H]1C[C@H](c2nc3cc(C)ccc3o2)CCN1c1nc(=O)n(C)c2ccc(C#N)nc12]

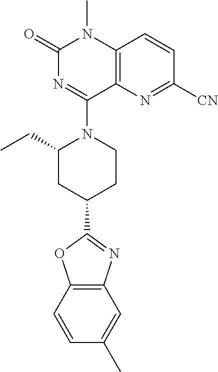 [SMILES: CC[C@H]1C[C@@H](c2nc3cc(C)ccc3o2)CCN1c1nc(=O)n(C)c2ccc(C#N)nc12]

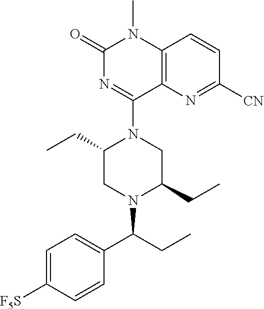

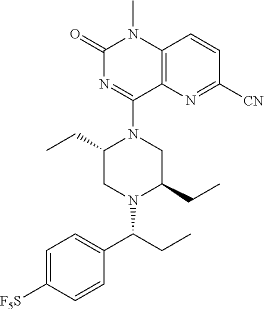

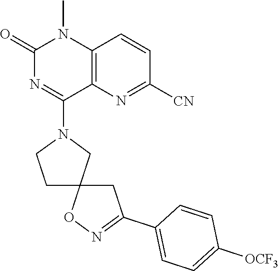 [SMILES: Cn1c(=O)nc(N2CCC3(CC(c4ccc(OC(F)(F)F)cc4)=NO3)C2)c2nc(C#N)ccc21]

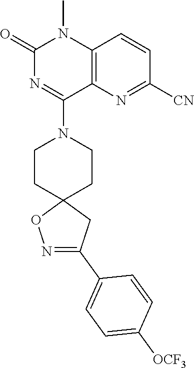 [SMILES: Cn1c(=O)nc(N2CCC3(CC2)CC(c2ccc(OC(F)(F)F)cc2)=NO3)c2nc(C#N)ccc21]

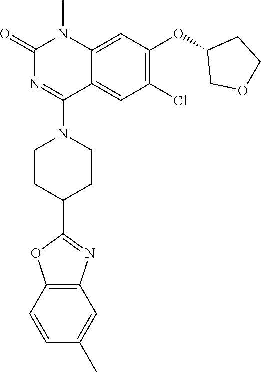 [SMILES: Cc1ccc2oc(C3CCN(c4nc(=O)n(C)c5cc(O[C@@H]6CCOC6)c(Cl)cc45)CC3)nc2c1]

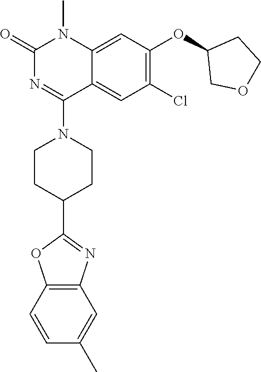 [SMILES: Cc1ccc2oc(C3CCN(c4nc(=O)n(C)c5cc(O[C@H]6CCOC6)c(Cl)cc45)CC3)nc2c1]

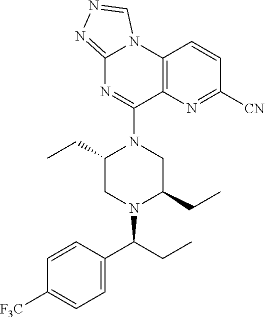 [SMILES: CC[C@H]1CN([C@@H](CC)c2ccc(C(F)(F)F)cc2)[C@H](CC)CN1c1nc2nncn2c2ccc(C#N)nc12]

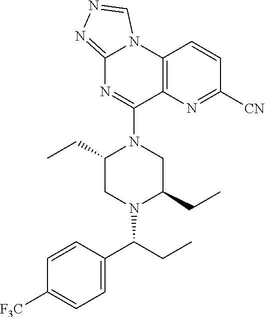 [SMILES: CC[C@H]1CN([C@H](CC)c2ccc(C(F)(F)F)cc2)[C@H](CC)CN1c1nc2nncn2c2ccc(C#N)nc12]

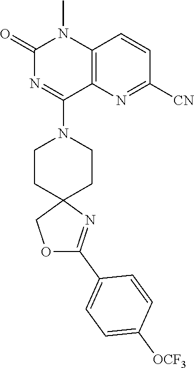 [SMILES: Cn1c(=O)nc(N2CCC3(CC2)COC(c2ccc(OC(F)(F)F)cc2)=N3)c2nc(C#N)ccc21]

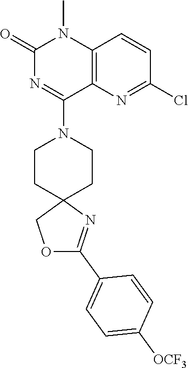 [SMILES: Cn1c(=O)nc(N2CCC3(CC2)COC(c2ccc(OC(F)(F)F)cc2)=N3)c2nc(Cl)ccc21]

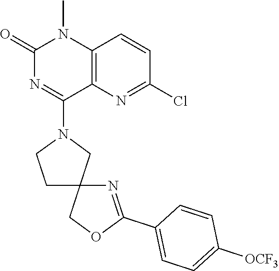 [SMILES: Cn1c(=O)nc(N2CCC3(COC(c4ccc(OC(F)(F)F)cc4)=N3)C2)c2nc(Cl)ccc21]

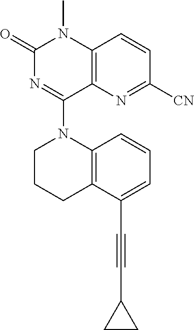 [SMILES: Cn1c(=O)nc(N2CCCc3c(C#CC4CC4)cccc32)c2nc(C#N)ccc21]

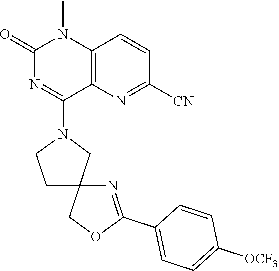 [SMILES: Cn1c(=O)nc(N2CCC3(COC(c4ccc(OC(F)(F)F)cc4)=N3)C2)c2nc(C#N)ccc21]

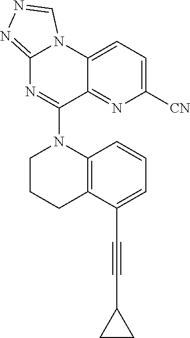 [SMILES: N#Cc1ccc2c(n1)c(N1CCCc3c(C#CC4CC4)cccc31)nc1nncn12]

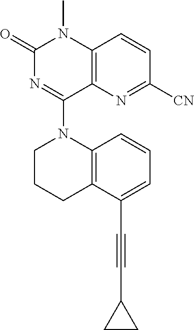 [SMILES: Cn1c(=O)nc(N2CCCc3c(C#CC4CC4)cccc32)c2nc(C#N)ccc21]

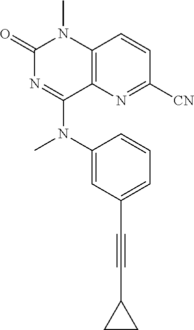 [SMILES: CN(c1cccc(C#CC2CC2)c1)c1nc(=O)n(C)c2ccc(C#N)nc12]

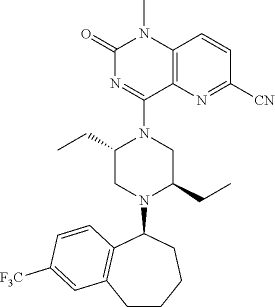 [SMILES: CC[C@H]1CN([C@H]2CCCCc3cc(C(F)(F)F)ccc32)[C@H](CC)CN1c1nc(=O)n(C)c2ccc(C#N)nc12]

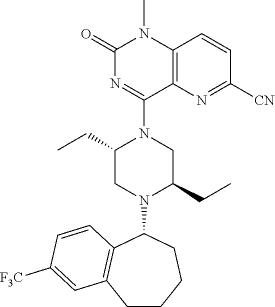 [SMILES: CC[C@H]1CN([C@@H]2CCCCc3cc(C(F)(F)F)ccc32)[C@H](CC)CN1c1nc(=O)n(C)c2ccc(C#N)nc12]

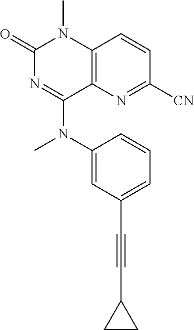 [SMILES: CN(c1cccc(C#CC2CC2)c1)c1nc(=O)n(C)c2ccc(C#N)nc12]

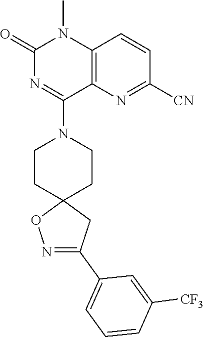 [SMILES: Cn1c(=O)nc(N2CCC3(CC2)CC(c2cccc(C(F)(F)F)c2)=NO3)c2nc(C#N)ccc21]

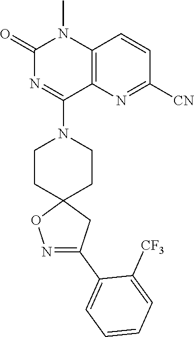 [SMILES: Cn1c(=O)nc(N2CCC3(CC2)CC(c2ccccc2C(F)(F)F)=NO3)c2nc(C#N)ccc21]

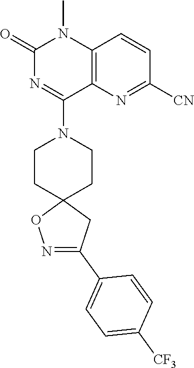 [SMILES: Cn1c(=O)nc(N2CCC3(CC2)CC(c2ccc(C(F)(F)F)cc2)=NO3)c2nc(C#N)ccc21]

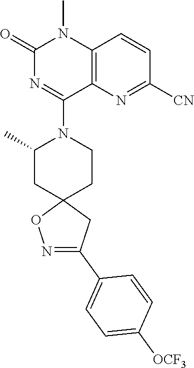 [SMILES: C[C@H]1CC2(CCN1c1nc(=O)n(C)c3ccc(C#N)nc13)CC(c1ccc(OC(F)(F)F)cc1)=NO2]

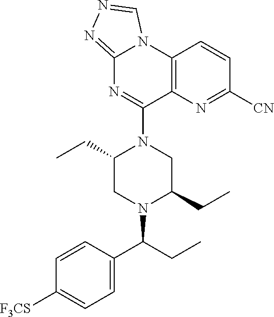

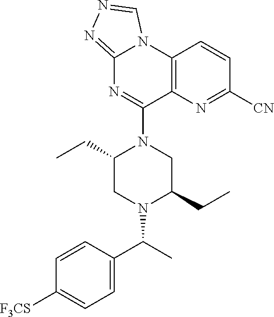

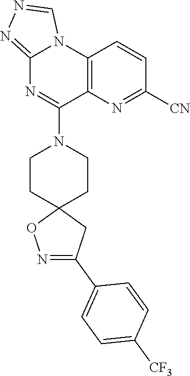 [SMILES: N#Cc1ccc2c(n1)c(N1CCC3(CC1)CC(c1ccc(C(F)(F)F)cc1)=NO3)nc1nncn12]

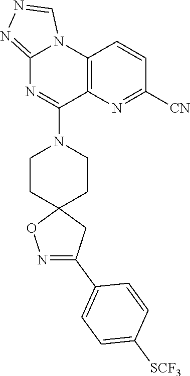

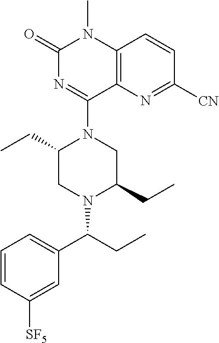

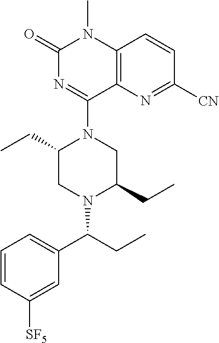

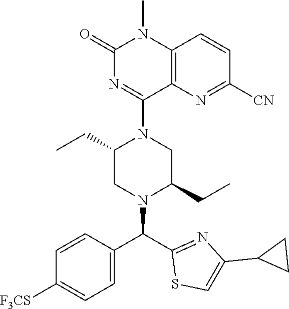

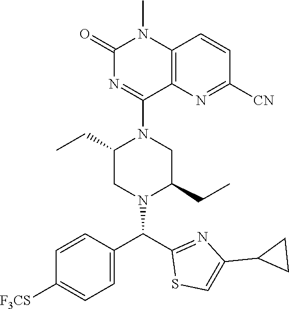

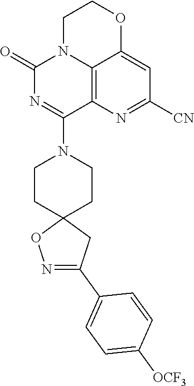 [SMILES: N#Cc1cc2c3c(n1)c(N1CCC4(CC1)CC(c1ccc(OC(F)(F)F)cc1)=NO4)nc(=O)n3CCO2]

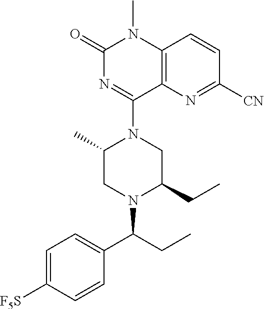

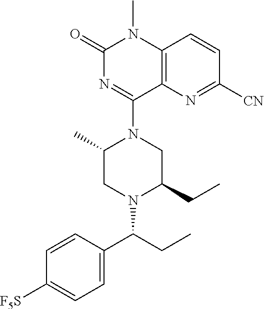

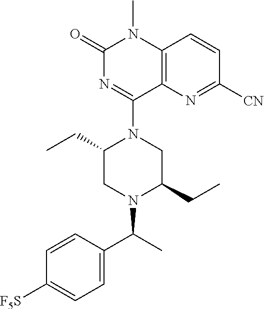

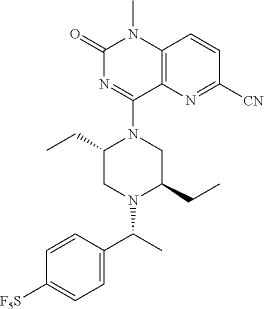

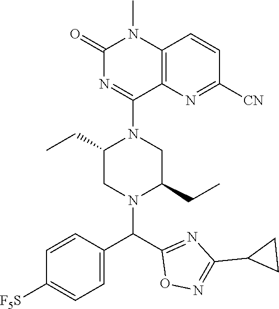

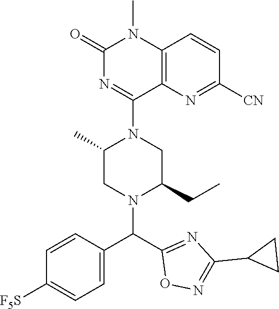

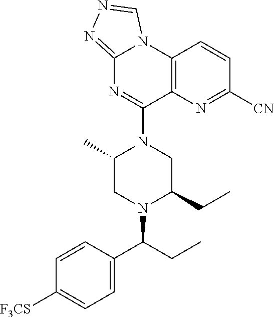

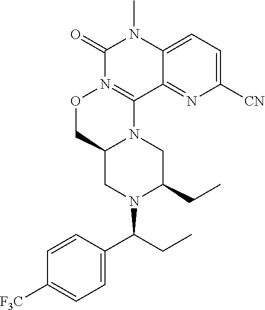

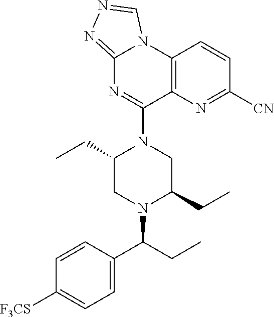

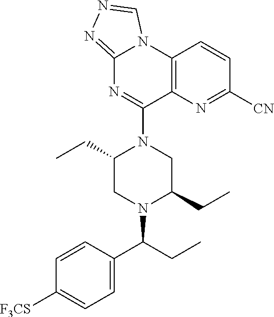

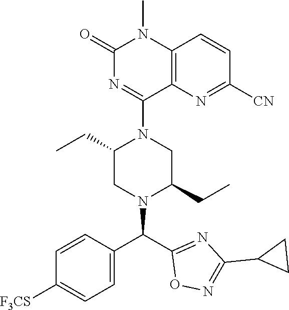

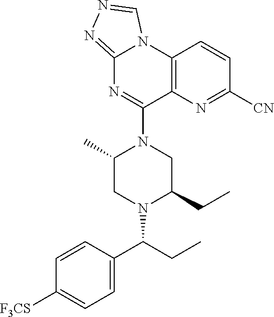

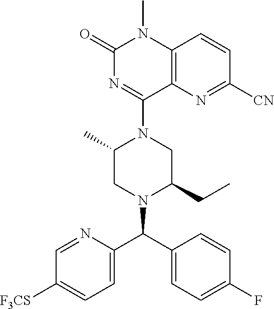

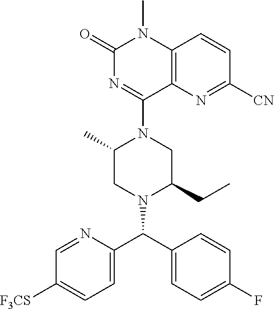

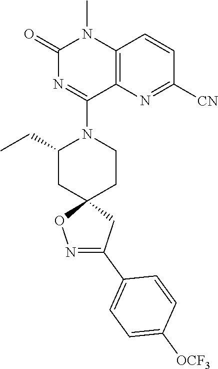 [SMILES: CC[C@H]1C[C@]2(CCN1c1nc(=O)n(C)c3ccc(C#N)nc13)CC(c1ccc(OC(F)(F)F)cc1)=NO2]

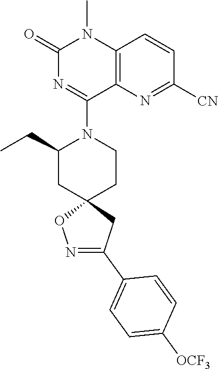 [SMILES: CC[C@@H]1C[C@@]2(CCN1c1nc(=O)n(C)c3ccc(C#N)nc13)CC(c1ccc(OC(F)(F)F)cc1)=NO2]

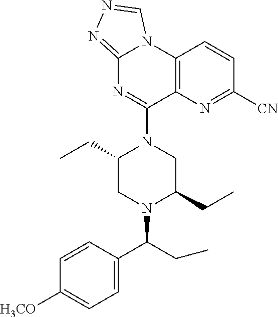 [SMILES: CC[C@H]1CN([C@@H](CC)c2ccc(OC)cc2)[C@H](CC)CN1c1nc2nncn2c2ccc(C#N)nc12]

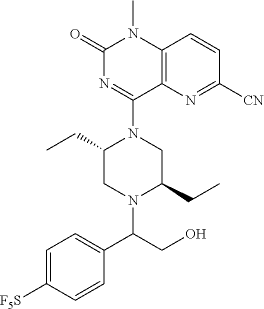

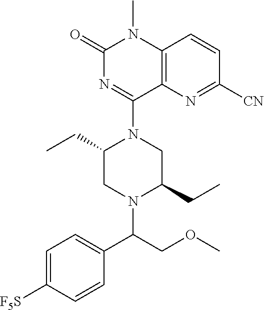

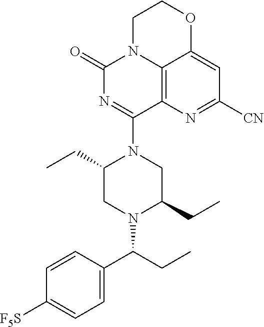

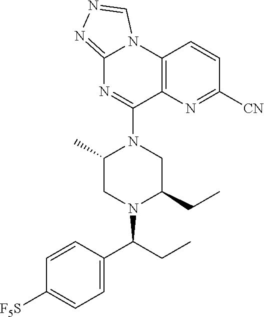

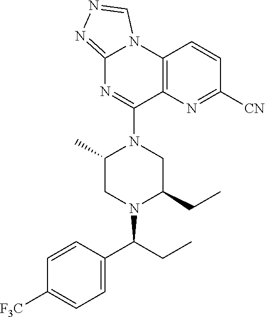 [SMILES: CC[C@@H]1CN(c2nc3nncn3c3ccc(C#N)nc23)[C@@H](C)CN1[C@@H](CC)c1ccc(C(F)(F)F)cc1]

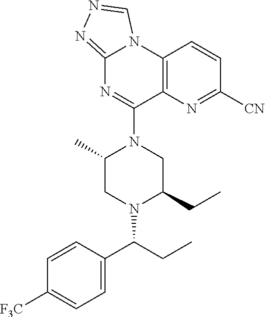 [SMILES: CC[C@@H]1CN(c2nc3nncn3c3ccc(C#N)nc23)[C@@H](C)CN1[C@H](CC)c1ccc(C(F)(F)F)cc1]

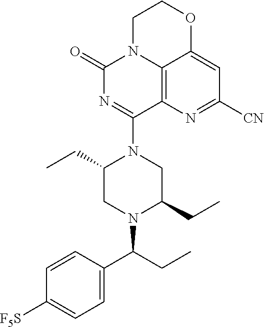

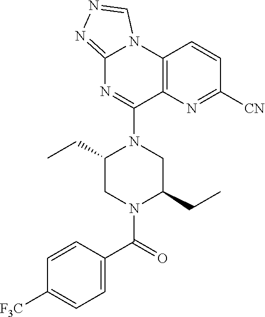 [SMILES: CC[C@@H]1CN(c2nc3nncn3c3ccc(C#N)nc23)[C@@H](CC)CN1C(=O)c1ccc(C(F)(F)F)cc1]

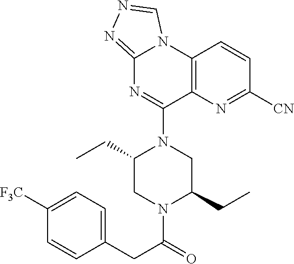 [SMILES: CC[C@@H]1CN(c2nc3nncn3c3ccc(C#N)nc23)[C@@H](CC)CN1C(=O)Cc1ccc(C(F)(F)F)cc1]

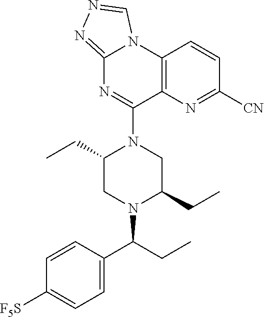

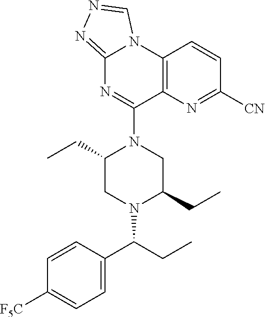

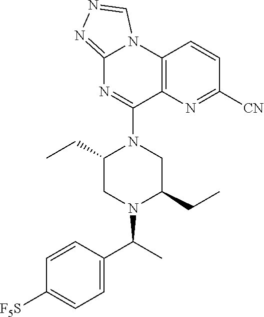

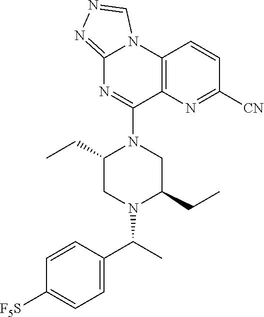

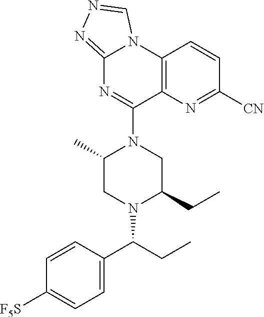

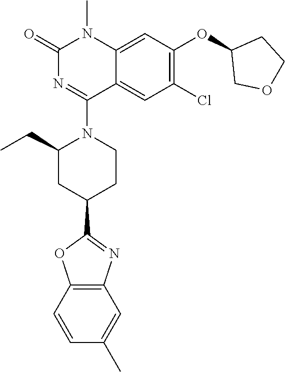 [SMILES: CC[C@@H]1C[C@H](c2nc3cc(C)ccc3o2)CCN1c1nc(=O)n(C)c2cc(O[C@H]3CCOC3)c(Cl)cc12]

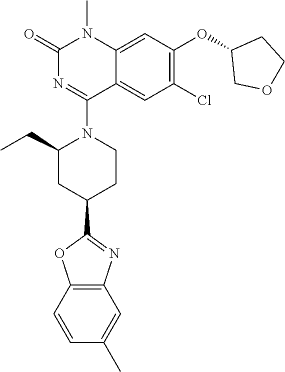 [SMILES: CC[C@@H]1C[C@H](c2nc3cc(C)ccc3o2)CCN1c1nc(=O)n(C)c2cc(O[C@@H]3CCOC3)c(Cl)cc12]

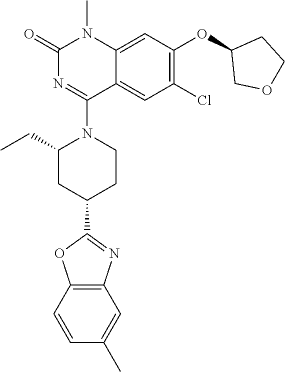 [SMILES: CC[C@H]1C[C@@H](c2nc3cc(C)ccc3o2)CCN1c1nc(=O)n(C)c2cc(O[C@H]3CCOC3)c(Cl)cc12]

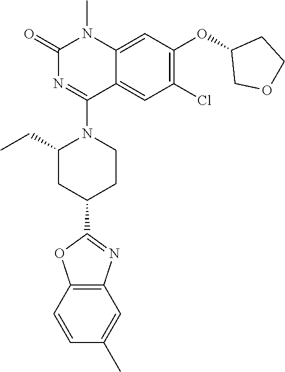 [SMILES: CC[C@H]1C[C@@H](c2nc3cc(C)ccc3o2)CCN1c1nc(=O)n(C)c2cc(O[C@@H]3CCOC3)c(Cl)cc12]

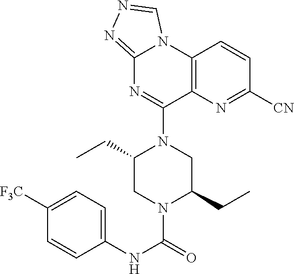 [SMILES: CC[C@@H]1CN(c2nc3nncn3c3ccc(C#N)nc23)[C@@H](CC)CN1C(=O)Nc1ccc(C(F)(F)F)cc1]

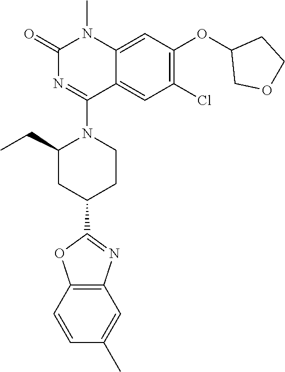 [SMILES: CC[C@@H]1C[C@@H](c2nc3cc(C)ccc3o2)CCN1c1nc(=O)n(C)c2cc(OC3CCOC3)c(Cl)cc12]

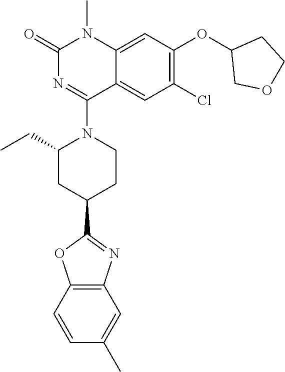 [SMILES: CC[C@H]1C[C@H](c2nc3cc(C)ccc3o2)CCN1c1nc(=O)n(C)c2cc(OC3CCOC3)c(Cl)cc12]

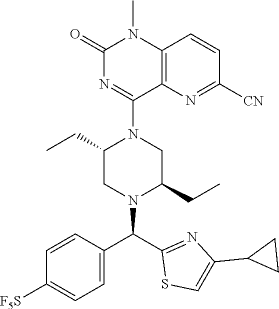

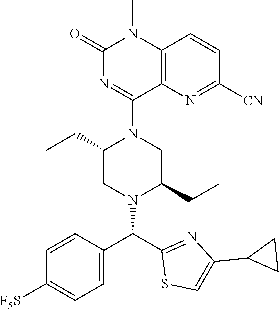

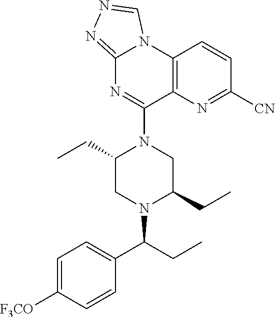 [SMILES: CC[C@H]1CN([C@@H](CC)c2ccc(OC(F)(F)F)cc2)[C@H](CC)CN1c1nc2nncn2c2ccc(C#N)nc12]

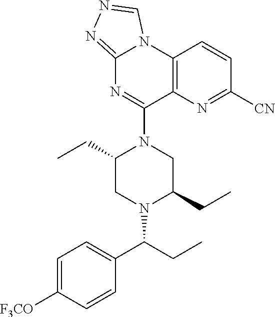 [SMILES: CC[C@H]1CN([C@H](CC)c2ccc(OC(F)(F)F)cc2)[C@H](CC)CN1c1nc2nncn2c2ccc(C#N)nc12]

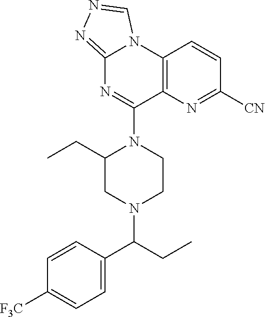 [SMILES: CCC(c1ccc(C(F)(F)F)cc1)N1CCN(c2nc3nncn3c3ccc(C#N)nc23)C(CC)C1]

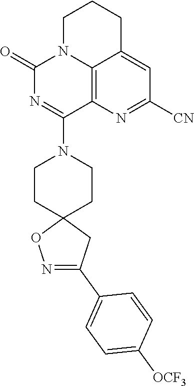 [SMILES: N#Cc1cc2c3c(n1)c(N1CCC4(CC1)CC(c1ccc(OC(F)(F)F)cc1)=NO4)nc(=O)n3CCC2]

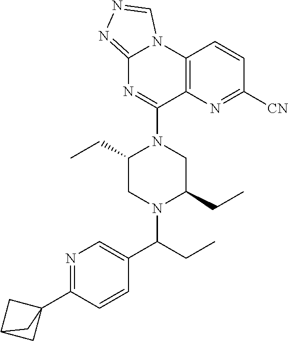 [SMILES: CCC(c1ccc(C23CC(C2)C3)nc1)N1C[C@H](CC)N(c2nc3nncn3c3ccc(C#N)nc23)C[C@H]1CC]

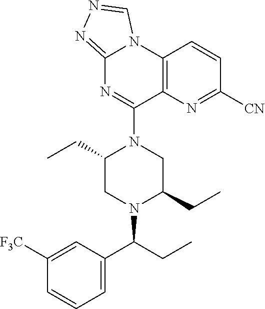 [SMILES: CC[C@H]1CN([C@@H](CC)c2cccc(C(F)(F)F)c2)[C@H](CC)CN1c1nc2nncn2c2ccc(C#N)nc12]

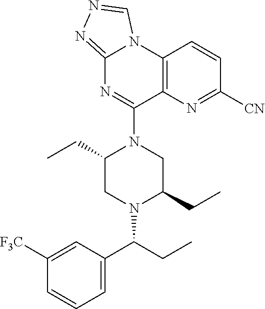 [SMILES: CC[C@H]1CN([C@H](CC)c2cccc(C(F)(F)F)c2)[C@H](CC)CN1c1nc2nncn2c2ccc(C#N)nc12]

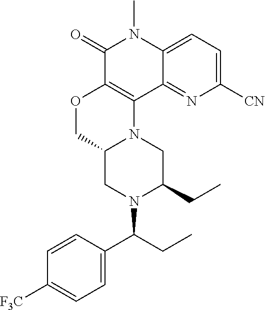 [SMILES: CC[C@@H]1CN2c3c(c(=O)n(C)c4ccc(C#N)nc34)OC[C@H]2CN1[C@@H](CC)c1ccc(C(F)(F)F)cc1]

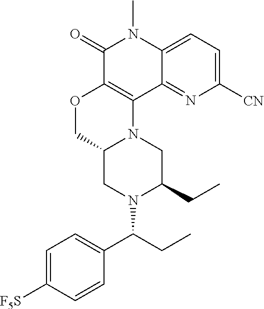

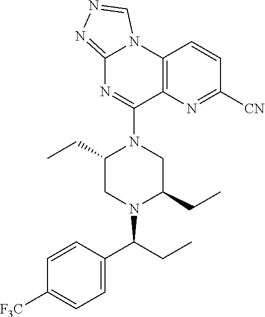 [SMILES: CC[C@H]1CN([C@@H](CC)c2ccc(C(F)(F)F)cc2)[C@H](CC)CN1c1nc2nncn2c2ccc(C#N)nc12]

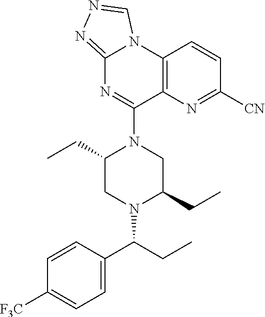 [SMILES: CC[C@H]1CN([C@H](CC)c2ccc(C(F)(F)F)cc2)[C@H](CC)CN1c1nc2nncn2c2ccc(C#N)nc12]

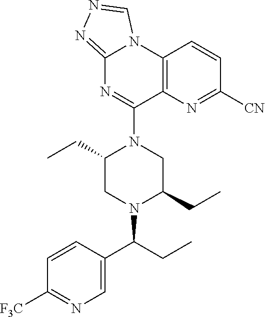 [SMILES: CC[C@H]1CN([C@@H](CC)c2ccc(C(F)(F)F)nc2)[C@H](CC)CN1c1nc2nncn2c2ccc(C#N)nc12]

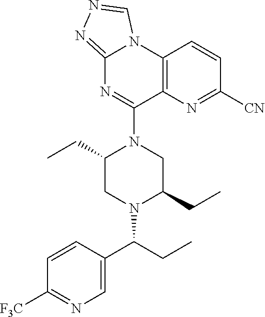 [SMILES: CC[C@H]1CN([C@H](CC)c2ccc(C(F)(F)F)nc2)[C@H](CC)CN1c1nc2nncn2c2ccc(C#N)nc12]

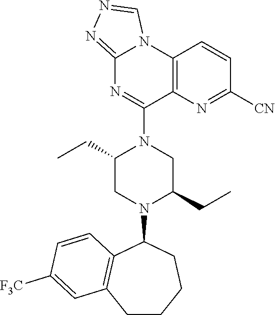 [SMILES: CC[C@H]1CN([C@H]2CCCCc3cc(C(F)(F)F)ccc32)[C@H](CC)CN1c1nc2nncn2c2ccc(C#N)nc12]

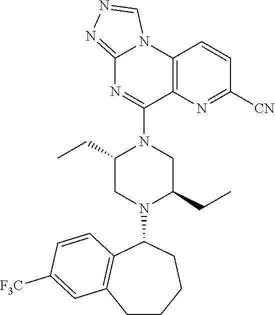 [SMILES: CC[C@H]1CN([C@@H]2CCCCc3cc(C(F)(F)F)ccc32)[C@H](CC)CN1c1nc2nncn2c2ccc(C#N)nc12]

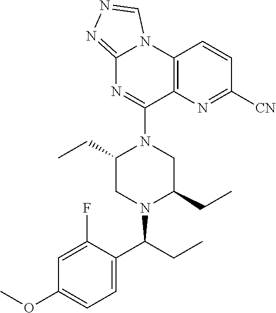 [SMILES: CC[C@H]1CN([C@@H](CC)c2ccc(OC)cc2F)[C@H](CC)CN1c1nc2nncn2c2ccc(C#N)nc12]

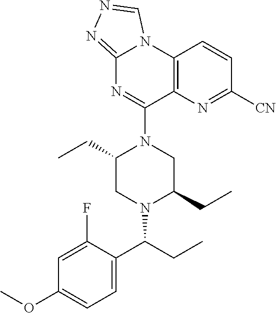 [SMILES: CC[C@H]1CN([C@H](CC)c2ccc(OC)cc2F)[C@H](CC)CN1c1nc2nncn2c2ccc(C#N)nc12]

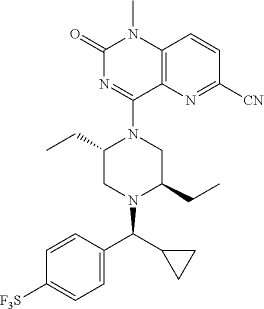

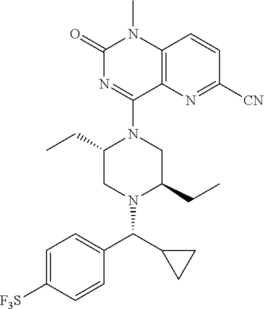

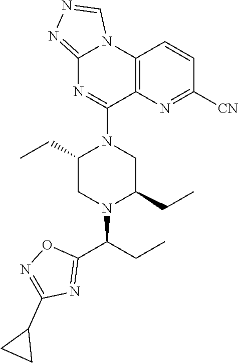 [SMILES: CC[C@H]1CN([C@@H](CC)c2nc(C3CC3)no2)[C@H](CC)CN1c1nc2nncn2c2ccc(C#N)nc12]

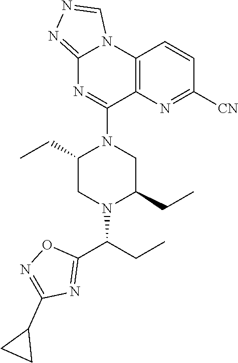 [SMILES: CC[C@H]1CN([C@H](CC)c2nc(C3CC3)no2)[C@H](CC)CN1c1nc2nncn2c2ccc(C#N)nc12]

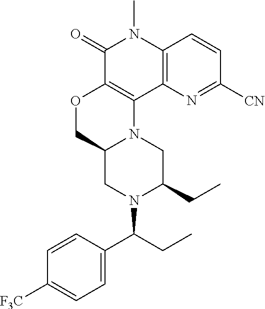 [SMILES: CC[C@@H]1CN2c3c(c(=O)n(C)c4ccc(C#N)nc34)OC[C@@H]2CN1[C@@H](CC)c1ccc(C(F)(F)F)cc1]

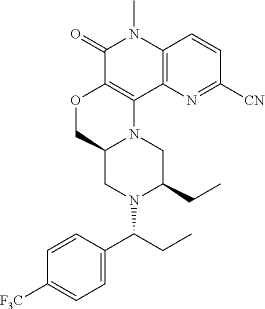 [SMILES: CC[C@@H]1CN2c3c(c(=O)n(C)c4ccc(C#N)nc34)OC[C@@H]2CN1[C@H](CC)c1ccc(C(F)(F)F)cc1]

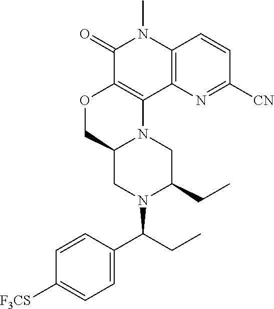

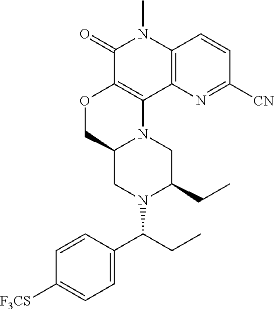

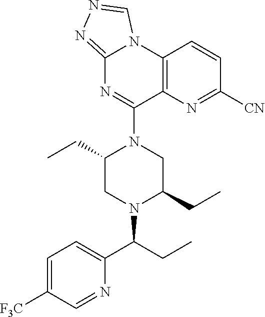 [SMILES: CC[C@H]1CN([C@@H](CC)c2ccc(C(F)(F)F)cn2)[C@H](CC)CN1c1nc2nncn2c2ccc(C#N)nc12]

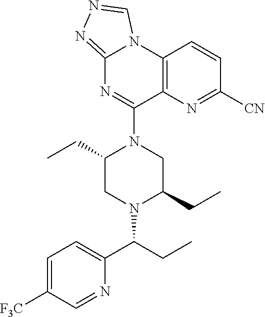 [SMILES: CC[C@H]1CN([C@H](CC)c2ccc(C(F)(F)F)cn2)[C@H](CC)CN1c1nc2nncn2c2ccc(C#N)nc12]

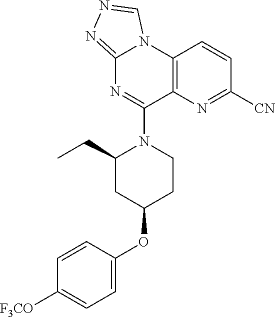 [SMILES: CC[C@@H]1C[C@H](Oc2ccc(OC(F)(F)F)cc2)CCN1c1nc2nncn2c2ccc(C#N)nc12]

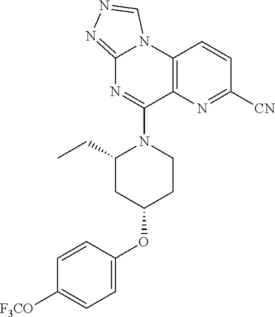 [SMILES: CC[C@H]1C[C@@H](Oc2ccc(OC(F)(F)F)cc2)CCN1c1nc2nncn2c2ccc(C#N)nc12]

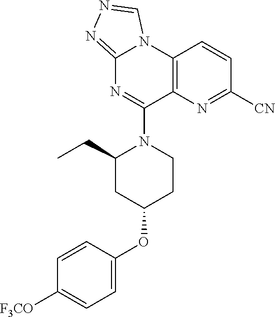 [SMILES: CC[C@@H]1C[C@@H](Oc2ccc(OC(F)(F)F)cc2)CCN1c1nc2nncn2c2ccc(C#N)nc12]

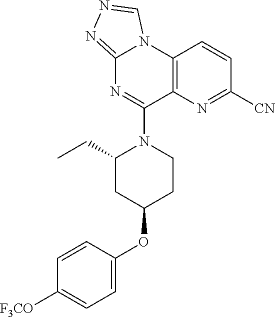 [SMILES: CC[C@H]1C[C@H](Oc2ccc(OC(F)(F)F)cc2)CCN1c1nc2nncn2c2ccc(C#N)nc12]

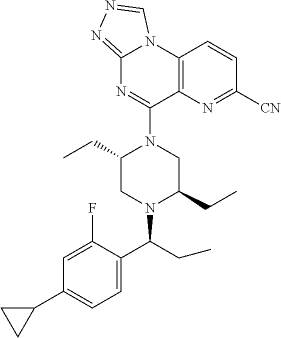 [SMILES: CC[C@H]1CN([C@@H](CC)c2ccc(C3CC3)cc2F)[C@H](CC)CN1c1nc2nncn2c2ccc(C#N)nc12]

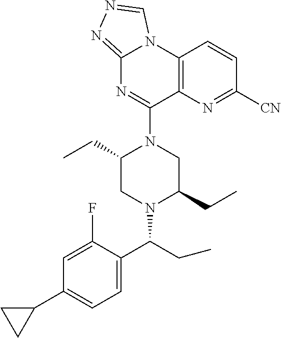 [SMILES: CC[C@H]1CN([C@H](CC)c2ccc(C3CC3)cc2F)[C@H](CC)CN1c1nc2nncn2c2ccc(C#N)nc12]

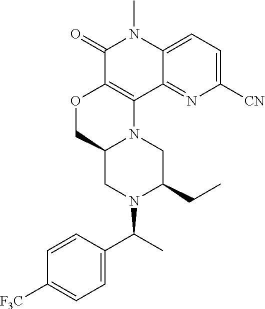 [SMILES: CC[C@@H]1CN2c3c(c(=O)n(C)c4ccc(C#N)nc34)OC[C@@H]2CN1[C@@H](C)c1ccc(C(F)(F)F)cc1]

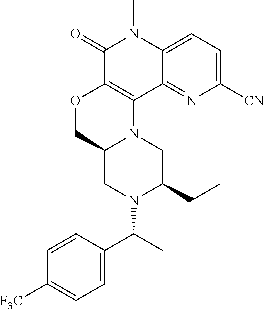 [SMILES: CC[C@@H]1CN2c3c(c(=O)n(C)c4ccc(C#N)nc34)OC[C@@H]2CN1[C@H](C)c1ccc(C(F)(F)F)cc1]

Exemplary synthesis and characterization of the above compounds are shown in the Examples section. The compounds may be prepared in a racemic form, with respect to one or more of the chiral centers, which can be separated into two enantiomers, including the as-drawn enantiomer, or be prepared through chiral synthesis, in view of the present disclosure.

In some embodiments, to the extent applicable, the genus of compounds in the present disclosure also excludes any of the compounds specifically prepared and disclosed prior to this disclosure, such as those specific compounds described in WO2019005883; WO2020006016; WO2020006018; WO2021041588; WO2021105115; WO2021105116; WO2021105117; WO2021127554; WO2021130638; WO2021132422; WO2021133748; WO2021133749; WO2021133750; WO2021133751; WO2021133752; WO2021214019; or WO2021214020.

The compounds of the present disclosure can be readily synthesized by one of ordinary skilled in the art in view of the present disclosure. Exemplified syntheses are also shown in the Examples section.

As will be apparent to those having ordinary skill in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4^{th} ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wisconsin, USA), Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wiley-VCH, 1999), and any of available updates as of this filing.

### Pharmaceutical Compositions

Certain embodiments are directed to a pharmaceutical composition comprising one or more of the compounds of the present disclosure.

The pharmaceutical composition can optionally contain a pharmaceutically acceptable excipient. In some embodiments, the pharmaceutical composition comprises a compound of the present disclosure (e.g., a compound of Formula I (e.g., Formula I-1, I-2, I-3, I-4, I-5, I-6, I-A, I-B, I-C, I-A-1, I-B-1, I-C-1, I-D, I-D-1, I-D-2, I-E, I-L-1, I-L-2, I-L-3, I-L-4, I-L-5, I-L-6, I-L-7, I-L-8, I-L-9, I-L-10, or I-L-11), II-1, II-2, II-3 (e.g., II-3-A, II-3-B, or II-3-C), II-4, II-5, II-6, II-7, III-1, III-2 (e.g., III-2-A, III-2-B, III-2-C, III-2-C-1, III-2-C-2, III-2-C-3, III-2-C-4, III-2-D, III-2-D-1, III-2-D-2, III-2-D-3, III-2-D-4, III-2-E-1, III-2-E-2, III-2-E-3, III-2-E-1a, III-2-E-2a, III-2-E-3a, III-2-E-1b, III-2-E-2b, or III-2-E-3b), III-3 (e.g., III-3-A), III-4 (e.g., III-4-A, III-4-B, III-4-C, III-4-D, III-4-C-1, III-4-C-2, III-4-C-3, III-4-C-4, III-4-D-1, or III-4-D-2), III-5, III-6, III-7, III-8 (e.g., III-8-A), III-9, IV, or V (e.g., V-1, V-2, or V-3), any of Compound Nos. 1-134, any compound selected from the compounds shown in Table A herein, or a pharmaceutically acceptable salt thereof) and a pharmaceutically acceptable excipient. Pharmaceutically acceptable excipients are known in the art. Non-limiting suitable excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I (e.g., Formula I-1, I-2, I-3, I-4, I-5, I-6, I-A, I-B, I-C, I-A-1, I-B-1, I-C-1, I-D, I-D-1, I-D-2, I-E, I-L-1, I-L-2, I-L-3, I-L-4, I-L-5, I-L-6, I-L-7, I-L-8, I-L-9, I-L-10, or I-L-11), II-1, II-2, II-3 (e.g., II-3-A, II-3-B, or II-3-C) II-4. II-5. II-6. II-7. III-1. III-2 (e.g., III-2-A, III-2-B, III-2-C, III-2-C-1, III-2-C-2, III-2-C-3, III-2-C-4, III-2-D, III-2-D-1, III-2-D-2, III-2-D-3, III-2-D-4, III-2-E-1, III-2-E-2, III-2-E-3, III-2-E-1a, III-2-E-2a, III-2-E-3a, III-2-E-1b, III-2-E-2b, or III-2-E-3b), III-3 (e.g., III-3-A), III-4 (e.g., III-4-A, III-4-B, III-4-C, III-4-D, III-4-C-1, III-4-C-2, III-4-C-3, III-4-C-4, III-4-D-1, or III-4-D-2), III-5, III-6, III-7, III-8 (e.g., III-8-A), III-9, IV, or V (e.g., V-1, V-2, or V-3), any of Compound Nos. 1-134, any compound selected from the compounds shown in Table A herein, or a pharmaceutically acceptable salt thereof, e.g., in a therapeutically effective amount. In any of the embodiments described herein, the pharmaceutical composition can comprise a therapeutically effective amount of a compound selected from compound Nos. 1-134, or a pharmaceutically acceptable salt thereof. In any of the embodiments described herein, the pharmaceutical composition can comprise a therapeutically effective amount of a compound selected from the compounds shown in Table A herein, or a pharmaceutically acceptable salt thereof.

The pharmaceutical composition can also be formulated for delivery via any of the known routes of delivery, which include but are not limited to oral, parenteral, inhalation, etc.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion. Excipients for the preparation of parenteral formulations are known in the art. Non-limiting suitable excipients include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof.

In some embodiments, the pharmaceutical composition is formulated for inhalation. The inhalable formulations can be, for example, formulated as a nasal spray, dry powder, or an aerosol administrable through a metered-dose inhaler. Excipients for preparing formulations for inhalation are known in the art. Non-limiting suitable excipients include, for example, lactose, talc, silicic acid, aluminum hydroxide, calcium silicates and polyamide powder, and mixtures of these substances. Sprays can additionally contain propellants, such as chlorofluorohydrocarbons and volatile unsubstituted hydrocarbons, such as butane and propane.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure (e.g., a compound of Formula I (e.g., Formula I-1, I-2, I-3, I-4, I-5, I-6, I-A, I-B, I-C, I-A-1, I-B-1, I-C-1, I-D, I-D-1, I-D-2, I-E, I-L-1, I-L-2, I-L-3, I-L-4, I-L-5, I-L-6, I-L-7, I-L-8, I-L-9, I-L-10, or I-L-11), II-1, II-2, II-3 (e.g., II-3-A, II-3-B, or II-3-C), II-4, II-5, II-6, II-7, III-1, III-2 (e.g., III-2-A, III-2-B, III-2-C, III-2-C-1, III-2-C-2, III-2-C-3, III-2-C-4, III-2-D, III-2-D-1, III-2-D-2, III-2-D-3, III-2-D-4, III-2-E-1, III-2-E-2, III-2-E-3, III-2-E-1a, III-2-E-2a, III-2-E-3a, III-2-E-1b, III-2-E-2b, or III-2-E-3b), III-3 (e.g., III-3-A), III-4 (e.g., III-4-A, III-4-B, III-4-C, III-4-D, III-4-C-1, III-4-C-2, III-4-C-3, III-4-C-4, III-4-D-1, or III-4-D-2), III-5, III-6, III-7, III-8 (e.g., III-8-A), III-9, IV, or V (e.g., V-1, V-2, or V-3), any of Compound Nos. 1-134, any compound selected from the compounds shown in Table A herein, or a pharmaceutically acceptable salt thereof). In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disease or disorder as described herein, which can depend on the recipient of the treatment, the disease or disorder being treated and the severity thereof, the composition containing the compound, the time of administration, the route of administration, the duration of treatment, the compound potency (e.g., for inhibiting DGKa and/or DGKz), its rate of clearance and whether or not another drug is co-administered.

For veterinary use, a compound of the present disclosure can be administered as a suitably acceptable formulation in accordance with normal veterinary practice. The veterinarian can readily determine the dosing regimen and route of administration that is most appropriate for a particular animal.

In some embodiments, all the necessary components for the treatment of DGKa and/or DGKz associated diseases or disorders using a compound of the present disclosure either alone or in combination with another agent or intervention traditionally used for the treatment of such disease can be packaged into a kit. Specifically, in some embodiments, the present invention provides a kit for use in the therapeutic intervention of the disease comprising a packaged set of medicaments that include the compound disclosed herein as well as buffers and other components for preparing deliverable forms of said medicaments, and/or devices for delivering such medicaments, and/or any agents that are used in combination therapy with the compound of the present disclosure, and/or instructions for the treatment of the disease packaged with the medicaments. The instructions may be fixed in any tangible medium, such as printed paper, or a computer readable magnetic or optical medium, or instructions to reference a remote computer data source such as a world wide web page accessible via the internet.

### Method of Treatment

Compounds of the present disclosure are useful as therapeutic active substances for the treatment and/or prophylaxis of diseases or disorders that are associated with the activity of DGKa, DGKz, or both DGKa and DGKz, such as DGK target inhibition in T cells. Such diseases or disorders include viral and other infections (e.g., skin infections, GI infection, urinary tract infections, genito-urinary infections, systemic infections), and proliferative diseases (e.g., cancer).

In some embodiments, the present disclosure provides a method of inhibiting the activity of diacylglycerol kinase alpha and/or zeta (DGKa/z) in a cell comprising contacting a cell with an effective amount of one or more compounds of the present disclosure (e.g., a compound of Formula I (e.g., Formula I-1, I-2, I-3, I-4, I-5, I-6, I-A, I-B, I-C, I-A-1, I-B-1, I-C-1, I-D, I-D-1, I-D-2, I-E, I-L-1, I-L-2, I-L-3, I-L-4, I-L-5, I-L-6, I-L-7, I-L-8, I-L-9, I-L-10, or I-L-11), II-1, II-2, II-3 (e.g., II-3-A, II-3-B, or II-3-C), II-4, II-5, II-6, II-7, III-1, III-2 (e.g., III-2-A, III-2-B, III-2-C, III-2-C-1, III-2-C-2, III-2-C-3, III-2-C-4, III-2-D, III-2-D-1, III-2-D-2, III-2-D-3, III-2-D-4, III-2-E-1, III-2-E-2, III-2-E-3, III-2-E-1a, III-2-E-2a, III-2-E-3a, III-2-E-1b, III-2-E-2b, or III-2-E-3b), III-3 (e.g., III-3-A), III-4 (e.g., III-4-A, III-4-B, III-4-C, III-4-D, III-4-C-1, III-4-C-2, III-4-C-3, III-4-C-4, III-4-D-1, or III-4-D-2), III-5, III-6, III-7, III-8 (e.g., III-8-A), III-9, IV, or V (e.g., V-1, V-2, or V-3), any of Compound Nos. 1-134, any compound selected from the compounds shown in Table A herein, or a pharmaceutically acceptable salt thereof). As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal. As used herein, the term “contacting” refers to the bringing together of indicated moieties in an in vitro system or an in vivo system. For example, “contacting” the DGKa and DGKz enzyme with a compound of the present disclosure includes the administration of a compound of the present disclosure to a subject, such as a human, having DGKa and DGKz, as well as, for example, introducing a compound of the present disclosure into a sample containing a cellular or purified preparation containing DGKa and DGKz enzyme. The term “DGK inhibitor” such as a DGKa and/or DGKz inhibitor refers to an agent capable of inhibiting the activity of diacylglycerol kinase alpha and/or diacylglycerol kinase zeta (DGKa and/or DGKz), such as in T cells resulting in T cell stimulation.

In some embodiments, the present disclosure provides a method of treating a disease associated with activity or expression, including abnormal activity and/or overexpression, of DGKa and/or DGKz in a subject in need thereof, the method comprising administering to the subject an effective amount of one or more compounds of the present disclosure (e.g., a compound of Formula I (e.g., Formula I-1, I-2, I-3, I-4, I-5, 1-6, I-A, I-B, I-C, I-A-1, I-B-1, I-C-1, I-D, I-D-1, I-D-2, I-E, I-L-1, I-L-2, I-L-3, I-L-4, I-L-5, I-L-6, I-L-7, I-L-8, I-L-9, I-L-10, or I-L-11), II-1, II-2, II-3 (e.g., II-3-A, II-3-B, or II-3-C), II-4, II-5, II-6, II-7, III-1, III-2 (e.g., III-2-A, III-2-B, III-2-C, III-2-C-1, III-2-C-2, III-2-C-3, III-2-C-4, III-2-D, III-2-D-1, III-2-D-2, III-2-D-3, III-2-D-4, III-2-E-1, III-2-E-2, III-2-E-3, III-2-E-1a, III-2-E-2a, III-2-E-3a, III-2-E-1b, III-2-E-2b, or III-2-E-3b), III-3 (e.g., III-3-A), III-4 (e.g., III-4-A, III-4-B, III-4-C, III-4-D, III-4-C-1, III-4-C-2, III-4-C-3, III-4-C-4, III-4-D-1, or III-4-D-2), III-5, III-6, III-7, III-8 (e.g., III-8-A), III-9, IV, or V (e.g., V-1, V-2, or V-3), any of Compound Nos. 1-134, any compound selected from the compounds shown in Table A herein, or a pharmaceutically acceptable salt thereof). Examples of diseases can include any disease, disorder or condition that is directly or indirectly linked to expression or activity of DGKa and/or DGKz enzyme, such as over expression or abnormal activity. A DGKa and/or DGKz-associated disease can also include any disease, disorder or condition that can be prevented, ameliorated, or cured by modulating DGKa and/or DGKz enzyme activity. Examples of DGKa and/or DGKz associated diseases include cancer and viral infections such as HIV infection, hepatitis B, and hepatitis C. Examples of cancer include cancer of the colon, pancreatic cancer, breast cancer, prostate cancer, lung cancer, ovarian cancer, cervical cancer, renal cancer, cancer of the head and neck, lymphoma, leukemia, and/or melanoma.

In some embodiments, the present disclosure provides a method of treating cancer in a subject, the method comprising administering to the subject a therapeutically effective amount of one or more compounds of the present disclosure (e.g., a compound of Formula I (e.g., Formula I-1, I-2, I-3, I-4, I-5, I-6, I-A, I-B, I-C, I-A-1, I-B-1, I-C-1, I-D, I-D-1, I-D-2, I-E, I-L-1, I-L-2, I-L-3, I-L-4, I-L-5, I-L-6, I-L-7, I-L-8, I-L-9, I-L-10, or I-L-11), II-1, I-2, II-3 (e.g., II-3-A, II-3-B, or II-3-C), II-4, II-5, II-6, II-7, III-1, III-2 (e.g., III-2-A, III-2-B, III-2-C, III-2-C-1, III-2-C-2, III-2-C-3, III-2-C-4, III-2-D, III-2-D-1, III-2-D-2, III-2-D-3, III-2-D-4, III-2-E-1, III-2-E-2, III-2-E-3, III-2-E-1a, III-2-E-2a, III-2-E-3a, III-2-E-1b, III-2-E-2b, or III-2-E-3b), III-3 (e.g., III-3-A), III-4 (e.g., III-4-A, III-4-B, III-4-C, III-4-D, III-4-C-1, III-4-C-2, III-4-C-3, III-4-C-4, III-4-D-1, or III-4-D-2), III-5, III-6, III-7, III-8 (e.g., III-8-A), III-9, IV, or V (e.g., V-1, V-2, or V-3), any of Compound Nos. 1-134, any compound selected from the compounds shown in Table A herein, or a pharmaceutically acceptable salt thereof) or a therapeutically effective amount of a pharmaceutical composition described herein. In some embodiments, the cancer is cancer of the colon, pancreatic cancer, breast cancer, prostate cancer, lung cancer, ovarian cancer, cervical cancer, renal cancer, cancer of the head and neck, lymphoma, leukemia, and/or melanoma. Types of cancers that may be treated with the compound of the present disclosure include, but are not limited to, brain cancers, skin cancers, bladder cancers, ovarian cancers, breast cancers, gastric cancers, pancreatic cancers, prostate cancers, colon cancers, blood cancers, lung cancers and bone cancers. Examples of such cancer types include neuroblastoma, intestine carcinoma such as rectum carcinoma, colon carcinoma, familiar adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, renal carcinoma, kidney parenchymal carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, diffuse large B-cell lymphoma (DLBCL), hepatocellular carcinoma, gall bladder carcinoma, bronchial carcinoma, small cell lung carcinoma, non-small cell lung carcinoma, multiple myeloma, basalioma, teratoma, retinoblastoma, choroid melanoma, seminoma, rhabdomyosarcoma, craniopharyngioma, osteosarcoma, chondrosarcoma, myosarcoma, liposarcoma, fibrosarcoma, Ewing sarcoma and plasmocytoma.

In some embodiments, the present disclosure provides a method of treating viral infection in a subject, the method comprising administering to the subject a therapeutically effective amount of one or more compounds of the present disclosure (e.g., a compound of Formula I (e.g., Formula I-1, I-2, I-3, I-4, I-5, I-6, I-A, I-B, I-C, I-A-1, I-B-1, I-C-1, I-D, I-D-1, I-D-2, I-E, I-L-1, I-L-2, I-L-3, I-L-4, I-L-5, I-L-6, I-L-7, I-L-8, I-L-9, I-L-10, or I-L-11), II-1, I-2, II-3 (e.g., II-3-A, II-3-B, or II-3-C), II-4, II-5, II-6, II-7, III-1, III-2 (e.g., III-2-A, III-2-B, III-2-C, III-2-C-1, III-2-C-2, III-2-C-3, III-2-C-4, III-2-D, III-2-D-1, III-2-D-2, III-2-D-3, III-2-D-4, III-2-E-1, III-2-E-2, III-2-E-3, III-2-E-1a, III-2-E-2a, III-2-E-3a, III-2-E-1b, III-2-E-2b, or III-2-E-3b), III-3 (e.g., III-3-A), III-4 (e.g., III-4-A, III-4-B, III-4-C, III-4-D, III-4-C-1, III-4-C-2, III-4-C-3, III-4-C-4, III-4-D-1, or III-4-D-2), III-5, III-6, III-7, III-8 (e.g., III-8-A), III-9, IV, or V (e.g., V-1, V-2, or V-3), any of Compound Nos. 1-134, any compound selected from the compounds shown in Table A herein, or a pharmaceutically acceptable salt thereof) or a therapeutically effective amount of a pharmaceutical composition described herein. Viral infections that may be treated include, but are not limited to, diseases caused by: hepatitis C virus (HCV), human papilloma virus (HPV), cytomegalovirus (CMV), herpes simplex virus (HSV), Epstein-Barr virus (EBV), varicella zoster virus, coxsackie virus, human immunodeficiency virus (HIV). In some embodiments, parasitic infections (e.g., malaria) may also be treated by the above methods.

In some embodiments, the present disclosure provides a method of treating a disease or disorder, e.g., a cancer associated with DGKa and/or DGKz in a subject in need thereof. In some embodiments, the method comprises administering to the subject a therapeutically effective amount of a compound of the present disclosure (e.g., a compound of Formula I (e.g., Formula I-1, I-2, I-3, I-4, I-5, I-6, I-A, I-B, I-C, I-A-1, I-B-1, I-C-1, I-D, I-D-1, I-D-2, I-E, I-L-1, I-L-2, I-L-3, I-L-4, I-L-5, I-L-6, I-L-7, I-L-8, I-L-9, I-L-10, or I-L-11), II-1, I-2, II-3 (e.g., II-3-A, II-3-B, or II-3-C), II-4, II-5, II-6, II-7, III-1, III-2 (e.g., III-2-A, III-2-B, III-2-C, III-2-C-1, III-2-C-2, III-2-C-3, III-2-C-4, III-2-D, III-2-D-1, III-2-D-2, III-2-D-3, III-2-D-4, III-2-E-1, III-2-E-2, III-2-E-3, III-2-E-1a, III-2-E-2a, III-2-E-3a, III-2-E-1b, III-2-E-2b, or III-2-E-3b), III-3 (e.g., III-3-A), III-4 (e.g., III-4-A, III-4-B, III-4-C, III-4-D, III-4-C-1, III-4-C-2, III-4-C-3, III-4-C-4, III-4-D-1, or III-4-D-2), III-5, III-6, III-7, III-8 (e.g., III-8-A), III-9, IV, or V (e.g., V-1, V-2, or V-3), any of Compound Nos. 1-134, any compound selected from the compounds shown in Table A herein, or a pharmaceutically acceptable salt thereof) or a therapeutically effective amount of a pharmaceutical composition described herein. In some embodiments, the cancer is cancer of the colon, pancreatic cancer, breast cancer, prostate cancer, lung cancer, ovarian cancer, cervical cancer, renal cancer, cancer of the head and neck, lymphoma, leukemia, and/or melanoma. Other types of cancer suitable to be treated by the method include those described herein.

Compounds of the present disclosure can be used as a monotherapy or in a combination therapy. In some embodiments, the combination therapy includes treating the subject with a targeted therapeutic agent, chemotherapeutic agent, therapeutic antibody, radiation, cell therapy, and/or immunotherapy. In some embodiments, compounds of the present disclosure can also be co-administered with an additional pharmaceutically active compound, either concurrently or sequentially in any order, to a subject in need thereof. In some embodiments, the combination therapy includes treating the subject with one or more additional therapies such as anti-viral agents, chemotherapeutics or other anti-cancer agents, immune enhancers, immunosuppressants, radiation, anti-tumor and anti-viral vaccines, cytokine therapy (e.g., IL2 and GM-CSF), and/or tyrosine kinase inhibitors.

In some embodiments, compounds of the present disclosure can be administered concurrently or sequentially in any order with an immuno-oncology agent. Immuno-oncology agents include, for example, a small molecule drug, antibody, or other biologic or small molecule. Examples of biologic immuno-oncology agents include, but are not limited to, cancer vaccines, antibodies, and cytokines. In some embodiments, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators). Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTbR, LIGHT, DcR3, HVEM, VEGI/TL1A, TRAMP/DR3, EDAR, EDA1, XEDAR, EDA2, TNFR1, Lymphotoxin a/TNFb, TNFR2, TNFa, LTbR, Lymphotoxin a 1b2, FAS, FASL, RELT, DR6, TROY, NGFR. T cell responses can be stimulated by a combination of a compound of the present disclosure and one or more of (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H. Other agents that can be combined with compounds of the present disclosure for the treatment of cancer include antagonists of inhibitory receptors on NK cells or agonists of activating receptors on NK cells. For example, a compound of the present disclosure can be combined with antagonists of KIR, such as lirilumab. Other agents for combination therapies include agents that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357). In some embodiments, a compound of the present disclosure can be used with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites. In some embodiments, the immuno-oncology agent is a CTLA-4 antagonist, such as an antagonistic CTLA-4 antibody. Suitable CTLA-4 antibodies include, for example, YERVOY (ipilimumab) or tremelimumab. In some embodiments, the immuno-oncology agent is a PD-1 antagonist, such as an antagonistic PD-1 antibody. Suitable PD-1 antibodies include, for example, OPDIVO (nivolumab), KEYTRUDA (pembrolizumab), or MEDI-0680 (AMP-514; WO2012/145493). The immuno-oncology agent may also include pidilizumab (CT-011), though its specificity for PD-1 binding has been questioned. Another approach to target the PD-1 receptor is the recombinant protein composed of the extracellular domain of PD-L2 (B7-DC) fused to the Fc portion of IgG1, called AMP-224. In some embodiments, the immuno-oncology agent is a PD-L1 antagonist, such as an antagonistic PD-L1 antibody. Suitable PD-L1 antibodies include, for example, MPDL3280A (RG7446; WO2010/077634), durvalumab (MEDI4736), BMS-936559 (WO2007/005874), and MSB0010718C (WO2013/79174). In some embodiments, the immuno-oncology agent is a LAG-3 antagonist, such as an antagonistic LAG-3 antibody. Suitable LAG3 antibodies include, for example, BMS-986016 (WO10/19570, WO14/08218), or IMP-731 or IMP-321 (WO08/132601, WO09/44273). In some embodiments, the immuno-oncology agent is a CD137 (4-1B1) agonist, such as an agonistic CD137 antibody. Suitable CD137 antibodies include, for example, urelumab and PF-05082566 (WO12/32433). In some embodiments, the immuno-oncology agent is a GITR agonist, such as an agonistic GITR antibody. Suitable GITR antibodies include, for example, BMS-986153, BMS-986156, TRX-518 (WO06/105021, WO09/009116) and MK-4166 (WO11/028683). In some embodiments, the immuno-oncology agent is an IDO antagonist. Suitable IDO antagonists include, for example, INCB-024360 (WO2006/122150, WO07/75598, WO08/36653, WO08/36642), indoximod, BMS-986205, or NLG-919 (WO09/73620, WO09/1156652, WO11/56652, WO12/142237). In some embodiments, the immuno-oncology agent is an OX40 agonist, such as an agonistic OX40 antibody. Suitable OX40 antibodies include, for example, MEDI-6383 or MEDI-6469. In another aspect, the immuno-oncology agent is an OX40L antagonist, such as an antagonistic OX40 antibody. Suitable OX40L antagonists include, for example, RG-7888 (WO06/029879). In some embodiments, the immuno-oncology agent is a CD40 agonist, such as an agonistic CD40 antibody. In yet another embodiment, the immuno-oncology agent is a CD40 antagonist, such as an antagonistic CD40 antibody. Suitable CD40 antibodies include, for example, lucatumumab or dacetuzumab. In some embodiments, the immuno-oncology agent is a CD27 agonist, such as an agonistic CD27 antibody. Suitable CD27 antibodies include, for example, varlilumab. In some embodiments, the immuno-oncology agent is MGA271 (to B7H3) (WO11/109400).

Combination therapy also can include the administration of the therapeutic agents as described above in further combination with other biologically active ingredients and/or non-drug therapies (e.g., surgery or radiation treatment.)

Suitable antiviral agents contemplated for use in combination with the compound of the present disclosure can comprise nucleoside and nucleotide reverse transcriptase inhibitors (NRTIs), non-nucleoside reverse transcriptase inhibitors (NNRTIs), protease inhibitors and other antiviral drugs. Examples of suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir; BCH-I0652; emitricitabine [(−)-FTC]; beta-L-FD4 (also called beta-L-D4C and named beta-L-2¢,3¢-dicleoxy-5-fluoro-cytidene); DAPD, ((−)-beta-D-2,6-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir; DMP-450; BMS-2322623; ABT-378; and AG-1549. Other antiviral agents include hydroxyurea, ribavirin, IL-2, IL-12, pentafuside and Yissum Project No. 11607.

Additional combination therapies such as additional immune-oncology agents also include any of those described as suitable for combination with DGK inhibitors in any of the following published patent applications: WO2019005883; WO2020006016; WO2020006018; WO2021041588; WO2021105115; WO2021105116; WO2021105117; WO2021127554; WO2021130638; WO2021132422; WO2021133748; WO2021133749; WO2021133750; WO2021133751; WO2021133752; WO2021214019; or WO2021214020.

The administering herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the administering is orally.

Dosing regimen including doses can vary and can be adjusted, which can depend on the recipient of the treatment, the disease or disorder being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

It is also meant to be understood that a specific embodiment of a variable moiety herein can be the same or different as another specific embodiment having the same identifier.

The present disclosure encompasses all combinations of the aspects and/or embodiments of the disclosure herein. It is understood that any and all embodiments of the present disclosure may be taken in conjunction with any other embodiment or embodiments to describe additional embodiments. It is also to be understood that each individual element of the embodiments is meant to be combined with any and all other elements from any embodiment to describe an additional embodiment.

Suitable atoms or groups for the variables herein are independently selected. The definitions of the variables can be combined. Using Formula I as an example, any of the definitions of one of R¹, R², R³, R⁴, U, X, Y, Z, L¹, L², and Ring A in Formula I can be combined with any of the definitions of the others of R¹, R², R³, R⁴, U, X, Y, Z, L¹, L², and Ring A in Formula I. Such combination is contemplated and within the scope of the present disclosure. Non-limiting useful groups for the variables in compounds of Formula I, II-1 to II-7, III-1 to III-9, IV or V, or a subformula thereof, as applicable, include any of the respective groups, individually or in any combination, as shown in the Examples or in the specific compounds described in Table A herein.

Although well understood in the field, to be clear, when a formula herein is said to be a sub-formula of a broader formula, it should be understood that for a variable(s) that is defined in connection with the broader formula, such variable(s) for the sub-formula can have the same definition as any of those defined for the broader formula, and the preferred definition of such variable(s) for the sub-formula can also include the same preferred definition as any of those described for the broader formula, unless obviously contrary from context. Similarly, for a variable(s) that is defined in connection with the sub-formula, such variable(s) for the broader formula or a different sub-formula of the broader formula can have the same definition as any of those defined for the sub-formula, unless obviously contrary from context.

The symbol, [symbol] when displayed perpendicular to (or otherwise crossing) a bond, indicates the point at which the displayed moiety is attached to the remainder of the molecule. It should be noted that for a divalent structure (or multivalent structure), the immediately connected group or groups or appropriate variable(s) shown in a formula maybe shown in the divalent structure (or multivalent structure) beyond the symbol, [symbol] , to indicate direction of attachment. When the immediately connected group(s) or variable is not shown for either of the two attaching points of a divalent structure, it should mean that either direction of attachment to the remainder of the molecule is allowed, unless otherwise specified or obviously contrary from context. Using a structure of “X-A-G-B” to illustrate, for example, if G is defined as

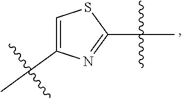 [SMILES: *c1csc(*)n1]

i.e., the immediately connected group(s) or variable(s) is not shown, then the structure of “X-A-G-B” can be either

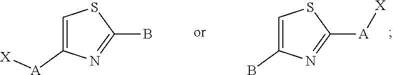

on the other hand, if G is defined as

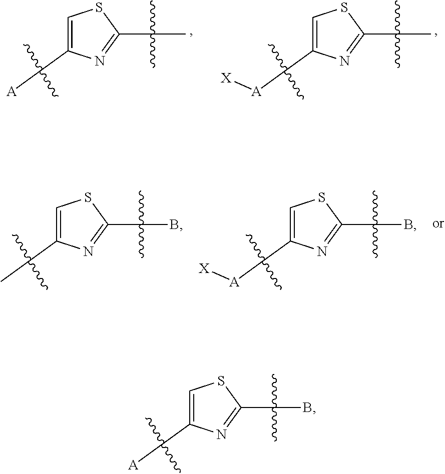

then the structure of “X-A-G-B” should be understood as

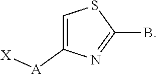

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds of the present disclosure can comprise one or more asymmetric centers and/or axial chirality, and thus can exist in various isomeric forms, e.g., enantiomers and/or diastereomers. For example, the compounds described herein can be in the form of an individual enantiomer, diastereomer, atropisomer, or geometric isomer, or can be in the form of a mixture of stereoisomers, including racemic mixtures and mixtures enriched in one or more stereoisomer. Isomers can be isolated from mixtures by methods known to those having ordinary skill in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures. In embodiments herein, unless otherwise obviously contrary from context, when a stereochemistry is specifically drawn, it should be understood that with respect to that particular chiral center or axial chirality, the compound can exist predominantly as the as-drawn stereoisomer, such as with less than 20%, less than 10%, less than 5%, less than 1%, by weight, by HPLC or SFC area, or both, or with a non-detectable amount of the other stereoisomer(s), for example, the compound can be characterized as having an enantiomeric excess (“ee”) of greater than 60%, such as greater than 80% ee, greater than 90% ee, greater than 95% ee, greater than 98% ee, or greater than 99% ee. The presence and/or amounts of stereoisomers can be determined by those having ordinary skill in the art in view of the present disclosure, including through the use of chiral HPLC or SFC. When the stereochemistry of a chiral center is not specifically drawn, it should be understood that the structure or a fragment thereof is intended to encompass all possible stereoisomers with respect to that particular chiral center.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the term “compound(s) of the present disclosure” or “compound(s) of the present invention” refers to any of the compounds described herein according to Formula I (e.g., Formula I-1, I-2, I-3, I-4, I-5, I-6, I-A, I-B, I-C, I-A-1, I-B-1, I-C-1, I-D, I-D-1, I-D-2, I-E, I-L-1, I-L-2, I-L-3, I-L-4, I-L-5, I-L-6, I-L-7, I-L-8, I-L-9, I-L-10, or I-L-11), II-1, I-2, II-3 (e.g., II-3-A, II-3-B, or II-3-C), II-4, II-5, II-6, II-7, III-1, III-2 (e.g., III-2-A, III-2-B, III-2-C, III-2-C-1, III-2-C-2, III-2-C-3, III-2-C-4, III-2-D, III-2-D-1, III-2-D-2, III-2-D-3, III-2-D-4, III-2-E-1, III-2-E-2, III-2-E-3, III-2-E-1a, III-2-E-2a, III-2-E-3a, III-2-E-1b, III-2-E-2b, or III-2-E-3b), III-3 (e.g., III-3-A), III-4 (e.g., III-4-A, III-4-B, III-4-C, III-4-D, III-4-C-1, III-4-C-2, III-4-C-3, III-4-C-4, III-4-D-1, or III-4-D-2), III-5, III-6, III-7, III-8 (e.g., III-8-A), III-9, IV, or V (e.g., V-1, V-2, or V-3), any of Compound Nos. 1-134, any compound selected from the compounds shown in Table A herein, isotopically labeled compound(s) thereof (such as a deuterated analog wherein one or more of the hydrogen atoms is substituted with a deuterium atom with an abundance above its natural abundance), possible stereoisomers thereof (including diastereoisomers, enantiomers, and racemic mixtures), geometric isomers thereof, atropisomers thereof, tautomers thereof, conformational isomers thereof, and/or pharmaceutically acceptable salts thereof (e.g., acid addition salt such as HCl salt or base addition salt such as Na salt). For the avoidance of doubt, Compound Nos. 1-134 or Compounds 1-134 refers to the compounds described herein labeled as integers 1, 2, 3, . . . , 134, see for example the title compounds of Examples 1-37 and Table 1. For ease of description, synthetic starting materials or intermediates may be labeled with an integer (compound number) followed by a “-” and additional numeric values, such as 2-1, 2-2, etc., see examples for details. The labeling of such synthetic starting materials or intermediates should not be confused with the compounds labeled with an integer only without the “-” and additional numeric value. Hydrates and solvates of the compounds of the present disclosure are considered compositions of the present disclosure, wherein the compound(s) is in association with water or solvent, respectively. In some embodiments, the compound of the present disclosure can be any of those defined in claims 1-191 herein. In some embodiments, the compound of the present disclosure can be any of those defined in any of the enumerated embodiments A1-A26 and B1-B21.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

As used herein, the term “alkyl” as used by itself or as part of another group refers to a straight- or branched-chain aliphatic saturated hydrocarbon. In some embodiments, the alkyl which can include one to twelve carbon atoms (i.e., C₁₋₁₂ alkyl) or the number of carbon atoms designated (i.e., a C₁ alkyl such as methyl, a C₂ alkyl such as ethyl, a C₃ alkyl such as propyl or isopropyl, etc.). In one embodiment, the alkyl group is a straight chain C₁₋₁₀ alkyl group. In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. In one embodiment, the alkyl group is a C₁₋₄ alkyl group selected from methyl, ethyl, propyl (n-propyl), isopropyl, butyl (n-butyl), sec-butyl, tert-butyl, and iso-butyl. As used herein, the term “alkylene” as used by itself or as part of another group refers to a divalent radical derived from an alkyl group. For example, non-limiting straight chain alkylene groups include —CH₂—CH₂—CH₂—CH₂—, —CH₂—CH₂—CH₂—, —CH₂—CH₂—, and the like.

As used herein, the term “alkenyl” as used by itself or as part of another group refers to a straight- or branched-chain aliphatic hydrocarbon containing one or more, such as one, two or three carbon-to-carbon double bonds. In one embodiment, the alkenyl group is a C₂₋₆ alkenyl group. In another embodiment, the alkenyl group is a C₂4 alkenyl group. Non-limiting exemplary alkenyl groups include ethenyl, propenyl, isopropenyl, butenyl, sec-butenyl, pentenyl, and hexenyl.

As used herein, the term “alkynyl” as used by itself or as part of another group refers to a straight- or branched-chain aliphatic hydrocarbon containing one or more, such as one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂4 alkynyl group. Non-limiting exemplary alkynyl groups include ethynyl, propynyl, butynyl, 2-butynyl, pentynyl, and hexynyl groups.

As used herein, the term “alkoxy” as used by itself or as part of another group refers to a radical of the formula OR^{a1}, wherein R^{a1} is an alkyl. As used herein, the term “cycloalkoxy” as used by itself or as part of another group refers to a radical of the formula OR^{a1}, wherein R^{a1} is a cycloalkyl.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one or more fluorine atoms, alternatively referred to herein as fluorine-substituted alkyl, such as with one, two, or three fluorine atoms. In one embodiment, the haloalkyl group is a C₁₋₄ haloalkyl group. In one embodiment, the haloalkyl group is a fluorine-substituted C₁₋₄ alkyl group.

As used herein, the term “heteroalkyl,” by itself or in combination with another term, means, unless otherwise stated, a stable straight or branched-chain alkyl group, e.g., having from 2 to 14 carbons, such as 2 to 10 carbons in the chain, one or more of the carbons has been replaced by a heteroatom selected from S, O, P and N, and wherein the nitrogen, phosphine, and sulfur atoms can optionally be oxidized and the nitrogen heteroatom can optionally be quaternized. The heteroatom(s) S, O, P and N may be placed at any interior position of the heteroalkyl group or at the position at which the alkyl group is attached to the remainder of the molecule. When the heteroalkyl is said to be substituted, the substituent(s) can replace one or more hydrogen atoms attached to the carbon atom(s) and/or the heteroatom(s) of the heteroalkyl. In some embodiments, the heteroalkyl is a C₁₋₄ heteroalkyl, which refers to the heteroalkyl defined herein having 1-4 carbon atoms. Examples of C₁4 heteroalkyl include, but are not limited to, C₄ heteroalkyl such as —CH₂—CH₂—N(CH₃)—CH₃, C₃ heteroalkyl such as —CH₂—CH₂—O—CH₃, —CH₂—CH₂—NH—CH₃, —CH₂—S—CH₂—CH₃, —CH₂—CH₂—S(O)—CH₃, —CH₂—CH₂—S(O)₂—CH₃, C₂ heteroalkyl such as —CH₂—CH₂—OH, —CH₂—CH₂—NH₂, —CH₂—NH(CH₃), —O—CH₂—CH₃ and C₁ heteroalkyl such as, —CH₂—OH, —CH₂—NH₂, —O—CH₃. Similarly, the term “heteroalkylene” by itself or as part of another substituent means a divalent radical derived from heteroalkyl, as exemplified, but not limited by, —CH₂—CH₂—O—CH₂—CH₂— and —O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. Where “heteroalkyl” is recited, followed by recitations of specific heteroalkyl groups, such as —NR⁸R¹¹ or the like, it will be understood that the terms heteroalkyl and —NR⁸R¹¹ are not redundant or mutually exclusive. Rather, the specific heteroalkyl groups are recited to add clarity. Thus, the term “heteroalkyl” should not be interpreted herein as excluding specific heteroalkyl groups, such as —NR⁸R¹¹ or the like.

“Carbocyclyl” or “carbocyclic” as used by itself or as part of another group refers to a radical of a non-aromatic cyclic hydrocarbon group having from 3 to 10 ring carbon atoms (“C₃₋₁₀ carbocyclyl”) and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Non-limiting exemplary carbocyclyl groups include cyclopropyl, cyclobutyl, cyclopentyl, cyclohexyl, cycloheptyl, cyclooctyl, norbornyl, decalin, adamantyl, cyclopentenyl, and cyclohexenyl.

In some embodiments, “carbocyclyl” is fully saturated, which is also referred to as cycloalkyl. In some embodiments, the cycloalkyl can have from 3 to 10 ring carbon atoms (“C₃₋₁₀ cycloalkyl”). In preferred embodiments, the cycloalkyl is a monocyclic ring.

“Heterocyclyl” or “heterocyclic” as used by itself or as part of another group refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). Heterocyclyl or heterocyclic ring that has a ring size different from the 3-10 membered heterocyclyl is specified with a different ring size designation when applicable. Those having ordinary skill in the art would understand that such different ring-sized heterocyclyl is also a non-aromatic ring system having ring carbon atoms and 1 to 4 ring heteroatoms, wherein each heteroatom is independently selected from nitrogen, oxygen, sulfur, boron, phosphorus, and silicon. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). Heteroaryl that has a ring size different from the 5-10 membered heteroaryl is specified with a different ring size designation when applicable. Those having ordinary skill in the art would understand that such different ring-sized heteroaryl is also a 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur. In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, and the like) the point of attachment can be on either ring, i.e., either the ring bearing a heteroatom (e.g., 2-indolyl) or the ring that does not contain a heteroatom (e.g., 5-indolyl).

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzothiazolyl, benzisothiazolyl, benzothiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

As commonly understood in the art, alkylene, alkenylene, alkynylene, heteroalkylene, carbocyclylene, heterocyclylene, arylene, and heteroarylene refer to the corresponding divalent radicals of alkyl, alkenyl, alkynyl, heteroalkyl, carbocyclyl, heterocyclyl, aryl, and heteroaryl groups, respectively.

An “optionally substituted” group, such as an optionally substituted alkyl, optionally substituted alkenyl, optionally substituted alkynyl, optionally substituted heteroalkyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkenyl, alkynyl, heteroalkyl, carbocyclic, cycloalkyl, alkoxy, cycloalkoxy, or heterocyclic group herein can be unsubstituted or substituted with 1, 2, 3, or 4 substituents independently selected from F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, the “optionally substituted” aryl or heteroaryl group herein can be unsubstituted or substituted with 1, 2, 3, or 4 substituents independently selected from F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C₁₋₄ alkyl), C₁₋₄ alkyl, C₂₋₄ alkenyl, C₂₋₄ alkynyl, C₁4 alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR⁸, —OSO₂R⁸, —S(═O)R^{aa}, —OS(═O)R^{aa}, —Si(R^{aa})₃, —OSi(R^{aa})₃—C(═S)N(R^{bb})₂, —C(═O)SR—, —C(═S)SR^{aa}, —SC(═S)SR—, —SC(═O)SR^{aa}, —OC(═O)SR^{aa}, —SC(═O)OR^{aa}, —SC(═O)R^{aa}, —P(═O)(R^{a})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{b})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR⁸(OR^{cc}), C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; wherein X is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; each instance of R^{a} is, independently, selected from C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R⁸, —SO₂R¹¹, —C(═NR^{cc})OR^{aa}, —C(═NR^{cc})N(R^{cc})₂, —SO₂N(R^{C})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{a}a)₂, —P(═O)(OR^{cc})₂, —P(═O)(N(R^{cc})₂)₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; wherein X is a counterion;
each instance of R^{cc} is, independently, selected from hydrogen, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{cc}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{cc}, —OC(═O)R^{ee}, —OCO₂R^{ee}, —C(═O)N(R^{E})₂, —OC(═O)N(R^{ff})₂, —NR^{ff}C(═O)R^{ee}, —NR^{ff}CO₂R^{ee}, —NRC(═O)N(R^{E})₂, —C(═NR^{ff})OR^{ee}, —OC(═NR^{ff})R^{ee}, —OC(═NR^{ff})OR^{ee}, —C(═NR^{ff})N(R^{ee})₂, —OC(═NR^{ff})N(R^{ff})₂, —NR^{ff}(═NR^{ff})N(R^{ff})₂, —NR⁸SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{K})₂, —C(═O)SR^{ee}, —C(═S)SR^{ee}, —SC(═S)SR^{ee}, —P(═O)(OR^{ee})₂, —P(═O)(R^{ee})₂—OP(═O)(R^{ee})₂, —OP(═O)(OR^{ee})₂, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆-10 aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X is a counterion;
each instance of R^{ee} is, independently, selected from C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R⁸ groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C-6 alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻, NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, BPh₄⁻, Al(OC(CF₃)₃)₄⁻, and a carborane anion (e.g., CB₁₁H₁₂⁻ or (HCB₁₁Me₅Br₆)⁻). Exemplary counterions which may be multivalent include CO₃²⁻, HPO₄²⁻, P₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, Sali cylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

“Acyl” refers to a moiety selected from the group consisting of —C(═O)R^{aa}, —CHO, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —C(═O)NR^{bb}SO₂R^{aa}, —C(═S)N(R^{bb})₂, —C(═O)SR^{aa}, or —C(═S)SR^{aa}, wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{aa})N(R^{bb})₂, —SO₂N(R^{aa})₂, —SO₂R^{cc}, —SO₂OR^{cc}, —SOR^{a}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined above.

In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, —R^{aa}, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{a}, —SO₂R⁸, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, alkyl ethers or substituted alkyl ethers such as methyl, allyl, benzyl, substituted benzyls such as 4-methoxybenzyl, methoxymethyl (MOM), benzyloxymethyl (BOM), 2-methoxyethoxymethyl (MEM), etc., silyl ethers such as trymethylsilyl (TMS), triethylsilyl (TES), triisopropylsilyl (TIPS), t-butyldimethylsilyl (TBDMS), etc., acetals or ketals, such as tetrahydropyranyl (THP), esters such as formate, acetate, chloroacetate, dichloroacetate, trichloroacetate, trifluoroacetate, methoxyacetate, etc., carbonates, sulfonates such as methanesulfonate (mesylate), benzylsulfonate, and tosylate (Ts), etc.

The term “leaving group” is given its ordinary meaning in the art of synthetic organic chemistry, for example, it can refer to an atom or a group capable of being displaced by a nucleophile. See, for example, Smith, March Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/of” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### Examples

The various starting materials, intermediates, and compounds of the preferred embodiments can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. The examples are illustrative only and do not limit the claimed invention in any way.

Exemplary embodiments of steps for performing the synthesis of products described herein are described in greater detail infra. Some of the Examples discussed herein can be prepared by separating the corresponding racemic mixtures. As would be understood by a person of ordinary skill in the art, the compounds described in the Examples section immediately prior to the chiral separation step, e.g., by supercritical fluid chromatography (SFC), exist in racemic and/or stereoisomeric mixture forms. It should be understood that the enantiomeric excesses (“ee”) and/or diastereomeric excesses (“de”) reported for these examples are only representative from the exemplified procedures herein and not limiting; those of ordinary skill in the art would understand that such enantiomers and/or diastereomers with a different ee and/or de, such as a higher ee and/or de, can be obtained in view of the present disclosure. Typically, a “de” value is reported herein when a pair of diastereomers, having only one of the chiral centers being different, are separated from a corresponding diastereomeric mixture. In such cases, the “de” value indicates the degree of enrichment of one of the diastereomers.

The abbreviations used in the Examples section should be understood as having their ordinary meanings in the art unless specifically indicated otherwise or obviously contrary from context. The following shows certain abbreviations used in the Examples section herein.

| Abbreviations | Chemical Name |
|---|---|
| (Boc)₂O | di-tert-butyl dicarbonate |
| AcOH/HOAc | Acetic acid |
| AcONa/NaOAc | Sodium acetate |
| AIBN | 2,2′-Azobis(2-methylpropionitrile) |
| BINAP | racemic-2,2′-Bis(diphenylphosphino)-1,1′-binaphthyl |
| CDI | Di(1H-imidazol-1-yl)methanone |
| DAST | Diethylaminosulphur trifluoride |
| DBU | 1,8-Diazabicyclo[5.4.0]undec-7-ene |
| DCM | dichloromethane |
| DEA | Diethylamine |
| DEAD | DiethylAzodicarboxylate |
| DIAD | Diisopropyl azodicarboxylate |
| DIPEA | N,N-Diisopropylethylamine |
| DMAc | N,N-dimethylacetamide |
| DMAP | 4-Dimethylaminopyridine |
| DMF | N,N-dimethylformamide |
| DMSO | Dimethyl sulfoxide |
| dppf | 1,1-Bis(diphenylphosphino)ferrocene |
| EDCI | N-Ethyl-N′-(3-dimethylaminopropyl)carbodiimide hydrochloride |
| EtOH/ETOH | ethanol |
| FA | Formic acid |
| Grubbs 2nd | [1,3-bis(2,4,6-trimethylphenyl)imidazolidin-2-ylidene](dichloro){[2-(1- |
| catalyst | methylethoxy)phenyl]methylidene}ruthenium |
| HATU | O-(7-Azabenzotriazol-1-yl)-N,N,N′,N′-tetramethyluronium |
| | hexafluorophosphate |
| IPA | propan-2-ol |
| KHMDS | Potassium bis(trimethylsilyl)amide |
| KOAc | Potassium acetate |
| LDA | Lithium diisopropylamide |
| mCPBA | 3-Chloroperbenzoic acid |
| Me4t-BuXPhos | 2-Di-t-butylphosphino-3,4,5,6-tetramethyl-2′,4′,6′-tri-i-propyl)-1,1′-biphenyl |
| MeCN | acetonitrile |
| MeOH/MEOH | methanol |
| NaBH(OAc)₃ | Sodium triacetoxyborohydride |
| NBS | N-Bromosuccinimide |
| n-BuLi | n-butyllithium |
| NCS | N-Chlorosuccinimide |
| NMP | N-methylpyrrolidin-2-one |
| Pd(dppf)Cl₂ | [1,1′-Bis(diphenylphosphino)ferrocene]dichloropalladium(II) |
| Pd(OAc)₂ | Palladium (II) acetate |
| Pd(PPh₃)₄ | Tetrakis(triphenylphosphine)palladium |
| Pd2(dba)3 | Tris(dibenzylideneacetone)dipalladium(0) |
| PPA | polyphosphoric acid |
| PPh3 | triphenylphosphine |
| Rh(PPh₃)₃Cl | Tris(triphenylphosphine)chlororhodium |
| Ruphos Pd G3 | (2-Dicyclohexylphosphino-2′,6′-diisopropoxy-1,1′-biphenyl)[2-(2′-amino- |
| | 1,1′-biphenyl)]palladium(II) methanesulfonate |
| SEM | 2-(Trimethylsilyl)ethoxymethyl |
| SEMCl | 2-(Trimethylsilyl)ethoxymethyl Chloride |
| SFC | Supercritical Fluid Chromatography |
| TBAF | Tetra-n-butylammonium fluoride |
| TBAI | Tetrabutylammonium iodide |
| TBDPSCl | tert-Butylchlorodiphenylsilane |
| TBSOTf | tert-Butyldimethylsilyl trifluoromethanesulfonate |
| t-BuBrettPhos | 2-(Di-t-butylphosphino)-3,6-dimethoxy-2′,4′,6′-tri-i-propyl-1,1′-biphenyl |
| t-BuONO | tert-Butyl nitrite |
| TEA | Triethylamine |
| Tf2O | Trifluoromethanesulfonic anhydride |
| TFA | Trifluoroacetic acid |
| TFAA | Trifluoroacetic anhydride |
| THF | Tetrahydrofuran |
| TIPS | Triisopropylsilyl |
| TIPSCl | Triisopropylsilyl Chloride |
| TMSCN | Trimethylsilyl cyanide |
| TMSI | Trimethyliodosilane |
| Xantphos | 9,9-Dimethyl-4,5-bis(diphenylphosphino)xanthene |

### Example 1 Synthesis of Compound 1

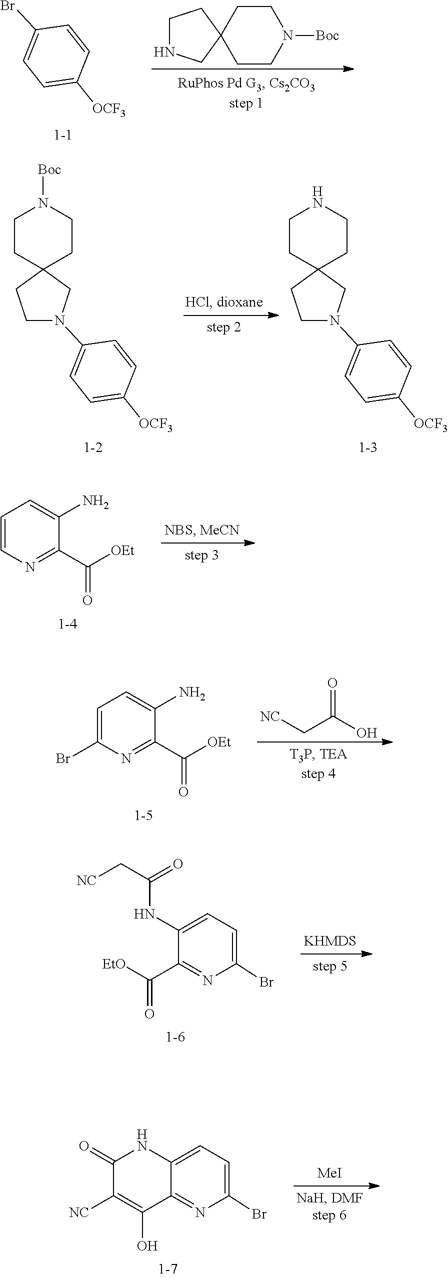

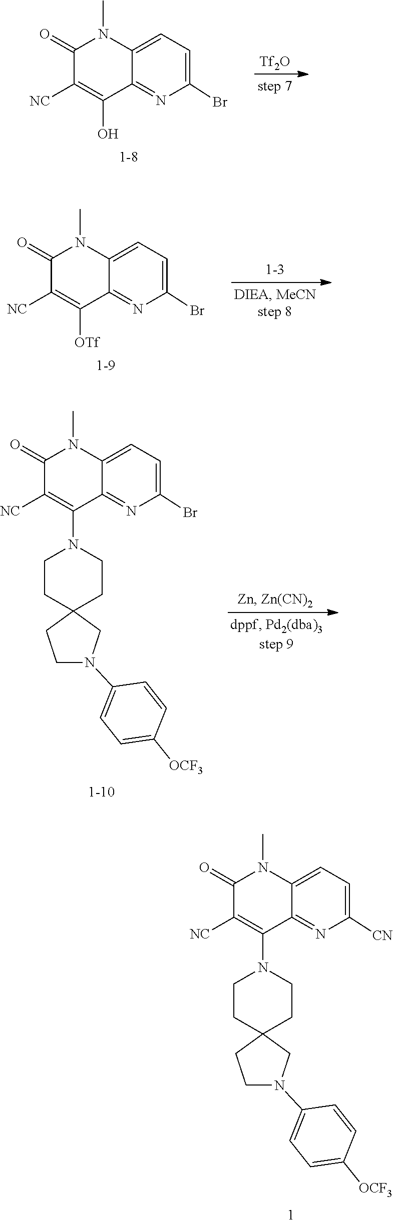 [SMILES: "Tf2O" ; Cn1c(=O)c(C#N)c(O)c2nc(Br)ccc21 ; Cn1c(=O)c(C#N)c(OS(=O)(=O)C(F)(F)F)c2nc(Br)ccc21 ; "1-3" ; Cn1c(=O)c(C#N)c(N2CCC3(CCN(c4ccc(OC(F)(F)F)cc4)C3)CC2)c2nc(Br)ccc21 ; Cn1c(=O)c(C#N)c(N2CCC3(CCN(c4ccc(OC(F)(F)F)cc4)C3)CC2)c2nc(C#N)ccc21]

Step 1: To a solution of 1-1 (2.5 g, 10.4 mmol) in dioxane (20 mL) was added tert-butyl 2,8-diazaspiro[4.5]decane-8-carboxylate (3.0 g, 12.45 mmol), Cs₂CO₃ (6.76 g, 20.75 mmol) and RuPhos Pd G3 (867.6 mg, 1.4 mmol). The resulting mixture was degassed with N₂ for 10 minutes, then stirred at 120° C. for 4 h. After cooled down to room temperature, the mixture was filtered and the filtrate was concentrated. The residue was purified by column chromatography on silica gel (petroleum ether/ethyl acetate=1/0 to 3/1) to afford 1-2.

Step 2: To a solution of 1-2 (2.8 g, 7 mmol) in dioxane (20 mL) was added HCl (4.3 mL, 43 mmol). The reaction mixture was stirred at room temperature for 5 h. The pH was adjusted to 6 by addition of saturated NaHCO₃. The mixture was extracted with ethyl acetate. The organic layer was dried over Na₂SO₄, filtered and the filtrate was concentrated. The residue was purified by column chromatography on silica gel (petroleum ether/ethyl acetate=1/0 to 0/1) to afford 1-3.

Step 3: To a solution of 1-4 (24 g, 144.4 mmol) in acetonitrile (250 mL) was added NBS (28.3 g, 158.9 mmol) at room temperature. The mixture was stirred at room temperature for 1 h. Water was added, and the mixture was extracted with ethyl acetate. The combined organic layers were dried over Na₂SO₄, filtered and the filtrated was concentrated. The residue was purified by column chromatography on silica gel (petroleum ether/ethyl acetate=1/0 to 0/1) to afford 1-5.

Step 4: To a solution of 2-cyanoacetic acid (16.6 g, 195.2 mmol) in DMF (400 mL) were added HATU (1.39 g, 3.7 mmol) and triethylamine (45.2 mL, 325.4 mmol) at room temperature. The mixture was stirred at room temperature for 15 minutes. Then 1-5 (31.9 g, 130.2 mmol) was added. The mixture was stirred at room temperature for 12 h. Water was added to the reaction mixture. The solid was collected by filtration to afford 1-6.

Step 5: To a solution of 1-6 (21.5 g, 68.9 mmol) in THF (300 mL) was added KHMDS (1M in THF, 89.6 mL, 89.6 mmol) at room temperature. The mixture was stirred at room temperature for 12 h. HCl (1 N) was added until the pH was 4. The solid was collected by filtration to afford 1-7.

Step 6: To a solution of 1-7 (15.8 g, 59.4 mmol) in DMF (200 mL) was added NaH (9.5 g, 60%, 237.6 mmol) in portions at 0° C. The mixture was stirred at 0° C. for 30 minutes. CH₃I (14.8 mL, 237.6 mmol) was added. The reaction was stirred at room temperature for 12 h, then quenched with H₂O. The pH of the reaction mixture was adjusted to 3 with HCl (1 N). The solid was collected by filtration to afford 1-8.

Step 7: To a solution of 1-8 (17.3 g, 61.8 mmol), triethylamine (25.8 mL, 185.3 mmol) and DMAP (0.8 g, 6.2 mmol) in dichloromethane (200 mL) was added Tf₂O (34.9 g, 123.5 mmol) at 0° C. The mixture was stirred at 0° C. for 0.5 h, then quenched with H₂O and extracted with dichloromethane. The combined organic layers were dried over Na₂SO₄, filtered and the filtrate was concentrated. The residue was purified by column chromatography on silica gel (petroleum ether/ethyl acetate=1/0 to 2/1) to afford 1-9.

Step 8: To a mixture of 1-9 (300 mg, 0.73 mmol) and 1-3 (327.9 mg, 1.1 mmol) in acetonitrile (10 mL) was added DIPEA (0.36 mL, 2.18 mmol). The mixture was stirred at 85° C. for 2 h. The reaction was quenched by H₂O. The aqueous layer was extracted with ethyl acetate. The organic layers were combined and washed with brine, dried over anhydrous Na₂SO₄, filtered and the filtrate was concentrated. The residue was purified by column chromatography on silica gel (petroleum ether/ethyl acetate=1/1) to afford 1-10.

Step 9: To a solution of 1-10 (210 mg, 0.37 mmol) in NMP (8 mL) were added Zn(CN)₂ (87.7 mg, 0.75 mmol), Zn (4.9 mg, 0.075 mmol), Pd₂(dba)₃ (34.2 mg, 0.037 mmol) and dppf (12.6 mg, 0.022 mmol) under N₂. The reaction mixture was stirred at 80° C. for 1 h. The reaction was quenched by H₂O. The aqueous layer was extracted with ethyl acetate. The combined organic layers were washed with brine, dried over anhydrous Na₂SO₄, filtered and the filtrate was concentrated. The residue was purified by reverse phase HPLC (Reverse phase HPLC (acetonitrile/water: 5-95%) to afford 1. LCMS (ESI, m/z): [M+H]⁺=509.4; ¹H NMR (400 MHz, DMSO-d₆, ppm): δ 8.25 (d, J=8.8 Hz, 1H), 8.14 (d, J=8.8 Hz, 1H), 7.12 (d, J=8.4 Hz, 2H), 6.58 (d, J=9.2 Hz, 2H), 3.90-3.85 (m, 4H), 3.52 (s, 3H), 3.38-3.34 (m, 2H), 3.25 (s, 2H), 1.99 (t, J=6.8 Hz, 2H), 1.86-1.80 (m, 4H).

### Example 2 Synthesis of Compound 2

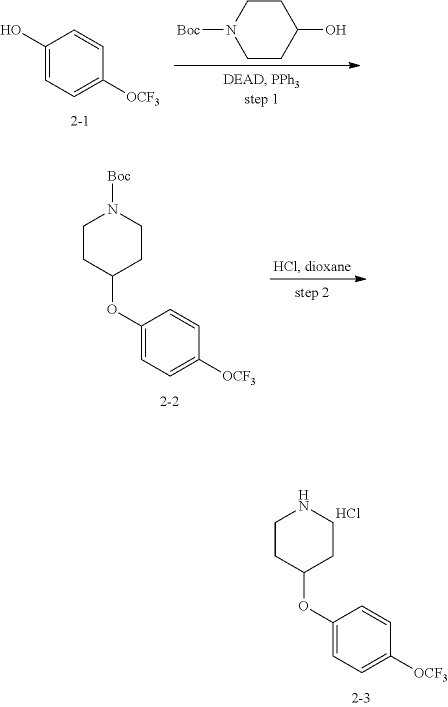 [SMILES: Oc1ccc(OC(F)(F)F)cc1 ; CC(C)(C)OC(=O)N1CCC(O)CC1 ; CC(C)(C)OC(=O)N1CCC(Oc2ccc(OC(F)(F)F)cc2)CC1 ; FC(F)(F)Oc1ccc(OC2CCNCC2)cc1 ; Cl]

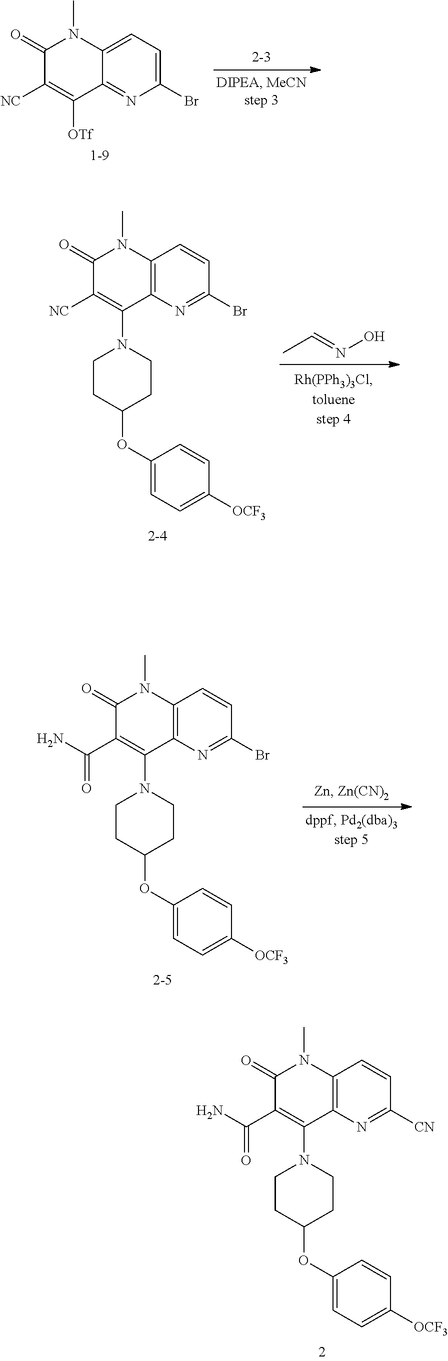

Step 1: To a solution of 2-1 (19 g, 106.7 mmol), tert-butyl 4-hydroxypiperidine-1-carboxylate (21.5 g, 106.7 mmol) and PPh₃ (30.8 g, 117.3 mmol) in THF (85 mL) was added DEAD (18.6 mL, 117.3 mmol) at 0° C. under N₂. The mixture was stirred at 20° C. under N₂ for 16 h. The mixture was concentrated. The residue was purified by column chromatography on silica gel (petroleum ether/ethyl acetate=1/0 to 4/1) to afford 2-2.

Step 2: To a solution of 2-2 (22.1 g, 61.2 mmol) in dichloromethane (20 mL) was added a solution of HCl in dioxane (4M, 61.2 mL, 244.8 mmol) at 20° C. The mixture was stirred at 20° C. for 16 h. The mixture was concentrated under reduced pressure to afford 2-3.

Step 3: To a mixture of 1-9 (600 mg, 1.46 mmol) and 2-3 (456.4 mg, 1.75 mmol) in acetonitrile (10 mL) was added DIPEA (564.4 mg, 4.37 mmol) at room temperature. The mixture was stirred at 85° C. for 1 h. This mixture was concentrated and the residue was purified by column chromatography on silica gel (petroleum ether/ethyl acetate=1/0 to 10/1) to afford 2-4.

Step 4: To a solution of 2-4 (300 mg, 0.57 mmol) and acetaldoxime (609.5 mg, 10.3 mmol) in toluene (15 mL) was added Rh(PPh₃)₃Cl (53.0 mg, 0.057 mmol) at room temperature. Then the mixture was heated at 110° C. for 12 h. The mixture was concentrated and the residue was purified by column chromatography on silica gel (petroleum ether/ethyl acetate=1/0 to 10/1) to afford 2-5.

Step 5: To a mixture of 2-5 (250 mg, 0.46 mmol), Zn (6.0 mg, 0.09 mmol) and Zn(CN)₂ (108.5 mg, 0.92 mmol) in NMP (4 mL) were added Pd₂(dba)₃ (42.3 mg, 0.046 mmol) and dppf (12.8 mg, 0.023 mmol) at room temperature. Then the mixture was stirred at 90° C. for 1 h. The reaction mixture was quenched with H₂O and extracted with ethyl acetate. The combined organic layers were dried over Na₂SO₄, filtered and the filtrate was concentrated. The residue was purified by reverse phase HPLC (acetonitrile/0.05% TFA in water: 5%-80%) to afford 2 as 0.2 eq of TFA salt. LCMS (ESI, m/z): [M+H]⁺=488.4; ¹H NMR (400 MHz, DMSO-d₆, ppm): δ 8.17 (d, J=8.8 Hz, 1H), 8.08 (d, J=8.9 Hz, 1H), 7.65 (s, 1H), 7.50 (s, 1H), 7.27 (d, J=8.6 Hz, 2H), 7.13-7.08 (m, 2H), 4.73-4.65 (m, 1H), 3.63 (s, 2H), 3.55 (s, 3H), 3.34-3.29 (m, 2H), 2.13-2.10 (m, 2H), 1.86-1.83 (m, 2H). ¹⁹F NMR (376 MHz, DMSO-d₆, ppm): δ −57.25 (3F), −74.58 (0.6F, TFA).

### Example 3 Synthesis of Compound 3

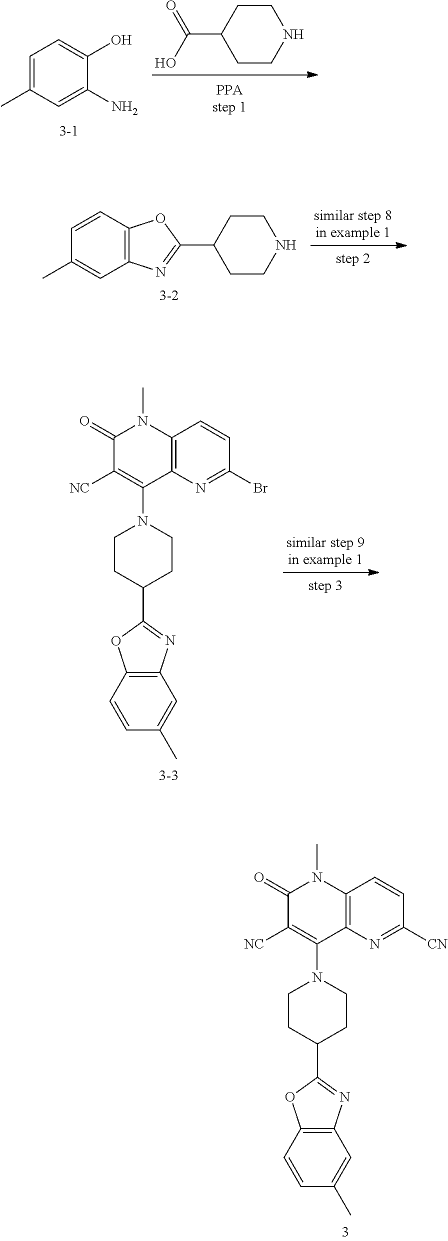

Step 1: 3-1 (10 g, 81.2 mmol) and piperidine-4-carboxylic acid (10.5 g, 81.2 mmol) were added to PPA (53.4 g, 81.2 mmol) and the mixture was stirred at 180° C. for 2 h. The reaction mixture was cooled to 90° C., then H₂O was added to quench the reaction. The pH of the mixture was adjusted to 12 with 50% KOH. The mixture was extracted with CH₂Cl₂. The organic layer was dried over Na₂SO₄, filtered and concentrated to afford 3-2.

Compound 3-3 was prepared from compound 3-2 following the procedure for the synthesis of compound 1-10 in example 1.

Compound 3 was prepared from compound 3-3 following the procedure for the synthesis of compound 1 in example 1. LCMS (ESI, m/z): [M+H]⁺=425.2; ¹H NMR (400 MHz, DMSO-d₆, ppm) δ 8.27-8.24 (m, 1H), 8.16-8.13 (m, 1H), 7.54-7.56 (m, 1H), 7.51 (s, 1H), 7.17-7.15 (m, 1H), 4.28-4.24 (m, 2H), 3.67 (t, J=11.2 Hz, 2H), 3.53 (s, 3H), 3.50-3.46 (m, 1H), 2.40 (s, 3H), 2.30-2.28 (m, 2H), 2.16-2.08 (m, 2H).

### Example 4 Synthesis of Compound 4

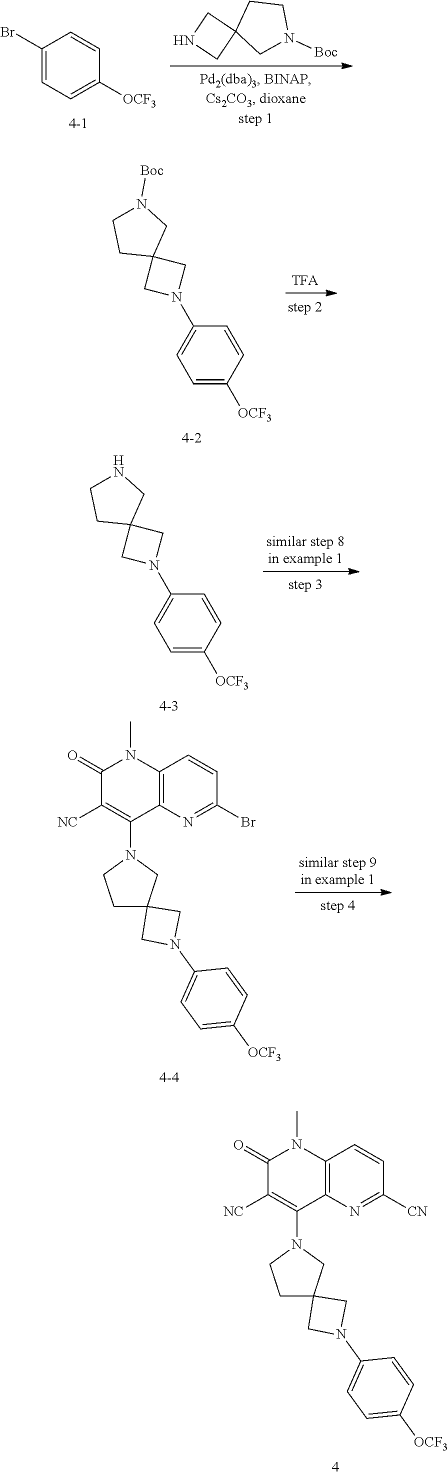 [SMILES: FC(F)(F)Oc1ccc(Br)cc1 ; CC(C)(C)OC(=O)N1CCC2(CNC2)C1 ; CC(C)(C)OC(=O)N1CCC2(C1)CN(c1ccc(OC(F)(F)F)cc1)C2 ; FC(F)(F)Oc1ccc(N2CC3(CCNC3)C2)cc1 ; Cn1c(=O)c(C#N)c(N2CCC3(CN(c4ccc(OC(F)(F)F)cc4)C3)C2)c2nc(Br)ccc21 ; Cn1c(=O)c(C#N)c(N2CCC3(CN(c4ccc(OC(F)(F)F)cc4)C3)C2)c2nc(C#N)ccc21]

Step 1: To a solution of 4-1 (2.5 g, 10.37 mmol) in dioxane (20 mL) was added Cs₂CO₃ (6.8 g, 20.75 mmol), Pd₂(dba)₃ (0.9 g, 1.04 mmol), BINAP (1.3 g, 2.08 mmol) and tert-butyl 2,6-diazaspiro[3.4]octane-6-carboxylate (2.64 g, 12.45 mmol). The resulting mixture was degassed for 10 minutes, then stirred for 4 h at 120° C. The mixture was filtered and the filtrate was concentrated. The residue was purified by column chromatography on silica gel (petroleum ether/ethyl acetate=1/0 to 3/1) to afford 4-2.

Step 2: To a solution of 4-2 (2.9 g, 7.8 mmol) in dichloromethane (20 mL) was added TFA (5 mL, 805.6 mmol). Then the reaction mixture was stirred at 25° C. for 3 h. The pH was adjusted to 6 by aqueous solution of NaHCO₃ and the mixture was extracted with ethyl acetate. The organic layer was dried over Na₂SO₄, filtered and concentrated to afford 4-3.

Compound 4-4 was prepared from compound 4-3 following the procedure for the synthesis of compound 1-10 in example 1.

Compound 4 was prepared as a 0.33 eq of TFA salt from compound 4-4 following the procedure for the synthesis of compound 1 in example 1. LCMS (ESI, m/z): [M+H]⁺=481.2; ¹H NMR (400 MHz, DMSO-d₆, ppm) δ 8.24-8.22 (m, 1H), 8.08-8.05 (m, 1H), 7.16-7.13 (m, 2H), 6.49-6.47 (m, 2H), 4.40 (s, 2H), 4.24 (t, J=6.7 Hz, 2H), 3.87-3.81 (m, 4H), 3.49 (s, 3H), 2.24 (t, J=6.9 Hz, 2H). ¹⁹F NMR (376 MHz, DMSO-d₆, ppm) 6-57.34 (3F), −73.62 (1F, TFA).

### Example 5 Synthesis of Compound 5

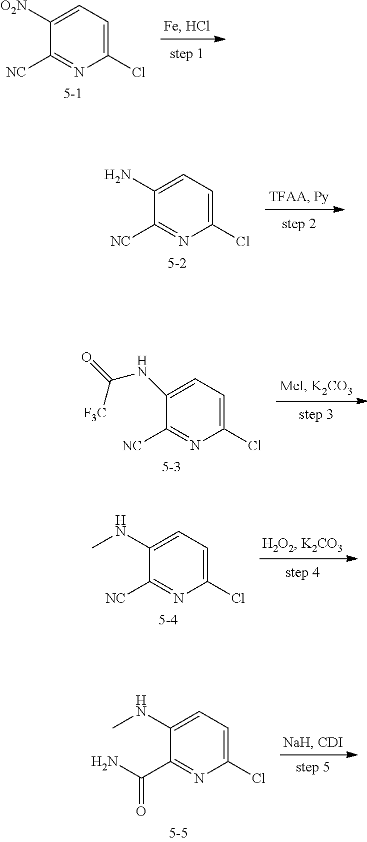 [SMILES: N#Cc1nc(Cl)ccc1[N+](=O)[O-] ; N#Cc1nc(Cl)ccc1N ; N#Cc1nc(Cl)ccc1NC(=O)C(F)(F)F ; CNc1ccc(Cl)nc1C#N ; CNc1ccc(Cl)nc1C(N)=O]

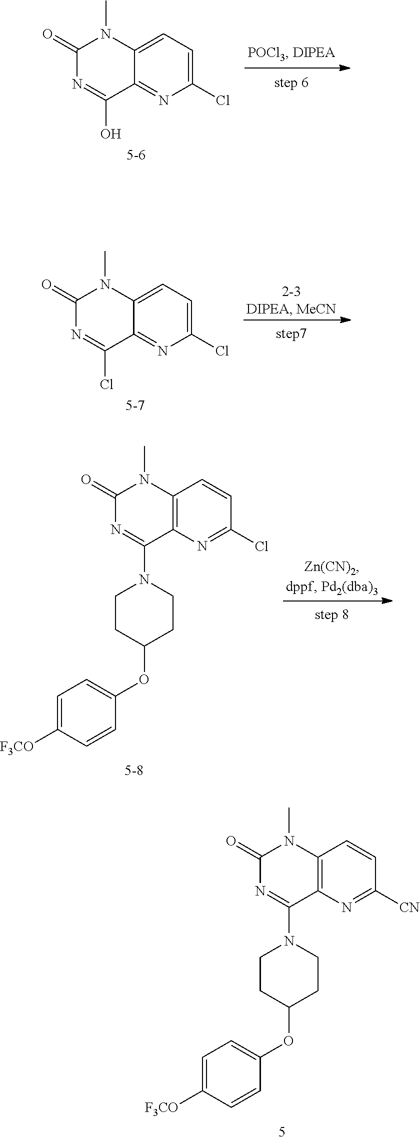 [SMILES: Cn1c(=O)nc(O)c2nc(Cl)ccc21 ; Cn1c(=O)nc(Cl)c2nc(Cl)ccc21 ; "2-3DIPEA, MeCN" ; Cn1c(=O)nc(N2CCC(Oc3ccc(OC(F)(F)F)cc3)CC2)c2nc(Cl)ccc21 ; "Zn(CN)2,dppf, Pd2(dba)3" ; Cn1c(=O)nc(N2CCC(Oc3ccc(OC(F)(F)F)cc3)CC2)c2nc(C#N)ccc21]

Step 1: To a solution of 5-1 (46 g, 250.6 mmol) in con. HCl (170 mL) and EtOH (510 mL) was added Fe powder (42.0 g, 751.8 mmol) at 40° C. in portions. Then the mixture was stirred at 80° C. for 1 h. The mixture was filtered and the filtrate was concentrated. The pH of the mixture was adjusted to 8 with ammonia. Then the mixture was filtered and the filtrate was extracted with ethyl acetate. The combined organic layers were dried over Na₂SO₄, filtered and the filtrate was concentrated to afford 5-2.

Step 2: To a solution of 5-2 (36 g, crude) in THF (400 mL) was added pyridine (56.6 mL, 703.3 mmol), DMAP (2.9 g, 23.4 mmol) and TFAA (73.9 g, 351.6 mmol) at 0° C., slowly. Then the mixture was stirred at room temperature for 0.5 h. The mixture was concentrated. H₂O was added to the residue. The mixture was extracted with ethyl acetate. The combined organic layers were dried over Na₂SO₄, filtered and the filtrate was concentrated. The residue was purified by column chromatography on silica gel (petroleum ether/ethyl acetate=1/0 to 2/1) to afford 5-3.

Step 3: To a solution of 5-3 (47 g, 188.3 mmol) in DMF (450 mL) was added K₂CO₃ (78.1 g, 565 mmol) and CH₃I (35.2 mL, 565 mmol) at room temperature. The mixture was stirred at room temperature for 12 h, then quenched with H₂O. The mixture was extracted with ethyl acetate. The combined organic layers were dried over Na₂SO₄, filtered and the filtrate was concentrated. The residue was purified by column chromatography on silica gel (petroleum ether/ethyl acetate=1/0 to 2/1) to afford 5-4.

Step 4: To a mixture of 5-4 (49.0 g, 175.4 mmol) and K₂CO₃ (72.7 g, 526.3 mmol) in DMSO (500 mL) was added H₂O₂(30%, 59.7 g, 526.3 mmol) at 0° C., slowly. The mixture was stirred at room temperature for 2 h. H₂O was added to the mixture, the mixture was extracted with ethyl acetate. The combined organic layers were dried over Na₂SO₄, filtered and the filtrate was concentrated. The residue was purified by column chromatography on silica gel (petroleum ether/ethyl acetate=1/0 to 2/1) to afford 5-5.

Step 5: To a solution of 5-5 (27 g, 87.3 mmol) in DMF (300 mL) was added NaH (60%, 7.0 g, 174.6 mmol) at 0° C. in portions. After the mixture was stirred at 0° C. for 1 h, CDI (21.2 g, 130.9 mmol) was added to the reaction mixture. Then the mixture was stirred at 70° C. for 2 h. The solid was collected by filtration and washed with ethyl acetate and methanol to afford 5-6.

Step 6: To a solution of 5-6 (1.5 g, 7.09 mmol) in toluene (15 mL) were added POCl₃ (5.27 mL, 56.71 mmol) and DIPEA (2.93 mL, 17.72 mmol) at room temperature. Then the mixture was stirred at 110° C. for 12 h. The mixture was concentrated to afford 5-7 which was used for the next step directly without further purification.

Step 7: To a solution of 5-7 (crude, 1.2 g) and DIPEA (2.5 mL, 15.1 mmol) in acetonitrile (10 mL) was added 2-3 (500 mg, 1.68 mmol) at room temperature. Then the mixture was stirred at 80° C. for 1 h. The mixture was concentrated. The residue was purified by column chromatography on silica gel (petroleum ether/ethyl acetate=1/0 to 1/4) to afford 5-8.

Step 10: To a solution of 5-8 (98 mg, 0.22 mmol) in NMP (6 mL) were added dppf (11.9 mg, 0.022 mmol), Zn (5.6 mg, 0.088 mmol), Zn(CN)₂ (50.6 mg, 0.43 mmol) and Pd₂(dba)₃ (19.7 mg, 0.022 mmol) at room temperature. Then the mixture was stirred at 90° C. for 3 h. The mixture was quenched with H₂O and extracted with ethyl acetate. The combined organic layers were dried over Na₂SO₄, filtered and the filtrate was concentrated under vacuum. The residue was purified by reverse phase HPLC (acetonitrile/0.05% TFA in water: 5%-90%) to afford 5 as a 0.4 eq of TFA salt. LCMS (ESI, m/z): [M+H]⁺=446.4; ¹H NMR (400 MHz, DMSO-d6, ppm): δ 8.26 (d, J=9.2 Hz, 1H), 8.02 (d, J=9.2 Hz, 1H), 7.35-7.26 (m, 2H), 7.18-7.06 (m, 2H), 4.82-4.71 (m, 1H), 3.96-3.79 (m, 4H), 3.46 (s, 3H), 2.20-2.05 (m, 2H), 1.86-1.70 (m, 2H). ¹⁹F NMR (376 MHz, DMSO-d6, ppm): δ −57.23 (3F), −74.35 (1.2F, TFA).

### Example 6 Synthesis of Compound 12

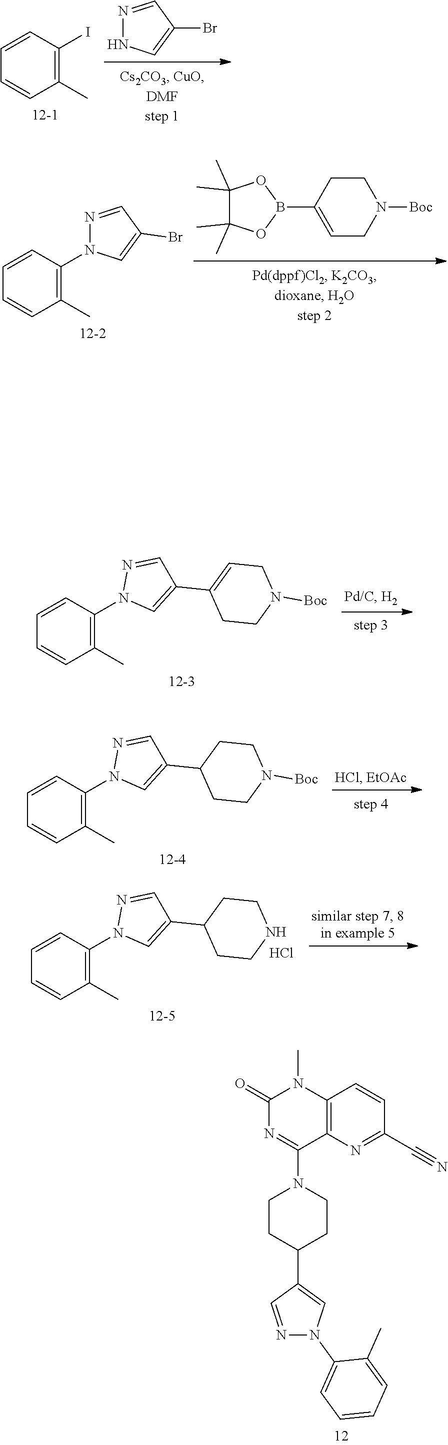

Step 1: To a solution of 12-1 (7.4 g, 34 mmol) in DMSO (100 mL) was added 4-bromo-1H-pyrazole (5.0 g, 34 mmol), K₃PO₄ (21.7 g, 102 mmol), CuO (0.2 g, 1.7 mmol) and N1,N2-bis(furan-2-ylmethyl)oxalamide (0.4 g, 1.7 mmol). The reaction mixture was degassed with nitrogen 3 times, then the reaction mixture was stirred for 14 h at 120° C. The reaction mixture was diluted with water and extracted with ethyl acetate. The combined organic layers were washed with water and brine, dried and concentrated. The residue was purified by column chromatography on silica gel (petroleum ether/ethyl acetate=1/0 to 5/1) to afford 12-2.

Step 2: To a solution of tert-butyl 4-(tetramethyl-1,3,2-dioxaborolan-2-yl)-1,2,3,6-tetrahydropyridine-1-carboxylate (4.7 g, 15.2 mmol) in dioxane (60 mL) was added 12-2 (3 g, 12.7 mmol) and K₂CO₃ (5.2 g, 38 mmol). The reaction mixture was degassed with nitrogen 3 times, then Pd(dppf)Cl₂ (0.9 g, 1.27 mmol) and H₂O (20 mL) were added to the reaction mixture. The reaction mixture was stirred for 14 h at 80° C. The reaction mixture was diluted with water and extracted with ethyl acetate. The combined organic layers were washed with water and brine, dried over Na₂SO₄, and concentrated. The residue was purified by column chromatography on silica gel (petroleum ether/ethyl acetate=1/0 to 5/1) to afford 12-3.

Step 3: To a solution of 12-3 (973 mg, 2.9 mmol) in dichloromethane (10 mL) was added Pd/C (10%, 97.6 mg). The reaction mixture was degassed with H₂ 3 times. The reaction mixture was stirred at room temperature for 14 h under H₂ atmosphere. The reaction mixture was filtered and the filtrate was concentrated. The residue was purified by column chromatography on silica gel (petroleum ether/ethyl acetate=1/0 to 5/1) to afford 12-4.

Step 4: To a solution of 12-4 (826 mg, 2.4 mmol) in dichloromethane (5 mL) was added HCl in ethyl acetate (4M, 5 mL, 20 mmol) and the reaction mixture was stirred at room temperature for 16 h. The reaction mixture was concentrated under reduced pressure to afford 12-5 which was used for the next step directly without further purification.

Compound 12 was prepared from compound 12-5 following the procedure for the synthesis of compound 5 in example 5. LCMS (ESI, m/z): [M+H]⁺=426.2; ¹H NMR (400 MHz, methanol-d₄, ppm) δ 8.11 (d, J=8.9 Hz, 1H), 8.03 (d, J=8.9 Hz, 1H), 7.74 (s, 1H), 7.68 (s, 1H), 7.39-7.36 (m, 2H), 7.33-7.30 (m, 2H), 3.61 (s, 3H), 3.36-3.34 (m, 2H), 3.32-3.29 (m, 2H), 3.17-3.11 (m, 1H), 2.28-2.21 (m, 2H), 2.20 (s, 3H), 1.94-1.81 (m, 21).

### Example 7 Synthesis of Compound 16 and 17

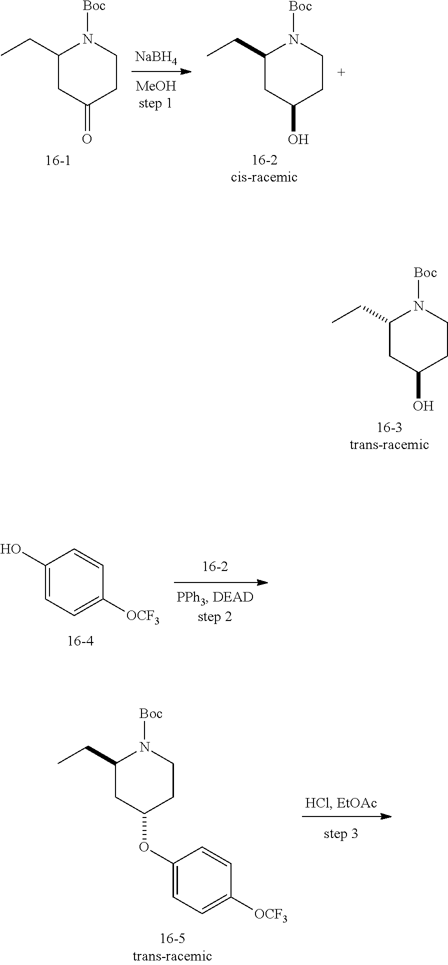

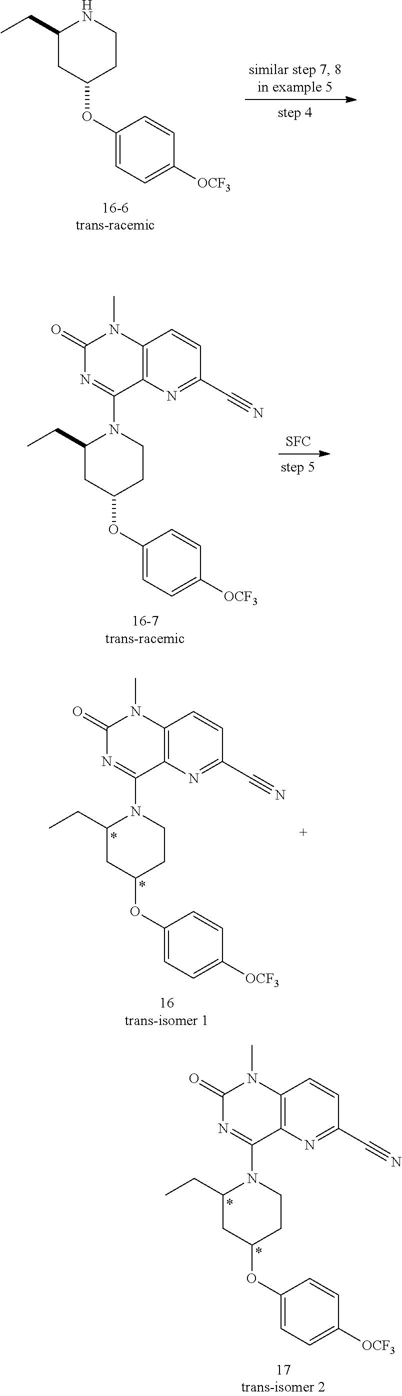

Step 1: To a solution of 16-1 (1.9 g, 8.36 mmol) in methanol (12 mL) was added NaBH₄ (0.2 g, 10.87 mmol) at 0° C. The mixture was stirred at 20° C. for 16 h. The mixture was quenched with water and concentrated. The reside was diluted with H₂O and extracted with ethyl acetate. The combined organic layers were dried over Na₂SO₄, filtered and concentrated. The residue was purified by column chromatography on silica gel (petroleum ether/ethyl acetate=1/0 to 2/1) to afford 16-2 and 16-3.

Step 2: To a solution of 16-2 (604 mg, 2.63 mmol), 4-(trifluoromethoxy)phenol (563 mg, 3.16 mmol) and PPh₃ (1.04 g, 3.95 mmol) in toluene (6 mL) was added DIAD (0.78 mL, 3.95 mmol) at 0° C. under N₂. The mixture was stirred at 110° C. under N₂ for 5 h. The mixture was concentrated. The residue was purified by column chromatography on silica gel (petroleum ether/ethyl acetate=1/0 to 5/1) to afford 16-5.

Step 3: To a solution of 16-5 (923 mg, 2.37 mmol) in dichloromethane (5 mL) was added HCl in ethyl acetate (4M, 2.37 mL, 9.5 mmol) at 25° C. The mixture was stirred at 25° C. for 3 h. The mixture was concentrated to afford 16-6 which was used for the next step directly without further purification.

Step 4: Compound 16-7 was prepared from compound 16-6 following the procedure for the synthesis of compound 5 in example 5.

Step 5: 16-7 (70 mg) was purified by SFC (column: DAICELCHIRALCEL®OZ, methanol (+0.1% 7.0 mol/l Ammonia in methanol)/Supercritical CO₂=40/60) to afford 16 (31.8 mg) and 17 (7.02 mg). respectively. 16: SFC analysis: 99.52% ee; retention time: 1.87 min; column: DAICELCHIRALPAK®OZ, methanol (0.1% DEA) in CO₂, 40%; pressure: 1800 psi; flow rate: 1.5 mL/min. LCMS (ESI, m/z): [M+H]⁺=474.2; ¹H NMR (400 MHz, DMSO-d₆, ppm): δ 8.28-8.23 (m, 1H), 8.04-7.99 (m, 1H), 7.33-7.29 (m, 2H), 7.11-7.07 (m, 2H), 4.85-4.77 (m, 1H), 3.47-3.43 (m, 4H), 3.37-3.29 (m, 2H), 2.17-2.02 (m, 3H), 2.01-1.89 (m, 3H), 0.87-0.79 (m, 3H). ¹⁹F NMR (376 MHz, DMSO-d₆, ppm): δ −57.23 (3F). 17: SFC analysis: 96.6% ee; retention time: 2.14 min; column: DAICELCHIRALPAK®OZ, methanol (0.1% DEA) in CO₂, 40%; pressure: 1800 psi; flow rate: 1.5 mL/min. LCMS (ESI, m/z): [M+H]⁺=474.2; ¹H NMR (400 MHz, DMSO-d₆, ppm): δ 8.23-8.17 (m, 1H), 7.99-7.92 (m, 1H), 7.30-7.22 (m, 2H), 7.10-6.98 (m, 2H), 4.81-4.71 (m, 1H), 3.40 (s, 3H), 3.34-3.32 (m, 3H), 2.13-1.83 (m, 6H), 0.83-0.73 (m, 3H). ¹⁹F NMR (376 MHz, DMSO-d₆, ppm): δ −57.23 (3F).

### Example 8 Synthesis of Compound 21

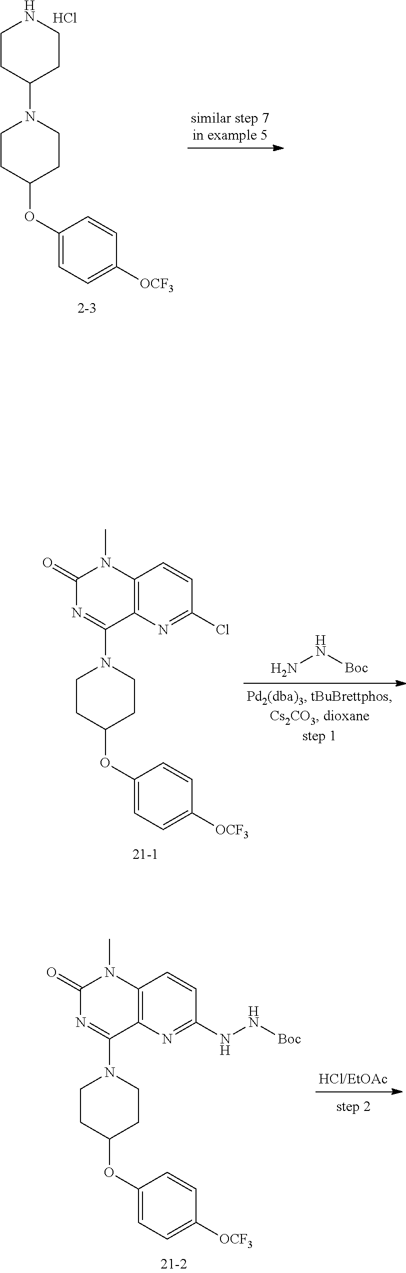 [SMILES: FC(F)(F)Oc1ccc(OC2CCN(C3CCNCC3)CC2)cc1 ; Cl ; Cn1c(=O)nc(N2CCC(Oc3ccc(OC(F)(F)F)cc3)CC2)c2nc(Cl)ccc21 ; CC(C)(C)OC(=O)NN ; Cn1c(=O)nc(N2CCC(Oc3ccc(OC(F)(F)F)cc3)CC2)c2nc(NNC(=O)OC(C)(C)C)ccc21]

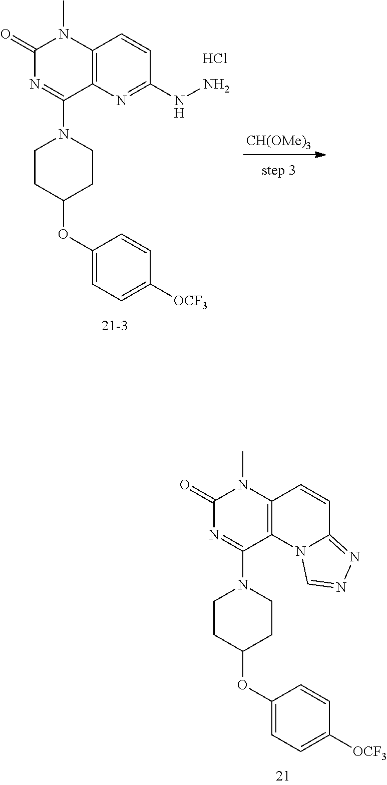

Step 1: Compound 21-1 was prepared from compound 2-3 following the procedure for the synthesis of compound 5-8 in example 5.

Step 2: To a mixture of 21-1 (730 mg, 1.61 mmol) and (tert-butoxy)carbohydrazide (640 mg, 4.8 mmol) in dioxane (10 mL) were added Pd₂(dba)₃ (147.0 mg, 0.16 mmol), Cs₂CO₃ (1.57 g, 4.8 mmol) and t-BuBrettPhos (77.8 mg, 0.16 mmol) at room temperature. Then the mixture was stirred at 80° C. for 1 h. This mixture was filtered and the filtrate was concentrated. The residue was purified by column chromatography on silica gel (dichloromethane/methanol=1/0 to 10/1) to afford 21-2.

Step 3: To a solution of 21-2 (310 mg, 0.56 mmol) in methanol (5 mL) was added HCl in ethyl acetate (4M, 3 mL) at room temperature. Then the mixture was stirred at room temperature for 0.5 h. The mixture was concentrated to afford 21-3.

Step 4: To a solution of 21-3 (250 mg, crude) in methanol (6 mL) was added CH(OCH₃)₃(3 mL, 0.56 mmol) at room temperature. Then the mixture was heated at 100° C. for 12 h. The mixture was concentrated. The residue was purified by column chromatography on silica gel (dichloromethane/methanol=1/0 to 10/1) to afford 21. LCMS (ESI, m/z): [M+H]⁺=461.4; ¹H NMR (400 MHz, DMSO-d₆, ppm) δ 9.49 (d, J=3.3 Hz, 1H), 8.26 (d, J=10.1 Hz, 1H), 7.77 (d, J=10.1 Hz, 1H), 7.33-7.26 (m, 2H), 7.15-7.08 (m, 2H), 4.73-4.65 (m, 1H), 3.68-3.50 (m, 5H), 3.44-3.36 (m, 1H), 3.29-3.22 (m, 1H), 2.17-1.87 (m, 3H), 1.78-1.65 (m, 1H). ¹⁹F NMR (376 MHz, DMSO-d₆, ppm): δ −57.23 (3F).

### Example 9 Synthesis of Compound 23, 24 and 25

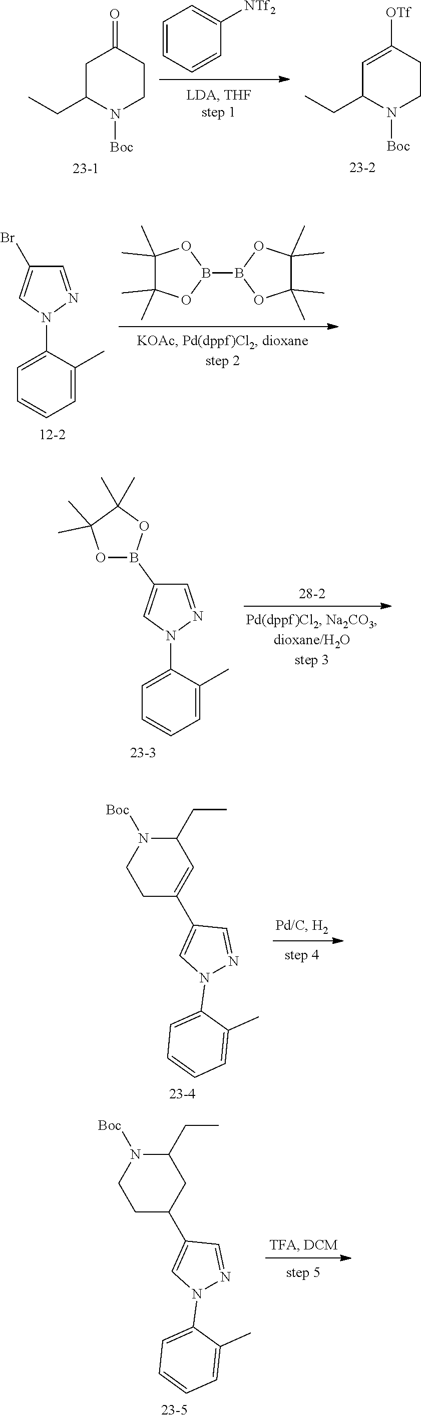

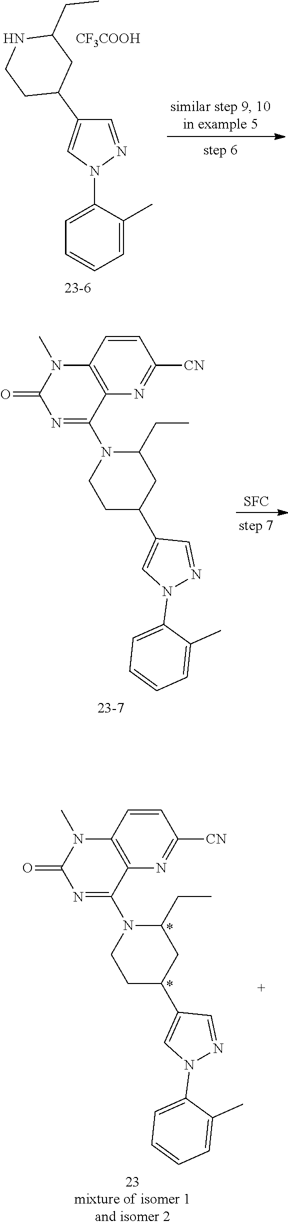 [SMILES: CCC1CC(c2cnn(-c3ccccc3C)c2)CCN1 ; "CF3COOH" ; CCC1CC(c2cnn(-c3ccccc3C)c2)CCN1c1nc(=O)n(C)c2ccc(C#N)nc12 ; "SFC" ; CCC1CC(c2cnn(-c3ccccc3C)c2)CCN1c1nc(=O)n(C)c2ccc(C#N)nc12]

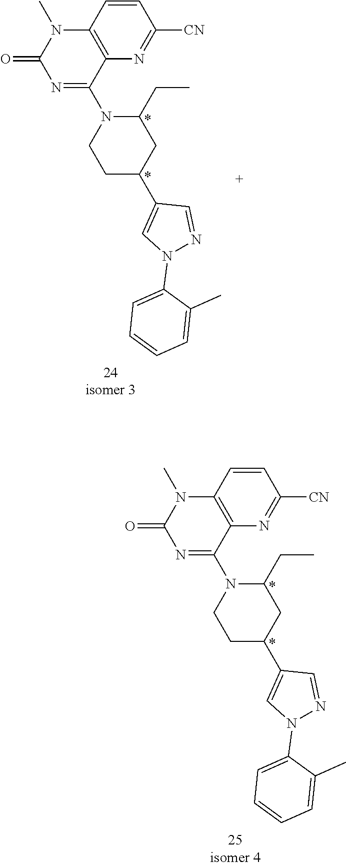 [SMILES: CCC1CC(c2cnn(-c3ccccc3C)c2)CCN1c1nc(=O)n(C)c2ccc(C#N)nc12 ; CCC1CC(c2cnn(-c3ccccc3C)c2)CCN1c1nc(=O)n(C)c2ccc(C#N)nc12]

Step 1: To a solution of LDA (2M in THF, 1.57 mL, 3.15 mmol) in THF (20 mL) was added dropwise 23-1 (550 mg, 2.42 mmol) at −68° C. After 30 minutes, a solution of 1,1,1-trifluoro-N-phenyl-N-trifluoromethanesulfonylmethanesulfonamide (864.44 mg, 2.42 mmol) in THF (5 mL) was added. The reaction mixture was stirred at room temperature for 16 h. The mixture was quenched with saturated NH₄Cl and extracted with ethyl acetate. The combined organic layers were dried over Na₂SO₄, filtered and the filtrate was concentrated. The residue was purified by column chromatography on silica gel (petroleum ether/ethyl acetate=1/0 to 1/1) to afford 23-2.

Step 2: To a solution of 12-2 (1.0 g, 4.22 mmol), 4,4,5,5-tetramethyl-2-(4,4,5,5-tetramethyl-1,3,2-dioxaborolan-2-yl)-1,3,2-dioxaborolane (1.39 g, 5.48 mmol) in dioxane (20 mL) was added KOAc (0.8 g, 8.44 mmol) and Pd(dppf)Cl₂ (0.3 g, 0.42 mmol). The mixture was stirred at 100° C. for 16 h under N₂ atmosphere. The mixture was quenched with H₂O and extracted with ethyl acetate. The combined organic layers were dried over Na₂SO₄, filtered and the filtrate was concentrated. The residue was purified by column chromatography on silica gel (petroleum ether/ethyl acetate=1/0 to 1/1) to afford 23-3.

Step 3: To a solution of 23-2 (500 mg, 1.39 mmol), 23-3 (395.4 mg, 1.39 mmol) in dioxane (20 mL) and H₂O (5 mL) was added Pd(dppf)Cl₂ (113.6 mg, 0.14 mmol) and Na₂CO₃ (295 mg, 2.78 mmol). The mixture was stirred at 90° C. for 16 h under N₂ atmosphere. The mixture was concentrated. The residue was purified by column chromatography on silica gel (petroleum ether/ethyl acetate=1/0 to 1/1) to afford 23-4.

Step 4: To a solution of 23-4 (310 mg, 0.84 mmol) in methanol (10 mL) was added 10% Pd/C (179.5 mg). The mixture was degassed with H₂ several times. Then the mixture was stirred at 25° C. under H₂ atmosphere (1 atm) for 16 h. The mixture was filtered and the filtrate was concentrated to afford 23-5 which was used for the next step directly without further purification.

Step 5: To a solution of 23-5 (240 mg, crude) in dichloromethane (4 mL) was added TFA (1 mL). The mixture was stirred at 25° C. for 1 h. The mixture was concentrated to afford 23-6 which was used for the next step directly without further purification.

Step 6: Compound 23-7 was prepared from compound 23-6 following the procedure for the synthesis of compound 5 in example 5.

Step 7: 23-7 (120 mg, 0.26 mmol) was purified by prep-SFC (column: DAICELCHIRALCEL®AS, methanol (+0.1% 7.0 mol/1 Ammonia in methanol)/Supercritical CO₂=40/60) to afford 23 (25.05 mg), 24 (3.11 mg) and 25 (9.99 mg). 23: SFC analysis: retention time: 1.795 min; column: DAICELCHIRALPAK®AS, methanol (0.1% of DEA) in CO₂, 40%; pressure: 1800 psi; flow rate: 1.5 mL/min; LCMS (ESI, m z): [M+H]⁺=454.4, ¹H NMR (400 MHz, methanol-d₄): δ 8.13-8.06 (m, 1H), 8.06-7.98 (m, 1H), 7.76-7.69 (m, 1H), 7.69-7.63 (m, 1H), 7.39-7.33 (m, 2H), 7.33-7.25 (m, 2H), 6.12-5.00 (m, 2H), 3.72-3.35 (m, 4H), 3.00-2.48 (m, 1H), 2.35-1.69 (m, 9H), 1.16-0.94 (m, 3H). 24: SFC analysis: 99.76% de; retention time: 2.300 min; column: DAICELCHIRALPAK®AS, methanol (0.1% of DEA) in CO₂, 40%; pressure: 1800 psi; flow rate: 1.5 mL/min; LCMS (ESI, m/z): [M+H]⁺=454.4, ¹H NMR (400 MHz, methanol-d₄): δ 8.16-8.05 (m, 1H), 8.05-7.96 (m, 1H), 7.70 (s, 1H), 7.65 (s, 1H), 7.39-7.33 (m, 2H), 7.33-7.25 (m, 2H), 6.05-5.03 (m, 2H), 3.58 (s, 3H), 3.36 (s, 1H), 2.28-2.10 (m, 6H), 2.11-1.99 (m, 1H), 1.95-1.86 (m, 1H), 1.85-1.72 (m, 1H), 1.36-1.27 (m, 1H), 1.11-0.97 (m, 3H). 25: SFC analysis: 100% de; retention time: 2.422 min; column: DAICELCHIRALPAK®AS, methanol (0.1% of DEA) in CO₂, 40%; pressure: 1800 psi; flow rate: 1.5 mL/min; LCMS (ESI, m/z): [M+H]⁺=454.4, ¹H NMR (400 MHz, methanol-d₄): δ 8.19-8.06 (m, 1H), 8.05-7.96 (m, 1H), 7.75-7.70 (m, 1H), 7.67 (s, 1H), 7.40-7.34 (m, 2H), 7.34-7.27 (m, 2H), 5.17 (s, 2H), 3.61-3.55 (m, 3H), 3.55-3.44 (m, 1H), 3.00-2.81 (m, 1H), 2.65-2.51 (m, 1H), 2.36-2.22 (m, 1H), 2.22-2.16 (m, 3H), 2.14-1.98 (m, 1H), 2.01-1.85 (m, 2H), 1.78 (s, 1H), 1.40-1.26 (m, 1H), 1.13-0.97 (m, 3H).

### Example 10 Synthesis of Compound 26 and 27

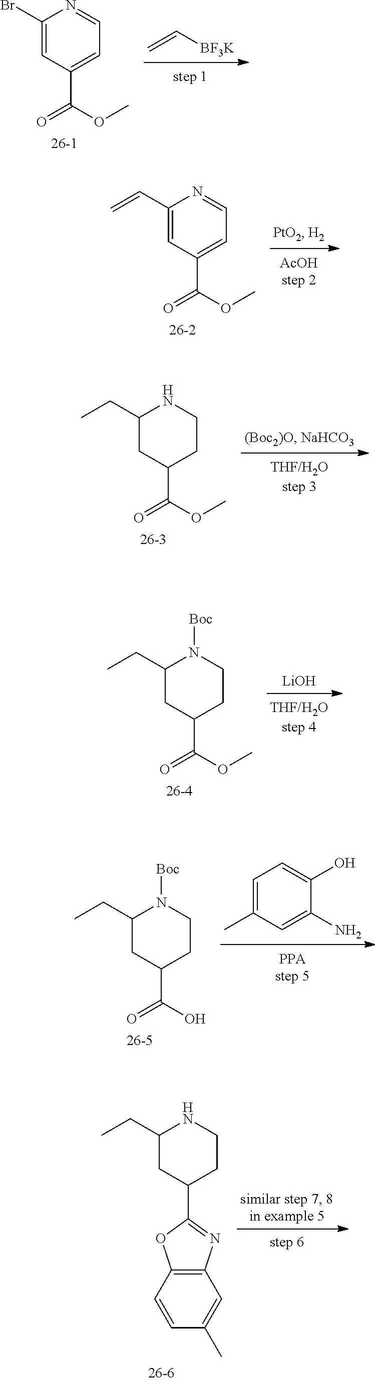

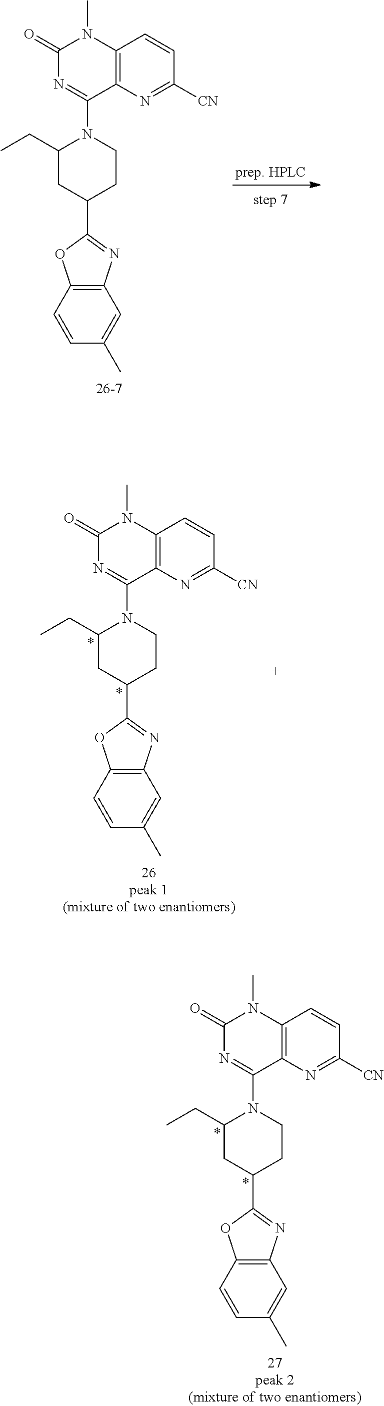 [SMILES: CCC1CC(c2nc3cc(C)ccc3o2)CCN1c1nc(=O)n(C)c2ccc(C#N)nc12 ; CCC1CC(c2nc3cc(C)ccc3o2)CCN1c1nc(=O)n(C)c2ccc(C#N)nc12 ; CCC1CC(c2nc3cc(C)ccc3o2)CCN1c1nc(=O)n(C)c2ccc(C#N)nc12]

Step 1: To a mixture of 26-1 (10 g, 46.29 mmol) and potassium trifluoro(vinyl)borate (7.44 g, 55.55 mmol) in THF (160 mL) and H₂O (40 mL) was added 1,1′-bis(di-t-butylphosphino)ferrocene palladium dichloride (3.0 g, 4.63 mmol) and K₃PO₄ (24.6 g, 115.73 mmol) under N₂. Then the mixture was stirred at room temperature for 16 h. The mixture quenched by addition of water. The aqueous layer was extracted with ethyl acetate. The organic layers were combined, washed with brine, dried over anhydrous Na₂SO₄ and concentrated. The residue was purified by column chromatography on silica gel (petroleum ether/ethyl acetate=5/1) to afford 26-2.

Step 2: To a mixture of methyl 26-2 (7.8 g, 47.8 mmol) in AcOH (100 mL) was added PtO₂ (2.2 g, 9.56 mmol). The mixture was degassed with H₂ several times, then the mixture was stirred at 80° C. for 16 h under H₂ (1 atm) atmosphere. The mixture was filtered and the filtrate was concentrated to afford 26-3 which was used for the next step directly without further purification.

Step 3: To a mixture of 26-3 (8 g, 46.72 mmol) in THF (80 mL) and H₂O (80 mL) was added NaHCO₃ (39.2 g, 467.18 mmol) and (BoC)₂O (21.47 mL, 93.44 mmol). Then the mixture was stirred at room temperature for 3 h. The mixture quenched by addition of water, and the aqueous layer was extracted with ethyl acetate. The organic layers were combined, washed with brine, dried over anhydrous Na₂SO₄ and concentrated. The residue was purified by column chromatography on silica gel (petroleum ether/ethyl acetate=5/1) to afford 26-4.

Step 4: To a mixture of 26-4 (9.6 g, 35.38 mmol) in THF (60 mL) and H₂O (60 mL) was added LiGH (7.4 g, 176.89 mmol). Then the mixture was stirred at room temperature for 16 h. The pH of mixture was adjusted to 4 with ¹N HCl and the aqueous layer was extracted with ethyl acetate. The organic extracts were combined, washed with brine, dried over anhydrous Na₂SO₄ and concentrated to afford 26-5 which was used for the next step directly without further purification.

Step 5: A mixture of 26-5 (4.55 g, crude) and PPA (100 mL) was stirred at 180° C. for 2 h. After being cooled to 90° C., the reaction was quenched by water. The filtrate was adjusted pH to about 12 with 50% potassium hydroxide aqueous solution and then extracted with methylene chloride. The organic layer was dried by Na₂SO₄, filtered and concentrated to afford 26-6 which was used for the next step directly without further purification.

Step 6: Compound 26-7 was prepared from compound 26-6 following the procedure for the synthesis of compound 5 in example 5.

Step 7: 26-7 (150 mg, 0.35 mmol) was separated by reverse phase HPLC (acetonitrile/H₂O, 5-95%) to afford 26 (24.47 mg) and 27 (54.71 mg). 26: Analytical HPLC: retention time: 1.85 min; column: Waters ACQUITY BEH C18 2.1*50 mm, 1.7 um; Mobile phaseA: H₂O (0.05% TFA); Mobile phaseB: Acetonitrile (0.05% TFA); flow rate: 1.0 mL/min; Run time: 5 min; 95 to 5% A in 3 min, 5% A for 2 min. LCMS (ESI, m/z): [M+H]⁺=429.4; ¹H NMR (400 MHz, DMSO-d₆, ppm): δ 8.27-8.23 (m, 1H), 8.01 (d, J=9.2 Hz, 1H), 7.54 (d, J=8.4 Hz, 1H), 7.47 (s, 1H), 7.18-7.15 (m, 1H), 5.77-5.44 (m, 1H), 5.34-4.80 (m, 1H), 3.68-3.61 (m, 2H), 3.45 (s, 3H), 2.40 (s, 3H), 2.29-2.16 (m, 2H), 2.05-1.78 (m, 4H), 1.00-0.89 (m, 3H). 27: Analytical HPLC: retention time: 1.85 min; column: Waters ACQUITY BEH C18 2.1*50 mm, 1.7 um; Mobile phaseA: H₂O (0.05% TFA); Mobile phaseB: Acetonitrile (0.05% TFA); flow rate: 1.0 mL/min; Run time: 5 min; 95 to 5% A in 3 min, 5% A for 2 min. LCMS (ESI, m/z): [M+H]⁺=429.4; ¹H NMR (400 MHz, DMSO-d₆, ppm): δ 8.26 (d, J=8.8 Hz, 1H), 8.00 (d, J=9.2 Hz, 1H), 7.57 (d, J=8.4 Hz, 1H), 7.47 (s, 1H), 7.18-7.15 (m, 1H), 5.37-4.81 (m, 2H), 3.49-3.46 (m, 2H), 3.45 (s, 3H), 2.42 (s, 3H), 2.37-2.25 (m, 3H), 2.24-2.15 (m, 1H), 1.79-1.64 (m, 1H), 1.53-1.45 (m, 1H), 0.92-0.81 (m, 3H).

### Example 11 Synthesis of Compound 28 and 29

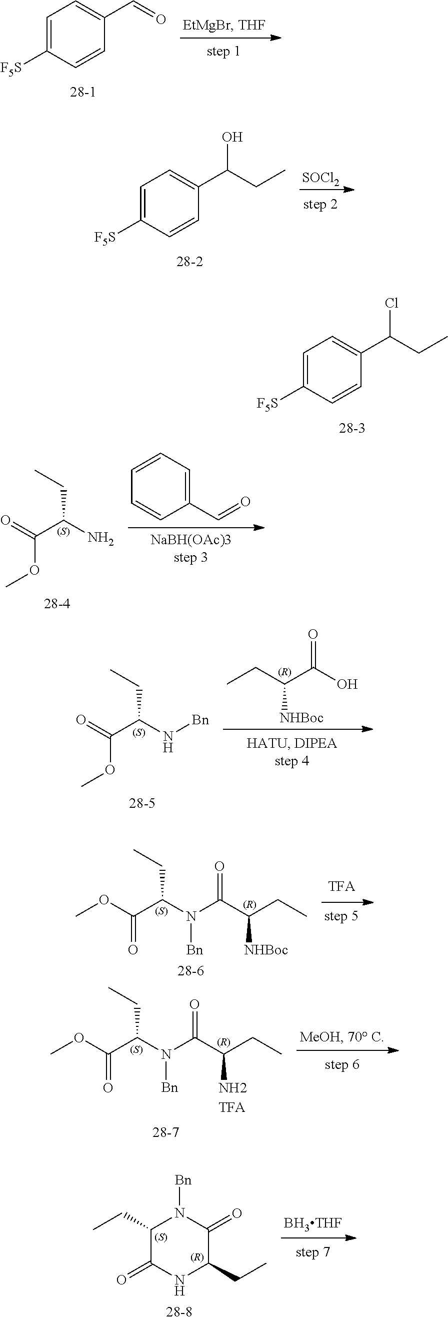

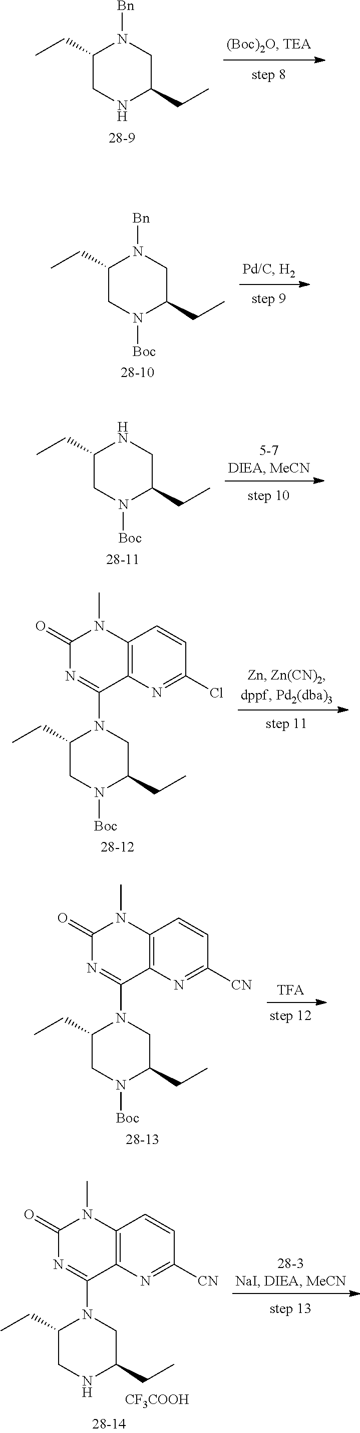

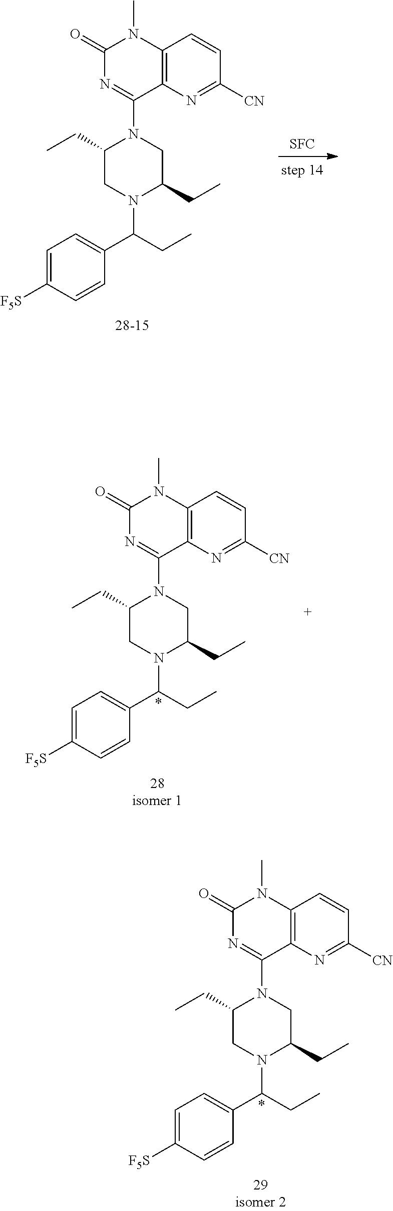

Step 1: A solution of 28-1 (4.5 g, 19.38 mmol) in THF (30 mL) at −30° C. was added C₂H5MgBr (29.07 mL, 29.07 mmol, 1M in THF). The mixture was stirred at room temperature for 2 h. The reaction was quenched with saturated aqueous ammonium chloride. The organic phase was separated, dried over Na₂SO₄, filtered and concentrated. The residue was purified by column chromatography on silica gel (petroleum ether/ethyl acetate=4/1) to afford 28-2.

Step 2: To a solution of 28-2 (1 g, 3.81 mmol) in dichloromethane (10 mL) at 0° C. was added DMF (0.029 mL, 0.38 mmol) and SOCl₂ (0.42 mL, 5.72 mmol). The reaction was stirred at room temperature for 18 h. The reaction mixture was concentrated, and the residue was purified by column chromatography on silica gel (petroleum ether) to afford 28-3.

Step 3: To a stirred solution of 28-4 (100 g, 651 mmol) in dry CH₂Cl₂(1.5 L) were added benzaldehyde (67.5 mL, 664 mmol), K₂CO₃ (90.0 g, 651 mmol) and Na₂SO₄ (92.5 g, 651 mmol). The reaction mixture was stirred at room temperature for 16 h. The reaction mixture was cooled with an ice water bath and NaBH(OAc)₃ (207.0 g, 976 mmol) was added in portions over 30 minutes. Then the mixture was stirred at room temperature for 16 h. The solid was filtered and the filtration was concentrated. The residue was washed with 1 N HCl and extracted with ethyl acetate. The aqueous layer was adjusted to pH=8.0 with NaHCO₃. Then the mixture was extracted with ethyl acetate. The combined organic layers were washed with brine, dried over anhydrous Na₂SO₄, filtered and concentrated to afford 28-5 which was used in the next step directly without further purification.

Step 4: To a solution of (2R)-2-{[(tert-butoxy)carbonyl]amino}butanoic acid (124 g, crude) in DMF (1 L) were added DIPEA (137 mL, 832 mmol) and HATU (253 g, 666 mmol). The reaction was stirred at 0° C. for 10 minutes, then 28-5 (115 g, 555 mmol) was added to the mixture. The resulting mixture was stirred for 16 h at room temperature. The reaction mixture was diluted with ethyl acetate and washed with water. The organic layer was dried over anhydrous Na₂SO₄, filtered and evaporated to afford 28-6 which was used for next step directly without further purification.

Step 5: To a solution of 28-6 (300 g, crude) in dichloromethane (1 L) was added TFA (500 mL, 764 mmol), the reaction was stirred at room temperature for 16 h. The solvent was removed to afford 28-7 which was used for next step directly without further purification.

Step 6: 28-7 (500 g, crude) was dissolved in methanol (1.5 L) and the reaction mixture was heated at 70° C. for 16 h. The solvent was removed, the residue was dissolved in dichloromethane and washed with saturated aqueous NaHCO₃ solution. The combined organic layer was dried over anhydrous Na₂SO₄, filtered and concentrated. The residue was dissolved in propan-2-ol (500 mL) and heated at 70° C. for 1 h. The mixture was filtered, the solution was cooled to −10° C., the solid was filtered and dried to afford 28-8.

Step 7: To a stirred solution of 28-8 (20 g, 76.82 mmol) in dry THF (500 mL) was slowly added BH₃·THF (1M, 768 mL, 768 mmol) at 0° C. The reaction mixture heated at 70° C. for 16 h. The reaction was quenched with methanol and 1.5 N HCl. The solvent was removed, the residue was dissolved in dichloromethane and washed with saturated aqueous NaHCO₃ solution. The organic layer was dried over anhydrous Na₂SO₄, filtered and concentrated to afford 28-9 which was used in the next step directly.

Step 8: To a stirred solution of 28-9 (23 g, crude) in THF (100 mL) and H₂O (100 mL) was added NaHCO₃ (16.6 g, 198 mmol) and (Boc)₂O (34.1 mL, 148.5 mmol). The reaction mixture was stirred at room temperature for 4 h. The mixture was diluted with ethyl acetate and washed with water and brine, dried over anhydrous Na₂SO₄ and concentrated. The residue was purified by column chromatography on silica gel (petroleum ether/ethyl acetate=20/1 to 10/1) to afford 28-10.

Step 9: To a stirred solution of 28-10 (20 g, 60.15 mmol) in ethanol (200 mL) was added Pd/C (10%, 10 g). The reaction mixture was stirred under H₂ atmosphere for 18 h at room temperature. The reaction mixture was filtered, the solvent was removed. The residue was purified by column chromatography on silica gel (dichloromethane/methanol=1/0 to 10/1) to afford 28-11.

Step 10: To a solution of 5-7 (1.63 g, crude) and DIPEA (7 mL, 42.5 mmol) in acetonitrile (40 mL) was added 28-11 (1.37 g, 5.66 mmol) at room temperature and the mixture was stirred at 80° C. for 1 h. Then the mixture was concentrated, the residue was purified by column chromatography on silica gel (petroleum ether/ethyl acetate=1/0 to 1/4) to afford 28-12.

Step 11: To a solution of 28-12 (800 mg, 1.84 mmol) in NMP (8 mL) was added dppf (207.2 mg, 0.37 mmol), Zn (120.0 mg, 1.84 mmol), Zn(CN)₂ (431.0 mg, 3.67 mmol) and Pd₂(dba)₃ (168.0 mg, 0.18 mmol) at room temperature. Then the mixture was heated at 90° C. for 12 h. The mixture was quenched with H₂O and extracted with ethyl acetate. The combined organic layers were dried over Na₂SO₄, filtered and the filtrate was concentrated. The residue was purified by column chromatography on silica gel (petroleum ether/ethyl acetate=1/0 to 1/4) to afford 28-13.

Step 12: To a solution of 28-13 (570 mg, 1.34 mmol) in dichloromethane (5 mL) was added TFA (2 mL, 1.34 mmol) at room temperature. Then the mixture was stirred at room temperature for 1 h. The mixture was concentrated to 28-14 which was used in the next step directly without further purification.

Step 13: The mixture of 28-14 (220 mg, 0.67 mmol), 28-3 (283.8 mg, 1.01 mmol), DIPEA (1.11 mL, 6.74 mmol) and NaI (151.5 mg, 1.01 mmol) in acetonitrile (10 mL) was stirred at 90° C. for 24 h. The solvent was removed, and the residue was purified by Reverse phase HPLC (acetonitrile with 0.05% of TFA in water: 10% to 70%) to afford 28-15.

Step 4: 28-15 (170 mg) was purified by SFC (column: DAICELCHIRALPAK®IG, methanol (+0.1% 7.0 mol/1 Ammonia in methanol)/Supercritical CO₂=40/60) to afford 28 (28 mg) and 29 (18 mg) respectively. 28: SFC analysis: 96.44% de; retention time: 4.29 min; column: DAICELCHIRALPAK®IB, methanol (0.1% of DEA) in CO₂, 5% to 40%; pressure: 1800 psi; flow rate: 1.5 mL/min. LCMS (ESI, m/z): [M+H]⁺=571.5. ¹H NMR (400 MHz, DMSO-d₆, ppm) δ 8.23 (d, J=8.32 Hz, 1H), 7.98 (d, J=8.86 Hz, 1H), 7.89 (d, J=8.52 Hz, 2H), 7.56 (d, J=6.04 Hz, 2H), 5.85-5.47 (m, 1H), 4.97-4.85 (m, 1H), 3.71-3.62 (m, 1H), 3.66 (s, 3H), 3.09 (d, J=12.24 Hz, 1H), 2.93-2.76 (m, 2H), 2.44-2.24 (m, 1H), 2.22-2.06 (m, 1H), 2.05-1.80 (m, 3H), 1.62-1.54 (m, 1H), 1.53-1.39 (m, 1H), 0.97-0.91 (m, 3H), 0.70-0.52 (m, 6H); ¹⁹F NMR (376 MHz, DMSO-d₆, ppm): δ 87.99 (1F), 64.42 (4F) 29: SFC analysis: 97.86% de; retention time: 4.44 min; column: DAICELCHIRALPAK®IB, methanol (0.1% of DEA) in CO₂, 5% to 40%; pressure: 1800 psi; flow rate: 1.5 mL/min. LCMS (ESI, m/z): [M+H]⁺=571.5. ¹H NMR (400 MHz, DMSO-d₆, ppm): δ 8.27-8.19 (m, 1H), 8.01-7.95 (m, 1H), 7.87 (d, J=7.88 Hz, 2H), 7.58 (d, J=8.28 Hz, 2H), 6.05-5.28 (m, 1H), 5.09-4.75 (m, 1H), 3.64-3.50 (m, 2H), 3.43 (s, 3H), 3.18-3.07 (m, 1H), 2.21-2.12 (m, 1H), 2.04-1.95 (m, 1H), 1.83-1.39 (m, 6H), 1.00-0.91 (m, 3H), 0.65-0.54 (m, 6H). ¹⁹F NMR (376 MHz, DMSO-d₆, ppm): δ 88.00 (1F), 64.37 (4F).

### Example 12 Synthesis of Compound 30

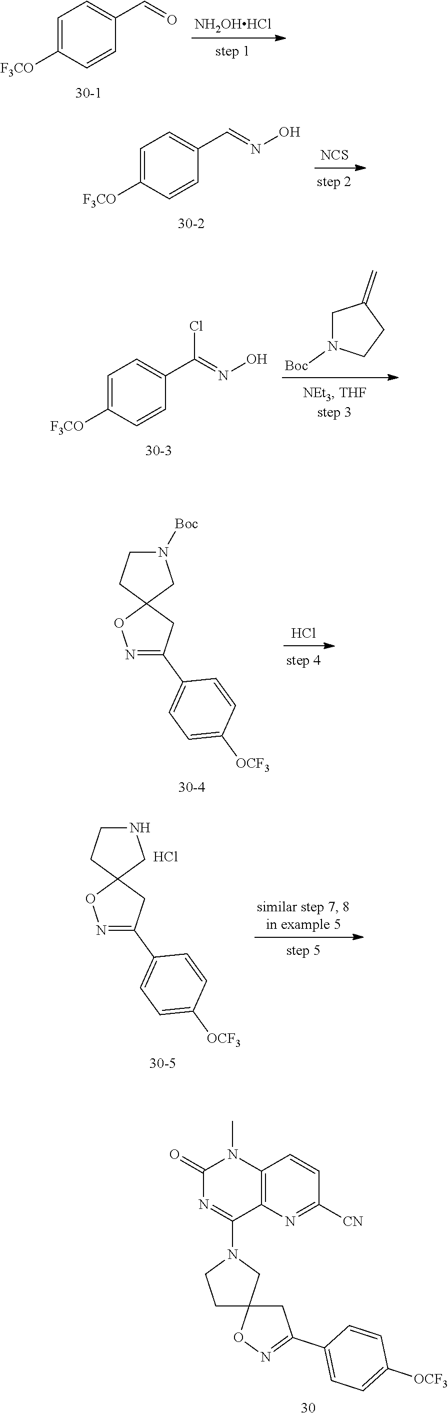

Step 1: To a solution of 30-1 (5 g, 26.3 mmol) and NaOH (1.6 g, 39.5 mmol) in EtOH (30 mL) and H₂O (15 mL) was added hydroxylamine hydrochloride (2.2 g, 31.6 mmol) at 25° C. The mixture was stirred at 25° C. for 16 h. Ice was added to the reaction mixture, the solid was filtered, the filter cake was washed with H₂O and dried to afford 30-2.

Step 2: A solution of 30-2 (1.6 g, 7.8 mmol) and NCS (1.1 g, 8.6 mmol) in DMF (15 mL) was stirred at 25° C. for 16 h. The mixture was concentrated, the residue was diluted with ethyl acetate, washed with H₂O. The organic layer was dried over Na₂SO₄, filtered and concentrated to afford 30-3.

Step 3: To a solution of 30-3 (800 mg, 3.3 mmol) and tert-butyl 3-ethylidenepyrrolidine-1-carboxylate (795.5 mg, 4.3 mmol) in THF (20 mL) was added triethylamine (0.93 mL, 6.7 mmol) at 25° C. The mixture was stirred at 25° C. for 4 h. The mixture was concentrated, the residue was purified by column chromatography on silica gel (petroleum ether/ethyl acetate=1/0 to 4/1) to afford 30-4.

Step 4: To a solution of 30-4 (300 mg, 0.78 mmol) in dichloromethane (5 mL) was added HCl (4 M in ethyl acetate, 2.0 mL) at 25° C. The mixture was stirred at 25° C. for 2 h. The mixture was concentrated to afford 30-5.

Step 5: Compound 30 was prepared from compound 30-4 following the procedure for the synthesis of compound 5 in example 5. LCMS (ESI, m/z): [M+H]⁺=471.2; ¹H NMR (400 MHz, DMSO-d₆, ppm): δ 8.33-8.20 (m, 1H), 8.06-7.94 (m, 1H), 7.88-7.76 (m, 2H), 7.55-7.43 (m, 2H), 4.69-4.49 (m, 1H), 4.44-4.19 (m, 1H), 4.12-3.93 (m, 1H), 3.90-3.68 (m, 2H), 3.64-3.52 (m, 1H), 3.46 (s, 3H), 2.45-2.15 (m, 2H). ¹⁹F NMR (376 MHz, DMSO-d₆, ppm): δ −56.74 (3F).

### Example 13 Synthesis of Compound 32 and 33

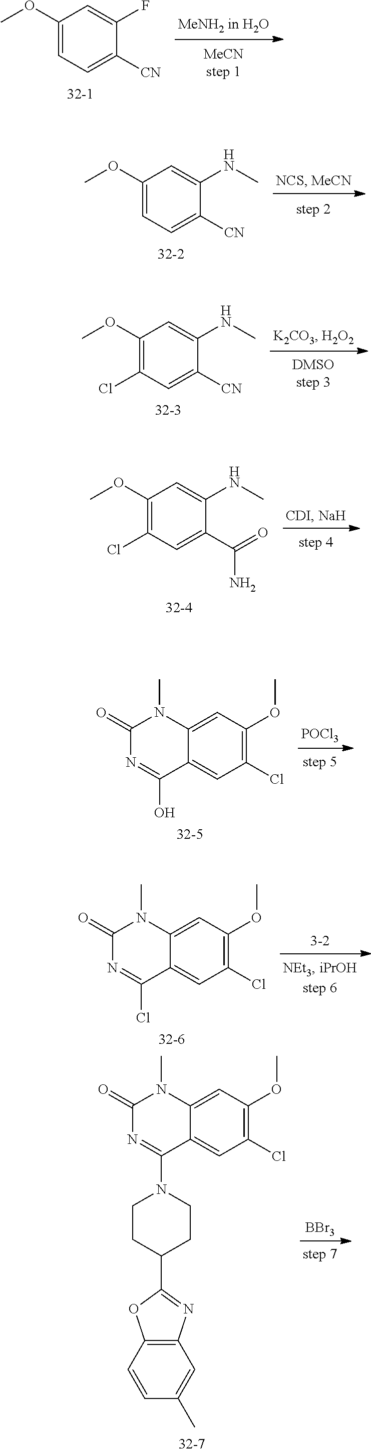

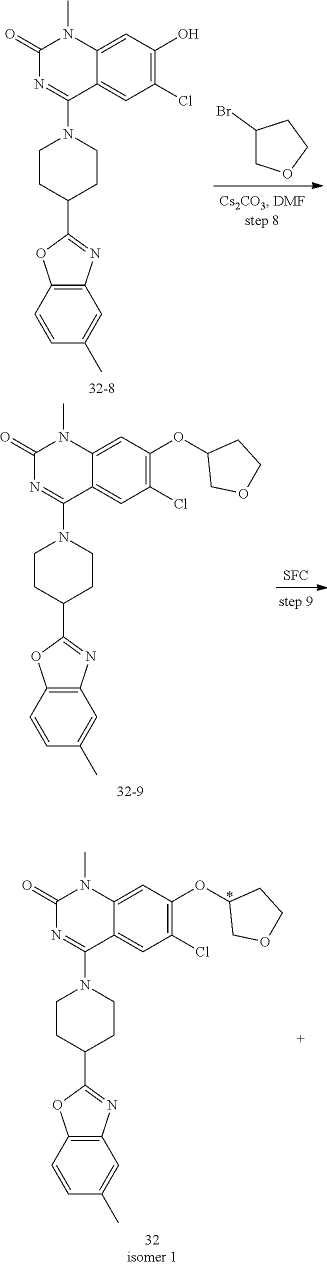 [SMILES: Cc1ccc2oc(C3CCN(c4nc(=O)n(C)c5cc(O)c(Cl)cc45)CC3)nc2c1 ; BrC1CCOC1 ; Cc1ccc2oc(C3CCN(c4nc(=O)n(C)c5cc(OC6CCOC6)c(Cl)cc45)CC3)nc2c1 ; "SFC" ; Cc1ccc2oc(C3CCN(c4nc(=O)n(C)c5cc(OC6CCOC6)c(Cl)cc45)CC3)nc2c1]

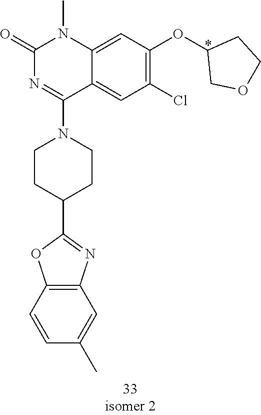 [SMILES: Cc1ccc2oc(C3CCN(c4nc(=O)n(C)c5cc(OC6CCOC6)c(Cl)cc45)CC3)nc2c1]

Step 1: To a solution of 32-1 (15 g, 99.25 mmol) in acetonitrile (100 mL) was added CH₃NH₂ (200 mL, 3.56 mmol, 40% in H₂O). Then the reaction mixture was stirred at 80° C. for 16 h. The reaction mixture was quenched with H₂O. The mixture was extracted with ethyl acetate. The combined organic layers were washed with H₂O. The organic layer was dried over Na₂SO₄, filtered and concentrated to afford 32-2.

Step 2: To a solution of 32-2 (8.2 g, 50.56 mmol) in acetonitrile (150 mL) was added NCS (7.4 g, 55.61 mmol), then the reaction mixture was stirred at 60° C. for 70 minutes. The mixture was quenched by addition of water. The aqueous layer was extracted with ethyl acetate. The organic layer was combined, washed with brine, dried over anhydrous Na₂SO₄, filtered and concentrated. The residue was purified by column chromatography on silica gel (petroleum ether/ethyl acetate=1/0 to 5/1) to afford 32-3.

Step 3: To a solution of 32-3 (5.0 g, 25.43 mmol) in DMSO (60 mL) were added K₂CO₃ (5.3 g, 38.14 mmol) and H₂O₂(17.3 g, 30%, 152.6 mmol). Then the reaction mixture was stirred at 25° C. for 16 h. The reaction mixture was quenched with H₂O. The mixture was extracted with ethyl acetate. The combined organic layers were washed with H₂O. The organic layer was dried over Na₂SO₄, filtered and the filtrate was concentrated to afford 32-4.

Step 4: To a stirred 0° C., solution of 32-4 (5 g, 23.29 mmol) in DMF (70 mL) was in portions added NaH (1.9 g, 46.59 mmol). After stirred at 0° C. for 0.5 h, CDI (5.7 g, 34.94 mmol) was added to the mixture, then the mixture was stirred at 60° C. for 16 h. The reaction mixture was quenched with H₂O. The mixture was extracted with ethyl acetate. The combined organic layer was washed with H₂O. The organic layer was dried by Na₂SO₄, filtered and the filtrate was concentrated to afford 32-5.

Step 5: To a solution of 32-5 (1 g, 4.16 mmol) in toluene (10 mL) was added DIPEA (1.2 g, 9.14 mmol) and POCl₃ (3.2 g, 20.78 mmol). The mixture was stirred at 90° C. for 3 h. The reaction mixture was concentrated to afford 32-6.

Step 6: A mixture of 32-6 (1.0 g, 3.86 mmol), 3-2 (1.00 g, 4.63 mmol) and DIPEA (7.5 g, 57.9 mmol) in propan-2-ol (15 mL) was stirred at 90° C. for 5 h. The mixture was cooled to room temperature. The precipitate was collected by filtration to afford 32-7.

Step 7: To a solution of 32-7 (1.0 g, 2.28 mmol) in dichloromethane (10 mL) under N₂ at −78° C. was added dropwise BBr₃ (5.7 g, 22.8 mmol). After the addition was completed, the mixture was stirred at room temperature for 16 h. Ice water was added to the reaction mixture and the precipitate was collected by filtration, washed with H₂O and dried to afford 32-8.

Step 8: To a mixture of 32-8 (300 mg, 0.71 mmol) in DMF (10 mL) were added Cs₂CO₃ (690 mg, 2.12 mmol) and 3-bromooxolane (213.2 mg, 1.41 mmol). Then the mixture was stirred at 85° C. for 1 h. The solvent was removed under vacuum. The residue was purified by reverse phase HPLC (acetonitrile/H₂O: 5%˜42%) to afford 32-9.

Step 9: 32-9 (100 mg) was purified by SFC (column: DAICELCHIRALPAK®IC, ETOH (+0.1% 7.0 mol/l Ammonia in methanol) to afford 32 (14.80 mg) and 33 (15.23 mg) respectively. 32: SFC analysis: 100% ee; retention time: 9.164 min; column: DAICELCHIRALPAK®IC, ETOH (+0.1% 7.0 mol/l Ammonia in methanol) in CO₂, 40% to 60%; pressure: 100 bar; flow rate: 1.0 mL/min; LCMS (ESI, m/z): [M+H]⁺=495.4; ¹H NMR (400 MHz, DMSO-d₆, ppm): δ 7.79 (s, 1H), 7.56 (d, J=8.0 Hz, 1H), 7.50 (s, 1H), 7.20-7.16 (d, J=8.0 Hz 1H), 6.91 (s, 1H), 5.44-5.42 (m, 1H), 4.22-4.15 (m, 2H), 4.05-3.95 (m, 1H), 3.95-3.78 (m, 3H), 3.51-3.49 (m, 3H), 3.49-3.38 (m, 2H), 2.41 (s, 3H), 2.39-2.30 (m, 2H), 2.22-2.18 (m, 2H), 2.09-1.94 (m, 3H). 33: SFC analysis: 98.89% ee; retention time: 10.858 min; column: DAICELCHIRALPAK®IC, ETOH (+0.1% 7.0 mol/l Ammonia in methanol) in CO₂, 40% to 60%; pressure: 100 bar; flow rate: 1.0 mL/min; LCMS (ESI, m/z): [M+H]⁺=495.4; ¹H NMR (400 MHz, DMSO-d₆, ppm): δ 7.79 (s, 1H), 7.56 (d, J=8.0 Hz, 1H), 7.50 (s, 1H), 7.20-7.16 (d, J=8.0 Hz 1H), 6.91 (s, 1H), 5.44-5.41 (m, 1H), 4.23-4.15 (m, 2H), 4.01-3.95 (m, 1H), 3.91-3.86 (m, 2H), 3.83-3.78 (m, 1H), 3.51-3.49 (m, 3H), 3.41-3.95 (m, 2H), 2.41 (s, 3H), 2.39-2.30 (m, 2H), 2.22-2.18 (m, 2H), 2.07-1.96 (m, 3H).

### Example 14 Synthesis of Compound 34 and 35

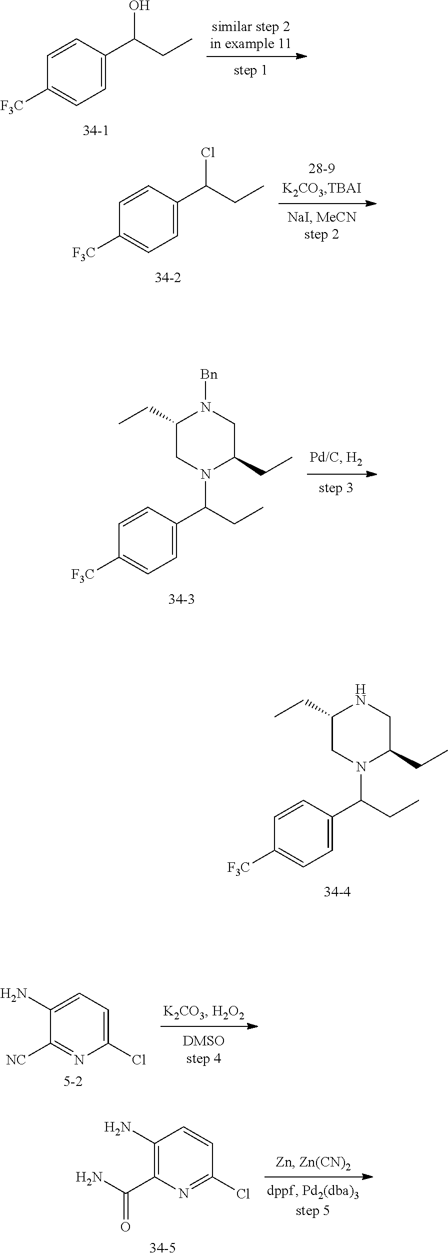

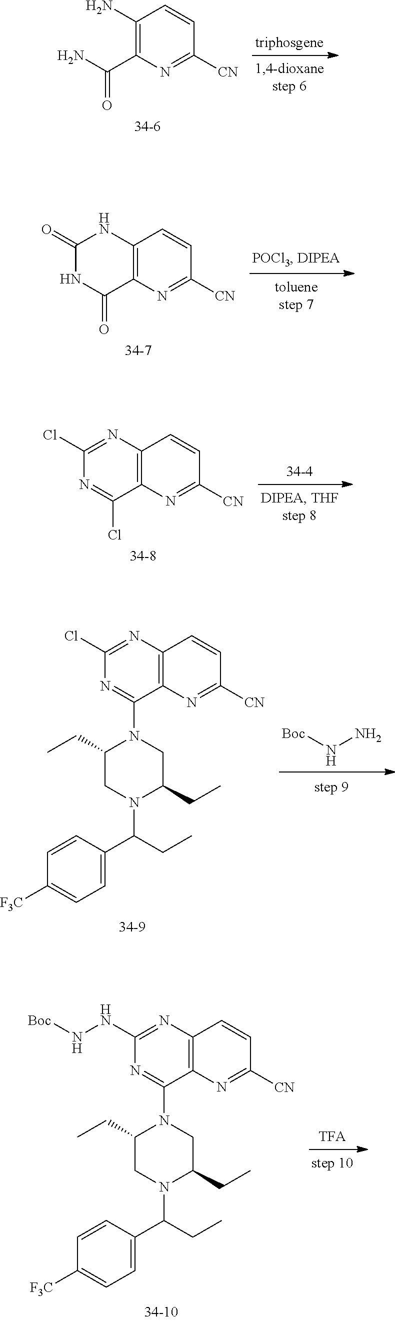 [SMILES: N#Cc1ccc(N)c(C(N)=O)n1 ; N#Cc1ccc2[nH]c(=O)[nH]c(=O)c2n1 ; N#Cc1ccc2nc(Cl)nc(Cl)c2n1 ; CCC(c1ccc(C(F)(F)F)cc1)N1C[C@H](CC)N(c2nc(Cl)nc3ccc(C#N)nc23)C[C@H]1CC ; CC(C)(C)OC(=O)NN ; CCC(c1ccc(C(F)(F)F)cc1)N1C[C@H](CC)N(c2nc(NNC(=O)OC(C)(C)C)nc3ccc(C#N)nc23)C[C@H]1CC]

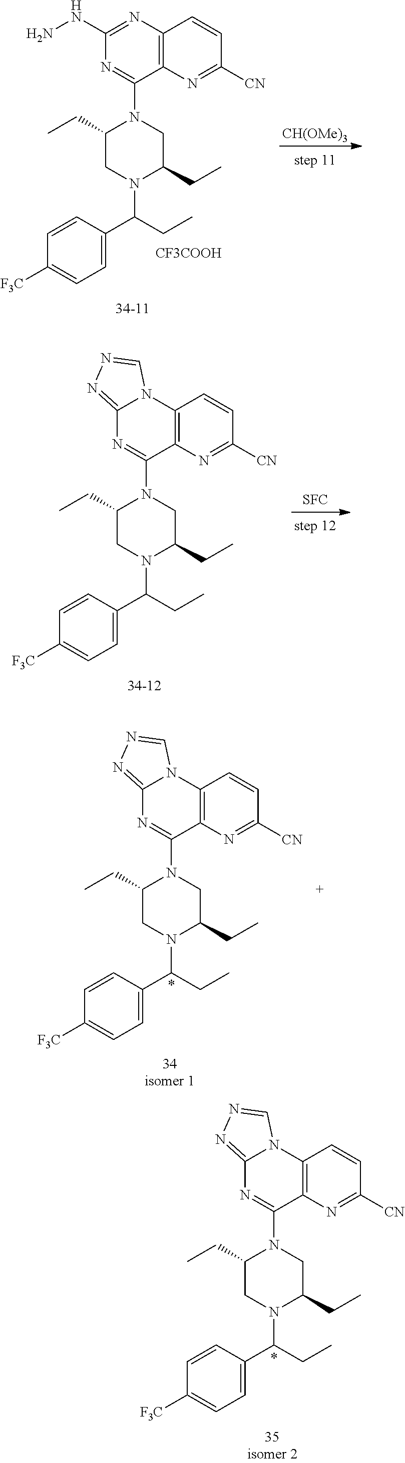

Step 1: Compound 34-2 was prepared from compound 34-1 following the procedure for the synthesis of compound 28-3 in example 11.

Step 2: A mixture of 34-2 (3.2 g, 14.3 mmol), 28-9 (4.0 g, 17.2 mmol), K₂CO₃(5.9 g, 43.0 mmol), NaI (0.2 g, 1.4 mmol) and TBAI (0.5 g, 1.4 mmol) in acetonitrile (80 mL) was stirred at 90° C. for 66 h. The reaction mixture was filtered and concentrated. The residue was purified by column chromatography on silica gel (petroleum ether/ethyl acetate=3/1) to afford 34-3.

Step 3: To a stirred solution of 34-3 (450 mg, 1.1 mmol) in isopropanol (10 mL) was added Pd/C (10%, 45 mg) at 25° C. The mixture was degassed several times with H₂, then the mixture was stirred at 25° C. under H₂ for 21 h. The reaction mixture was filtered and the filtrate was concentrated to afford 34-4.

Step 4: To a mixture of 5-2 (1.0 g, 6.54 mmol) and K₂CO₃ (2.7 g, 19.54 mmol) in DMSO (10 mL) was added H₂O₂(2.2 g, 19.54 mmol) at 0° C., slowly. Then the mixture was stirred at room temperature for 2 h. The reaction mixture was diluted with water and extracted with ethyl acetate. The combined organic layers were washed with water and brine, dried and concentrated under reduced pressure. The residue was purified by column chromatography on silica gel (petroleum ether/ethyl acetate=10/1 to 2/1) to afford 34-5.

Step 5: To a solution of 34-5 (5.0 g, 29.2 mmol) in NMP (100 mL) were added Zn (0.4 g, 5.84 mmol), Zn(CN)₂ (6.9 g, 58.4 mmol), dppf (1.0 g, 1.75 mmol) and Pd₂(dba)₃ (2.7 g, 2.92 mmol) under N₂. The reaction mixture was stirred at 120° C. for 2 h. The reaction mixture was diluted with water and extracted with ethyl acetate. The combined organic layers were washed with water and brine, dried and concentrated under reduced pressure. The residue was purified by column chromatography on silica gel (petroleum ether/ethyl acetate=10/1 to 0/1) to afford 34-6.

Step 6: To a solution of 34-6 (0.5 g, 3.08 mmol) in dioxane (20 mL) was added triphosgene (457.5 mg, 1.54 mmol) under N₂. The reaction mixture was stirred at 100° C. for 3 h. The resulting mixture was cooled to room temperature, filtered and dried to afford 34-7.

Step 7: To a solution of 34-7 (255 mg, 1.36 mmol) in toluene (10 mL) were added POCl₃ (1.26 mL, 13.6 mmol) and DIPEA (0.67 mL, 4.06 mmol). The reaction mixture was stirred at 100° C. for 16 h. The reaction mixture was concentrated to afford 34-8 which was used for the next step directly without further purification.

Step 8: To a solution of 34-8 (300 mg, crude) in THF (10 mL) were added DIPEA (2.2 mL, 13.3 mmol) and 34-4 (437.8 mg, 1.33 mmol). The reaction mixture was stirred at 50° C. for 4 h. The reaction mixture was diluted with water and extracted with ethyl acetate. The combined organic layers were washed with water and brine, dried and concentrated under reduced pressure. The residue was purified by column chromatography on silica gel (petroleum ether/ethyl acetate=10/1 to 0/1) to afford 34-9.

Step 9: To a solution of 34-9 (240 mg, 0.46 mmol) in dioxane (15 mL) were added (tert-butoxy)carbohydrazide (184 mg, 1.39 mmol), Cs₂CO₃ (453.8 mg, 1.39 mmol), tBubrettphos (22.5 mg, 0.046 mmol) and Pd₂(dba)₃ (42.5 mg, 0.046 mmol). The reaction mixture was stirred at 80° C. for 2 h. The reaction mixture was diluted with water and extracted with ethyl acetate. The combined organic layers were washed with water and brine, dried and concentrated under reduced pressure. The residue was purified by column chromatography on silica gel (petroleum ether/ethyl acetate=10/1 to 0/1) to afford 34-10.

Step 10: To a solution of 34-10 (240 mg, 0.39 mmol) in dichloromethane (6 mL) was added TFA (3 mL). The reaction mixture stirred at 25° C. for 1 h. The mixture was concentrated to afford 34-11 which was used in the next step directly without further purification.

Step 11: A mixture of 34-11 (180 mg, crude) and CH(OCH₃)₃(5 mL) was stirred at 80° C. for 1 h. The mixture was concentrated. The residue was purified by column chromatography on silica gel (petroleum ether/ethyl acetate=2/1 to 0/1) to afford 34-12.

Step 12: 34-12 (90 mg) was purified by SFC (column: DAICELCHIRALCEL®AD, EtOH (+0.1% 7.0 mol/1 Ammonia in EtOH)/Supercritical CO₂=40/60) to afford 34 (31.64 mg) and 35 (8.09 mg) respectively. 34: SFC analysis: 100% de; retention time: 4.35 min; column: DAICELCHIRALPAK®AD, EtOH (0.1% DEA) in CO₂, 5% to 40%; pressure: 1800 psi; flow rate: 1.5 mL/min. LCMS (ESI, m/z): [M+H]⁺=523.5. ¹H NMR (400 MHz, methanol-d₄, ppm): δ 9.32 (s, 1H), 8.77-8.75 (m, 1H), 8.31-8.29 (m, 1H), 7.69-7.67 (m, 2H), 7.67-7.55 (m, 2H), 5.96-5.53 (m, 1H), 3.68-3.65 (m, 1H), 3.48-3.39 (m, 2H), 3.12-3.05 (m, 1H), 2.96-2.92 (m, 1H), 2.46 (s, 1H), 2.23 (s, 1H), 2.06-2.00 (m, 2H), 1.73-1.65 (m, 1H), 1.58-1.53 (m, 2H), 1.09-1.06 (m, 3H), 0.78-0.69 (m, 6H). ¹⁹F NMR (376 MHz, methanol-d₄, ppm): δ −63.83 (3F). 35: SFC analysis: 97.18% de; retention time: 4.94 min; column: DAICELCHIRALPAK®AD, EtOH (0.1% DEA) in CO₂, 5% to 40%; pressure: 1800 psi; flow rate: 1.5 mL/min. LCMS (ESI, m/z): [M+H]⁺=523.5. ¹H NMR (400 MHz, methanol-d₄, ppm): δ 9.32 (s, 1H), 8.77-8.75 (m, 1H), 8.30-8.28 (m, 1H), 7.65-7.63 (m, 2H), 7.60-7.58 (m, 2H), 5.32 (s, 1H), 3.66-3.47 (m, 3H), 2.70-2.66 (m, 1H), 2.37-2.33 (m, 1H), 2.20-2.16 (m, 1H), 2.07-1.90 (m, 3H), 1.70-1.56 (m, 3H), 1.13-1.03 (m, 3H), 0.71-0.65 (m, 6H). ¹⁹F NMR (376 MHz, methanol-d₄, ppm): δ −63.82 (3F).

### Example 15 Synthesis of Compound 36 and 37

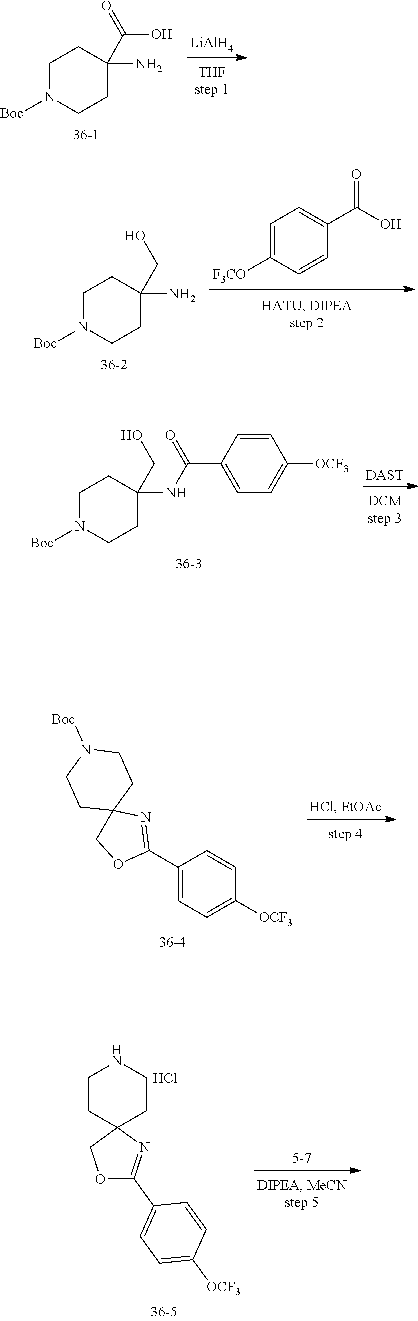

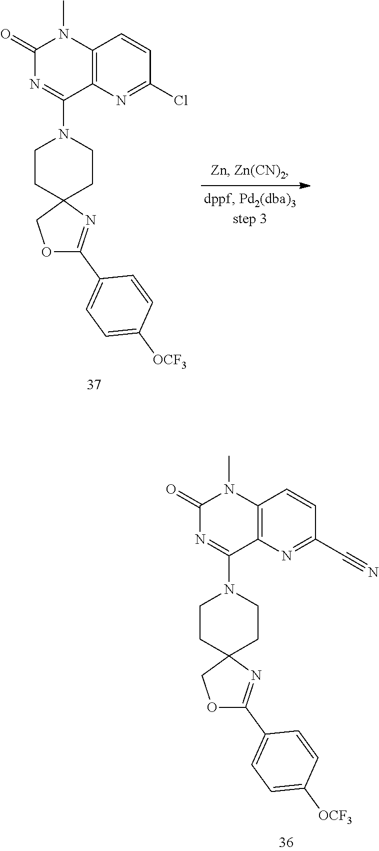 [SMILES: Cn1c(=O)nc(N2CCC3(CC2)COC(c2ccc(OC(F)(F)F)cc2)=N3)c2nc(Cl)ccc21 ; Cn1c(=O)nc(N2CCC3(CC2)COC(c2ccc(OC(F)(F)F)cc2)=N3)c2nc(C#N)ccc21]

Step 1: A mixture of LiAlH₄ (0.30 g, 8.2 mmol) in THF (20 mL) was stirred at 0° C. under N₂ for 0.5 h. Then 36-1 (1 g, 4.1 mmol) was added and the mixture was stirred at 25° C. for 16 h. The mixture was diluted with THF, quenched with saturated Na₂SO₄ (1.5 mL) and filtered. The filtrate was concentrated to afford 36-2 which was used for the next step directly without further purification.

Step 2: A solution of 36-2 (700 mg, crude), 4-(trifluoromethoxy)benzoic acid (626.5 mg, 3.04 mmol), HATU (1733.5 mg, 4.56 mmol) and DIPEA(0.75 mL, 4.56 mmol) in DMF (12 mL) was stirred at 25° C. for 3 h. The mixture was diluted with H₂O and extracted with ethyl acetate. The organic phase was washed with H₂O and concentrated. The residue was purified by column chromatography on silica gel (petroleum ether/ethyl acetate=1/0 to 2/1) to afford 36-3.

Step 3: To a solution of 36-3 (580 mg, 1.39 mmol) in dichloromethane (8 mL) was added DAST (0.28 mL, 2.08 mmol) at 0° C. Then the mixture was stirred at 20° C. for 1 h. The mixture was quenched with NaHCO₃ aqueous, extracted with dichloromethane. The organic phase was concentrated, the residue was purified by column chromatography on silica gel (petroleum ether/ethyl acetate=1/0 to 3/1) to afford 36-4.

Step 4: To a solution of 36-4 (588 mg, 1.47 mmol) in dichloromethane (6 mL) was added HCl in ethyl acetate (4M, 2.5 mL) at 20° C. The mixture was stirred at 20° C. for 1.5 h. The mixture was concentrated under reduced pressure to afford 36-5.

Step 5: A solution of 5-7 (250 mg, 0.87 mmol), 36-5 (208.9 mg, 0.67 mmol) and DIPEA (1.50 mL, 9.1 mmol) in acetonitrile (10 mL) was stirred at 85° C. for 3 h under N₂. The mixture was concentrated. The residue was dissolved in ethyl acetate and washed with H₂O. The organic phase was concentrated. The residue was purified by reverse phase HPLC (acetonitrile/H₂O: 5%-80%) to afford 37. LCMS (ESI, m/z): [M+H]⁺=494.2. ¹H NMR (400 MHz, DMSO-d₆, ppm): δ 8.28-8.23 (m, 1H), 8.09-7.92 (m, 3H), 7.55-7.42 (m, 2H), 4.75-3.90 (m, 6H), 3.46 (s, 3H), 1.98-1.78 (m, 4H). ¹⁹F NMR (376 MHz, DMSO-d₆, ppm): δ −56.68 (3F).

Step 6: A solution of 37 (100 mg, 0.20 mmol), dppf (22.5 mg, 0.040 mmol), Zn (13.2 mg, 0.20 mmol), Zn(CN)₂ (47.6 mg, 0.41 mmol) and Pd₂(dba)₃ (18.5 mg, 0.02 mmol) in NMP (8 mL) was stirred at 90° C. for 16 h under N₂. The mixture was diluted with H₂O and extracted with ethyl acetate. The organic phase was washed with H₂O and concentrated. The residue was purified by reverse phase HPLC (acetonitrile/H₂O: 5%-80%) to afford 36. LCMS (ESI, m/z): [M+H]⁺=485.2. ¹H NMR (400 MHz, DMSO-d₆, ppm): δ 8.13-7.89 (m, 3H), 7.85-7.78 (m, 1H), 7.54-7.43 (m, 2H), 4.80-3.90 (m, 6H), 3.45 (s, 3H), 1.94-1.75 (m, 4H). ¹⁹F NMR (376 MHz, DMSO-d₆, ppm): δ −56.68 (3F).

### Example 16 Synthesis of Compound 39 and 42

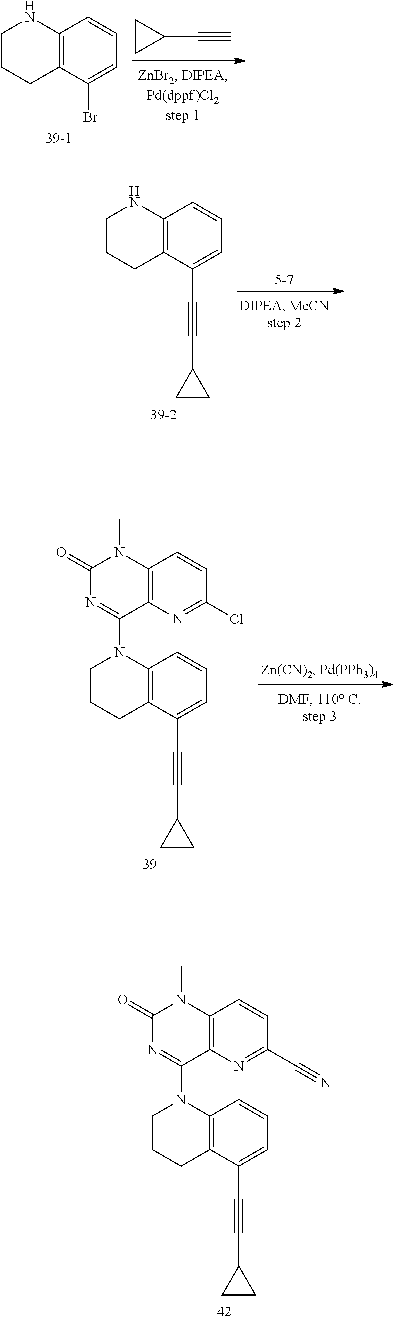

Step 1: To a stirred solution of 39-1 (1 g, 4.71 mmol) in NMP (10 mL) were added ZnBr₂ (10.6 g, 47.15 mmol), Pd(dppf)Cl₂ (0.3 g, 0.47 mmol), DIPEA (15.59 mL, 94.3 mmol) and ethynylcyclopropane (3.12 g, 47.15 mmol) under N₂. The reaction mixture was stirred at 120° C. for 2 h. The reaction mixture was quenched with H₂O and extracted with ethyl acetate. The combined organic layer was washed with brine. The organic layer was dried over Na₂SO₄, filtered and concentrated. The residue was purified by column chromatography on silica gel (petroleum ether/ethyl acetate=1/0 to 20/1) to afford 39-2.

Step 2: To a solution of 5-7 (583 mg, 2.53 mmol) in acetonitrile (5 mL) were added 39-2 (500 mg, 2.53 mmol) and DIPEA (983 mg, 7.6 mmol). The reaction was stirred at room temperature for 3 h under N₂. The reaction mixture was concentrated, the residue was purified by reverse phase HPLC (acetonitrile/H₂O, 5%˜50%) to afford 39. LCMS (ESI, m/z): [M+H]⁺=391.2; ¹H NMR (400 MHz, DMSO-d₆, ppm): δ 7.98 (d, J=8.0 Hz, 1H), 7.75 (d, J=8.0 Hz, 1H), 7.14-7.10 (m, 1H), 6.97-6.90 (m, 2H), 4.06-4.02 (m, 2H), 3.50 (s, 3H), 2.84-2.80 (m, 2H), 1.99-1.93 (m, 2H), 1.63-1.56 (m, 1H), 0.96-0.94 (m, 2H), 0.93-0.91 (m, 2H).

Step 3: To a solution of 39 (140 mg, 0.36 mmol) in DMF (1.5 mL) were added Zn(CN)₂ (420 mg, 3.58 mmol) and Pd(PPh₃)₄(206.8 mg, 0.18 mmol). Then the reaction mixture was stirred at 110° C. for 1 h under N₂. The reaction mixture was concentrated, the residue was purified by reverse phase HPLC (acetonitrile/H₂O: 5%-50%) to afford 42. LCMS (ESI, m/z): [M+H]⁺=382.2; ¹H NMR (400 MHz, DMSO-d₆, ppm): δ 8.19 (d, J=8.0 Hz, 1H), 8.05 (d, J=8.0 Hz, 1H), 7.17-7.13 (m, 1H), 6.96-6.94 (m, 2H), 4.08-4.03 (m, 2H), 3.50 (s, 3H), 2.85-2.82 (m, 2H), 2.01-1.95 (m, 2H), 1.62-1.58 (m, 1H), 0.95-0.93 (m, 2H), 0.92-0.91 (m, 2H).

### Example 17 Synthesis of Compound 44 and 45

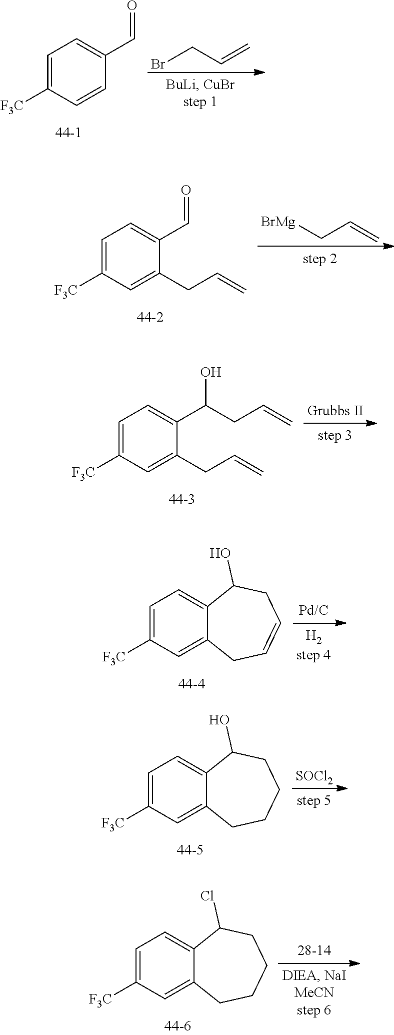

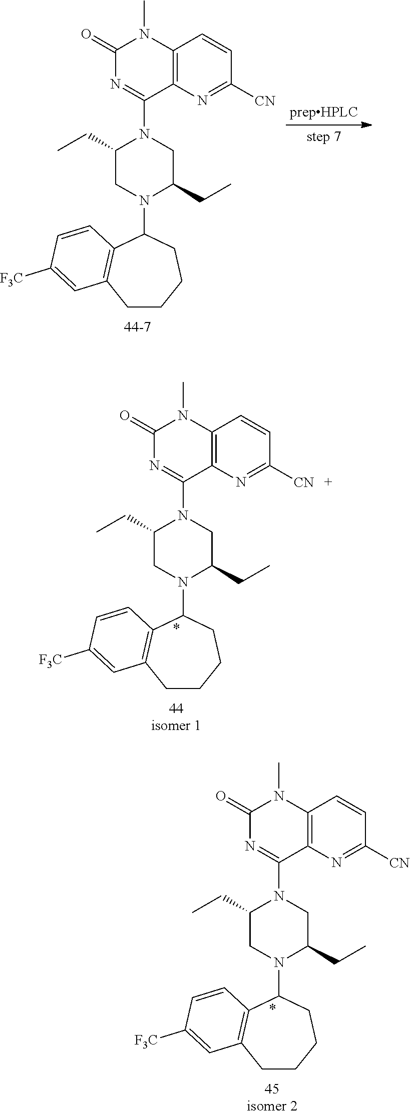

Step 1: To a solution of N₁,N₁,N₂,N₂-tetramethylethane-1,2-diamine (5.8 g, 56.28 mmol) in THF (80 mL) was added n-BuLi (40 mL, 64 mmol, 1.6 mol/L in THF) dropwise at −40° C. under N₂. The reaction was stirred at −40° C. for 40 minutes. Then 44-1 (7 g, 40.2 mmol) was added to the mixture over 5 minutes. The mixture was stirred at −40° C. for 30 minutes. The second batch of n-BuLi (25.328 mL, 40.525 mmol, 1.6 mol/L in THF) was added dropwise over 5 minutes, then the reaction mixture was stirred at −40° C. for 2 h. Remove the cold bath, CuBr (7.5 g, 52.26 mmol) was added to the mixture, and the reaction mixture was stirred at 25° C. for another 1 h. The reaction was cooled to −30° C. and a solution of 3-bromoprop-1-ene (9.73 g, 80.4 mmol) in THF (10 ml) was added dropwise over 5 minutes, then the reaction mixture was stirred at −40° C. for 2 h. The reaction was quenched by addition of methanol. The pH of the mixture was adjusted to 6 with HCl (6M). The reaction mixture was filtered and the filter cake was washed with ethyl acetate. The mixture was extracted with ethyl acetate. The combined organic layer was washed with brine. The combine organic layer was concentrated. The residue was purified by column chromatography on silica gel (petroleum ether/ethyl acetate=1/0 to 3/1) to afford 44-2.

Step 2: To a solution of 44-2 (5 g, 23.34 mmol) in THF (50 mL) was added allylmagnesium bromide (25.68 mL, 25.68 mmol), and the reaction was stirred at −40° C. for 1 h. The reaction was quenched with saturated aqueous NH₄C₁. The reaction mixture was extracted with ethyl acetate. The combined organic layers were washed with brine and concentrated. The residue was purified by column chromatography on silica gel (petroleum ether/ethyl acetate=1/0 to 2/1) to afford 44-3.

Step 3: To a solution of 44-3 (2.0 g, 7.8 mmol) in dichloromethane (160 mL) was added Grubbs 2nd catalyst (331 mg, 0.39 mmol). The reaction was stirred at 20° C. for 18 h under N₂. The reaction mixture was diluted with water. The organic layer was separated, washed with brine and concentrated. The residue was purified by column chromatography on silica gel (petroleum ether/ethyl acetate=1/0 to 2/1) to afford 44-4.

Step 4: To a solution of 44-4 (1.36 g, 5.96 mmol) in EtOH (25 mL) was added 10% Pd/C (0.1 g). The suspension was degassed under vacuum and purged with H₂ 3 times, then the mixture was stirred at 25° C. for 1 h under H₂. The suspension was filtered, and the filter cake was washed with EtOH (20 ml). The combined filtrates were concentrated to afford 44-5.

Step 5: To a solution of 44-5 (1 g, 4.34 mmol) in dichloromethane (25 mL) was added SOC₂ (0.47 mL, 6.51 mmol), and the reaction was stirred at 0° C. for 1.5 h. The reaction was quenched with water. The aqueous layer was extracted with dichloromethane. The combined organic layers were washed with brine. The organic layer was dried over Na₂SO₄, filtered and concentrated. The residue was purified by column chromatography on silica gel (petroleum ether/ethyl acetate=1/0 to 10/1) to afford 44-6.

Step 6: To a solution of 28-14 (250 mg, 0.77 mmol) in acetonitrile (10 mL) were added DIPEA (0.25 mL, 1.53 mmol), NaI (344 mg, 2.3 mmol) and 44-6 (380 mg, 1.53 mmol). The reaction mixture was stirred at 80° C. for 24 h. Then another batch of 44-6 (190 mg, 0.76 mmol) was added to the reaction mixture, and reaction was stirred at 80° C. for another 24 h. The reaction mixture was concentrated in vacuo. The residue was purified by column chromatography on silica gel (dichloromethane/methanol=1/0 to 10/1) to afford 44-7.

Step 7: 44-7 (150 mg, 0.28 mmol) was purified by reverse phase HPLC (acetonitrile/0.1% TFA in water: 75%) to afford 44 (10.43 mg) and 45 (4.01 mg). 44: Analytical HPLC: retention time: 1.85 min; column: Waters ACQUITY BEH C18 2.1*50 mm, 1.7 um; Mobile phaseA: H₂O (0.05% TFA); Mobile phaseB: Acetonitrile (0.05% TFA); flow rate: 1.0 mL/min; Run time: 5 min; 95 to 5% A in 3 min, 5% A for 2 min. LCMS (ESI, m/z): [M+H]⁺=539.4; ¹H NMR (400 MHz, CDCl₃): δ 7.89-7.77 (m, 1H), 7.63-7.54 (m, 1H), 7.40-7.30 (m, 2H), 7.25-7.20 (m, 1H), 6.16-5.54 (m, 1H), 5.34-5.09 (m, 1H), 3.75-3.67 (m, 1H), 3.64-3.34 (m, 5H), 3.12-2.97 (m, 1H), 2.74-2.63 (m, 1H), 2.63-2.52 (m, 1H), 2.18-2.11 (m, 1H), 2.06-1.90 (m, 4H), 1.87-1.75 (m, 2H), 1.73-1.63 (m, 1H), 1.49-1.36 (m, 2H), 1.31-1.25 (m, 1H), 1.09-0.90 (m, 3H), 0.74-0.60 (m, 3H). ¹⁹F NMR (376 MHz, CDCl₃, ppm): δ −62.33 (3F). 45: Analytical HPLC: retention time: 1.85 min; column: Waters ACQUITY BEH C18 2.1*50 mm, 1.7 um; Mobile phaseA: H₂O (0.05% TFA); Mobile phaseB: Acetonitrile (0.05% TFA); flow rate: 1.0 mL/min; Run time: 5 min; 95 to 5% A in 3 min, 5% A for 2 min. LCMS (ESI, m/z): [M+H]⁺=539.4; ¹H NMR (400 MHz, CDCl₃): δ 7.89-7.77 (m, 1H), 7.64-7.54 (m, 1H), 7.46-7.29 (m, 2H), 7.24-7.16 (m, 1H), 6.03-4.80 (m, 2H), 3.77-3.07 (m, 6H), 2.95-2.80 (m, 1H), 2.78-2.53 (m, 2H), 2.30-2.10 (m, 4H), 2.08-1.90 (m, 2H), 1.74-1.67 (m, 2H), 1.41-1.29 (m, 3H), 1.05-0.96 (m, 3H), 0.69-0.37 (m, 3H). ¹⁹F NMR (376 MHz, CDCl₃, ppm): δ −62.34 (3F).

### Example 18 Synthesis of Compound 50

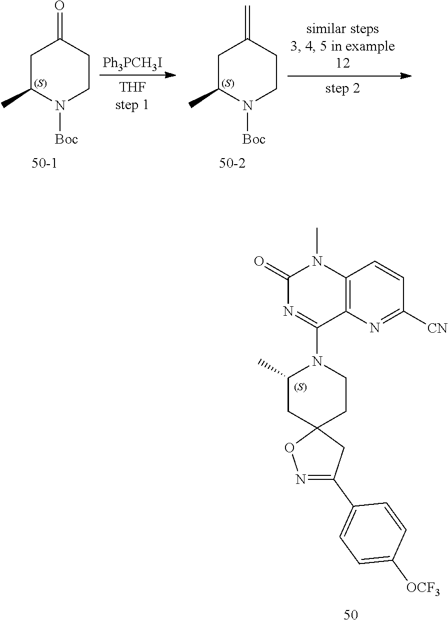

Step 1: To a solution of methyltriphenylphosphonium bromide (4.4 g, 12.19 mmol) in THF (25 mL) was added potassium tert-butoxide (12.19 mL, 12.19 mmol) at 20° C. The mixture was stirred at 55° C. under N₂ for 2 h. Then 50-1 (1.3 g, 6.09 mmol) was added and the mixture was stirred at 55° C. for 16 h. The mixture was concentrated. The residue was purified by column chromatography on silica gel (petroleum ether/ethyl acetate=1/0 to 10/1) to afford 50-2.

Step 2: Compound 50 was prepared from 50-2 following the procedure for the synthesis of compound 30 in example 12. LCMS (ESI, m/z): [M+H]⁺=499.2; ¹H NMR (400 MHz, DMSO-d₆, ppm): δ 8.30-8.23 (m, 1H), 8.06-7.98 (m, 1H), 7.88-7.72 (m, 2H), 7.53-7.42 (m, 2H), 5.60-5.35 (m, 1H), 5.25-4.80 (m, 1H), 3.70-3.60 (m, 1H), 3.46 (s, 3H), 3.42-3.35 (m, 1H), 3.34-3.30 (m, 1H), 2.24-2.10 (m, 1H), 2.07-1.79 (m, 3H), 1.56-1.36 (m, 3H). ¹⁹F NMR (376 MHz, DMSO-d₆, ppm): δ −56.75 (3F).

### Example 19 Synthesis of Compound 57 and 58

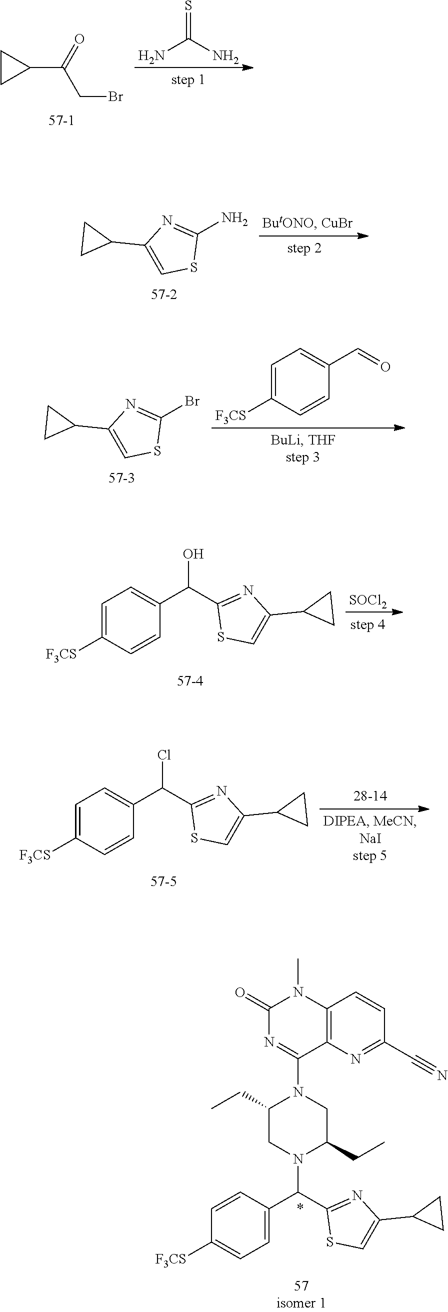

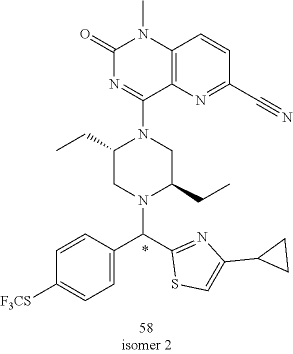

Step 1: To a stirred solution of 57-1 (45 g, 276 mmol) in EtOH (450 mL) was added thiourea (22.06 g, 290 mmol) at room temperature. The resulting mixture was stirred at 80° C. for 15 h under N₂. The solvent was removed under reduced pressure. The residue was diluted with H₂O and neutralized with NaHCO₃ until the pH is 8-9, then dichloromethane was added to the mixture. The mixture was extracted with dichloromethane. The combined organic layers were washed with saturated NaHCO₃ and brine, dried over anhydrous Na₂SO₄ and concentrated to afford 57-2.

Step 2: To a stirred solution of 57-2 (6 g, 19.9 mmol) and CuBr (4.3 g, 29.9 mmol) in acetonitrile (80 mL) was added t-BuONO (3.59 mL, 29.9 mmol) at 0-5° C. The resulting mixture was stirred at room temperature for 30 h. The reaction mixture was filtered and the filtrate was concentrated. The residue was purified by column chromatography on silica gel (petroleum ether/ethyl acetate=1/0 to 6/1) to afford 57-3.

Step 3: To a stirred solution of 57-3 (1.0 g, 4.9 mmol) in THF (20 mL) was added n-BuLi (2.55 mL, 6.37 mmol) below −60° C. under N₂. The resulting mixture was stirred at this temperature for 1 h. A solution of 4-((trifluoromethyl)thio)benzaldehyde (1313.4 mg, 6.37 mmol) in THF (20 mL) was added into the above reaction mixture below −60° C. The resulting mixture was stirred at room temperature for 30 minutes. The reaction mixture was quenched with saturated NH₄Cl and diluted with H₂O. The mixture was extracted with ethyl acetate. The organic layer was combined, washed with brine, dried over anhydrous Na₂SO₄ and concentrated. The residue was purified by column chromatography on silica gel (petroleum ether/ethyl acetate=1/0 to 5/1) to afford 57-4.

Step 4: To a stirred solution of 57-4 (450 mg, 1.36 mmol) in dichloromethane (6 mL) was added SOCl₂ (0.15 mL, 2.04 mmol) at 25° C. The resulting mixture was stirred at 25° C. for 15 h. The reaction mixture was concentrated to afford 57-5 which was used for the next step directly without further purification.

Step 5: To a stirred solution of 57-5 (1.15 g, 2.63 mmol) and 28-14 (0.86 g, 2.63 mmol) in acetonitrile (10 mL) was added DIPEA (1.83 mL, 10.52 mmol) at 25° C. The resulting mixture was stirred at 90° C. for 15 h. The reaction mixture was concentrated. The residue was purified by reverse phase HPLC (acetonitrile/0.05% TFA in H₂O, 0-60%) to afford 57 (11.65 mg) and 58 (6.24 mg). 57: Analytical HPLC: retention time: 1.85 min; column: Waters ACQUITY BEH C18 2.1*50 mm, 1.7 um; Mobile phaseA: H₂O (0.05% TFA); Mobile phaseB: Acetonitrile (0.05% TFA); flow rate: 1.0 mL/min; Run time: 5 min; 95 to 5% A in 3 min, 5% A for 2 min. LCMS (ESI, m/z): [M+H]⁺=640.2; ¹H NMR (400 MHz, methanol-d₄): δ 8.09-8.03 (m, 1H), 8.02-7.95 (m, 1H), 7.75-7.66 (m, 4H), 7.06 (s, 1H), 6.19-5.51 (m, 1H), 5.22-5.12 (m, 1H), 3.68-3.33 (m, 1H), 3.56 (s, 3H), 2.93-2.82 (m, 1H), 2.76-2.64 (m, 2H), 2.46-2.31 (m, 1H), 2.12-1.97 (m, 3H), 1.55-1.40 (m, 2H), 0.98-0.84 (m, 6H), 0.80-0.73 (m, 2H), 0.69-0.65 (m, 1H), 0.58-0.54 (m, 1H). ¹⁹F NMR (376 MHz, methanol-d₄): δ −44.64 (3F). 58: Analytical HPLC: retention time: 1.85 min; column: Waters ACQUITY BEH C18 2.1*50 mm, 1.7 um; Mobile phaseA: H₂O (0.05% TFA); Mobile phaseB: Acetonitrile (0.05% TFA); flow rate: 1.0 mL/min; Run time: 5 min; 95 to 5% A in 3 min, 5% A for 2 min. LCMS (ESI, m/z): [M+H]⁺=640.2; ¹H NMR (400 MHz, CDCl₃): δ 7.86-7.81 (m, 1H), 7.65-7.57 (m, 5H), 6.85 (s, 1H), 6.07-5.62 (m, 1H), 5.68-5.56 (m, 1H), 3.73-3.41 (m, 1H), 3.58 (s, 3H), 2.76-2.55 (m, 2H), 2.52-2.42 (m, 1H), 2.16-1.93 (m, 4H), 1.50-1.39 (m, 2H), 0.93-0.90 (m, 2H), 0.86-0.81 (m, 6H), 0.78-0.67 (m, 2H). ¹⁹F NMR (376 MHz, CDCl₃): δ −42.58 (3F).

### Example 20 Synthesis of Compound 59

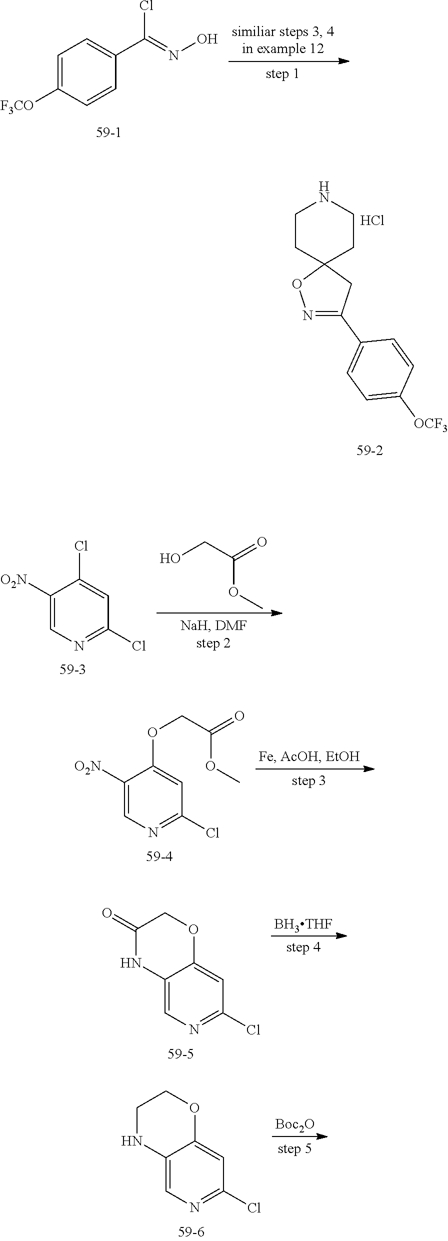

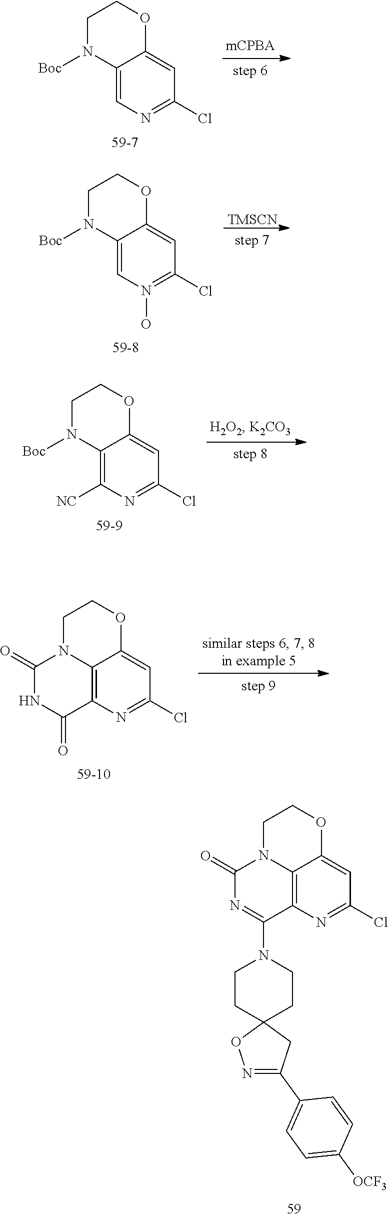

Step 1: Compound 59-2 was prepared from 59-1 following the procedure for the synthesis of compound 30-5 in example 12.

Step 2: To a mixture of 59-3 (4.9 g, 25.4 mmol) and methyl 2-hydroxyacetate (2.74 g, 30.4 mmol) in DMF (50 mL) was added NaH (1.22 g, 60%, 30.5 mmol) at 0° C. Then the mixture was stirred at room temperature for 1 h. H₂O was added to the reaction mixture and the mixture was extracted with ethyl acetate. The combined organic layers were dried over Na₂SO₄, filtered and the filtrate was concentrated. The residue was purified by column chromatography on silica gel (petroleum ether/ethyl acetate=1/0 to 2/1) to afford 59-4.

Step 3: To a solution of 59-4 (4.9 g, 19.87 mmol) in EtOH (50 mL) and HOAc (20 mL) was added Fe (5.6 g, 100 mmol) at room temperature. Then the mixture was stirred at 80° C. for 2 h. The mixture was filtered and the filtrate was concentrated. The residue was purified by column chromatography on silica gel (dichloromethane/methanol=1/0 to 20/1) to afford 59-5.

Step 4: To a solution of 59-5 (2.6 g, 14.09 mmol) in THF (20 mL) was added BH₃-THF (1M, 65 mL, 65 mmol) at room temperature. Then the mixture was stirred at 80° C. for 2 h. The mixture was quenched with methanol and stirred at 80° C. for 30 minutes. Then the mixture was concentrated to afford 59-6 which was used for the next step directly without further purification.

Step 5: To a solution of 59-6 (1.0 g, crude) and (Boc)₂O (4.04 mL, 17.59 mmol) in THF (15 mL) was added DMAP (1.43 g, 11.72 mmol) and triethylamine (0.82 mL, 5.86 mmol) at room temperature. Then the mixture was stirred at 80° C. for 12 h. The mixture was concentrated, the residue was purified by column chromatography on silica gel (dichloromethane/methanol=1/0 to 20/1) to afford 59-7.

Step 6: To a solution of 59-7 (1.5 g, 5.54 mmol) in CHCl₃ (20 mL) was added mCPBA (2.25 g, 11.08 mmol) at room temperature. Then the mixture was stirred at 80° C. for 12 h. This mixture was concentrated and the residue was purified by column chromatography on silica gel (dichloromethane/methanol=1/0 to 10/1) to afford 59-8.

Step 7: To a solution of 59-8 (1.2 g, 4.19 mmol) in acetonitrile (20 mL) were added TMSCN (1.25 g, 12.56 mmol) and triethylamine (1.75 mL, 12.56 mmol) at room temperature. Then the mixture was stirred at 80° C. for 12 h. The mixture was concentrated and the residue was purified by column chromatography on silica gel (petroleum ether/ethyl acetate=1/0 to 4/1) to afford 59-9.

Step 8: To a solution of 59-9 (620 mg, 2.1 mmol) and K₂CO₃ (870 mg, 6.29 mmol) in DMSO (10 mL) was added H₂O₂(30%, 713.0 mg, 6.29 mmol) at 0° C. Then the mixture was stirred at room temperature for 1 h. The solid was collected by filtration and washed with dichloromethane. The solid was suspended in H₂O, then conc. HCl was added until the pH was 4-5. The solid was collected by filtration to afford 59-10.

Step 9: Compound 59 was prepared from 59-10 following the procedure for the synthesis of compound 5 in example 5. LCMS (ESI, m/z): [M+H]⁺=513.2; ¹H NMR (400 MHz, DMSO-d6, ppm): δ 7.89 (s, 1H), 7.84-7.76 (m, 2H), 7.53-7.44 (m, 2H), 5.06-4.58 (m, 1H), 4.55-4.46 (m, 2H), 4.48-4.06 (m, 2H), 4.05-3.68 (m, 3H), 3.35 (s, 2H), 2.06-1.82 (m, 4H). ¹⁹F NMR (376 MHz, DMSO-d6, ppm): δ −56.75 (3F).

### Example 21 Synthesis of Compound 64

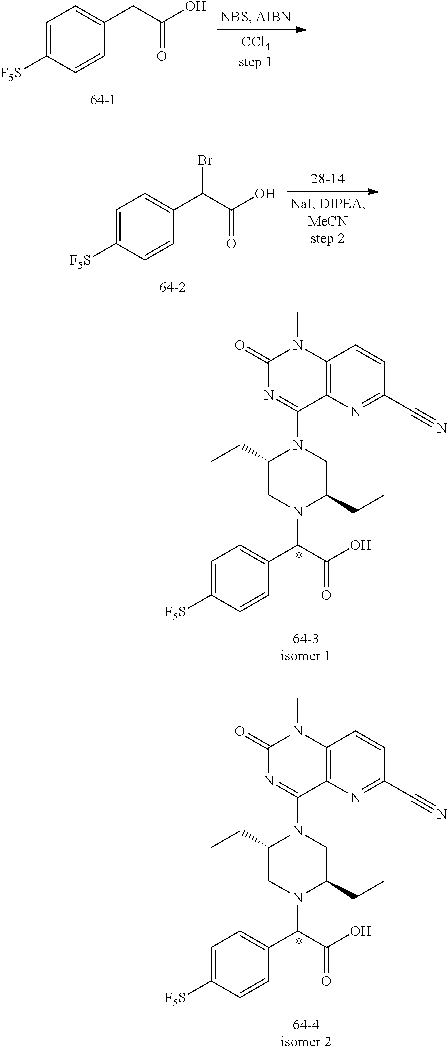

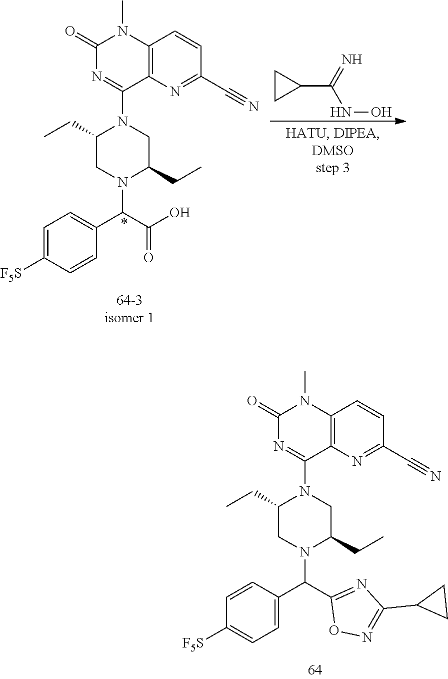

Step 1: To a suspension of 64-1 (950 mg, 3.62 mmol) in CCl₄ (20 mL) was added NBS (709.4 mg, 3.99 mmol) and AIBN (89.2 mg, 0.54 mmol). Then the mixture was stirred at 85° C. for 3 h. The mixture was concentrated and the residue was purified by column chromatography on silica gel (dichloromethane/methanol=1/0 to 10/1) to afford 64-2.

Step 2: To a mixture of 64-2 (500 mg, 1.17 mmol) and 28-14 (421.05 mg, 1.29 mmol) in acetonitrile (15 mL) was added DIPEA (0.97 mL, 5.86 mmol) and NaI (351.6 mg, 2.35 mmol). Then the mixture was stirred at 85° C. for 3 h. The mixture was extracted with ethyl acetate. The organic layer was washed with H₂O and brine (20 mL), dried over Na₂SO₄, filtered and concentrated. The residue was purified by reverse phase HPLC (acetonitrile/0.05% TFA in water: 0-60%) to afford 64-3 and 64-4.

Step 3: To a mixture of 64-3 (75 mg, 0.13 mmol) and N-[azanylidene (cyclopropyl)methyl]hydroxylamine (14.08 mg, 0.14 mmol) in DMSO (2 mL) was added DIPEA (58.3 mg, 0.15 mmol) and HATU (58.3 mg, 0.15 mmol). The mixture was stirred at room temperature for 30 minutes, then the mixture was heated to 90° C. and stirred for 30 minutes. The mixture was cooled and extracted with ethyl acetate. The organic layer was washed with H₂O and brine, dried over anhydrous Na₂SO₄ and concentrated. The residue was purified by reverse phase HPLC (acetonitrile/0.05% TFA in water: 0-70%) to afford 64 as 3.3 eq of TFA salt. LCMS (ESI, m/z): [M+H]⁺=651.4; ¹H NMR (400 MHz, DMSO-d₆, ppm): δ 8.24 (d, J=8.8 Hz, 1H), 8.02-7.98 (m, 3H), 7.88-7.81 (m, 2H), 6.00-5.37 (m, 2H), 4.98-4.91 (m, 1H), 3.65-3.56 (m, 0.5H), 3.44 (s, 3H), 3.30-3.18 (m, 1H), 2.90-2.82 (m, 0.5H), 2.2-2.57 (m, 1H), 2.40-2.21 (m, 2H), 2.17-2.12 (m, 1H), 2.04-1.78 (m, 2H), 1.63-1.39 (m, 2H), 1.11-1.04 (m, 2H), 0.90-0.69 (m, 7H). ¹⁹F NMR (376 MHz, DMSO-d₆, ppm): δ 87.41-86.60 (1F), 64.20-63.80 (4F), −73.59 (9.9F, TFA).

### Example 22 Synthesis of Compound 67

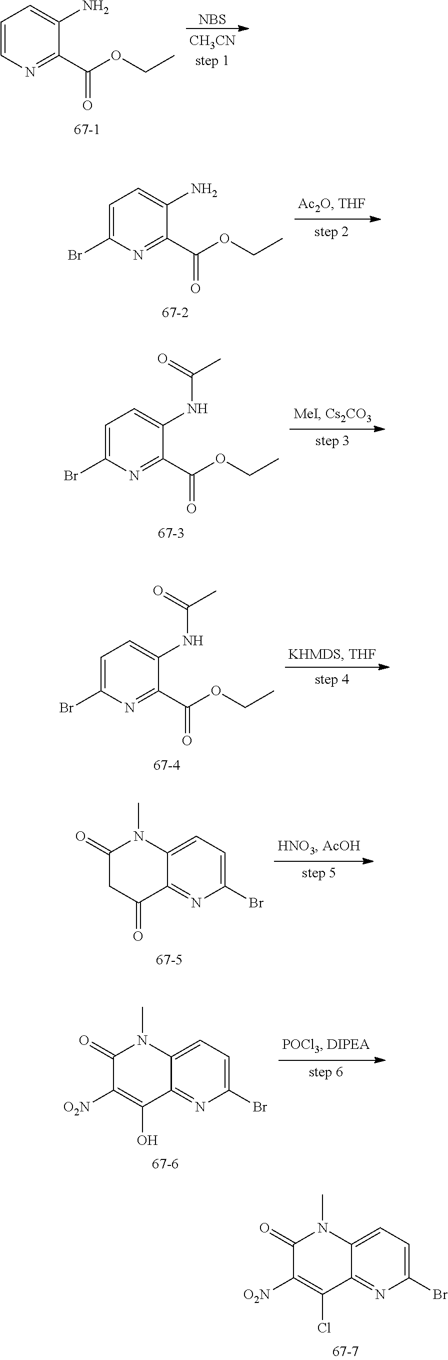

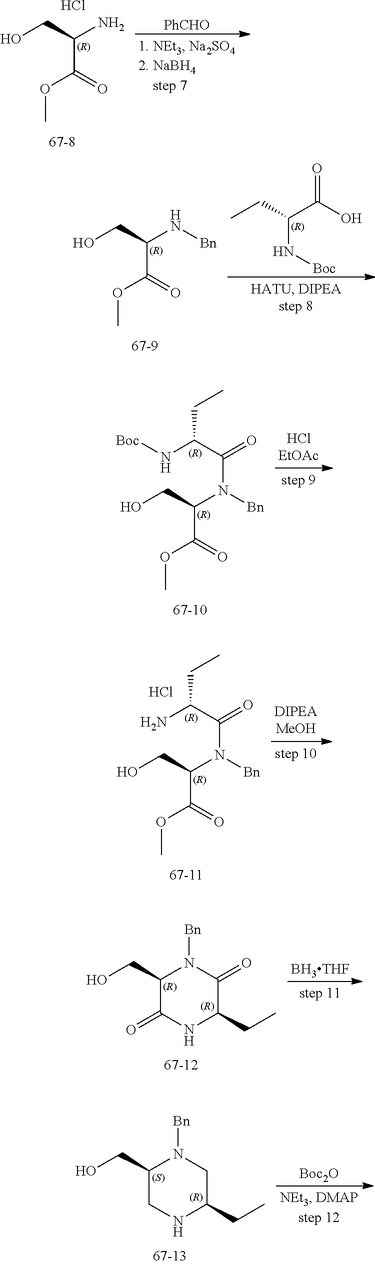

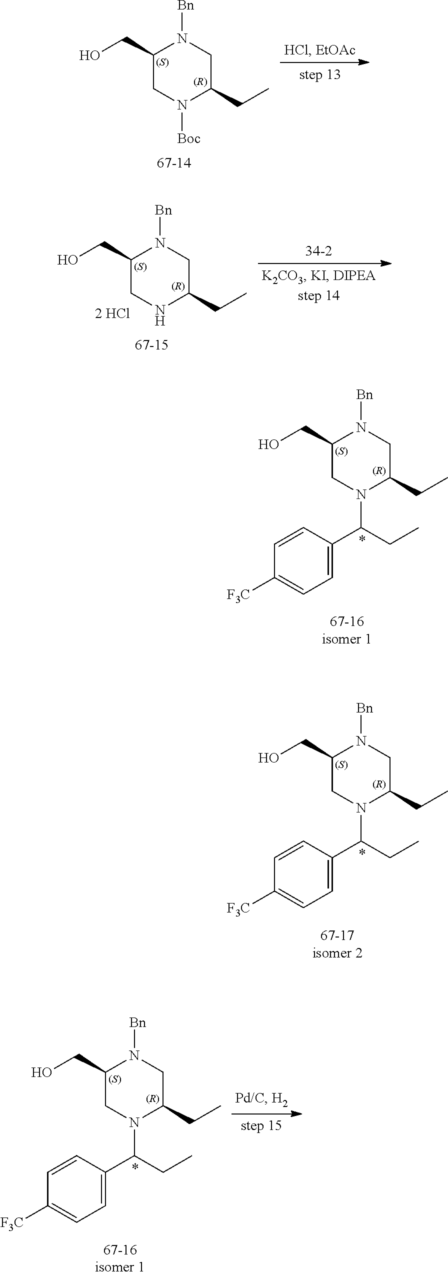

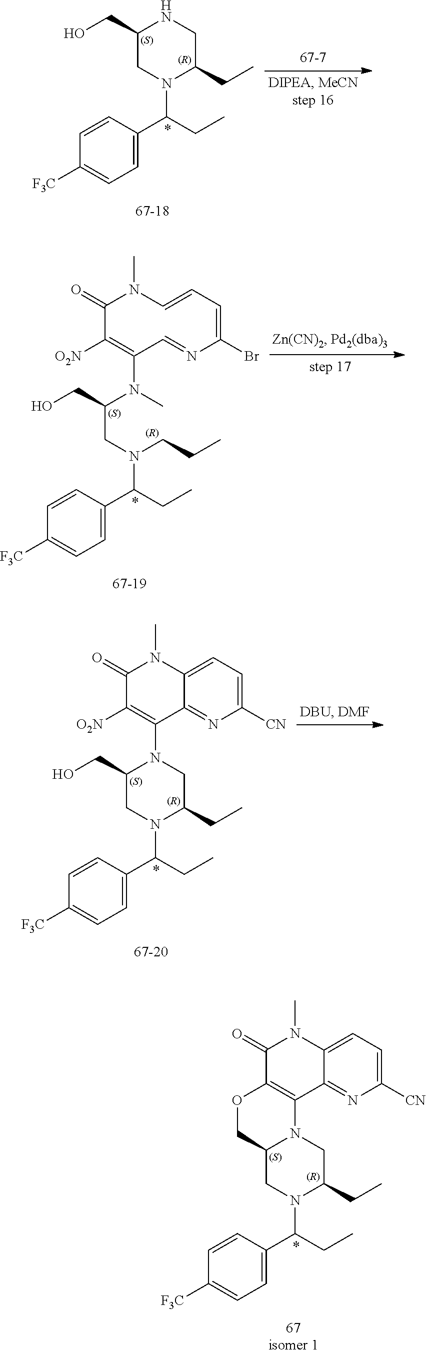

Step 1: To a stirred solution of 67-1 (25 g, 150.4 mmol) in acetonitrile (250 mL) was added NBS (28.1 g, 158 mmol) in portions at 25-40° C. The reaction mixture was stirred at 25-40° C. for 2 h. The precipitate was filtered and the cake was washed with acetonitrile. The cake was collected and dried to afford 67-2.

Step 2: To a stirred solution of 67-2 (20 g, 81.6 mmol) in THF (100 mL) was added dropwise acetyl acetate (24.14 mL, 257.06 mmol) at 25° C. The reaction mixture was stirred at 75° C. for 19 h. The reaction mixture was concentrated. The residue was diluted with H₂O and extracted with ethyl acetate. The organic layer was combined, dried over anhydrous Na₂SO₄ and concentrated to afford 67-3.

Step 3: To a solution of 67-3 (20.9 g, 72.8 mmol) and Cs₂CO₃ (52.2 g, 160.15 mmol) in DMF (150 mL) was added dropwise CH₃I (7.7 mL, 123.7 mmol) at 5-15° C. The reaction mixture was stirred at 25° C. for 67 h. The reaction mixture was diluted with H₂O and extracted with ethyl acetate. The organic layer was combined, washed with H₂O and brine, dried over anhydrous Na₂SO₄ and concentrated. The residue was purified by column chromatography on silica gel (petroleum ether/ethyl acetate=1/0 to 1/4) to afford 67-4.

Step 4: KHMDS in THF (1M, 54.6 mL, 54.6 mmol) was added to THF (200 mL) at −10° C. under N₂, then 67-4 (13.7 g, 45.5 mmol) in THF (400 mL) was added to the solution at −60° C. to −50° C. under N₂. Then the reaction mixture was stirred at 20° C. for 1.5 h. The reaction was quenched with H₂O, and the mixture was extracted with ethyl acetate. The aqueous layer was acidified with HCl (2 N) to change the pH to 3-4. The precipitate was filtered, washed with H₂O and dried to afford 67-5.

Step 5: To a stirred solution of 67-5 (2 g, 7.84 mmol) in AcOH (20 mL) was added HNO₃ (1.3 mL, 31.74 mmol) at 25° C. The reaction mixture was stirred at 80° C. for 15 h. The reaction mixture was cooled to 25° C. and diluted with H₂O. The mixture was stirred for 10 minutes and the precipitation was filtered and dried to afford 67-6.

Step 6: To a stirred solution of 67-6 (1.4 g, 4.67 mmol) in toluene (28 mL) was added DIPEA (4.06 mL, 23.33 mmol) and POCl₃ (2.17 mL, 23.33 mmol) at 25° C. The reaction mixture was stirred at 110° C. for 3 h under N₂. The reaction mixture was concentrated to afford 67-7 which was used for the next step directly without further purification.

Step 7: To a stirred solution of 67-8 (34.9 g, 292.99 mmol) in methanol (250 mL) was added triethylamine (42.76 mL, 307.63 mmol) and benzaldehyde (31.27 mL, 307.63 mmol) at 5-10° C. The reaction mixture was stirred at this temperature for 3 h. Then the reaction mixture was cooled to 0° C. and NaBH₄ (22.2 g, 585.964 mmol) was added into the reaction mixture in portions. The reaction mixture was stirred at 0-20° C. for 2.5 h. The reaction mixture was quenched with HCl (3 N) and extracted with ethyl acetate. The organic layer was washed with HCl (3 N). The aqueous layer was combined and neutralized with NaHCO₃ powder. The aqueous layer was concentrated. The residue was dissolved in dichloromethane/methanol (10:1) and filtered. The organic layer was concentrated, the residue was purified by column chromatography on silica gel (dichloromethane/methanol=1/0 to 10/1) to afford 67-9.

Step 8: To a solution of 67-9 (32.86 g, 157.04 mmol) and (2R)-2-{[(tert-butoxy)carbonyl]amino}butanoic acid (31.92 g, 157.04 mmol) in DMF (350 mL) was added dropwise DIPEA (82.06 mL, 471.11 mmol) at 25° C., then HATU (89.6 g, 235.56 mmol) was added to the solution at 5-15° C. The reaction mixture was stirred at 25° C. for 48 h. The reaction mixture was diluted with H₂O and extracted with ethyl acetate. The organic layer was combined, washed with H₂O and brine, dried over anhydrous Na₂SO₄ and concentrated. The residue was purified by column chromatography on silica gel (petroleum ether/ethyl acetate=1/0 to 0/1) to afford 67-10.

Step 9: To a stirred solution of 67-10 (28.9 g, 73.26 mmol) in methanol (280 mL) was added HCl in ethyl acetate (4M, 50 mL) at 25-35° C. The reaction mixture was stirred at room temperature for 16 h. The reaction mixture was concentrated to afford 100-11.

Step 10: A solution of 67-11 (25.4 g, 69.14 mmol) in DIPEA (35 mL, 200.93 mmol) and methanol (300 mL) was stirred at 70° C. for 15 h. The reaction mixture was concentrated. The residue was purified by column chromatography on silica gel (dichloromethane/methanol=1/0 to 10/1) to afford 67-12.

Step 11: To a stirred solution of 67-12 (21 g, 80.06 mmol) in THF (100 mL) was added BH₃·THF (1M in THF, 22.9 mL, 22.9 mmol) at 0° C. under N₂. The reaction mixture was stirred at 70° C. for 18 h. The reaction mixture was cooled to 0-10° C., then quenched with methanol and HCl (2 N). The resulting mixture was stirred at 70° C. for 2 h. The reaction mixture was concentrated. The residue was diluted with H₂O (100 mL) and basified with NaHCO₃ to pH=8-9. The mixture was concentrated to dryness. The residue was dissolved in a mixture of 10% methanol in dichloromethane and the suspension was filtered. The filtrate was concentrated to afford 67-13 which was used for the next step directly without further purification.

Step 12: To a stirred solution of 67-13 (27.7 g, crude), DMAP (0.9 g, 7.09 mmol) and triethylamine (19.72 mL, 141.85 mmol) in dichloromethane (280 mL) was added (Boc)₂O (14.66 mL, 63.83 mmol) at 0-10° C. The reaction mixture was stirred at room temperature for 15 h. The reaction mixture was concentrated. The residue was purified by column chromatography on silica gel (petroleum ether/ethyl acetate=1/0 to 2/1) to afford 67-14.

Step 13: To a stirred solution of 67-14 (5 g, 14.95 mmol) in methanol (30 mL) was added HCl in ethyl acetate (4M, 30 mL) at 25° C., the reaction mixture was stirred at 25° C. for 15 h. The reaction mixture was concentrated to afford 67-15 which was used for the next step directly without further purification.

Step 14: A mixture of 67-15 (4.5 g, 14.61 mmol), 34-2 (8.13 g, 36.53 mmol), DIPEA (10.18 mL, 58.44 mmol), K₂CO₃ (8.1 g, 58.44 mmol) and KI (4.9 g, 29.22 mmol) in CH₃CN (100 mL) was stirred at 90° C. for 48 h. The reaction mixture was concentrated. The residue was purified by column chromatography on silica gel (petroleum ether/ethyl acetate=1/0 to 2/3) to afford 67-16 and 67-17.

Step 15: To a stirred solution of 67-16 (1.93 g, 4.59 mmol) in isopropanol (60 mL) was added Pd/C (5%, 450 mg) at 25° C. The reaction mixture was degassed with H₂ several times, then the mixture was stirred at 60° C. for 15 h under H₂. The reaction mixture was filtered and the filtrate was concentrated to afford 67-18.

Step 16: A solution of 67-18 (1.6 g, 4.84 mmol), 67-7 (1.46 g, crude) and DIPEA (8.44 mL, 48.43 mmol) in CH₃CN (30 mL) was stirred at 90° C. for 15 h. The reaction mixture was concentrated. The residue was purified by column chromatography on silica gel (petroleum ether/ethyl acetate=1/0 to 1/4) to afford 67-19.

Step 17: To a stirred solution of 67-19 (700 mg, 1.14 mmol), Zn(CN)₂ (268.5 mg, 2.29 mmol), Zn (15.0 mg, 0.23 mmol) and dppf (38.0 mg, 0.069 mmol) in NMP (7 mL) was added Pd₂(dba)₃ (104.7 mg, 0.114 mmol) at 25° C. The reaction mixture was degassed through N₂ for a while and stirred at 90° C. for 15 h. The reaction mixture was diluted with H₂O and extracted with ethyl acetate. The organic layer was combined, washed with H₂O and brine, dried over anhydrous Na₂SO₄ and concentrated. The residue was purified by column chromatography on silica gel (petroleum ether/ethyl acetate=1/0 to 1/4) to afford 67-20.

Step 18: To a stirred solution of 67-20 (180 mg, 0.31 mmol) in DMF (1.8 mL) was added DBU (139.8 mg, 0.92 mmol) at 25° C. The reaction mixture was stirred at 130° C. for 15 h. The reaction mixture was diluted with H₂O and extracted with ethyl acetate. The organic layer was combined, washed with brine, dried over anhydrous Na₂SO₄ and concentrated. The residue was purified by column chromatography on silica gel (petroleum ether/ethyl acetate=1/0 to 0/1) and then by reverse phase HPLC (MeCN/0.1% TFA in water: 0-60%) to afford 67. Analytical HPLC: retention time: 1.69 min; column: Waters ACQUITY BEH C18 2.1*50 mm, 1.7 um; Mobile phaseA: H₂O (0.05% TFA); Mobile phaseB: Acetonitrile (0.05% TFA); flow rate: 1.0 mL/min; Run time: 5 min; 95 to 5% A in 3 min, 5% A for 2 min. LCMS (ESI, m/z): [M+H]⁺=512.2; ¹H NMR (400 MHz, methanol-d₄, ppm): δ 8.06-7.99 (m, 1H), 7.89-7.85 (m, 1H), 7.68-7.62 (m, 2H), 7.59-7.53 (m, 2H), 5.13-5.08 (m, 1H), 4.41-4.35 (m, 1H), 4.08-4.04 (m, 1H), 3.82-3.74 (m, 1H), 3.71 (s, 3H), 3.63-3.52 (m, 1H), 3.16-3.09 (m, 1H), 3.09-3.02 (m, 1H), 2.74-2.65 (m, 1H), 2.52-2.43 (m, 1H), 2.12-2.01 (m, 1H), 1.81-1.59 (m, 3H), 0.81 (t, J=7.4 Hz, 3H), 0.69 (t, J=7.3 Hz, 3H). ¹⁹F NMR (376 MHz, methanol-d₄, ppm): δ −63.86 (3F).

### Example 23 Synthesis of Compound 72 and 73

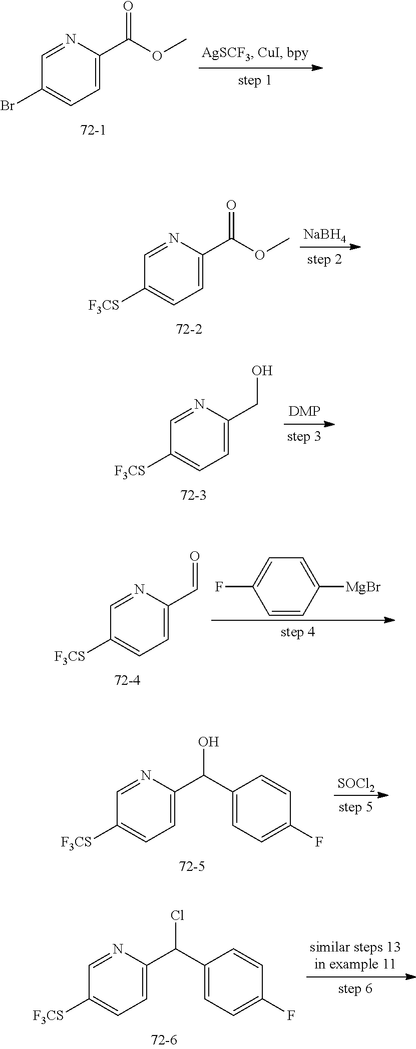

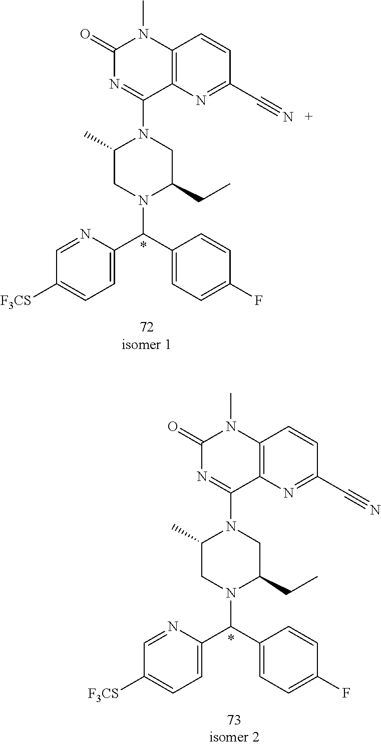

Step 1: To a mixture of methyl 72-1 (500 mg, 2.31 mmol) in 2-methoxyethyl ether (10 mL) was added 2,2′-bipyridine (542 mg, 3.47 mmol), AgSCF₃ (725.4 mg, 3.47 mmol) and CuI (661 mg, 3.47 mmol). Then the mixture was stirred at 130° C. for 24 h under N₂. The mixture was quenched by addition of water, the aqueous layer was extracted with ethyl acetate. The organic extracts were combined, washed with brine, dried over anhydrous Na₂SO₄, the mixture was concentrated. The residue was purified by reverse phase HPLC (MeCN/H₂O (0.05% TFA), 5-95%) to afford 72-2.

Step 2: To a solution of 72-2 (2.2 g, 9.28 mmol) in MeOH (2 mL) was added NaBH₄ (0.7 g, 18.55 mmol) at 0° C. and the reaction mixture was stirred at room temperature for 2 h. The reaction mixture was diluted with DCM and saturated NH₄Cl solution. The organic layer was separated and concentrated in vacuo. The residue was purified by column chromatography on silica gel (petroleum ether/ethyl acetate=10/1) to afford 72-3.

Step 3: To a solution of 72-3 (1.12 g, 5.35 mmol) in DCM (15 mL) was added Dess-Martin periodinane (3.0 g, 6.96 mmol) at 0° C. and the reaction mixture was stirred at room temperature for 30 minutes. The reaction mixture was concentrated in vacuo. The residue was purified by column chromatography on silica gel (petroleum ether/ethyl acetate=10/1) to afford 72-4.

Step 4: To a solution of 72-4 (460 mg, 2.21 mmol) in THF (10 mL) was added 4-fluorophenylmagnesium bromide (0.32 mL, 0.32 mmol) at −78° C., then the reaction mixture was stirred at room temperature for 30 minutes. The reaction mixture was diluted with ethyl acetate and saturated NH₄Cl solution. The organic layer was separated and washed with saturated NaCl solution, then organic layer was concentrated in vacuo. The residue was purified by column chromatography on silica gel (petroleum ether/ethyl acetate=4/1) to afford 72-5.

Step 5: To a solution of 72-5 (580 mg, 1.91 mmol) in DCM (12 mL) were added DMF (0.01 mL, 0.13 mmol) and SOCl₂ (378 mg, 3.82 mmol) at 0° C., then the reaction mixture was stirred at room temperature for 30 minutes. The reaction mixture was concentrated in vacuo to afford 72-6 which was used for the next step directly without further purification.

Step 6: Compound 72 and 73 was prepared from compound 72-6 following the procedure for the synthesis of compound 28-15 in example 11. The product was obtained by reverse phase HPLC (MeCN/water (0.05% TFA), 0-80%). 72: 2.23 TFA salts, LCMS (ESI, m/z): [M+H]⁺=598.4; ¹H NMR (400 MHz, Methanol-d₄, ppm): δ 8.98 (s, 1H), 8.22 (dd, J=8.0, 2.0 Hz, 1H), 8.14-8.01 (m, 2H), 7.76-7.60 (m, 3H), 7.25 (t, J=8.4 Hz, 2H), 6.43-5.91 (m, 2H), 5.42-5.32 (m, 1H), 4.10-3.99 (m, 1H), 3.60 (s, 3H), 3.20-3.02 (m, 2H), 1.91-1.59 (m, 5H), 1.39-1.28 (m, 1H), 0.85 (t, J=7.2 Hz, 3H). ¹⁹F NMR (376 MHz, Methanol-d₄, ppm): δ −44.15 (3F), −77.27(6.7F, TFA), −111.5(1F). 73: 1.48 TFA salts, LCMS (ESI, m/z): [M+H]⁺=598.4; ¹H NMR (400 MHz, Methanol-d₄, ppm): δ 8.93 (s, 1H), 8.21 (dd, J=8.0, 2.0 Hz, 1H), 8.10 (d, J=8.8 Hz, 1H), 8.03 (d, J=8.8 Hz, 1H), 7.81-7.67 (m, 3H), 7.18 (t, J=8.4 Hz, 2H), 6.34-5.96 (m, 1H), 5.55-5.23 (m, 2H), 4.10-3.85 (m, 1H), 3.59 (s, 3H), 3.28-3.15 (m, 1H), 3.10-2.90 (m, 1H), 1.65-1.28 (m, 6H), 0.78-0.64 (m, 3H). ¹⁹F NMR (376 MHz, Methanol-d₄, ppm): δ −4.24 (3F), −77.38 (4.45F, TFA), −111.5 (1F).

### Example 24 Synthesis of Compound 77 and 78

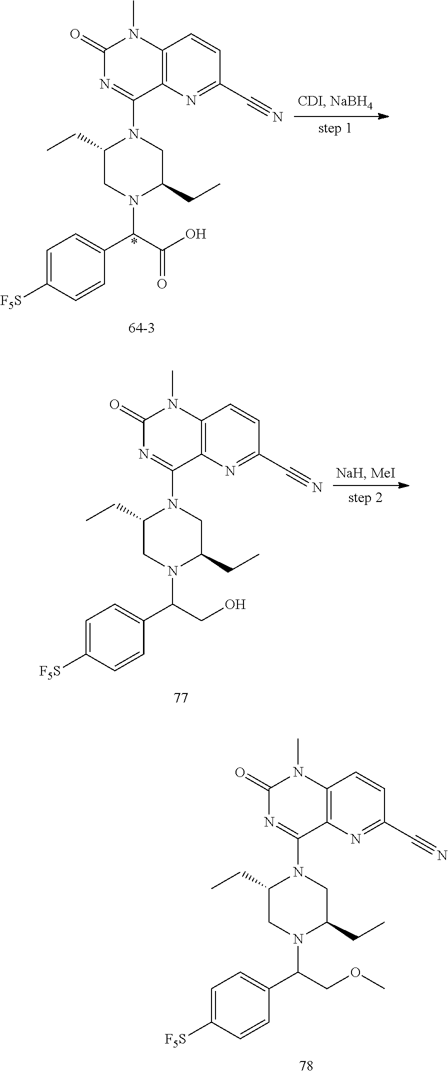

Step 1: To a mixture of 64-3 (80 mg, 0.14 mmol) in THF (10 mL) was added CDI (44.2 mg, 0.27 mmol). After stirred for 16 h at room temperature, NaBH₄ (5.2 mg, 0.14 mmol) was added to the mixture. Then the mixture was stirred at room temperature for 20 minutes. The mixture was quenched by addition of water, the aqueous layer was extracted with ethyl acetate. The organic layer was combined and washed with brine, dried over anhydrous Na₂SO₄. The solvent was concentrated. The residue was purified by column chromatography on silica gel (DCM/MeOH=10/1) to afford 77. LCMS (ESI, m/z): [M+H]⁺=573.3; ¹H NMR (400 MHz, DMSO-d₆, ppm): δ 8.30-8.18 (m, 1H), 8.01-7.91 (m, 1H), 7.90-7.78 (m, 2H), 7.65-7.06 (m, 2H), 6.12-5.57 (m, 0.5H), 5.62-5.53 (m, 0.5H), 5.11-4.71 (m, 2H), 3.81-3.64 (m, 5H), 3.06-2.72 (m, 3H), 2.61-2.53 (m, 1H), 2.37-2.11 (m, 1H), 2.09-1.47 (m, 2H), 1.2-1.41 (m, 2H), 1.07-0.84 (m, 3H), 0.79-0.56 (m, 3H). ¹⁹F NMR (376 MHz, DMSO-d₆, ppm): δ 88.41 (1F), 64.39 (4F).

Step 2: To a mixture of 77 (50 mg, 0.087 mmol) in THF (2 mL) was added NaH (7.0 mg, 0.17 mmol) and iodomethane (37.0 mg, 0.26 mmol). Then the mixture was stirred at 0° C. for 3 h. The reaction mixture was quenched by water, the aqueous layer was extracted with ethyl acetate. The organic layer was combined and washed with brine, dried over anhydrous Na₂SO₄. The solvent was concentrated. The residue was purified by reverse phase HPLC (MeCN/water from 1/0 to 2/3) to afford 78. LCMS (ESI, m/z): [M+H]⁺=587.3; ¹H NMR (400 MHz, DMSO-d6, ppm): δ 8.31-8.17 (m, 1H), 8.03-7.94 (m, 1H), 7.96-7.83 (m, 2H), 7.70-7.58 (m, 2H), 6.09-5.78 (m, 0.5H), 5.53-5.26 (m, 0.5H), 5.09-4.75 (m, 1H), 3.98-3.80 (m, 1H), 3.69-3.48 (m, 3H), 3.43 (s, 3H), 3.20 (d, J=5.2 Hz, 3H), 3.14-2.81 (m, 2H), 2.37-2.16 (m, 1H), 2.13-1.70 (m, 2H), 1.56-1.31 (m, 2H), 1.01-0.83 (m, 3H), 0.77-0.55 (m 3H). ¹⁹F NMR (376 MHz, DMSO-d6, ppm): δ 88.66 (1F), 64.36 (4F).

### Example 25 Synthesis of Compound 84

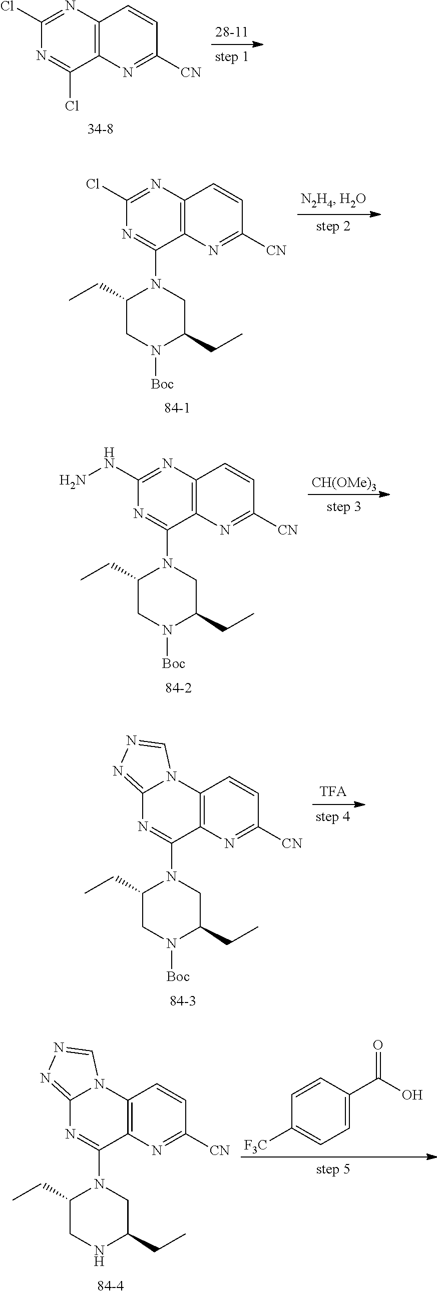

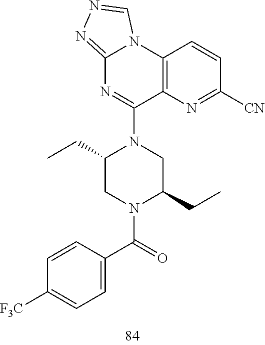 [SMILES: CC[C@@H]1CN(c2nc3nncn3c3ccc(C#N)nc23)[C@@H](CC)CN1C(=O)c1ccc(C(F)(F)F)cc1]

Step 1: To a solution of 34-8 (2.39 g, 10.62 mmol) in MeCN (50 mL) was added DIPEA (14.04 mL, 84.97 mmol) and 28-11 (2.57 g, 10.62 mmol) at room temperature. Then the mixture was stirred at room temperature for 1 h. The mixture was concentrated under reduced pressure. The residue was purified by column chromatography on silica gel (petroleum ether/ethyl acetate=1/0 to 10/1) to afford 84-1.

Step 2: To a solution of 84-1 (3.66 g, 8.49 mmol) in THF (30 mL) was added N₂H₄·H₂O (5 mL), the mixture was stirred at 50° C. for 1 h. The mixture was concentrated under reduced pressure. The residue was purified by column chromatography on silica gel (petroleum ether/ethyl acetate=10/0 to 0/1) to afford 84-2.

Step 3: 84-2 (3.6 g, 8.44 mmol) was dissolved in trimethoxymethane (30 mL), the mixture was stirred at 100° C. under N₂ for 16 h. The mixture concentrated under reduced pressure to afford product 84-3.

Step 4: To a solution of 84-3 (3.65 g, 8.36 mmol) in DCM (30 mL) was added TFA (10 mL) at room temperature and stirred for 5 h. The mixture was concentrated under reduced pressure, the residue was dissolved in DCM and washed with saturated NaHCO₃. The combined organic layer was dried over anhydrous Na₂SO₄, filtered and concentrated to afford 84-4.

Step 5: To a solution of 4-(trifluoromethyl)benzoic acid (100 mg, 0.53 mmol) in DMF (5 mL) was added DIPEA (0.13 mL, 0.79 mmol) and HATU (240.0 mg, 0.63 mmol), the mixture was stirred at 0° C. for 10 minutes, then 84-4 (176.9 mg, 0.53 mmol) was added to the mixture. The resulting mixture was stirred at room temperature for 16 h. The resulting mixture was purified by reverse phase HPLC (MeCN/water from 0/1 to 1/1) to afford 84. LCMS (ESI, m/z): [M+H]⁺=509.2; ¹H NMR (400 MHz, DMSO-d6, ppm) δ 9.55 (s, 1H), 9.02-8.88 (m, 1H), 8.67-8.50 (m, 1H), 7.93-7.81 (m, 2H), 7.74-7.59 (m, 2H), 5.87-4.23 (m, 3H), 3.78-3.42 (m, 2H), 3.38-3.12 (m, 1H), 2.03-1.54 (m, 4H), 1.11-0.88 (m, 3H), 0.77-0.50 (m, 3H). ¹⁹F NMR (377 MHz, DMSO-d6, ppm) 6-61.21 (3F).

### Example 26 Synthesis of Compound 91 and 92

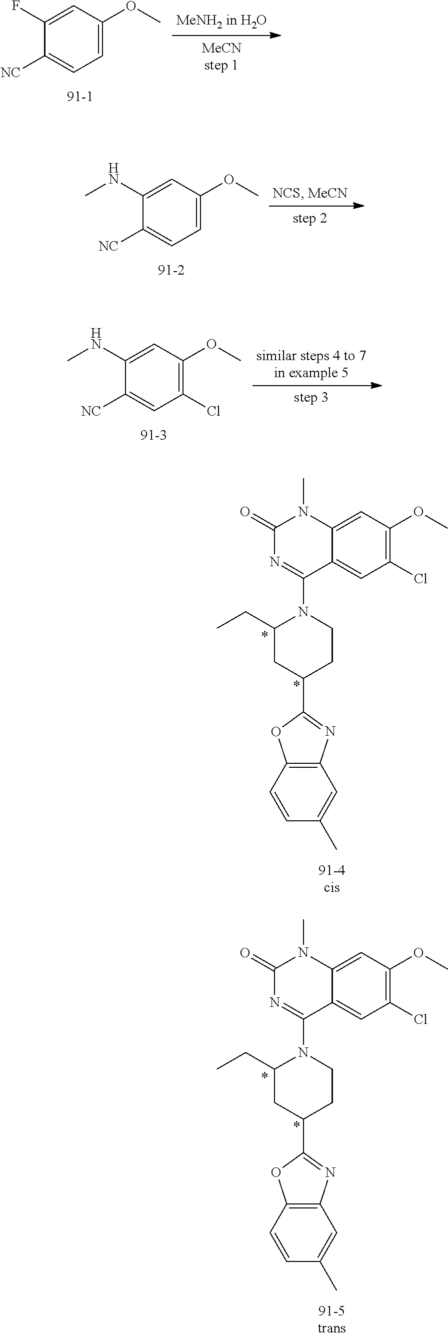

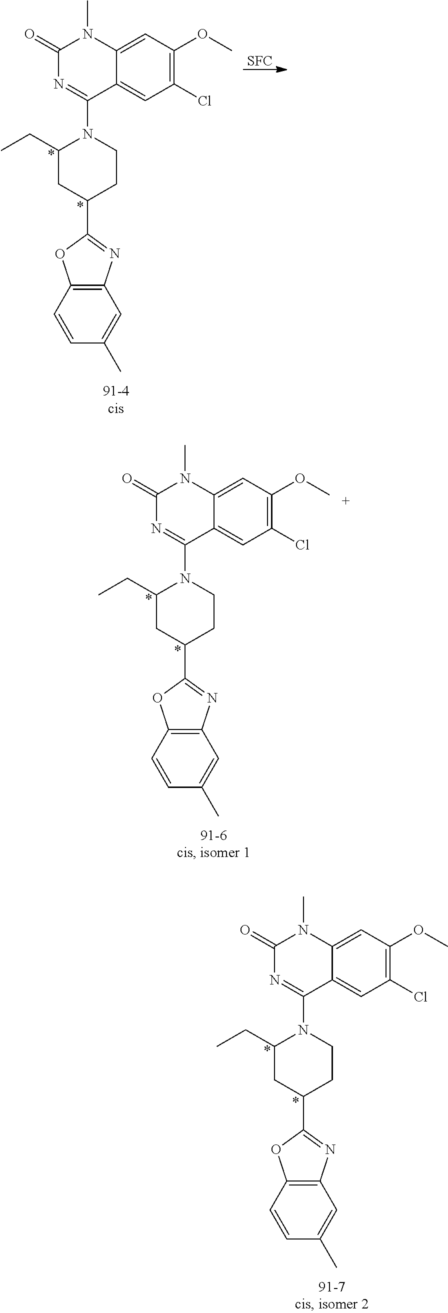

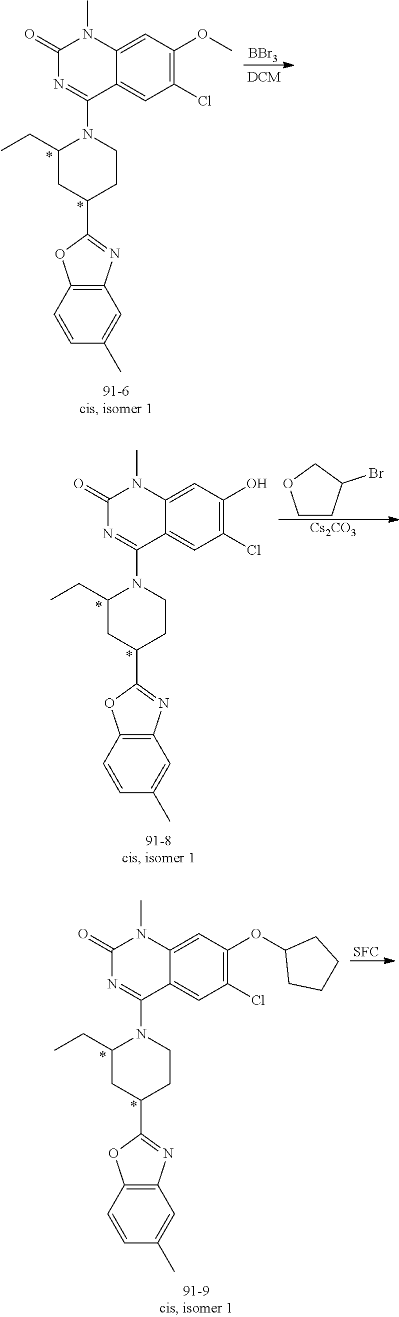

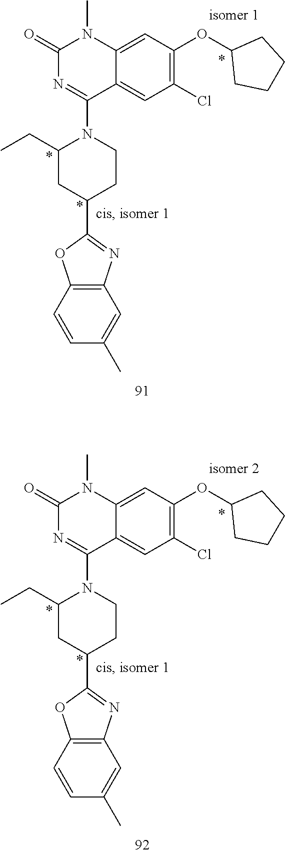 [SMILES: CCC1CC(c2nc3cc(C)ccc3o2)CCN1c1nc(=O)n(C)c2cc(OC3CCCC3)c(Cl)cc12 ; CCC1CC(c2nc3cc(C)ccc3o2)CCN1c1nc(=O)n(C)c2cc(OC3CCCC3)c(Cl)cc12]

Step 1: To a solution of 91-1 (15 g, 99.25 mmol) in MeCN (100 mL) was added CH₃NH₂ (200 mL, 3.56 mmol, 40% in H₂O). Then the reaction mixture was stirred at 80° C. for 16 h. The reaction mixture was quenched with H₂O. The mixture was extracted with ethyl acetate. The combined organic layers were washed with H₂O. The organic layer was dried over Na₂SO₄, filtered and concentrated to afford 91-2.

Step 2: To a solution of 91-2 (8.2 g, 50.56 mmol) in MeCN (150 mL) was added NCS (7.4 g, 55.61 mmol), then the reaction mixture was stirred at 60° C. for 70 minutes. The mixture was quenched by addition of water. The aqueous layer was extracted with ethyl acetate. The organic layer was combined, washed with brine, dried over anhydrous Na₂SO₄, filtered and concentrated. The residue was purified by column chromatography on silica gel (petroleum ether/ethyl acetate=1/0 to 5/1) to afford 92-3.

Step 3: Compound 91-4 and 91-5 was prepared from compound 91-3 following the procedure for the synthesis of compound 5-8 in example 5, the product was obtained by reverse phase HPLC (MeCN/water, 55%-65%).

Step 4: 91-4 (1.2 g) was purified by SFC (column: REGIS (S,S)WHELK-O1, 250×25 mm I.D., 10 μm, A for CO₂ and B for Ethanol) to afford 91-6 (200 mg) and 91-7 (250 mg) respectively. 91-6: SFC analysis: 100.00% ee; retention time: 5.932 min; column: ChiralPak AD, 250×30 mm I.D., 10 μm, A for CO₂ and B for Ethanol, 40%; pressure: 100 bar; flow rate: 70 mL/min. 91-7: SFC analysis: 98.76% ee; retention time: 7.760 min; column: ChiralPak AD, 250×30 mm I.D., 10 μm, A for CO₂ and B for Ethanol, 40%; pressure: 100 bar; flow rate: 70 mL/min.

Step 5: To a solution of 91-6 (180 mg, 0.39 mmol) in DCM (1 mL) under N₂ at −78° C. was added dropwise BBr₃ (3.86 mL, 3.86 mmol). After the addition was completed, the mixture was stirred at room temperature for 16 h. Ice water was added to the mixture to quench the reaction and the precipitation was collected by filtration, washed with H₂O and dried to afford 91-8.

Step 6: The mixture of 91-8 (160 mg, 0.35 mmol), 3-bromotetrahydrofuran (67.84 mg, 0.45 mmol) and Cs₂CO₃ (266.2 mg, 0.82 mmol) in DMF (5 mL) was stirred at 100° C. for 6 h. Then the mixture was washed with water and extracted with ethyl acetate. The organic layer was dried over Na₂SO₄, filtered and concentrated to afford 91-9.

Step 7: 91-9 (110 mg) was purified by SFC (column: ChiralPak AD, 250×30 mm ID., 10 μm, A for CO₂ and B for Ethanol) to afford 91 (26 mg) and 92 (15 mg) respectively. 91: SFC analysis: 100.00% ee; retention time: 2.519 min; column: ChiralPak AD, 250×30 mm I.D., 10 μm, A for CO2 and B for Ethanol, 35%; pressure: 100 bar; flow rate: 80 mL/min. LCMS (ESI, m/z): [M+H]⁺=523.4; ¹H NMR (400 MHz, DMSO-d6, ppm): δ 7.77 (s, 1H), 7.57 (d, J=8.4 Hz, 1H), 7.52 (s, 1H), 7.19 (d, J=8.4 Hz, 1H), 6.91 (s, 1H), 5.45-5.40 (m, 1H), 4.18-4.12 (m, 1H), 3.99-3.93 (m, 1H), 3.89-3.85 (m, 3H), 3.82-3.71 (m, 2H), 3.48 (s, 3H), 3.45-3.40 (m, 2H), 2.38-2.32 (m, 2H), 2.30-2.26 (m, 2H), 2.21-2.17 (m, 1H), 2.15-1.97 (m, 3H), 1.58-1.52 (m, 1H), 1.45-1.33 (m, 1H), 0.71 (t, J=7.6 Hz, 3H). 92: SFC analysis: 100.00% ee; retention time: 2.012 min; column: ChiralPak AD, 250×30 mm I.D., 10 μm, A for CO₂ and B for Ethanol, 35%; pressure: 100 bar; flow rate: 80 mL/min. LCMS (ESI, m/z): [M+H]⁺=523.4; ¹H NMR (400 MHz, DMSO-d6, ppm): δ 7.77 (s, 1H), 7.61-7.48 (m, 2H), 7.19 (d, J=8.0 Hz, 1H), 6.91 (s, 1H), 5.92-5.30 (m, 1H), 4.20-4.15 (m, 1H), 3.98-3.92 (m, 1H), 3.92-3.84 (m, 3H), 3.80-3.70 (m, 2H), 3.55-3.45 (m, 5H), 2.38-2.32 (m, 2H), 2.30-2.26 (m, 2H), 2.21-2.15 (m, 1H), 2.15-1.98 (m, 3H), 1.60-1.52 (m, 1H), 1.43-1.34 (m, 1H), 0.71 (t, J=7.0 Hz, 3H).

### Example 27 Synthesis of Compound 95

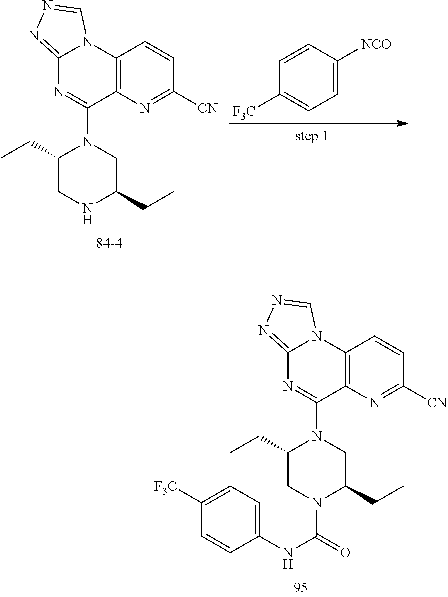

Step 1: To a solution of 84-4 (179.8 mg, 0.53 mmol) in DCM (2 mL) was added dropwise a solution of 1-isocyanato-4-(trifluoromethyl)benzene (100 mg, 0.53 mmol) in DCM (2 mL). The resulting mixture was stirred at room temperature for 16 h. The resulting mixture was concentrated, the residue was purified by column chromatography on silica gel (DCM/MeOH=10/1) to afford 95. LCMS (ESI, m/z): [M+H]⁺=524.2; ¹H NMR (400 MHz, DMSO-d6, ppm) δ 9.55 (s, 1H), 8.98-8.91 (m, 2H), 8.63-8.55 (m, 1H), 7.75-7.66 (m, 2H), 7.64-7.57 (m, 2H), 5.51-4.83 (m, 2H), 4.41 (m, 1H), 4.20-4.07 (m, 1H), 3.58 (m, 1H), 3.33-3.13 (m, 1H), 1.86-1.63 (m, 4H), 0.97-0.85 (m, 6H). ¹⁹F NMR (377 MHz, DMSO) δ −59.99 (s,3F).

### Example 28 Synthesis of Compound 98 and 99

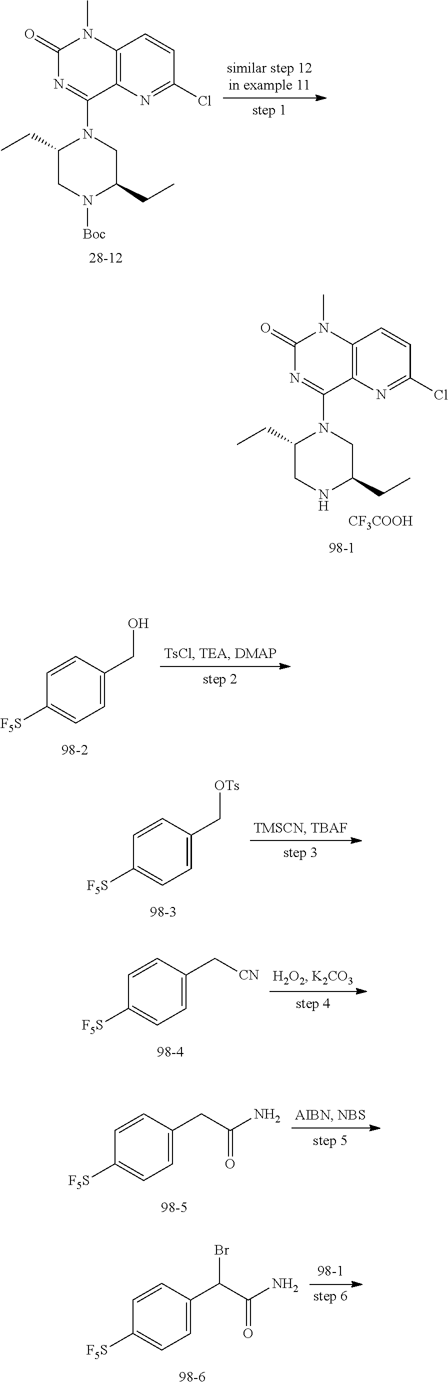

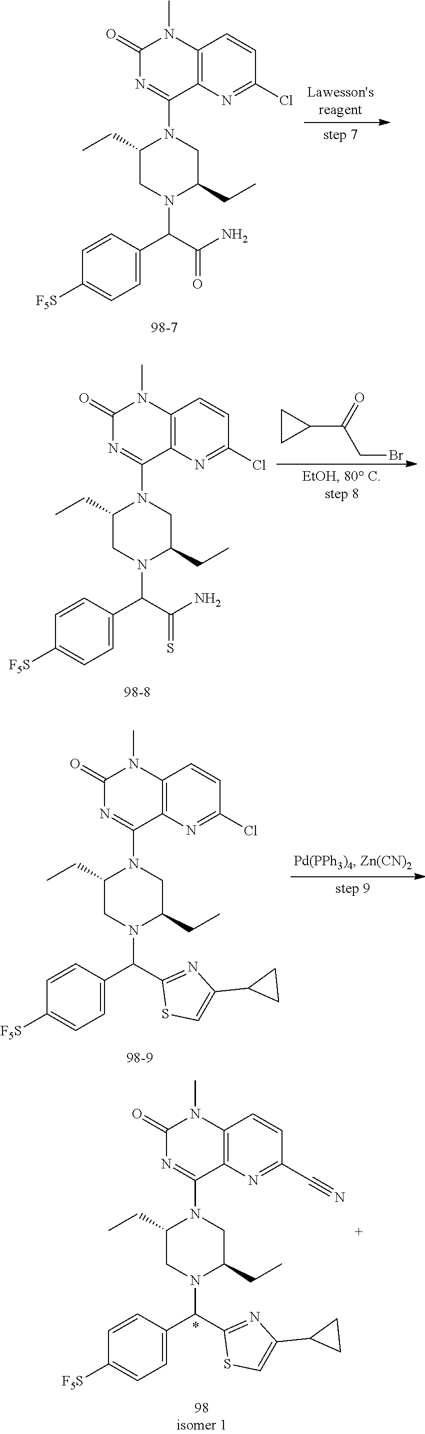

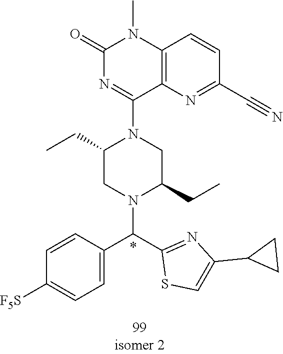

Step 1: Compound 98-1 was prepared from compound 28-12 following the procedure for the synthesis of compound 28-14 in example 11.

Step 2: To a 0° C., solution of 98-2 (4 g, 17.08 mmol) in DCM (100 mL) were added triethylamine (5.18 g, 51.24 mmol), DMAP (357.2 mg, 1.71 mmol) and 4-methylbenzenesulfonyl chloride (4.88 g, 25.62 mmol). Then the reaction mixture was stirred at room temperature for 4 h. The reaction was quenched with H₂O, extracted with DCM. The organic layer was dried over anhydrous Na₂SO₄, filtered and concentrated. The residue was purified by column chromatography on silica gel (petroleum ether/ethyl acetate=1/0 to 5/1) to afford 98-3.

Step 3: To a solution of 98-3 (3.8 g, 9.78 mmol) in THF (100 mL) were added TBAF (1M in THF, 14.7 mL, 14.7 mmol) and TMSCN (1.16 g, 11.74 mmol). The reaction mixture was stirred at 60° C. for 1 h. Then the reaction was quenched with H₂O, extracted with ethyl acetate. The organic layer was dried over anhydrous Na₂SO₄, filtered and concentrated. The residue was purified by column chromatography on silica gel (petroleum ether/ethyl acetate=1/0 to 4/1) to afford 98-4.

Step 4: To a solution of 98-4 (1 g, 4.11 mmol) in acetone (20 mL) was added K₂CO₃ (1.14 g, 8.22 mmol) and H₂O₂(4 mL). Then the mixture was stirred at 40° C. for 48 h. Then the reaction was quenched with H₂O, extracted with ethyl acetate. The organic layer was dried over anhydrous Na₂SO₄, filtered and concentrated. The residue was purified by column chromatography on silica gel (DCM/MeOH=1/0 to 10/1) to afford 98-5.

Step 5: To a solution of 98-5 (670 mg, 2.56 mmol) in CCl₄ (10 mL) were added AIBN (42 mg, 0.26 mmol) and NBS (683.44 mg, 3.84 mmol). Then the reaction mixture was stirred at 90° C. for 16 h. Then the reaction was quenched with H₂O, extracted with ethyl acetate. The organic layer was dried over anhydrous Na₂SO₄, filtered and concentrated. The residue was purified by column chromatography on silica gel (DCM/MeOH=1/0 to 10/1) to afford 98-6.

Step 6: To a solution of 98-6 (200 mg, 0.59 mmol) in MeCN (4 mL) were added 98-1 (265.5 mg, 0.59 mmol), DIPEA (380 mg, 2.94 mmol) and NaI (105.8 mg, 0.71 mmol). Then the reaction was stirred at 90° C. for 2 h. The solvent was removed under vacuum, and the residue was purified by reverse phase HPLC (MeCN/H₂O, 5-80%) to afford 98-7.

Step 7: To a solution of 98-7 (110 mg, 0.19 mmol) in THF (4 mL) was added Lawesson's reagent (74.83 mg, 0.19 mmol). Then the reaction mixture was stirred at 50° C. for 30 h. The solvent was removed under vacuum, and the residue was purified by reverse phase HPLC (MeCN/H₂O, 5-80%) to afford 98-8.

Step 8: To a solution of 98-8 (30 mg, 0.049 mmol) in EtOH (1 mL) was added 2-bromo-1-cyclopropylethan-1-one (16 mg, 0.098 mmol). Then the reaction mixture was stirred at 80° C. for 2 h. The solvent was removed under vacuum. The residue was purified by column chromatography on silica gel (petroleum ether/ethyl acetate=1/0 to 2/3) to afford 98-9.

Step 9: To a mixture of 98-9 (20 mg, 0.03 mmol) in DMF (1 mL) was added Zn(CN)₂ (10.6 mg, 0.09 mmol) and Pd(PPh₃)₄(3.5 mg, 0.003 mmol). Then the mixture was stirred at 130° C. for 2 h under microwave. The reaction mixture was purified by reverse phase HPLC (MeCN/H₂O (0.05% TFA), 5-80%) to afford 98 (5 mg) and 99 (7 mg). 98: LCMS (ESI, m/z): [M+H]⁺=666.4; ¹H NMR (400 MHz, DMSO-d₆, ppm): δ 8.23 (d, J=8.8 Hz, 1H), 8.01-7.88 (m, 3H), 7.79-7.70 (m, 2H), 7.25 (s, 1H), 5.96-5.82 (m, 0.5H), 5.42-5.38 (m, 0.5H), 5.22 (s, 1H), 4.98-4.83 (m, 1H), 3.62-3.58 (m, 0.5H), 3.43 (s, 3H), 3.38-3.32 (m, 0.5H), 2.80-2.71 (m, 1H), 2.23-2.18 (m, 1H), 2.05-1.88 (m, 3H), 1.49-1.38 (m, 3H), 0.86-0.77 (m, 6H), 0.70-0.64 (m, 1H), 0.62-0.45 (m, 3H). ¹⁹F NMR (376 MHz, DMSO-d₆, ppm): δ 87.47 (1F), 64.17 (4F). 99: LCMS (ESI, m/z): [M+H]⁺=666.4; ¹H NMR (400 MHz, DMSO-d₆, ppm): δ 8.28-8.19 (m, 1H), 8.02-7.88 (m, 3H), 7.82-7.73 (m, 2H), 7.30 (s, 1H), 5.95-5.82 (m, 0.5H), 5.43-5.38 (m, 0.5H), 5.27 (s, 1H), 5.01-4.85 (m, 1H), 3.62-3.58 (m, 0.5H), 3.49-3.42 (m, 3.5H), 2.20-2.11 (m, 1H), 2.08-1.84 (m, 4H), 1.73-1.37 (m, 3H), 0.90-0.82 (m, 2H), 0.79-0.61 (m, 8H). ¹⁹F NMR (376 MHz, DMSO-d₆, ppm): δ 87.33 (1F), 64.18 (4F).

### Example 29 Synthesis of Compound 102

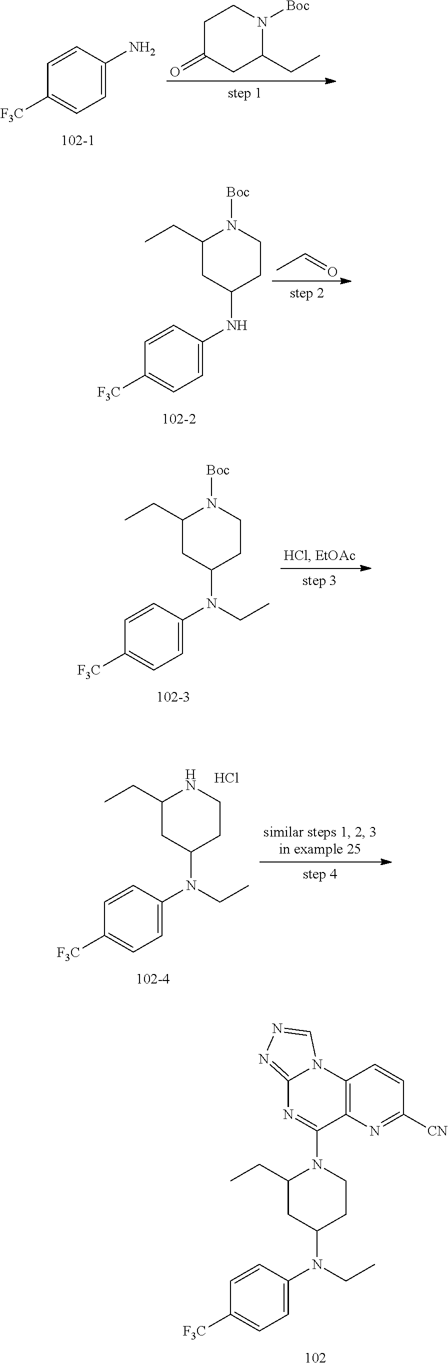

Step 1: To a mixture of 102-1 (400 mg, 2.48 mmol), tert-butyl 2-ethyl-4-oxopiperidine-1-carboxylate (564.3 mg, 2.48 mmol) in THF (10 mL) was added dropwise TiCl₄ (235.4 mg, 1.24 mmol) at room temperature. The mixture was stirred for another 30 minutes, then sodium triacetoxyborohydride (1052.3 mg, 4.97 mmol) was added and the mixture was stirred at room temperature for 12 h. The mixture was concentrated. The residue was purified by column chromatography on silica gel (petroleum ether/ethyl acetate=1/0 to 4/1) to afford 102-2.

Step 2: To a mixture of 102-2 (646 mg, 1.74 mmol), acetaldehyde (382.1 mg, 8.68 mmol) in THF (10 mL) was added dropwise TiCl₄ (164.5 mg, 0.87 mmol) at room temperature. The mixture was stirred for another 30 minutes, then and sodium triacetoxyborohydride (735.2 mg, 3.47 mmol) was added and the mixture was stirred at room temperature for 12 h. The reaction mixture concentrated. The residue was purified by column chromatography on silica gel (petroleum ether/ethyl acetate=10/1 to 3/1) to afford 102-3.

Step 3: To a solution of 102-3 (345 mg, 0.86 mmol) in DCM (5 mL) was added ethyl acetate/HCl (0.43 mL) and the mixture was stirred at room temperature for 2 h. The reaction mixture was concentrated to dryness to afford 102-4 which was used for the next step directly without further purification.

Step 4: Compound 102 was prepared from compound 102-4 following the procedure for the synthesis of compound 84-3 in example 25. LCMS (ESI, m/z): [M+H]⁺=495.3; ¹H NMR (400 MHz, DMSO-d6, ppm): 69.52 (s, 1H), 8.95-8.93 (d, J=8.8 Hz, 1H), 8.58-8.56 (d, J=8.8 Hz, 1H), 7.39-7.37 (d, J=8.8 Hz, 2H), 6.83-6.81 (d, J=8.8 Hz, 2H), 4.75-4.69 (m, 2H), 4.05-4.03 (m, 1H), 3.72-3.71 (m, 1H), 3.45-3.34 (m, 2H), 2.34-2.33 (m, 1H), 2.14-1.75 (m, 5H), 1.17-0.98 (m, 6H). ¹⁹F NMR (376 MHz, DMSO-d₆, ppm): δ −58.90 (3F).

### Example 30 Synthesis of Compound 103

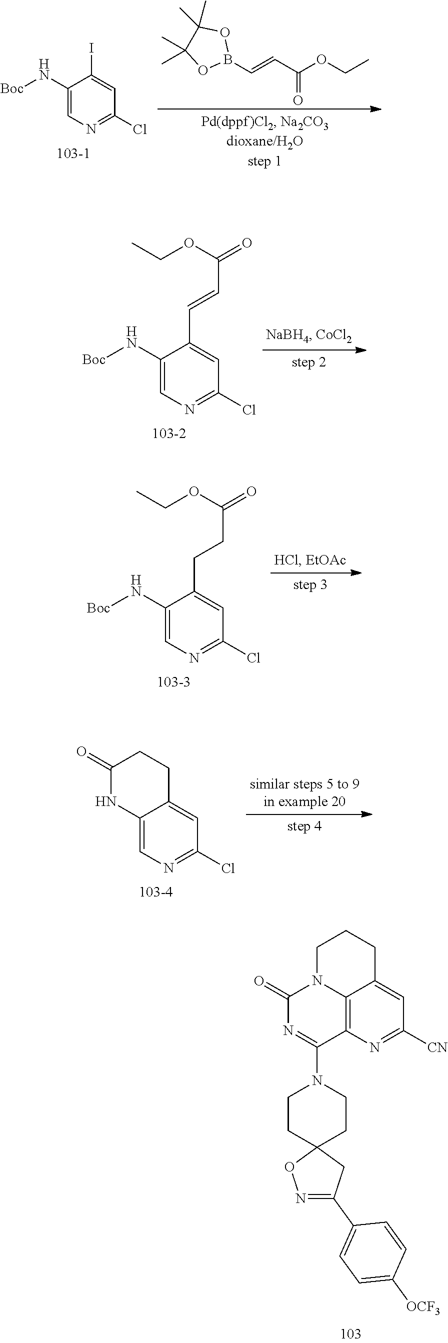 [SMILES: CC(C)(C)OC(=O)Nc1cnc(Cl)cc1I ; CCOC(=O)/C=C/B1OC(C)(C)C(C)(C)O1 ; CCOC(=O)/C=C/c1cc(Cl)ncc1NC(=O)OC(C)(C)C ; CCOC(=O)CCc1cc(Cl)ncc1NC(=O)OC(C)(C)C ; O=C1CCc2cc(Cl)ncc2N1 ; N#Cc1cc2c3c(n1)c(N1CCC4(CC1)CC(c1ccc(OC(F)(F)F)cc1)=NO4)nc(=O)n3CCC2]

Step 1: To a mixture of 103-1 (9.50 g, 26.79 mmol) and ethyl (2E)-3-(4,4,5,5-tetramethyl-1,3,2-dioxaborolan-2-yl)prop-2-enoate (7.87 g, 34.83 mmol) in dioxane (95 mL) and H₂O (9.5 mL) were added Pd(dppf)Cl₂ (2.0 g, 2.68 mmol) and Na₂CO₃ (5.7 g, 53.59 mmol) at room temperature. Then the mixture was heated at 100° C. for 12 h. The mixture was filtered and the filtrate was concentrated under vacuo. The residue was purified by column chromatography on silica gel (petroleum ether/ethyl acetate=1/0 to 1/4) to afford 103-2.

Step 2: To a mixture of 103-2 (12 g, 25.71 mmol) and CoCl₂·6H₂O (0.6 g, 2.57 mmol) in MeOH (360 mL) and THF (180 mL) was added NaBH₄ (5.8 g, 154.23 mmol) in portions at 0° C. Then the mixture was stirred at room temperature for 1 h. H₂O was added to the reaction mixture. This mixture was extracted with ethyl acetate. The combined organic layers were dried over Na₂SO₄, filtered and the filtrate was concentrated under vacuo. The residue was purified by column chromatography on silica gel (DCM) to afford 103-3.

Step 3: 103-3 (7.6 g, 23.12 mmol) in HCl/ethyl acetate (100 mL) was stirred at room temperature for 1 h. The mixture was concentrated under vacuo to afford 103-4.

Step 4: Compound 103 was prepared from compound 103-4 following the procedure for the synthesis of compound 59 in example 20. LCMS (ESI, m/z): [M+H]⁺=511.2; ¹H NMR (400 MHz, DMSO-d6, ppm): δ 8.07 (s, 1H), 7.82-7.77 (m, 2H), 7.49-7.47 (m, 2H), 4.92-4.17 (m, 4H), 3.91-3.83 (m, 2H), 3.35 (s, 2H), 2.96-2.89 (m, 2H), 2.02-1.90 (m, 6H). ¹⁹F NMR (376 MHz, DMSO-d₆, ppm): δ −56.75 (3F).

### Example 31 Synthesis of Compound 104

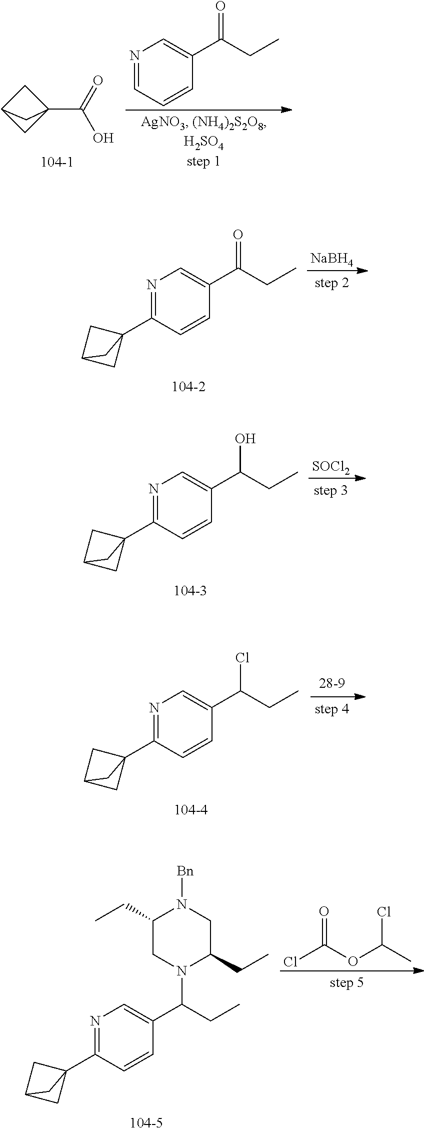

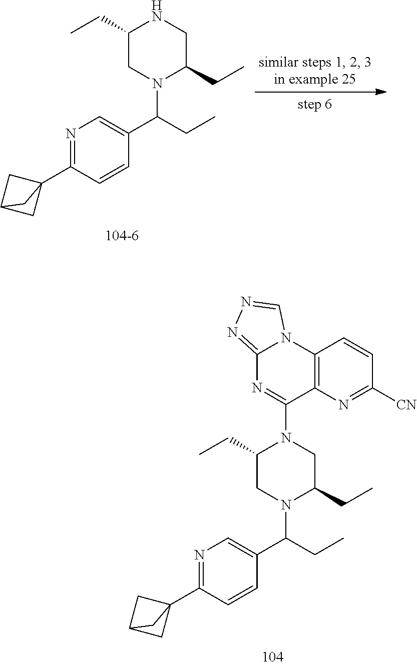 [SMILES: CCC(c1ccc(C23CC(C2)C3)nc1)N1C[C@H](CC)NC[C@H]1CC ; CCC(c1ccc(C23CC(C2)C3)nc1)N1C[C@H](CC)N(c2nc3nncn3c3ccc(C#N)nc23)C[C@H]1CC]

Step 1: The mixture of 104-1 (5.0 g, 44.59 mmol), 1-(pyridin-3-yl)propan-1-one (7.23 g, 53.51 mmol), AgNO₃ (2.27 g, 13.38 mmol), H₂SO₄ (9.75 g, 89.18 mmol) and ammonium persulfate (10.17 g, 44.59 mmol) in H₂O (70 mL) and DCM (70 mL) was flushed with N₂ for 0.5 min, then the mixture was stirred at 25° C. for 18 h. The mixture quenched by addition of saturated NaHCO₃ solution, the aqueous layer was extracted with DCM. The organic extracts were combined, washed with brine, dried over anhydrous Na₂SO₄. The solvent was concentrated and the residue was purified by column chromatography on silica gel (petroleum ether/ethyl acetate=3/1) to afford 104-2.

Step 2: To a solution of 104-2 (560 mg, 2.78 mmol) in MeOH(10 mL) was added NaBH₄ (12.3 mg, 3.34 mmol) at 0° C., the reaction mixture was stirred at room temperature for 2 h. The mixture was quenched by addition of H₂O, and the aqueous layer was extracted with ethyl acetate. The organic extracts were combined, washed with brine, dried over anhydrous Na₂SO₄ and concentrated to afford 104-3.

Step 3: To a solution of 104-3 (580 mg, 2.85 mmol) in DCM (15 mL) were added DMF (21.0 mg, 0.29 mmol) and SOCl₂ (509 mg, 4.28 mmol) at 0° C., the reaction mixture was stirred at room temperature for 3 hr. The solvent was removed under reduced pressure to afford 104-4 which was used for next step directly without further purification.

Step 4: The mixture of 28-9 (100 mg, 0.43 mmol), 104-4 (239 mg, 1.08 mmol), DIPEA (0.29 mL, 1.72 mmol) and NaI (129 mg, 0.86 mmol) in MeCN (2 mL) was stirred at 85° C. for 18 h. The mixture was filtered and concentrated, the residue was purified by reverse phase HPLC (0.05% NH₄HCO₃ in water/MeCN, 5-95%) to afford 104-5.

Step 5: To a solution of 104-5 (80 mg, 0.19 mmol) in 1,2-dichloroethane (1 mL) was added 1-chloroethyl chloromethanoate (274.5 mg, 1.92 mmol), the reaction was stirred at 110° C. for 96 h. The mixture was concentrated in vacuo, the residue was diluted with THF and H₂O. Then the mixture was stirred at 70° C. for 3 h. The solvent was removed under reduced pressure to afford 104-6 which was used for next step directly without further purification.

Step 6: Compound 104 was prepared from compound 104-6 following the procedure for the synthesis of compound 84-3 in example 25. LCMS (ESI, m/z): [M+H]⁺=522.4. ¹H NMR (400 MHz, DMSO-d₆, ppm) δ 9.49-9.46 (m, 1H), 8.92-8.87 (m, 1H), 8.56-8.51 (m, 1H), 8.42-8.37 (m, 1H), 7.73-7.67 (m, 1H), 7.30-7.23 (m, 1H), 5.94-5.83 (m, 0.5H), 5.45-5.23 (m, 0.5H), 5.21-4.74 (m, 1H), 3.64-3.47 (m, 1H), 3.46-3.40 (m, 0.5H), 3.30-3.07 (m, 0.5H), 2.92-2.80 (m, 1H), 2.56-2.55 (m, 1H), 2.30-2.23 (m, 1H), 2.14-2.08 (m, 6H), 2.00-1.82 (m, 3H), 1.70-1.35 (m, 4H), 1.03-0.94 (m, 3H), 0.72-0.58 (m, 6H).

### Example 32 Synthesis of Compound 107 and 108

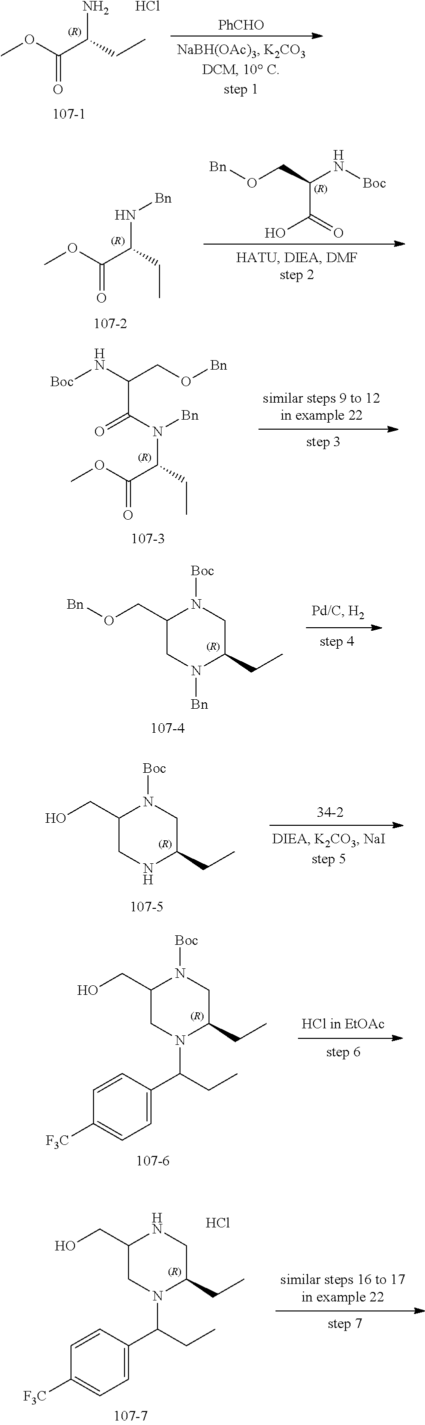

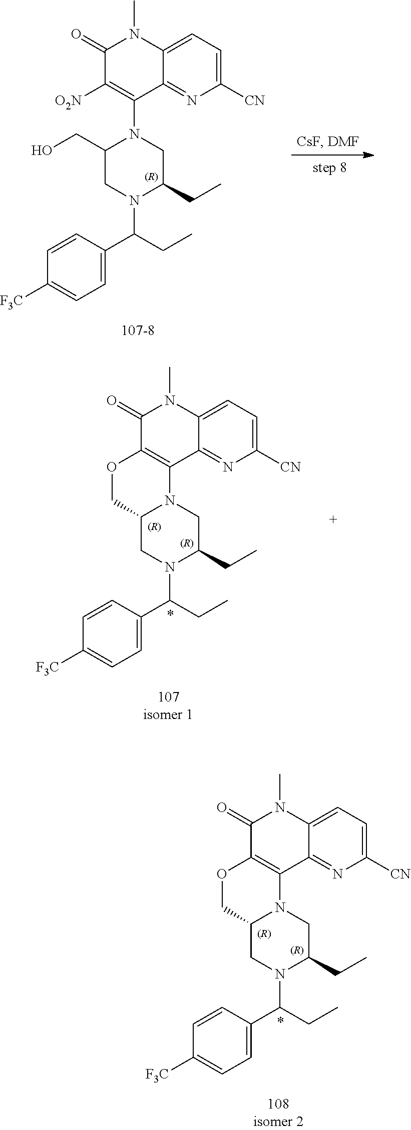

Step 1: To a stirred solution of 107-1 (500 g, 3.27 mol) in dry DCM (5000 mL) was added benzaldehyde (352 g, 3.32 mol), K₂CO₃ (494.9 g, 3.58 mol) and Na₂SO₄(462 g, 3.25 mol). The reaction mixture was stirred at room temperature for 6 h. Then sodium triacetoxyborohydride (1030 g, 4.86 mol) was added in portion wise over 30 minutes to the mixture at ice-bath. The mixture was stirred at room temperature for 16 h. The solid was removed by filtration and the solvent was removed under reduced pressure. The residue was partitioned between ethyl acetate and HCl (1N). The mixture was extracted with ethyl acetate. The pH of the aqueous layer was adjusted to 8.0 with NaHCO₃(aq) and the milky aqueous layer was extracted immediately with ethyl acetate. The combined organic layer was washed with brine, dried over anhydrous Na₂SO₄, filtered and concentrated under reduced pressure to afford 107-2.

Step 2: To a solution of 107-2 in DMF (3500 mL) were added DIPEA (885 mL, 5.08 mol) and HATU (966 g, 2.54 mol), the reaction mixture was stirred at 0° C. for 10 minutes, then methyl (2R)-2-(benzylamino)butanoate (351 g, 1.69 mol) was added to the reaction mixture. The result mixture was stirred at room temperature for 16 h. The reaction mixture was diluted with ethyl acetate and washed with water. The organic layer was dried over anhydrous Na₂SO₄, filtered and evaporated under reduced pressure to afford 107-3.

Step 3: Compound 107-4 was prepared from compound 107-3 following the procedure for the synthesis of compound 67-14 in example 22.

Step 4: To a stirred solution of 107-4 (4.8 g, 11.31 mmol) in isopropanol (150 mL) was added Pd/C (10%, 0.96 g, 9.02 mmol) at room temperature, the resulting mixture was stirred at 80° C. under H₂ balloon for 15 h. The reaction mixture was filtered through a pad of celite and the filtrate was concentrated to afford 107-5.

Step 5: A mixture of 107-5 (1 g, 3.89 mmol), 34-2 (1.73 g, 7.78 mmol), NaI (1.2 g, 7.78 mmol), DIPEA (2.03 mL, 11.66 mmol) and K₂CO₃ (6.1 g, 44.2 mmol) in CH₃CN (15 mL) was stirred at 85° C. for 15 h. Another portion of DIPEA (2.03 mL, 11.66 mmol), NaI (1.2 g, 7.78 mmol) and 34-2 (1.73 g, 7.78 mmol) was added into the reaction mixture at 20° C. The resulting mixture was stirred at 85° C. under N₂ for 15 h. The reaction mixture was concentrated. The residue was purified by column chromatography on silica gel (petroleum ether/ethyl acetate=1/0 to 2/3) to afford 107-6.

Step 6: To a stirred solution of 107-6 (4.57 g, 10.09 mmol) in MeOH (20 mL) was added dropwise HCl in ethyl acetate (4M, 9.1 mL) at room temperature, the resulting mixture was stirred at 25° C. for 15 h. The reaction mixture was concentrated under reduced pressure to afford 107-7.

Step 7: Compound 107-8 was prepared from compound 107-7 following the procedure for the synthesis of compound 67-20 in example 22.

Step 8: To a stirred solution of 107-8 (350 mg, 0.63 mmol) in DMF (10 mL) was added CsF (285.5 mg, 1.88 mmol) at 15° C., the resulting mixture was stirred at 110° C. for 15 h. The reaction mixture was diluted with H₂O and extracted with ethyl acetate. The organic layer was combined, washed with H₂O and brine, dried over anhydrous Na₂SO₄ and concentrated. The residue was purified by column chromatography on silica gel (petroleum ether/ethyl acetate=1/0 to 0/1) to afford 107 (60 mg) and 108 (crude, 110 mg), the crude 108 was purification again by reverse phase HPLC (MeCN/water (0.5% FA): 30/70) to afford pure 108 (44 mg) as 0.33 FA salt. 107: LCMS (ESI, m/z): [M+H]⁺=512.4. ¹H NMR (400 MHz, Methanol-d₄, ppm) δ 8.04 (d, J=8.7 Hz, 1H), 7.89 (d, J=8.7 Hz, 1H), 7.69-7.62 (m, 2H), 7.61-7.54 (m, 2H), 4.36-4.25 (m, 1H), 4.09-4.03 (m, 1H), 4.02-3.92 (m, 1H), 3.87-3.78 (m, 1H), 3.79-3.70 (m, 4H), 3.10-2.97 (m, 2H), 2.96-2.85 (m, 1H), 2.50-2.40 (m, 1H), 2.20-2.05 (m, 1H), 1.98-1.85 (m, 1H), 1.80-1.65 (m, 2H), 1.22 (t, J=7.4 Hz, 3H), 0.72 (t, J=7.3 Hz, 3H). ¹⁹F NMR (376 MHz, DMSO-d₆, ppm): δ −63.83 (3F). 108: LCMS (ESI, m/z): [M+H]⁺=512.4. ¹H NMR (400 MHz, Methanol-d₄, ppm) δ 8.01 (s, 0.33H), 8.00-7.98 (m, 1H), 7.86-7.83 (m, 1H), 7.65-7.63 (m, 2H), 7.48-7.46 (m, 2H), 4.72-4.60 (m, 1H), 4.40-4.32 (m, 1H), 4.15-4.00 (m, 2H), 3.68 (s, 3H), 3.45-3.38 (m, 1H), 3.30-3.26 (m, 1H), 3.23-3.16 (m, 1H), 2.38-2.37 (m, 1H), 2.26-2.20 (m, 1H), 2.10-1.92 (m, 2H), 1.90-1.70 (m, 2H), 1.03 (t, J=7.4 Hz, 3H), 0.87 (t, J=7.3 Hz, 3H). ¹⁹F NMR (376 MHz, DMSO-d₆, ppm): δ −63.91 (3F)

### Example 33 Synthesis of Compound 109 and 110

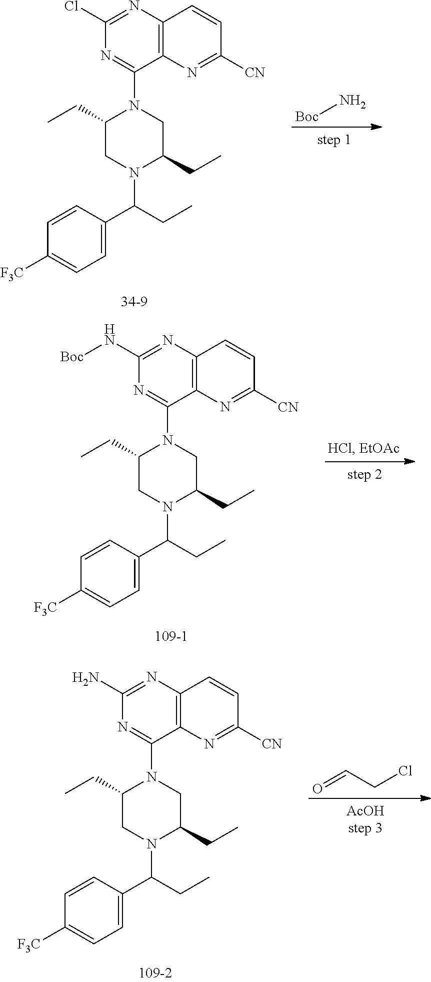

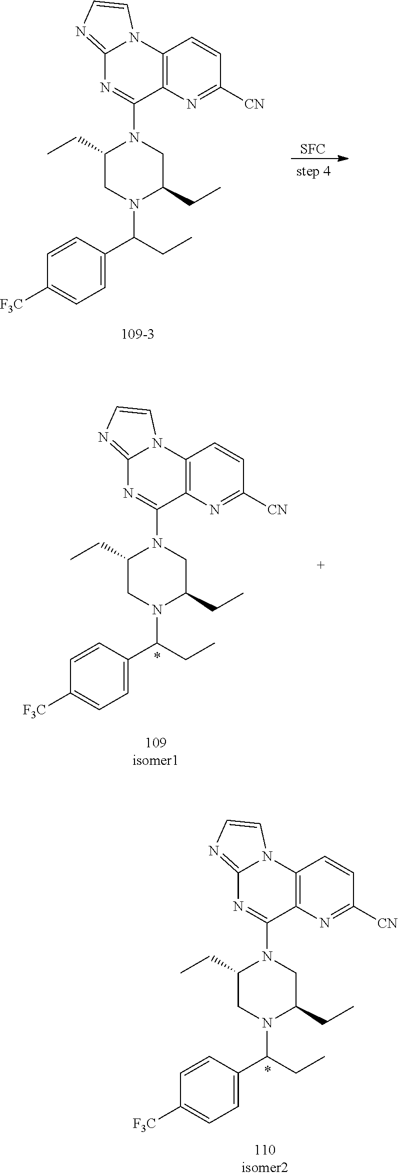 [SMILES: CCC(c1ccc(C(F)(F)F)cc1)N1C[C@H](CC)N(c2nc3nccn3c3ccc(C#N)nc23)C[C@H]1CC ; "SFC" ; CCC(c1ccc(C(F)(F)F)cc1)N1C[C@H](CC)N(c2nc3nccn3c3ccc(C#N)nc23)C[C@H]1CC ; CCC(c1ccc(C(F)(F)F)cc1)N1C[C@H](CC)N(c2nc3nccn3c3ccc(C#N)nc23)C[C@H]1CC]

Step 1: A solution of 34-9 (845 mg, 1.63 mmol), tert-butyl carbamate (287.2 mg, 2.45 mmol), Cs₂CO₃ (1.06 g, 3.27 mmol) and XantPhos Pd G₂ (17.2 mg, 0.019 mmol) in dioxane (15 mL) was stirred at 100° C. under N₂ for 6 h. The mixture was concentrated. The residue was purified by column chromatography on silica gel (petroleum ether/ethyl acetate=1/0 to 3/1) to afford 109-1.

Step 2: To a solution of 109-1 (660 mg, 1.1 mmol) in DCM (8 mL) was added HCl/ethyl acetate (6 mL). The mixture was stirred at 30° C. for 12 h. The mixture was concentrated to afford 109-2.

Step 3: A solution of 109-2 (611 mg, 1.23 mmol), 2-chloroacetaldehyde (5 mL, 0.49 mmol) and AcOH (0.070 mL, 1.23 mmol) in EtOH (12 mL) was stirred at 80° C. for 16 h. The mixture was concentrated. The residue was purified by reverse phase HPLC (MeCN/water (0.5% FA): 5%-80%) to afford 109-3.

Step 4: 109-3 (80 mg) was purified by prep-SFC (column: ChiralPak AD, 250×30 mm I.D., 5 μm, Ethanol/CO₂=40/60) to afford 109 (42 mg) and 110 (17 mg). 109: SFC analysis: 100% ee; retention time: 1.393 min; column: Chiralpak AD-3 150×4.6 mm I.D., 3 μm, 40% of Ethanol (0.05% of DEA) in C02; pressure: 100 bar; flow rate: 2.5 mL/min; LCMS (ESI, m/z): [M+H]⁺=522.3; ¹H NMR (400 MHz, DMSO-d₆, ppm) δ 8.84-8.82 (d, J=8.8 Hz, 1H), 8.47-8.45 (d, J=8.8 Hz, 1H), 8.184-8.181 (d, J=1.2 Hz, 1H), 7.75-7.73 (d, J=8.0 Hz, 2H), 7.59-7.57 (d, J=8.0 Hz, 2H), 7.328-7.325 (d, J=1.2 Hz, 1H), 5.45-4.80 (m, 2H), 3.73-3.54 (m, 1H), 3.35-3.26 (m, 1H), 3.01-2.91 (m, 1H), 2.88-2.80 (m, 1H), 2.36-2.31 (m, 1H), 2.12-1.88 (m, 3H), 1.67-1.39 (m, 3H), 1.03-0.86 (m, 3H), 0.71-0.52 (m, 6H). ¹⁹F NMR (377 MHz, DMSO-d₆, ppm) δ −60.66 (3F). 110: SFC analysis: 100% ee; retention time: 3.046 min; column: Chiralpak AD-3 150×4.6 mm I.D., 3 μm, 40% of Ethanol (0.05% of DEA) in C₀₂; pressure: 100 bar; flow rate: 2.5 mL/min; LCMS (ESI, m/z): [M+H]⁺=522.4; ¹H NMR (400 MHz, DMSO-d₆, ppm) δ 8.86-8.84 (d, J=8.8 Hz, 1H), 8.49-8.47 (d, J=8.8 Hz, 1H), 8.26-8.14 (m, 1H), 7.73-7.71 (d, J=8.0 Hz, 2H), 7.60-7.58 (d, J=8.0 Hz, 2H), 7.42-7.26 (m, 1H), 5.75-4.55 (m, 2H), 3.61-3.41 (m, 2H), 3.22-3.02 (m, 1H), 2.66-2.53 (m, 1H), 2.27-2.12 (m, 1H), 2.03-1.73 (m, 3H), 1.69-1.36 (m, 3H), 1.06-0.90 (m, 3H), 0.71-0.49 (m, 6H). ¹⁹F NMR (377 MHz, DMSO-d₆, ppm) δ −60.67 (3F).

### Example 34 Synthesis of Compound 113 and 114

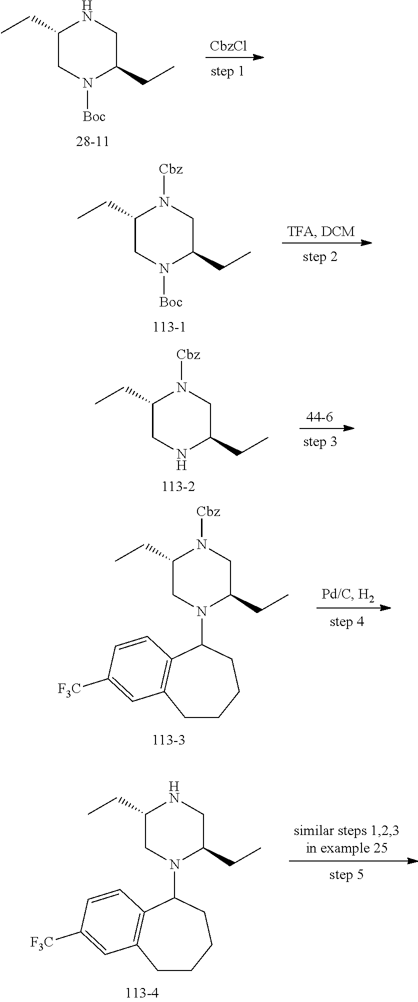

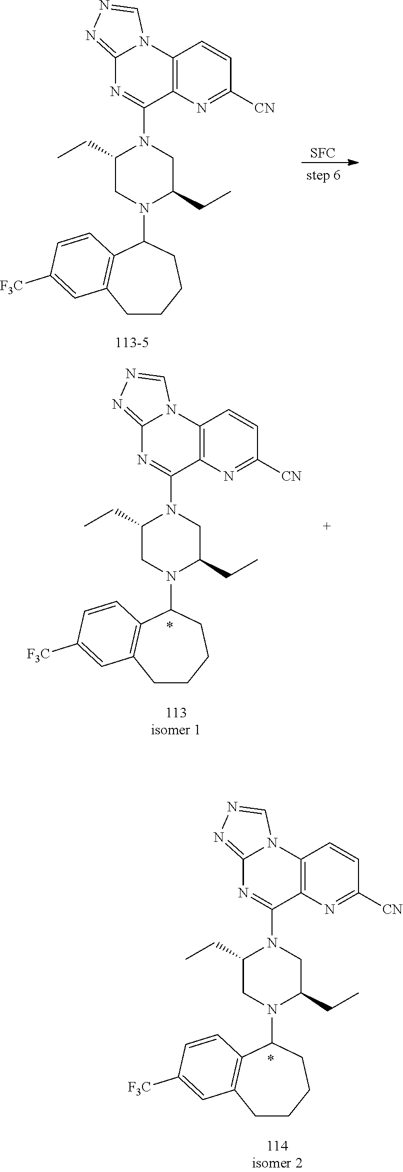 [SMILES: CC[C@H]1CN(C2CCCCc3cc(C(F)(F)F)ccc32)[C@H](CC)CN1c1nc2nncn2c2ccc(C#N)nc12 ; "SFC" ; CC[C@H]1CN(C2CCCCc3cc(C(F)(F)F)ccc32)[C@H](CC)CN1c1nc2nncn2c2ccc(C#N)nc12 ; CC[C@H]1CN(C2CCCCc3cc(C(F)(F)F)ccc32)[C@H](CC)CN1c1nc2nncn2c2ccc(C#N)nc12]

Step 1: To a solution of 28-11 (3.0 g, 12.38 mmol) in DCM (30 mL) was added DIPEA (5.12 mL, 30.95 mmol) and benzyl carbonochloridate (3.49 mL, 24.76 mmol), then the reaction mixture was stirred at 20° C. for 18 h. The reaction was concentrated in vacuo. The residue was diluted with ethyl acetate and water. The mixture was extracted with ethyl acetate. The combined organic layer was washed with NaCl aqueous and dried by Na₂SO₄, the solvent was concentrated and the residue was purified by column chromatography on silica gel (petroleum ether/ethyl acetate=1/0 to 3/1) to afford 113-1.

Step 2: To a solution of 113-1 (4.5 g, 11.98 mmol) in DCM (30 mL) was added TFA (6 mL, 11.98 mmol) and the reaction was stirred at 20° C. for 18 h. The reaction mixture was concentrated under vacuo, the residue was dilute with DCM and neutralized by NaHCO₃ aqueous. The mixture was extracted with DCM. The combined organic layer was concentrated to afford 113-2.

Step 3: To a solution of 113-2 (500 mg, 1.81 mmol) in MeCN (20 mL) was added DIPEA (0.6 mL, 3.62 mmol), NaI (543 mg, 3.62 mmol) and 44-6 (900 mg, 3.62 mmol), and the reaction mixture was stirred at 80° C. for 24 h. The reaction was concentrated in vacuo. The residue was diluted with ethyl acetate and water. The mixture was extracted with ethyl acetate. The combined organic layer was washed with NaCl aqueous and dried by Na₂SO₄, filtered and the solvent was concentrated. The residue was purified by column chromatography on silica gel (petroleum ether/ethyl acetate=1/0 to 1/1) to afford 113-3.

Step 4: To a solution of 113-4(280 mg, 0.57 mmol) in MeOH (10 mL) was added Pd/C 10% (6.1 mg, 0.057 mmol) under N₂. The then the reaction was stirred at 25° C. for 18 h at H₂ atmosphere. The mixture was filtered and the filtrate was concentrated to afford 113-4.

Step 5: Compound 113-5 was prepared from compound 113-4 following the procedure for the synthesis of compound 84-3 in example 25.

Step 6: 113-5 (73 mg) was purified by prep-SFC (column: ChiralPak IG, 250×30 mm I.D., 10 μm, MeOH(0.1% NH₃H2O)/CO2=40/60) to afford 113 (34.8 mg) and 114 (7.68 mg). 113: SFC analysis: 95% ee; retention time: 5.52 min; column: ChiralPak IG, 100×4.6 mm I.D., 3 μm, MeOH (0.05% DEA) in CO2, 5% to 40%; pressure: 100 bar; flow rate: 2.5 mL/min. LCMS (ESI, m/z): [M+H]⁺=549.4. ¹H NMR (400 MHz, DMSO-d₆, ppm): δ 9.50 (s, 1H), 8.93-8.91 (d, J=8.4 Hz, 1H), 8.56-8.54 (d, J=8.4 Hz, 1H), 7.53-7.40 (m, 3H), 6.18-4.42 (m, 2H), 3.86-3.77 (m, 1H), 3.69-3.40 (m, 2H), 3.04 (m, 1H), 2.83-2.71 (m, 1H), 2.62-2.53 (m, 1H), 2.15-1.90 (m, 5H), 1.87-1.71 (m, 2H), 1.65-1.48 (m, 3H), 1.39-1.27 (m, 1H), 1.06-0.97 (m, 3H), 0.68-0.51 (m, 3H). ¹⁹F NMR (376 MHz, DMSO-d₆, ppm) δ −60.77 (3F). 114: SFC analysis: 100% ee; retention time: 5.82 min; column: ChiralPak IG, 100×4.6 mm I.D., 3 μm, MeOH (0.05% DEA) in CO2, 5% to 40%; pressure: 100 bar; flow rate: 2.5 mL/min. LCMS (ESI, m/z): [M+H]⁺=549.4. ¹H NMR (400 MHz, methanol-d₄, ppm): δ 9.33 (s, 1H), 8.80-8.74 (m, 1H), 8.35-8.25 (m, 1H), 7.43-7.35 (m, 3H), 6.00-5.65 (m, 1H), 5.15-4.89 (m, 1H), 3.82-3.77 (m, 1H), 3.73-3.63 (m, 1H), 3.06-3.00 (m, 1H), 2.78-2.67 (m, 2H), 2.36-2.03 (m, 6H), 1.78-1.70 (m, 1H), 1.67-1.50 (m, 3H), 1.44-1.29 (m, 2H), 1.16-1.08 (m, 3H), 0.68-0.51 (m, 3H). ¹⁹F NMR (376 MHz, methanol-d₄, ppm): δ −63.91 (3F).

### Example 35 Synthesis of Compound 119 and 120

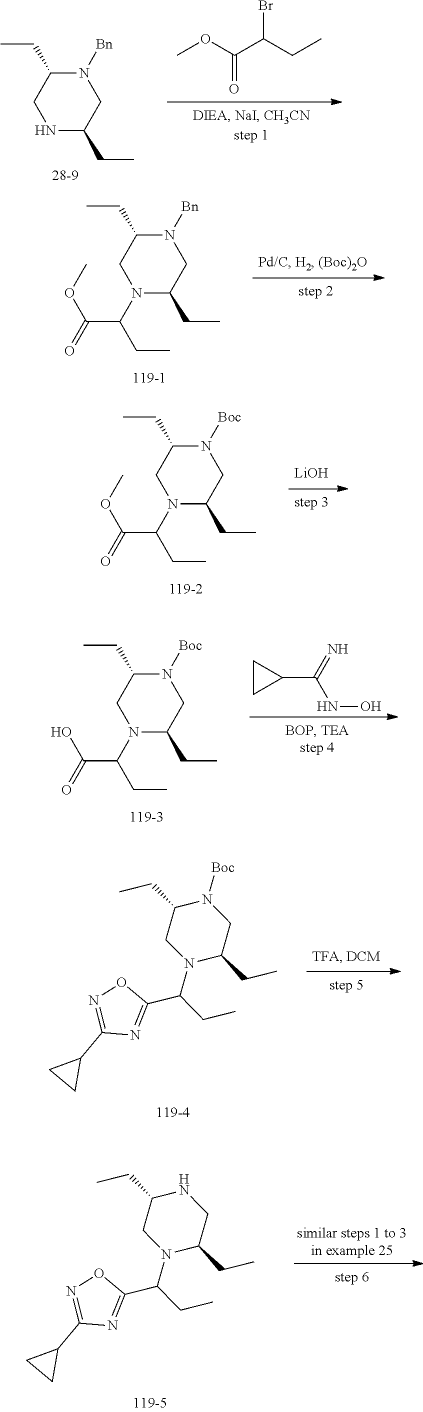

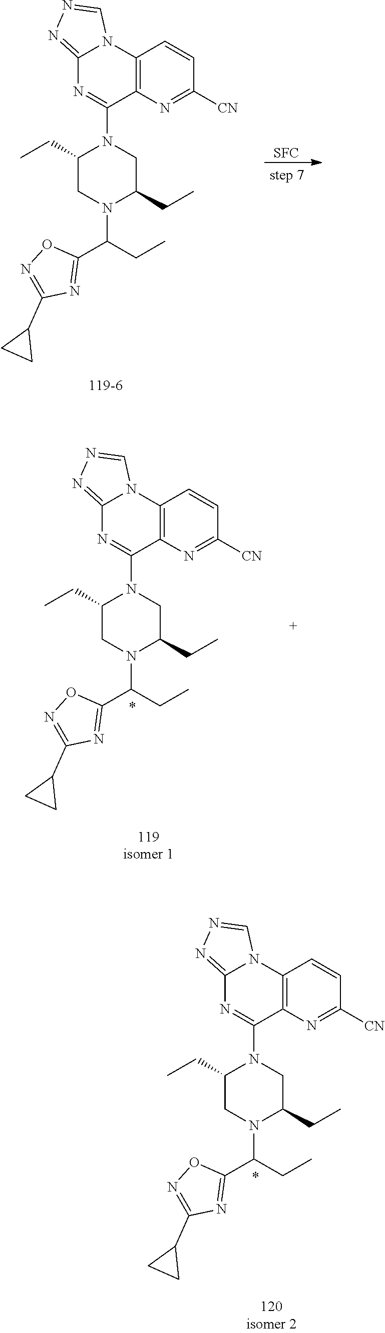 [SMILES: CCC(c1nc(C2CC2)no1)N1C[C@H](CC)N(c2nc3nncn3c3ccc(C#N)nc23)C[C@H]1CC ; "SFC" ; CCC(c1nc(C2CC2)no1)N1C[C@H](CC)N(c2nc3nncn3c3ccc(C#N)nc23)C[C@H]1CC ; CCC(c1nc(C2CC2)no1)N1C[C@H](CC)N(c2nc3nncn3c3ccc(C#N)nc23)C[C@H]1CC]

Step 1: To a solution of 28-9 (3 g, 12.91 mmol) and methyl 2-bromobutanoate (7.01 g, 38.73 mmol) in MeCN (50 mL) were added DIPEA (6.40 mL, 38.73 mmol) and NaI (1.9 g, 12.91 mmol) at room temperature. Then the mixture was heated at 80° C. for 12 h. The mixture was concentrated under vacuo, the residue was purified by column chromatography on silica gel (petroleum ether/ethyl acetate=1/0 to 10/1) to afford 119-1.

Step 2: To a solution of 119-1 (3.4 g, 10.23 mmol) in propan-2-ol (80 mL) was added (Boc)₂O (3.05 mL, 13.29 mmol) and Pd/C (10%, 0.5 g, 4.67 mmol) at room temperature. Then the mixture was stirred at 30° C. under H₂ balloon for 12 h. The mixture was filtered and the filtrate was concentrated under vacuo. The residue was purified by column chromatography on silica gel (petroleum ether/ethyl acetate=1/0 to 6/1) to afford 119-2.

Step 3: To a solution of 119-2 (3.0 g, 8.76 mmol) in THF (30 mL) and H₂O (20 mL) was added LiGH (0.6 g, 13.14 mmol) at room temperature. Then the mixture was stirred at 30° C. for 12 h. NaOH (1.1 g, 26.28 mmol) was added to the reaction mixture. The mixture was heated at 80° C. for 12 h. The solvent was removed under vacuo. The pH of the residue was adjusted to 3 with HCl(6N). The mixture was extracted with DCM. The combined organic layers were dried over Na₂SO₄, filtered and the filtrate was concentrated to afford 119-3.

Step 4: To a mixture of 119-3 (2.7 g, 8.22 mmol) and N-hydroxycyclopropanecarboximidamide (0.82 g, 8.22 mmol) in DMF (80 mL) was added BOP (10.9 g, 24.66 mmol) and triethylamine (3.43 mL, 24.66 mmol) at room temperature. Then the mixture was stirred at room temperature for 0.5 h and heated at 90° C. for 4 h. The mixture was concentrated under vacuo. The residue was purified by column chromatography on silica gel (petroleum ether/ethyl acetate=1/0 to 1/1) to afford 119-4.

Step 5: To a solution of 119-4 (400 mg, 1.02 mmol) in DCM (4 mL) was added TFA (4 mL) at room temperature. Then the mixture was stirred at room temperature for 1 h. The mixture was concentrated under vacuo to afford 119-5 which was used for the next step directly without further purification.

Step 6: Compound 119-6 was prepared from compound 119-5 following the procedure for the synthesis of compound 84-3 in example 25.

Step 7: 119-6 (160 mg) was purified by SFC (column: ChiralCel OX, 250×30 mm I.D., 10 μm, methanol/CO₂=30/70) to afford 119 (97.92 mg) and 120 (12.41 mg) respectively. 119: SFC analysis: 99.54% ee; retention time: 7.29 min; column: ChiralCel OX, 100×4.6 mm I.D., 3 μm, 40% of methanol (0.05% DEA) in C₀₂; pressure: 100 bar; flow rate: 2.5 mL/min. LCMS (ESI, m/z): [M+H]⁺=487.4. ¹H NMR (400 MHz, DMSO-d6, ppm): δ 9.49 (s, 1H), 8.93-8.91 (d, J=8.2 Hz, 1H), 8.58-8.56 (d, J=8.4 Hz, 1H), 5.51-5.21 (m, 1H), 5.12-4.91 (m, 1H), 4.03 (m, 1H), 3.50-3.47 (m, 1H), 3.04-3.02 (m, 1H), 2.68 (m, 2H), 2.16 (s, 1H), 1.96-1.72 (m, 4H), 1.44-1.03 (m, 4H), 0.97-0.76 (m, 11H); 120: SFC analysis: 98.66% ee; retention time: 8.28 min; column: ChiralCel OX, 100×4.6 mm I.D., 3 m, 40% of methanol (0.05% DEA) in CO₂; pressure: 100 bar; flow rate: 2.5 mL/min. LCMS (ESI, m/z): [M+H]⁺=487.4. ¹H NMR (400 MHz, DMSO-d6, ppm): δ 9.50 (s, 1H), 8.92-8.90 (d, J=8.7 Hz, 1H), 8.6-8.54 (d, J=8.6 Hz, 1H), 7.38-7.18 (m, 1H), 5.25-4.90 (m, 1H), 4.04-4.00 (m, 1H), 3.53-3.44 (m, 1H), 3.03-2.94 (m, 1H), 2.87-2.78 (m, 1H), 2.62-2.55 (m, 1H), 2.16-2.09 (m, 1H), 2.08-1.94 (m, 1H), 1.89-1.72 (m, 3H), 1.56-1.44 (m, 1H), 1.10-1.02 (m, 3H), 0.92-0.79 (m, 11H).

### Example 36 Synthesis of Compound 121 and 122

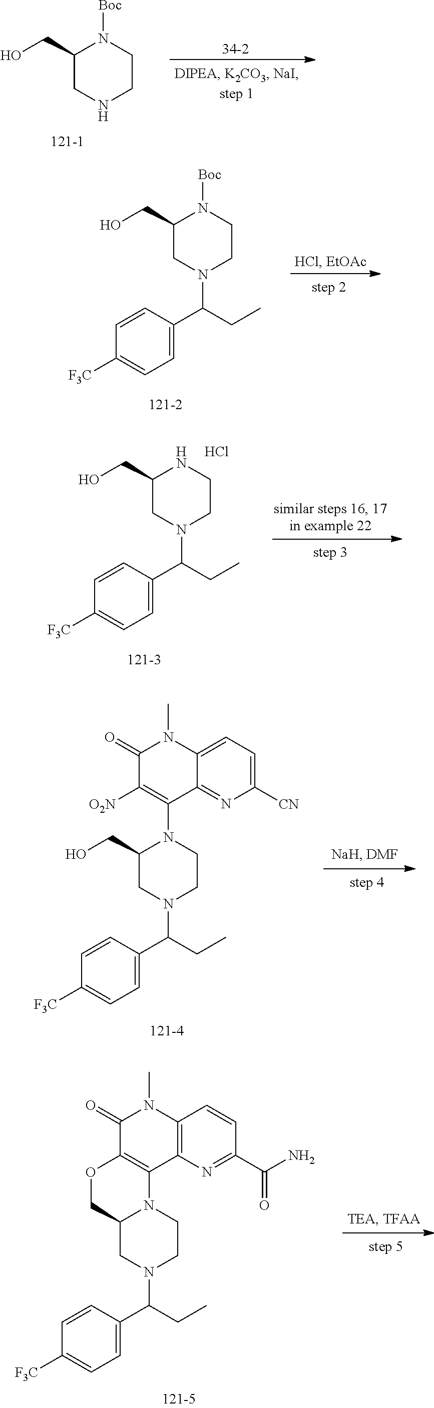

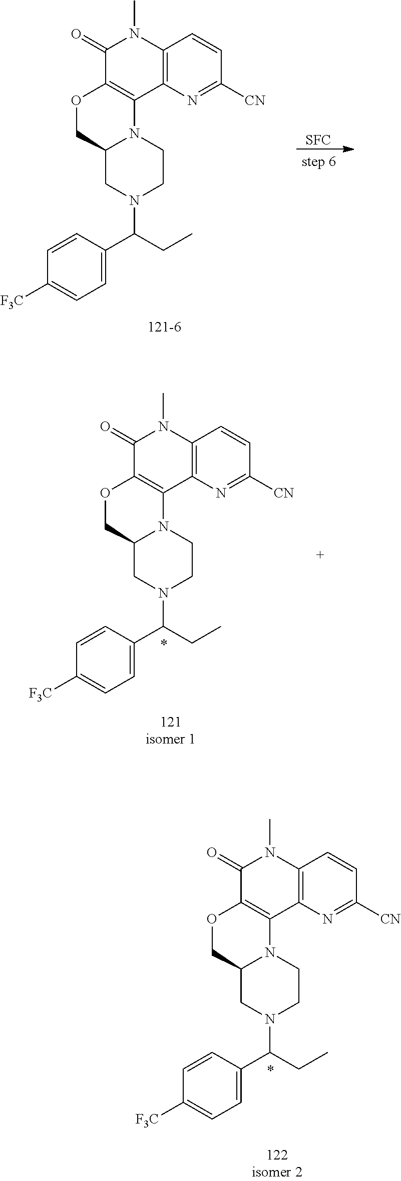 [SMILES: CCC(c1ccc(C(F)(F)F)cc1)N1CCN2c3c(c(=O)n(C)c4ccc(C#N)nc34)OC[C@@H]2C1 ; "SFC" ; CCC(c1ccc(C(F)(F)F)cc1)N1CCN2c3c(c(=O)n(C)c4ccc(C#N)nc34)OC[C@@H]2C1 ; CCC(c1ccc(C(F)(F)F)cc1)N1CCN2c3c(c(=O)n(C)c4ccc(C#N)nc34)OC[C@@H]2C1]

Step 1: A solution of 121-1 (4 g, 18.49 mmol), 34-2 (5.35 g, 24.04 mmol), NaI (0.6 g, 3.69 mmol) and DIPEA (7.64 mL, 46.24 mmol) in MeCN (30 mL) was stirred at 85° C. for 12 h. The mixture was concentrated. The residue was purified by column chromatography on silica gel (petroleum ether/ethyl acetate=1/0 to 1/1) to afford 121-2.

Step 2: To a solution of 121-2 (2 g, 4.97 mmol) in DCM (12 mL) was added HCl/ethyl acetate (6 mL) at 15° C. The mixture was stirred at 15° C. for 2 h. The mixture was concentrated to afford 121-3.

Step 3: Compound 121-4 was prepared from compound 121-3 following the procedure for the synthesis of compound 67-20 in example 22.

Step 4: To a solution of 121-4 (225 mg, 0.42 mmol) in DMF (30 mL) was added NaH (50.9 mg, 2.12 mmol) at 15° C. The mixture was stirred at 110° C. for 16 h. water was added to quench the reaction. The mixture was extracted with ethyl acetate. The organic phase was concentrated. The residue was purified by column chromatography on silica gel (DCM/MeOH=1/0 to 10/1) to afford 121-5.

Step 5: To a solution of 121-5 (130 mg, 0.26 mmol) in THF (6 mL) was added triethyl amine (0.072 mL, 0.52 mmol) and TFAA (0.14 mL, 1.04 mmol) at 0° C. The mixture was stirred at 15° C. for 48 h. The mixture was diluted with H₂O, extracted with ethyl acetate. The organic phase was concentrated. The residue was purified by reverse phase HPLC (0.5% TFA in water/MeCN, 5-70%) to afford 121-6.

Step 7: 121-6 (70 mg) was purified by SFC (column: ChiralCel OD, 250×30 mm I.D., 5 μm, Ethanol/CO₂=30/70) to afford 121 (18 mg) and 122 (5 mg) respectively. 121: SFC analysis: 100% ee; retention time: 2.69 min; column: ChiralCel OD, 150×4.6 mm I.D., 3 μm, 40% of ethanol (0.05% DEA) in C₀₂; pressure: 100 bar; flow rate: 2.5 mL/min. LCMS (ESI, m/z): [M+H]⁺=484.4. ¹H NMR (400 MHz, DMSO-d6, ppm): δ 8.13-8.00 (m, 2H), 7.78-7.66 (m, 2H), 7.58-7.42 (m, 2H), 4.80-4.50 (m, 1H), 4.34-4.20 (m, 1H), 4.12-3.96 (m, 1H), 3.58 (s, 3H), 3.50-3.42 (m, 1H), 3.34-3.23 (m, 2H), 2.84-2.70 (m, 2H), 2.35-2.25 (m, 1H), 2.22-2.10 (m, 1H), 2.03-1.88 (m, 1H), 1.83-1.68 (m, 1H), 0.73 (t, J=7.0 Hz, 3H). ¹⁹F NMR (376 MHz, DMSO-d6, ppm) δ −60.73 (3F); 122: SFC analysis: 99.34% ee; retention time: 3.16 min; column: ChiralCel OD, 150×4.6 mm I.D., 3 μm, 40% of ethanol (0.05% DEA) in C₀₂; pressure: 100 bar; flow rate: 2.5 mL/min. LCMS (ESI, m/z): [M+H]⁺=484.4. ¹H NMR (400 MHz, DMSO-d6, ppm): δ 8.11-7.97 (m, 2H), 7.78-7.66 (m, 2H), 7.56-7.42 (m, 2H), 4.65-4.35 (m, 1H), 4.32-4.20 (m, 1H), 4.16-4.02 (m, 1H), 3.58 (s, 3H), 3.51-3.40 (m, 1H), 3.33-3.25 (m, 2H), 2.92-2.76 (m, 1H), 2.70-2.57 (m, 1H), 2.34-2.20 (m, 2H), 2.02-1.89 (m, 1H), 1.80-1.66 (m, 1H), 0.74 (t, J=7.2 Hz, 3H). ¹⁹F NMR (376 MHz, DMSO-d6, ppm) δ −60.75 (3F).

### Example 37 Synthesis of Compound 123 and 124

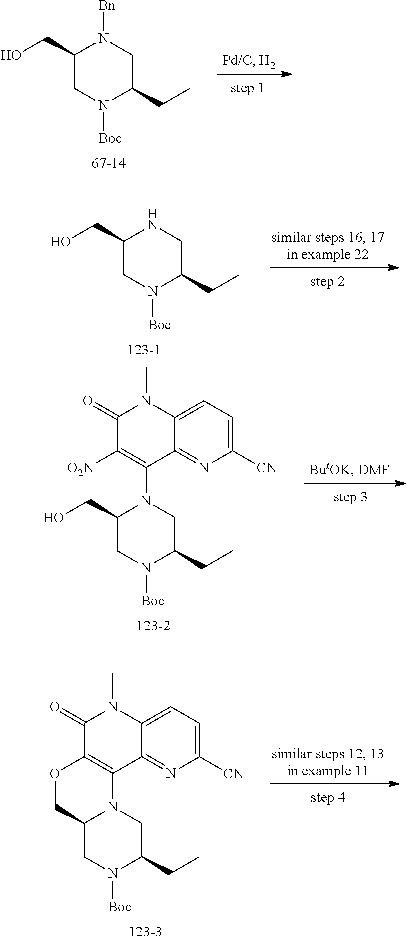

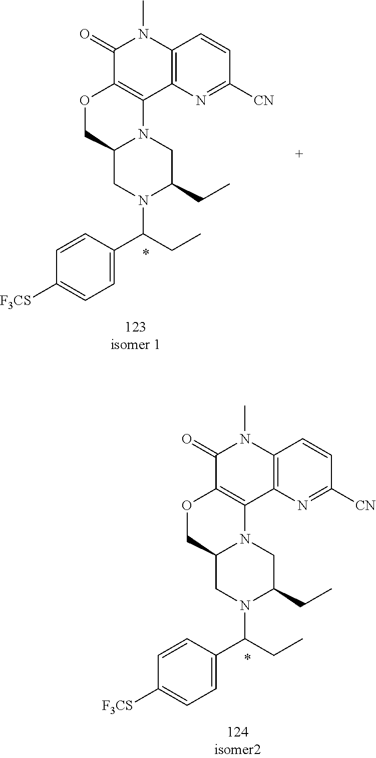

Step 1: To a stirred solution of 67-14 (2 g, 5.98 mmol) in isopropanol (35 mL) was added Pd/C (10%, 0.5 g, 0.470 mmol) at 25° C., the resulting mixture was stirred at 50° C. under H₂ balloon for 15 h. The reaction mixture was filtered and the filtrate was concentrated to afford 123-1.

Step 2: Compound 123-2 was prepared from compound 123-1 following the procedure for the synthesis of compound 67-20 in example 22.

Step 3: To a stirred solution of 123-2 (140 mg, 0.3 mmol) in DMF (15 mL) was added potassium tert-butoxide (99.7 mg, 0.89 mmol) at 15° C., the resulting mixture was stirred at 110° C. for 20 h. The reaction mixture was concentrated, the residue was purified by column chromatography on silica gel (petroleum ether/ethyl acetate=1/0 to 0/1) to afford 123-3.

Step 4: Compound 123 and 124 was prepared from compound 123-3 following the procedure for the synthesis of compound 28-15 in example 11. The product was obtained by column chromatography on silica gel (petroleum ether/ethyl acetate=1/0 to 0/1). 123: LCMS (ESI, m/z): [M+H]⁺=544.4. ¹H NMR (400 MHz, DMSO-d6, ppm): δ 8.11-8.06 (m, 2H), 7.71-7.69 (d, J=7.8 Hz, 2H), 7.53-7.51 (d, J=7.8 Hz, 2H), 5.32-5.25 (m, 1H), 4.16-4.14 (m, 1H), 3.83-3.79 (m, 1H), 3.61-3.52 (m, 4H), 3.35-3.31 (m, 1H), 3.09-2.99 (m, 2H), 2.38-2.33 (m, 2H), 2.01-1.86 (m, 2H), 1.79-1.72 (m, 2H), 1.04-1.00 (m, 3H), 0.59-0.52 (m, 3H). ¹⁹F NMR (376 MHz, DMSO-d6, ppm) δ −42.17 (3F). 124: LCMS (ESI, m/z): [M+H]⁺=544.4. ¹H NMR (400 MHz, DMSO-d6, ppm): δ 8.05 (s, 2H), 7.72-7.70 (d, J=8.0 Hz, 2H), 7.50-7.48 (d, J=7.8 Hz, 2H), 4.72-4.68 (m, 1H), 4.38-4.35 (m, 1H), 4.04-3.99 (m, 1H), 3.75-3.71 (m, 1H), 3.59 (s, 3H), 3.42-3.39 (m, 1H), 3.11-3.08 (m, 1H), 2.92-2.89 (m, 1H), 2.67-2.59 (m, 1H), 2.36-2.34 (m, 1H), 2.00-1.94 (m, 1H), 1.68-1.66 (m, 3H), 0.75-0.61 (m, 6H). ¹⁹F NMR (376 MHz, DMSO-d6, ppm) δ −42.31 (3F).

Compounds of the present disclosure can be synthesized by those having ordinary skill in the art in view of the present disclosure. Representative further compounds synthesized by following similar procedures/methods described herein in the Examples section. The structures and representative analytical data are shown in Table 1 below.

TABLE 1
Characterization of exemplary compounds of the present disclosure

| Com- | | | |
|---|---|---|---|
| pound | | | |
| No. | Structure | [M + H]⁺ | ¹H-NMR |
| 6 | 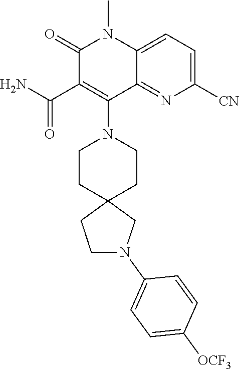 [SMILES: Cn1c(=O)c(C(N)=O)c(N2CCC3(CCN(c4ccc(OC(F)(F)F)cc4)C3)CC2)c2nc(C#N)ccc21] | 527.2 | 0.33 TFA salt, ¹H NMR (400 MHz, DMSO-d6, ppm) δ 8.21-8.18 (m, 1H), 8.11-8.09 (m, 1H), 7.68 (s, 1H), 7.52 (s, 1H), 7.16-7.14 (m, 2H), 6.59-6.57 (m, 2H), 3.57 (s, 7H), 3.39-3.34 (m, 2H), 3.21 (s, 2H), 1.96 (t, J = 6.9 Hz, 2H), 1.78 (s, 4H). ¹⁹F NMR (376 MHz, DMSO-d6, ppm) δ −57.36 (3F), −74.30 (1F, TFA). |
| 7 | 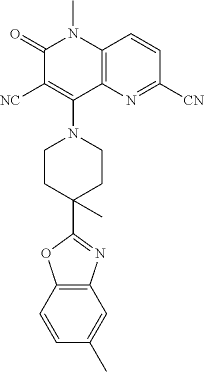 [SMILES: Cc1ccc2oc(C3(C)CCN(c4c(C#N)c(=O)n(C)c5ccc(C#N)nc45)CC3)nc2c1] | 439.4 | ¹H NMR (400 MHz, DMSO-d6, ppm) δ 8.29-8.27 (m, 1H), 8.17- 8.15 (m, 1H), 7.63-7.55 (m, 2H), 7.23-7.16 (m, 1H), 4.15-4.05 (m, 2H), 3.73-3.62 (m, 2H), 3.53 (s, 3H), 2.55-2.52 (m, 1H), 2.50- 2.47 (m, 1H), 2.42 (s, 3H), 2.07-1.98 (m, 2H), 1.50 (s, 3H). |
| 8 | 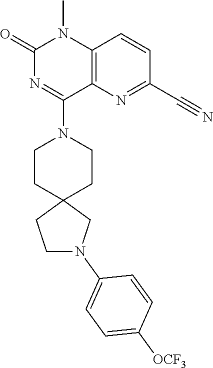 [SMILES: Cn1c(=O)nc(N2CCC3(CCN(c4ccc(OC(F)(F)F)cc4)C3)CC2)c2nc(C#N)ccc21] | 485.4 | 0.45 TFA salt, ¹H NMR (400 MHz, DMSO-d6, ppm) δ 8.25 (d, J = 8.8 Hz, 1H), 8.00 (d, J = 8.8 Hz, 1H), 7.14 (d, J = 8.4 Hz, 2H), 6.56 (d, J = 8.0 Hz, 2H), 3.58-3.48 (m, 4H) 3.44 (s, 3H), 3.34 (t, J = 6.8 Hz, 2H), 3.22 (s, 2H), 1.96 (t, J = 6.8 Hz, 2H), 1.77-1.65 (m, 4H). ¹⁹F NMR (376 MHz, DMSO-d6, ppm) δ −57.37 (3F), −74.44 (1.36F, TFA). |
| 9 | 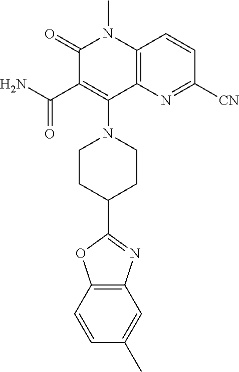 [SMILES: Cc1ccc2oc(C3CCN(c4c(C(N)=O)c(=O)n(C)c5ccc(C#N)nc45)CC3)nc2c1] | 443.4 | ¹H NMR (400 MHz, DMSO-d6, ppm) δ 8.20 (d, J = 8.8 Hz, 1H), 8.11 (d, J = 8.8 Hz, 1H), 7.70 (s, 1H), 7.59-7.53 (m, 3H), 7.20-7.17 (dd, J = 8.0, 1.2 Hz, 1H), 3.82-3.79 (m, 2H), 3.57 (s, 3H), 3.38-3.25 (m, 3H), 2.42 (s, 3H), 2.22-2.19 (m, 2H), 2.15-2.06 (m, 2H). |
| 10 | 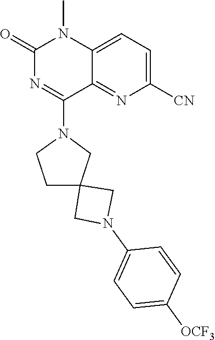 [SMILES: Cn1c(=O)nc(N2CCC3(CN(c4ccc(OC(F)(F)F)cc4)C3)C2)c2nc(C#N)ccc21] | 457.4 | ¹H NMR (400 MHz, DMSO-d₆, ppm) δ 8.26-8.24 (m, 1H), 7.99-7.96 (m, 1H), 7.18-7.15 (m, 2H), 6.51-6.49 (m, 2H), 4.44 (s, 1H), 4.30 (t, J = 6.8 Hz, 1H, 3.90-3.73 (m, 6H), 3.45 (s, 3H), 2.32 (t, J = 6.8 Hz, 1H), 2.17 (t, J = 6.8 Hz, 1H). ¹⁹F NMR (376 MHz, DMSO-d6, ppm): δ −57.32 (3F). |
| 11 | 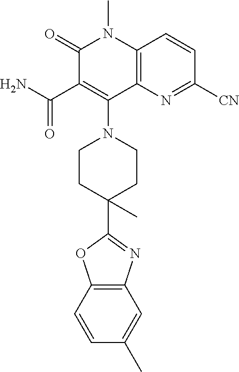 [SMILES: Cc1ccc2oc(C3(C)CCN(c4c(C(N)=O)c(=O)n(C)c5ccc(C#N)nc45)CC3)nc2c1] | 457.4 | ¹H NMR (400 MHz, DMSO-d₆, ppm) δ 8.20-8.19 (m, 1H), 8.09- 8.10 (m, 1H), 7.64-7.45 (m, 4H), 7.18-7.20 (m, 1H), 3.63-3.66 (m, 2H). 3.56 (s, 1H), 3.19-3.24 (m, 1H), 2.40-2.49 (m, 5H), 1.95- 2.00 (m, 2H), 1.45 (s, 3H). |
| 13 | 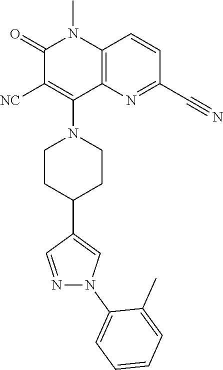 [SMILES: Cc1ccccc1-n1cc(C2CCN(c3c(C#N)c(=O)n(C)c4ccc(C#N)nc34)CC2)cn1] | 450.4 | ¹H NMR (400 MHz, CDCl₃-d, ppm) δ 7.89-7.87 (m, 1H), 7.76- 7.74 (m, 1H), 7.66 (s, 1H), 7.49 (s, 1H), 7.33-7.30 (m, 4H), 4.47-4.44 (m, 2H), 3.72-3.62 (m, 5H), 3.07-3.01 (m, 1H), 2.28- 2.25 (m, 5H), 2.10-2.00 (m, 2H). |
| 14 | 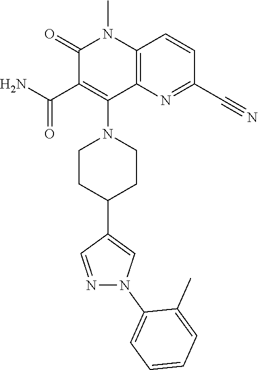 [SMILES: Cc1ccccc1-n1cc(C2CCN(c3c(C(N)=O)c(=O)n(C)c4ccc(C#N)nc34)CC2)cn1] | 468.4 | ¹H NMR (400 MHz, CDCl₃-d, ppm) δ 8.14 (s, 1H), 7.83-7.80 (m, 1H), 7.72-7.70 (m, 1H), 7.63 (s, 1H), 7.44 (s, 1H), 7.30-7.27 (m, 4H), 6.15 (s, 1H), 4.03-4.00 (m, 2H), 3.64 (s, 3H), 3.44-3.38 (m, 2H), 3.04-2.99 (m, 1H), 2.24 (s, 3H), 2.14-2.12 (m, 2H), 2.03-2.00 (m, 2H). |
| 15 | 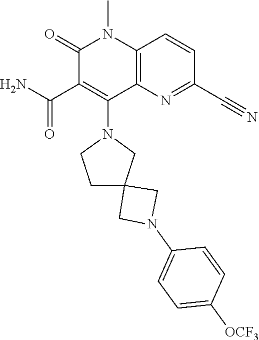 [SMILES: Cn1c(=O)c(C(N)=O)c(N2CCC3(CN(c4ccc(OC(F)(F)F)cc4)C3)C2)c2nc(C#N)ccc21] | 499.4 | 0.48 TFA salt, ¹H NMR (400 MHz, DMSO-d6, ppm) δ 8.18 (d, J = 8.8 Hz, 1H), 8.04 (d, J = 8.9 Hz, 1H), 7.86 (s, 1H), 7.35 (s, 1H), 7.17-7.15 (m, 2H), 6.49-6.51 (m, 2H), 4.11 (s, 2H), 3.97-4.00 (m, 2H), 3.81 (s, 4H), 3.53 (s, 4H), 2.15-2.18 (m, 2H). ¹⁹F NMR (376 MHz, DMSO-d6, ppm) δ −57.33 (3F), −74.28 (1.43F, TFA). |
| 18 | 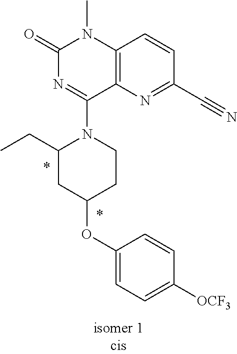 [SMILES: CCC1CC(Oc2ccc(OC(F)(F)F)cc2)CCN1c1nc(=O)n(C)c2ccc(C#N)nc12] | 474.2 | SFC analysis: 99.04% ee; retention time: 2.76 min; column: DAICELCHIRALPAK®WHELK, methanol (0.1% DEA) in CO2, 40%; pressure: 1800 psi: flow rate: 3.0 mL/min. ¹H NMR (400 MHz, DMSO-d6, ppm): δ 8.26-8.19 (m, 1H), 8.02-7.94 (m, 1H), 7.32-7.19 (m, 2H), 7.11-7.01 (m, 2H), 4.96-4.78 (m, 1H), 3.42 (s, 3H), 3.34-3.30 (m, 3H), 2.22-2.09 (m, 2H), 2.02-1.82 (m, 2H), 1.75-1.62 (m, 1H), 1.60-1.44 (m, 1H), 1.00-0.80 (m, 3H). ¹⁹F NMR (376 MHz, DMSO-d6, ppm): δ −57.25 (3F). |
| 19 | 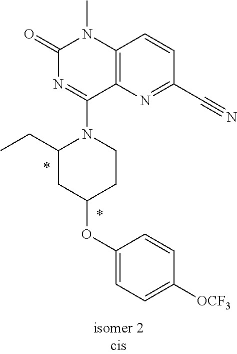 [SMILES: CCC1CC(Oc2ccc(OC(F)(F)F)cc2)CCN1c1nc(=O)n(C)c2ccc(C#N)nc12] | 474.2 | SFC analysis: 99.4% ee; retention time: 4.47 min; column: DAICELCHIRALPAK®WHELK, methanol (0.1% DEA) in CO2, 40%; pressure: 1800 psi: flow rate: 3.0 mL/min. ¹H NMR (400 MHz, DMSO-d6, ppm): δ 8.29-8.22 (m, 1H), 8.05-7.98 (m, 1H), 7.36-7.25 (m, 2H), 7.14-7.03 (m, 2H), 4.95-4.82 (m, 1H), 3.46 (s, 3H), 3.38-3.34 (m, 3H), 2.30-2.10 (m, 2H), 2.02-1.88 (m, 2H), 1.78-1.66 (m, 1H), 1.62-1.47 (m, 1H), 1.02-0.82 (m, 3H). ¹⁹F NMR (376 MHz, DMSO-d6, ppm): δ −57.25 (3F). |
| 20 | 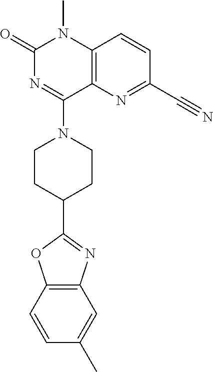 [SMILES: Cc1ccc2oc(C3CCN(c4nc(=O)n(C)c5ccc(C#N)nc45)CC3)nc2c1] | 401.4 | ¹H NMR (400 MHz, CDCl₃-d, ppm) δ 7.80-7.77 (m, 1H), 7.56-7.50 (m, 1H), 7.40 (s, 1H), 7.33-7.26 (m, 1H), 7.07-7.05 (m, 1H), 5.60- 5.50 (m, 1H), 5.10-4.90 (m, 1H), 3.90-3.70 (m, 1H), 3.52 (s, 3H), 3.50-3.25 (m, 2H) 2.39 (s, 3H), 2.35-2.25 (m, 2H), 2.20-2.05 (m, 2H). |
| 22 | 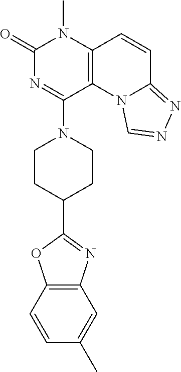 [SMILES: Cc1ccc2oc(C3CCN(c4nc(=O)n(C)c5ccc6nncn6c45)CC3)nc2c1] | 416.2 | ¹H NMR (400 MHz, DMSO-d6) δ 9.52-9.43 (m, 1H), 8.28-8.23 (m, 1H), 7.80-7.75 (m, 1H), 7.62-7.43 (m, 2H), 7.26-7.09 (m, 1H), 3.79-3.71 (m, 3H), 3.63-3.57 (m, 6H), 3.14-3.04 (m, 1H), 2.45-2.38 (m, 3H), 2.29-2.23 (m, 2H), 2.13-2.05 (m, 1H), 1.95- 1.84 (m, 1H) |
| 31 | 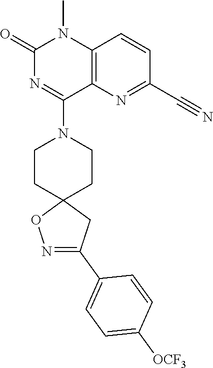 [SMILES: Cn1c(=O)nc(N2CCC3(CC2)CC(c2ccc(OC(F)(F)F)cc2)=NO3)c2nc(C#N)ccc21] | 485.2 | ¹H NMR (400 MHz, DMSO-d6): δ 8.30-8.24 (m, 1H), 8.06- 8.00 (m, 1H), 7.86-7.72 (m, 2H), 7.55-7.40 (m, 2H), 4.80-3.85 (m, 4H), 3.47 (s, 3H), 3.37-3.34 (m, 2H), 2.05-1.89 (m, 4H). ¹⁹F NMR (376 MHz, DMSO-d6, ppm): δ −56.74 (3F). |
| 38 | 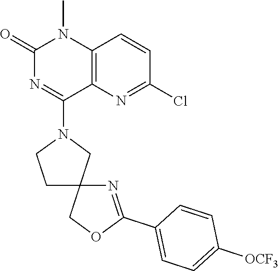 [SMILES: Cn1c(=O)nc(N2CCC3(COC(c4ccc(OC(F)(F)F)cc4)=N3)C2)c2nc(Cl)ccc21] | 480.2 | ¹H NMR (400 MHz, DMSO-d6): δ 8.32-8.20 (m, 1H), 8.07- 7.92 (m, 3H), 7.53-7.40 (m, 2H), 4.65-4.28 (m, 4H), 3.96-3.78 (m, 2H), 3.50-3.39 (m, 3H), 2.33-2.20 (m, 1H), 2.19-2.09 (m, 1H). ¹⁹F NMR (376 MHz, DMSO-d6, ppm): δ −56.68 (3F). |
| 40 | 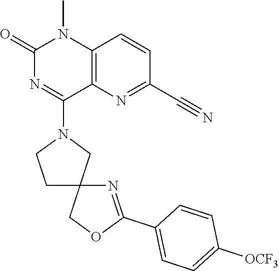 [SMILES: Cn1c(=O)nc(N2CCC3(COC(c4ccc(OC(F)(F)F)cc4)=N3)C2)c2nc(C#N)ccc21] | 471.2 | ¹H NMR (400 MHz, DMSO-d6): δ 8.06-7.96 (m, 2H), 7.95- 7.86 (m, 1H), 7.84-7.74 (m, 1H), 7.47 (d, J = 8.2 Hz, 2H), 4.65- 4.21 (m, 4H), 3.95-3.73 (m, 2H), 3.49-3.39 (m, 3H), 2.30-2.07 (m, 2H). ¹⁹F NMR (376 MHz, DMSO-d6, ppm): δ −56.68 (3F). |
| 41 | 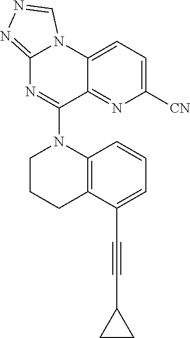 [SMILES: N#Cc1ccc2c(n1)c(N1CCCc3c(C#CC4CC4)cccc31)nc1nncn12] | 392.2 | ¹H NMR (400 MHz, DMSO-d6, ppm): δ 9.70 (s, 1H), 8.97-8.95 (m, 1H), 8.52-8.50 (m, 1H), 7.12-7.10 (m, 1H), 6.98-6.92 (m, 2H), 4.08-4.05 (m, 2H), 2.89-2.86 (m, 2H), 2.03-2.00 (m, 2H), 1.62- 1.59 (m, 1H), 0.94-0.92 (m, 2H), 0.80-0.78 (m, 2H). |
| 43 | 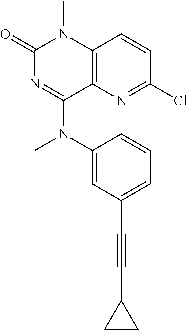 [SMILES: CN(c1cccc(C#CC2CC2)c1)c1nc(=O)n(C)c2ccc(Cl)nc12] | 365.2 | LCMS (ESI, m/z): ¹H NMR (400 MHz, DMSO-d6, ppm) δ 7.88 (d, J = 8.0 Hz, 1H), 7.63 (d, J = 8.8 Hz, 1H), 7.32-7.10 (m, 4H), 3.49- 3.46 (m, 6H), 1.56-1.48 (m, 1H), 0.89-0.85 (m, 2H), 0.73-0.70 (m, 2H). |
| 46 | 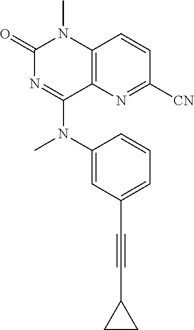 [SMILES: CN(c1cccc(C#CC2CC2)c1)c1nc(=O)n(C)c2ccc(C#N)nc12] | 356.2 | 0.17 TFA salt, ¹H NMR (400 MHz, DMSO-d6, ppm) δ 8.15 (s, 0.17H), 8.07 (d, J = 12.0 Hz, 1H), 7.96 (d, J = 12.0 Hz, 1H), 7.34- 7.20 (m, 4H), 3.50-3.48 (m, 6H), 1.54-1.49 (m, 1H), 0.88-0.84 (m, 2H), 0.74-0.71 (m, 2H). |
| 47 | 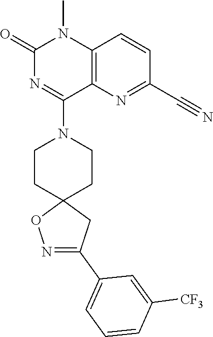 [SMILES: Cn1c(=O)nc(N2CCC3(CC2)CC(c2cccc(C(F)(F)F)c2)=NO3)c2nc(C#N)ccc21] | 469.2 | ¹H NMR (400 MHz, DMSO): δ 8.20 (d, J = 8.9 Hz, 1H), 7.97-7.93 (m, 2H), 7.90 (s, 1H), 7.77 (d, J = 7.9 Hz, 1H), 7.69-7.64 (m, 1H), 4.70-3.90 (m, 4H), 3.42 (s, 3H), 3.40 (s, 2H), 2.01-1.89 (m, 4H). ¹⁹F NMR (376 MHz, DMSO-d6, ppm): δ −61.43 (3F). |
| 48 | 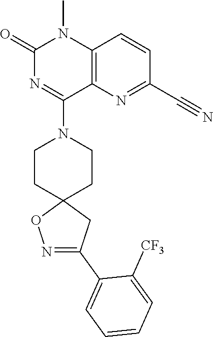 [SMILES: Cn1c(=O)nc(N2CCC3(CC2)CC(c2ccccc2C(F)(F)F)=NO3)c2nc(C#N)ccc21] | 469.2 | ¹H NMR (400 MHz, DMSO): δ 8.22 (d, J = 8.9 Hz, 1H), 7.97 (d, J = 9.0 Hz, 1H), 7.84 (d, J = 7.8 Hz, 1H), 7.78-7.74 (m, 1H), 7.69-7.64 (m, 2H), 4.65-3.95 (m, 4H), 3.43 (s, 3H), 3.28 (s, 2H), 2.02-1.94 (m, 4H). ¹⁹F NMR (376 MHz, DMSO-d6, ppm): δ −57.42 (3F). |
| 49 | 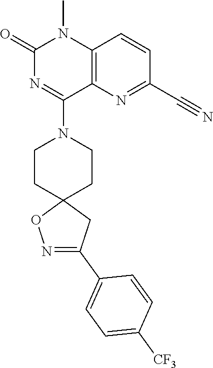 [SMILES: Cn1c(=O)nc(N2CCC3(CC2)CC(c2ccc(C(F)(F)F)cc2)=NO3)c2nc(C#N)ccc21] | 469.2 | ¹H NMR (400 MHz, DMSO-d6, ppm): δ 8.31-8.23 (m, 1H), 8.07- 7.99 (m, 1H), 7.93-7.79 (m, 4H), 4.95-3.75 (m, 4H), 3.46 (s, 3H), 3.39 (s, 2H), 2.05-1.89 (m, 4H). ¹⁹F NMR (376 MHz, DMSO-d6, ppm): δ −61.23 (3F). |
| 51 | 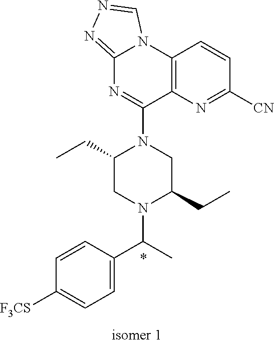 | 541.4 | Analytical HPLC*: retention time: 1.45 min; 1.0 TFA salt, ¹H NMR (400 MHz, DMSO-d6, ppm) δ 9.50 (s, 1H), 8.92-8.90 (m, 1H), 8.56- 8.54 (m, 1H), 8.40 (s, 1H), 7.73-7.68 (m, 2H), 7.58-7.54 (m, 2H), 5.34-5.01 (m, 2H), 3.86-3.81 (m, 1H), 2.99-2.91 (m, 1H), 2.80-2.77 (m, 1H), 2.36 (s, 1H), 2.16 (s, 1H), 1.97-1.90 (m, 2H), 1.51-1.45 (m, 2H), 1.32-1.30 (m, 3H), 1.00-0.94 (m, 3H), 0.75- 0.68 (m, 3H). ¹⁹F NMR (376 MHz, DMSO-d₆, ppm) δ −42.24 (3F). |
| 52 | 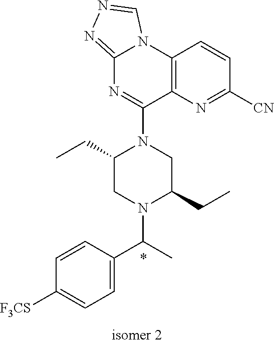 | 541.4 | Analytical HPLC*: retention time: 1.50 min; 1.0 TFA salt, ¹H NMR (400 MHz, DMSO-d6, ppm) δ 9.49 (s, 1H), 8.92-8.90 (m, 1H), 8.55- 8.53 (m, 1H), 8.40 (s, 1H), 7.71-7.69 (m, 2H), 7.59-7.56 (m, 2H), 5.22-4.90 (m, 1H), 3.68-3.66 (m, 1H), 2.66-2.59 (m, 1H), 2.23-2.20 (m, 1H), 2.07-1.99 (m, 2H), 1.74-1.80 (m, 1H), 1.62 (s, 1H), 1.47-1.45 (m, 2H), 1.33-1.24 (m, 4H), 1.00-1.00 (m, 3H), 0.61-0.58 (m, 3H). ¹⁹F NMR (376 MHz, DMSO-d₆, ppm) δ −42.38 (3F). |
| 53 | 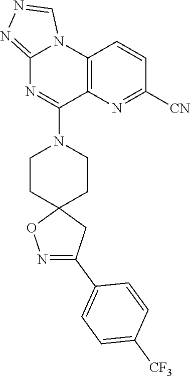 [SMILES: N#Cc1ccc2c(n1)c(N1CCC3(CC1)CC(c1ccc(C(F)(F)F)cc1)=NO3)nc1nncn12] | 479.2 | ¹H NMR (400 MHz, DMSO-d6, ppm): δ 9.57 (s, 1H), 9.00-8.91 (m, 1H), 8.63-8.54 (m, 1H), 7.94-7.80 (m, 4H), 4.45-4.26 (m, 2H), 4.25-4.04 (m, 2H), 3.41 (s, 2H), 2.12-1.94 (m, 4H). ¹⁹F NMR (376 MHz, DMSO-d6, ppm): δ −61.22 (3F). |
| 54 | 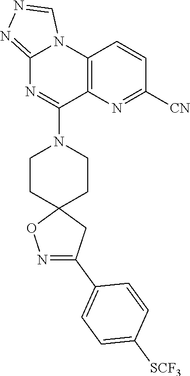 | 511.2 | ¹H NMR (400 MHz, DMSO-d6, ppm): δ 9.56 (s, 1H), 8.98-8.92 (m, 1H), 8.61-8.55 (m, 1H), 7.88-7.75 (m, 4H), 4.45-4.26 (m, 2H), 4.25-4.08 (m, 2H), 3.39 (s, 2H), 2.11-1.92 (m, 4H).¹⁹F NMR (376 MHz, DMSO-d6, ppm): δ −41.86 (3F). |
| 55 | 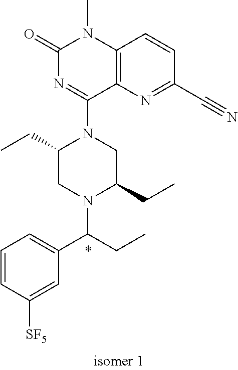 | 571.5 | Analytical HPLC*: retention time: 1.75 min; 2.0 TFA salt, ¹H NMR (400 MHz, DMSO-d6, ppm) δ 8.43-7.44 (m, 6H), 6.27-5.67 (m, 1H), 5.39-4.92 (m, 1H), 4.91-4.56 (m, 1H), 4.07-3.60 (m, 1H), 3.47 (s, 3H), 3.42-3.24 (m, 1H), 3.21-2.63 (m, 1H), 2.51-2.43 (m, 2H), 2.42- 1.89 (m, 3H), 1.88-1.77 (m, 1H), 1.76-1.66 (m, 1H), 1.12-0.93 (m, 3H), 0.92-0.26 (m, 6H); ¹⁹F NMR (376 MHz, DMSO-d6, ppm): δ 86.51 (1F), 63.98 (4F), −74.47 (6F, TFA). |
| 56 | 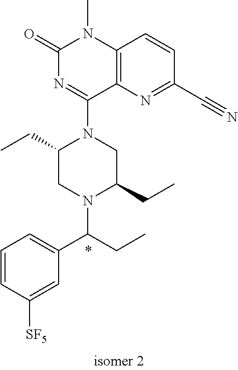 | 571.5 | Analytical HPLC*: retention time: 1.79 min; 2.0 TFA salt, ¹H NMR (400 MHz, DMSO-d6, ppm) δ 8.42-7.41 (m, 6H), 6.51-5.21 (m, 1H), 5.19-4.50 (m, 1H), 4.23-3.53 (m, 2H), 3.47 (s, 3H), 3.36-3.04 (m, 1H), 2.82-2.51 (m, 1H), 2.42-2.19 (m, 1H), 2.17-1.22 (m, 6H), 1.21- 0.76 (m, 3H), 0.75-0.38 (m, 6H); ¹⁹F NMR (376 MHz, DMSO-d6, ppm) δ 86.41 (1F), 64.84 (4F), −74.58 (6F, TFA). |
| 60 | 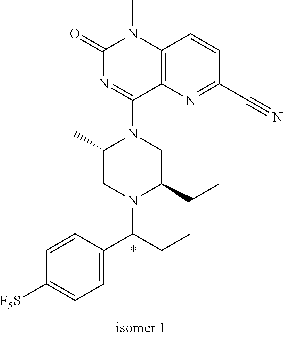 | 557.4 | SFC analysis; 100% de; retention time: 4.12 min; column: REGIS (S,S)WHELK-O1, MeOH (0.1% of DEA) in CO2, 5% to 40%; pressure: 100 bar; flow rate: 1.5 mL/min. ¹H NMR (400 MHz, DMSO-d₆, ppm) δ 8.24 (d, J = 8.0 Hz, 1H), 7.99 (d, J = 8.0 Hz, 1H), 7.91 (d, J = 8.0 Hz, 2H), 7.55 (d, J = 8.0 Hz, 2H), 5.90-5.54 (m, 1H), 5.01-4.72 (m, 1H) 3.70-3.62 (m, 1H), 3.43 (s, 3H), 3.20-3.11 (m, 1H), 2.94-2.83 (m, 1H), 2.71-2.53 (m, 1H), 2.39-2.30 (m, 1H), 1.97- 1.82 (m, 1H), 1.70-1.58 (m, 1H), 1.51-1.40 (m, 2H, 1.49-1.23 (m, 3H), 0.76-0.60 (m, 6H); ¹⁹F NMR (376 MHz, DMSO-d₆, ppm): δ 88.20 (1F), 64.42 (4F). |
| 61 | 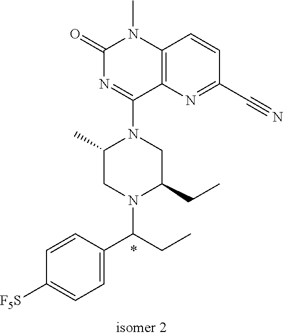 | 557.4 | SFC analysis; 81.04% de; retention time: 4.92 min; column: REGIS (S,S)WHELK-O1, MeOH (0.1% of DEA) in CO2, 5% to 40%; pressure: 100 bar; flow rate: 1.5 mL/min. ¹H NMR (400 MHz, DMSO-d₆, ppm) δ 8.28-8.20 (m, 1H), 8.01 (d, J = 8.0 Hz, 1H), 7.88 (d, J = 8.0 Hz, 2H), 7.59 (d, J = 8.0 Hz, 2H), 6.03-5.49 (m, 1H), 5.40-4.85 (m, 1H) 3.69-3.52 (m, 1H), 3.43 (s, 3H), 3.30-3.25 (m, 1H), 3.19-3.14 (m, 1H), 2.62-2.58 (m, 1H), 2.19-2.05 (m, 1H), 1.82- 1.79 (m, 1H), 1.61-1.50 (m, 1H), 1.47-1.36 (m, 2H), 1.32-1.27 (m, 3H),1.05-0.95 (m, 3H), 0.62-0.57 (m, 3H); ¹⁹F NMR (376 MHz, DMSO-d₆, ppm): δ 88.20 (1F), 64.42 (4F). |
| 62 | 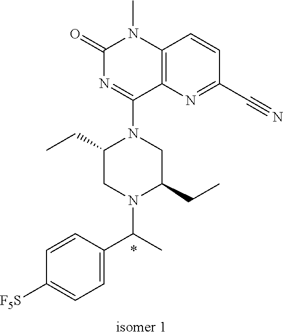 | 557.4 | Analytical HPLC*: retention time: 1.72 min; 2.57 TFA salt, ¹H NMR (400 MHz, CDCl₃-d, ppm): δ 7.93-7.69 (m, 6H), 6.36-6.01 (m, 1H), 5.54-5.42 (m, 1H), 4.26-4.13 (m, 1H), 3.62 (s, 3H), 3.36-3.31 (m, 1H), 3.19-3.08 (m, 2H), 2.18-2.01 (m, 2H), 1.91-1.68 (m, 6H), 0.91- 0.69 (m, 6H). ¹⁹F NMR (376 MHz, CDCl₃-d, ppm): δ 82.75 (1F), 62.61 (4F), −75.98 (7.7F, TFA). |
| 63 | 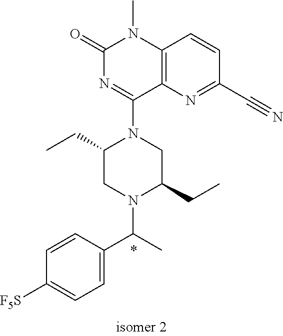 | 557.4 | Analytical HPLC*: retention time: 1.75 min; 2.0 TFA salt, ¹H NMR (400 MHz, CDCl₃-d, ppm): δ 7.90-7.72 (m, 6H), 6.53-6.14 (m, 1H), 5.56-5.34 (m, 1H), 4.14-4.05 (m, 2H), 3.63 (s, 3H), 3.31-3.24 (m, 2H), 2.99-2.98 (m, 1H), 2.28-2.21 (m, 2H), 2.11-1.83 (m, 5H), 1.25- 1.21 (m, 3H), 0.75-0.72 (m, 3H). ¹⁹F NMR (376 MHz, CDCl₃-d, ppm): δ 82.62 (1F), 62.61 (4F), −75.84 (6F, TFA). |
| 65 | 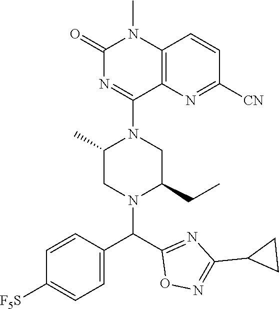 | 637.4 | ¹H NMR (400 MHz, Methanol-d4, ppm) δ 8.09-8.04 (m, 1H), 8.02- 7.95 (m, 1H), 7.93-7.84 (m, 2H), 7.80-7.76 (m, 2H), 6.33-5.78 (m, 1H), 5.33 (s, 1H), 5.28-5.03 (m, 1H), 3.78-3.64 (m, 1H), 3.57 (s, 3H), 3.10-2.79 (m, 1H), 2.71-2.52 (m, 1H), 2.44-2.30 (m, 1H), 2.20-2.06 (m, 1H), 1.73-1.52 (m, 3H), 1.15-1.05 (m, 2H), 1.05-0.96 (m, 2H), 0.95-0.87 (m, 3H), 0.86-0.78 (m, 2H). ¹⁹F NMR (376 MHz, Methanol-d4): δ 82.48 (1F), 61.33 (4F). |
| 66 | 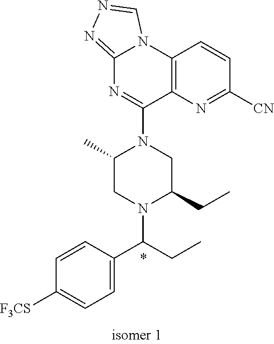 | 541.4 | Analytical HPLC*: retention time: 1.55 min; 2.7 TFA salt, ¹H NMR (400 MHz, DMSO-d6, ppm) δ 9.65-9.62 (m, 1H), 9.00 (d, J = 8.4 Hz, 1H), 8.68-8.62 (m, 1H), 7.93-7.53 (m, 4H), 5.60-5.05 (m, 1H), 4.62-4.49 (m, 1H), 4.01-3.90 (m, 1H), 3.68-3.57 (m, 1H), 3.47-3.40 (m, 1H), 3.02-2.98 (m, 1H), 2.45-2.34 (m, 1H), 2.02-1.90 (m, 1H), 1.77-1.53 (m, 3H), 1.44-1.25 (m, 3H), 0.90-0.43 (m, 6H). ¹⁹F NMR (376 MHz, DMSO-d6, ppm): δ −41.82 (3F), −73.93 (8.1 F, TFA). |
| 68 | 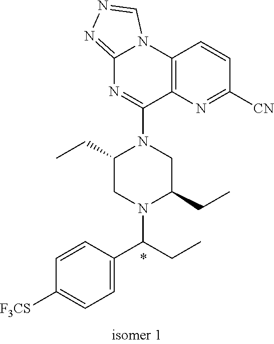 | 555.4 | Chiral HPLC analysis: 97.47% de; retention time: 4.84 min; column: Daicel CHIRALPAK®IG, EtOH (0.2% of DEA)/n-Hexane = 5/95, flow rate: 1.0 mL/min. ¹H NMR (400 MHz, DMSO-d6, ppm) δ 9.48 (s, 1H), 8.91-8.89 (m, 1H), 8.55-8.53 (m, 1H), 7.73-7.71 (m, 2H), 7.52-7.50 (m, 2H), 5.54-4.71 (m, 1H), 2.97-2.91 (m, 1H), 2.85-2.79 (m, 1H), 2.37-2.33 (m, 1H), 2.09 (s, 1H), 1.98-1.88 (m, 2H), 1.67-1.56 (m, 2H), 1.48-1.42 (m, 2H), 1.25 (s, 2H), 0.99- 0.94 (m, 3H), 0.65-0.62 (m, 6H). ¹⁹F NMR (376 MHz, DMSO-d6, ppm) δ −42.27 (s, 3F). |
| 69 | 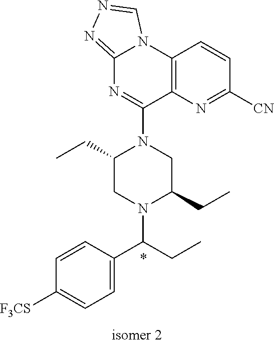 | 555.4 | Chiral HPLC analysis: 81.52% de; retention time: 5.87 min; column: Daicel CHIRALPAK®IG, EtOH (0.2% of DEA)/n-Hexane = 5/95, flow rate: 1.0 mL/min. ¹H NMR (400 MHz, DMSO-d6, ppm) δ 9.49 (s, 1H), 8.91-8.89 (m, 1H), 8.55-8.53 (m, 1H), 7.71-7.69 (m, 2H), 7.54-7.52 (m, 2H), 5.41-4.65 (m, 1H), 3.51-3.45 (m, 1H), 3.16 (s, 1H), 2.25-2.14 (m, 1H), 2.07-1.97 (m, 1H), 1.91-1.83 (m, 1H), 1.78-1.71 (m, 1H), 1.65-1.49 (m, 2H), 1.49-1.41 (m, 1H), 1.27-1.19 (m, 3H), 1.06-0.98 (m, 3H), 0.63-0.55 (m, 6H). ¹⁹F NMR (376 MHz, DMSO-d6, ppm) δ −42.44 (s, 3F). |
| 70 | 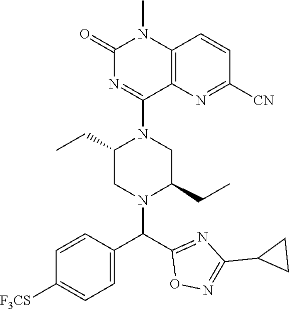 | 625.4 | ¹H NMR (400 MHz, DMSO-d6, ppm) δ 8.29-8.17 (m, 1H), 8.04- 7.94 (m, 1H), 7.84-7.70 (m, 4H), 6.03-5.27 (m, 2H), 5.03-4.75 (m, 1H), 3.68-3.48 (m, 1H), 3.44 (s, 3H), 3.29-2.80 (m, 1H), 2.71- 2.54 (m, 1H), 2.42-2.31 (m, 1H), 2.20-1.69 (m, 3H), 1.65-1.28 (m, 2H), 1.14-1.02 (m, 2H), 0.96-0.54 (m, 8H). ¹⁹F NMR (376 MHz, DMSO-d6, ppm) δ −41.97 (br. s, 3F). |
| 71 | 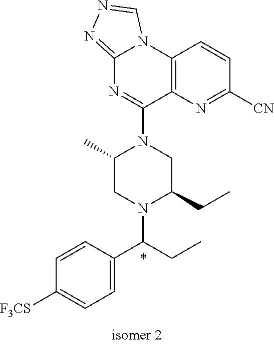 | 541.4 | Analytical HPLC*: retention time: 1.52 min; 0.34 FA/0.56 TFA salt, ¹H NMR (400 MHz, DMSO-d6, ppm): δ 9.51 (s, 1H), 8.91 (d, J = 8.6 Hz, 1H), 8.55 (d, J = 8.6 Hz, 1H), 8.14 (s, 0.34H), 7.70 (d, J = 8.0 Hz, 2H), 7.54 (d, J = 8.0 Hz, 2H), 5.62-5.41 (m, 1H), 5.02-4.82 (m, 1H), 3.61-3.52 (m, 2H), 3.20-3.10 (m, 1H), 2.70-2.62 (m, 1H), 2.16-2.13 (m, 1H), 1.89-1.86 (m, 1H), 1.78-1.72 (m, 1H), 1.67-1.63 (m, 1H), 1.54-1.45 (m, 1H), 1.41-1.31 (m, 3H), 1.14-1.04 (m, 3H), 0.67-0.52 (m, 3H). ¹⁹F NMR (376 MHz, DMSO-d6, ppm): δ −42.26 (3F), −73.46 (1.68F, TFA). |
| 74 | 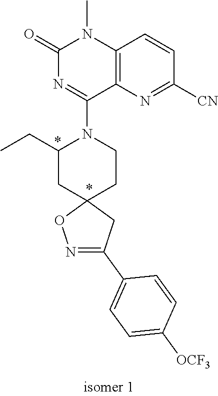 [SMILES: CCC1CC2(CCN1c1nc(=O)n(C)c3ccc(C#N)nc13)CC(c1ccc(OC(F)(F)F)cc1)=NO2] | 513.4 | SFC analysis: 92.77% ee; retention time: 4.358 min; column: AICELCHIRALCEL®AS, MeOH (0.1% of DEA) in CO2, 5% to 40%; pressure: 100 bar; flow rate: 70 mL/min; ¹H NMR (400 MHz, DMSO-d6, ppm) δ 8.31-8.20 (m, 1H), 8.08-7.95 (m, 1H), 7.89- 7.75 (m, 2H), 7.55-7.39 (m, 2H), 5.75-5.25 (m, 1H), 5.22-4.65 (m, 1H), 3.72-3.56 (m, 1H), 3.46 (s, 3H), 3.34-3.22 (m, 2H), 2.18- 1.73 (m, 6H), 1.04-0.85 (m, 3H). ¹⁹F NMR (376 MHz, DMSO- d6, ppm) δ −56.75 (3F) |
| 75 | 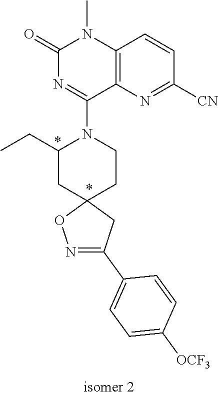 [SMILES: CCC1CC2(CCN1c1nc(=O)n(C)c3ccc(C#N)nc13)CC(c1ccc(OC(F)(F)F)cc1)=NO2] | 513.4 | SFC analysis: 93.15% ee; retention time: 4.424 min; column: DAICELCHIRALCEL®AS, MeOH (0.1% of DEA) in CO2, 5% to 40%; pressure: 100 bar; flow rate: 70 mL/min; ¹H NMR (400 MHz, DMSO-d6, ppm) δ 8.32-8.20 (m, 1H), 8.08-7.94 (m, 1H), 7.89- 7.73 (m, 2H), 7.55-7.40 (m, 2H), 5.72-5.28 (m, 1H), 5.24-4.60 (m, 1H), 3.74-3.58 (m, 1H), 3.46 (s, 3H), 3.34-3.24 (m, 2H), 2.19- 1.75 (m, 6H), 1.05-0.85 (m, 3H). ¹⁹F NMR (376 MHz, DMSO- d6, ppm) δ −56.75 (3F). |
| 76 | 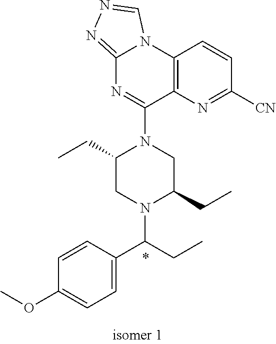 [SMILES: CCC(c1ccc(OC)cc1)N1C[C@H](CC)N(c2nc3nncn3c3ccc(C#N)nc23)C[C@H]1CC] | 485.4 | SFC analysis: 100% ee; retention time: 1.787 min; column: Chiralpak AS, 150 × 4.6 mm I.D., 3 μm, 40% of Methanol (0.05% DEA) in CO2; pressure: 100 bar; flow rate: 2.5 mL/min. ¹H NMR (400 MHz, DMSO-d6, ppm): δ 9.49 (s, 1H), 8.90 (d, J = 8.6 Hz, 1H), 8.54 (d, J = 8.6 Hz, 1H), 7.30-7.18 (m, 2H), 6.91-6.80 (m, 2H), 6.13- 4.32 (m, 2H), 3.74 (s, 3H), 3.62-3.40 (m, 1H), 3.35-3.25 (m, 1H), 3.22-3.05 (m, 1H), 2.60-2.50 (m, 1H), 2.41-2.30 (m, 1H), 2.12-1.71 (m, 3H), 1.68-1.39 (m, 3H), 1.06-0.92 (m, 3H), 0.71-0.55 (m, 6H). |
| 79 | 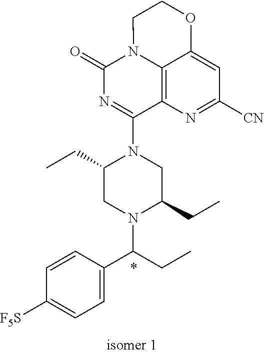 | 599.4 | SFC analysis: 99.12% ee; retention time: 4.630 min; column: DAICELCHIRALPAK@IG, EtOH (0.1% DEA) in CO2, 5% to 40%; pressure: 100 bar; flow rate: 1.5 mL/min. ¹H NMR (400 MHz, DMSO-d6, ppm): δ 7.90-7.81 (m, 3H), 7.58 (d, J = 7.8 Hz, 2H), 6.25- 5.37 (m, 1H), 5.16-4.66 (m, 1H), 4.62-4.36 (m, 2H), 4.12-3.85 (m, 2H), 3.65-3.51 (m, 1H), 3.46-3.40 (m, 1H), 3.13-3.10 (m, 1H), 2.15- 2.10 (m, 1H), 1.97-1.93 (m, 1H), 1.79-1.73 (m, 2H), 1.65-1.48 (m, 2H), 1.40-1.33 (m, 2H), 0.97-0.90 (m, 3H), 0.60-0.54 (m, 6H). ¹⁹F NMR (376 MHz, DMSO-d6, ppm): δ 87.95 (1F), 64.30 (4F). |
| 80 | 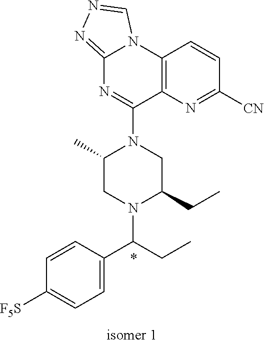 | 567.4 | Analytical HPLC*: retention time: 1.61 min; 0.41 FA salt, ¹H NMR (400 MHz, DMSO-d₆, ppm): δ 9.49 (s, 1H), 8.90 (d, J = 12.0 Hz, 1H), 8.54 (d, J = 8.8 Hz, 1H), 8.44 (s, 0.41H), 7.91 (d, J = 8.4 Hz, 2H), 7.58 (d, J = 8.4 Hz, 2H), 5.81-5.40 (m, 1H), 5.35-5.05 (m, 1H), 3.71-3.65 (m, 1H), 3.42-3.39 (m, 1H), 2.95-2.87 (m, 1H), 2.78-2.70 (m, 1H), 2.39-2.32 (m, 1H), 1.92-1.83 (m, 1H), 1.65-1.45 (m, 1H), 1.52-1.38 (m, 5H), 0.78-0.70 (m, 3H), 0.68-0.61 (m, 3H); ¹⁹F NMR (376 MHz, DMSO-d₆, ppm): δ 88.01 (1F), 64.44 (4F). |
| 81 | 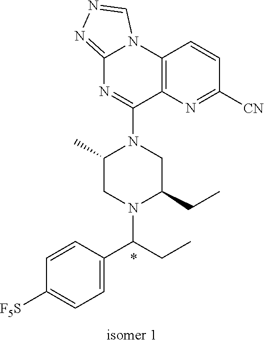 | 509.4 | SFC analysis: 100% ee; retention time: 1.835 min; column: Chiralpak AD-3 150 × 4.6 mm I.D., 3 um, 40% of Ethanol (0.05% of DEA) in CO2; pressure: 100 bar; flow rate: 2.5 mL/min; ¹H NMR (400 MHz, DMSO-d₆, ppm) δ 9.49 (s, 1H), ), 8.92-8.90 (d, J = 8.6 Hz, 1H), 8.56-8.54 (d, J = 8.6 Hz, 1H), 7.81-7.67 (m, 2H), 7.64- 7.50 (m, 2H), 5.85-4.75 (m, 2H), 3.75-3.60 (m, 1H), 3.48-3.36 (m, 1H), 3.00-2.86 (m, 1H), 2.82-2.68 (m, 1H), 2.43-2.31 (m, 1H), 2.00-1.80 (m, 1H), 1.71-1.57 (m, 1H), 1.56-1.39 (m, 5H), 0.80-0.55 (m, 6H). ¹⁹F NMR (376 MHz, DMSO-d₆, ppm) δ −60.68 (3F) |
| 82 | 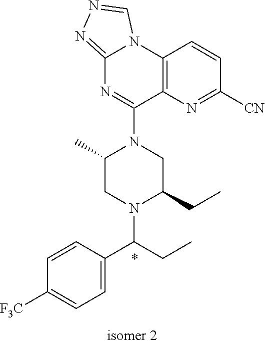 [SMILES: CCC(c1ccc(C(F)(F)F)cc1)N1C[C@H](C)N(c2nc3nncn3c3ccc(C#N)nc23)C[C@H]1CC] | 509.4 | SFC analysis: 100% ee; retention time: 2.269 min; column: Chiralpak AD-3 150 × 4.6 mm I.D., 3 um, 40% of Ethanol (0.05% of DEA) in CO2; pressure: 100 bar; flow rate: 2.5 mL/min; ¹H NMR (400 MHz, DMSO-d₆, ppm) δ 9.51 (s, 1H), 8.93-8.91 (d, J = 8.6 Hz, 1H), 8.57-8.55 (d, J = 8.6 Hz, 1H), 7.84-7.48 (m, 4H), 6.10-4.50 (m, 2H), 3.78-3.47 (m, 2H), 3.25-3.04 (m, 1H), 2.78-2.60 (m, 1H), 2.24-2.06 (m, 1H), 1.97-1.79 (m, 1H), 1.77-1.43 (m, 3H), 1.40-1.26 (m, 3H), 1.16-0.95 (m, 3H), 0.73-0.48 (m, 3H). ¹⁹F NMR (376 MHz, DMSO-d₆, ppm) δ −60.64 (3F). |
| 83 | 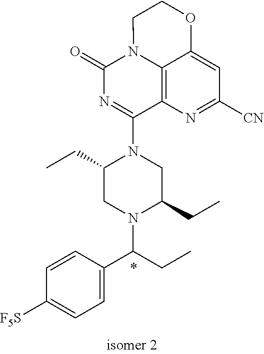 | 599.4 | SFC analysis: 100.00% ee; retention time: 4.302 min; column: DAICELCHIRALPAK@IG, EtOH (0.1% DEA) in CO2, 5% to 40%; pressure: 100 bar; flow rate: 1.5 mL/min. 0.29 TFA salt, 1H NMR (400 MHz, DMSO-d6, ppm): δ 8.01-7.74 (m, 3H), 7.60-7.51 (m, 2H), 6.08-5.52 (m, 1H), 5.08-4.81 (m, 1H), 4.43-4.42 (m, 2H), 4.02- 3.95 (m, 2H), 3.67-3.60 (m, 1H), 3.10-3.08 (m, 1H), 2.84-2.68 (m, 2H), 2.13-2.10 (m, 1H), 1.89-1.84 (m, 2H), 1.61-1.58 (m, 1H), 1.48- 1.44 (m, 1H), 1.27-1.22 (m, 2H), 0.95-0.92 (m, 3H), 0.66-0.61 (m, 6H). 19F NMR (376 MHz, DMSO-d6, ppm): δ 88.01 (1F), 64.44 (4F), −73.45 (0.86F, TFA). |
| 85 | 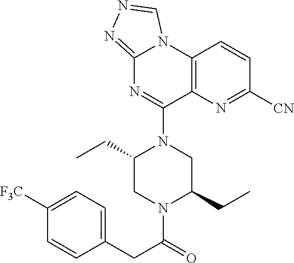 [SMILES: CC[C@@H]1CN(c2nc3nncn3c3ccc(C#N)nc23)[C@@H](CC)CN1C(=O)Cc1ccc(C(F)(F)F)cc1] | 523.4 | ¹H NMR (400 MHz, DMSO-d6, ppm) δ 9.54 (s, 1H), 8.99-8.89 (m, 1H), 8.63-8.54 (m, 1H), 7.74-7.65 (m, 2H), 7.57-7.47 (m, 2H), 5.87-4.35 (m, 3H), 4.20 (s, 0.5H), 4.11-4.01 (m, 0.5H), 3.98- 3.78 (m, 2H), 3.57-3.34 (m, 1.5H), 3.08-2.95 (m, 0.5H), 1.83- 1.50 (m, 4H), 0.98-0.73 (m, 6H). ¹⁹F NMR (376 MHz, DMSO-d6, ppm) δ −60.78 (3F). |
| 86 | 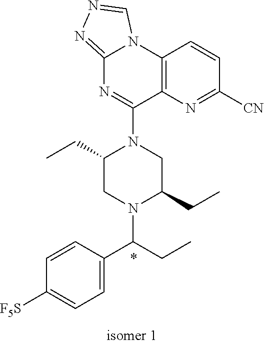 | 581.3 | SFC analysis: 100% ee; retention time: 1.497 min; column: ChiralPak AD, 250 × 30 mm, I.D., 10 μm, 5% to 35%; pressure: 100 bar; flow rate: 80 mL/min. ¹H NMR (400 MHz, DMSO-d6, ppm): δ 9.49 (s, 1H), 8.91 (d, J = 8.4 Hz, 1H), 8.56 (d, J = 8.4 Hz, 1H), 7.90 (d, J = 8.4 Hz, 2H), 7.58 (d, J = 8.4 Hz, 2H), 5.92-4.92 (m, 2H), 3.68-3.60 (m, 1H), 2.94-2.84 (m, 2H), 2.10-1.90 (m, 4H), 1.65-1.45 (m, 4H), 1.10-0.90 (m, 3H), 0.75-0.55 (m, 6H). ¹⁹F NMR (376 MHz, DMSO-d6, ppm): δ 88.41 (1F), 64.61 (4F). |
| 87 | 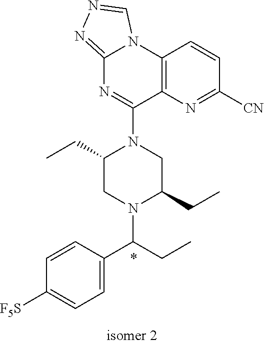 | 518.3 | SFC analysis: 98.16% ee; retention time: 2.193 min; column: ChiralPak AD, 250 × 30 mm, I.D., 10 μm, 5% to 35%; pressure: 100 bar; flow rate: 80 mL/min. ¹H NMR (400 MHz, DMSO-d₆, ppm): δ 9.50 (s, 1H), 8.91 (d, J = 8.4 Hz, 1H), 8.55 (d, J = 8.4 Hz, 1H), 7.88 (d, J = 8.4 Hz, 2H), 7.60 (d, J = 8.4 Hz, 2H), 5.92-4.60 (m, 2H), 3.54-3.53 (m, 2H), 3.18-3.09 (m, 1H), 2.20-1.97 (m, 4H), 1.85-1.45 (m, 4H), 1.10-0.90 (m, 3H), 0.75-0.55 (m, 6H). ¹⁹H NMR (376 MHz, DMSO-d6, ppm): δ 88.42 (1F), 64.57 (4F). |
| 88 | 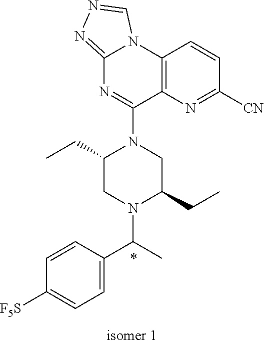 | 567.2 | SFC analysis: 100% ee; retention time: 1.618 min; column: Chiralpak AD, 150 × 4.6 mm I.D., 3 μm, 40% of ethanol (0.05% DEA) in CO₂; pressure: 100 bar; flow rate: 2.5 mL/min. ¹H NMR (400 MHz, DMSO-d₆, ppm): δ 9.49 (s, 1H), 8.91 (d, J = 8.8 Hz, 1H), 8.55 (d, J = 8.4 Hz, 1H), 7.90 (d, J = 8.4 Hz, 2H), 7.64 (d, J = 8.4 Hz, 2H), 5.92-4.72 (m, 2H), 3.93-3.81 (m, 1H), 2.99-2.79 (m, 2H), 2.34-2.12 (m, 2H), 1.98-1.87 (m, 2H), 1.58-1.40 (m, 2H), 1.30-1.25 (m, 3H), 0.97-0.90 (m, 3H) 0.72-0.64 (m, 3H). ¹⁹F NMR (376 MHz, DMSO- d₆, ppm): δ 88.42 (1F), 64.58 (4F). |
| 89 | 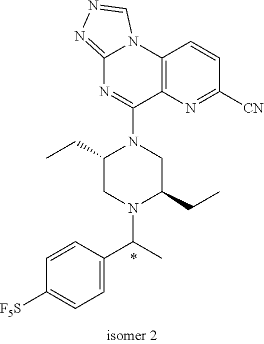 | 567.2 | SFC analysis: 99.08% ee; retention time: 2.515 min; column: Chiralpak AD, 150 × 4.6 mm I.D., 3 μm, 40% of ethanol (0.05% DEA) in CO₂; pressure: 100 bar; flow rate: 2.5 mL/min. ¹H NMR (400 MHz, DMSO-d₆, ppm): δ 9.50 (s, 1H), 8.91 (d, J = 8.8 Hz, 1H), 8.55 (d, J = 8.4 Hz, 1H), 7.90 (d, J = 8.4 Hz, 2H), 7.64 (d, J = 8.4 Hz, 2H), 5.96-4.42 (m, 2H), 3.75-3.68 (m, 1H), 3.60-3.48 (m, 1H), 3.18-3.10 (m, 1H), 2.34-2.22 (m, 2H), 2.05-1.87 (m, 2H), 1.58-1.40 (m, 2H), 1.28-1.21 (m, 3H), 1.05-0.98 (m, 3H), 0.68-0.56 (m, 3H). ¹⁹F NMR (376 MHz, DMSO-d₆, ppm): δ 88.45 (1F), 64.55 (4F). |
| 90 | 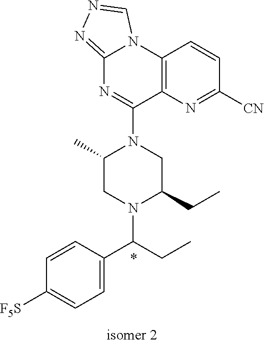 | 567.4 | Analytical HPLC*: retention time: 1.67 min; ¹H NMR (400 MHz, Chloroform-d, ppm) δ 9.12-8.89 (m, 2H), 8.47-8.32 (m, 1H), 8.19- 7.92 (m, 2H), 7.81-7.52 (m, 2H), 5.42-4.65 (m, 1H), 4.20-3.83 (m, 1H), 3.24-3.02 (m, 1H), 2.81-2.75 (m, 1H), 2.22-2.15 (m, 1H), 2.05- 1.95 (m, 1H), 1.82-1.79 (m, 1H), 1.70-1.67 (m, 1H), 1.48-1.41 (m, 1H), 1.38-1.30 (m, 4H), 1.29-1.18 (m, 3H), 1.12-1.06 (m, 1H), 0.71- 0.61 (m, 3H). ¹⁹F NMR (376 MHz, Chloroform-d, ppm): δ 82.75 (1F), 64.43 (4F). |
| 93 | 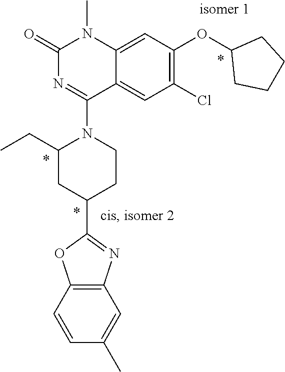 [SMILES: CCC1CC(c2nc3cc(C)ccc3o2)CCN1c1nc(=O)n(C)c2cc(OC3CCCC3)c(Cl)cc12] | 523.4 | SFC analysis: 100.00% ee; retention time: 4.770 min; column: ChiralPak AD, 250 × 30 mm I.D., 10 μm, A for CO₂ and B for Ethanol (0.1% NH₃H₂O), 50%; pressure: 100 bar; flow rate: 80 mL/min. ¹H NMR (400 MHz, DMSO-d₆, ppm): δ 7.77 (s, 1H), 7.60-7.51 (m, 2H), 7.19 (d, J = 8.4 Hz, 1H), 6.91 (s, 1H), 5.44-5.38 (m, 1H), 4.19-4.10 (m, 1H), 3.99-3.92 (m, 1H), 3.89-3.84 (m, 3H), 3.82-3.71 (m, 2H), 3.48 (s, 3H), 3.42-3.36 (m, 1H), 2.27 (s, 3H), 2.36-2.31 (m, 1H), 2.30-2.26 (m, 2H), 2.24-2.18 (m, 1H), 2.15-1.97 (m, 2H), 1.58-1.50 (m, 1H), 1.45-1.33 (m, 1H), 0.71 (t, J = 7.6 Hz, 3H). |
| 94 | 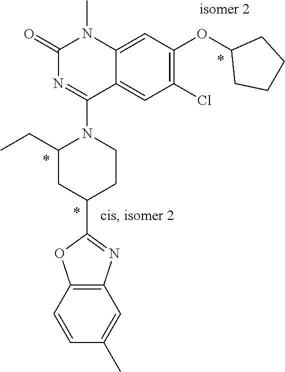 [SMILES: CCC1CC(c2nc3cc(C)ccc3o2)CCN1c1nc(=O)n(C)c2cc(OC3CCCC3)c(Cl)cc12] | 523.4 | SFC analysis: 100.00% ee; retention time: 6.152 min; column: ChiralPak AD, 250 × 30 mm I.D., 10 μm, A for CO₂ and B for Etha- nol (0.1% NH₃H₂O), 50%; pressure: 100 bar; flow rate: 80 mL/ min. ¹H NMR (400 MHz, DMSO-d₆, ppm): δ 7.77 (s, 1H), 7.57 (d, J = 8.0 Hz, 1H), 7.52 (s, 1H), 7.19 (d, J = 8.4 Hz, 1H), 6.91 (s, 1H), 5.44-5.38 (m, 1H), 4.19-4.10 (m, 1H), 3.99-3.93 (m, 1H), 3.92-3.84 (m, 3H), 3.80-3.72 (m, 2H), 3.49 (s, 3H), 3.42- 3.36 (m, 1H), 2.27 (s, 3H), 2.36-2.30 (m, 1H), 2.28-2.24 (m, 2H), 2.21-2.15 (m, 1H), 2.08-1.98 (m, 2H), 1.58-1.50 (m, 1H), 1.40-1.33 (m, 1H), 0.71 (t, J = 6.8 Hz, 3H). |
| 96 | 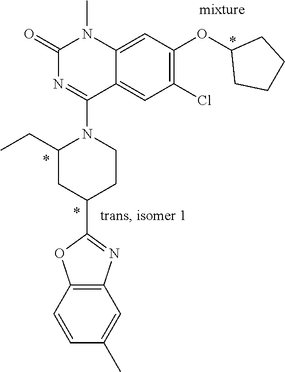 [SMILES: CCC1CC(c2nc3cc(C)ccc3o2)CCN1c1nc(=O)n(C)c2cc(OC3CCCC3)c(Cl)cc12] | 523.2 | Analytical HPLC*: retention time: 1.961 min; ¹H NMR (400 MHz, DMSO-d₆, ppm): δ 7.73 (s, 1H), 7.54 (d, J = 8.0 Hz, 1H), 7.47 (s, 1H), 7.16 (d, J = 8.0 Hz, 1H), 6.90 (s, 1H), 5.42 (s, 1H), 4.60-4.50 (m, 1H), 4.20-4.09 (m, 1H), 4.08-3.97 (m, 1H), 3.96-3.85 (m, 2H), 3.85-3.70 (m, 1H), 3.65-3.52 (m, 2H), 3.47 (s, 3H), 2.40-2.10 (m, 4H), 2.09-1.95 (m, 4H), 1.85-1.70 (m, 3H), 0.92-0.80 (m, 3H). |
| 97 | 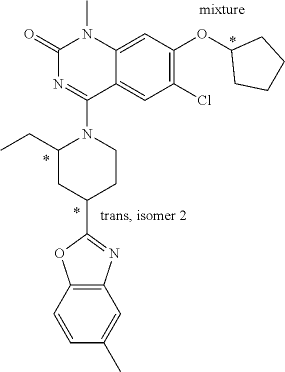 [SMILES: CCC1CC(c2nc3cc(C)ccc3o2)CCN1c1nc(=O)n(C)c2cc(OC3CCCC3)c(Cl)cc12] | 523.2 | Analytical HPLC*: retention time: 1.959 min; ¹H NMR (400 MHz, DMSO-d₆, ppm) δ 7.73 (s, 1H), 7.54 (d, J = 8.0 Hz, 1H), 7.47 (s, 1H), 7.16 (d, J = 8.0 Hz, 1H), 6.90 (s, 1H), 5.42 (s, 1H), 4.61-4.50 (m, 1H), 4.18-4.06 (m, 1H), 4.05-3.94 (m, 1H), 3.93-3.82 (m, 2H), 3.81-3.70 (m, 1H), 3.65-3.55 (m, 2H), 3.47 (s, 3H), 2.40-2.33 (m, 3H), 2.20-2.00 (m, 6H), 1.90-1.75 (m, 2H), 0.95-0.80 (m, 3H). |
| 100 | 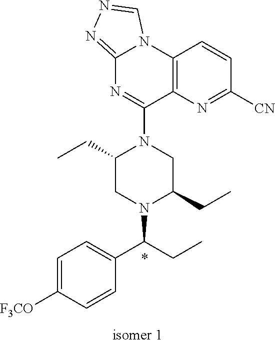 [SMILES: CC[C@H]1CN([C@@H](CC)c2ccc(OC(F)(F)F)cc2)[C@H](CC)CN1c1nc2nncn2c2ccc(C#N)nc12] | 539.3 | SFC analysis: 100% ee; retention time: 1.502 min; column: ChiralPak AD, 150 × 4.6 mm I.D., 3 μm, 40% of ethanol (0.05% DEA) in CO2; pressure: 100 bar; flow rate: 2.5 mL/min. ¹H NMR (400 MHz, Methanol-d4, ppm) δ 9.33 (s, 1H), 8.79-8.77 (d, J = 8.7 Hz, 1H), 8.33-8.31 (d, J = 8.6 Hz, 1H), 7.49-7.47 (d, J = 8.5 Hz, 2H), 7.31-7.29 (d, J = 8.1 Hz, 2H), 6.27-4.90 (m, 2H), 3.80-3.59 (m, 1H), 3.56-3.34 (m, 1H), 3.20-2.90 (m, 2H), 2.66-2.48 (m, 1H), 2.33-1.95 (m, 3H), 1.79-1.46 (m, 3H), 1.15-1.00 (m, 3H), 0.84- 0.64 (m, 6H). 19F NMR (376 MHz, Methanol-d4, ppm) δ −59.49 (3F). |
| 101 | 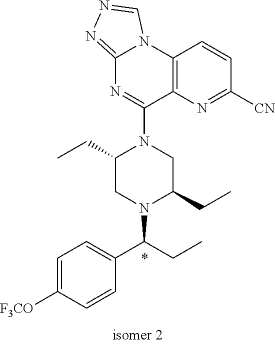 [SMILES: CC[C@H]1CN([C@@H](CC)c2ccc(OC(F)(F)F)cc2)[C@H](CC)CN1c1nc2nncn2c2ccc(C#N)nc12] | 539.3 | SFC analysis: 100% ee; retention time: 1.901 min; column: ChiralPak AD, 150 × 4.6 mm I.D., 3 μm, 40% of ethanol (0.05% DEA) in CO2; pressure: 100 bar; flow rate: 2.5 mL/min. ¹H NMR (400 MHz, Methanol-d4, ppm) δ 9.32 (s, 1H), 8.76 (d, J = 8.7 Hz, 1H), 8.30 (d, J = 7.4 Hz, 1H), 7.48 (d, J = 8.6 Hz, 2H), 7.25 (d, J = 8.1 Hz, 2H), 6.43-4.62 (m, 2H), 3.85-3.48 (m, 1H), 3.47-3.40 (m, 1H), 3.28-3.18 (m, 1H), 2.70-2.60 (m, 1H), 2.42-2.31 (m, 1H), 2.22- 1.78 (m, 3H), 1.76-1.50 (m, 3H), 1.18-1.04 (m, 3H), 0.72-0.63 (m, 6H). ¹⁹F NMR (376 MHz, Methanol-d4, ppm) δ −59.57 (3F). |
| 105 | 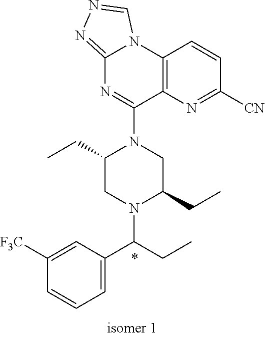 [SMILES: CCC(c1cccc(C(F)(F)F)c1)N1C[C@H](CC)N(c2nc3nncn3c3ccc(C#N)nc23)C[C@H]1CC] | 523.4 | SFC analysis: 100% ee; retention time: 1.328 min; column: Chiral- MIC, EtOH (0.05% of DEA) in CO₂, 5% to 40%; pressure: 100 bar; flow rate: 2.5 mL/min. ¹H NMR (400 MHz, DMSO-d₆, ppm): δ 9.49 (s, 1H), 8.91-8.89 (m, 1H), 8.56-8.53 (m, 1H), 7.69-7.60 (m, 4H), 5.60-4.80 (m, 2H), 3.70-3.66 (m, 1H), 3.31-3.27 (m, 1H), 2.96-2.93 (m, 1H), 2.86-2.82 (m, 1H), 2.33-2.32 (m, 1H), 2.09-2.08 (m, 1H), 2.00-1.88 (m, 2H), 1.65-1.58 (m, 1H), 1.47-1.43 (m, 2H), 0.97 (t, J = 7.6 Hz, 3H), 0.66-0.62 (m, 6H). ¹⁹F NMR (376 MHz, DMSO-d₆, ppm): δ −60.88 (3F). |
| 106 | 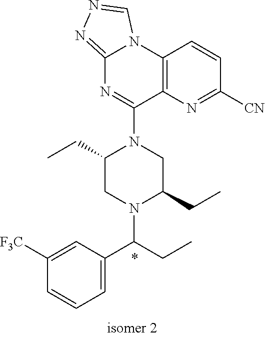 [SMILES: CCC(c1cccc(C(F)(F)F)c1)N1C[C@H](CC)N(c2nc3nncn3c3ccc(C#N)nc23)C[C@H]1CC] | 523.4 | SFC analysis: 97.52% ee; retention time: 1.581 min; column: Chiral- MIC, EtOH (0.05% of DEA) in CO₂, 5% to 40%; pressure: 100 bar; flow rate: 2.5 mL/min. ¹H NMR (400 MHz, DMSO-d₆, ppm): δ 9.50 (s, 1H), 8.92-8.89 (m, 1H), 8.56-8.53 (m, 1H), 7.65-7.63 (m, 1H), 7.60-7.56 (m, 3H), 5.91-4.56 (m, 2H), 3.57-3.54 (m, 2H), 3.20-3.11 (m, 1H), 2.67-2.56 (m, 1H), 2.33-2.32 (m, 1H), 2.23-2.20 (m, 1H), 1.89-1.73 (m, 2H), 1.63-1.45 (m, 3H), 1.03 (t, J = 7.2 Hz, 3H), 0.62- 0.57 (m, 6H). ¹⁹F NMR (376 MHz, DMSO-d₆, ppm): δ −61.0 (3F). |
| 111 | 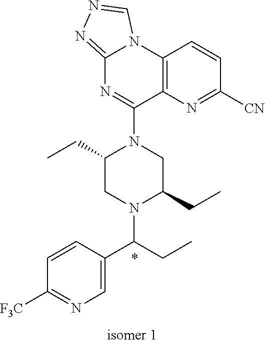 [SMILES: CCC(c1ccc(C(F)(F)F)nc1)N1C[C@H](CC)N(c2nc3nncn3c3ccc(C#N)nc23)C[C@H]1CC] | 524.2 | Analytical HPLC*: retention time: 1.468 min; 2.4 FA salt, ¹H NMR (400 MHz, DMSO-d₆, ppm): δ 9.48 (s, 1H), 8.91 (d, J = 8.4 Hz, 1H), 8.75 (s, 1H), 8.54 (d, J = 8.8 Hz, 1H), 8.46 (s, 2.4H), 8.08 (d, J = 8.0 Hz, 1H), 7.93 (d, J = 8.0 Hz, 1H), 5.95-4.84 (m, 2H), 3.82-3.72 (m, 1H), 2.94-2.82 (m, 2H), 2.04-1.89 (m, 4H), 1.73-1.62 (m, 2 H), 1.54- 1.44 (m, 2H), 0.99-0.89 (m, 3H), 0.75-0.59 (m, 6H). ¹⁹F NMR (376 MHz, DMSO-d₆, ppm): δ −66.10 (3F). |
| 112 | 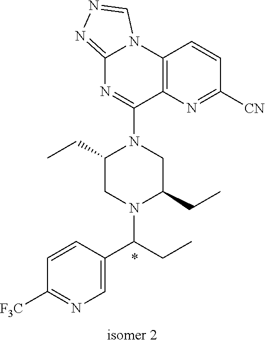 [SMILES: CCC(c1ccc(C(F)(F)F)nc1)N1C[C@H](CC)N(c2nc3nncn3c3ccc(C#N)nc23)C[C@H]1CC] | 524.2 | Analytical HPLC*: retention time: 1.530 min; 3.5 FA salt, ¹H NMR (400 MHz, DMSO-d₆, ppm): δ 9.49 (s, 1H), 8.91 (d, J = 8.4 Hz, 1H), 8.75 (s, 1H), 8.54 (d, J = 8.4 Hz, 1H), 8.46 (s, 3.5H), 8.08 (d, J = 8.0 Hz, 1H), 7.92 (d, J = 8.0 Hz, 1H), 5.92-4.33 (m, 2H), 3.75-3.66 (m, 1H), 2.26-2.18 (m, 2H), 2.04-1.89 (m, 4H), 1.69-1.58 (m, 2H), 1.48- 1.38 (m, 2H), 1.06-0.95 (m, 3H), 0.69-0.58 (m, 6H). ¹⁹F NMR (376 MHz, DMSO-d₆, ppm): δ −66.11 (3F). |
| 115 | 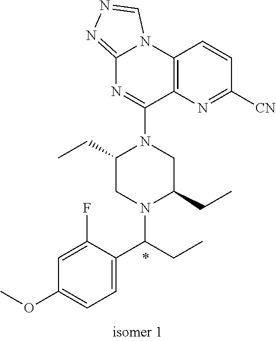 [SMILES: CCC(c1ccc(OC)cc1F)N1C[C@H](CC)N(c2nc3nncn3c3ccc(C#N)nc23)C[C@H]1CC] | 503.4 | Analytical HPLC*: retention time: 1.342 min; ¹H NMR (400 MHz, DMSO-d₆, ppm) δ 9.48 (s, 1H), 8.90 (d, J = 8.8 Hz, 1H), 8.54 (d, J = 8.8 Hz, 1H), 7.38 (t, J = 8.4 Hz, 1H), 6.93-6.76 (m, 2H), 5.55-4.75 (m, 2H), 3.88-3.83 (m, 1H), 3.78 (s, 3H), 3.35-3.28 (s, 1H) ,2.95- 2.82 (m, 1H), 2.80-2.71 (m, 1H), 2.42-2.38 (m, 1H), 2.09-1.79 (m, 3H), 1.67-1.53 (m, 1H), 1.50-1.34 (m, 2H), 0.96 (t, J = 7.6 Hz, 3H), 0.72-0.62 (m, 6H). ¹⁹F NMR (376 MHz, DMSO-d₆, ppm): δ −116.84 (1F). |
| 116 | 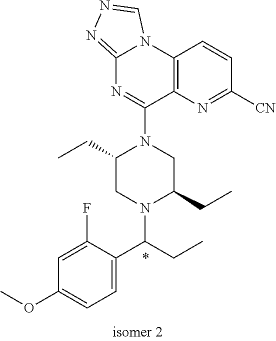 [SMILES: CCC(c1ccc(OC)cc1F)N1C[C@H](CC)N(c2nc3nncn3c3ccc(C#N)nc23)C[C@H]1CC] | 503.4 | Analytical HPLC*: retention time: 1.377 min; ¹H NMR (400 MHz, DMSO-d₆, ppm) δ 9.49 (s, 1H), 8.90 (d, J = 8.4 Hz, 1H), 8.54 (d, J = 8.4 Hz, 1H), 7.43 (t, J = 8.4 Hz, 1H), 6.88-6.72 (m, 2H), 5.91-5.62 (m, 1H), 4.98-4.40 (m, 1H), 3.82-3.66 (m, 4H), 3.58-3.42 (m, 1H), 3.20-3.11 (m, 1H), 2.63-2.54 (m, 1H), 2.42-2.27 (m, 1H), 1.99-1.93 (m, 1H), 1.90-1.69 (m, 2H), 1.66-1.47 (m, 2H), 1.47-1.32 (m, 1H), 1.00 (t, J = 7.2 Hz, 3H), 0.66 (t, J = 7.2 Hz, 6H). ¹⁹F NMR (376 MHz, DMSO-d₆, ppm): δ −117.18 (1F). |
| 117 | 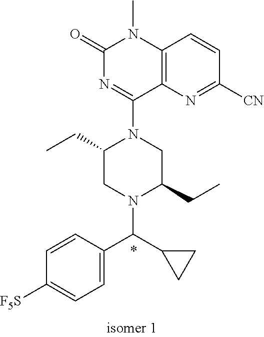 | 583.4 | SFC analysis: 100% ee; retention time: 1.120 min; column: Chiralpak AD, 150 × 4.6 mm I.D., 3 μm, 40% of ethanol (0.05% DEA) in CO2; pressure: 100 bar; flow rate: 2.5 mL/min. ¹H NMR (400 MHz, DMSO-d6, ppm): δ 8.28-8.19 (m, 1H), 7.99 (d, J = 9.0 Hz, 1H), 7.92-7.85 (m, 2H), 7.66-7.58 (m, 2H), 5.83-5.71 (m, 0.5H), 5.63-5.51 (m, 0.5H), 5.09-5.00 (m, 0.5H), 4.92-4.80 (m, 0.5H), 3.52-3.40 (m, 4.5H), 3.15-3.07 (m, 0.5H), 2.85-2.65 (m, 2H), 2.31-1.90 (m, 3H), 1.45-1.31 (m, 2H), 1.05-0.92 (m, 3H), 0.91-0.83 (m, 1H), 0.82-0.74 (m, 1H), 0.73-0.51 (m, 3H), 0.48-0.23 (m, 2H), 0.08-0.00 (m, 1H); ¹⁹F NMR (376 MHz, DMSO-d6, ppm): δ 88.11 (1F), 64.46 (4F). |
| 118 | 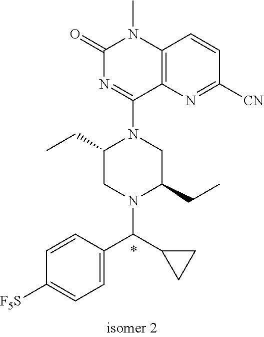 | 583.4 | SFC analysis: 99.72% ee; retention time: 1.517 min; column: Chiralpak AD, 150 × 4.6 mm I.D., 3 μm, 40% of ethanol (0.05% DEA) in CO₂; pressure: 100 bar; flow rate: 2.5 mL/min. ¹H NMR (400 MHz, DMSO-d6, ppm): δ 8.30- 8.17 (m, 1H), 8.05-7.93 (m, 1H), 7.91-7.83 (m, 2H), 7.64-7.55 (m, 2H), 6.14-6.05 (m, 0.5H), 5.29-5.20 (m, 0.5H), 5.16-5.07 (m, 0.5H), 4.80-4.71 (m, 0.5H), 3.71-3.63 (m, 0.5H), 3.62-3.50 (m, 1H), 3.43 (s, 3H), 3.39-3.35 (m, 0.5H), 2.92- 2.84 (m, 1H), 2.70-2.64 (m, 0.5H), 2.35-2.31 (m, 0.5H), 2.11-1.75 (m, 3H), 1.55-1.41 (m, 2H), 1.05-0.90 (m, 4H), 0.84-0.75 (m, 1H), 0.65-0.55 (m, 3H), 0.51-0.22 (m, 2H), 0.06-(−0.06) (m, 1H); ¹⁹F NMR (376 MHz, DMSO-d6, ppm): δ 88.09 (1F), 64.40 (4F). |
| 125 | 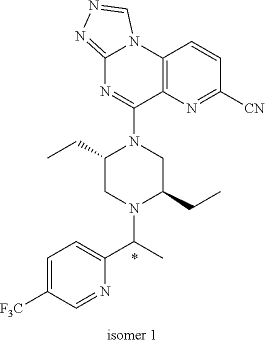 [SMILES: CC[C@H]1CN(C(C)c2ccc(C(F)(F)F)cn2)[C@H](CC)CN1c1nc2nncn2c2ccc(C#N)nc12] | 524.2 | SFC analysis: 100% ee; retention time: 1.504 min; column: Chiralpak AD, 150 × 4.6 mm I.D., 3 μm, 40% of ethanol (0.05% DEA) in CO₂; pressure: 100 bar; flow rate: 2.5 mL/min 0.19 TFA salt, ¹H NMR (400 MHz, DMSO-d6, ppm): δ 9.52 (s, 1H), 9.06-8.96 (m, 1H), 8.95-8.85 (m, 1H), 8.61-8.50 (m, 1H), 8.31-8.20 (m, 1H), 7.80-7.65 (m, 1H), 5.00-4.60 (m, 2H), 4.00-3.81 (m, 1H), 3.59-3.38 (m, 2H), 3.12-2.75 (m, 2H), 2.05-1.81 (m, 4H), 1.59-1.35 (m, 2H), 1.00-0.89 (m, 3H), 0.80-0.58 (m, 6H). ¹⁹F NMR (376 MHz, DMSO-d₆, ppm): δ −60.64 (3F), −73.48 (0.58F, TFA). |
| 126 | 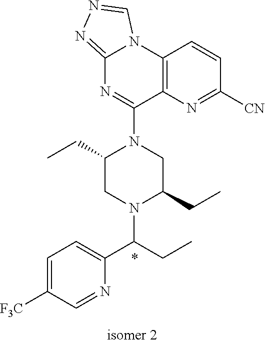 [SMILES: CCC(c1ccc(C(F)(F)F)cn1)N1C[C@H](CC)N(c2nc3nncn3c3ccc(C#N)nc23)C[C@H]1CC] | 524.2 | SFC analysis: 98.66% ee; retention time: 2.142 min; column: Chiralpak AD, 150 × 4.6 mm I.D., 3 μm, 40% of ethanol (0.05% DEA) in CO₂; pressure: 100 bar; flow rate: 2.5 mL/min. 0.85 TFA salt, ¹H NMR (400 MHz, DMSO-d₆, ppm): δ 9.56 (s, 1H), 9.05-8.85 (m, 2H), 8.64-8.52 (m, 1H), 8.40-8.12 (m, 1H), 7.85-7.69 (m, 1H), 5.60-4.05 (m, 2H), 3.89-3.61 (m, 2H), 3.25-2.72 (m, 3H), 2.05-1.85 (m, 4H), 1.68-1.51 (m, 2H), 1.07-0.96 (m, 3H), 0.72-0.60 (m, 6H). ¹⁹F NMR (376 MHz, DMSO-d₆, ppm): δ −60.64 (3F), −73.47 (2.55F, TFA). |
| 127 | 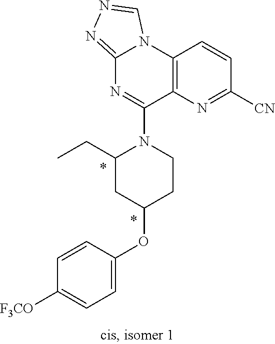 [SMILES: CCC1CC(Oc2ccc(OC(F)(F)F)cc2)CCN1c1nc2nncn2c2ccc(C#N)nc12] | 484.2 | SFC analysis: 100% ee; retention time: 2.922 min; column: ChiralPak AD, 150 × 4.6 mm, I.D., 3 μm, Methanol (0.05% DEA)/CO₂ = 40/60; pressure: 100 bar; flow rate: 2.5 mL/min. ¹H NMR (400 MHz, DMSO-d₆, ppm): δ 9.53 (s, 1H), 8.94-8.91 (d, J = 8.8 Hz, 1H), 8.57-8.55 (d, J = 8.8 Hz, 1H), 7.33-7.31 (m, 2H), 7.12-7.09 (m, 2H), 5.14 (s, 1H), 4.85 (s, 1H), 4.77-4.75 (m, 1H), 3.68-3.61 (m, 1H), 2.19-1.98 (m, 6H), 0.89-0.85 (m, 3H). ¹⁹F NMR (376 MHz, DMSO-d₆, ppm): δ −57.23 (3F). |
| 128 | 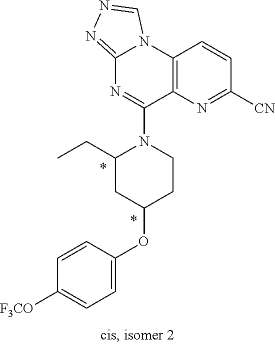 [SMILES: CCC1CC(Oc2ccc(OC(F)(F)F)cc2)CCN1c1nc2nncn2c2ccc(C#N)nc12] | 484.2 | SFC analysis: 100% ee; retention time: 4.037 min; column: ChiralPak AD, 150 × 4.6 mm, I.D., 3 μm, Methanol (0.05% DEA)/CO₂ = 40/60; pressure: 100 bar; flow rate: 2.5 mL/min. ¹H NMR (400 MHz, DMSO-d₆, ppm): δ 9.53 (s, 1H), 8.94-8.91 (d, J = 8.8 Hz, 1H), 8.57-8.55 (d, J = 8.8 Hz, 1H), 7.33-7.31 (m, 2H), 7.12-7.09 (m, 2H), 5.14 (s, 1H), 4.85 (s, 1H), 4.76-4.74 (m, 1H), 3.68-3.62 (m, 1H), 2.21-1.98 (m, 6H), 0.89-0.85 (m, 3H). ¹⁹F NMR (376 MHz, DMSO-d₆, ppm): δ −57.24 (3F). |
| 129 | 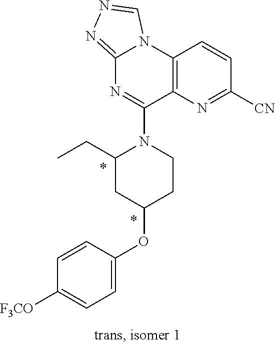 [SMILES: CCC1CC(Oc2ccc(OC(F)(F)F)cc2)CCN1c1nc2nncn2c2ccc(C#N)nc12] | 484.2 | SFC analysis: 100% ee; retention time: 2.37 min; column: ChiralPak AD, 150 × 4.6 mm I.D., 3 μm, Ethanol (0.05% DEA)/CO₂ = 40/60; pressure: 100 bar; flow rate: 2.5 mL/min. ¹H NMR (400 MHz, DMSO-d₆, ppm): δ 9.55 (s, 1H), 8.94-8.92 (d, J = 8.8 Hz, 1H), 8.58- 8.55 (d, J = 8.8 Hz, 1H), 7.31-7.29 (m, 2H), 7.12-7.09 (m, 2H), 5.36 (s, 1H), 4.95-4.90 (m, 2H), 3.49-3.42 (m, 1H), 2.26-1.72 (m, 6H), 0.99-0.95 (m, 3H). ¹⁹F NMR (376 MHz, DMSO-d₆, ppm): δ −57.25 (3F). |
| 130 | 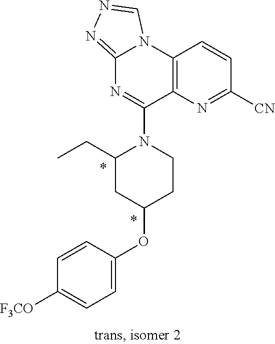 [SMILES: CCC1CC(Oc2ccc(OC(F)(F)F)cc2)CCN1c1nc2nncn2c2ccc(C#N)nc12] | 484.2 | SFC analysis: 100% ee; retention time: 3.168 min; column: ChiralPak AD, 150 × 4.6 mm, I.D., 3 μm, Ethanol (0.05% DEA)/CO₂ = 40/60; pressure: 100 bar; flow rate: 2.5 mL/min. ¹H NMR (400 MHz, DMSO-d₆, ppm): δ 9.55 (s, 1H), 8.94-8.92 (d, J = 8.8 Hz, 1H), 8.58-8.55 (d, J = 8.8 Hz, 1H), 7.31-7.29 (m, 2H), 7.12-7.09 (m, 2H), 5.36 (s, 1H), 4.96-4.90 (m, 2H), 3.50-3.43 (m, 1H), 2.26-1.71 (m, 6H), 0.99-0.91 (m, 3H). ¹⁹F NMR (376 MHz, DMSO-d₆, ppm): δ −57.25 (3F). |
| 131 | 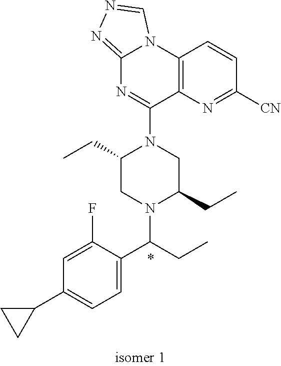 [SMILES: CCC(c1ccc(C2CC2)cc1F)N1C[C@H](CC)N(c2nc3nncn3c3ccc(C#N)nc23)C[C@H]1CC] | 513.3 | SFC analysis: 100% ee; retention time: 2.631 min; column: ChiralPak AD, 100 × 4.6 mm, I.D., 3 μm, Methanol (0.05% DEA)/CO2 = 40/60; pressure: 100 bar; flow rate: 2.5 mL/min. ¹H NMR (400 MHz, DMSO-d₆, ppm): δ 9.48 (s, 1H), 8.92-8.89 (d, J = 8.8 Hz, 1H), 8.56- 8.54 (d, J = 8.8 Hz, 1H), 7.35-7.32 (m, 1H), 6.98-6.87 (m, 2H), 5.45- 4.75 (m, 2H), 3.88-3.84 (m, 1H), 3.33 (m, 1H), 2.91-2.79 (m, 2H), 2.50-2.40 (m, 1H), 1.97-1.84 (m, 4H), 1.63-1.59 (m, 1H), 1.43-1.39 (m, 2H), 1.0-0.94 (m, 5H), 0.74-0.66 (m, 4H). ¹⁹F NMR (376 MHz, DMSO-d₆, ppm): δ −119.55 (3F). |
| 132 | 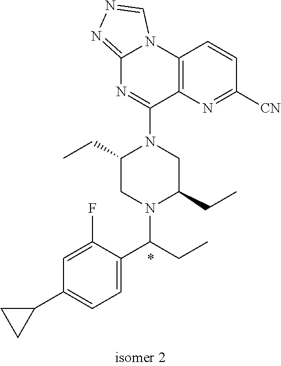 [SMILES: CCC(c1ccc(C2CC2)cc1F)N1C[C@H](CC)N(c2nc3nncn3c3ccc(C#N)nc23)C[C@H]1CC] | 513.4 | SFC analysis: 100% ee; retention time: 2.631 min; column: ChiralPak AD, 100 × 4.6 mm, I.D., 3 μm, Methanol (0.05% DEA)/CO2 = 40/60; pressure: 100 bar; flow rate: 2.5 mL/min. ¹H NMR (400 MHz, DMSO-d₆, ppm): δ 9.49 (s, 1H), 8.92-8.90 (d, J = 8.8 Hz, 1H), 8.56- 8.53 (d, J = 8.8 Hz, 1H), 7.41-7.37 (m, 1H), 6.98-6.82 (m, 2H), 5.75- 4.65 (m, 2H), 3.76-3.73 (m, 1H), 3.33 (m, 1H), 3.11-3.01 (m, 1H), 2.58-2.55 (m, 1H), 2.35-2.32 (m, 1H), 1.96-1.78 (m, 4H), 1.59-1.44 (m, 3H), 1.01-0.95 (m, 5H), 0.71-0.63 (m, 8H). ¹⁹F NMR (376 MHz, DMSO-d₆, ppm): δ −119.81 (3F). |
| 133 | 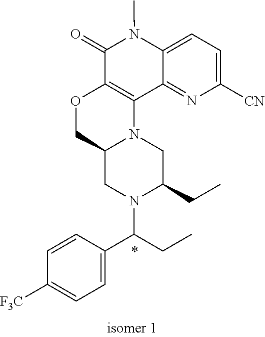 [SMILES: CCC(c1ccc(C(F)(F)F)cc1)N1C[C@H]2COc3c(c4nc(C#N)ccc4n(C)c3=O)N2C[C@H]1CC] | 498.2 | Analytical HPLC*: retention time: 1.504 min; ¹H NMR (400 MHz, DMSO-d₆, ppm) δ 8.08 (s, 2H), 7.72-7.62 (m, 4H), 5.15-5.12 (m, 1H), 4.20-4.18 (m, 1H), 3.95-3.75 (m, 2H), 3.58 (s, 3H), 3.18-2.93 (m, 2H), 2.39-2.36 (m, 2H), 1.76-1.55 (m, 2H), 1.35-1.24 (m, 4H), 1.13-1.03 (m, 3H). ¹⁹F NMR (376 MHz, DMSO-d₆, ppm) δ −60.68 (s, 3F). |
| 134 | 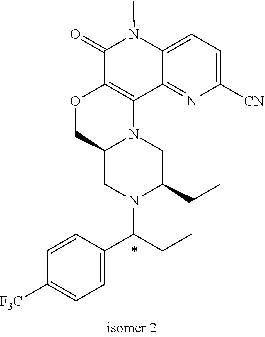 [SMILES: CCC(c1ccc(C(F)(F)F)cc1)N1C[C@H]2COc3c(c4nc(C#N)ccc4n(C)c3=O)N2C[C@H]1CC] | 498.2 | Analytical HPLC*: retention time: 1.501 min; ¹H NMR (400 MHz, DMSO-d₆, ppm) δ 8.06 (s, 2H), 7.72-7.59 (m, 4H), 5.03-4.99 (m, 1H), 4.37-4.34 (m, 1H), 4.00-3.95 (m, 2H), 3.59 (s, 3H), 3.34 (m, 1H), 2.99-2.94 (m, 2H), 2.58-2.55 (m, 1H), 2.41-2.39 (m, 1H), 1.70- 1.56 (m, 2H), 1.32-1.30 (m, 3H), 0.78 (m, 3H). ¹⁹F NMR (376 MHz, DMSO-d₆, ppm) δ −60.73 (s, 3F). |

*Analytical HPLC: column: Waters ACQUITY BEH C18 2.1*50 mm, 1.7 um; Mobile phaseA: H₂O (0.05% TFA); Mobile phaseB: Acetonitrile (0.05% TFA); flow rate: 1.0 mL/min; Run time: 5 min; 95 to 5% A in 3 min, 5% A for 2 min.

### Biological Assay Example A: DGK ADP-Glo Kinase Assay

The DGKa and DGKz kinase assays were performed using Promega ADP-Glo kit (Promega, Cat #V9102). One microliter test compound was added to a 384-well plate. Reactions were performed in a 5 uL volume by adding 2 uL mixture of DGK enzyme and 37.5 uM ATP in assay buffer (50 mM MOPS pH 7.5, 100 mM NaCl, 10 mM MgCl₂, 1 mM CaCl₂, 0.0025% BSA, and 1 mM DTT). The final concentration of DGK a, DGKz and ATP were 10 nM, 5 nM and 15 uM. The enzyme solution was incubated with test compound at 25° C. for 20 min. For the preparation of the substrate of micelles, 1 volume of a 16.1 mM solution of 1,2-dioleoyl-sn-glycerol in chloroform was slowly evaporated using a nitrogen stream. Subsequently, 22.55 volumes of a 510 mM solution of octyl-b-D-glucopyranoside in 50 mM MOPS buffer (pH 7.5) were added, and the mixture was sonicated in an ultrasonic bath for 20 s. Then 35 volumes of 50 mM MOPS buffer (pH7.5) were added to yield a solution of 0.28 mM 1,2 dioleoyl-sn-glycerol and 200 mM octyl-b-D-glucopyranoside, which was aliquoted, flash-frozen in liquid nitrogen, and stored at −80° C. until use. The reaction was initiated by the addition of 2 μL of substrate solution (7 uM 1.2-dioleoyl-sn-glycerol, 5 mM octyl-b-D-glucopyranoside in the 5 ul assay volume). The resulting mixture was incubated at 25° C. for 40 minutes. Next, 5 ul of ADP-Glo-reagent was added to the plate and incubated for 40 minutes. Then 10 ul of kinase detection reagent was added and incubated for 30 minutes. Luminescence was recorded using an Tecan SPARK microplate reader. 1% DMSO vehicle was used as control and no enzyme well was used as blank well. The percent inhibition was calculated with the formula: % inhibition=100-100*(RLU_{cmpd}−RLU_{blank})/(RLU_{control}−RLU_{blank}). Inhibition at 50% activity (IC50) was calculated with the equation of Y=Bottom+(Top−Bottom)/(1+10\hat{ }((LogIC50−X)*Hill Slope))

Table 2 below shows IC50 values measured and/or calculated according to this biological example.

TABLE 2
Kinase Inhibition of Representative Compounds (IC₅₀)
(A: <100 nM; B: 100 nM-1 uM (micromolar); C: >1 uM)

| | DGKa kinase | DGKz kinase |
|---|---|---|
| Compound # | IC_{50 (nM)} | IC_{50 (nM)} |
| 5 | B | B |
| 6 | B | B |
| 7 | B | B |
| 8 | B | B |
| 9 | A | C |
| 10 | B | B |
| 11 | A | C |
| 12 | C | C |
| 13 | B | C |
| 14 | C | C |
| 15 | A | C |
| 16 | B | A |
| 17 | A | B |
| 18 | A | B |
| 19 | C | A |
| 20 | C | C |
| 21 | A | C |
| 22 | A | C |
| 23 | A | C |
| 24 | A | C |
| 25 | A | B |
| 26 | A | B |
| 27 | A | B |
| 28 | A | A |
| 29 | A | A |
| 30 | A | A |
| 31 | A | A |
| 32 | A | C |
| 33 | A | A |
| 34 | A | A |
| 35 | A | B |
| 36 | A | B |
| 37 | A | B |
| 38 | A | B |
| 39 | A | C |
| 40 | A | B |
| 41 | A | B |
| 42 | A | B |
| 43 | A | B |
| 44 | A | B |
| 45 | B | A |
| 46 | A | B |
| 47 | A | A |
| 48 | A | B |
| 49 | A | A |
| 50 | A | C |
| 51 | A | A |
| 52 | A | B |
| 53 | A | B |
| 54 | A | B |
| 55 | A | A |
| 56 | B | B |
| 57 | A | B |
| 58 | A | B |
| 59 | A | B |
| 60 | A | A |
| 61 | B | B |
| 62 | A | B |
| 63 | A | B |
| 64 | A | A |
| 65 | A | A |
| 66 | A | A |
| 67 | A | A |
| 68 | A | B |
| 69 | A | B |
| 70 | A | A |
| 71 | A | B |
| 72 | A | A |
| 73 | A | A |
| 74 | A | C |
| 75 | B | B |
| 77 | A | A |
| 78 | A | A |
| 79 | B | B |
| 80 | A | A |
| 81 | A | A |
| 82 | A | B |
| 83 | A | B |
| 84 | A | C |
| 85 | A | B |
| 86 | A | A |
| 87 | A | A |
| 88 | A | A |
| 89 | A | B |
| 90 | A | B |
| 91 | A | C |
| 92 | B | A |
| 93 | A | B |
| 94 | B | C |
| 95 | A | B |
| 96 | A | C |
| 97 | A | A |
| 98 | A | A |
| 99 | A | A |
| 100 | A | A |
| 101 | A | B |
| 102 | A | C |
| 103 | A | B |
| 104 | A | A |
| 105 | A | A |
| 106 | A | B |
| 107 | A | B |
| 108 | A | B |
| 109 | A | A |
| 110 | A | B |
| 111 | A | A |
| 112 | A | B |
| 113 | A | B |
| 114 | A | A |
| 115 | A | A |
| 116 | A | B |
| 117 | A | B |
| 118 | A | B |
| 119 | A | A |
| 120 | A | B |
| 121 | A | C |
| 122 | A | A |
| 123 | A | B |
| 124 | A | B |

### Biological Assay Example B: Human PBMC IL-2 Assay

PBMC (frozen) obtained from health donor were thawed in RPMI-1640 medium with 10% o FBS, incubated for 4 h at 37° C. In a 96-well plate, 200,000 PBMC cells in 160 uL were added to each assay well. Ten microliter compound solution (10 uM to 0.6 nM, 8 points with 4-fold dilution) were added to cells in each well and incubated for 60 minutes at 37° C. Then 30 uL anti-CD3/CD28 antibody (Catalog #10991, Stemcell) were added to cells and incubated overnight at 37° C. After the incubation, 16 uL supernatants were transferred into a new 384-well white assay plate for cytokine analysis. IL-2 level was quantified using Human IL-2 kit (Catalog #62HIL02PEG, Cisbio) as described in the manufacturer manual.

Activation at 5000 activity (EC50) was calculated with the equation:

$$Y = Bottom + ( Top - Bottom ) / ( 1 + 10 \hat{} ( ( Log EC 50 - X ) * HillSlope ) )$$

X is log of compound concentration, Y is the IL-2 concentration, increasing as X increases.

Table 3 shows some EC50 values measured and/or calculated according to this biological example.

TABLE 3
Human PBMC IL-2 Assay (EC₅₀) (A: <500 nM;
B: 500 nM-1 uM (micromolar); C: >1 uM)

| Compound # | PBMC IL-2 assay EC50 |
|---|---|
| 5 | B |
| 6 | C |
| 7 | A |
| 17 | A |
| 19 | B |
| 48 | C |

### Biological Assay Example C: Jurkat IL-2 Assay

Jurkat cells (ATCC) were cultured in RPMI-1640 medium supplemented with 10% FBS. In a 96-well plate, 250,000 cells in 130 uL were added to each assay well. Ten microliter compound solution (5 uM to 2.3 nM, 8 points with 3-fold dilution) were added to cells in each well and incubated for 60 minutes at 37° C. Then 10 uL anti-CD3/CD28 antibody (Catalog #10991, Stemcell) were added and incubated overnight at 37° C. After the incubation, 16 uL supernatants were transferred into a new 384-well white assay plate for cytokine analysis. IL-2 was quantified using Human IL-2 kit (Catalog #62HIL02PEG, Cisbio) as described in the manufacturer manual.

Activation at 50% activity (EC50) was calculated with the equation:

$$Y = Bottom + ( Top - Bottom ) / ( 1 + 10 \hat{} ( ( Log EC 50 - X ) * HillSlope ) )$$

X is log of compound concentration, Y is the IL-2 concentration, increasing as X increases.

Table 4 shows some EC50 values measured and/or calculated according to this biological example.

TABLE 4
Jurkat IL-2 Assay (EC₅₀) (A: <500 nM; B:
500 nM-1 uM (micromolar); C: >1 uM)

| Compound # | Jurkat IL-2 assay EC50 |
|---|---|
| 21 | C |
| 45 | B |
| 50 | A |

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the ordinary skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the ordinarily skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A compound of Formula I, or a pharmaceutically acceptable salt thereof: 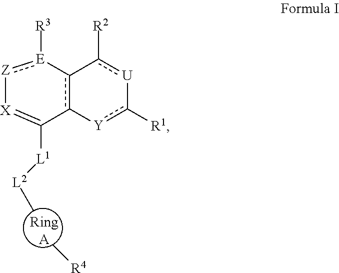
wherein:
R¹ is hydrogen, halogen (e.g., F, Cl, or Br), CN, OH, COOH, CONH₂, NH₂, R^{A}, OR^{A}, COOR^{A}, NH(R^{A}), N(R^{A})₂, CONH(R^{A}), CON(R^{A})₂, SR^{A}, SOR^{A}, SO₂R^{A}, or P(O)(R^{A})₂, wherein R^{A} at each occurrence is independently optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
R² is hydrogen, halogen (e.g., F, Cl, or Br), CN, OH, NH₂, R^{B}, OR^{B}, NH(R^{B}), or N(R^{B})₂, wherein R^{B} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, or optionally substituted 4-7 membered heterocyclyl;
R³ is hydrogen, optionally substituted C₁₋₄ alkyl, optionally substituted C₃₋₆ cycloalkyl, or optionally substituted 4-7 membered heterocyclyl (e.g., 4-7 membered saturated heterocyclyl);
E is N;
X is N or CR⁵, wherein R⁵ is hydrogen, halogen, CN, CONH₂, COOH, CONH(R^{C}), CON(R^{C})₂, OR^{C}, NO₂, or COOR^{C}, wherein R^{C} at each occurrence is independently an optionally substituted C₁₋₄ alkyl;
U is N or CR⁶, wherein R⁶ is hydrogen, halogen (e.g., F, Cl, or Br), CN, OH, NH₂, R^{D}, OR^{D}, NH(R^{D}), N(R^{D})₂, COOH, CONH₂, COOR^{D}, CONH(R^{D}), CON(R^{D})₂, SR^{D}, SOR^{D}, SO₂R^{D}, or P(O)(R^{D})₂, wherein R^{D} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
Y is N or CR⁷, wherein R⁷ is hydrogen, F, Cl, CN, optionally substituted C₁₋₄ alkyl, or optionally substituted C₁₋₄ heteroalkyl;
Z is C(O), C(═N—OH), C(═N—O—(C₁₋₄ alkyl)), C(═N—NH₂), C(═N—NH—(C₁₋₄ alkyl)), C(═N—N(C₁₋₄ alkyl)(C₁₋₄ alkyl)), SO, SO₂, S(O)(═NH), or S(O)(═N—(C₁₋₄ alkyl);
or Y and R¹ together are joined to form an optionally substituted ring selected from a 5 or 6 membered heteroaryl ring having 1-3 ring heteroatoms independently selected from O, S, and N, or a 4-7 membered carbocyclic or heterocyclic;
or U and R¹ together are joined to form an optionally substituted ring selected from a 5 or 6 membered heteroaryl having 1-3 ring heteroatoms independently selected from O, S, and N, or a 4-7 membered carbocyclic or heterocyclic;
or Z, E, and R³ together are joined to form an optionally substituted ring selected from a 5 or 6 membered heteroaryl having 1-3 ring heteroatoms independently selected from O, S, and N, or a 4-7 membered heterocycle;
or R² and R³ together are joined to form an optionally substituted 5-7 membered heterocycle;
or X and L¹ together are joined to form an optionally substituted fused ring;
L¹ is an optionally substituted 4-12 membered heterocyclylene having one or more rings and 1-4 ring heteroatoms each independently O, N, or S, or —N(R^{E})—, wherein R^{E} is optionally substituted C₁₋₄ alkyl or optionally substituted 3-7 membered ring;
L² is absent, O, NH, —N(R^{E2})—, C(O), SO₂, an optionally substituted C₁₋₄ alkylene, an optionally substituted C₂₋₄ alkenylene, an optionally substituted C₁₋₄ alkynylene, optionally substituted C₁₋₄ heteroalkylene, or an optionally substituted 3-7 membered ring, wherein R^{E2} is optionally substituted C₁₋₄ alkyl or optionally substituted 3-7 membered ring; and
Ring A and R⁴ together represent optionally substituted phenyl, optionally substituted 5 or 6 membered heteroaryl, or optionally substituted 8-12 membered ring having two or more rings;
provided that when X is N, Z is C(O), Y is N, U is CH, and L¹ is optionally substituted piperazine, then,
Ring A is an optionally substituted phenylene or heteroarylene; and R⁴ is SR^{F} SF₅, or optionally substituted 5-8 membered carbocyclic having two or more rings, wherein R^{F} is optionally substituted C₁₋₄ alkyl, such as CF₃, or optionally substituted C₃₋₆ cycloalkyl; or
Ring A and R⁴ together represent an optionally substituted 8-12 membered ring having two or more rings.

2. The compound of claim 1, or a pharmaceutically acceptable salt thereof, wherein Ring A is an optionally substituted phenylene; and R⁴ is SR^{F1}, SF₅, or optionally substituted 5-8 membered carbocyclic having two or more rings, such as a bridged bicyclic carbocyclic, wherein R^{F1} is optionally substituted C₁₋₄ alkyl, such as CF₃,
wherein, when substituted, the optionally substituted phenylene or 5-8 membered carbocyclic can be substituted with one or more (e.g., 1-3) substituents, for example, each substituent can be independently selected from halogen (e.g., F), OH, CN, C₁₋₄ alkyl or C₁₋₄ alkoxy.

3. The compound of claim 2, or a pharmaceutically acceptable salt thereof, wherein Ring A is an optionally substituted phenylene, such as unsubstituted phenylene, such as

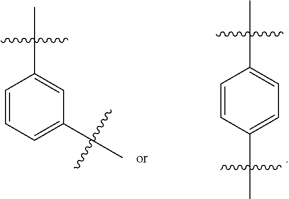 [SMILES: *c1cccc(*)c1 ; *c1ccc(*)cc1]

4. The compound of claim 1, or a pharmaceutically acceptable salt thereof, wherein Ring A is an optionally substituted 5 or 6-membered heteroarylene having 1-3 ring heteroatoms independently selected from O, N, and S; and R⁴ is SR^{F1}, SF₅, or optionally substituted 5-8 membered carbocyclic having two or more rings, such as a bridged bicyclic carbocyclic, wherein R^{F1} is optionally substituted C₁₋₄ alkyl, such as CF₃, wherein, when substituted, the optionally substituted heteroarylene or 5-8 membered carbocyclic can be substituted with one or more (e.g., 1-3) substituents, for example, each substituent can be independently selected from halogen (e.g., F), OH, CN, C₁₋₄ alkyl or C₁₋₄ alkoxy.

5. The compound of claim 4, or a pharmaceutically acceptable salt thereof, wherein Ring A is an optionally substituted pyridylene or pyrimidinylene, such as unsubstituted pyridylene or pyrimidinylene,

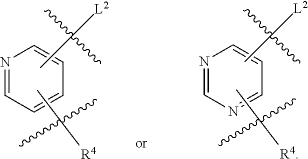

6. The compound of claim 4 or 5, or a pharmaceutically acceptable salt thereof, wherein Ring A is a pyridylene selected from the following (R⁴ is shown to show direction of attachment of the pyridylene):

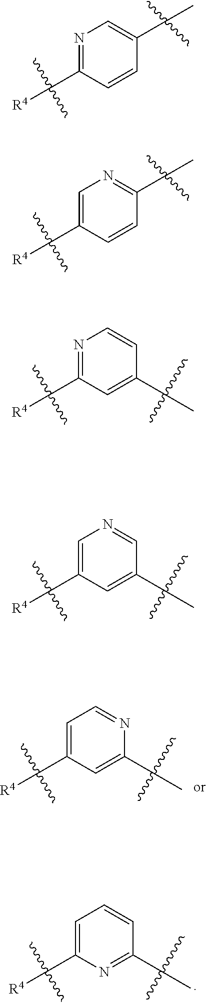

7. The compound of any of claims 1-6, or a pharmaceutically acceptable salt thereof, wherein R⁴ is SCF₃ or SF₅.

8. The compound of any of claims 1-6, or a pharmaceutically acceptable salt thereof, wherein R⁴ is

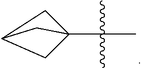 [SMILES: *C12CC(C1)C2]

9. The compound of claim 1, or a pharmaceutically acceptable salt thereof, wherein Ring A and R⁴ together represent a structure of formula S-1, S-2, S-3, or S-4 below: 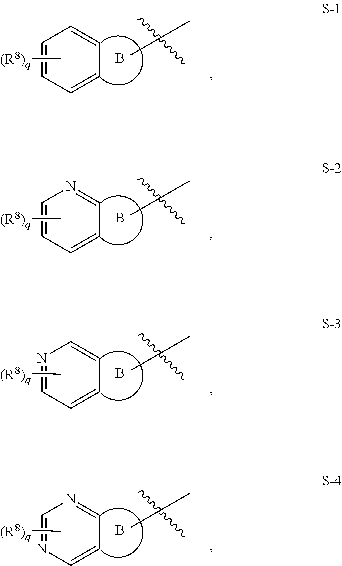
wherein:
Ring B is attached to L², wherein Ring B is a 5-7 membered ring containing 0, 1, or 2 ring heteroatoms, and is optionally substituted with 1-3 R^{G}, wherein R^{G} at each occurrence is independently halogen, OH, oxo, NH₂, NH(C₁₋₄ alkyl), N(C₁₋₄ alkyl)(C₁₋₄ alkyl), an optionally substituted C₁₋₄ alkyl, or optionally substituted C₁₋₄ alkoxy;
wherein the phenyl, pyridyl, or pyrimidyl in S-1, S-2, S-3, or S-4 is optionally substituted with q instance(s) of R⁸; wherein:
q is an integer ranging from 0-3 as valency permits, preferably, 1 or 2,
and R⁸ at each occurrence is independently halogen (e.g., F, Cl, or Br), CN, OH, NH₂, R^{H}, OR^{H}, NH(R^{H}), N(R^{H})₂, SR^{H}, SF₅, or optionally substituted 5-8 membered carbocyclic having two or more rings, wherein R^{H} at each occurrence is independently optionally substituted C₁₋₄ alkyl, optionally substituted C₂₋₄ alkenyl, optionally substituted C₂₋₄ alkynyl, optionally substituted C₃₋₆ cycloalkyl, or optionally substituted 4-7 membered heterocyclyl;
wherein, when substituted, the optionally substituted C₁₋₄ alkyl, C₁₋₄ alkoxy, 5-8 membered carbocyclic, C₂₋₄ alkenyl, C₂₋₄ alkynyl, C₃₋₆ cycloalkyl, or 4-7 membered heterocyclyl can be substituted with one or more (e.g., 1-3) substituents, for example, each substituent can be independently selected from halogen (e.g., F), OH, CN, C₁₋₄ alkyl or C₁₋₄ alkoxy.

10. The compound of claim 9, or a pharmaceutically acceptable salt thereof, wherein Ring B is a 5-7 membered ring containing no ring heteroatoms, which is optionally substituted with 1-3 RG, wherein R^{G1} at each occurrence is independently F, OH, C₁₋₄ alkyl optionally substituted with 1-3 F, or Cu₄ alkoxy optionally substituted with 1-3 F.

11. The compound of claim 9 or 10, or a pharmaceutically acceptable salt thereof, wherein R⁸ at each occurrence is independently R^{H1}, OR^{H1}, SR^{H1}, SF₅, or optionally substituted 5-8 membered carbocyclic having two or more rings, wherein R^{H1} is C₁₋₄ alkyl optionally substituted with 1-3 F, C₁₋₄ alkoxy optionally substituted with 1-3 F, or C₃₋₆ cycloalkyl optionally substituted with 1-3 substituents independently selected from F, OH, methyl, and methoxy, and wherein when substituted, the optionally substituted 5-8 membered carbocyclic is substituted with 1-3 substituents each independently selected from halogen (e.g., F), OH, CN, C₁₋₄ alkyl and C₁₋₄ alkoxy.

12. The compound of any of claims 9-11, or a pharmaceutically acceptable salt thereof, wherein q is 1.

13. The compound of any of claims 9-12, or a pharmaceutically acceptable salt thereof, wherein Ring A and R⁴ together represent a structure according to formula S-1-A, S-1-B, or S-1-C:

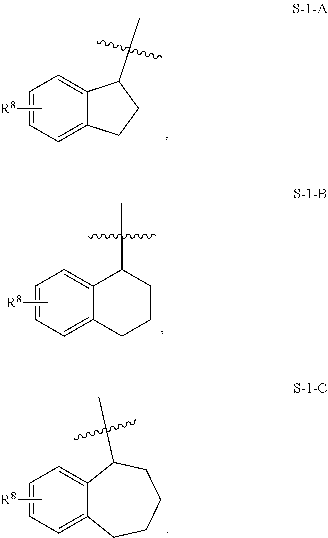

14. The compound of any of claims 9-13, or a pharmaceutically acceptable salt thereof, wherein R⁸ at each occurrence is independently a C₁₋₄ alkyl optionally substituted with 1-3 F (such as CH₃, CF₃, etc.), C₁₋₄ alkoxy optionally substituted with 1-3 F (such as OCH₃, OCF₃, etc.), SCF₃, SF₅, cyclopropyl, cyclobutyl, or

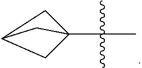 [SMILES: *C12CC(C1)C2]

15. The compound of claim 1, or a pharmaceutically acceptable salt thereof, characterized as having a structure according to Formula I-1, I-2, I-3, I-4, I-5, or I-6: 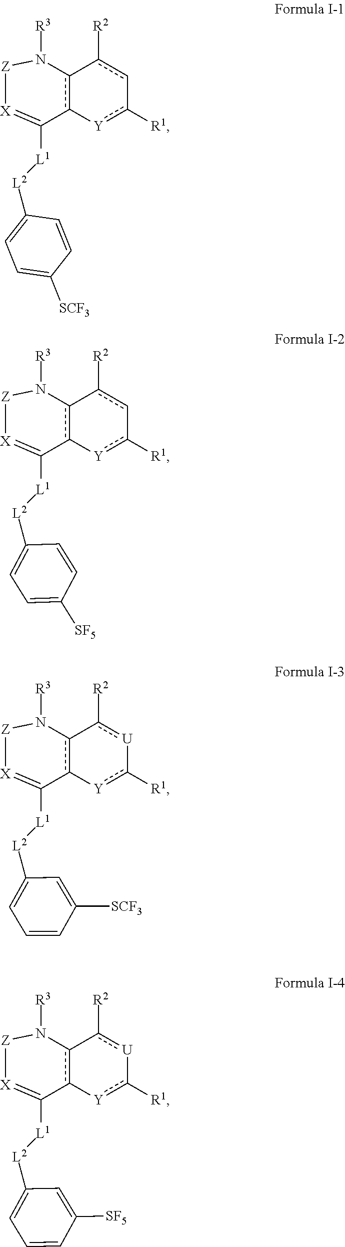 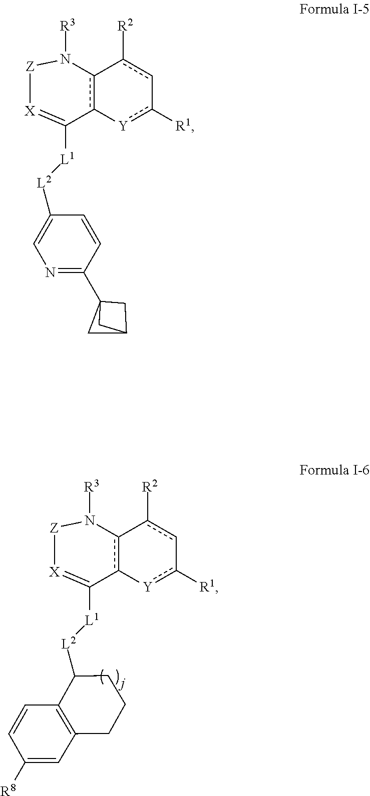
wherein:
j is 0, 1, or 2, and
R⁸ is halogen (e.g., F, Cl, or Br), CN, OH, NH₂, R^{H}, OR^{H}, NH(R^{H}), N(R^{H})₂, SR^{H}, SF₅, or optionally substituted 5-8 membered carbocyclic having two or more rings, wherein R^{H} at each occurrence is independently optionally substituted C₁₋₄ alkyl, optionally substituted C₂₋₄ alkenyl, optionally substituted C₂₋₄ alkynyl, optionally substituted C₃₋₆ cycloalkyl, or optionally substituted 4-7 membered heterocyclyl;
wherein, when substituted, the optionally substituted 5-8 membered carbocyclic, C₁₋₄ alkyl, C₂₋₄ alkenyl, C₂₋₄ alkynyl, C₃₋₆ cycloalkyl, or 4-7 membered heterocyclyl can be substituted with one or more (e.g., 1-3) substituents, for example, each substituent can be independently selected from halogen (e.g., F), OH, CN, C₁₋₄ alkyl or C₁₋₄ alkoxy.

16. The compound of claim 15 having formula I-6, or a pharmaceutically acceptable salt thereof, wherein R⁸ is R^{H1}, OR^{H1}, SR^{H1}, SF₅, or optionally substituted 5-8 membered carbocyclic having two or more rings, wherein R^{H1} is C₁₋₄ alkyl optionally substituted with 1-3 F, C₁₋₄ alkoxy optionally substituted with 1-3 F, or C₃₋₆ cycloalkyl optionally substituted with 1-3 substituents independently selected from F, OH, methyl, and methoxy, and wherein when substituted, the optionally substituted 5-8 membered carbocyclic is substituted with 1-3 substituents each independently selected from halogen (e.g., F), OH, CN, C₁₋₄ alkyl and C₁₋₄ alkoxy.

17. The compound of claim 16, or a pharmaceutically acceptable salt thereof, wherein R⁸ is C₁₋₄ alkyl optionally substituted with 1-3 F (such as CH₃, CF₃, etc.), C₁₋₄ alkoxy optionally substituted with 1-3 F (such as OCH₃, OCF₃, etc.), SCF₃, SF₅, cyclopropyl, cyclobutyl, or

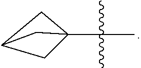 [SMILES: *C12CC(C1)C2]

18. The compound of any of claims 1-17, or a pharmaceutically acceptable salt thereof, wherein Y is N.

19. The compound of any of claims 1-17, or a pharmaceutically acceptable salt thereof, wherein Y is CH.

20. The compound of any of claims 1-17, or a pharmaceutically acceptable salt thereof, wherein Y and R¹ together form a 5 or 6 membered heteroaryl having 1-3 ring heteroatoms independently selected from O, S, and N.

21. The compound of claim 20, or a pharmaceutically acceptable salt thereof, wherein Y and R¹ together form a 5-membered heteroaryl having 1-3 ring nitrogen atoms.

22. The compound of claim 20 or 21, or a pharmaceutically acceptable salt thereof, characterized as having Formula I-A:

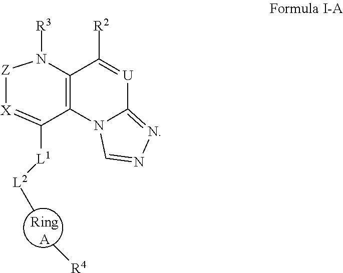

23. The compound of any of claims 1-22, or a pharmaceutically acceptable salt thereof, wherein U is N.

24. The compound of any of claims 1-22, or a pharmaceutically acceptable salt thereof, wherein U is CR⁶.

25. The compound of claim 24, or a pharmaceutically acceptable salt thereof, wherein R⁶ is hydrogen.

26. The compound of claim 24, or a pharmaceutically acceptable salt thereof, wherein R⁶ is halogen or OR^{D1}, wherein R^{D1} is optionally substituted C₁₋₄ alkyl, optionally substituted C₃₋₆ cycloalkyl, or optionally substituted 4-7 membered heterocyclyl having 1 or 2 ring heteroatoms independently selected from O, N, and S; wherein, when substituted, the optionally substituted C₁₋₄ alkyl, C₃₋₆ cycloalkyl, or 4-7 membered heterocyclyl can be substituted with one or more (e.g., 1-3) substituents, for example, each substituent can be independently selected from halogen (e.g., F), OH, CN, C₁₋₄ alkyl or C₁₋₄ alkoxy.

27. The compound of claim 24, or a pharmaceutically acceptable salt thereof, wherein R⁶ is C₁₋₄ alkoxy or

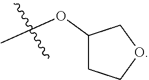 [SMILES: *OC1CCOC1]

28. The compound of any of claims 1-19, or a pharmaceutically acceptable salt thereof, wherein U and R¹ together form a 5 or 6 membered heteroaryl having 1-3 ring heteroatoms independently selected from O, S, and N, or a 5-7 membered heterocyclyl having 1 or 2 ring heteroatoms independently selected from O, S, and N.

29. The compound of any of claims 1-19 and 23-27, or a pharmaceutically acceptable salt thereof, wherein as applicable, R¹ is CN.

30. The compound of any of claims 1-19 and 23-27, or a pharmaceutically acceptable salt thereof, wherein as applicable, R¹ is halogen, e.g., F or Cl.

31. The compound of any of claims 1-19 and 23-27, or a pharmaceutically acceptable salt thereof, wherein as applicable, R¹ is C₂₋₃ alkynyl, such as

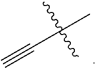 [SMILES: *C#C]

32. The compound of any of claims 1-19 and 23-27, or a pharmaceutically acceptable salt thereof, wherein as applicable, R¹ is optionally substituted 5 or 6 membered heteroaryl having 1-4 ring heteroatoms independently selected from O, S, and N.

33. The compound of any of claims 1-19 and 23-27, or a pharmaceutically acceptable salt thereof, wherein as applicable, R¹ is an optionally substituted pyrimidinyl or an optionally substituted thiazolyl, for example, R¹ is

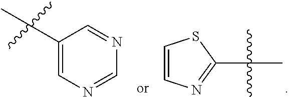 [SMILES: *c1cncnc1 ; *c1nccs1]

34. The compound of any of claims 1-19 and 23-27, or a pharmaceutically acceptable salt thereof, wherein as applicable, R¹ is SR^{A1}, wherein R^{A1} is independently optionally substituted C₁₋₄ alkyl, for example, R¹ is SCH₃.

35. The compound of any of claims 1-34, or a pharmaceutically acceptable salt thereof, wherein R² is hydrogen.

36. The compound of any of claims 1-35, or a pharmaceutically acceptable salt thereof, wherein R³ is an optionally substituted C₁₋₄ alkyl, such as CH₃, CD₃ etc.

37. The compound of any of claims 1-34, or a pharmaceutically acceptable salt thereof, wherein R² and R³ together are joined to form a 5-7 membered heterocycle having 0, 1, or 2 ring heteroatoms selected from O, N, and S, in addition to the nitrogen atom to which R³ is attached.

38. The compound of any of claims 1-34, or a pharmaceutically acceptable salt thereof, characterized as having Formula I-B:

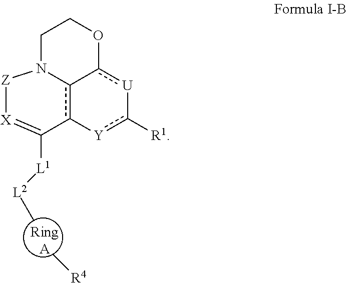

39. The compound of any of claims 1-38, or a pharmaceutically acceptable salt thereof, wherein Z is C(O).

40. The compound of any of claims 1-38, or a pharmaceutically acceptable salt thereof, wherein Z is S(O)₂.

41. The compound of any of claims 1-35, or a pharmaceutically acceptable salt thereof, wherein Z, E, and R³ together form a 5 or 6 membered heteroaryl having 1-3 ring heteroatoms independently selected from O, S, and N.

42. The compound of claim 41, or a pharmaceutically acceptable salt thereof, wherein Z, E, and R³ together form a 5-membered heteroaryl having 1-3 (preferably 2 or 3) ring nitrogen atoms.

43. The compound of claim 41, or a pharmaceutically acceptable salt thereof, characterized as having Formula I-C:

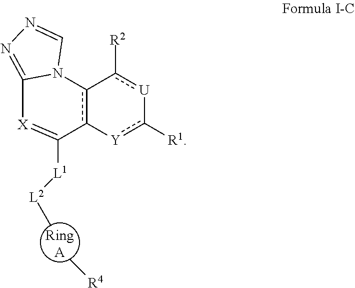

44. The compound of any of claims 1-43, or a pharmaceutically acceptable salt thereof, wherein X is N.

45. The compound of any of claims 1-43, or a pharmaceutically acceptable salt thereof, wherein X is CR, wherein R⁵ is CN.

46. The compound of any of claims 1-43, or a pharmaceutically acceptable salt thereof, wherein X is CR⁵, wherein R⁵ is CONH₂, COOH, CONH(R^{C1}), CON(R^{C1})₂, or COOR^{C1} wherein R¹ at each occurrence is independently a C₁₋₄ alkyl, such as methyl.

47. The compound of any of claims 1-43, or a pharmaceutically acceptable salt thereof, wherein X is CR⁵, wherein R⁵ is joined with L¹ to form an optionally substituted fused ring.

48. The compound of any of claims 1-47 as applicable, or a pharmaceutically acceptable salt thereof, characterized as having a formula according to Formula I-A-1, I-B-1, I-C-1, I-D, or I-E:

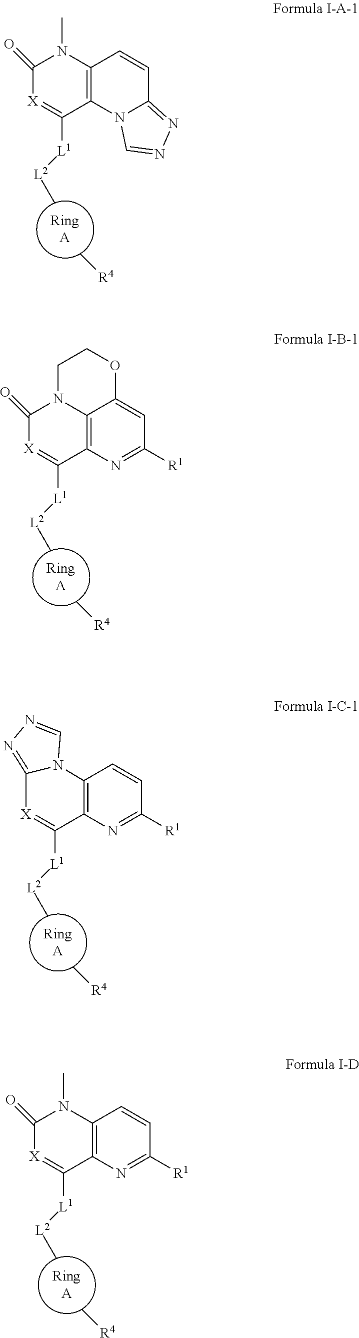

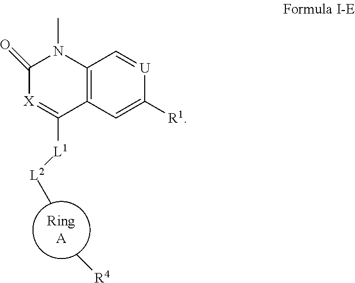

49. The compound of any of claims 1-48, or a pharmaceutically acceptable salt thereof, wherein L¹ is an optionally substituted 4-7 membered monocyclic heterocyclylene having 1 or 2 ring heteroatoms each independently O, N, or S.

50. The compound of any of claims 1-48, or a pharmaceutically acceptable salt thereof, wherein L¹ is 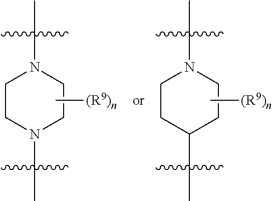
(either of the two attaching points of the piperidine, N or C, can be attached to L²),
wherein:
n is 0, 1, 2, 3, or 4, and
(1) R⁹ at each occurrence is independently R^{N}, COOR^{N}, COR^{N}, CONH(R^{N}), or CON(R^{N})₂, wherein R^{N} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, or optionally substituted 5 or 6 membered heteroaryl;
(2) two instances of R⁹ can be joined to form a double bond or a 3-5 membered ring, and any remaining instance(s) of R⁹ are as defined in (1); or
(3) when X is CR⁵, one instance of R⁹ and R⁵ can be joined to form a 5-7 membered ring and any remaining instance(s) of R⁹ are as defined in (1).

51. The compound of claim 50, or a pharmaceutically acceptable salt thereof, wherein R⁹ at each occurrence is independently CH₃, CH₂CH₃, CH₂CH₂CH₃, fluorine substituted C₁₋₃ alkyl (e.g., CF₂H), cyclopropyl, cyclobutyl, CH₂OH, CH₂OCH₃, CH₂OCH₂CH₃, CH₂NH₂, CH₂N₃, or CH₂NHC(O)OCH₃.

52. The compound of any of claims 1-48, or a pharmaceutically acceptable salt thereof, wherein L¹ is selected from the following (the bottom attaching point, N or C, is attached to L²):

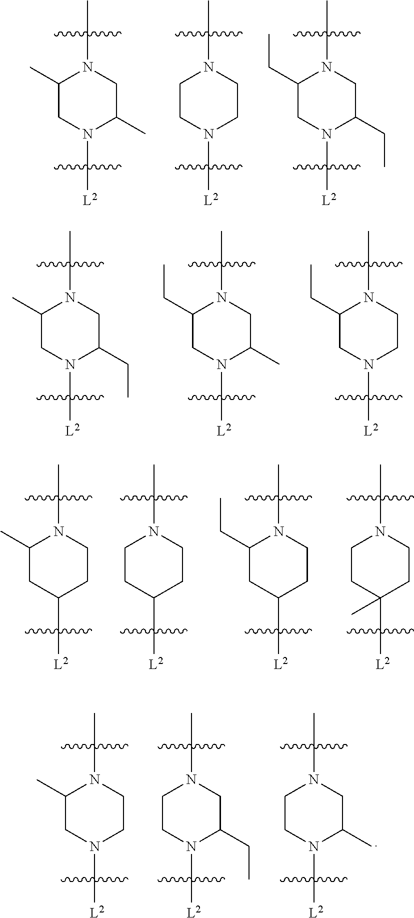

53. The compound of any of claims 1-48, or a pharmaceutically acceptable salt thereof, wherein L¹ is selected from the following (the bottom attaching point, N or C, is attached to L²):

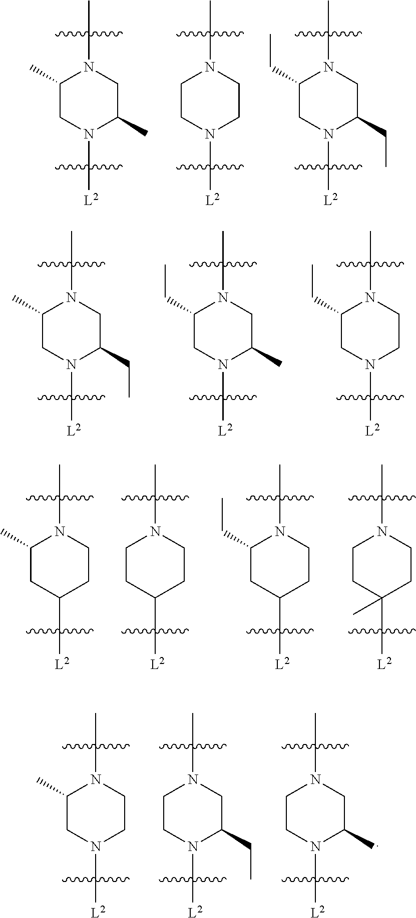

54. The compound of claim 50, or a pharmaceutically acceptable salt thereof, wherein X is CR⁵, and one instance of R⁹ is joined with R⁵, together with the intervening atoms, to form a 5-7 membered ring, and the other R⁹ group(s), if present, are as defined in claim 50 (1) or 51.

55. The compound of claim 54, or a pharmaceutically acceptable salt thereof, characterized as having Formula I-D-1 or I-D-2: 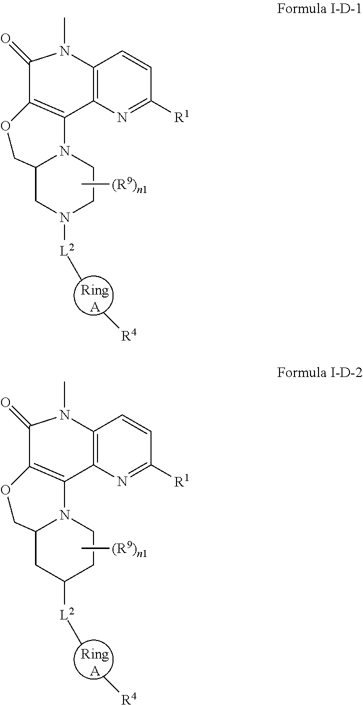
wherein n1 is 0, 1, 2, or 3.

56. The compound of any of claims 1-48, or a pharmaceutically acceptable salt thereof, wherein L¹ is an optionally substituted 7-12 membered heterocyclylene having two or more rings and 1-4 ring heteroatoms each independently O, N, or S, when substituted, the substituent(s) can be attached to any one or more of the two or more rings.

57. The compound of claim 56, or a pharmaceutically acceptable salt thereof, wherein L¹ is an optionally substituted 8-12 membered fused, spiro, or bridged bicyclic heterocyclylene having 1-4 ring heteroatoms each independent O, N, or S, wherein each ring of the bicyclic heterocyclylene can be saturated, partially unsaturated, or aromatic, and each ring of the bicyclic heterocyclylene can have 0, 1, 2, or 3 ring heteroatoms, provided that the bicyclic heterocyclylene as a whole is not fully aromatic and the total number of heteroatoms in the bicyclic heterocyclylene does not exceed 4.

58. The compound of claim 57, or a pharmaceutically acceptable salt thereof, wherein L¹ is an optionally substituted 8-11 membered fused bicyclic heterocyclylene having 1-3 ring heteroatoms each independent O, N, or S, wherein (1) one of the two fused rings is phenyl or 5 or 6 membered heteroaryl, and (2) the other of the two fused rings is a 5-7 membered heterocycle having one or two ring heteroatoms each independently O, N, or S, preferably, the 5-7 membered heterocycle has at least one ring nitrogen atom.

59. The compound of claim 57, or a pharmaceutically acceptable salt thereof, wherein L¹ is an optionally substituted 8-11 membered spiro bicyclic heterocyclylene having 1-4 ring heteroatoms each independent O, N, or S, wherein (1) one of the two spiro rings is a 5-7 membered heterocycle having one or two ring heteroatoms each independently O, N, or S, preferably, the 5-7 membered heterocycle has one ring nitrogen atom, and (2) the other of the two spiro rings is a 4-6 membered heterocycle having 1-3 ring heteroatoms each independently O, N, or S, for example, one of the two spiro rings is a pyrrolidine, piperidine, azepane ring and the other of the two spiro rings is a azetidine, pyrrolidine, pyrrolidinone, piperidinone, oxazoline, isoxazoline, thiazoline, isothiazoline, etc.

60. The compound of any of claims 1-48, or a pharmaceutically acceptable salt thereof, wherein L¹ is selected from the following (the bottom attaching point, N or C, is attached to L²): 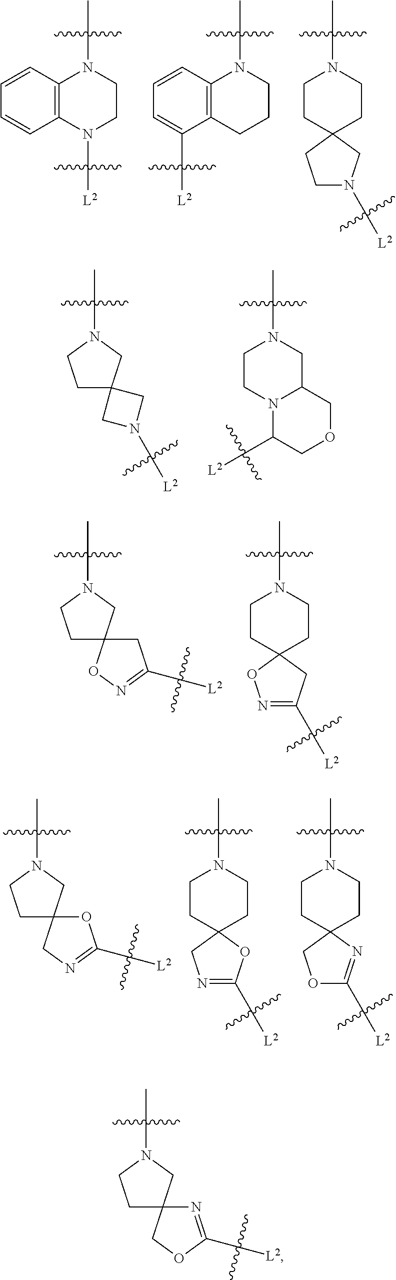
wherein in each of the bicyclic heterocyclylene, each of the two rings can be optionally substituted with 1-3 R¹⁰, wherein R¹⁰ at each occurrence is independently halogen, OH, NH₂, oxo (as applicable), R, OR, CN, NH(R^{J}), or N(R^{J})₂, wherein R^{J} at each occurrence is independently an optionally substituted C₁₋₄ alkyl, optionally substituted C₂₋₄ alkenyl, optionally substituted C₂₋₄ alkynyl, or optionally substituted 3-4 membered ring.

61. The compound of any of claims 1-48, or a pharmaceutically acceptable salt thereof, wherein L¹ is selected from the following (the bottom attaching point, N or C, is attached to L²): 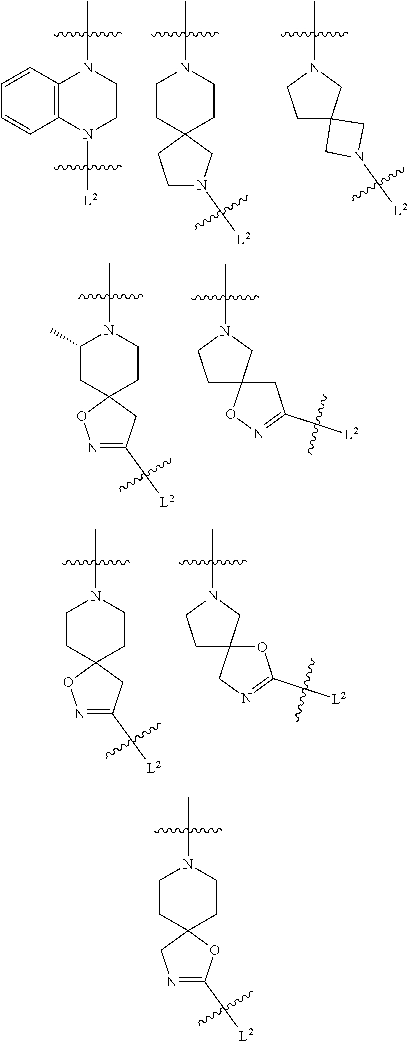
or L¹ is selected from L² or

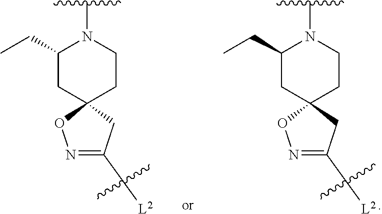

62. The compound of any of claims 1-48, or a pharmaceutically acceptable salt thereof, wherein L¹ is —N(C₁₋₄ alkyl)-, such as —N(CH₃)—.

63. The compound of any of claims 1-62, or a pharmaceutically acceptable salt thereof, wherein L² is absent.

64. The compound of any of claims 1-62, or a pharmaceutically acceptable salt thereof, wherein L² is O or C(O).

65. The compound of any of claims 1-62, or a pharmaceutically acceptable salt thereof, wherein L² is an optionally substituted C₁₋₄ alkylene, wherein, when substituted, the C₁₋₄ alkylene is substituted with 1-3 R¹¹, wherein R¹¹ at each occurrence is independently selected from halogen (e.g., F, Cl, or Br), CN, OH, NH₂, COOH, CONH₂, SO₂NH₂, COR^{K}, COOR^{K}, CONH(R^{K}), CON(R^{K})₂, SO₂R^{K}, SO₂NH(R^{K}), SO₂N(R^{K})₂, R^{K}, OR^{K}, NH(R^{K}), N(R^{K})₂, SR^{K}, SOR^{K}, SO₂R^{K}, or P(O)(R^{K})₂, wherein R^{K} at each occurrence is independently optionally substituted C₁₋₄ alkyl, optionally substituted C₂₋₄ alkenyl, optionally substituted C₂₋₄ alkynyl, optionally substituted C₁₋₄ heteroalkyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl.

66. The compound of any of claims 1-62, or a pharmaceutically acceptable salt thereof, wherein L² is an unsubstituted C₁₋₄ alkylene selected from: CH₂, or

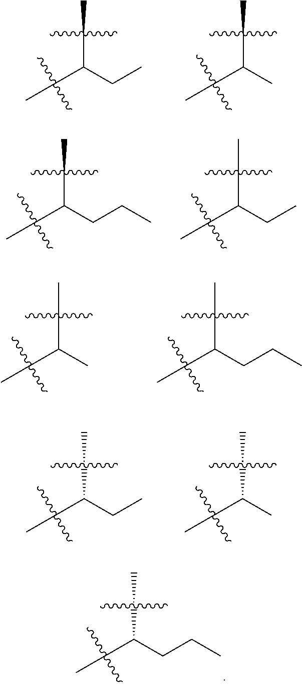

67. The compound of any of claims 1-62, or a pharmaceutically acceptable salt thereof, wherein L² is an R¹²-substituted C₁₋₄ alkylene selected from the following: 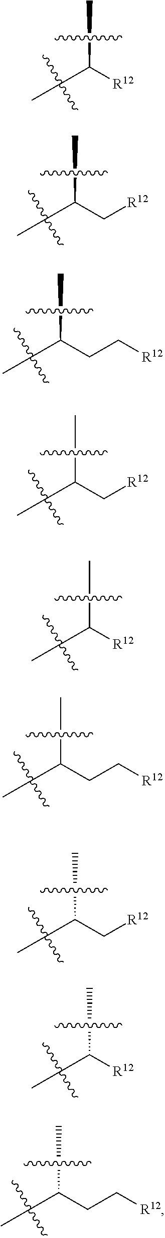
wherein:
R¹² is selected from halogen (e.g., F, Cl, or Br), CN, OH, NH₂, COOH, CONH₂, SO₂NH₂, COR^{K}, COOR^{K}, CONH(R^{K}), CON(R^{K})₂, SO₂R^{K}, SO₂NH(R^{K}), SO₂N(R^{K})₂, R^{K}, OR^{K}, NH(R^{K}), N(R^{K})₂, SR^{K}, SOR^{K}, SO₂R^{K}, or P(O)(R^{K})₂, wherein R^{K} at each occurrence is independently optionally substituted C₁₋₄ alkyl, optionally substituted C₂₋₄ alkenyl, optionally substituted C₂₋₄ alkynyl, optionally substituted C₁₋₄ heteroalkyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl,
wherein, when substituted, the optionally substituted C₁₋₄ alkyl, C₂₋₄ alkenyl, C₂₋₄ alkynyl, C₁₋₄ heteroalkyl, C₃₋₆ cycloalkyl, phenyl, 5 or 6 membered heteroaryl, or 4-7 membered heterocyclyl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

68. The compound of claim 67, or a pharmaceutically acceptable salt thereof, wherein R¹² is CN, OH, COOH, CONH₂, methoxy, ethoxy, cyclopropyl, cyclobutyl, optionally substituted phenyl, or optionally substituted 5 or 6 membered heteroaryl having 1-3 ring heteroatoms independently selected from N, O, and S, wherein, when substituted, the optionally substituted phenyl or 5 or 6 membered heteroaryl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

69. The compound of any of claims 1-62, or a pharmaceutically acceptable salt thereof, wherein L² is an optionally substituted 5 or 6 membered heteroarylene, for example,

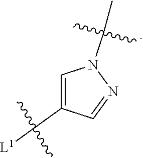

70. The compound of any of claims 1-62, or a pharmaceutically acceptable salt thereof, wherein L² is an optionally substituted phenylene, e.g.,

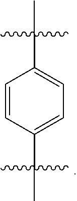 [SMILES: *c1ccc(*)cc1]

71. The compound of any of claims 1-48, or a pharmaceutically acceptable salt thereof, characterized as having a structure according to Formula I-L-1, I-L-2, I-L-3, I-L-4, I-L-5, I-L-6, I-L-7, I-L-8, I-L-9, I-L-10, or I-L-11: 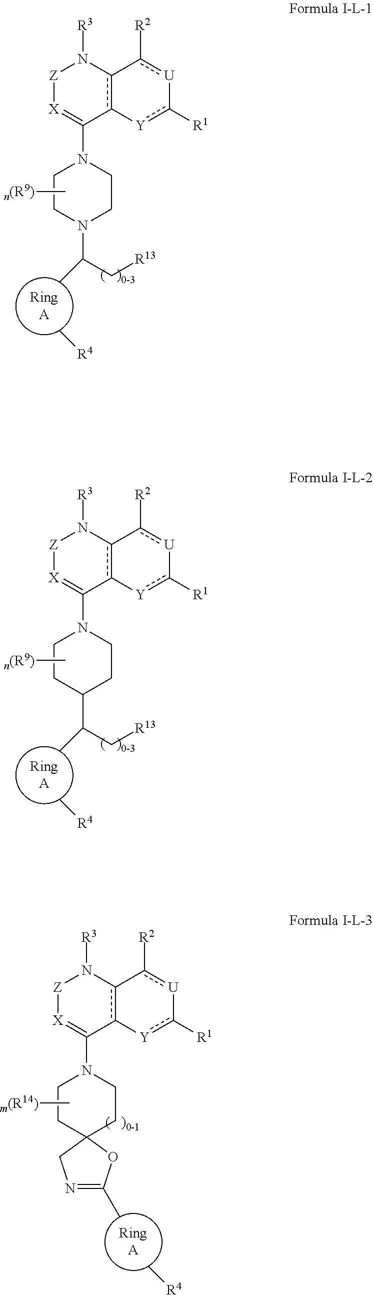 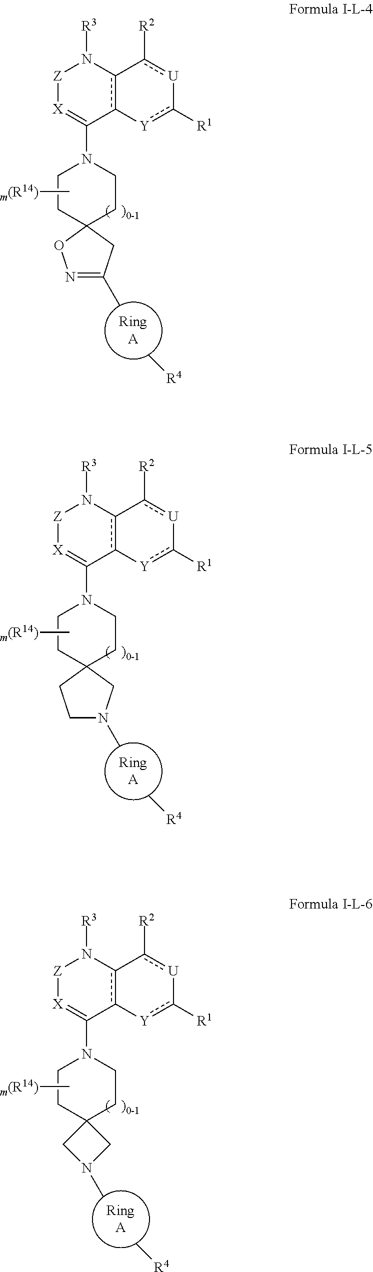 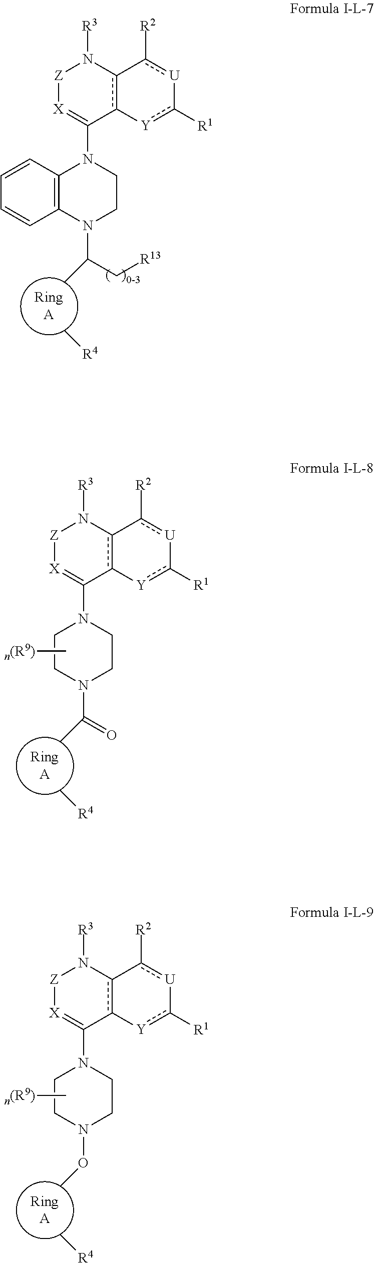 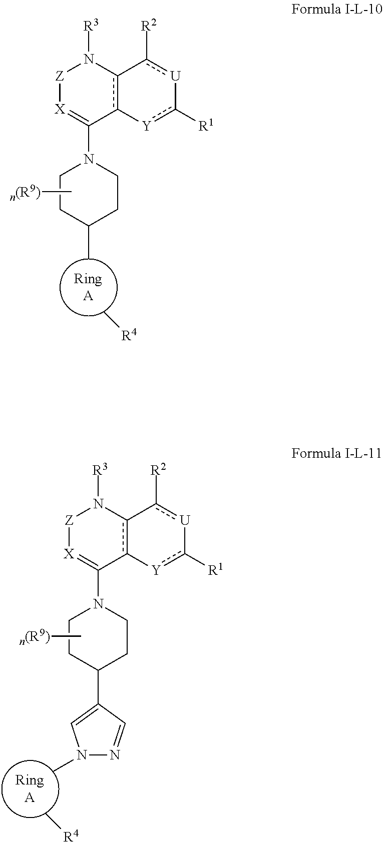
wherein:
X, Y, U, Z, R¹, R², R³, R⁴, and Ring A are as defined above in any of claims 1-48,
R⁹ at each occurrence is independently CH₃, CH₂CH₃, CH₂CH₂CH₃, fluorine substituted C₁₋₃ alkyl (e.g., CF₂H), cyclopropyl, cyclobutyl, CH₂OH, CH₂OCH₃, CH₂OCH₂CH₃, CH₂NH₂, CH₂N₃, or CH₂NHC(O)OCH₃;
n is 0, 1, 2, or 3;
R¹³ is selected from hydrogen, halogen (e.g., F, Cl, or Br), CN, OH, NH₂, COOH, CONH₂, SO₂NH₂, COR^{K}, COOR^{K}, CONH(R^{K}), CON(R^{K})₂, SO₂R^{K}, SO₂NH(R^{K}), SO₂N(R^{K})₂, R^{K}, OR^{K}, NH(R^{K}), N(R^{K})₂, SR^{K}, SOR^{K}, SO₂R^{K}, or P(O)(R^{K})₂, wherein R^{K} at each occurrence is independently optionally substituted C₁₋₄ alkyl, optionally substituted C₂₋₄ alkenyl, optionally substituted C₂₋₄ alkynyl, optionally substituted C₁₋₄ heteroalkyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl;
or one instance of R⁹ and R¹³ together with the intervening atom(s) form an optionally substituted 3-7 membered ring, and the other instance(s) of R⁹ at each occurrence is independently CH₃, CH₂CH₃, CH₂CH₂CH₃, CH₂OH, CH₂OCH₃, CH₂OCH₂CH₃, CH₂NH₂, CH₂N₃, or CH₂NHC(O)OCH₃;
R¹⁴ at each occurrence is independently an optionally substituted C₁₋₄ alkyl; and
m is 0, 1, or 2;
wherein, when substituted, the optionally substituted C₁₋₄ alkyl, C₂₋₄ alkenyl, C₂₋₄ alkynyl, C₁₋₄ heteroalkyl, C₃₋₆ cycloalkyl, phenyl, 5 or 6 membered heteroaryl, 4-7 membered heterocyclyl, or 3-7 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

72. The compound of claim 71, or a pharmaceutically acceptable salt thereof, wherein R¹³ is hydrogen, CN, OH, COOH, CONH₂, methoxy, ethoxy, cyclopropyl, cyclobutyl, optionally substituted phenyl, or optionally substituted 5 or 6 membered heteroaryl having 1-3 ring heteroatoms independently selected from N, O, and S, wherein, when substituted, the optionally substituted phenyl or 5 or 6 membered heteroaryl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

73. A compound of Formula II-1, II-2, II-3, II-4, II-5, II-6, or II-7, or a pharmaceutically acceptable salt thereof: 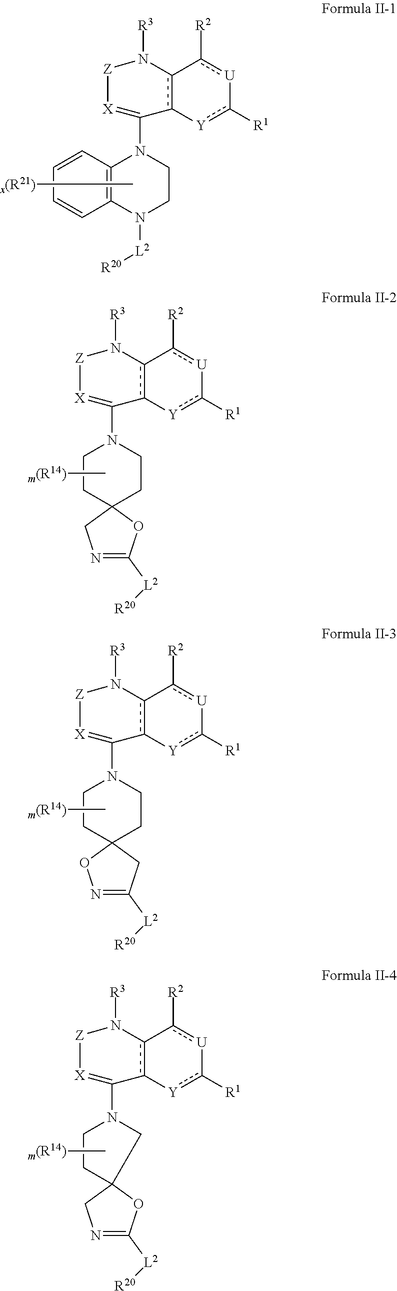 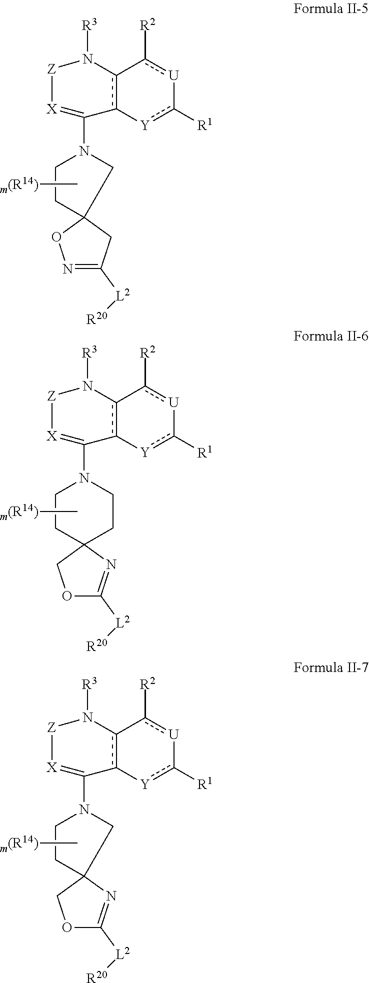
wherein:
R¹ is hydrogen, halogen (e.g., F, Cl, or Br), CN, OH, COOH, CONH₂, NH₂, R^{A}, OR^{A}, NH(R^{A}), N(R^{A})₂, COOR^{A}, CONH(R^{A}), CON(R^{A})₂, SR^{A}, SOR^{A}, SO₂R^{A}, or P(O)(R^{A})₂, wherein R^{A} at each occurrence is independently optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
R² is hydrogen, halogen (e.g., F, Cl, or Br), CN, OH, NH₂, R^{B}, OR^{B}, NH(R^{B}), or N(R^{B})₂, wherein R^{B} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, or optionally substituted 4-7 membered heterocyclyl;
R³ is hydrogen, optionally substituted C₁₋₄ alkyl, optionally substituted C₃₋₆ cycloalkyl, or optionally substituted 4-7 membered heterocyclyl (such as a saturated 4-7 membered heterocyclyl);
X is N or CR⁵, wherein R⁵ is hydrogen, halogen, CN, CONH₂, COOH, CONH(R^{C}), CON(R^{C})₂, OR^{C}, NO₂, or COOR^{C}, wherein R^{C} at each occurrence is independently an optionally substituted C₁₋₄ alkyl;
U is N or CR⁶, wherein R⁶ is hydrogen, halogen (e.g., F, Cl, or Br), CN, OH, NH₂, R^{D}, OR^{D}, NH(R^{D}), N(R^{D})₂, COOH, CONH₂, COOR^{D}, CONH(R^{D}), CON(R^{D})₂, SR^{D}, SOR^{D}, SO₂R^{D}, or P(O)(R^{D})₂, wherein R^{D} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
Y is N or CR⁷, wherein R⁷ is hydrogen, F, Cl, CN, optionally substituted C₁₋₄ alkyl, or optionally substituted C₁₋₄ heteroalkyl;
Z is C(O), C(═N—OH), C(═N—O—(C₁₋₄ alkyl)), C(═N—NH₂), C(═N—NH—(C₁₋₄ alkyl)), C(═N—N(C₁₋₄ alkyl)(C₁₋₄ alkyl)), SO, SO₂, S(O)(═NH), or S(O)(═N—(C₁₋₄ alkyl);
or Y and R¹ together are joined to form an optionally substituted ring selected from a 5 or 6 membered heteroaryl having 1-3 ring heteroatoms independently selected from O, S, and N, or a 4-7 membered carbocyclic or heterocyclic;
or U and R¹ together are joined to form an optionally substituted ring selected from a 5 or 6 membered heteroaryl having 1-3 ring heteroatoms independently selected from O, S, and N, or a 4-7 membered carbocyclic or heterocyclic;
or Z and R³ together are joined to form an optionally substituted ring selected from a 5 or 6 membered heteroaryl having 1-3 ring heteroatoms independently selected from O, S, and N, or a 4-7 membered heterocycle;
or R² and R³ together are joined to form an optionally substituted ring selected from a 5-7 membered heterocycle;
L² is absent, O, NH, —N(R^{E2})—, C(O), SO₂, an optionally substituted C₁₋₄ alkylene, an optionally substituted C₂₋₄ alkenylene, an optionally substituted C₁₋₄ alkynylene, optionally substituted C₁₋₄ heteroalkylene, or an optionally substituted 3-7 membered ring, wherein R^{E2} is optionally substituted C₁₋₄ alkyl or optionally substituted 3-7 membered ring;
R²⁰ is an optionally substituted phenyl, optionally substituted 5 or 6 membered heteroaryl, or optionally substituted 8-12 membered ring having two or more rings;
R¹⁴ at each occurrence is independently an optionally substituted C₁₋₄ alkyl;
m is 0, 1, or 2;
R²¹ at each occurrence is independently halogen (e.g., F, Cl, or Br), CN, OH, R^{L} OR^{L}, wherein R^{L} at each occurrence is independently an optionally substituted C₁₋₄ alkyl, optionally substituted C₂₋₄ alkenyl, optionally substituted C₂₋₄ alkynyl, or optionally substituted C₃₋₆ cycloalkyl; and
x is 0, 1, 2, or 3.

74. The compound of claim 73, or a pharmaceutically acceptable salt thereof, wherein Y is N.

75. The compound of claim 73, or a pharmaceutically acceptable salt thereof, wherein Y is CH.

76. The compound of claim 73, or a pharmaceutically acceptable salt thereof, wherein Y and R¹ together form a 5 or 6 membered heteroaryl having 1-3 ring heteroatoms independently selected from O, S, and N, such as a 5-membered heteroaryl having 1-3 ring nitrogen atoms, preferably,

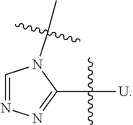

77. The compound of any of claims 73-76, or a pharmaceutically acceptable salt thereof, wherein U is N.

78. The compound of any of claims 73-76, or a pharmaceutically acceptable salt thereof, wherein U is CR⁶.

79. The compound of claim 78, or a pharmaceutically acceptable salt thereof, wherein R⁶ is hydrogen.

80. The compound of claim 78, or a pharmaceutically acceptable salt thereof, wherein R⁶ is C₁₋₄ alkoxy or

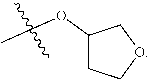 [SMILES: *OC1CCOC1]

81. The compound of any of claims 73-75 and 77-80, or a pharmaceutically acceptable salt thereof, wherein as applicable, R¹ is CN.

82. The compound of any of claims 73-75 and 77-80, or a pharmaceutically acceptable salt thereof, wherein as applicable, (i) R¹ is halogen, e.g., F or Cl; (ii) R¹ is 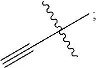 [SMILES: *C#C]
(iii) R¹ is an optionally substituted 5 or 6 membered heteroaryl having 1-4 ring heteroatoms independently selected from O, S, and N, such as an optionally substituted pyrimidinyl or an optionally substituted thiazolyl, for example, R¹ is 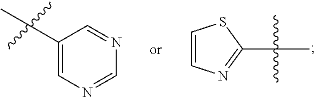 [SMILES: *c1cncnc1 ; *c1nccs1]
or (iv) R¹ is SR^{A1}, wherein R^{A1} is independently optionally substituted C₁₋₄ alkyl, for example, R¹ is SCH₃.

83. The compound of any of claims 73-82, or a pharmaceutically acceptable salt thereof, wherein R² is hydrogen.

84. The compound of any of claims 73-83, or a pharmaceutically acceptable salt thereof, wherein R³ is an optionally substituted C₁₋₄ alkyl, such as CH₃, CD₃ etc.

85. The compound of any of claims 73-82, or a pharmaceutically acceptable salt thereof, wherein R² and R³ together are joined to form a 5-7 membered heterocycle having 0, 1, or 2 ring heteroatoms selected from O, N, and S, in addition to the nitrogen atom to which R³ is attached, such as

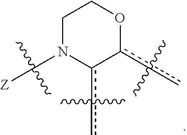

86. The compound of any of claims 73-85, or a pharmaceutically acceptable salt thereof, wherein Z is C(O).

87. The compound of any of claims 73-85, or a pharmaceutically acceptable salt thereof, wherein Z is S(O)₂.

88. The compound of any of claims 73-85, or a pharmaceutically acceptable salt thereof, wherein Z and R³ together form a 5 or 6 membered heteroaryl having 1-3 ring heteroatoms independently selected from O, S, and N such as a 5-membered heteroaryl having 1-3 (preferably 2 or 3) ring nitrogen atoms, preferably,

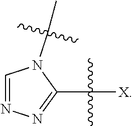

89. The compound of any of claims 73-88, or a pharmaceutically acceptable salt thereof, wherein X is N.

90. The compound of any of claims 73-88, or a pharmaceutically acceptable salt thereof, wherein X is CR⁵, wherein R⁵ is CN.

91. The compound of any of claims 73-88, or a pharmaceutically acceptable salt thereof, wherein X is CR⁵, wherein R⁵ is CONH₂, COOH, CONH(R^{C1}), CON(R^{C1})₂, or COOR^{C1} wherein R¹ at each occurrence is independently a C₁₋₄ alkyl, such as methyl.

92. The compound of any of claims 73-91, or a pharmaceutically acceptable salt thereof, wherein x in Formula II-1 is 0.

93. The compound of any of claims 73-91, or a pharmaceutically acceptable salt thereof, wherein m in Formula II-2, II-3, II-4, II-5, II-6, or II-7 is 0.

94. The compound of any of claims 73-91, or a pharmaceutically acceptable salt thereof, wherein m in Formula II-2, II-3, II-4, II-5, II-6, or II-7 is 1, and R¹⁴ is an optionally substituted C₁₋₄ alkyl, such as methyl, ethyl, etc.

95. The compound of any of claims 73-94, or a pharmaceutically acceptable salt thereof, wherein L² in Formula II-2, II-3, II-4, II-5, II-6, or II-7 is absent.

96. The compound of any of claims 73-94, or a pharmaceutically acceptable salt thereof, wherein L² in Formula II-1 is an optionally substituted C₁₋₄ alkylene selected from: 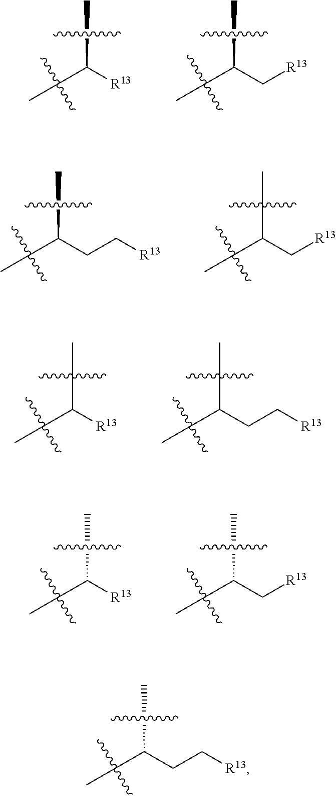
wherein:
R¹³ is selected from hydrogen, halogen (e.g., F, Cl, or Br), CN, OH, NH₂, COOH, CONH₂, SO₂NH₂, COR^{K}, COOR^{K}, CONH(R^{K}), CON(R^{K})₂, SO₂R^{K}, SO₂NH(R^{K}), SO₂N(R^{K})₂, R^{K}, OR^{K}, NH(R^{K}), N(R^{K})₂, SR^{K}, SOR^{K}, SO₂R^{K}, or P(O)(R^{K})₂, wherein R^{K} at each occurrence is independently optionally substituted C₁₋₄ alkyl, optionally substituted C₂₋₄ alkenyl, optionally substituted C₂₋₄ alkynyl, optionally substituted C₁₋₄ heteroalkyl, optionally substituted C₃₋₆ cycloalkyl, optionally substituted phenyl, optionally substituted 5 or 6 membered heteroaryl having 1-4 ring heteroatoms independently selected from 0, S, and N, or optionally substituted 4-7 membered heterocyclyl, wherein, when substituted, the optionally substituted C₁₋₄ alkyl, C₂₋₄ alkenyl, C₂₋₄ alkynyl, C₁₋₄ heteroalkyl, C₃₋₆ cycloalkyl, phenyl, 5 or 6 membered heteroaryl, or 4-7 membered heterocyclyl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

97. The compound of claim 96, or a pharmaceutically acceptable salt thereof, wherein R¹³ is hydrogen.

98. The compound of claim 96, or a pharmaceutically acceptable salt thereof, wherein R¹³ is CN, OH, COOH, CONH₂, methoxy, ethoxy, cyclopropyl, cyclobutyl, optionally substituted phenyl, or optionally substituted 5 or 6 membered heteroaryl having 1-3 ring heteroatoms independently selected from N, O, and S, wherein, when substituted, the optionally substituted phenyl or 5 or 6 membered heteroaryl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

99. The compound of any of claims 73-94, or a pharmaceutically acceptable salt thereof, wherein L² is 0 or C(O).

100. The compound of any of claims 73-99, or a pharmaceutically acceptable salt thereof, wherein R²⁰ is optionally substituted phenyl, such as a phenyl which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from 0, S, and N, or optionally substituted 4-7 membered heterocyclyl.

101. The compound of claim 100, or a pharmaceutically acceptable salt thereof, wherein R²⁰ is a phenyl which is substituted with 1-3 (e.g., 1 or 2) R²², wherein R²² at each occurrence is independently halogen (e.g., F, Cl, or Br), CN, OH, R^{M1}, OR^{M1}, SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

102. The compound of claim 100, or a pharmaceutically acceptable salt thereof, wherein R²⁰ is a phenyl which is substituted with 1-3 (e.g., 1 or 2) R²², wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

103. The compound of any of claims 73-99, or a pharmaceutically acceptable salt thereof, wherein R²⁰ is optionally substituted 5 or 6-membered heteroaryl, such as a pyridyl 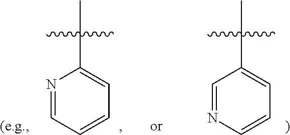 [SMILES: *c1ccccn1 ; *c1cccnc1]
which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl.

104. The compound of claim 103, or a pharmaceutically acceptable salt thereof, wherein R²⁰ is a 5-membered heteroaryl 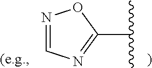 [SMILES: *c1ncno1]
or pyridyl 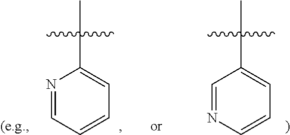 [SMILES: *c1ccccn1 ; *c1cccnc1]
which is substituted with 1-3 (e.g., 1 or 2) R²², as valency permits, wherein R²² at each occurrence is independently halogen (e.g., F, Cl, or Br), CN, OH, R^{M1}, OR^{M1}, SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

105. The compound of claim 103, or a pharmaceutically acceptable salt thereof, wherein R²⁰ is a 5-membered heteroaryl 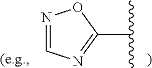 [SMILES: *c1ncno1]
or pyridyl 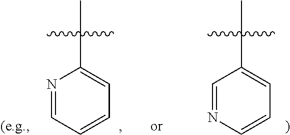 [SMILES: *c1ccccn1 ; *c1cccnc1]
which is substituted with 1-3 (e.g., 1 or 2) R²², as valency permits, wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

106. The compound of any of claims 73-99, or a pharmaceutically acceptable salt thereof, wherein R²⁰ has a structure according to S-1-A, S-1-B, or S-1-C: 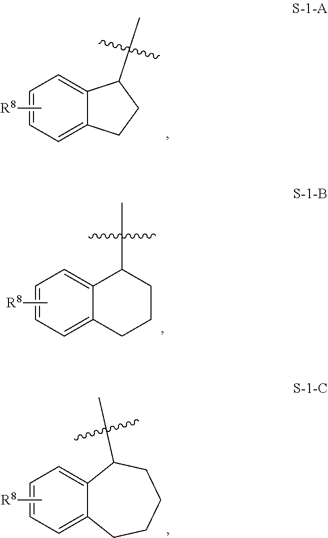
wherein R⁸ is C₁₋₄ alkyl optionally substituted with 1-3 F (such as CH₃, CF₃, etc.), C₁₋₄ alkoxy optionally substituted with 1-3 F (such as OCH₃, OCF₃, etc.), SCF₃, SF₅, cyclopropyl, cyclobutyl, or

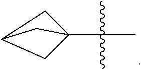 [SMILES: *C12CC(C1)C2]

107. A compound of Formula III-1, III-2, III-3, III-4, III-5, III-6, III-7, III-8, or III-9, or a pharmaceutically acceptable salt thereof: 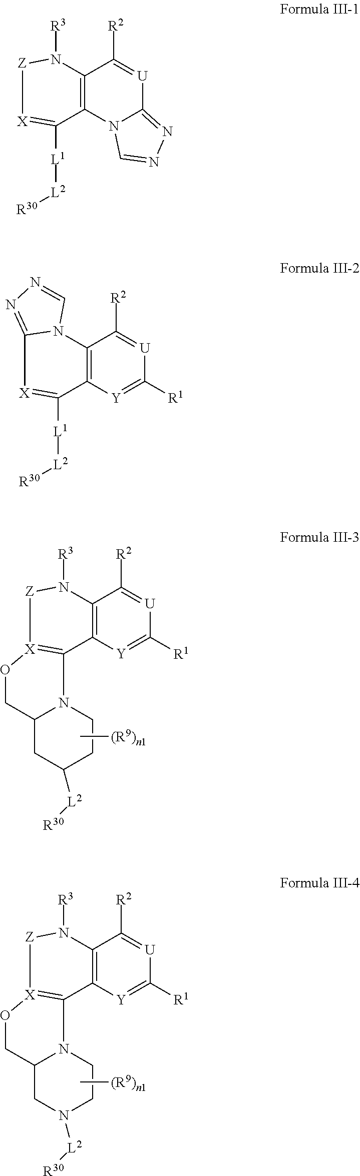 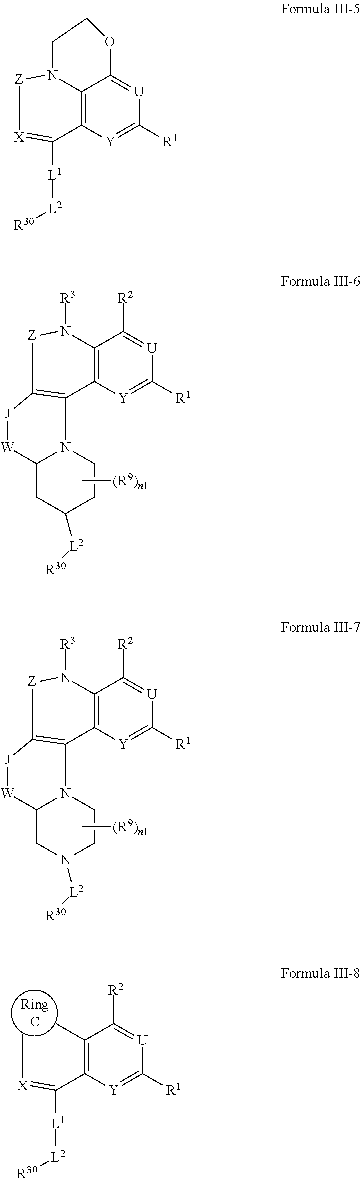 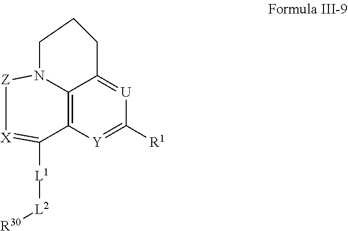
wherein:
R¹ is hydrogen, halogen (e.g., F, Cl, or Br), CN, OH, NH₂, COOH, CONH₂, R^{A}, OR^{A}, NH(R^{A}), N(R^{A})₂, COOR^{A}, CONH(R^{A}), CON(R^{A})₂, SR^{A}, SOR^{A}, SO₂R^{A}, or P(O)(R^{A})₂, wherein R^{A} at each occurrence is independently optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
R² is hydrogen, halogen (e.g., F, Cl, or Br), CN, OH, NH₂, R^{B}, OR^{B}, NH(R^{B}), or N(R^{B})₂, wherein R^{B} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, or optionally substituted 4-7 membered heterocyclyl;
R³ is hydrogen, optionally substituted C₁₋₄ alkyl, optionally substituted C₃₋₆ cycloalkyl, or optionally substituted 4-7 membered heterocyclyl (such as a saturated 4-7 membered heterocyclyl);
J is O, CO, SO₂, NR¹⁵ or optionally substituted methylene;
W is absent or (W¹)ₚ, wherein p is 1, 2, or 3, wherein W¹ at each occurrence is independently, C(O), NR¹, SO₂, O, or optionally substituted methylene, provided that at most one instance of W¹ is C(O), NR¹⁵, SO₂, or O;
wherein R¹⁵ at each occurrence is independently hydrogen, optionally substituted C₁₋₆ alkyl, optionally substituted C₂₋₆ alkenyl, optionally substituted C₂₋₆ alkynyl, optionally substituted 3-6 membered ring, or nitrogen protecting group;
X is N or CR⁵, wherein R⁵ is hydrogen, halogen, CN, CONH₂, COOH, CONH(R^{C}), CON(R^{C})₂, OR^{C}, or COOR^{C}, wherein R^{C} at each occurrence is independently an optionally substituted C₁₋₄ alkyl;
U is N or CR⁶, wherein R⁶ is hydrogen, halogen (e.g., F, Cl, or Br), CN, OH, NH₂, R^{D}, OR^{D}, NH(R^{D}), N(R^{D})₂, COOH, CONH₂, COOR^{D}, CONH(R^{D}), CON(R^{D})₂, SR^{D}, SOR^{D}, SO₂R^{D}, or P(O)(R^{D})₂, wherein R^{D} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
Y is N or CR⁷, wherein R⁷ is hydrogen, F, Cl, CN, optionally substituted C₁₋₄ alkyl, or optionally substituted C₁₋₄ heteroalkyl;
Z is C(O), C(═N—OH), C(═N—O—(C₁₋₄ alkyl)), C(═N—NH₂), C(═N—NH—(C₁₋₄ alkyl)), C(═N—N(C₁₋₄ alkyl)(C₁₋₄ alkyl)), SO, SO₂, S(O)(═NH), or S(O)(═N—(C₁₋₄ alkyl);
Ring C is a 5 or 6 membered ring, preferably, a 5 or 6 membered heteroaryl having 2-3 ring heteroatoms, such as a 5 membered heteroaryl having 2-3 ring nitrogen atoms, for example, imidazole or triazole ring, wherein Ring C shares two ring atoms with the adjacent ring in Formula III-8;
L¹ is an optionally substituted 4-12 membered heterocyclylene having one or more rings and 1-4 ring heteroatoms each independently O, N, or S, or —N(R^{E})—, wherein R^{E} is optionally substituted C₁₋₄ alkyl or optionally substituted 3-7 membered ring;
L² is absent, O, NH, —N(R^{E2})—, C(O), SO₂, an optionally substituted C₁₋₄ alkylene, an optionally substituted C₂₋₄ alkenylene, an optionally substituted C₁₋₄ alkynylene, optionally substituted C₁₋₄ heteroalkylene, or an optionally substituted 3-7 membered ring, wherein R^{E2} is optionally substituted C₁₋₄ alkyl or optionally substituted 3-7 membered ring;
R³⁰ is hydrogen, an optionally substituted phenyl, optionally substituted 5 or 6 membered heteroaryl, or optionally substituted 8-12 membered ring having two or more rings;
R⁹ at each occurrence is independently R^{N}, COOR^{N}, COR^{N}, CONH(R^{N}), or CON(R^{N})₂, wherein R^{N} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, or optionally substituted 5 or 6 membered heteroaryl; and
n1 is 0, 1, 2, or 3, preferably, n1 is 0 or 1, and R⁹ at each occurrence is independently CH₃, CH₂CH₃, CH₂CH₂CH₃, fluorine substituted C₁₋₃ alkyl (e.g., CF₂H), cyclopropyl, cyclobutyl, CH₂OH, CH₂OCH₃, CH₂OCH₂CH₃, CH₂NH₂, CH₂N₃, or CH₂NHC(O)OCH₃.

108. The compound of claim 107, or a pharmaceutically acceptable salt thereof, wherein Y is N.

109. The compound of claim 107, or a pharmaceutically acceptable salt thereof, wherein Y is CH.

110. The compound of any of claims 107-109, or a pharmaceutically acceptable salt thereof, wherein U is N.

111. The compound of any of claims 107-109, or a pharmaceutically acceptable salt thereof, wherein U is CR⁶.

112. The compound of claim 111, or a pharmaceutically acceptable salt thereof, wherein R⁶ is hydrogen.

113. The compound of claim 111, or a pharmaceutically acceptable salt thereof, wherein R⁶ is C₁₋₄ alkoxy or

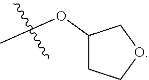 [SMILES: *OC1CCOC1]

114. The compound of any of claims 107-113, or a pharmaceutically acceptable salt thereof, wherein as applicable, R¹ is CN.

115. The compound of any of claims 107-113, or a pharmaceutically acceptable salt thereof, wherein as applicable, (i) R¹ is halogen, e.g., F or Cl; (ii) R¹ is 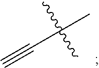 [SMILES: *C#C]
(iii) R¹ is an optionally substituted 5 or 6 membered heteroaryl having 1-4 ring heteroatoms independently selected from O, S, and N, such as an optionally substituted pyrimidinyl or an optionally substituted thiazolyl, for example, R¹ is 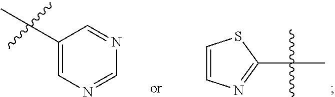 [SMILES: *c1cncnc1 ; *c1nccs1]
or (iv) R¹ is SR^{A1}, wherein R^{A1} is independently optionally substituted C₁₋₄ alkyl, for example, R¹ is SCH₃.

116. The compound of claim 107, or a pharmaceutically acceptable salt thereof, characterized as having a structure according to Formula III-4-C-1, III-4-C-2, III-4-C-3, III-4-C-4, III-4-D-1, or III-4-D-2

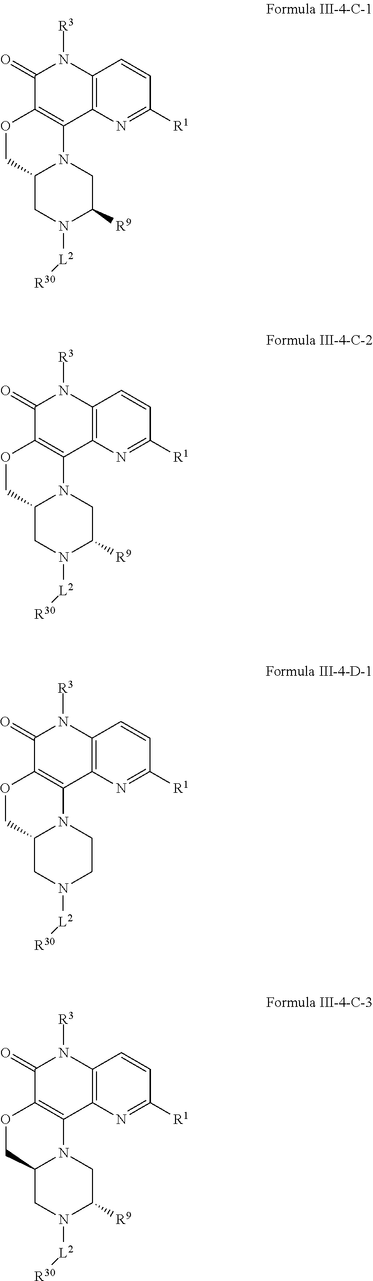

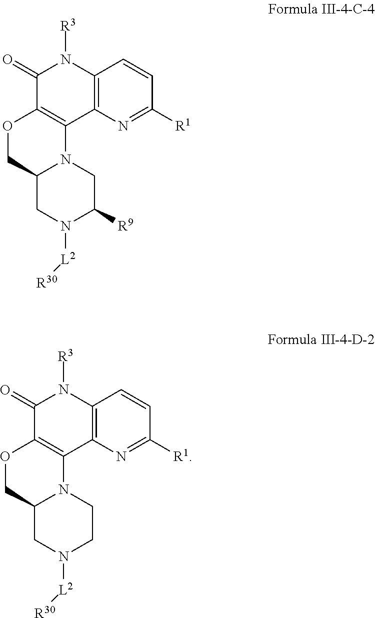

117. The compound of any of claims 107-115, or a pharmaceutically acceptable salt thereof, wherein R² is hydrogen.

118. The compound of any of claims 107-117, or a pharmaceutically acceptable salt thereof, wherein R³ is an optionally substituted C₁₋₄ alkyl, such as CH₃, CD₃ etc.

119. The compound of any of claims 107-118, or a pharmaceutically acceptable salt thereof, wherein Z is C(O).

120. The compound of any of claims 107-119, or a pharmaceutically acceptable salt thereof, wherein as applicable, R⁹ at each occurrence is independently an optionally substituted C₁₋₄ alkyl or optionally substituted C₃₋₆ cycloalkyl, preferably, CH₃, CH₂CH₃, CH₂CH₂CH₃, fluorine-substituted C₁₋₃ alkyl (e.g., CF₂H), cyclopropyl, cyclobutyl, CH₂OH, CH₂OCH₃, CH₂OCH₂CH₃, CH₂NH₂, CH₂N₃, or CH₂NHC(O)OCH₃.

121. The compound of any of claims 107-120, or a pharmaceutically acceptable salt thereof, wherein as applicable, X is N.

122. The compound of any of claims 107-120, or a pharmaceutically acceptable salt thereof, wherein as applicable, X is CR⁵, wherein R⁵ is CN.

123. The compound of any of claims 107-120, or a pharmaceutically acceptable salt thereof, wherein as applicable, X is CR⁵, wherein R⁵ is CONH₂, COOH, CONH(R^{C1}), CON(R^{C1})₂, or COOR^{C1}, wherein R¹ at each occurrence is independently a C₁₋₄ alkyl, such as methyl.

124. The compound of any of claims 107-123, or a pharmaceutically acceptable salt thereof, wherein as applicable, L¹ is 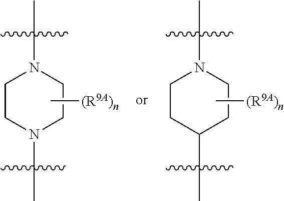
(either of the two attaching points of the piperidine, N or C, can be attached to L²),
wherein:
n is 0, 1, 2, 3, or 4, and
(1) R^{9A} at each occurrence is independently R^{N}, COOR^{N}, COR^{N}, CONH(R^{N}), or CON(R^{N})₂, wherein R^{N} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, or optionally substituted 5 or 6 membered heteroaryl; or
(2) two instances of R^{A} can be joined to form a double bond or a 3-5 membered ring and any remaining instance(s) of R^{A} are defined as in (1).

125. The compound of claim 124, or a pharmaceutically acceptable salt thereof, wherein R^{9A} at each occurrence is independently CH₃, CH₂CH₃, CH₂CH₂CH₃, fluorine-substituted C₁₋₃ alkyl (e.g., CF₂H), cyclopropyl, cyclobutyl, CH₂OH, CH₂OCH₃, CH₂OCH₂CH₃, CH₂NH₂, CH₂N₃, or CH₂NHC(O)OCH₃.

126. The compound of any of claims 107-123, or a pharmaceutically acceptable salt thereof, wherein as applicable, L¹ is selected from the following (the bottom attaching point, N or C, is attached to L²):

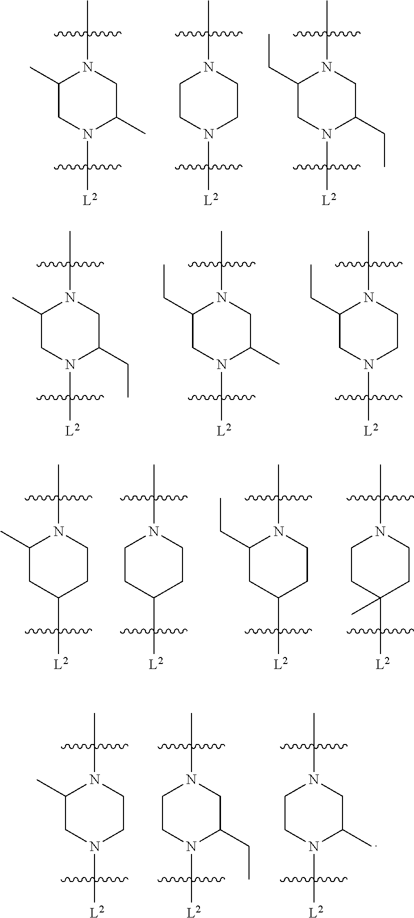

127. The compound of any of claims 107-123, or a pharmaceutically acceptable salt thereof, wherein as applicable, L¹ is selected from the following (the bottom attaching point, N or C, is attached to L²):

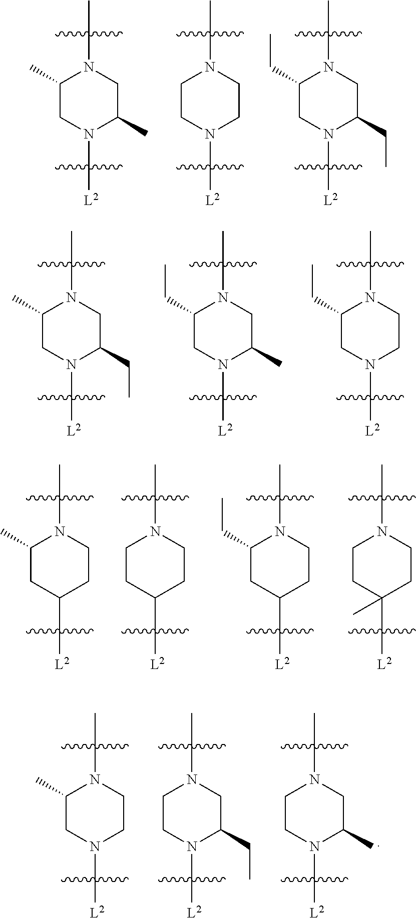

128. The compound of any of claims 107-123, or a pharmaceutically acceptable salt thereof, wherein as applicable, L¹ is an optionally substituted 7-12 membered heterocyclylene having two or more rings and 1-4 ring heteroatoms each independently O, N, or S, when substituted, the substituent(s) can be attached to any one or more of the two or more rings.

129. The compound of any of claims 107-123, or a pharmaceutically acceptable salt thereof, wherein as applicable, L¹ is selected from the following bicyclic heterocyclylene (the bottom attaching point, N or C, is attached to L²): 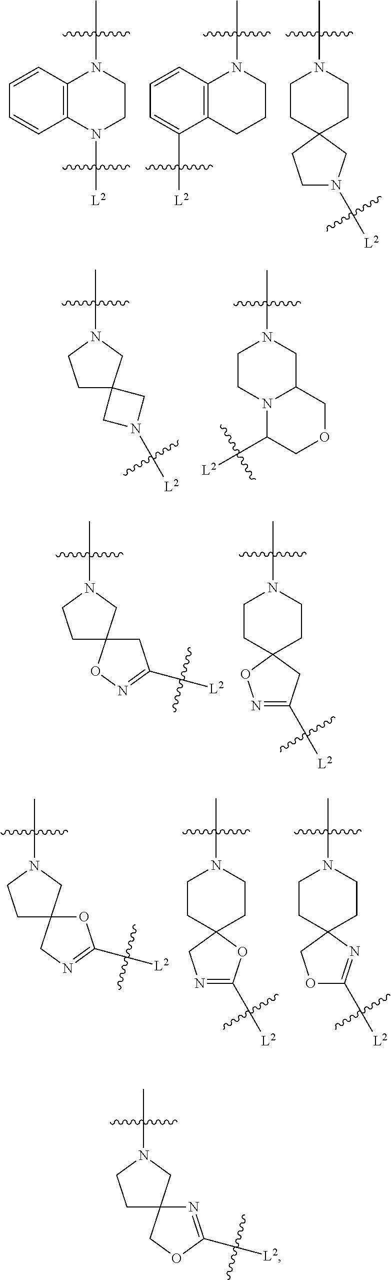
wherein in each of the bicyclic heterocyclylene, each of the two rings can be optionally substituted with 1-3 R¹⁰, wherein R¹⁰ at each occurrence is independently halogen, OH, NH₂, oxo (as applicable), R, OR, CN, NH(R), or N(R)₂, wherein R^{J} at each occurrence is independently an optionally substituted C₁₋₄ alkyl, optionally substituted C₂₋₄ alkenyl, optionally substituted C₂₋₄ alkynyl, or optionally substituted 3-4 membered ring.

130. The compound of any of claims 107-123, or a pharmaceutically acceptable salt thereof, wherein as applicable, L¹ is selected from the following bicyclic heterocyclylene (the bottom attaching point, N or C, is attached to L²): 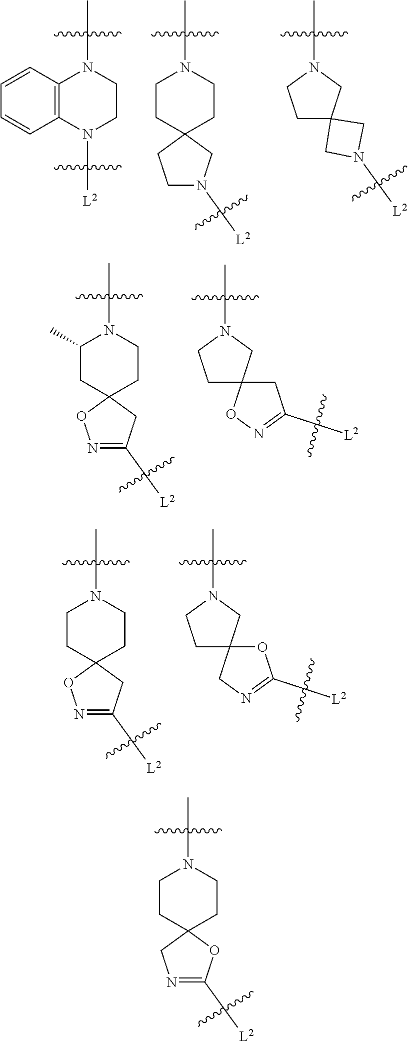
or L¹ is selected from

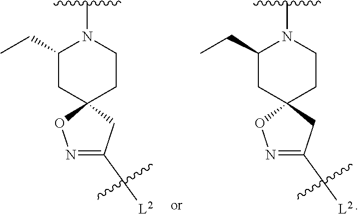

131. The compound of any of claims 107-123, or a pharmaceutically acceptable salt thereof, wherein as applicable, L¹ is —N(C₁₋₄ alkyl)-, such as —N(CH₃)—.

132. The compound of any of claims 107-131, or a pharmaceutically acceptable salt thereof, wherein L² is absent.

133. The compound of any of claims 107-131, or a pharmaceutically acceptable salt thereof, wherein L² is an optionally substituted C₁₋₄ alkylene selected from the following: 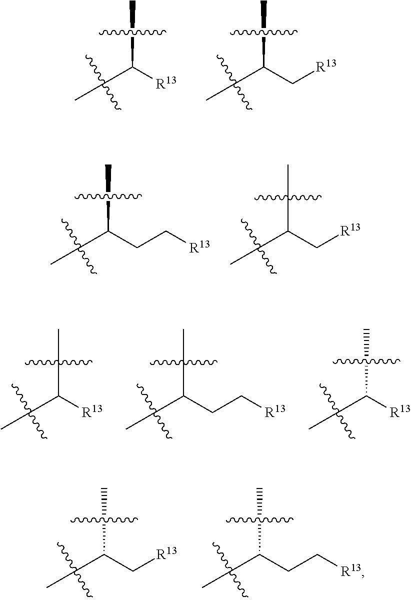
wherein:
R¹³ is selected from hydrogen, halogen (e.g., F, Cl, or Br), CN, OH, NH₂, COOH, CONH₂, SO₂NH₂, COR^{K}, COOR^{K}, CONH(R^{K}), CON(R^{K})₂, SO₂R^{K}, SO₂NH(R^{K}), SO₂N(R^{K})₂, R^{K}, OR^{K}, NH(R^{K}), N(R^{K})₂, SR^{K}, SOR^{K}, SO₂R^{K}, or P(O)(R^{K})₂, wherein R^{K} at each occurrence is independently optionally substituted C₁₋₄ alkyl, optionally substituted C₂₋₄ alkenyl, optionally substituted C₂₋₄ alkynyl, optionally substituted C₁₋₄ heteroalkyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl, wherein, when substituted, the optionally substituted C₁₋₄ alkyl, C₂₋₄ alkenyl, C₂₋₄ alkynyl, C₁₋₄ heteroalkyl, C₃₋₆ cycloalkyl, phenyl, 5 or 6 membered heteroaryl, or 4-7 membered heterocyclyl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

134. The compound of claim 133, or a pharmaceutically acceptable salt thereof, wherein R¹³ is hydrogen.

135. The compound of claim 133, or a pharmaceutically acceptable salt thereof, wherein R¹³ is CN, OH, COOH, CONH₂, methoxy, ethoxy, cyclopropyl, cyclobutyl, optionally substituted phenyl, or optionally substituted 5 or 6 membered heteroaryl having 1-3 ring heteroatoms independently selected from N, O, and S, wherein, when substituted, the optionally substituted phenyl or 5 or 6 membered heteroaryl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

136. The compound of any of claims 107-131, or a pharmaceutically acceptable salt thereof, wherein L² is O or C(O).

137. The compound of any of claims 107-131, or a pharmaceutically acceptable salt thereof, wherein L² is an optionally substituted 5 or 6 membered heteroarylene, for example,

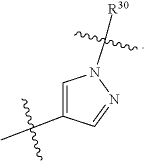

138. The compound of any of claims 107-123, or a pharmaceutically acceptable salt thereof, wherein as applicable, L¹ is —N(R^{E})—, L² is an optionally substituted phenylene, e.g. 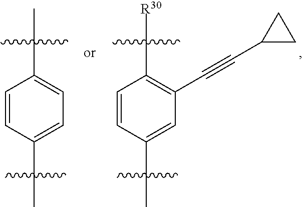
and R³⁰ is hydrogen.

139. The compound of any of claims 107-123, or a pharmaceutically acceptable salt thereof, wherein as applicable, L¹, L², and R³⁰ together is:

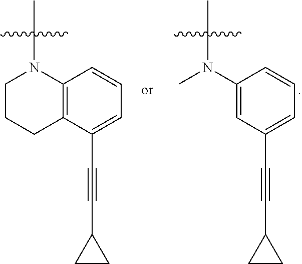 [SMILES: *N1CCCc2c(C#CC3CC3)cccc21 ; *N(C)c1cccc(C#CC2CC2)c1]

140. The compound of any of claims 107-137, or a pharmaceutically acceptable salt thereof, wherein R³⁰ is optionally substituted phenyl, such as a phenyl which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl.

141. The compound of claim 140, or a pharmaceutically acceptable salt thereof, wherein R³⁰ is a phenyl which is substituted with 1-3 (e.g., 1 or 2) R²², wherein R²² at each occurrence is independently halogen (e.g., F, Cl, or Br), CN, OH, R^{M1}, OR^{M1}, SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

142. The compound of claim 140, or a pharmaceutically acceptable salt thereof, wherein R³⁰ is a phenyl which is substituted with 1-3 (e.g., 1 or 2) R²², wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

143. The compound of any of claims 107-137, or a pharmaceutically acceptable salt thereof, wherein R³⁰ is optionally substituted 5 or 6-membered heteroaryl, such as a pyridyl 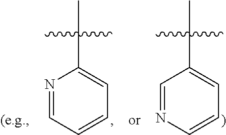 [SMILES: *c1ccccn1 ; *c1cccnc1]
which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl.

144. The compound of claim 143, or a pharmaceutically acceptable salt thereof, wherein R³⁰ is a 5-membered heteroaryl 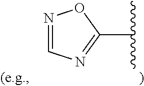 [SMILES: *c1ncno1]
or pyridyl 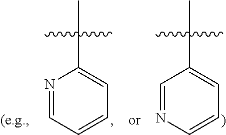 [SMILES: *c1ccccn1 ; *c1cccnc1]
which is substituted with 1-3 (e.g., 1 or 2) R²², as valency permits, wherein R²² at each occurrence is independently halogen (e.g., F, Cl, or Br), CN, OH, R^{M1}, OR^{M1}, SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

145. The compound of claim 143, or a pharmaceutically acceptable salt thereof, wherein R³⁰ is a 5-membered heteroaryl 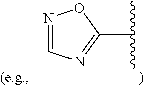 [SMILES: *c1ncno1]
or pyridyl 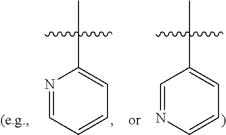 [SMILES: *c1ccccn1 ; *c1cccnc1]
which is substituted with 1-3 (e.g., 1 or 2) R²², as valency permits, wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

146. The compound of any of claims 107-137, or a pharmaceutically acceptable salt thereof, wherein R³⁰ has a structure according to S-1-A, S-1-B, or S-1-C: 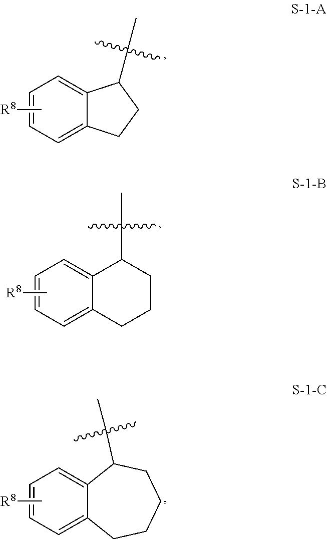
wherein R⁸ is C₁₋₄ alkyl optionally substituted with 1-3 F (such as CH₃, CF₃, etc.), C₁₋₄ alkoxy optionally substituted with 1-3 F (such as OCH₃, OCF₃, etc.), SCF₃, SF₅, cyclopropyl, cyclobutyl, or

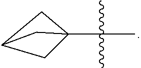 [SMILES: *C12CC(C1)C2]

147. The compound of any of claims 107-123, or a pharmaceutically acceptable salt thereof, wherein L¹, L², and R³⁰ together (i.e., L¹-L²-R³⁰) is selected from: 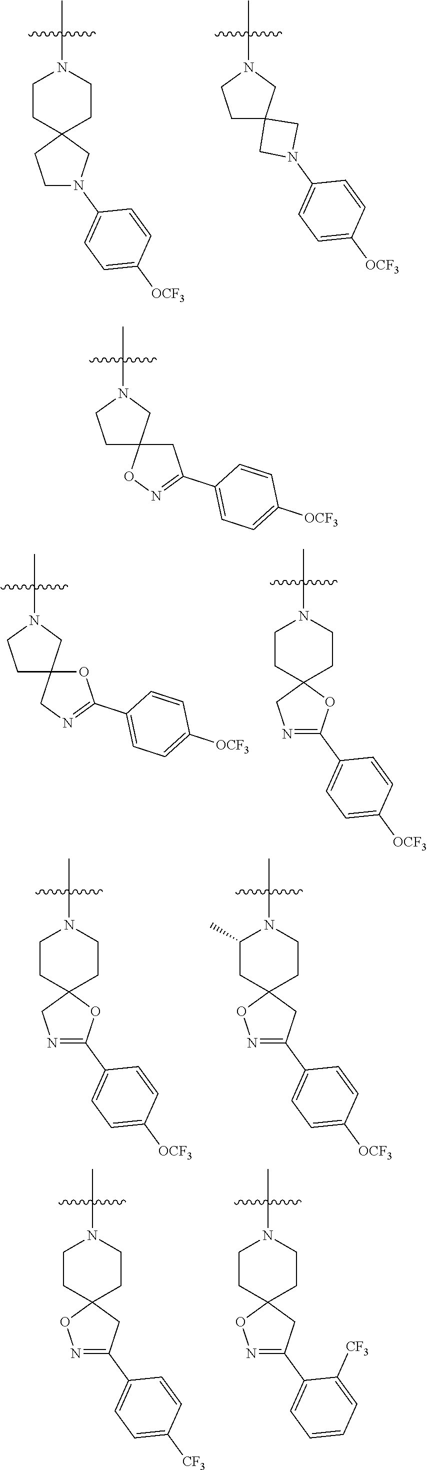 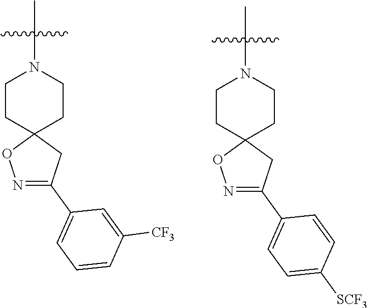
or L¹-L²-R³⁰ is selected from

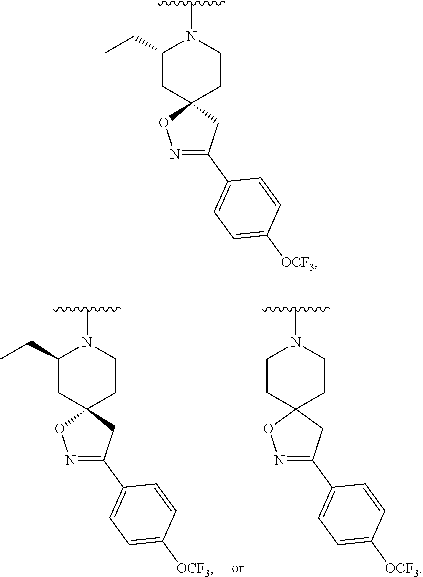 [SMILES: *N1CC[C@]2(CC(c3ccc(OC(F)(F)F)cc3)=NO2)C[C@@H]1CC ; *N1CC[C@@]2(CC(c3ccc(OC(F)(F)F)cc3)=NO2)C[C@H]1CC ; *N1CCC2(CC1)CC(c1ccc(OC(F)(F)F)cc1)=NO2]

148. The compound of any of claims 107-123, or a pharmaceutically acceptable salt thereof, wherein L¹, L², and R³⁰ together is selected from: 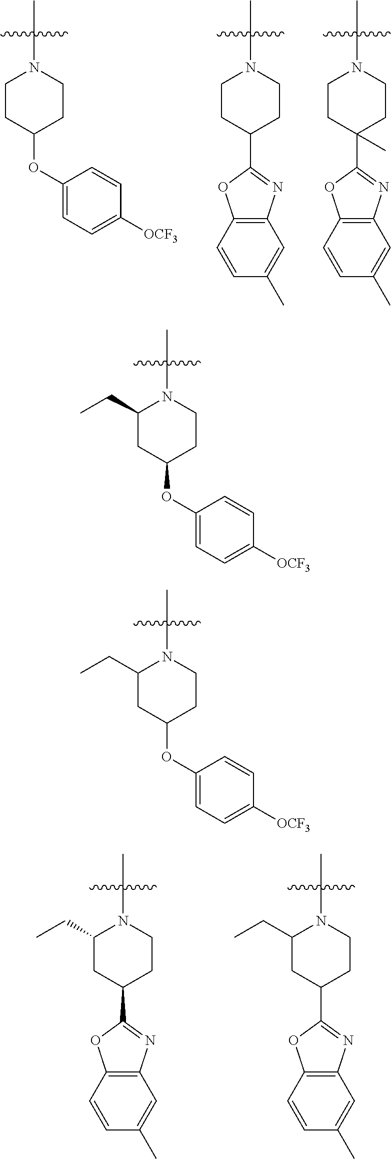
or L¹-L²-R³⁰ is selected from

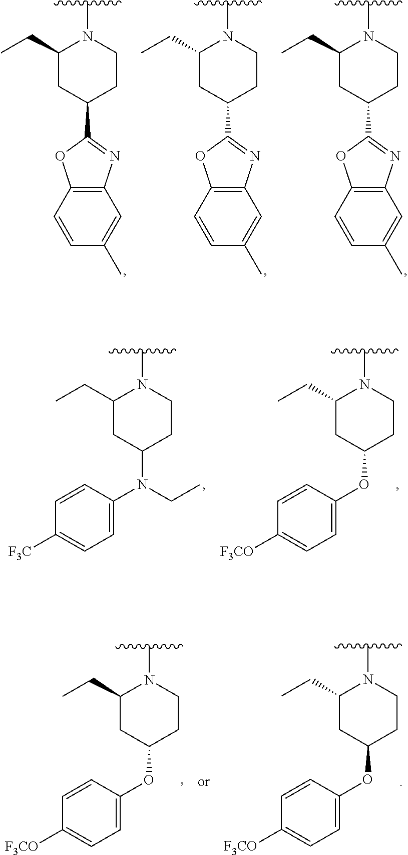

149. The compound of any of claims 107-123, or a pharmaceutically acceptable salt thereof, wherein L¹, L², and R³⁰ together is selected from: 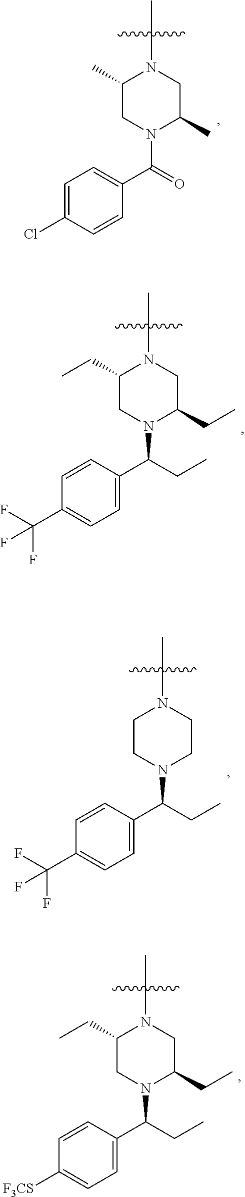 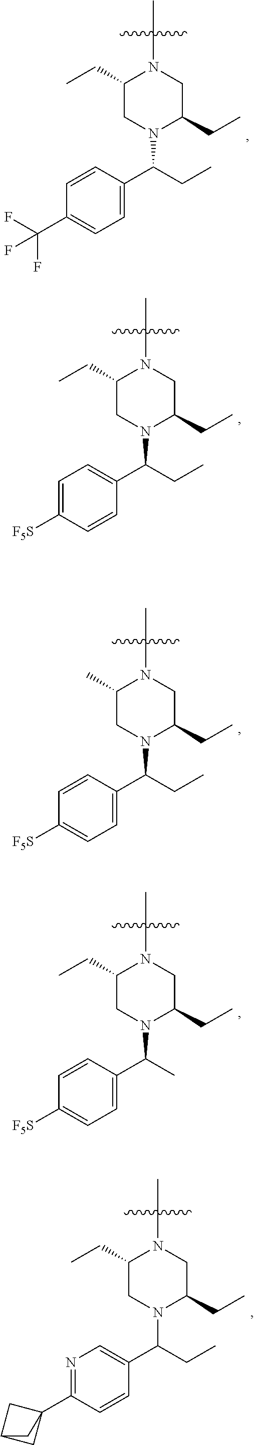 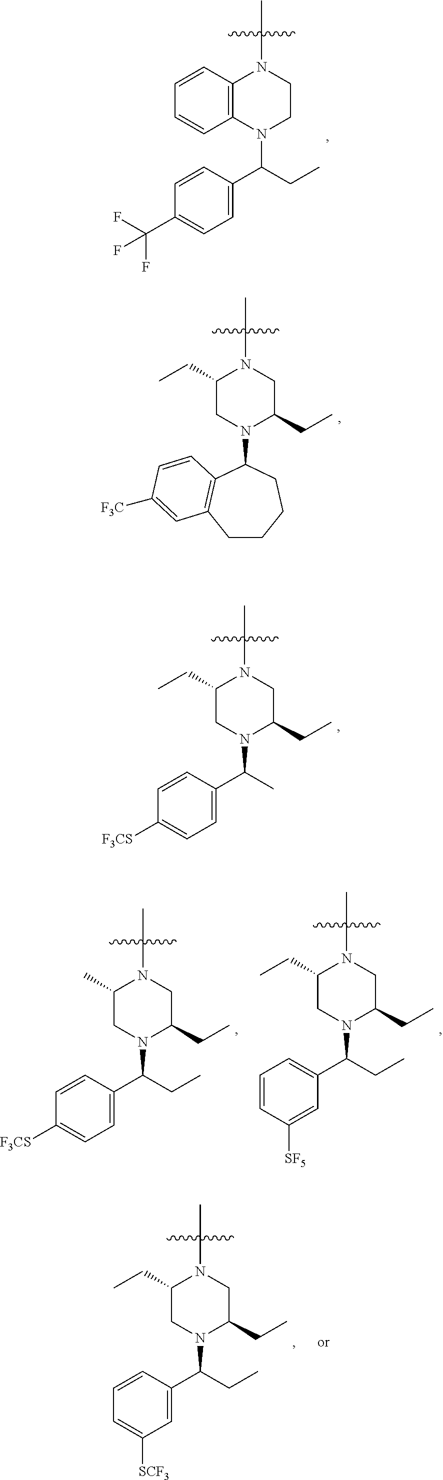 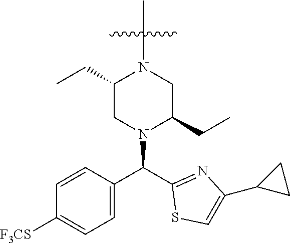
or L¹-L²-R³⁰ is selected from 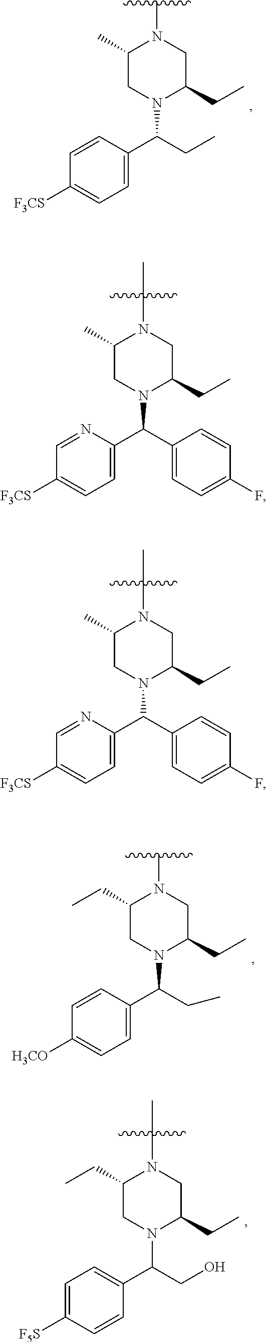 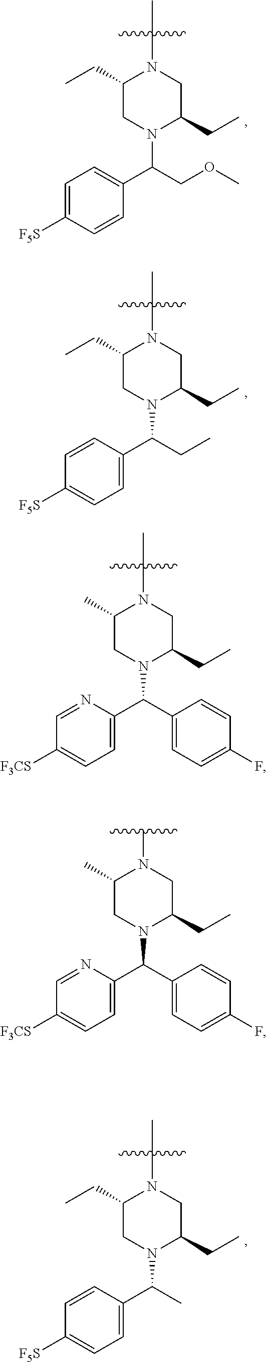 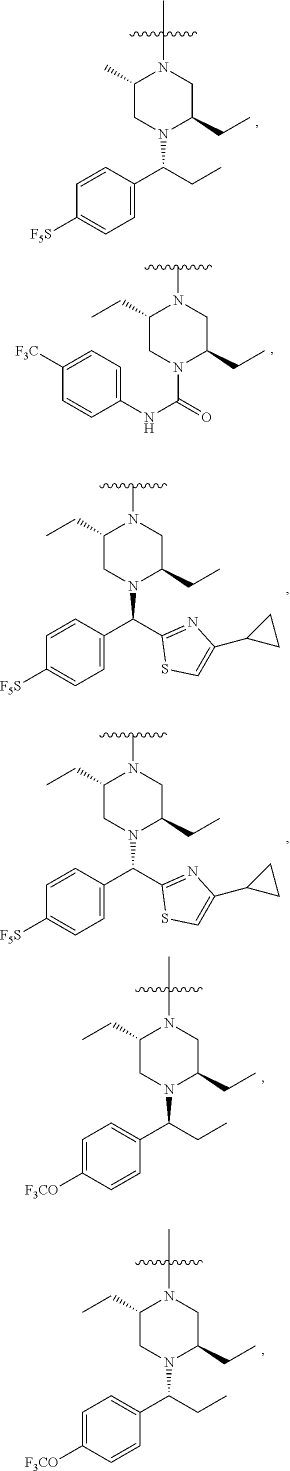 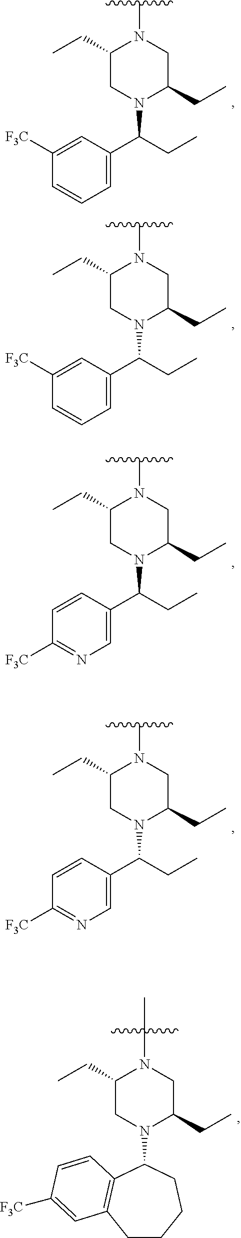 [SMILES: *N1C[C@@H](CC)N([C@@H](CC)c2cccc(C(F)(F)F)c2)C[C@@H]1CC ; *N1C[C@@H](CC)N([C@H](CC)c2cccc(C(F)(F)F)c2)C[C@@H]1CC ; *N1C[C@@H](CC)N([C@@H](CC)c2ccc(C(F)(F)F)nc2)C[C@@H]1CC ; *N1C[C@@H](CC)N([C@H](CC)c2ccc(C(F)(F)F)nc2)C[C@@H]1CC ; *N1C[C@@H](CC)N([C@@H]2CCCCc3cc(C(F)(F)F)ccc32)C[C@@H]1CC] 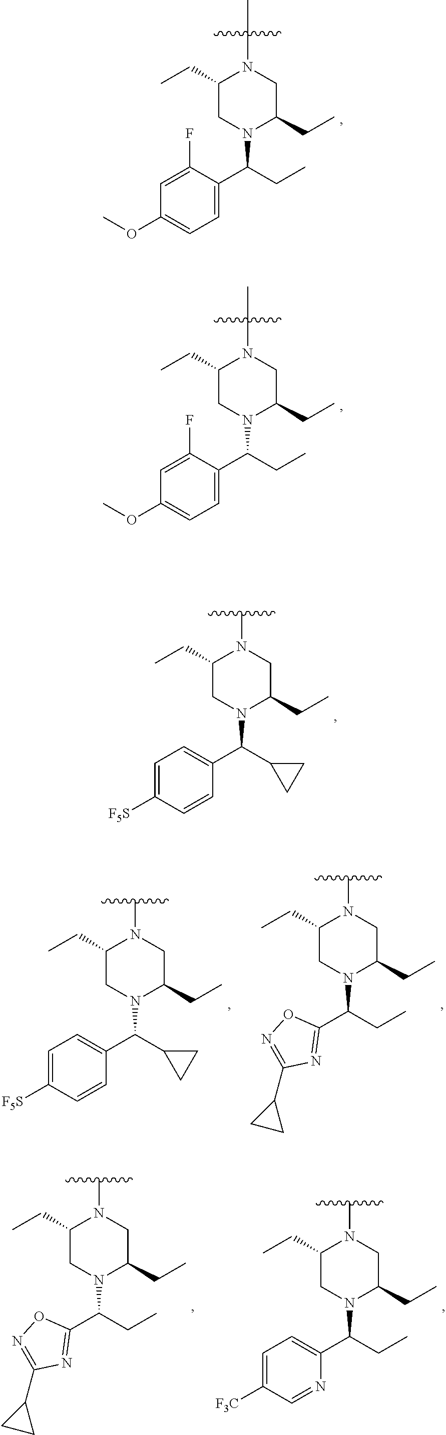 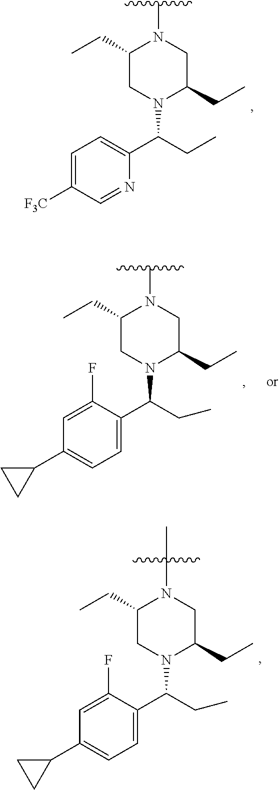 [SMILES: *N1C[C@@H](CC)N([C@H](CC)c2ccc(C(F)(F)F)cn2)C[C@@H]1CC ; *N1C[C@@H](CC)N([C@@H](CC)c2ccc(C3CC3)cc2F)C[C@@H]1CC ; *N1C[C@@H](CC)N([C@H](CC)c2ccc(C3CC3)cc2F)C[C@@H]1CC]
or L¹, L², and R³⁰ together is selected from:

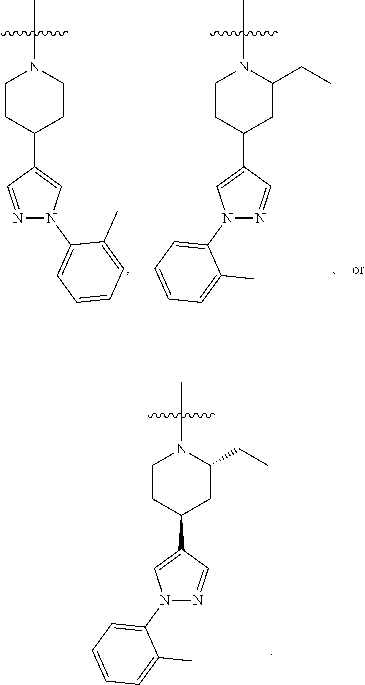

150. The compound of any of claims 107-137, or a pharmaceutically acceptable salt thereof, wherein R³⁰ is selected from the following: 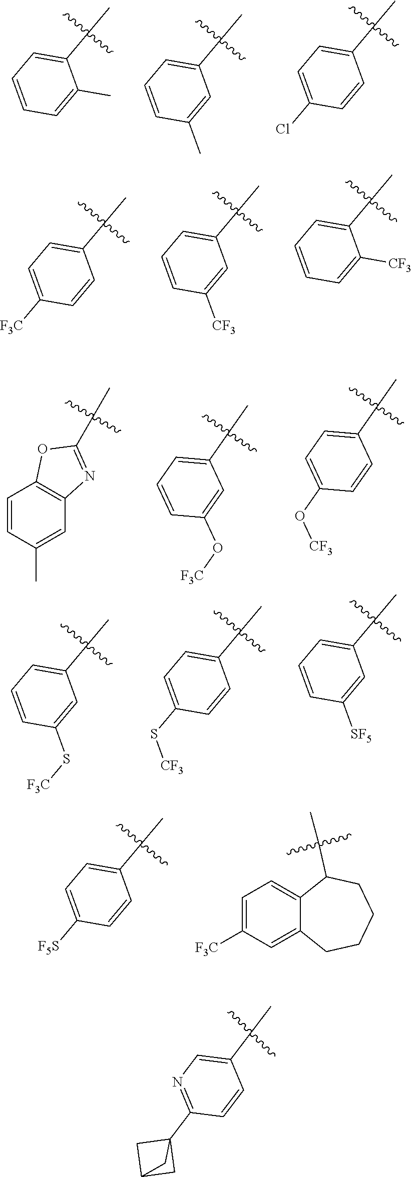
or R³⁰ is selected from:

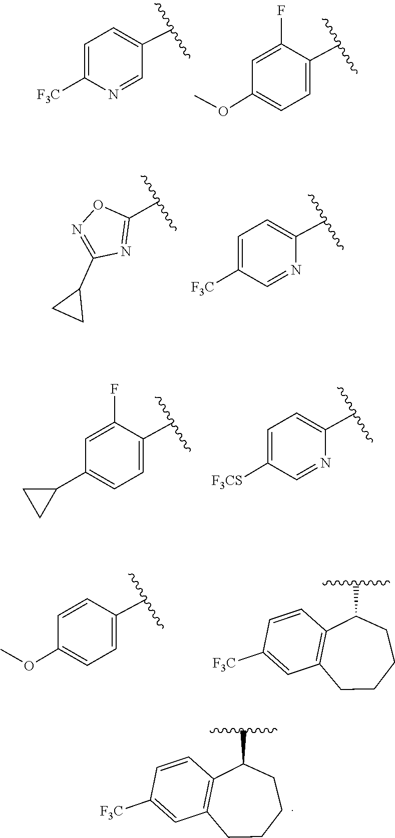

151. A compound of Formula IV, or a pharmaceutically acceptable salt thereof, 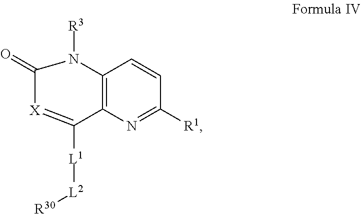
wherein:
R¹ is hydrogen, halogen (e.g., F, Cl, or Br), CN, OH, NH₂, COOH, CONH₂, R^{A}, OR^{A}, NH(R^{A}), N(R^{A})₂, COOR^{A}, CONH(R^{A}), CON(R^{A})₂, SR^{A}, SOR^{A}, SO₂R^{A}, or P(O)(R^{A})₂, wherein R^{A} at each occurrence is independently optionally substituted C₁4 alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
R³ is hydrogen, optionally substituted C₁₋₄ alkyl, optionally substituted C₃₋₆ cycloalkyl, or optionally substituted 4-7 membered heterocyclyl (such as a saturated 4-7 membered heterocyclyl);
X is N or CR⁵, wherein R⁵ is hydrogen, halogen, CN, CONH₂, COOH, CONH(R^{C}), CON(R^{C})₂, OR^{C}, NO₂, or COOR^{C}, wherein R^{C} at each occurrence is independently an optionally substituted C₁₋₄ alkyl;
L¹ and L² are characterized in that:
i) L¹ is a 6-12 membered heterocyclylene having two or more rings and 1-4 ring heteroatoms each independently O, N, or S, wherein the heterocyclylene is optionally substituted; and L² is absent, O, C(O), an optionally substituted C₁₋₄ alkylene, an optionally substituted C₂₋₄ alkenylene, an optionally substituted C₁₋₄ alkynylene, optionally substituted C₁₋₄ heteroalkylene, or an optionally substituted 3-7 membered ring;
ii) L¹ is a 4-7 membered monocyclic heterocyclylene having 1-2 ring heteroatoms each independently O, N, or S, wherein the monocyclic heterocyclylene is optionally substituted; and L² is absent, O, C(O), or an optionally substituted 3-7 membered ring or L² is N(C₁₋₄ alkyl); or
iii) L¹ is —N(R^{E})—, wherein R^{E} is optionally substituted C₁₋₄ alkyl or optionally substituted 3-7 membered ring; and L² is absent or an optionally substituted 3-7 membered ring; and
R³⁰ is hydrogen, an optionally substituted phenyl, optionally substituted 5 or 6 membered heteroaryl, or optionally substituted 8-12 membered ring having two or more rings.

152. The compound of claim 151, or a pharmaceutically acceptable salt thereof, wherein R¹ is CN.

153. The compound of claim 151, or a pharmaceutically acceptable salt thereof, wherein (i) R¹ is halogen, e.g., F or Cl; (ii) R¹ is 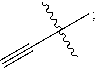 [SMILES: *C#C]
(iii) R¹ is an optionally substituted 5 or 6 membered heteroaryl having 1-4 ring heteroatoms independently selected from O, S, and N, such as an optionally substituted pyrimidinyl or an optionally substituted thiazolyl, for example, R¹ is 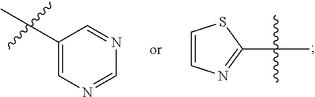 [SMILES: *c1cncnc1 ; *c1nccs1]
or (iv) R¹ is SR^{A1}, wherein R^{A1} is independently optionally substituted C₁₋₄ alkyl, for example, R¹ is SCH₃.

154. The compound of any of claims 151-153, or a pharmaceutically acceptable salt thereof, wherein R³ is an optionally substituted C₁₋₄ alkyl, such as CH₃, CD₃ etc.

155. The compound of any of claims 151-154, or a pharmaceutically acceptable salt thereof, wherein X is N.

156. The compound of any of claims 151-154, or a pharmaceutically acceptable salt thereof, wherein X is CR⁵, wherein R⁵ is CN.

157. The compound of any of claims 151-154, or a pharmaceutically acceptable salt thereof, wherein X is CR⁵, wherein R⁵ is CONH₂, COOH, CONH(R^{C1}), CON(R^{C1})₂, or COOR^{C1} wherein R¹ at each occurrence is independently a C₁₋₄ alkyl, such as methyl.

158. The compound of any of claims 151-157, or a pharmaceutically acceptable salt thereof, wherein R³⁰ is optionally substituted phenyl, such as a phenyl which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from 0, S, and N, or optionally substituted 4-7 membered heterocyclyl.

159. The compound of claim 158, or a pharmaceutically acceptable salt thereof, wherein R³⁰ is a phenyl which is substituted with 1-3 (e.g., 1 or 2) R²², wherein R²² at each occurrence is independently halogen (e.g., F, Cl, or Br), CN, OH, R^{M1}, OR^{M1}, SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

160. The compound of claim 158, or a pharmaceutically acceptable salt thereof, wherein R³⁰ is a phenyl which is substituted with 1-3 (e.g., 1 or 2) R²², wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

161. The compound of any of claims 151-157, or a pharmaceutically acceptable salt thereof, wherein R³⁰ is optionally substituted 5 or 6-membered heteroaryl, such as a pyridyl 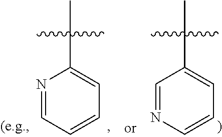 [SMILES: *c1ccccn1 ; *c1cccnc1]
which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl.

162. The compound of claim 160, or a pharmaceutically acceptable salt thereof, wherein R³⁰ is a 5-membered heteroaryl 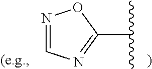 [SMILES: *c1ncno1]
or pyridyl 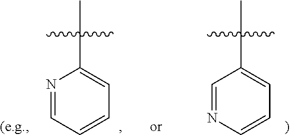 [SMILES: *c1ccccn1 ; *c1cccnc1]
which is substituted with 1-3 (e.g., 1 or 2) R²², as valency permits, wherein R²² at each occurrence is independently halogen (e.g., F, Cl, or Br), CN, OH, R^{M1}, OR^{M1}, SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

163. The compound of claim 160, or a pharmaceutically acceptable salt thereof, wherein R³⁰ is a 5-membered heteroaryl 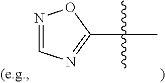 [SMILES: *c1ncno1]
or pyridyl 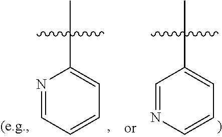 [SMILES: *c1ccccn1 ; *c1cccnc1]
which is substituted with 1-3 (e.g., 1 or 2) R²², as valency permits, wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

164. The compound of any of claims 151-157, or a pharmaceutically acceptable salt thereof, wherein R³⁰ has a structure according to S-1-A, S-1-B, or S-1-C: 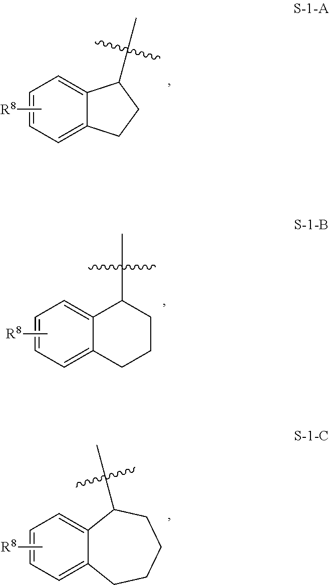
wherein R⁸ is C₁₋₄ alkyl optionally substituted with 1-3 F (such as C₃, C3, etc.) C₁₋₄ alkoxy optionally substituted with 1-3 F (such as OCH₃, OCF₃, etc.), SCF₃, SF₅, cyclopropyl, cyclobutyl, or

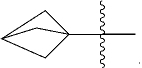 [SMILES: *C12CC(C1)C2]

165. The compound of any of claims 151-157, or a pharmaceutically acceptable salt thereof, wherein L¹, L², and R³⁰ together is selected from: 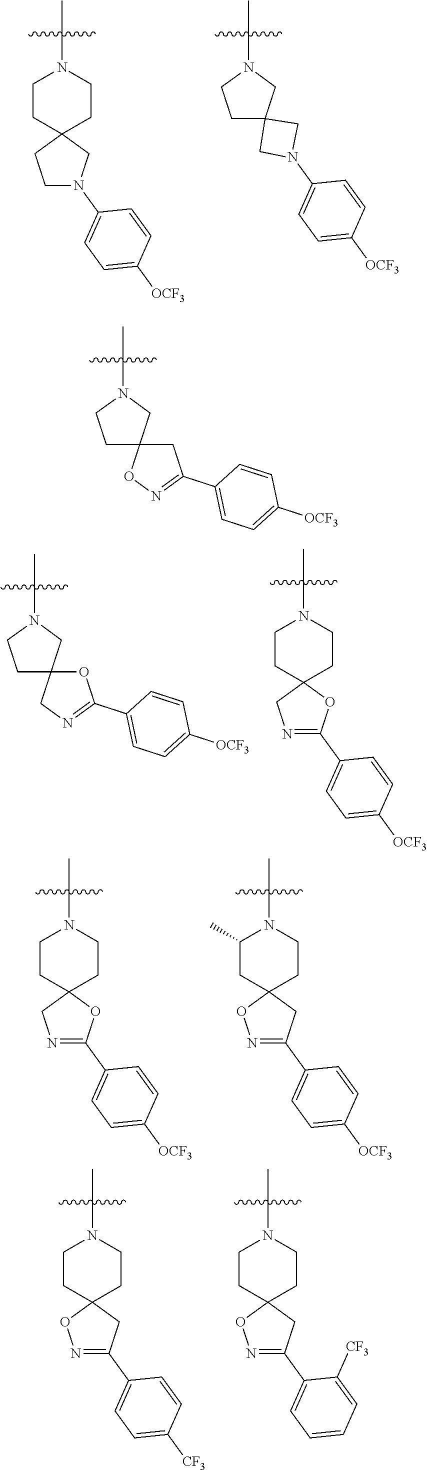 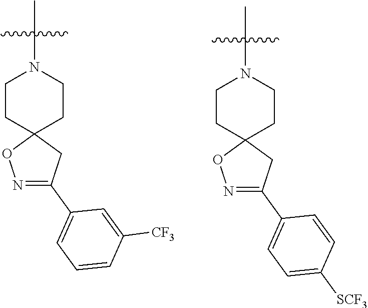
or L¹-L²-R³⁰ is selected from

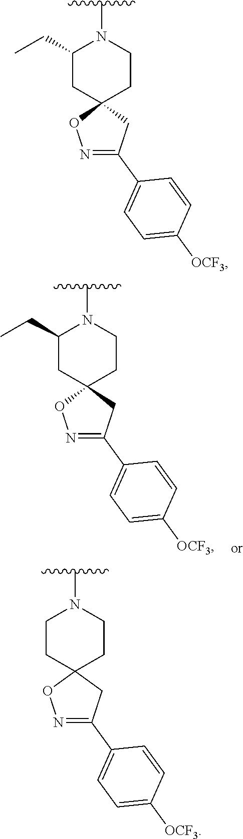 [SMILES: *N1CC[C@]2(CC(c3ccc(OC(F)(F)F)cc3)=NO2)C[C@@H]1CC ; *N1CC[C@@]2(CC(c3ccc(OC(F)(F)F)cc3)=NO2)C[C@H]1CC ; *N1CCC2(CC1)CC(c1ccc(OC(F)(F)F)cc1)=NO2]

166. The compound of any of claims 151-157, or a pharmaceutically acceptable salt thereof, wherein L¹, L², and R³⁰ together is selected from: 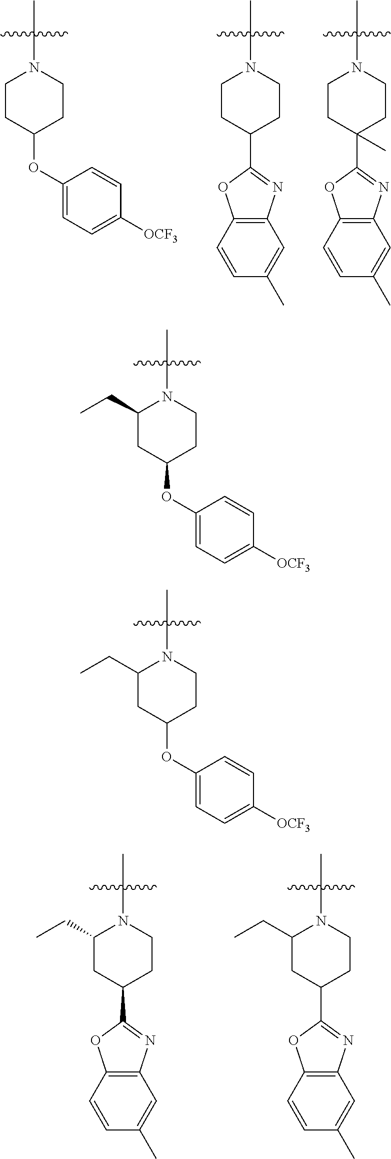
or L¹-L²-R³⁰ is selected from

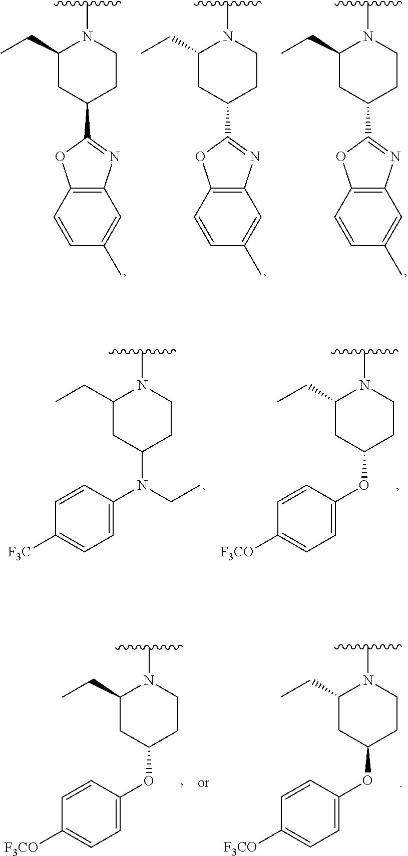

167. The compound of any of claims 151-157, or a pharmaceutically acceptable salt thereof, wherein L¹, L², and R³⁰ together is selected from:

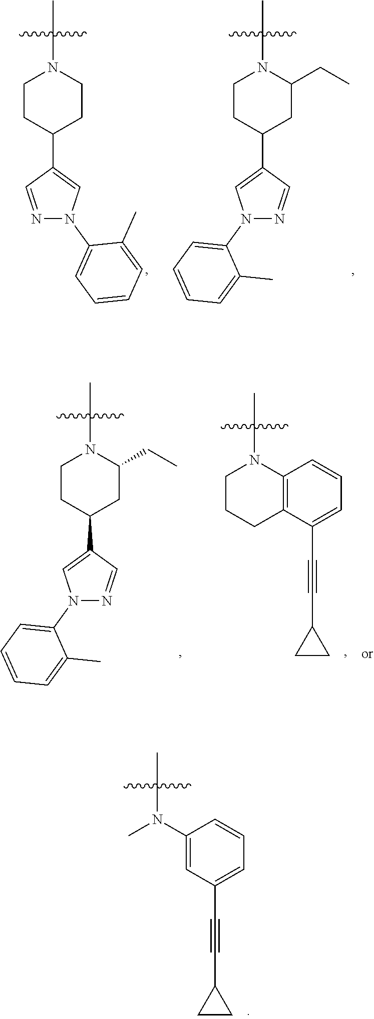

168. A compound of Formula V, or a pharmaceutically acceptable salt thereof, 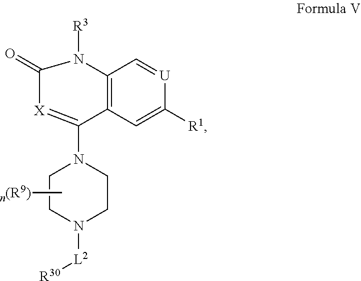
wherein:
R¹ is hydrogen, halogen (e.g., F, Cl, or Br), CN, OH, NH₂, COOH, CONH₂, R^{A}, OR^{A}, NH(R^{A}), N(R^{A})₂, COOR^{A}, CONH(R^{A}), CON(R^{A})₂, SR^{A}, SOR^{A}, SO₂R^{A}, or P(O)(R^{A})₂, wherein R^{A} at each occurrence is independently optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
R³ is hydrogen, optionally substituted C₁₋₄ alkyl, optionally substituted C₃₋₆ cycloalkyl, or optionally substituted 4-7 membered heterocyclyl (such as a saturated 4-7 membered heterocyclyl);
X is N or CR⁵, wherein R⁵ is hydrogen, halogen, CN, CONH₂, COOH, CONH(R^{C}), CON(R^{C})₂, OR^{C}, NO₂, or COOR^{C}, wherein R^{C} at each occurrence is independently an optionally substituted C₁₋₄ alkyl;
U is N or CR⁶, wherein R⁶ is hydrogen, halogen (e.g., F, Cl, or Br), CN, OH, NH₂, R^{D}, OR^{D}, NH(R^{D}), N(R^{D})₂, COOH, CONH₂, COOR^{D}, CONH(R^{D}), CON(R^{D})₂, SR^{D}, SOR^{D}, SO₂R^{D}, or P(O)(R^{D})₂, wherein R^{D} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted 5 or 6 membered heteroaryl having 1-4 ring heteroatoms independently selected from O, S, and N, or optionally substituted 4-7 membered heterocyclyl;
R⁹ at each occurrence is independently R^{N}, COOR^{N}, COR^{N}, CONH(R^{N}), or CON(R^{N})₂, wherein R^{N} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, or optionally substituted 5 or 6 membered heteroaryl;
n is 0, 1, 2, 3, or 4;
L² is absent, C(O), SO₂, an optionally substituted C₁₋₄ alkylene, an optionally substituted C₂₋₄ alkenylene, an optionally substituted C₁₋₄ alkynylene, optionally substituted C₁₋₄ heteroalkylene, or an optionally substituted 3-7 membered ring; and
R³⁰ is hydrogen, an optionally substituted phenyl, optionally substituted 5 or 6 membered heteroaryl, or optionally substituted 8-12 membered ring having two or more rings.

169. The compound of claim 168, or a pharmaceutically acceptable salt thereof, wherein U is N.

170. The compound of claim 168, or a pharmaceutically acceptable salt thereof, wherein U is CR⁶.

171. The compound of claim 170, or a pharmaceutically acceptable salt thereof, wherein R⁶ is hydrogen.

172. The compound of claim 170, or a pharmaceutically acceptable salt thereof, wherein R⁶ is halogen or OR^{D1}, wherein R^{D1} is optionally substituted C₁₋₄ alkyl, optionally substituted C₃₋₆ cycloalkyl, or optionally substituted 4-7 membered heterocyclyl having 1 or 2 ring heteroatoms independently selected from O, N, and S; wherein, when substituted, the optionally substituted C₁₋₄ alkyl, C₃₋₆ cycloalkyl, or 4-7 membered heterocyclyl can be substituted with one or more (e.g., 1-3) substituents, for example, each substituent can be independently selected from halogen (e.g., F), OH, CN, C₁₋₄ alkyl or C₁₋₄ alkoxy.

173. The compound of claim 170, or a pharmaceutically acceptable salt thereof, wherein R⁶ is C₁₋₄ alkoxy or

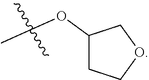 [SMILES: *OC1CCOC1]

174. The compound of any of claims 168-173, or a pharmaceutically acceptable salt thereof, wherein as applicable, R¹ is CN.

175. The compound of any of claims 168-173, or a pharmaceutically acceptable salt thereof, wherein as applicable, (i) R¹ is halogen, e.g., F or Cl; (ii) R¹ is 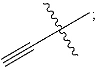 [SMILES: *C#C]
(iii) R¹ is an optionally substituted 5 or 6 membered heteroaryl having 1-4 ring heteroatoms independently selected from O, S, and N, such as an optionally substituted pyrimidinyl or an optionally substituted thiazolyl, for example, R¹ is 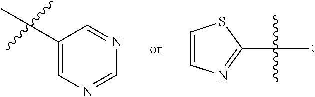 [SMILES: *c1cncnc1 ; *c1nccs1]
or (iv) R¹ is SR^{A1}, wherein R^{A1} is independently optionally substituted C₁₋₄ alkyl, for example, R¹ is SCH₃.

176. The compound of any of claims 168-175, or a pharmaceutically acceptable salt thereof, wherein R³ is an optionally substituted C₁₋₄ alkyl, such as CH₃, CD₃ etc.

177. The compound of any of claims 168-176, or a pharmaceutically acceptable salt thereof, wherein X is N.

178. The compound of any of claims 168-176, or a pharmaceutically acceptable salt thereof, wherein X is CR⁵, wherein R⁵ is CN.

179. The compound of any of claims 168-176, or a pharmaceutically acceptable salt thereof, wherein X is CR⁵, wherein R⁵ is CONH₂, COOH, CONH(R^{C1}), CON(R^{C1})₂, or COOR^{C1}wherein Rei at each occurrence is independently a C₁₋₄ alkyl, such as methyl.

180. The compound of any of claims 168-179, or a pharmaceutically acceptable salt thereof, wherein n is 0, 1, or 2.

181. The compound of any of claims 168-180, or a pharmaceutically acceptable salt thereof, wherein R⁹ at each occurrence is independently CH₃, CH₂CH₃, CH₂CH₂CH₃, fluorine-substituted C₁₋₃ alkyl (e.g., CF₂H), cyclopropyl, cyclobutyl, CH₂OH, CH₂OCH₃, CH₂OCH₂CH₃, CH₂NH₂, CH₂N₃, or CH₂NHC(O)OCH₃.

182. The compound of any of claims 168-181, or a pharmaceutically acceptable salt thereof, wherein L² is an optionally substituted C₁₋₄ alkylene selected from the following: 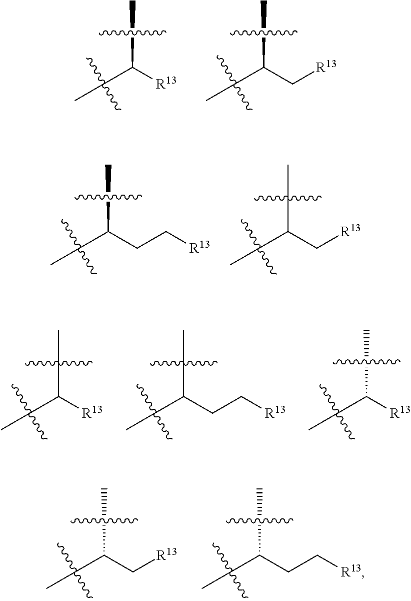
wherein:
R¹³ is selected from hydrogen, halogen (e.g., F, Cl, or Br), CN, OH, NH₂, COOH, CONH₂, SO₂NH₂, COR^{K}, COOR^{K}, CONH(R^{K}), CON(R^{K})₂, SO₂R^{K}, SO₂NH(R^{K}), SO₂N(R^{K})₂, R^{K}, OR^{K}, NH(R^{K}), N(R^{K})₂, SR^{K}, SOR^{K}, SO₂R^{K}, or P(O)(R^{K})₂, wherein R^{K} at each occurrence is independently optionally substituted C₁₋₄ alkyl, optionally substituted C₂₋₄ alkenyl, optionally substituted C₂₋₄ alkynyl, optionally substituted C₁₋₄ heteroalkyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl, wherein, when substituted, the optionally substituted C₁₋₄ alkyl, C₂₋₄ alkenyl, C₂₋₄ alkynyl, C₁₋₄ heteroalkyl, C₃₋₆ cycloalkyl, phenyl, 5 or 6 membered heteroaryl, or 4-7 membered heterocyclyl is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

183. The compound of any of claims 168-182, or a pharmaceutically acceptable salt thereof, wherein R³⁰ is optionally substituted phenyl, such as a phenyl which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl.

184. The compound of claim 183, or a pharmaceutically acceptable salt thereof, wherein R³⁰ is a phenyl which is substituted with 1-3 (e.g., 1 or 2) R²², wherein R²² at each occurrence is independently halogen (e.g., F, Cl, or Br), CN, OH, R^{M1}, OR^{M1}, SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

185. The compound of claim 183, or a pharmaceutically acceptable salt thereof, wherein R³⁰ is a phenyl which is substituted with 1-3 (e.g., 1 or 2) R²², wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

186. The compound of any of claims 168-182, or a pharmaceutically acceptable salt thereof, wherein R³⁰ is optionally substituted 5 or 6-membered heteroaryl, such as a pyridyl 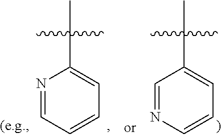 [SMILES: *c1ccccn1 ; *c1cccnc1]
which is substituted with 1-3 (e.g., 1 or 2) R²², wherein R²² at each occurrence is independently halogen (e.g., F, Cl, or Br), CN, OH, NH₂, R^{M}, OR^{M}, COOH, CONH₂, COOR^{M}, CONH(R^{M}), CON(R^{M})₂, NHCO(R^{M}), N(R^{M})CO(R^{M}), SO₂R^{M}, SO₂NH₂, S(O)(NH)R^{M}, S(O)(NR^{M})R^{M}, SO₂N(R^{M})₂, NHSO₂R^{M}, N(R^{M})SO₂R^{M}, P(O)(R^{M})₂, P(O)(OR^{M})₂, NH(R^{M}), N(R^{M})₂, SR^{M}, or SF₅, wherein R^{M} at each occurrence is independently an optionally substituted C₁₋₄ alkyl, optionally substituted C₂₋₄ alkenyl, optionally substituted C₂₋₄ alkynyl, optionally substituted C₃₋₆ cycloalkyl, optionally substituted phenyl, optionally substituted 5 or 6 membered heteroaryl having 1-4 ring heteroatoms independently selected from O, S, and N, or optionally substituted 4-7 membered heterocyclyl.

187. The compound of claim 186, or a pharmaceutically acceptable salt thereof, wherein R³⁰ is a 5-membered heteroaryl 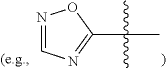 [SMILES: *c1ncno1]
or pyridyl 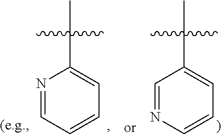 [SMILES: *c1ccccn1 ; *c1cccnc1]
which is substituted with 1-3 (e.g., 1 or 2) R²², as valency permits, wherein R²² at each occurrence is independently halogen (e.g., F, Cl, or Br), CN, OH, R^{M1}, OR^{M1}, SO₂R^{M1}, P(O)(R^{M1})₂, SR^{M1}, or SF₅, wherein R^{M1} at each occurrence is independently an optionally substituted C₁₋₄ alkyl or an optionally substituted 3-4 membered ring (including cyclopropyl, cyclobutyl, oxetanyl, azetidinyl, etc.), wherein when substituted, the optionally substituted C₁₋₄ alkyl or 3-4 membered ring is substituted with 1-3 substituents independently selected from halogen (preferably F or Cl), OH, CN, C₁₋₄ alkyl optionally substituted with 1-3 F, C₁₋₄ heteroalkyl optionally substituted with 1-3 F, and 3-4 membered ring (including cyclopropyl, cyclobutyl, oxetanyl, azetidinyl, etc.) optionally substituted with F and/or methyl.

188. The compound of claim 186, or a pharmaceutically acceptable salt thereof, wherein R³⁰ is a 5-membered heteroaryl 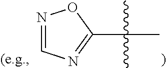 [SMILES: *c1ncno1]
or pyridyl 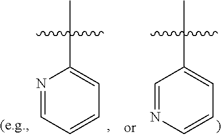 [SMILES: *c1ccccn1 ; *c1cccnc1]
which is substituted with 1-3 (e.g., 1 or 2) R²², as valency permits, wherein R²² at each occurrence is independently F, Cl, CN, R^{M2}, OR^{M2}, SR^{M2}, or SF₅, wherein R^{M2} at each occurrence is independently a C₁₋₄ alkyl optionally substituted with 1-3 F, such as CF₃.

189. The compound of any of claims 168-182, or a pharmaceutically acceptable salt thereof, wherein R³⁰ has a structure according to S-1-A, S-1-B, or S-1-C: 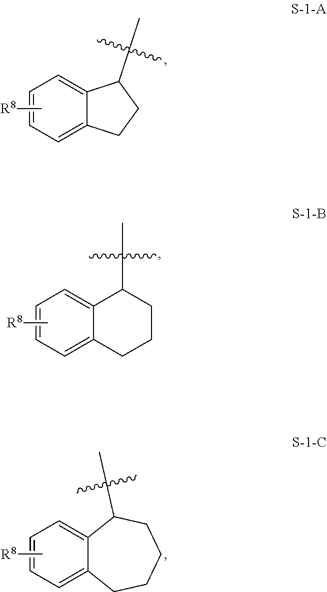
wherein R⁸ is C₁₋₄ alkyl optionally substituted with 1-3 F (such as CH₃, CF₃, etc.), C₁₋₄ alkoxy optionally substituted with 1-3 F (such as OCH₃, OCF₃, etc.), SCF₃, SF₅, cyclopropyl, cyclobutyl, or

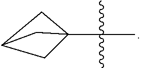 [SMILES: *C12CC(C1)C2]

190. The compound of any of claims 168-182, or a pharmaceutically acceptable salt thereof, wherein the moiety of 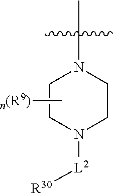
in Formula V is selected from: 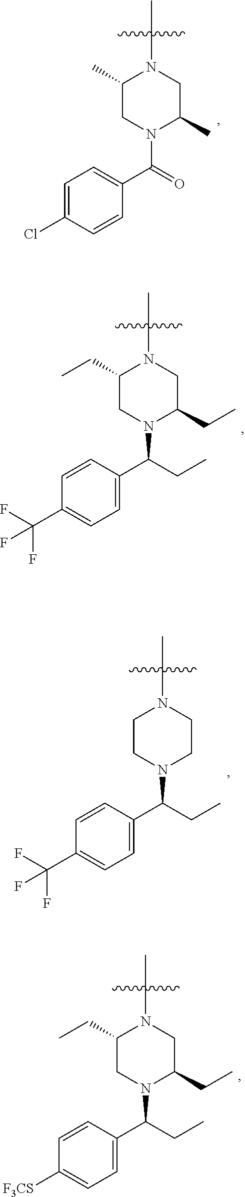 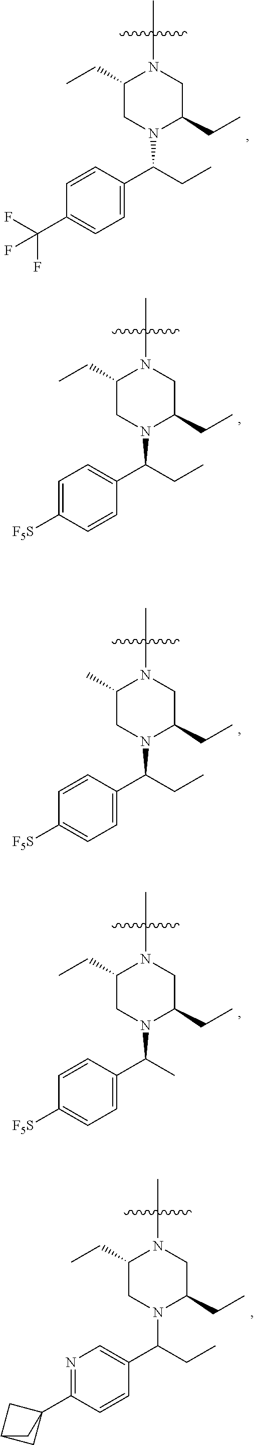 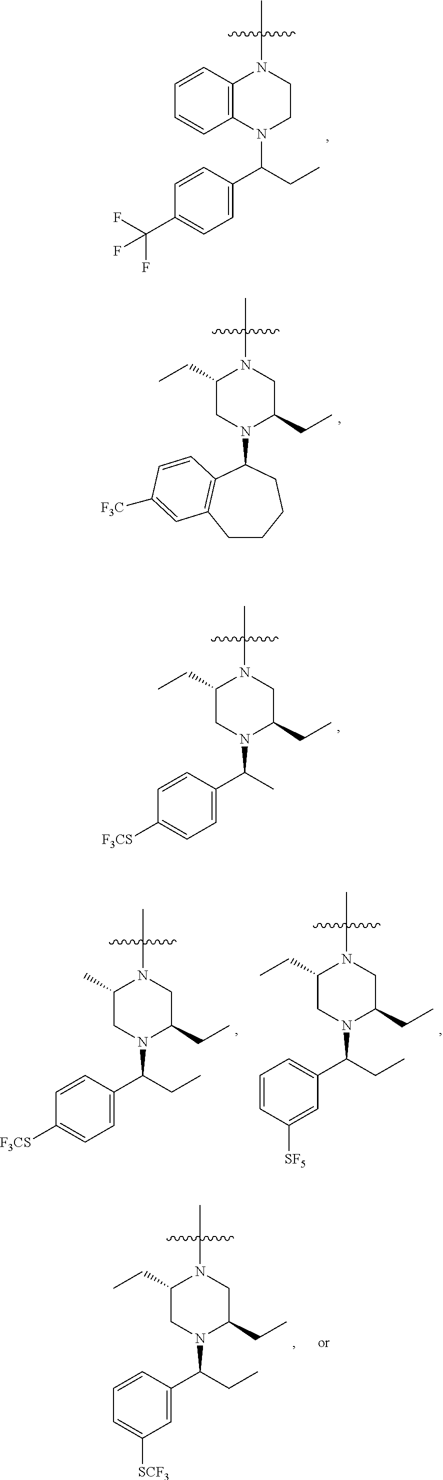 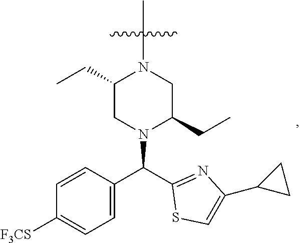
or selected from

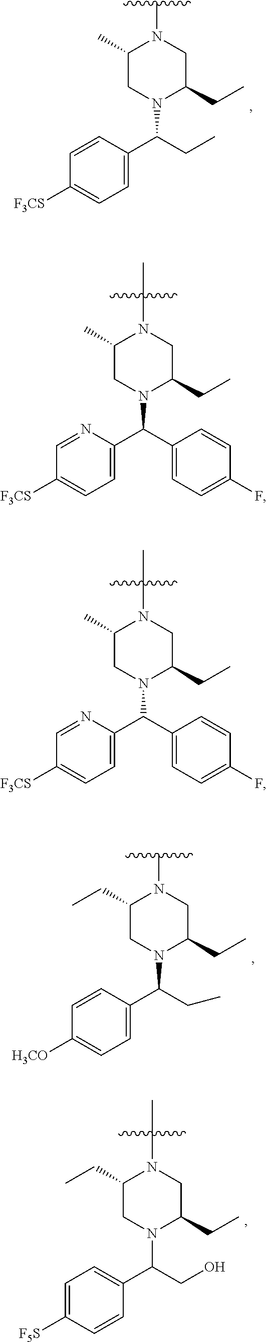

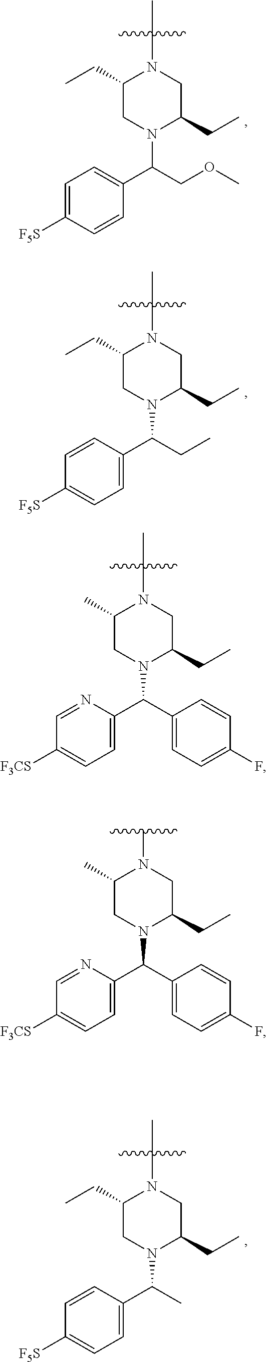

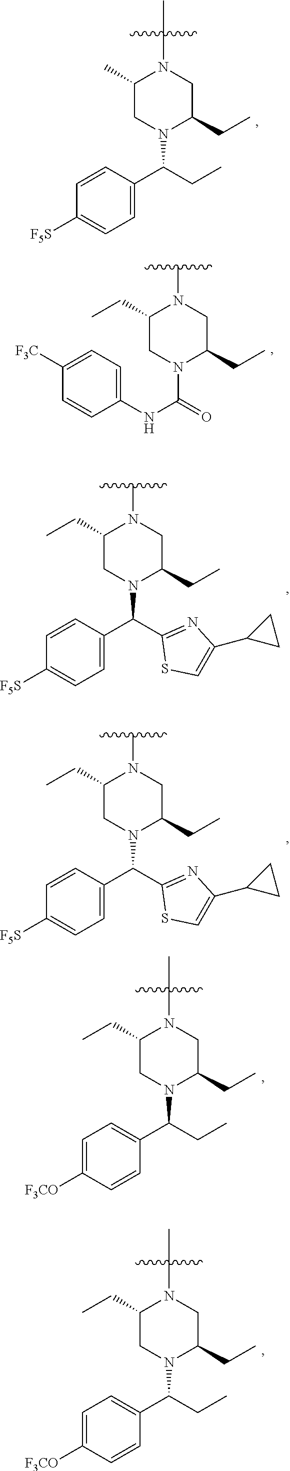

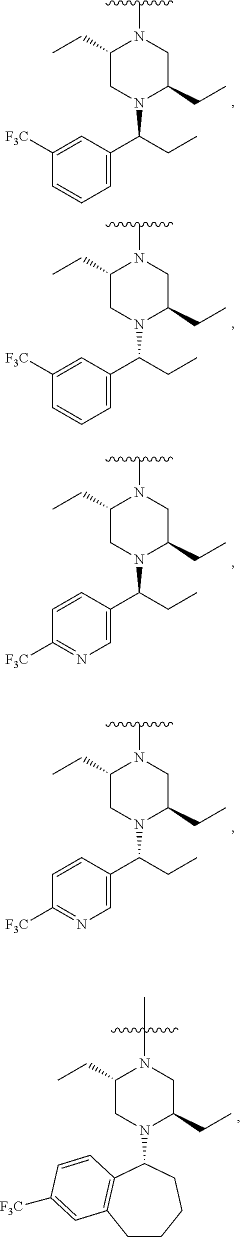 [SMILES: *N1C[C@@H](CC)N([C@@H](CC)c2cccc(C(F)(F)F)c2)C[C@@H]1CC ; *N1C[C@@H](CC)N([C@H](CC)c2cccc(C(F)(F)F)c2)C[C@@H]1CC ; *N1C[C@@H](CC)N([C@@H](CC)c2ccc(C(F)(F)F)nc2)C[C@@H]1CC ; *N1C[C@@H](CC)N([C@H](CC)c2ccc(C(F)(F)F)nc2)C[C@@H]1CC ; *N1C[C@@H](CC)N([C@@H]2CCCCc3cc(C(F)(F)F)ccc32)C[C@@H]1CC]

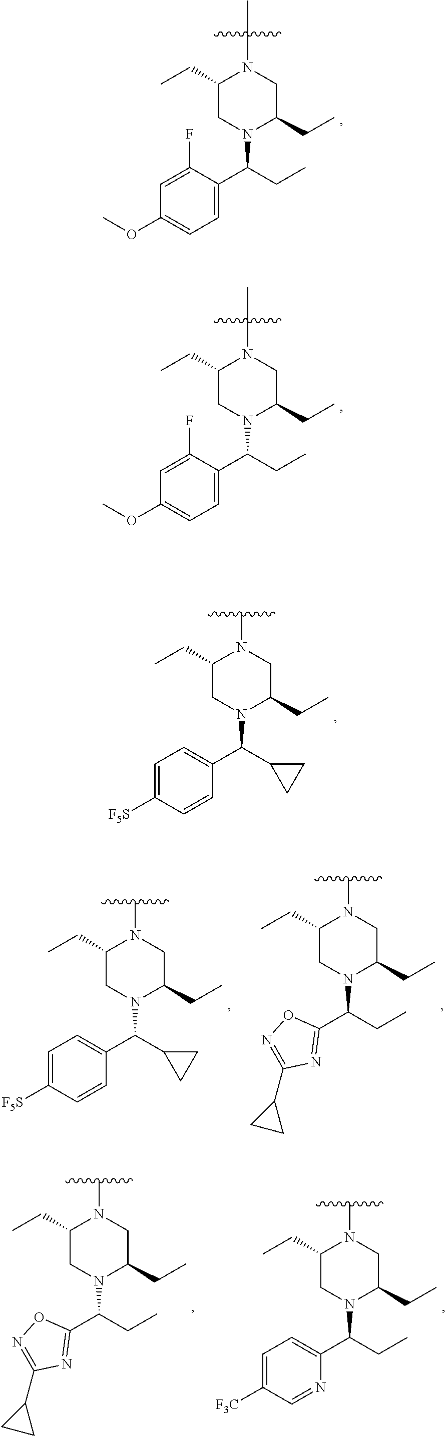

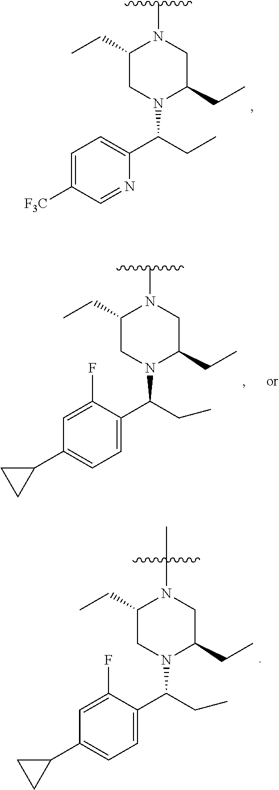 [SMILES: *N1C[C@@H](CC)N([C@H](CC)c2ccc(C(F)(F)F)cn2)C[C@@H]1CC ; *N1C[C@@H](CC)N([C@@H](CC)c2ccc(C3CC3)cc2F)C[C@@H]1CC ; *N1C[C@@H](CC)N([C@H](CC)c2ccc(C3CC3)cc2F)C[C@@H]1CC]

191. A compound selected from Compound No. 1-134 or any of the compounds disclosed in Table A, or a pharmaceutically acceptable salt thereof.

192. A pharmaceutical composition comprising the compound of any of claims 1-191 or a pharmaceutically acceptable salt thereof and optionally a pharmaceutically acceptable excipient.

193. A method for treating a disease comprising the administration to a subject in need thereof a therapeutically-effective amount of at least one compound according to any one of claims 1 to 191, wherein said disease is cancer or a viral infection.

194. The method according to claim 193, wherein said cancer is selected from colon cancer, pancreatic cancer, breast cancer, prostate cancer, lung cancer, ovarian cancer, cervical cancer, renal cancer, cancer of the head and neck, lymphoma, leukemia and melanoma.

195. A method of inhibiting activity of at least one of diacylglycerol kinase selected from diacylglycerol kinase alpha (DGKa) and diacylglycerol kinase zeta (DGKz) comprising administering to a subject in need thereof a therapeutically effective amount of at least one compound according to any one of claims 1 to 191.
